(19) United States
(12) Patent Application Publication
(10) Pub. No.: US 2024/0360178 A1
(43) Pub. Date: Oct. 31, 2024

(54) MACROCYCLIC IMMUNOMODULATORS

(71) Applicant: Bristol-Myers Squibb Company, Princeton, NJ (US)

(72) Inventors: Martin Patrick ALLEN, Flemington, NJ (US); Claudio MAPELLI, Linden, NJ (US); Michael A. POSS, Lawrenceville, NJ (US); Tammy C. WANG, Princeton, NJ (US); Jennifer X. QIAO, Princeton, NJ (US); Yunhi ZHANG, Princeton, NJ (US)

(21) Appl. No.: 18/578,847

(22) PCT Filed: Jul. 12, 2022
(86) PCT No.: PCT/US2022/073630

§ 371 Date: Jan. 12, 2024

Related U.S. Application Data
(60) Provisional application No. 63/220,645, filed on Jul. 12, 2021.

(51) Int. Cl. C07K 7/52; A61K 38/00

(52) U.S. Cl. CPC C07K 7/52; A61K 38/00

(57) ABSTRACT

In accordance with the present disclosure, macrocyclic compounds have been discovered that bind to PD-1 and are capable of inhibiting the interaction of PD-1 with PD-L1. These macrocyclic compounds exhibit in vitro immunomodulatory efficacy thus making them therapeutic candidates for the treatment of various diseases including cancer and infectious diseases.

### CROSS-REFERENCE TO RELATED APPLICATIONS

This application claims priority to U.S. Provisional Application No. 63/220,645, filed Jul. 12, 2021, which is hereby incorporated by reference in its entirety.

### REFERENCE TO SEQUENCE LISTING SUBMITTED ELECTRONICALLY

The content of the electronically submitted sequence listing (Name: 3338_218PC01_Seqlisting; Size: 2,566 bytes; and Date of Creation: Jul. 8, 2022) is herein incorporated by reference in its entirety.

### FIELD

The present disclosure provides macrocyclic compounds that bind to PD-1 and are capable of inhibiting the interaction of PD-1 with PD-L1. These macrocyclic compounds exhibit in vitro immunomodulatory efficacy thus making them therapeutic candidates for the treatment of various diseases including cancer.

### BACKGROUND

Human cancers harbor numerous genetic and epigenetic alterations, generating neoantigens potentially recognizable by the immune system (Sjoblom et al., 2006). The adaptive immune system, comprised of T and B lymphocytes, has powerful anti-cancer potential, with a broad capacity and exquisite specificity to respond to diverse tumor antigens. Further, the immune system demonstrates considerable plasticity and a memory component. The successful harnessing of all these attributes of the adaptive immune system would make immunotherapy unique among all cancer treatment modalities.

The protein Programmed Death 1 (PD-1) is an inhibitory member of the CD28 family of receptors, that also includes CD28, CTLA-4, ICOS and BTLA. PD-1 is expressed on activated B cells, T cells, and myeloid cells (Agata et al., supra; Okazaki et al., Curr. Opin. Immunol., 14:779-782 (2002); Bennett et al., J. Immunol., 170:711-718 (2003)).

The PD-1 protein is a 55 kDa type I transmembrane protein that is part of the Ig gene superfamily (Agata et al., Int. Immunol., 8:765-772 (1996)). PD-1 contains a membrane proximal immunoreceptor tyrosine inhibitory motif (ITIM) and a membrane distal tyrosine-based switch motif (ITSM) (Thomas, M. L., J. Exp. Med., 181:1953-1956 (1995); Vivier, E. et al., Immunol. Today, 18:286-291 (1997)). Although structurally similar to CTLA-4, PD-1 lacks the MYPPY motif that is critical for CD80 CD86 (B7-2) binding. Two ligands for PD-1 have been identified, PD-L1 (B7-H1) and PD-L2 (b7-DC). The activation of T cells expressing PD-1 has been shown to be downregulated upon interaction with cells expressing PD-L1 or PD-L2 (Freeman et al., J. Exp. Med., 192:1027-1034 (2000); Latchman et al., Nat. Immunol., 2:261-268 (2001); Carter et al., Eur. J. Immunol., 32:634-643 (2002)). Both PD-L1 and PD-L2 are B7 protein family members that bind to PD-1, but do not bind to other CD28 family members. The PD-L1 ligand is abundant in a variety of human cancers (Dong et al., Nat. Med., 8:787-789 (2002)). The interaction between PD-1 and PD-L1 results in a decrease in tumor infiltrating lymphocytes, a decrease in T-cell receptor mediated proliferation, and immune evasion by the cancerous cells (Dong et al., J. Mol. Med, 81:281-287 (2003); Blank et al., Cancer Immunol. Immunother., 54:307-314 (2005); Konishi et al., Clin. Cancer Res., 10:5094-5100 (2004)). Immune suppression can be reversed by inhibiting the local interaction of PD-1 with PD-L1, and the effect is additive when the interaction of PD-1 with PD-L2 is blocked as well (Iwai et al., Proc. Natl. Acad. Sci. USA, 99:12293-12297 (2002); Brown et al., J. Immunol., 170:1257-1266 (2003)).

When PD-1 expressing T cells contact cells expressing its ligands, functional activities in response to antigenic stimuli, including proliferation, cytokine secretion, and cytotoxicity, are reduced. PD-1/PD-L1 or PD-L2 interactions down regulate immune responses during resolution of an infection or tumor, or during the development of self tolerance (Keir, M. E. et al., Annu. Rev. Immunol., 26:Epub (2008)). Chronic antigen stimulation, such as that which occurs during tumor disease or chronic infections, results in T cells that express elevated levels of PD-1 and are dysfunctional with respect to activity towards the chronic antigen (reviewed in Kim et al., Curr. Opin. Imm. (2010)). This is termed “T cell exhaustion”. B cells also display PD-1/PD-ligand suppression and “exhaustion”.

In addition to enhancing immunologic responses to chronic antigens, blockade of the PD-1/PD-L1 pathway has also been shown to enhance responses to vaccination, including therapeutic vaccination in the context of chronic infection (Ha, S. J. et al., “Enhancing therapeutic vaccination by blocking PD-1-mediated inhibitory signals during chronic infection”, J. Exp. Med., 205(3):543-555 (2008); Finnefrock, A. C. et al., “PD-1 blockade in rhesus macaques: impact on chronic infection and prophylactic vaccination”, J. Immunol., 182(2):980-987 (2009); Song, M.-Y. et al., “Enhancement of vaccine-induced primary and memory CD8+ t-cell responses by soluble PD-1”, J. Immunother., 34(3):297-306 (2011).

The PD-1 pathway is a key inhibitory molecule in T cell exhaustion that arises from chronic antigen stimulation during tumor disease. Accordingly, agents that block the interaction of PD-1 with PD-L1 are desired.

### SUMMARY

The present disclosure provides macrocyclic compounds which inhibit the PD-1/PD-L1 protein/protein interaction, and are thus useful for the amelioration of various diseases, including cancer.

In one aspect, the present disclosure provides a compound of formula (I)

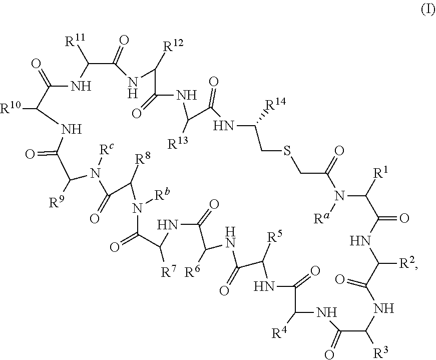

or a pharmaceutically acceptable salt thereof, wherein:
R¹ is selected from hydrogen, C₁-C₆alkyl, amidoC₁-C₆alkyl, aminoC₁-C₆alkyl, arylC₁-C₆alkyl, carboxyC₁-C₆alkyl, (C₃-C₈cycloalkyl)C₁-C₆alkyl, heteroarylC₁-C₆alkyl, hydroxyC₁-C₆alkyl, and NH₂C(X)NHC₁-C₆alkyl, wherein X is O or NH, and wherein the aryl part of the arylC₁-C₆alkyl and the heteroaryl part of the heteroarylC₁-C₆alkyl are optionally substituted with one, two, three, four, or five groups independently selected from C₁-C₆alkoxy, C₁-C₆alkyl, amido, amidoC₁-C₆alkyl, amino, aminoC₁-C₆alkyl, carboxy, carboxyC₁-C₆alkoxy, cyano, halo haloC₁-C₆alkyl, hydroxy, and nitro;
R² is selected from aminoC₁-C₆alkyl, arylC₁-C₆alkyl, and heteroarylC₁-C₆alkyl, wherein the aryl part of the arylC₁-C₆alkyl and the heteroaryl part of the heteroarylC₁-C₆alkyl are optionally substituted with one, two, three, four, or five groups independently selected from C₁-C₆alkoxy, C₁-C₆alkyl, amido, amidoC₁-C₆alkyl, amino, aminoC₁-C₆alkyl, carboxy, carboxyC₁-C₆alkoxy, cyano, halo, haloC₁-C₆alkyl, hydroxy, and nitro;
R³ is selected from carboxyC₁-C₃alkyl, cyanoC₁-C₃alkyl and tetrazolylC₁-C₃alkyl;
R⁴ is selected from arylC₁-C₆alkyl and heteroarylC₁-C₆alkyl, wherein the aryl part of the arylC₁-C₆alkyl and the heteroaryl part of the heteroarylC₁-C₆alkyl are optionally substituted with one, two, three, four, or five groups independently selected from C₁-C₆alkoxy, C₁-C₆alkyl,aminoC₁-C₆alkyl, cyano, halo, haloC₁-C₆alkyl, hydroxy, and nitro;
R⁵ is selected from C₁-C₆alkyl, aryl, arylC₁-C₆alkyl, (C₃-C₈cycloalkyl)C₁-C₆alkyl, and hydroxyC₁-C₆alkyl, wherein the aryl part of the arylC₁-C₆alkyl and the heteroaryl part of the heteroarylC₁-C₆alkyl are optionally substituted with one, two, three, four, or five groups independently selected from C₁-C₆alkoxy, C₁-C₆alkyl, amido, amidoC₁-C₆alkyl, amino, aminoC₁-C₆alkyl, carboxy, carboxyC₁-C₆alkoxy, cyano, halo haloC₁-C₆alkyl, hydroxy, and nitro;
R⁶ is selected from aryl-arylC₁-C₃alkyl, heteroaryl-arylC₁-C₃alkyl, aryl-heteroarylC₁-C₃alkyl, and heteroaryl-heteroarylC₁-C₃alkyl wherein the aryl part of the aryl-arylC₁-C₃alkyl and the aryl-heteroarylC₁-C₃alkyl and the heteroaryl part of the heteroaryl-heteroarylC₁-C₃alkyl and the heteroaryl-arylC₁-C₃alkyl is optionally substituted with one, two, three, four, or five groups independently selected from C₁-C₃alkoxy, C₁-C₃alkyl, amido, amidoC₁-C₃alkyl, amino, aminoC₁-C₃alkyl, carboxy, carboxyC₁-C₃alkoxy, cyano, halo, haloC₁-C₃alkyl, hydroxy, and nitro;
R⁷ is selected from C₁-C₆alkyl, aminoC₁-C₆alkyl, arylC₁-C₆alkyl, carboxyC₁-C₆alkyl, and NH₂C(X)NHC₁-C₆alkyl, wherein X is O or NH, wherein the aryl part of the arylC₁-C₆alkyl is optionally substituted with one, two, three, four, or five groups independently selected from C₁-C₆alkoxy, C₁-C₆alkyl, amido, amidoC₁-C₆alkyl, amino, aminoC₁-C₆alkyl, carboxy, carboxyC₁-C₆alkoxy, cyano, halo, haloC₁-C₆alkyl, hydroxy, hydroxyC₁-C₆alkyl, and nitro;
R³ is selected from C₁-C₆alkyl, aminoC₁-C₆alkyl, carboxyC₁-C₆alkyl, and NH₂C(X)NHC₁-C₆alkyl, wherein X is O or NH; or, R^{b} and R⁸, together with the atoms to which they are attached, form an azetidine, pyrrolidine, piperidine, or morpholine ring, wherein each ring is optionally substituted with an amino or a hydroxy group;
R⁹ is selected from hydrogen, C₁-C₆alkyl, amidoC₁-C₆alkyl, arylC₁-C₆alkyl, hydroxyC₁-C₆alkyl, C₁-C₆alkoxyC₁-C₆alkyl, and NH₂C(X)NHC₁-C₆alkyl, wherein X is O or NH, wherein the aryl part of the arylC₁-C₆alkyl is optionally substituted with one, two, three, four, or five groups independently selected from C₁-C₆alkoxy, C₁-C₆alkyl, amido, amidoC₁-C₆alkyl, amino, aminoC₁-C₆alkyl, carboxy, carboxyC₁-C₆alkoxy, cyano, halo, haloC₁-C₆alkyl, hydroxy, and nitro; or, R^{c} and R⁹, together with the atoms to which they are attached, form an azetidine, pyrrolidine, piperidine, or morpholine ring, wherein each ring is optionally substituted with an amino or a hydroxy group, and wherein each ring is optionally fused to an aryl or heteroaryl ring, wherein the aryl and heteroaryl rings are optionally substituted with one, two, three, or four groups independently selected from C₁-C₆alkoxy, C₁-C₆alkyl, amido, amidoC₁-C₆alkyl, amino, aminoC₁-C₆alkyl, carboxy, carboxyC₁-C₆alkoxy, cyano, halo haloC₁-C₆alkyl, hydroxy, and nitro;
R¹⁰ is selected from C₁-C₆alkyl, amidoC₁-C₆alkyl, aminoC₁-C₆alkyl, carboxyC₁-C₆alkyl, hydroxyC₁-C₆alkyl, and NH₂C(X)NHC₁-C₆alkyl, wherein X is O or NH;
R¹¹ is selected from C₁-C₆alkyl and (C₃-C₈cycloalkyl)C₁-C₆alkyl, wherein the C₁-C₆alkyl and the (C₃-C₈cycloalkyl)C₁-C₆alkyl are optionally substituted with one, two, or three groups independently selected from C₁-C₆alkoxy, cyano, halo, and haloC₁-C₃alkyl;
R¹² is selected from C₁-C₆alkyl, aminoC₁-C₆alkyl, carboxyC₁-C₆alkyl, hydroxyC₁-C₆alkyl, and NH₂C(X)NHC₁-C₆alkyl, wherein X is O or NH;
R¹³ is selected from amidoC₁-C₆alkyl, aminoC₁-C₆alkyl, carboxyC₁-C₆alkyl, heteroarylC₁-C₆alkyl, hydroxyC₁-C₆alkyl, C₁-C₆alkoxyC₁-C₆alkyl, and NH₂C(X)NHC₁-C₆alkyl, wherein X is O or NH, wherein the heteroaryl part of the heteroarylC₁-C₆alkyl is optionally substituted with one, two, three, four, or five groups independently selected from C₁-C₆alkoxy, C₁-C₆alkyl, amido, amidoC₁-C₆alkyl, amino, aminoC₁-C₆alkyl, carboxy, carboxyC₁-C₆alkoxy, cyano, halo, haloC₁-C₆alkyl, hydroxy, and nitro;
R¹⁴ is —C(O)NH₂ or —C(O)NHCR¹⁵R¹⁶C(O)NH₂, wherein:
R¹⁵ is selected from hydrogen and C₁-C₆alkyl; and
R¹⁶ is selected from hydrogen, C₁-C₆alkyl, aminoC₁-C₆alkyl, carboxyC₁-C₆alkyl, (C₃-C₈cycloalkyl)C₁-C₆alkyl, hydroxyC₁-C₆alkyl, and NH₂C(X)NHC₁-C₆alkyl, wherein X is O or NH; or,
R¹⁵ and R¹⁶, together with the carbon atom to which they are attached, form a C₃-C₆cycloalkyl;
R^{a} is hydrogen or C₁-C₆alkyl;
R^{b} is hydrogen, C₁-C₆alkyl, or, R^{b} and R⁸, together with the atoms to which they are attached, form an azetidine, pyrrolidine, piperidine, or morpholine ring, wherein each ring is optionally substituted with an amino or a hydroxy group; and
R^{c} is C₁-C₆alkyl, or R^{c} and R⁹, together with the atoms to which they are attached, form an azetidine, pyrrolidine, piperidine, or morpholine ring, wherein each ring is optionally substituted with an amino or a hydroxy group, and wherein each ring is optionally fused with aryl or heteroaryl ring, wherein the aryl and heteroaryl are optionally substituted with one, two, three, or four groups independently selected from C₁-C₆alkoxy, C₁-C₆alkyl, amido, amidoC₁-C₆alkyl, amino, aminoC₁-C₆alkyl, carboxy, carboxyC₁-C₆alkoxy, cyano, halo haloC₁-C₆alkyl, hydroxy, and nitro.

In some aspects, the present disclosure provides a compound of formula (I), or a pharmaceutically acceptable salt thereof, wherein R² is selected from arylC₁alkyl and heteroarylC₁alkyl, wherein the aryl part of the arylC₁alkyl and the heteroaryl part of the heteroarylC₁alkyl are optionally substituted with one, two, or three groups independently selected from C₁alkoxy, C₁alkyl, amido, amidoC₁alkyl, amino, aminoC₁alkyl, carboxy, carboxyC₁alkoxy, cyano, halo, hydroxy, and nitro.

In some aspects, the present disclosure provides a compound of formula (I), or a pharmaceutically acceptable salt thereof, wherein R⁴ is each selected from arylC₁alkyl and heteroarylC₁alkyl, wherein the aryl part of the arylC₁alkyl and the heteroaryl part of the heteroarylC₁alkyl are optionally substituted with one, two, or three groups independently selected from C₁alkoxy, C₁alkyl, cyano, halo, haloC₁alkyl, and nitro.

In some aspects, the present disclosure provides a compound of formula (I), or a pharmaceutically acceptable salt thereof, wherein R³ is carboxyC₁-C₆alkyl.

In some aspects, the present disclosure provides a compound of formula (I), or a pharmaceutically acceptable salt thereof, wherein R⁵ is selected from C₁-C₆alkyl and arylC₁alkyl, wherein the aryl part of the arylC₁alkyl is optionally substituted with one or two groups independently selected from carboxy and carboxyC₁alkoxy.

In some aspects, the present disclosure provides a compound of formula (I), or a pharmaceutically acceptable salt thereof, wherein R⁶ is unsubstituted aryl-arylC₁alkyl.

In some aspects, the present disclosure provides a compound of formula (I), or a pharmaceutically acceptable salt thereof, wherein R is methyl, or, R^{c} and R⁹, together with the atoms to which they are attached, form an azetidine, morphiline, piperidine, or pyrrolidine ring wherein each ring is optionally substituted with a hydroxy group.

In some aspects, the present disclosure provides a compound of formula (I), or a pharmaceutically acceptable salt thereof, wherein R¹¹ is selected from C₁-C₆alkyl and (C₃-C₈cycloalkyl)C₁alkyl.

In some aspects, the present disclosure provides a compound of formula (I), or a pharmaceutically acceptable salt thereof, wherein R¹² is selected from C₁-C₆alkyl and hydroxyC₁-C₆alkyl.

In some aspects, the present disclosure provides a compound of formula (I), or a pharmaceutically acceptable salt thereof, wherein R¹³ is selected from hydroxyC₁-C₆alkyl and aminoC₁-C₆alkyl.

In some aspects, the present disclosure provides a compound of formula (I), a pharmaceutically acceptable salt thereof, wherein:
R¹ is selected from amidoC₁-C₆alkyl, aminoC₁-C₆alkyl, arylC₁-C₆alkyl, and heteroarylC₁-C₆alkyl, wherein the aryl part of the arylC₁-C₆alkyl and the heteroaryl part of the heteroarylC₁-C₆alkyl are optionally substituted with one, two, three, four, or five groups independently selected from C₁-C₆alkoxy, C₁-C₆alkyl, amido, amidoC₁-C₆alkyl, amino, aminoC₁-C₆alkyl, carboxy, carboxyC₁-C₆alkoxy, cyano, halo haloC₁-C₆alkyl, hydroxy, and nitro;
R² is selected from arylC₁-C₆alkyl and heteroarylC₁-C₆alkyl, wherein the aryl part of the arylC₁-C₆alkyl and the heteroaryl part of the heteroarylC₁-C₆alkyl are optionally substituted with one, two, three, four, or five groups independently selected from C₁-C₆alkoxy, C₁-C₆alkyl, amido, amidoC₁-C₆alkyl, amino, aminoC₁-C₆alkyl, carboxy, carboxyC₁-C₆alkoxy, cyano, halo haloC₁-C₆alkyl, hydroxy, and nitro;
R³ is carboxyC₁alkyl;
R⁴ is selected from arylC₁alkyl and heteroarylC₁alkyl, wherein the aryl part of the arylC₁-C₃alkyl and the heteroaryl part of the heteroarylC₁-C₃alkyl are optionally substituted with one, two, three, four, or five groups independently selected from C₁-C₆alkoxy, C₁-C₆alkyl, aminoC₁-C₆alkyl, cyano, halo haloC₁-C₆alkyl, hydroxy, and nitro;
R⁵ is selected from C₁-C₆alkyl, and arylC₁-C₆alkyl, wherein the aryl part of the arylC₁-C₆alkyl and the heteroaryl part of the heteroarylC₁-C₆alkyl are optionally substituted with one, two, three, four, or five groups independently selected from C₁-C₆alkoxy, C₁-C₆alkyl, amido, amidoC₁-C₆alkyl, amino, aminoC₁-C₆alkyl, carboxy, carboxyC₁-C₆alkoxy, cyano, halo haloC₁-C₆alkyl, hydroxy, and nitro;
R⁶ is aryl-arylC₁-C₃alkyl, wherein the aryl or the heteroaryl part is optionally substituted with one, two, three, four, or five groups independently selected from C₁-C₆alkoxy, C₁-C₆alkyl, amino, aminoC₁-C₆alkyl, cyano, halo haloC₁-C₆alkyl, hydroxy, and nitro;
R⁷ is selected from C₁-C₆alkyl, carboxyC₁-C₆alkyl, NH₂C(O)NHC₁-C₆alkyl;
R⁹ is arylC₁-C₆alkyl
R¹⁰ is selected from amidoC₁-C₆alkyl and aminoC₁-C₆alkyl;
R¹¹ is selected from C₁-C₆alkyl and (C₃-C₈cycloalkyl)C₁-C₆alkyl;
R¹² is selected from C₁-C₆alkyl and hydroxyC₁-C₆alkyl;
R³ is hydroxyC₁-C₆alkyl and aminoC₁-C₆alkyl;
R¹⁴ is —C(O)NHCR¹⁵R¹⁶C(O)NH₂, wherein:
   R¹⁵ is hydrogen; and
   R¹⁶ is selected from C₁-C₆alkyl and aminoC₁-C₆alkyl;
R is hydrogen;
R^{b} is hydrogen or methyl;
R^{c} is C₁-C₆alkyl, or R^{c} and R⁹, together with the atoms to which they are attached, form a pyrrolidine ring of formula:

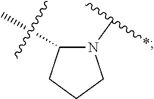

wherein “ [symbol] ” is the point of attachment to the —C(O)NH group, and “ [symbol] ” is the point of attachment to the —C(O)CHR⁸ group.

In some aspects, R¹ is selected from amidoC₁alkyl, aminoC₁₋₂alkyl, arylC₁alkyl, and heteroarylC₁alkyl, wherein the aryl part of the arylC₁alkyl is optionally substituted with a carboxyC₁alkoxy group.

In some aspects, R² is selected from arylC₁alkyl and heteroarylC₁alkyl, wherein the aryl part of the arylC₁alkyl is optionally substituted with one group selected from carboxy, carboxyC₁alkoxy, and cyano.

In some aspects, the aryl is a phenyl or naphthyl group, and the heteroaryl is a benzothienyl, imidazolyl, indolyl, pyrazolyl, pyridinyl, or thiazolyl group.

In some aspects, R³ is carboxyC₁alkyl.

In some aspects, R⁴ is selected from heteroarylC₁alkyl, wherein the heteroaryl is indolyl, and arylC₁alkyl, wherein the aryl part of the arylC₁alkyl is optionally substituted with one group selected from C₁alkoxy and C₁alkyl.

In some aspects, R⁵ is selected from C₃-C₄alkyl, and arylC₁alkyl, wherein the aryl part of the arylC₁alkyl is optionally substituted with one carboxyC₁alkoxy group.

In some aspects, R⁶ is unsubstituted aryl-arylC₁alkyl.

In some aspects, R⁷ is selected from C₃alkyl, carboxyC₂alkyl, and NH₂C(O)NHC₁alkyl.

In some aspects, R⁸ is selected from C₁alkyl and R^{b} is methyl, and R⁸ is selected from aminoC₃alkyl and R^{b} is hydrogen.

In some aspects, R⁹ is arylC₁alkyl and R is methyl, or R^{c} and R⁹, together with the atoms to which they are attached, form a pyrrolidine ring of formula:

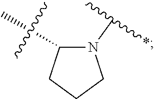

wherein “ [symbol] ” is the point of attachment to the —C(O)NH group, and “ [symbol] ” is the [symbol] point of attachment to the —C(O)CHR⁸ group.

In some aspects, R¹⁰ is selected from amidoC₁alkyl and aminoC₂alkyl.

In some aspects, R¹¹ is selected from C₄alkyl and (C₆cycloalkyl)C₁alkyl.

In some aspects, R¹² is selected from C₃alkyl and hydroxyC₃alkyl.

In some aspects, R¹³ is hydroxyC₁-C₂alkyl.

In some aspects, R¹⁶ is selected from C₁alkyl and aminoC₂alkyl.

In some aspects, the present disclosure provides a pharmaceutical composition comprising a compound of formula (I), or a pharmaceutically acceptable salt thereof.

In some aspects, the present disclosure provides a method of enhancing, stimulating, and/or increasing an immune response in a subject in need thereof, wherein the method comprises administering to the subject a therapeutically effective amount of a compound of formula (I), or a pharmaceutically acceptable salt thereof.

In some aspects, the present disclosure provides a method of blocking the interaction of PD-1 with PD-L1 in a subject, wherein the method comprises administering to the subject a therapeutically effective amount of a compound of formula (I), or a pharmaceutically acceptable salt thereof.

### DETAILED DESCRIPTION

Unless otherwise indicated, any atom with unsatisfied valences is assumed to have hydrogen atoms sufficient to satisfy the valences.

The singular forms “a,” “an,” and “the” include plural referents unless the context dictates otherwise.

As used herein, the term “or” is a logical disjunction (i.e., and/or) and does not indicate an exclusive disjunction unless expressly indicated such as with the terms “either,” “unless,” “alternatively,” and words of similar effect.

As used herein, the phrase “or a pharmaceutically acceptable salt thereof” refers to at least one compound, or at least one salt of the compound, or a combination thereof. For example, “a compound of Formula (I) or a pharmaceutically acceptable salt thereof” includes, but is not limited to, a compound of Formula (I), two compounds of Formula (I), a pharmaceutically acceptable salt of a compound of Formula (I), a compound of Formula (I) and one or more pharmaceutically acceptable salts of the compound of Formula (I), and two or more pharmaceutically acceptable salts of a compound of Formula (I).

The term “C₁-C₆alkoxy”, as used herein, refers to a C₁-C₆alkyl group attached to the parent molecular moiety through an oxygen atom.

The term “C₁-C₆alkoxyC₁-C₆alkyl”, as used herein, refers to a C₁-C₆alkoxy group attached to the parent molecular moiety through a C₁-C₆alkyl group.

The term “C₁-C₃alkyl”, as used herein, refers to a group derived from a straight or branched chain saturated hydrocarbon containing from one to three carbon atoms.

The term “C₁-C₆alkyl”, as used herein, refers to a group derived from a straight or branched chain saturated hydrocarbon containing from one to six carbon atoms.

The term “amido,” as used herein, refers to —C(O)NH₂.

The term “amidoC₁-C₆alkyl,” as used herein, refers to an amido group attached to the parent molecular moiety through a C₁-C₆alkyl group.

The term “amino,” as used herein, refers to —NH₂.

The term “aminoC₁-C₆alkyl,” as used herein, refers to an amino group attached to the parent molecular moiety through a C₁-C₆alkyl group.

The term “aryl,” as used herein, refers to a phenyl group, or a bicyclic fused ring system wherein one or both of the rings is a phenyl group. Bicyclic fused ring systems consist of a phenyl group fused to a four- to six-membered aromatic or non-aromatic carbocyclic ring. The aryl groups of the present disclosure can be attached to the parent molecular moiety through any substitutable carbon atom in the group. Representative examples of aryl groups include, but are not limited to, indanyl, indenyl, naphthyl, phenyl, and tetrahydronaphthyl.

The term “arylC₁-C₆alkyl,” as used herein, refers to an aryl group attached to the parent molecular moiety through a C₁-C₆alkyl group.

The term “aryl-aryl,” as used herein, refers to an aryl group attached to the parent molecular moiety through a second aryl group.

The term “aryl-arylC₁-C₃alkyl,” as used herein, refers to an aryl-aryl group attached to the parent molecular moiety through a C₁-C₃alkyl group.

The term “aryl-heteroaryl,” as used herein, refers to an aryl group attached to the parent molecular moiety through a heteroaryl group.

The term “aryl-heteroarylC₁-C₃alkyl,” as used herein, refers to an aryl-heteroaryl group attached to the parent molecular moiety through a C₁-C₃alkyl group.

The term “carboxy”, as used herein, refers to —CO₂H.

The term “carboxyC₁-C₆alkoxy,” as used herein, refers to a carboxyC₁-C₆alkyl group attached to the parent molecular moiety through an oxygen atom.

The term “carboxyC₁-C₃alkyl”, as used herein, refers to a carboxy group attached to the parent molecular moiety through a C₁-C₃alkyl group.

The term “carboxyC₁-C₆alkyl”, as used herein, refers to a carboxy group attached to the parent molecular moiety through a C₁-C₆alkyl group.

The term “cyano”, as used herein, refers to —CN.

The term “cyanoC₁-C₃alkyl”, as used herein, refers to a cyano group attached to the parent molecular moiety through a C₁-C₃alkyl group.

The term “C₃-C₈cycloalkyl”, as used herein, refers to a saturated monocyclic or bicyclic hydrocarbon ring system having three to eight carbon atoms and zero heteroatoms. The bicyclic rings can be fused, spirocyclic, or bridged. Representative examples of cycloalkyl groups include, but are not limited to, cyclopropyl, cyclopentyl, octahydropentalene, and bicyclo[3.1.1]heptyl.

The term “(C₃-C₈cycloalkyl)C₁-C₆alkyl”, as used herein, refers to a C₃-C₈cycloalkyl group attached to the parent molecular moiety through a C₁-C₆alkyl group.

The terms “halo” and “halogen”, as used herein, refer to F, Cl, Br, or I.

The term “haloC₁-C₆alkyl”, as used herein, refers to a C₁-C₆alkyl group substituted with one, two, or three halogen atoms.

The term “heteroaryl,” as used herein, refers to an aromatic five- or six-membered ring where at least one atom is selected from N, O, and S, and the remaining atoms are carbon. The term “heteroaryl” also includes bicyclic systems where a heteroaryl ring is fused to a four- to six-membered aromatic or non-aromatic ring containing zero, one, or two additional heteroatoms selected from N, O, and S; and tricyclic systems where a bicyclic system is fused to a four- to six-membered aromatic or non-aromatic ring containing zero, one, or two additional heteroatoms selected from N, O, and S. The heteroaryl groups are attached to the parent molecular moiety through any substitutable carbon or nitrogen atom in the group. Representative examples of heteroaryl groups include, but are not limited to, alloxazine, benzo[1,2-d:4,5-d]bisthiazole, benzoxadiazolyl, benzoxazolyl, benzofuranyl, benzothienyl, furanyl, imidazolyl, indazolyl, indolyl, isoxazolyl, isoquinolinyl, isothiazolyl, naphthyridinyl, oxadiazolyl, oxazolyl, purine, pyridinyl, pyridazinyl, pyrimidinyl, pyrazinyl, pyrazolyl, pyrrolyl, quinolinyl, thiazolyl, thienopyridinyl, thienyl, triazolyl, thiadiazolyl, and triazinyl.

The term “heteroarylC₁-C₆alkyl,” as used herein, refers to a heteroaryl group attached to the parent molecular moiety through a C₁-C₆alkyl group.

The term “heteroaryl-aryl,” as used herein, refers to a heteroaryl group attached to the parent molecular moiety through an aryl group.

The term “heteroaryl-arylC₁-C₃alkyl,” as used herein, refers to a heteroaryl-aryl group attached to the parent molecular moiety through a C₁-C₃alkyl group.

The term “heteroaryl-heteroaryl,” as used herein, refers to a heteroaryl group attached to the parent molecular moiety through a heteroaryl group.

The term “heteroaryl-heteroarylC₁-C₃alkyl,” as used herein, refers to a heteroaryl-heteroaryl group attached to the parent molecular moiety through a C₁-C₃alkyl group.

The term “hydroxy,” as used herein, refers to —OH.

The term “hydroxyC₁-C₆alkyl,” as used herein, refers to a hydroxy group attached to the parent molecular moiety through a C₁-C₆alkyl group.

The term “nitro”, as used herein, refers to —NO₂.

The term “tetrazolylC₁-C₃alkyl,” as used herein, refers to a tetrazolyl group attached to the parent molecular moiety through a C₁-C₃alkyl group.

The term “immune response” refers to the action of, for example, lymphocytes, antigen presenting cells, phagocytic cells, granulocytes, and soluble macromolecules that results in selective damage to, destruction of, or elimination from the human body of invading pathogens, cells or tissues infected with pathogens, cancerous cells, or, in cases of autoimmunity or pathological inflammation, normal human cells or tissues.

The terms “Programmed Death Ligand 1”, “Programmed Cell Death Ligand 1”, “PD-L1”, “PDL1”, “hPD-L1”, “hPD-L1”, and “B7-H1” are used interchangeably, and include variants, isoforms, species homologs of human PD-L1, and analogs having at least one common epitope with PD-L1. The complete PD-L1 sequence can be found under GENBANK® Accession No. NP_054862.

The terms “Programmed Death 1”, “Programmed Cell Death 1”, “Protein PD-1”, “PD-1”, “PD1”, “hPD-1” and “hPD-1” are used interchangeably, and include variants, isoforms, species homologs of human PD-1, and analogs having at least one common epitope with PD-1. The complete PD-1 sequence can be found under GENBANK® Accession No. U64863.

The term “treating” refers to i) inhibiting the disease, disorder, or condition, i.e., arresting its development; and/or ii) relieving the disease, disorder, or condition, i.e., causing regression of the disease, disorder, and/or condition and/or symptoms associated with the disease, disorder, and/or condition.

The present disclosure is intended to include all isotopes of atoms occurring in the present compounds. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the disclosure can generally be prepared by conventional techniques known to those skilled in the art or by processes analogous to those described herein, using an appropriate isotopically-labeled reagent in place of the non-labeled reagent otherwise employed. Such compounds can have a variety of potential uses, for example as standards and reagents in determining biological activity. In the case of stable isotopes, such compounds can have the potential to favorably modify biological, pharmacological, or pharmacokinetic properties.

An additional aspect of the subject matter described herein is the use of the disclosed compounds as radiolabeled ligands for development of ligand binding assays or for monitoring of in vivo adsorption, metabolism, distribution, receptor binding or occupancy, or compound disposition. For example, a macrocyclic compound described herein can be prepared using a radioactive isotope and the resulting radiolabeled compound can be used to develop a binding assay or for metabolism studies. Alternatively, and for the same purpose, a macrocyclic compound described herein can be converted to a radiolabeled form by catalytic tritiation using methods known to those skilled in the art.

The macrocyclic compounds of the present disclosure can also be used as PET imaging agents by adding a radioactive tracer using methods known to those skilled in the art.

Those of ordinary skill in the art are aware that an amino acid includes a compound represented by the general structure:

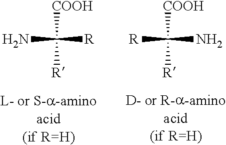

where R and R′ are as discussed herein. Unless otherwise indicated, the term “amino acid” as employed herein, alone or as part of another group, includes, without limitation, an amino group and a carboxyl group linked to the same carbon, referred to as “α” carbon, where R and/or R′ can be a natural or an un-natural side chain, including hydrogen. The absolute “S” configuration at the “α” carbon is commonly referred to as the “L” or “natural” configuration. In the case where both the “R” and the “R′” (prime) substituents equal hydrogen, the amino acid is glycine and is not chiral.

Where not specifically designated, the amino acids described herein can be D- or L-stereochemistry and can be substituted as described elsewhere in the disclosure. It should be understood that when stereochemistry is not specified, the present disclosure encompasses all stereochemical isomeric forms, or mixtures thereof, which possess the ability to inhibit the interaction between PD-1 and PD-L1. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, or direct separation of enantiomers on chiral chromatographic columns. Starting compounds of particular stereochemistry are either commercially available or can be made and resolved by techniques known in the art.

Certain compounds of the present disclosure can exist in different stable conformational forms which may be separable. Torsional asymmetry due to restricted rotation about an asymmetric single bond, for example because of steric hindrance or ring strain, may permit separation of different conformers. The present disclosure includes each conformational isomer of these compounds and mixtures thereof.

Certain compounds of the present disclosure can exist as tautomers, which are compounds produced by the phenomenon where a proton of a molecule shifts to a different atom within that molecule. The term “tautomer” also refers to one of two or more structural isomers that exist in equilibrium and are readily converted from one isomer to another. All tautomers of the compounds described herein are included within the present disclosure.

The pharmaceutical compounds of the disclosure can include one or more pharmaceutically acceptable salts. A “pharmaceutically acceptable salt” refers to a salt that retains the desired biological activity of the parent compound and does not impart any undesired toxicological effects (see e.g., Berge, S. M. et al., J. Pharm. Sci., 66:1-19 (1977)). The salts can be obtained during the final isolation and purification of the compounds described herein, or separately be reacting a free base function of the compound with a suitable acid or by reacting an acidic group of the compound with a suitable base. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

Administration of a therapeutic agent described herein includes, without limitation, administration of a therapeutically effective amount of therapeutic agent. The term “therapeutically effective amount” as used herein refers, without limitation, to an amount of a therapeutic agent to treat a condition treatable by administration of a composition comprising the PD-1/PD-L1 binding inhibitors described herein. That amount is the amount sufficient to exhibit a detectable therapeutic or ameliorative effect. The effect can include, for example and without limitation, treatment of the conditions listed herein. The precise effective amount for a subject will depend upon the subject's size and health, the nature and extent of the condition being treated, recommendations of the treating physician, and therapeutics or combination of therapeutics selected for administration.

For administration of the macrocyclic peptides described herein, the dosage ranges from about 0.0001 to 100 mg/kg, and more usually 0.01 to 40 mg/kg, of the host body weight. For example dosages can be 0.3 mg/kg body weight, 1 mg/kg body weight, 3 mg/kg body weight, 5 mg/kg body weight, 10 mg/kg body weight, 20 mg/kg body weight, 30 mg/kg body weight, 40 mg/kg body weight, or within the range of 10-40 mg/kg.

In another aspect, the disclosure pertains to methods of inhibiting growth of tumor cells in a subject using the macrocyclic compounds of the present disclosure. In certain aspects, the compounds of the present disclosure are capable of binding to PD-1, disrupting the interaction between PD-1 and PD-L1, competing with the binding of PD-1 with certain anti-PD-1 monoclonal antibodies that are known to block the interaction with PD-L1, and enhancing CMV-specific T cell IFNγ secretion. As a result, the compounds of the present disclosure can be useful for modifying an immune response, treating diseases such as cancer, stimulating a protective autoimmune response, or to stimulate antigen-specific immune responses (e.g., by co-administration of PD-L1 blocking compounds with an antigen of interest). For example, the compounds of the present disclosure can be used to treat cancers selected from melanoma, renal cell carcinoma, squamous non-small cell lung cancer (NSCLC), non-squamous NSCLC, colorectal cancer, castration-resistant prostate cancer, ovarian cancer, gastric cancer, hepatocellular carcinoma, pancreatic carcinoma, squamous cell carcinoma of the head and neck, carcinomas of the esophagus, gastrointestinal tract and breast, and hematological malignancies.

Compounds of the present disclosure can also be used in treating infectious diseases, such as those caused by a virus. Examples of such viruses include, but are not limited to, HIV, Hepatitis A, Hepatitis B, Hepatitis C, herpes viruses, and influenza.

Compounds of the present disclosure can also be used in treating septic shock.

### Pharmaceutical Compositions

In another aspect, the present disclosure provides a composition, e.g., a pharmaceutical composition, containing one or a combination of the compounds described within the present disclosure, formulated together with a pharmaceutically acceptable carrier. Pharmaceutical compositions of the disclosure also can be administered in combination therapy, i.e., combined with other agents. For example, the combination therapy can include a macrocyclic compound combined with at least one other anti-inflammatory or immunosuppressant agent. Examples of therapeutic agents that can be used in combination therapy are described in greater detail below in the section on uses of the compounds of the disclosure.

As used herein, “pharmaceutically acceptable carrier” includes any and all solvents, dispersion media, coatings, antibacterial and antifungal agents, isotonic and absorption delaying agents, and the like that are physiologically compatible. In some aspects, the carrier is suitable for intravenous, intramuscular, subcutaneous, parenteral, spinal or epidermal administration (e.g., by injection or infusion). Depending on the route of administration, the active compound can be coated in a material to protect the compound from the action of acids and other natural conditions that can inactivate the compound.

A pharmaceutical composition of the disclosure also can include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The pharmaceutical compositions of the present disclosure can be administered via one or more routes of administration using one or more of a variety of methods known in the art. As will be appreciated by the skilled artisan, the route and/or mode of administration will vary depending upon the desired results. In some aspects, the routes of administration for macrocyclic compounds of the disclosure include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, some methods of preparation are vacuum drying and freeze-drying (lyophilization) that yield a powder of the active ingredient plus any additional desired ingredient from a previously sterile-filtered solution thereof.

Examples of suitable aqueous and non-aqueous carriers that can be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions can also contain adjuvants such as preservatives, wetting agents, emulsifying agents and dispersing agents. Prevention of presence of microorganisms can be ensured both by sterilization procedures, supra, and by the inclusion of various antibacterial and antifungal agents, for example, paraben, chlorobutanol, phenol sorbic acid, and the like. It can also be desirable to include isotonic agents, such as sugars, sodium chloride, and the like into the compositions. In addition, prolonged absorption of the injectable pharmaceutical form can be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the disclosure is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be desirable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Alternatively, the compounds of the disclosure can be administered via a non-parenteral route, such as a topical, epidermal or mucosal route of administration, for example, intranasally, orally, vaginally, rectally, sublingually or topically.

Any pharmaceutical composition contemplated herein can, for example, be delivered orally via any acceptable and suitable oral preparation. Exemplary oral preparations include, but are not limited to, for example, tablets, troches, lozenges, aqueous and oily suspensions, dispersible powders or granules, emulsions, hard and soft capsules, liquid capsules, syrups, and elixirs. Pharmaceutical compositions intended for oral administration can be prepared according to any methods known in the art for manufacturing pharmaceutical compositions intended for oral administration. In order to provide pharmaceutically palatable preparations, a pharmaceutical composition in accordance with the disclosure can contain at least one agent selected from sweetening agents, flavoring agents, coloring agents, demulcents, antioxidants, and preserving agents.

A tablet can, for example, be prepared by admixing at least one compound of Formula (I) and/or at least one pharmaceutically acceptable salt thereof with at least one non-toxic pharmaceutically acceptable excipient suitable for the manufacture of tablets. Exemplary excipients include, but are not limited to, for example, inert diluents, such as, for example, calcium carbonate, sodium carbonate, lactose, calcium phosphate, and sodium phosphate; granulating and disintegrating agents, such as, for example, microcrystalline cellulose, sodium crosscarmellose, corn starch, and alginic acid; binding agents such as, for example, starch, gelatin, polyvinyl-pyrrolidone, and acacia; and lubricating agents, such as, for example, magnesium stearate, stearic acid, and talc. Additionally, a tablet can either be uncoated, or coated by known techniques to either mask the bad taste of an unpleasant tasting drug, or delay disintegration and absorption of the active ingredient in the gastrointestinal tract thereby sustaining the effects of the active ingredient for a longer period. Exemplary water soluble taste masking materials include, but are not limited to, hydroxypropyl-methylcellulose and hydroxypropyl-cellulose. Exemplary time delay materials include, but are not limited to, ethyl cellulose and cellulose acetate butyrate.

Hard gelatin capsules can, for example, be prepared by mixing at least one compound of Formula (I) and/or at least one salt thereof with at least one inert solid diluent, such as, for example, calcium carbonate; calcium phosphate; and kaolin.

Soft gelatin capsules can, for example, be prepared by mixing at least one compound of Formula (I) and/or at least one pharmaceutically acceptable salt thereof with at least one water soluble carrier, such as, for example, polyethylene glycol; and at least one oil medium, such as, for example, peanut oil, liquid paraffin, and olive oil.

An aqueous suspension can be prepared, for example, by admixing at least one compound of Formula (I) and/or at least one pharmaceutically acceptable salt thereof with at least one excipient suitable for the manufacture of an aqueous suspension, including, but are not limited to, for example, suspending agents, such as, for example, sodium carboxymethylcellulose, methylcellulose, hydroxypropylmethyl-cellulose, sodium alginate, alginic acid, polyvinyl-pyrrolidone, gum tragacanth, and gum acacia; dispersing or wetting agents, such as, for example, a naturally-occurring phosphatide, e.g., lecithin; condensation products of alkylene oxide with fatty acids, such as, for example, polyoxyethylene stearate; condensation products of ethylene oxide with long chain aliphatic alcohols, such as, for example, heptadecathylene-oxycetanol; condensation products of ethylene oxide with partial esters derived from fatty acids and hexitol, such as, for example, polyoxyethylene sorbitol monooleate; and condensation products of ethylene oxide with partial esters derived from fatty acids and hexitol anhydrides, such as, for example, polyethylene sorbitan monooleate. An aqueous suspension can also contain at least one preservative, such as, for example, ethyl and n-propyl p-hydroxybenzoate; at least one coloring agent; at least one flavoring agent; and/or at least one sweetening agent, including but not limited to, for example, sucrose, saccharin, and aspartame.

Oily suspensions can, for example, be prepared by suspending at least one compound of Formula (I) and/or at least one pharmaceutically acceptable salt thereof in either a vegetable oil, such as, for example, arachis oil, sesame oil, and coconut oil; or in mineral oil, such as, for example, liquid paraffin. An oily suspension can also contain at least one thickening agent, such as, for example, beeswax, hard paraffin, and cetyl alcohol. In order to provide a palatable oily suspension, at least one of the sweetening agents already described herein above, and/or at least one flavoring agent can be added to the oily suspension. An oily suspension can further contain at least one preservative, including, but not limited to, for example, an anti-oxidant, such as, for example, butylated hydroxyanisol, and alpha-tocopherol.

Dispersible powders and granules can, for example, be prepared by admixing at least one compound of Formula (I) and/or at least one pharmaceutically acceptable salt thereof with at least one dispersing and/or wetting agent, at least one suspending agent, and/or at least one preservative. Suitable dispersing agents, wetting agents, and suspending agents are already described above. Exemplary preservatives include, but are not limited to, for example, anti-oxidants, e.g., ascorbic acid. In addition, dispersible powders and granules can also contain at least one excipient, including, but not limited to, for example, sweetening agents, flavoring agents, and coloring agents.

An emulsion of at least one compound of Formula (I) and/or at least one pharmaceutically acceptable salt thereof can, for example, be prepared as an oil-in-water emulsion. The oily phase of the emulsions comprising the compounds of Formula (I) can be constituted from known ingredients in a known manner. The oil phase can be provided by, but is not limited to, for example, a vegetable oil, such as, for example, olive oil and arachis oil; a mineral oil, such as, for example, liquid paraffin; and mixtures thereof. While the phase can comprise merely an emulsifier, it can comprise a mixture of at least one emulsifier with a fat or an oil or with both a fat and an oil. Suitable emulsifying agents include, but are not limited to, for example, naturally-occurring phosphatides, e.g., soy bean lecithin, esters or partial esters derived from fatty acids and hexitol anhydrides, such as, for example sorbitan monoleate, and condensation products of partial esters with ethylene oxide, such as, for example, polyoxyethylene sorbitan monooleate. In some aspects, a hydrophilic emulsifier is included together with a lipophilic emulsifier which acts as a stabilizer. It is also sometimes desirable to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations. An emulsion can also contain a sweetening agent, a flavoring agent, a preservative, and/or an antioxidant. Emulsifiers and emulsion stabilizers suitable for use in the formulation of the present disclosure include Tween 60, Span 80, cetostearyl alcohol, myristyl alcohol, glyceryl monostearate, sodium lauryl sulfate, glyceral disterate alone or with a wax, or other materials well known in the art.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Robinson, J. R., ed., Sustained and Controlled Release Drug Delivery Systems, Marcel Dekker, Inc., New York (1978).

Therapeutic compositions can be administered with medical devices known in the art. For example, in one aspect, a therapeutic composition of the disclosure can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the present disclosure include: U.S. Pat. No. 4,487,603, which discloses an implantable micro-infusion pump for dispensing medication at a controlled rate; U.S. Pat. No. 4,486,194, which discloses a therapeutic device for administering medication through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In certain aspects, the compounds of the disclosure can be formulated to ensure proper distribution in vivo. For example, the blood-brain barrier (BBB) excludes many highly hydrophilic compounds. To ensure that therapeutic compounds of the disclosure cross the BBB (if desired), they can be formulated, for example, in liposomes. For methods of manufacturing liposomes, see, e.g., U.S. Pat. Nos. 4,522,811, 5,374,548, and 5,399,331. The liposomes can comprise one or more moieties which are selectively transported into specific cells or organs, thus enhance targeted drug delivery (see, e.g., Ranade, V. V., J. Clin. Pharmacol., 29:685 (1989)). Exemplary targeting moieties include folate or biotin (see, e.g., U.S. Pat. No. 5,416,016 to Low et al.); mannosides (Umezawa et al., Biochem. Biophys. Res. Commun., 153:1038 (1988)); macrocyclic compounds (Bloeman, P. G. et al., FEBS Lett., 357:140 (1995); Owais, M. et al., Antimicrob. Agents Chemother., 39:180 (1995)); surfactant protein A receptor (Briscoe et al., Am. J. Physiol., 1233:134 (1995)); p 120 (Schreier et al., J. Biol. Chem., 269:9090 (1994)); see also Keinanen, K. et al., FEBS Lett., 346:123 (1994); Killion, J. J. et al., Immunomethods 4:273 (1994).

In certain aspects, the compounds of the present disclosure can be administered parenterally, i.e., by injection, including, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and/or infusion.

In some aspects, the compounds of the present disclosure can be administered orally, i.e, via a gelatin capsule, tablet, hard or soft capsule, or a liquid capsule. The compounds can be made by methods known in the art including those described below and including variations within the skill of the art. Some reagents and intermediates are known in the art. Other reagents and intermediates can be made by methods known in the art using readily available materials. Any variables (e.g. numbered “R” substituents) used to describe the synthesis of the compounds are intended only to illustrate how to make the compounds and are not to be confused with variables used in the claims or in other sections of the specification. The following methods are for illustrative purposes and are not intended to limit the scope of the disclosure.

Abbreviations used in the schemes generally follow conventions used in the art. Chemical abbreviations used in the specification and examples are defined as follows: FMOC for fluorenylmethoxycarbonyl; HOBt for 1-hydroxybenzotriazole hydrate; HOAT for 1-hydroxy-7-azabenzotriazole; DIC for diisopropylcarbodiimide; HBTU for 2-(1H-benzotriazol-1-yl)-1,1,3,3-tetramethyluronium hexafluorophosphate, hexafluorophosphate benzotriazole tetramethyl uronium; BOP for benzotriazol-1-yloxytris(dimethylamino)phosphonium hexafluorophosphate; PyBOP for benzotriazol-1-yl-oxytripyrrolidinophosphonium hexafluorophosphate; HCTU for 1-[bis(dimethylamino)methylen]-5-chlorobenzotriazolium 3-oxide hexafluorophosphate or N,N,N′,N′-tetramethyl-O-(6-chloro-1H-benzotriazol-1-yl)uronium hexafluorophosphate; HATU for 1-[bis(dimethylamino)methylene]-1H-1,2,3-triazolo[4,5-b]pyridinium 3-oxid hexafluorophosphate or N-[(dimethylamino)-1H-1,2,3-triazolo-[4,5-b]pyridin-1-ylmethylene]-N-methylmethanaminium hexafluorophosphate N-oxide; iPrNEt₂ or DIPEA or DIEAfor diisopropylethylamine; DMF for N,N-dimethylformamide; TIS for triisopropylsilane; DTT for dithiothreitol (Cleland's reagent); TCEP for tris-2(-carboxyethyl)-phosphine; Et₂O for diethyl ether; DMSO for dimethylsulfoxide; CAN for ceric ammonium nitrate; DCM for dichloromethane; DVB for divinylbenzene; Pbf for 2,2,4,6,7-Pentamethyldihydrobenzofuran-5-sulfonyl chloride; Trt for trityl; t-Bu for tert-butyl; BOC for tert-butoxycarbonyl; Me for methyl; NMM for N-methylmorpholine; RT for room temperature or retention time (context will dictate); min or mins for minutes; h or hr or hrs for hours; NOS-CL for 4-nitrobenzenesulfonyl chloride; DBU for 1,8-diazabicyclo[5.4.0]undec-7-ene; THF for tetrahydrfuran; dtbpf for [1,1′-bis(di-tert-butylphosphino)ferrocene]; MeOH for methanol; Fmoc-OSu for N-(9-Fluorenylmethoxycarbonyloxy)succinimide, 9-fluorenylmethyl-succinimidyl carbonate; Ac for acetyl; SPhos for 2-dicyclohexylphosphino-2′,6′-dimethoxybiphenyl; DBA for tris(dibenzylideneacetone); TMS for trimethylsilyl; Hex for hexyl; XPhos for 2-dicyclohexylphosphino-2′,4′,6′-triisopropylbiphenyl; TEMPO for (2,2,6,6-tetramethylpiperidin-1-yl)oxyl or (2,2,6,6-tetramethylpiperidin-1-yl)oxidanyl; ACN or MeCN for acetonitrile; Ethyl Acetate or EtOAc for ethyl acetate; Et₃N or TEA for trimethylamine; PE for petroleum ether; KHMDS for potassium hexamethyldisilazide; HFIP or HFIPA for hexafluoroisopropanol; TCNHPI for N-hydroxytetrachlorophthalimide; DIAD for diisopropyl azodicarboxylate; DtBuPf for 1,1′-bis(di-tert-butylphosphino)ferrocene; and t-Bu for tert-butyl.

### Macrocycle Synthesis

The macrocyclic peptides of the present disclosure can be produced by methods known in the art, such as they can be synthesized chemically, recombinantly in a cell free system, recombinantly within a cell or can be isolated from a biological source. Chemical synthesis of a macrocyclic peptide of the present disclosure can be carried out using a variety of art recognized methods, including stepwise solid phase synthesis, semi-synthesis through the conformationally-assisted re-ligation of peptide fragments, enzymatic ligation of cloned or synthetic peptide segments, and chemical ligation. A preferred method to synthesize the macrocyclic peptides and analogs thereof described herein is chemical synthesis using various solid-phase techniques such as those described in Chan, W. C. et al, eds., Fmoc Solid Phase Synthesis, Oxford University Press, Oxford (2000); Barany, G. et al, The Peptides: Analysis, Synthesis, Biology, Vol. 2: “Special Methods in Peptide Synthesis, Part A”, pp. 3-284, Gross, E. et al, eds., Academic Press, New York (1980); in Atherton, E., Sheppard, R. C. Solid Phase Peptide Synthesis: A Practical Approach, IRL Press, Oxford, England (1989); and in Stewart, J. M. Young, J. D. Solid-Phase Peptide Synthesis, 2nd Edition, Pierce Chemical Co., Rockford, IL (1984). The preferred strategy is based on the (9-fluorenylmethyloxycarbonyl) group (Fmoc) for temporary protection of the α-amino group, in combination with the tert-butyl group (tBu) for temporary protection of the amino acid side chains (see for example Atherton, E. et al, “The Fluorenylmethoxycarbonyl Amino Protecting Group”, in The Peptides: Analysis, Synthesis, Biology, Vol. 9: “Special Methods in Peptide Synthesis, Part C”, pp. 1-38, Undenfriend, S. et al, eds., Academic Press, San Diego (1987).

The peptides can be synthesized in a stepwise manner on an insoluble polymer support (also referred to as “resin”) starting from the C-terminus of the peptide. A synthesis is begun by appending the C-terminal amino acid of the peptide to the resin through formation of an amide or ester linkage. This allows the eventual release of the resulting peptide as a C-terminal amide or carboxylic acid, respectively.

The C-terminal amino acid and all other amino acids used in the synthesis are required to have their α-amino groups and side chain functionalities (if present) differentially protected such that the α-amino protecting group may be selectively removed during the synthesis. The coupling of an amino acid is performed by activation of its carboxyl group as an active ester and reaction thereof with the unblocked α-amino group of the N-terminal amino acid appended to the resin. The sequence of α-amino group deprotection and coupling is repeated until the entire peptide sequence is assembled. The peptide is then released from the resin with concomitant deprotection of the side chain functionalities, usually in the presence of appropriate scavengers to limit side reactions. The resulting peptide is finally purified by reverse phase HPLC.

The synthesis of the peptidyl-resins required as precursors to the final peptides utilizes commercially available cross-linked polystyrene polymer resins (Novabiochem, San Diego, CA; Applied Biosystems, Foster City, CA). Preferred solid supports are: 4-(2′,4′-dimethoxyphenyl-Fmoc-aminomethyl)-phenoxyacetyl-p-methyl benzhydrylamine resin (Rink amide MBHA resin); 9-Fmoc-amino-xanthen-3-yloxy-Merrifield resin (Sieber amide resin); 4-(9-Fmoc)aminomethyl-3,5-dimethoxyphenoxy)valerylaminomethyl-Merrifield resin (PAL resin), for C-terminal carboxamides. Coupling of first and subsequent amino acids can be accomplished using HOBt, 6-C1-HOBt or HOAt active esters produced from DIC/HOBt, HBTU/HOBt, BOP, PyBOP, or from DIC/6-C1-HOBt, HCTU, DIC/HOAt or HATU, respectively. Preferred solid supports are: 2-chlorotrityl chloride resin and 9-Fmoc-amino-xanthen-3-yloxy-Merrifield resin (Sieber amide resin) for protected peptide fragments. Loading of the first amino acid onto the 2-chlorotrityl chloride resin is best achieved by reacting the Fmoc-protected amino acid with the resin in dichloromethane and DIEA. If necessary, a small amount of DMF may be added to solubilize the amino acid.

The syntheses of the peptide analogs described herein can be carried out by using a single or multi-channel peptide synthesizer, such as an CEM Liberty Microwave synthesizer, or a Protein Technologies, Inc. Prelude (6 channels) or Symphony (12 channels) or Symphony X (24 channels) synthesizer.

Useful Fmoc amino acids derivatives are shown below.

Examples of Orthogonally Protected Amino Acids used in Solid Phase Synthesis:

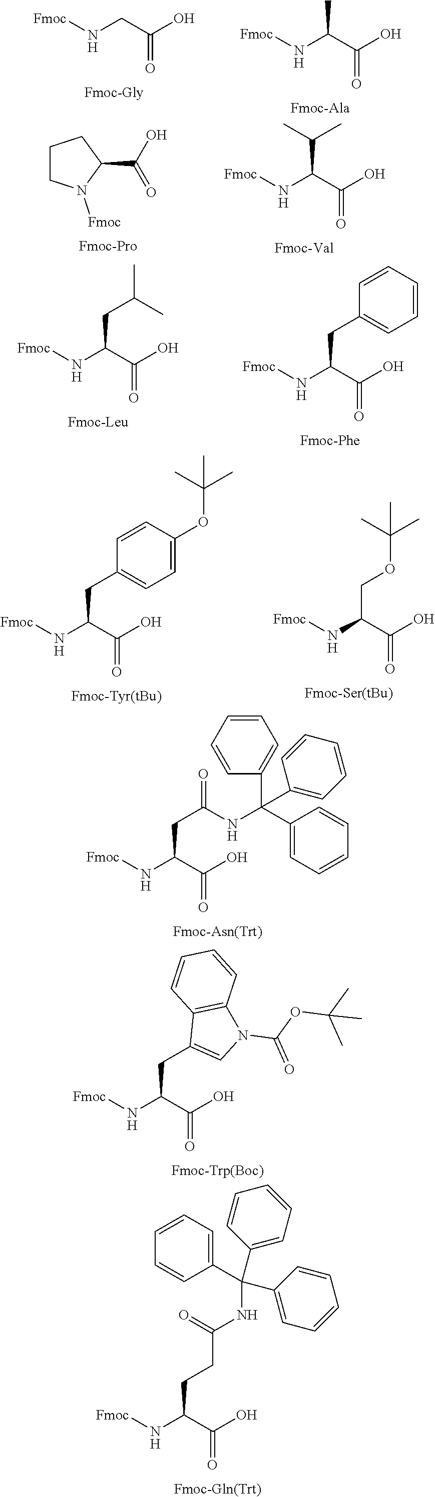

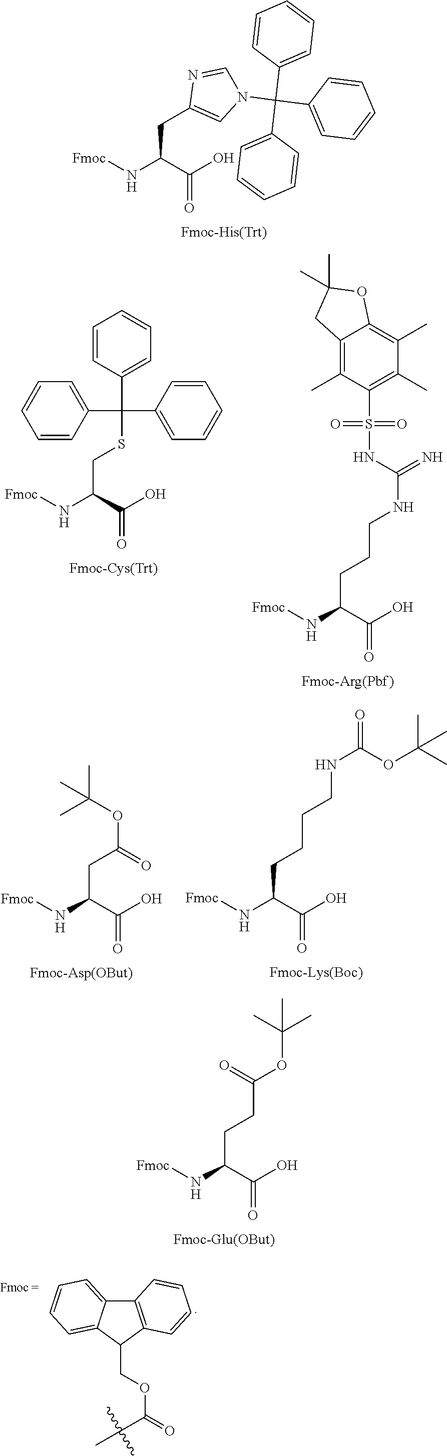

The peptidyl-resin precursors for their respective peptides may be cleaved and deprotected using any standard procedure (see, for example, King, D. S. et al, Int. J. Peptide Protein Res., 36:255-266 (1990)). A desired method is the use of TFA in the presence of TIS as scavenger and DTT or TCEP as the disulfide reducing agent. Typically, the peptidyl-resin is stirred in TFA/TIS/DTT (95:5:1 to 97:3:1), v:v:w; 1-3 mL/100 mg of peptidyl resin) for 1.5-3 hrs at room temperature. The spent resin is then filtered off and the TFA solution was cooled and Et₂O solution was added. The precipitates were collected by centrifuging and decanting the ether layer (3×). The resulting crude peptide is either redissolved directly into DMF or DMSO or CH₃CN/H₂O for purification by preparative HPLC or used directly in the next step.

Peptides with the desired purity can be obtained by purification using preparative HPLC, for example, on a Waters Model 4000 or a Shimadzu Model LC-8A liquid chromatography. The solution of crude peptide is injected into a YMC S5 ODS (20×100 mm) column and eluted with a linear gradient of MeCN in water, both buffered with 0.1% TFA, using a flow rate of 14-20 mL/min with effluent monitoring by UV absorbance at 217 or 220 nm. The structures of the purified peptides can be confirmed by electro-spray MS analysis.

List of unnatural amino acids referred to herein is provided below:

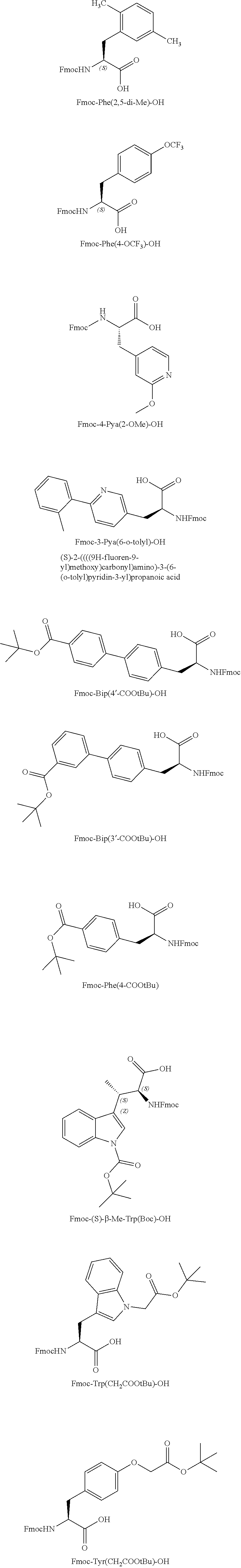

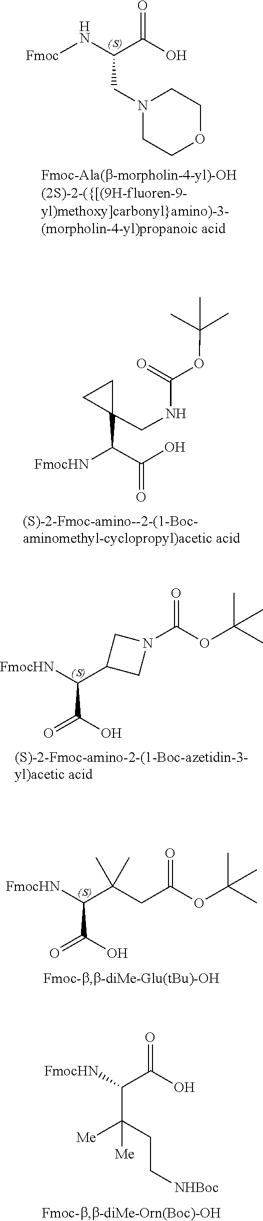

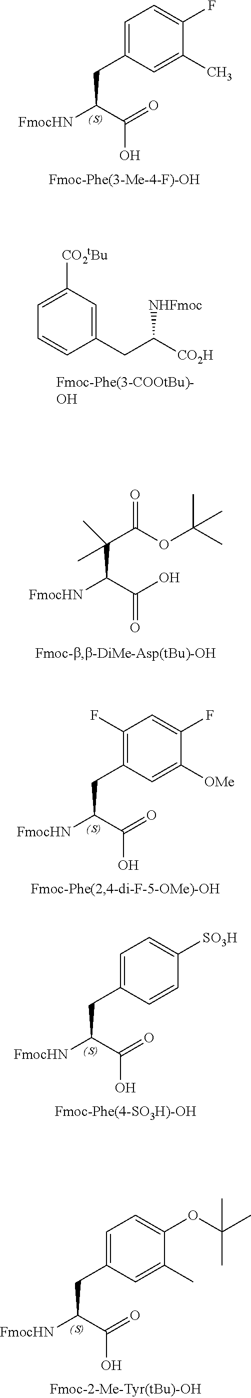

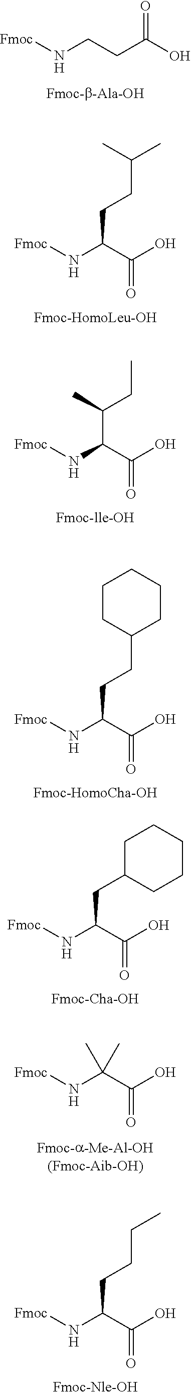

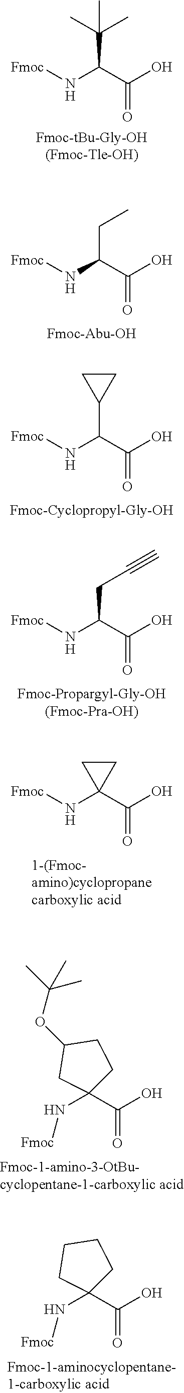

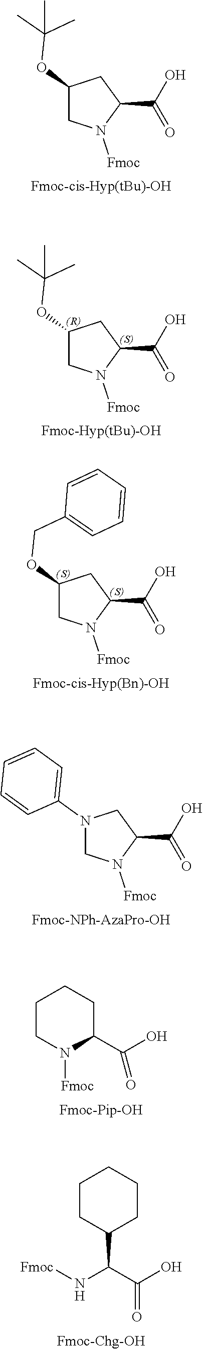

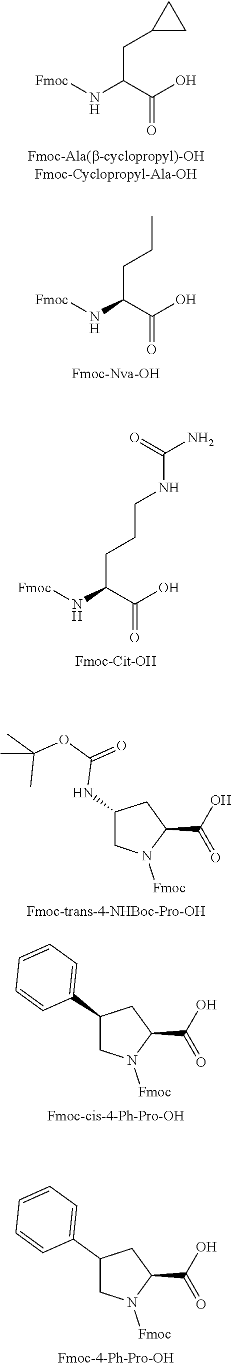

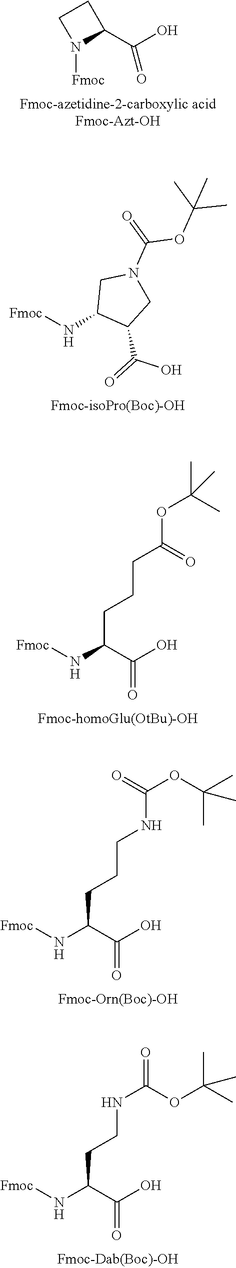

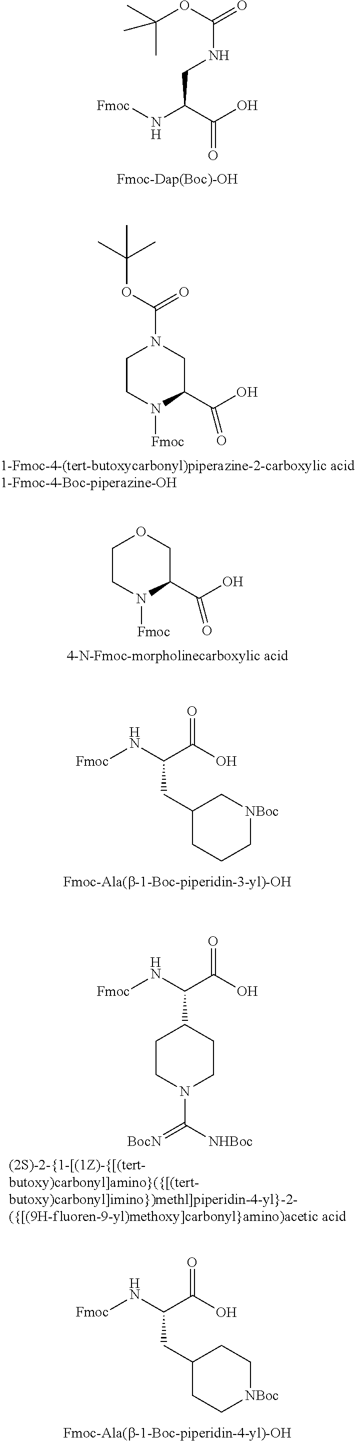

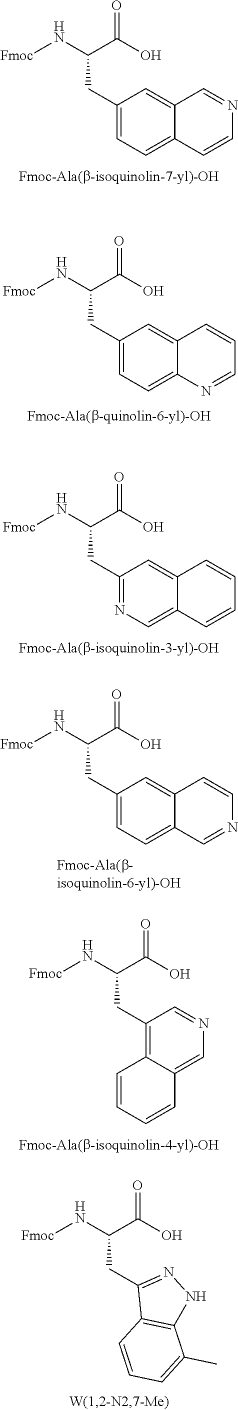

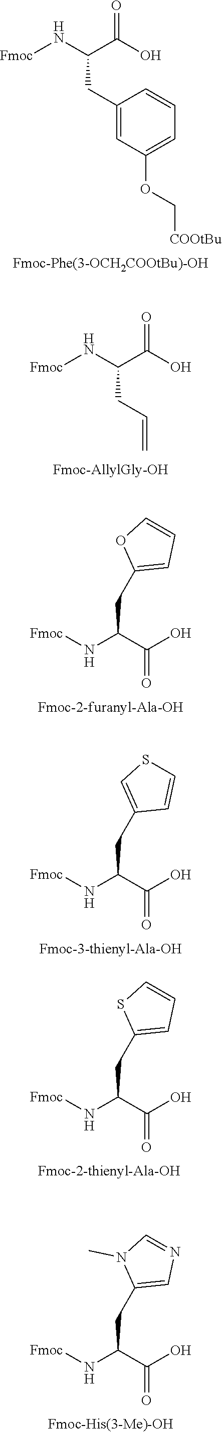

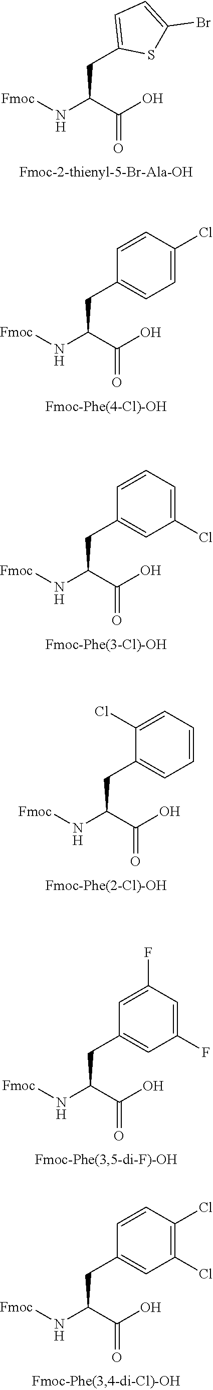

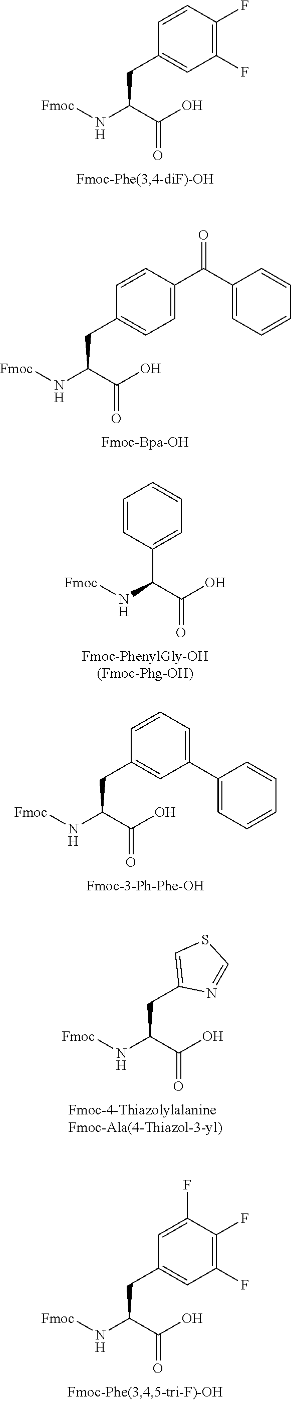

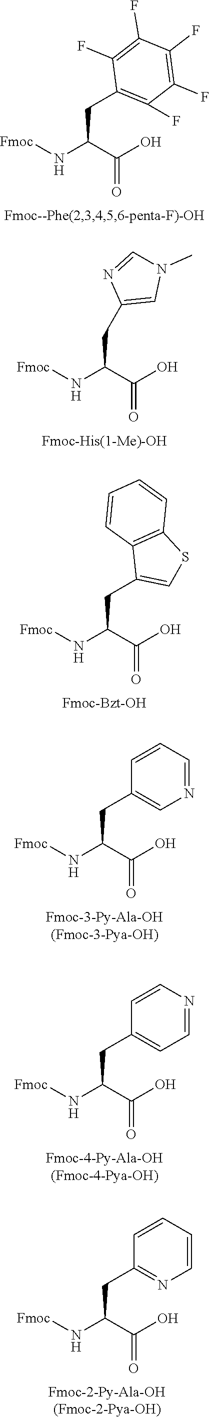

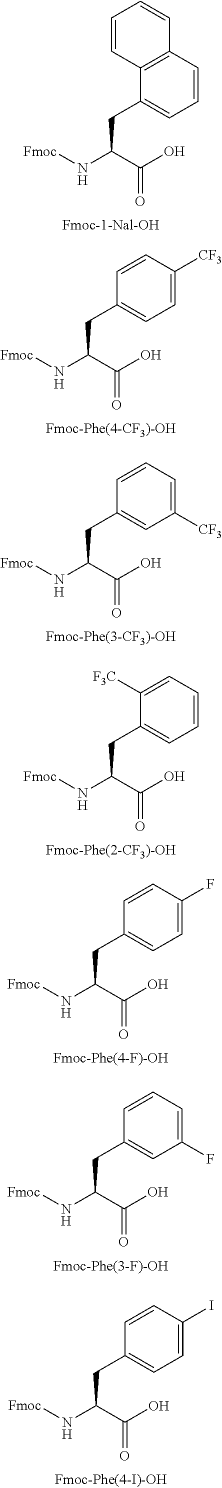

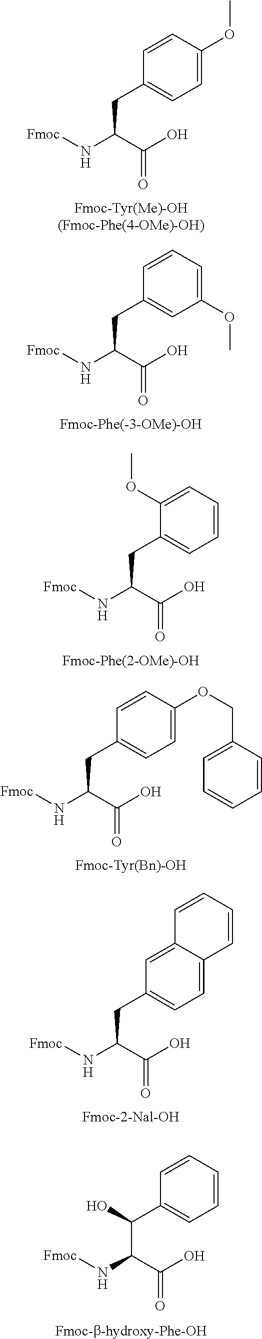

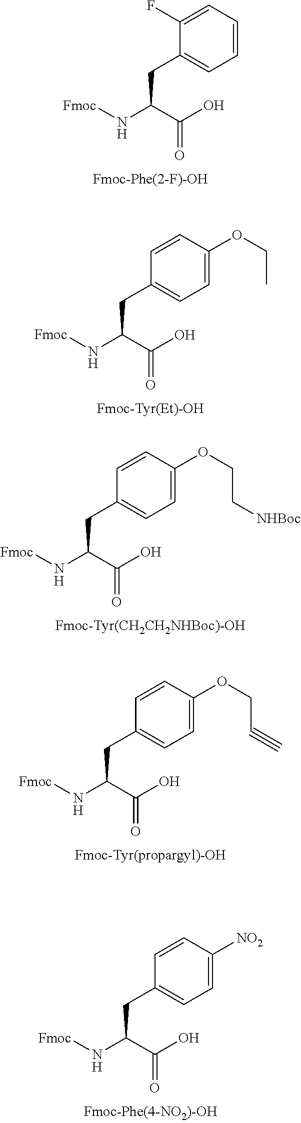

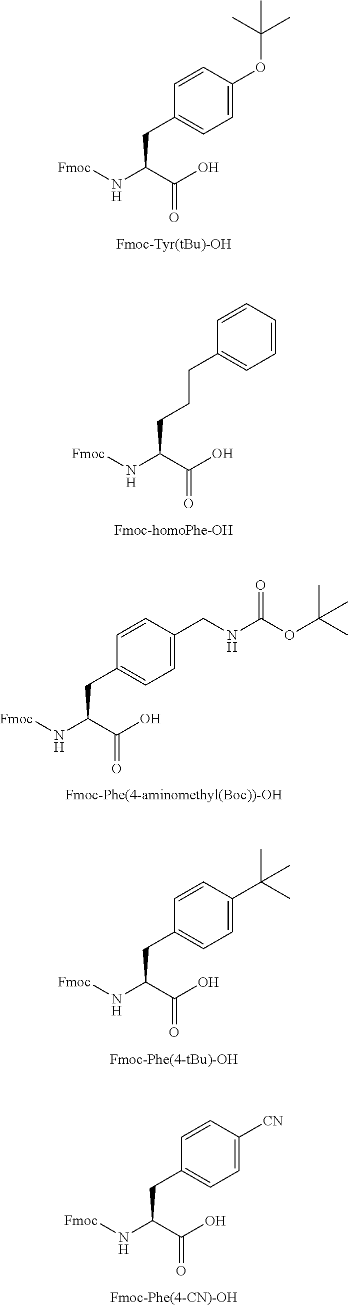

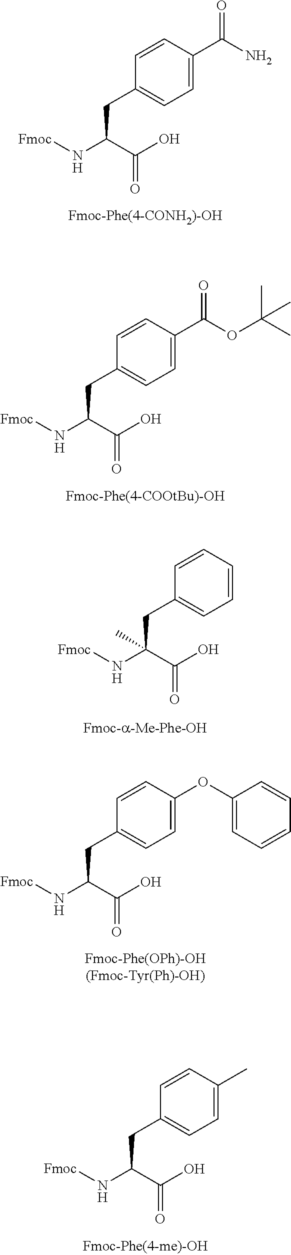

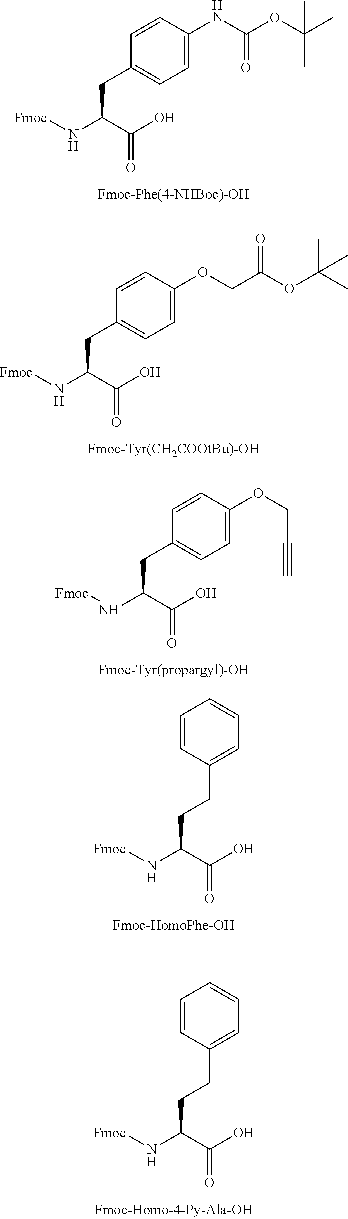

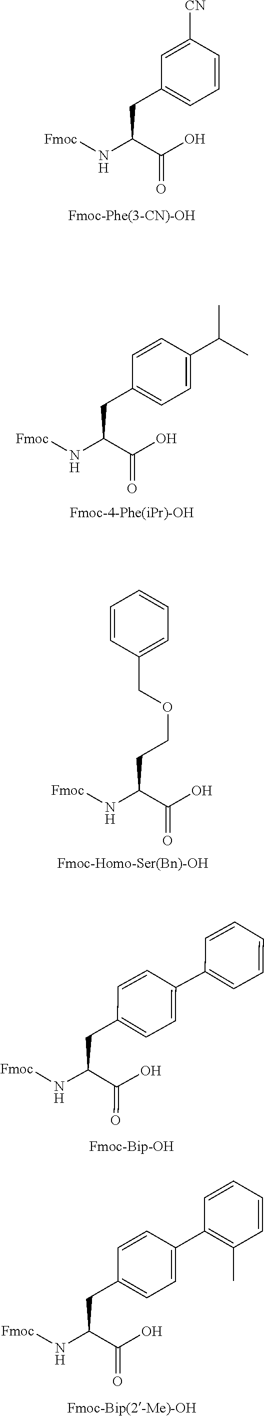

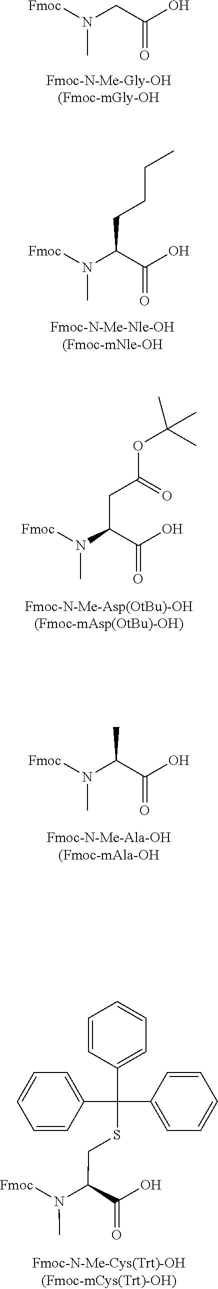

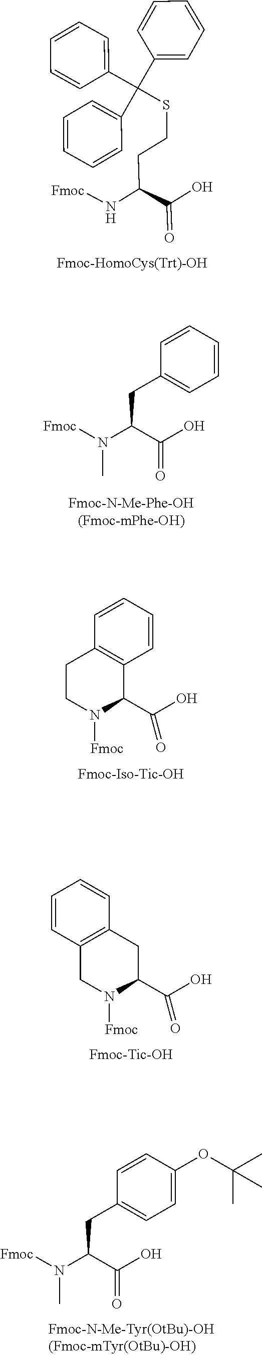

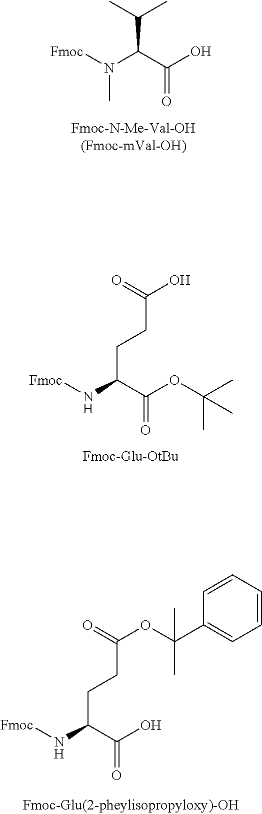

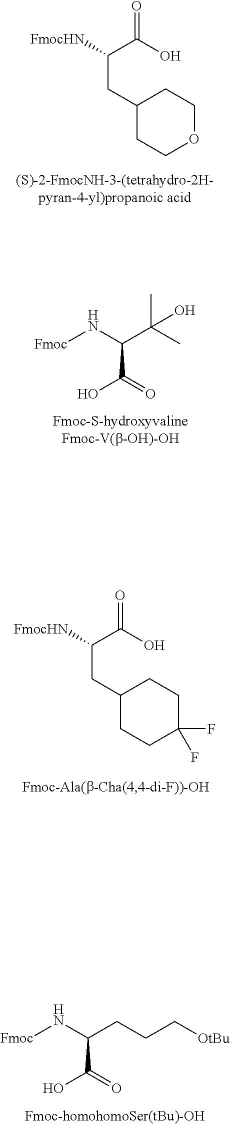

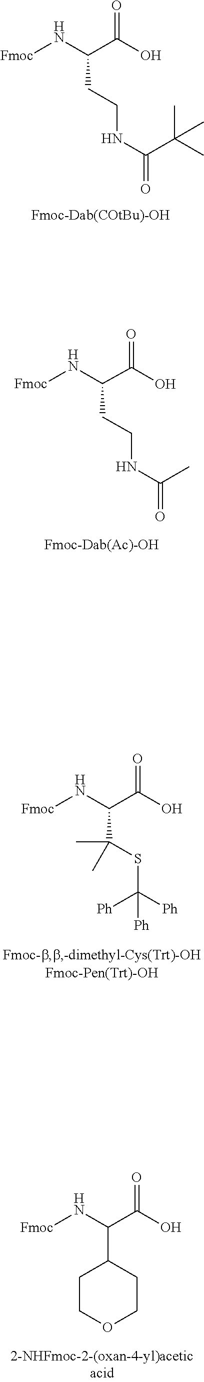

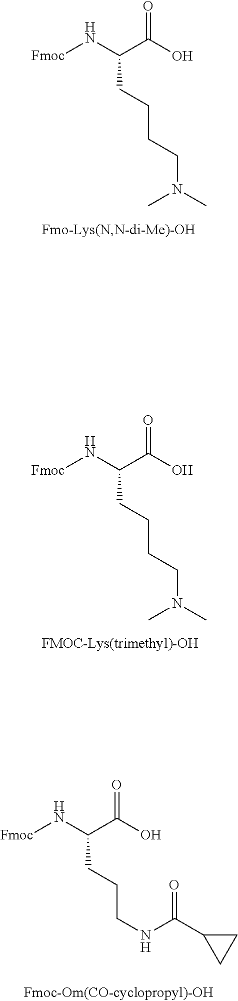

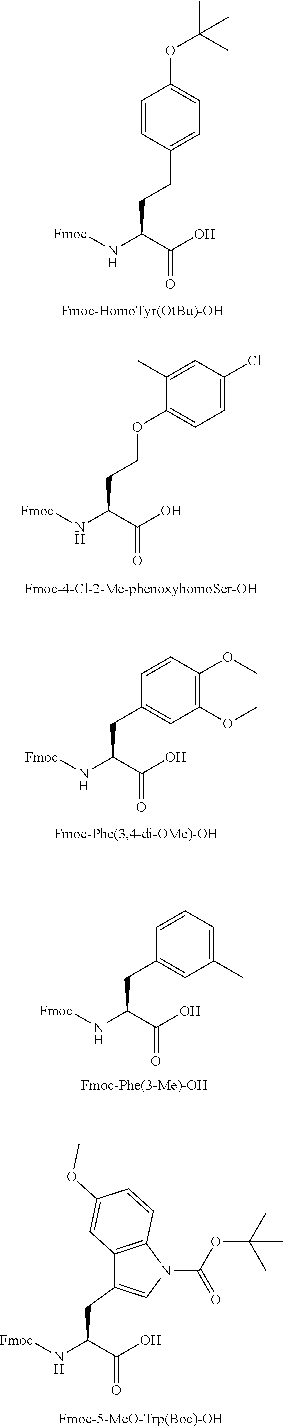

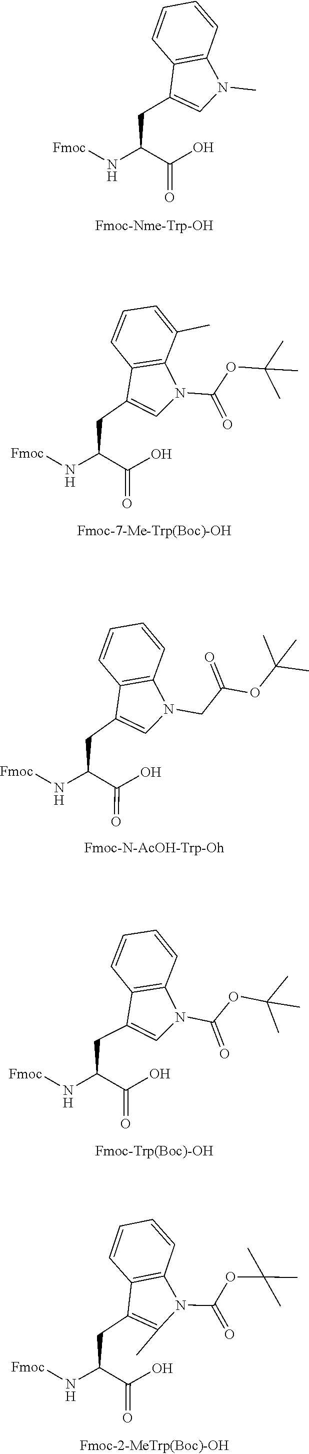

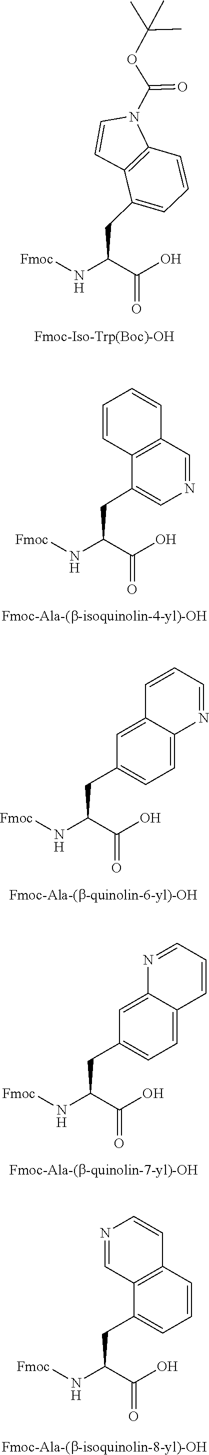

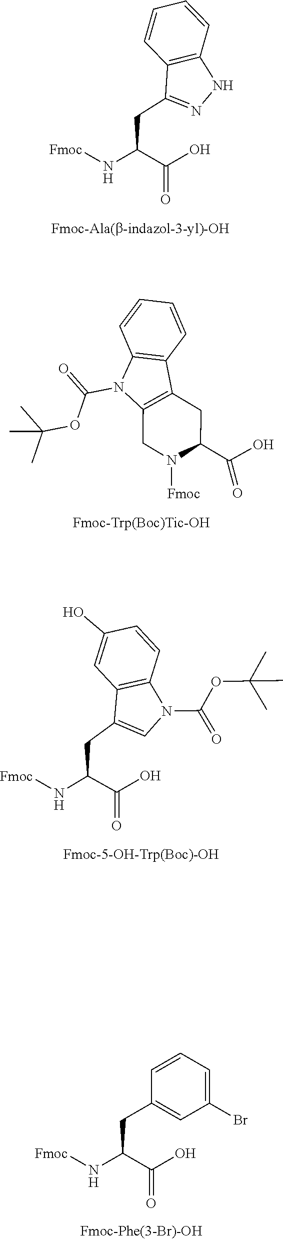

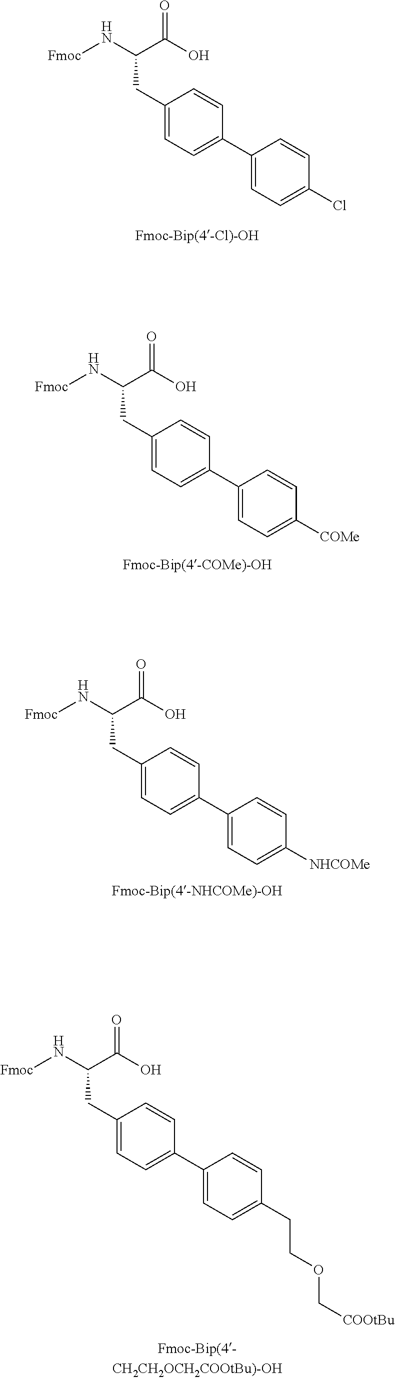

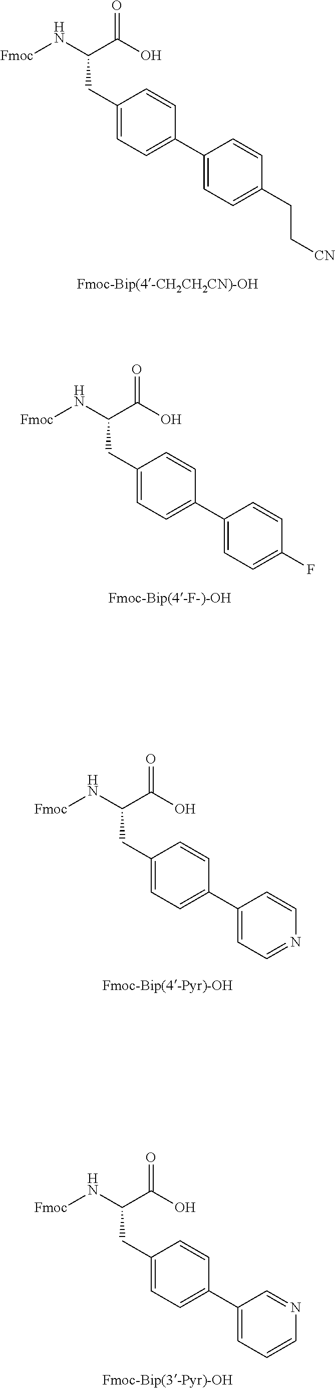

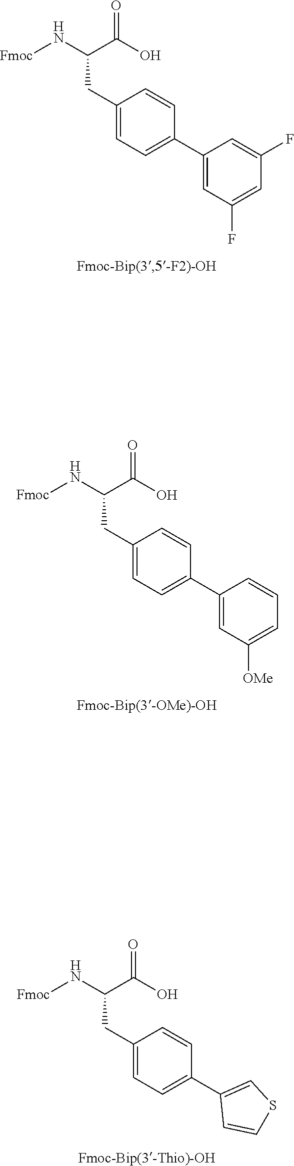

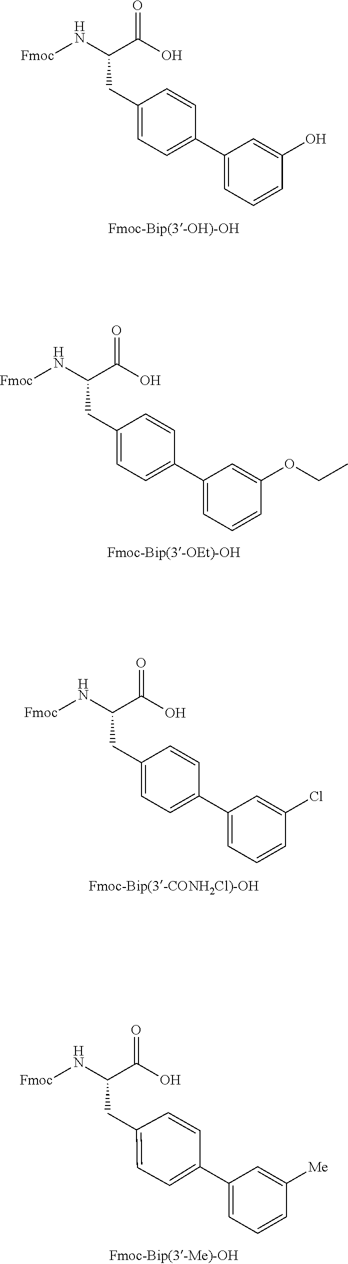

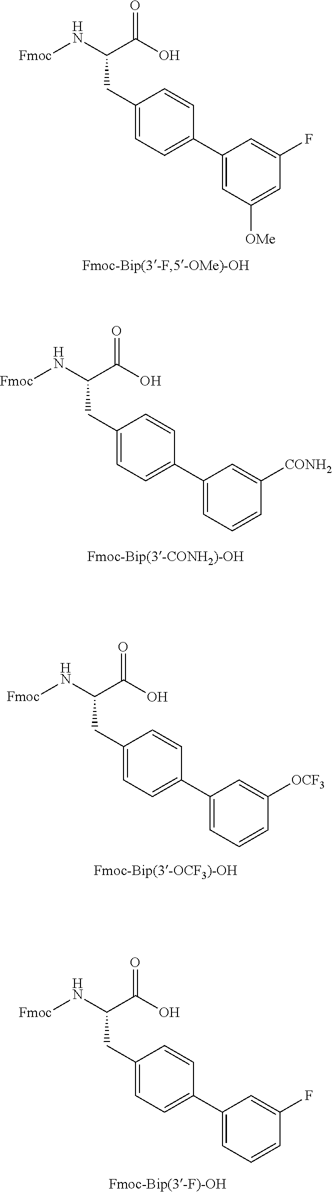

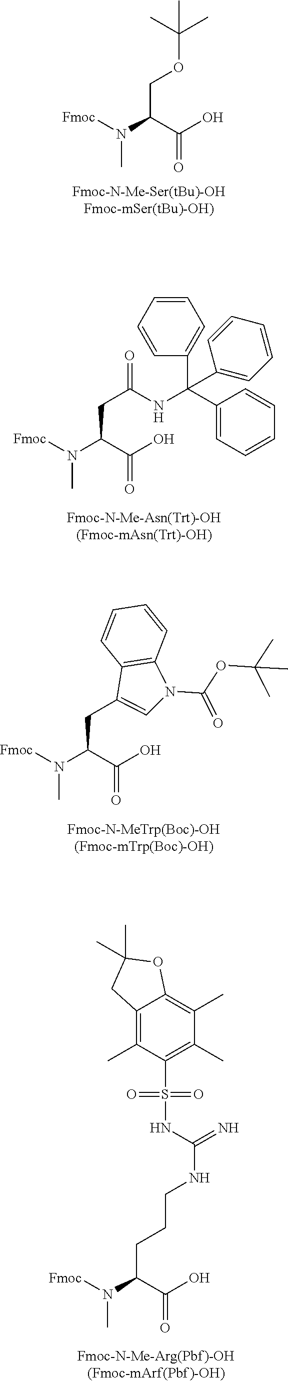

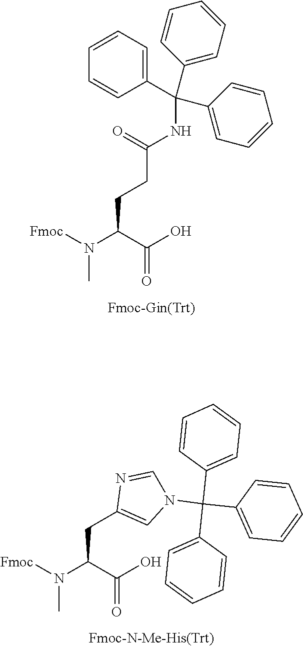

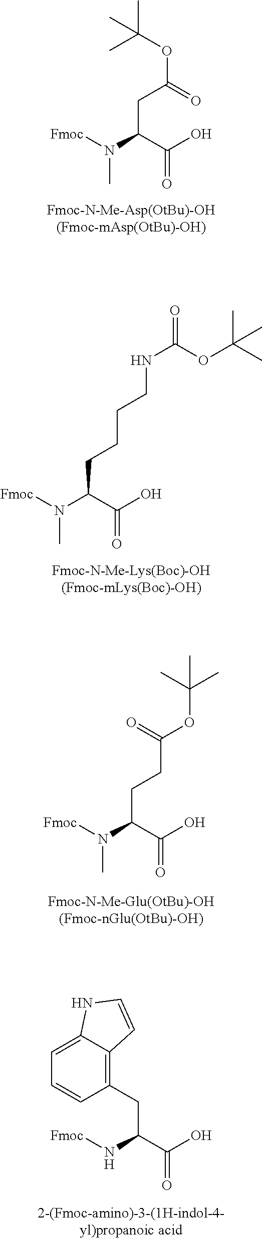

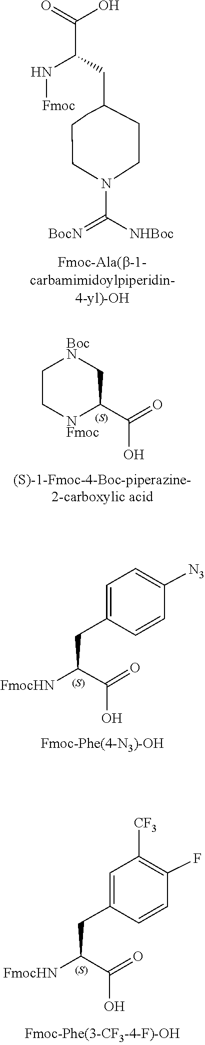

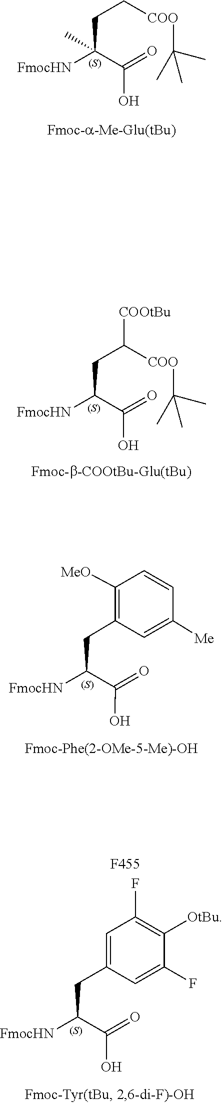

### General Analytical Protocols and Synthetic Methods

### Analytical Data

Mass Spectrometry: “ESI-MS(+)” signifies electrospray ionization mass spectrometry performed in positive ion mode; “ESI-MS(−)” signifies electrospray ionization mass spectrometry performed in negative ion mode; “ESI-HRMS(+)” signifies high-resolution electrospray ionization mass spectrometry performed in positive ion mode; “ESI-HRMS(−)” signifies high-resolution electrospray ionization mass spectrometry performed in negative ion mode. The detected masses are reported following the “m/z” unit designation. Compounds with exact masses greater than 1000 were often detected as double-charged or triple-charged ions. The crude material was purified via preparative LC/MS. Fractions containing the desired product were combined and dried via centrifugal evaporation.

### Analytical LCMS Condition A:

Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.75-minute hold at 100% B; Flow: 1.0 mL/min; Detection: UV at 220 nm.

### Analytical LC/MS Condition B:

Column: Waters Acquity UPLC BEH C18, 2.1×50 mm, 1.7-μm particles; Mobile Phase A: 5:95 acetonitrile:water with 0.1% trifluoroacetic acid; Mobile Phase B: 95:5 acetonitrile:water with 0.1% trifluoroacetic acid; Temperature: 50° C.; Gradient: 0-100% B over 3 minutes, then a 0.75-minute hold at 100% B; Flow: 1.0 mL/min; Detection: UV at 220 nm.

### Analytical LCMS Condition C:

Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 70° C.; Gradient: 0-100% B over 3 minutes, then a 2.0-minute hold at 100% B; Flow: 0.75 mL/min; Detection: UV at 220 nm.

### Analytical LCMS Condition D:

Column: Waters Acquity UPLC BEH C18, 2.1×50 mm, 1.7-μm particles; Mobile Phase A: 5:95 acetonitrile:water with 0.1% trifluoroacetic acid; Mobile Phase B: 95:5 acetonitrile:water with 0.1% trifluoroacetic acid; Temperature: 70° C.; Gradient: 0-100% B over 3 minutes, then a 2.0-minute hold at 100% B; Flow: 0.75 mL/min; Detection: UV at 220 nm.

### Analytical LC/MS Condition E:

Column: Kinetex XB C18, 3.0×75 mm, 2.6-μm particles; Mobile Phase A: 10 mM ammonium formate in water:acetonitrile (98:2); Mobile Phase B: 10 mM ammonium formate in Water:acetonitrile (02:98); Gradient: 20-100% B over 4 minutes, then a 0.6-minute hold at 100% B; Flow: 1.0 mL/min; Detection: UV at 254 nm.

### Analytical LC/MS Condition F:

Column: Ascentis Express C18, 2.1×50 mm, 2.7-μm particles; Mobile Phase A: 10 mM ammonium acetate in water:acetonitrile (95:5); Mobile Phase B: 10 mM ammonium acetate in Water:acetonitrile (05:95), Temperature: 50° C.; Gradient: 0-100% B over 3 minutes; Flow: 1.0 mL/min; Detection: UV at 220 nm.

### Analytical LC/MS Condition G:

Column: X Bridge C18, 4.6×50 mm, 5-μm particles; Mobile Phase A: 0.1% TFA in water; Mobile Phase B: acetonitrile, Temperature: 35° C.; Gradient: 5-95% B over 4 minutes; Flow: 4.0 mL/min; Detection: UV at 220 nm.

### Analytical LC/MS Condition H:

Column: X Bridge C18, 4.6×50 mm, 5-μm particles; Mobile Phase A: 10 mM NH₄OAc; Mobile Phase B: methanol, Temperature: 35° C.; Gradient: 5-95% B over 4 minutes; Flow: 4.0 mL/min; Detection: UV at 220 nm.

### Analytical LC/MS Condition I:

Column: X Bridge C18, 4.6×50 mm, 5-μm particles; Mobile Phase A: 10 mM NH₄₀Ac; Mobile Phase B: acetonitrile, Temperature: 35° C.; Gradient: 5-95% B over 4 minutes; Flow: 4.0 mL/min; Detection: UV at 220 nm.

### Analytical LC/MS Condition J:

Column: Waters Acquity UPLC BEH C18, 2.1×50 mm, 1.7-μm particles; Mobile Phase A: 5:95 acetonitrile:water with 0.05% trifluoroacetic acid; Mobile Phase B: 95:5 acetonitrile:water with 0.05% trifluoroacetic acid; Temperature: 70° C.; Gradient: 0-100% B over 1.5 minutes, then a 2.0-minute hold at 100% B; Flow: 0.75 mL/min; Detection: UV at 254 nm.

### Analytical LCMS Condition K:

Column: Waters Acquity UPLC BEH C18, 2.1×50 mm, 1.7-μm particles; Mobile Phase A: 100% water with 0.05% trifluoroacetic acid; Mobile Phase B: 100% acetonitrile with 0.05% trifluoroacetic acid; Temperature: 50° C.; Gradient: 2-98% B over 1.0 minutes, then at 1.0-1.5 minute hold at 98% B; Flow: 0.80 mL/min; Detection: UV at 220 nm.

### Analytical LCMS Condition L:

Column: Waters Acquity UPLC BEH C18, 2.1×50 mm, 1.7-μm particles; Buffer: 10 mM Ammonium Acetate. Mobile Phase A: buffer” CH₃CN (95/5); Mobile Phase B: Mobile Phase B:Buffer:ACN(5:95); Temperature: 50° C.; Gradient: 20-98% B over 2.0 minutes, then at 0.2 minute hold at 100% B; Flow: 0.70 mL/min; Detection: UV at 220 nm.

### Analytical LCMS Condition M:

Column: Waters Acquity UPLC BEH C18, 3.0×50 mm, 1.7-μm particles; Mobile Phase A: 95% water and 5% water with 0.1% trifluoroacetic acid; Mobile Phase B: 95% acetonitrile and 5% water with 0.1% trifluoroacetic acid; Temperature: 50° C.; Gradient: 20-100% B over 2.0 minutes, then at 2.0-2.3 minute hold at 100% B; Flow: 0.7 mL/min; Detection: UV at 220 nm.

### Analytical LCMS Condition N:

Column: Waters Acquity UPLC BEH C18, 2.1×50 mm, 1.7-μm particles; Mobile Phase A: 100% water with 0.05% trifluoroacetic acid; Mobile Phase B: 100% acetonitrile with 0.05% trifluoroacetic acid; Temperature: 50° C.; Gradient: 2-98% B over 5.0 minutes, then at 5.0-5.5 minute hold at 98% B; Flow: 0.80 mL/min; Detection: UV at 220 nm.

### Analytical LCMS Condition O:

Column: Waters Acquity UPLC BEH C18, 2.1×50 mm, 1.7-μm particles; Mobile Phase A: 5:95 acetonitrile:water with 0.05% trifluoroacetic acid; Mobile Phase B: 95:5 acetonitrile:water with 0.05% trifluoroacetic acid; Temperature: 50° C.; Gradient: 2%-98% B over 2 minutes, then a 0.5-minute hold at 98% B; Flow: 0.8 mL/min; Detection: UV at 220 nm.

### Analytical LC/MS Condition P:

Column: Waters Acquity UPLC BEH C18, 2.1×50 mm, 1.7-μm particles; Mobile Phase A: 5:95 acetonitrile:water with 0.05% trifluoroacetic acid; Mobile Phase B: 95:5 acetonitrile:water with 0.05% trifluoroacetic acid; Temperature: 50° C.; Gradient: 0%-100% B over 3 minutes, then a 0.5-minute hold at 100% B; Flow: 1.0 mL/min; Detection: UV at 220 nm.

### Analytical LC/MS Condition Q:

Column: Waters Acquity UPLC BEH C18, 2.1×50 mm, 1.7-μm particles; Mobile Phase A: 5:95 acetonitrile:water with 0.05% trifluoroacetic acid; Mobile Phase B: 95:5 acetonitrile:water with 0.05% trifluoroacetic acid; Temperature: 50° C.; Gradient: 0%-100% B over 1 minute, then a 0.5-minute hold at 100% B; Flow: 1.0 mL/min; Detection: UV at 220 nm.

### Analytical LC/MS Condition R:

Column: Waters Acquity UPLC BEH C18, 2.1×50 mm, 1.7-μm particles; Buffer:10 mM Ammonium Acetate. Mobile Phase A: buffer” CH₃CN (95/5); Mobile Phase B: Mobile Phase B:Buffer:ACN(5:95); Temperature: 50° C.; Gradient: 0%-100% B over 1 minute, then a 0.5-minute hold at 100% B; Flow: 1.0 mL/min; Detection: UV at 220 nm.

### Analytical LC/MS Condition S:

Column: Waters Acquity UPLC BEH C18, 2.1×50 mm, 1.7-μm particles; Mobile Phase A: 100% water with 0.05% trifluoroacetic acid; Mobile Phase B: 100% acetonitrile with 0.05% trifluoroacetic acid; Gradient: 2-98% B over 1.6 minutes, then at 0.2 minute hold at 98% B; Flow: 0.80 mL/min; Detection: UV at 220 nm.

### Analytical LCMS Condition T:

Column: Waters Acquity UPLC BEH C18, 2.1×50 mm, 1.7-μm particles; Mobile Phase A: 5:95 acetonitrile:water with 0.05% trifluoroacetic acid; Mobile Phase B: 95:5 acetonitrile:water with 0.05% trifluoroacetic acid; Gradient: 2%-98% B over 2.6 minutes, then a 0.4-minute hold at 98% B; Flow: 0.8 mL/min; Detection: UV at 220 nm.

### Analytical LC/MS Condition U.

Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Gradient: 30-100% B over 3 minutes, then a 0.75-minute hold at 100% B; Flow: 1.0 mL/min; Detection: UV at 220 nm.

### General Procedures

### Prelude Method

All manipulations were performed under automation on a Prelude peptide synthesizer (Protein Technologies). Unless noted, all procedures were performed in a 45-mL polypropylene reaction vessel fitted with a bottom frit. The reaction vessel connects to the Prelude peptide synthesizer through both the bottom and the top of the vessel. DMF and DCM can be added through the top of the vessel, which washes down the sides of the vessel equally. The remaining reagents are added through the bottom of the reaction vessel and pass up through the frit to contact the resin. All solutions are removed through the bottom of the reaction vessel. “Periodic agitation” describes a brief pulse of N₂ gas through the bottom frit; the pulse lasts approximately 5 seconds and occurs every 30 seconds. Amino acid solutions were generally not used beyond two weeks from preparation. HATU solution was used within 7-14 days of preparation.

Sieber amide resin=9-Fmoc-aminoxanthen-3-yloxy polystyrene resin, where “3-yloxy” describes the position and type of connectivity to the polystyrene resin. The resin used is polystyrene with a Sieber linker (Fmoc-protected at nitrogen); 100-200 mesh, 1% DVB, 0.71 mmol/g loading.

Rink=(2,4-dimethoxyphenyl)(4-alkoxyphenyl)methanamine, where “4-alkoxy” describes the position and type of connectivity to the polystyrene resin. The resin used is Merrifield polymer (polystyrene) with a Rink linker (Fmoc-protected at nitrogen); 100-200 mesh, 1% DVB, 0.56 mmol/g loading.

2-Chlorotrityl chloride resin (2-Chlorotriphenylmethyl chloride resin), 50-150 mesh, 1% DVB, 1.54 mmol/g loading. Fmoc-glycine-2-chlorotrityl chloride resin, 200-400 mesh, 1% DVB, 0.63 mmol/g loading.

PL-FMP resin: (4-Formyl-3-methoxyphenoxymethyl)polystyrene.

Common amino acids used are listed below with side-chain protecting groups indicated inside parenthesis.

Fmoc-Ala-OH; Fmoc-Arg(Pbf)-OH; Fmoc-Asn(Trt)-OH; Fmoc-Asp(tBu)-OH; Fmoc-Bip-OH; Fmoc-Cys(Trt)-OH; Fmoc-Dab(Boc)-OH; Fmoc-Dap(Boc)-OH; Fmoc-Gln(Trt)-OH; Fmoc-Gly-OH; Fmoc-His(Trt)-OH; Fmoc-Hyp(tBu)-OH; Fmoc-Ile-OH; Fmoc-Leu-OH; Fmoc-Lys(Boc)-OH; Fmoc-Nle-OH; Fmoc-Met-OH; Fmoc-[N-Me]Ala-OH; Fmoc-[N-Me]Nle-OH; Fmoc-Orn(Boc)-OH, Fmoc-Phe-OH; Fmoc-Pro-OH; Fmoc-Sar-OH: Fmoc-Ser(tBu)-OH; Fmoc-Thr(tBu)-OH; Fmoc-Trp(Boc)-OH; Fmoc-Tyr(tBu)-OH; Fmoc-Val-OH and their corresponding D-amino acids.

The procedures of “Prelude Method” describe an experiment performed on a 0.100 mmol scale, where the scale is determined by the amount of Sieber or Rink or 2-chlorotrityl or PL-FMP resin. This scale corresponds to approximately 140 mg of the Sieber amide resin described above. All procedures can be scaled down or up from the 0.100 mmol scale by adjusting the described volumes by the multiple of the scale. Prior to amino acid coupling, all peptide synthesis sequences began with a resin-swelling procedure, described below as “Resin-swelling procedure”. Coupling of amino acids to a primary amine N-terminus used the “Single-coupling procedure” described below. Coupling of amino acids to a secondary amine N-terminus or to the N-terminus of Arg(Pbf)- and D-Arg(Pbf)- used the “Double-coupling procedure” described below.

### Resin-Swelling Procedure:

To a 45-mL polypropylene solid-phase reaction vessel was added Sieber amide resin (140 mg, 0.100 mmol). The resin was washed (swelled) two times as follows: to the reaction vessel was added DMF (5.0 mL) through the top of the vessel “DMF top wash” upon which the mixture was periodically agitated for 10 minutes before the solvent was drained through the frit.

### Single-Coupling Procedure:

To the reaction vessel containing the resin from the previous step was added piperidine:DMF (20:80 v/v, 5.0 mL). The mixture was periodically agitated for 5.0 minutes and then the solution was drained through the frit. To the reaction vessel was added piperidine:DMF (20:80 v/v, 5.0 mL). The mixture was periodically agitated for 5.0 minutes and then the solution was drained through the frit. The resin was washed successively six times as follows: for each wash, DMF (6.0 mL) was added through the top of the vessel and the resulting mixture was periodically agitated for 1.0 minutes before the solution was drained through the frit. To the reaction vessel was added the amino acid (0.2 M in DMF, 5.0 mL, 10 equiv), then HATU (0.4 M in DMF, 2.5 mL, 10 equiv), and finally NMM (0.8 M in DMF, 2.5 mL, 20 equiv). The mixture was periodically agitated for 60-120 minutes, then the reaction solution was drained through the frit. The resin was washed successively four times as follows: for each wash, DMF (5.0 mL) was added through the top of the vessel and the resulting mixture was periodically agitated for 1.0 minute before the solution was drained through the frit. The resulting resin was used directly in the next step.

### Double-Coupling Procedure:

To the reaction vessel containing the resin from the previous step was added piperidine:DMF (20:80 v/v, 5.0 mL). The mixture was periodically agitated for 5.0 minutes and then the solution was drained through the frit. To the reaction vessel was added piperidine:DMF (20:80 v/v, 5.0 mL). The mixture was periodically agitated for 5.0 minutes and then the solution was drained through the frit. The resin was washed successively six times as follows: for each wash, DMF (6.0 mL) was added through the top of the vessel and the resulting mixture was periodically agitated for 1.0 minutes before the solution was drained through the frit. To the reaction vessel was added the amino acid (0.2 M in DMF, 5.0 mL, 10 equiv), then HATU (0.4 M in DMF, 2.5 mL, 10 equiv), and finally NMM (0.8 M in DMF, 2.5 mL, 20 equiv). The mixture was periodically agitated for 1-1.5 hour, then the reaction solution was drained through the frit. The resin was washed successively two times as follows: for each wash, DMF (5.0 mL) was added through the top of the vessel and the resulting mixture was periodically agitated for 1.0 minute before the solution was drained through the frit. To the reaction vessel was added the amino acid (0.2 M in DMF, 5.0 mL, 10 equiv), then HATU (0.4 M in DMF, 2.5 mL, 10 equiv), and finally NMM (0.8 M in DMF, 2.5 mL, 20 equiv). The mixture was periodically agitated for 1-1.5 hours, then the reaction solution was drained through the frit. The resin was washed successively four times as follows: for each wash, DMF (5.0 mL) was added through the top of the vessel and the resulting mixture was periodically agitated for 1.0 minute before the solution was drained through the frit. The resulting resin was used directly in the next step.

### Single-Coupling Manual Addition Procedure A:

To the reaction vessel containing the resin from the previous step was added piperidine:DMF (20:80 v/v, 5.0 mL). The mixture was periodically agitated for 5 minutes and then the solution was drained through the frit. To the reaction vessel was added piperidine:DMF (20:80 v/v, 5.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. The reaction was paused. The reaction vessel was opened and the unnatural amino acid (2-4 equiv) in DMF (1-2 mL) was added manually using a pipette from the top of the vessel while the bottom of the vessel was remain attached to the instrument, then the vessel was closed. The automatic program was resumed and HATU (0.4 M in DMF, 1.3 mL, 4 equiv) and NMM (1.3 M in DMF, 1.0 mL, 8 equiv) were added sequentially. The mixture was periodically agitated for 2-3 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Single-Coupling Manual Addition Procedure B:

To the reaction vessel containing the resin from the previous step was added piperidine:DMF (20:80 v/v, 5.0 mL). The mixture was periodically agitated for 5 minutes and then the solution was drained through the frit. To the reaction vessel was added piperidine:DMF (20:80 v/v, 5.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. The reaction was paused. The reaction vessel was opened and the unnatural amino acid (2-4 equiv) in DMF (1-1.5 mL) was added manually using a pipette from the top of the vessel while the bottom of the vessel was remain attached to the instrument, followed by the manual addition of HATU (2-4 equiv, same equiv as the unnatural amino acid), and then the vessel was closed. The automatic program was resumed and NMM (1.3 M in DMF, 1.0 mL, 8 equiv) were added sequentially. The mixture was periodically agitated for 2-3 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

Peptoid Installation (50 μmol) Procedure:

To the reaction vessel containing the resin from the previous step was added piperidine: DMF (20:80 v/v, 4.0 mL). The mixture was periodically agitated for 5 minutes and then the solution was drained through the frit. To the reaction vessel was added piperidine: DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 60 seconds before the solution was drained through the frit. To the reaction vessel was added bromoacetic acid (0.4 M in DMF, 2.0 mL, 16 eq), then DIC (0.4 M in DMF, 2.0 mL, 16 eq). The mixture was periodically agitated for 1 hour, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. To the reaction vessel was added the amine (0.4 M in DMF, 2.0 mL, 16 eq). The mixture was periodically agitated for 1 hour, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Chloroacetic Anhydride Coupling.

To the reaction vessel containing the resin from the previous step was added piperidine:DMF (20:80 v/v, 5.0 mL). The mixture was periodically agitated for 5 minutes and then the solution was drained through the frit. To the reaction vessel was added piperidine:DMF (20:80 v/v, 5.0 mL). The mixture was periodically agitated for 5 minutes and then the solution was drained through the frit. The resin was washed successively six times as follows: for each wash, DMF (6.0 mL) was added through the top of the vessel and the resulting mixture was periodically agitated for one minute before the solution was drained through the frit. To the reaction vessel was added the chloroacetic anhydride solution (0.4 M in DMF, 5.0 mL, 20 equiv), then N-methylmorpholine (0.8 M in DMF, 5.0 mL, 40 equiv). The mixture was periodically agitated for 15 minutes, then the reaction solution was drained through the frit. The resin was washed twice as follows: for each wash, DMF (6.0 mL) was added through the top of the vessel and the resulting mixture was periodically agitated for one minute before the solution was drained through the frit. To the reaction vessel was added the chloroacetic anhydride solution (0.4 M in DMF, 5.0 mL, 20 equiv), then N-methylmorpholine (0.8 M in DMF, 5.0 mL, 40 equiv). The mixture was periodically agitated for 15 minutes, then the reaction solution was drained through the frit. The resin was washed successively five times as follows: for each wash, DMF (6.0 mL) was added through the top of the vessel and the resulting mixture was periodically agitated for one minute before the solution was drained through the frit. The resin was washed successively four times as follows: for each wash, DCM (6.0 mL) was added through the top of the vessel and the resulting mixture was periodically agitated for one minute before the solution was drained through the frit. The resin was then dried with nitrogen flow for 10 minutes. The resulting resin was used directly in the next step.

### Symphony Method:

All manipulations were performed under automation on a 12-channel Symphony peptide synthesizer (Protein Technologies). Unless noted, all procedures were performed in a 25-mL polypropylene reaction vessel fitted with a bottom frit. The reaction vessel connects to the Symphony peptide synthesizer through both the bottom and the top of the vessel. DMF and DCM can be added through the top of the vessel, which washes down the sides of the vessel equally. The remaining reagents are added through the bottom of the reaction vessel and pass up through the frit to contact the resin. All solutions are removed through the bottom of the reaction vessel. “Periodic agitation” describes a brief pulse of N₂ gas through the bottom frit; the pulse lasts approximately 5 seconds and occurs every 30 seconds. Amino acid solutions were generally not used beyond two weeks from preparation. HATU solution were used within 7-14 days of preparation.

Sieber amide resin=9-Fmoc-aminoxanthen-3-yloxy polystyrene resin, where “3-yloxy” describes the position and type of connectivity to the polystyrene resin. The resin used is polystyrene with a Sieber linker (Fmoc-protected at nitrogen); 100-200 mesh, 1% DVB, 0.71 mmol/g loading.

Rink=(2,4-dimethoxyphenyl)(4-alkoxyphenyl)methanamine, where “4-alkoxy” describes the position and type of connectivity to the polystyrene resin. The resin used is Merrifield polymer (polystyrene) with a Rink linker (Fmoc-protected at nitrogen); 100-200 mesh, 1% DVB, 0.56 mmol/g loading.

2-Chlorotrityl chloride resin (2-Chlorotriphenylmethyl chloride resin), 50-150 mesh, 1% DVB, 1.54 mmol/g loading.

PL-FMP resin: (4-Formyl-3-methoxyphenoxymethyl)polystyrene.

Fmoc-glycine-2-chlorotrityl chloride resin, 200-400 mesh, 1% DVB, 0.63 mmol/g loading.

Common amino acids used are listed below with side-chain protecting groups indicated inside parenthesis:
Fmoc-Ala-OH; Fmoc-Arg(Pbf)-OH; Fmoc-Asn(Trt)-OH; Fmoc-Asp(tBu)-OH; Fmoc-Bip-OH; Fmoc-Cys(Trt)-OH; Fmoc-Dab(Boc)-OH; Fmoc-Dap(Boc)-OH; Fmoc-Gln(Trt)-OH; Fmoc-Gly-OH Fmoc-Gly-OH; Fmoc-His(Trt)-OH; Fmoc-Hyp(tBu)-OH; Fmoc-Ile-OH; Fmoc-Leu-OH; Fmoc-Lys(Boc)-OH; Fmoc-Nle-OH; Fmoc-Met-OH; Fmoc-[N-Me]Ala-OH; Fmoc-[N-Me]Nle-OH; Fmoc-Orn(Boc)-OH, Fmoc-Phe-OH; Fmoc-Pro-OH; Fmoc-Sar-OH; Fmoc-Ser(tBu)-OH; Fmoc-Thr(tBu)-OH; Fmoc-Trp(Boc)-OH; Fmoc-Tyr(tBu)-OH; Fmoc-Val-OH and their corresponding D-amino acids.

The procedures of “Symphony Method” describe an experiment performed on a 0.05 mmol scale, where the scale is determined by the amount of Sieber or Rink or chlorotrityl linker or PL-FMP bound to the resin. This scale corresponds to approximately 70 mg of the Sieber resin described above. All procedures can be scaled up from the 0.05 mmol scale by adjusting the described volumes by the multiple of the scale.

Prior to the amino acid coupling, all peptide synthesis sequences began with a resin-swelling procedure, described below as “Resin-swelling procedure”. Coupling of amino acids to a primary amine N-terminus used the “Single-coupling procedure” described below. Coupling of amino acids to a secondary amine N-terminus or to the N-terminus of Arg(Pbf)- and D-Arg(Pbf)- used the “Double-coupling procedure” described below.

### Resin-Swelling Procedure:

To a 25-mL polypropylene solid-phase reaction vessel was added the resin (0.05 mmol). The resin was washed (swelled) as follows: to the reaction vessel was added DMF (2.0-3.0 mL, 1-2 times), upon which the mixture was periodically agitated for 10 minutes before the solvent was drained through the frit. Sometimes the resin was washed (swelled) as follows: to the reaction vessel was added CH₂Cl₂ (3-5 mL, 2 times) upon which the mixture was periodically agitated for 30 min before the solvent was drained through the frit. Then DMF (2.0-3.0 mL, 1-6 times), was added upon which the mixture was periodically agitated for 2-10 minutes before the solvent was drained through the frit.

### Single-Coupling Procedure:

To the reaction vessel containing the resin from the previous step was added DMF (2.5-3.75 mL) three times, upon which the mixture was agitated for 30 seconds before the solvent was drained through the frit each time. To the resin was added piperidine:DMF (20:80 v/v, 3.0-3.75 mL). The mixture was periodically agitated for 5.0 minutes and then the solution was drained through the frit. To the reaction vessel was added piperidine:DMF (20:80 v/v, 3.0-3.75 mL). The mixture was periodically agitated for 5.0 minutes and then the solution was drained through the frit. Sometimes the deprotection step was performed a third time. The resin was washed successively six times as follows: for each wash, DMF (2.5-3.75 mL) was added to the vessel and the resulting mixture was periodically agitated for 30 seconds before the solution was drained through the frit. To the reaction vessel was added the amino acid (0.2 M in DMF, 2.0-2.5 mL, 8-10 equiv), then HATU (0.4 M in DMF, 1.0-1.25 mL, 8-10 equiv), and finally NMM (0.8 M in DMF, 1.0-1.25 mL, 20 equiv). The mixture was periodically agitated for 30-120 minutes, then the reaction solution was drained through the frit. The resin was washed successively six times as follows: for each wash, DMF (2.5-3.0 mL) was added and the resulting mixture was periodically agitated for 30 seconds before the solution was drained through the frit. The resulting resin was used directly in the next step.

### Single-Coupling Manual Addition Procedure:

To the reaction vessel containing the resin from the previous step was added DMF (3.0-3.75 mL) three times, upon which the mixture was agitated for 30 seconds before the solvent was drained through the frit each time. To the resin was added piperidine:DMF (20:80 v/v, 3.0-3.75 mL). The mixture was periodically agitated for 5.0 minutes and then the solution was drained through the frit. To the reaction vessel was added piperidine:DMF (20:80 v/v, 3.0-3.75 mL). The mixture was periodically agitated for 5.0 minutes and then the solution was drained through the frit. The mixture was periodically agitated for 5.0 minutes and then the solution was drained through the frit. The resin was washed successively six times as follows: for each wash, DMF (3.0-3.75 mL) was added to the vessel and the resulting mixture was periodically agitated for 30 seconds before the solution was drained through the frit. To the reaction vessel was added the premixed amino acid (2.0-5.0 equiv) and HATU (0.4 M in DMF, 2.0-5.0 equiv), then NMM (0.8 M in DMF, 4.0-10.0 equiv) and the molar ratio for amino acid, HATU, and NMM is 1:1:2. The mixture was periodically agitated for 2-6 hours, then the reaction solution was drained through the frit. The resin was washed successively four times as follows: for each wash, DMF (3.75 mL) was added and the resulting mixture was periodically agitated for 30 seconds before the solution was drained through the frit. The resulting resin was used directly in the next step.

### Double-Coupling Procedure:

To the reaction vessel containing resin from the previous step was added DMF (2.5-3.75 mL) three times, upon which the mixture was agitated for 30 seconds before the solvent was drained through the frit each time. To the reaction vessel was added piperidine:DMF (20:80 v/v, 3.0-3.75 mL). The mixture was periodically agitated for 5 minutes and then the solution was drained through the frit. To the reaction vessel was added piperidine:DMF (20:80 v/v, 3.0-3.75 mL). The mixture was periodically agitated for 5 minutes and then the solution was drained through the frit. The resin was washed successively six times as follows: for each wash, DMF (3.0-3.75 mL) was added and the resulting mixture was periodically agitated for 30 seconds before the solution was drained through the frit. To the reaction vessel was added the amino acid (0.2 M in DMF, 2.0-2.5 mL, 8-10 equiv), then HATU (0.4 M in DMF, 1.0-1.25 mL, 10 equiv), and finally NMM (0.8 M in DMF, 1.0-1.25 mL, 16-20 equiv). The mixture was periodically agitated for 1 hour, then the reaction solution was drained through the frit. The resin was washed twice with DMF (3.0-3.75 mL) and the resulting mixture was periodically agitated for 30 seconds before the solution was drained through the frit each time. To the reaction vessel was added the amino acid (0.2 M in DMF, 2.0-2.5 mL, 8-10 equiv), then HATU (0.4 M in DMF, 1.0-1.25 mL, 8-10 equiv), and finally NMM (0.8 M in DMF, 1.0-1.25 mL, 16-20 eq). The mixture was periodically agitated for 1-2 hours, then the reaction solution was drained through the frit. The resin was successively washed six times as follows: for each wash, DMF (3.0-3.75 mL) was added and the resulting mixture was periodically agitated for 30 seconds before the solution was drained through the frit. The resulting resin was used directly in the next step.

Peptoid Installation (50 μmol) Procedure:

To the reaction vessel containing the resin from the previous step was added piperidine: DMF (20:80 v/v, 3.75 mL). The mixture was periodically agitated for 5 minutes and then the solution was drained through the frit. To the reaction vessel was added piperidine: DMF (20:80 v/v, 3.75 mL). The mixture was periodically agitated for 5 minutes and then the solution was drained through the frit. The resin was washed successively six times as follows: for each wash, DMF (3.75 mL) was added to the vessel and the resulting mixture was periodically agitated for 30 seconds before the solution was drained through the frit. To the reaction vessel was added the bromoacetic acid (0.4 M in DMF, 2.5 mL, 10 eq), then DIC (0.4 M in DMF, 2.5 mL, 10 eq). The mixture was periodically agitated for 60 mins, then the reaction solution was drained through the frit. The resin was washed successively two times as follows: for each wash, DMF (3.75 mL) was added to the vessel and the resulting mixture was periodically agitated for 30 seconds before the solution was drained through the frit. To the reaction vessel was added the amine (0.4 M in DMF, 2.5 mL, 10 eq), the mixture was periodically agitated for 60 mins, and then the reaction solution was drained through the frit. The resin was washed successively five times as follows: for each wash, DMF (3.75 mL) was added to the vessel and the resulting mixture was periodically agitated for 30 seconds before the solution was drained through the frit. The resulting resin was used directly in the next step.

### Chloroacetic Anhydride Coupling:

To the reaction vessel containing resin from the previous step was added DMF (3.0-3.75 mL) three times, upon which the mixture was agitated for 30 seconds before the solvent was drained through the frit each time. To the reaction vessel containing the resin from the previous step was added piperidine:DMF (20:80 v/v, 3.0-3.75 mL). The mixture was periodically agitated for 5 minutes and then the solution was drained through the frit. To the reaction vessel was added piperidine:DMF (20:80 v/v, 3.0-3.75 mL). The mixture was periodically agitated for 5 minutes and then the solution was drained through the frit. The resin was washed successively six times as follows: for each wash, DMF (3.0-3.75 mL) was added through the top of the vessel and the resulting mixture was periodically agitated for 30 seconds before the solution was drained through the frit. To the reaction vessel was added the chloroacetic anhydride solution (0.4 M in DMF, 3.0-3.75 mL, 30 equiv), then NMM (0.8 M in DMF, 2.5 mL, 40 equiv). The mixture was periodically agitated for 15 minutes, then the reaction solution was drained through the frit. The resin was washed once as follows: DMF (5.0-6.25 mL) was added through the top of the vessel and the resulting mixture was periodically agitated for 30 seconds before the solution was drained through the frit. To the reaction vessel was added the chloroacetic anhydride solution (0.4 M in DMF, 3.75 mL, 30 equiv), then NMM (0.8 M in DMF, 2.5 mL, 40 equiv). The mixture was periodically agitated for 15 minutes, then the reaction solution was drained through the frit. The resin was washed successively six times as follows: for each wash, DMF (2.5 mL) was added through the top of the vessel and the resulting mixture was periodically agitated for 30 seconds before the solution was drained through the frit. The resin was washed successively four times as follows: for each wash, DCM (2.5 mL) was added through the top of the vessel and the resulting mixture was periodically agitated for 30 seconds before the solution was drained through the frit. The resulting resin was dried using a nitrogen flow for 10 mins before being used directly in the next step.

### Symphony X Methods:

All manipulations were performed under automation on a Symphony X peptide synthesizer (Protein Technologies). Unless noted, all procedures were performed in a 45-mL polypropylene reaction vessel fitted with a bottom frit. The reaction vessel connects to the Symphony X peptide synthesizer through both the bottom and the top of the vessel. DMF and DCM can be added through the top of the vessel, which washes down the sides of the vessel equally. The remaining reagents are added through the bottom of the reaction vessel and pass up through the frit to contact the resin. All solutions are removed through the bottom of the reaction vessel. “Periodic agitation” describes a brief pulse of N₂ gas through the bottom frit; the pulse lasts approximately 5 seconds and occurs every 30 seconds. A “single shot” mode of addition describes the addition of all the solution contained in the single shot falcon tube that is usually any volume less than 5 mL. Amino acid solutions were generally not used beyond two weeks from preparation. HATU solution was used within 14 days of preparation.

Sieber amide resin=9-Fmoc-aminoxanthen-3-yloxy polystyrene resin, where “3-yloxy” describes the position and type of connectivity to the polystyrene resin. The resin used is polystyrene with a Sieber linker (Fmoc-protected at nitrogen); 100-200 mesh, 1% DVB, 0.71 mmol/g loading.

Rink=(2,4-dimethoxyphenyl)(4-alkoxyphenyl)methanamine, where “4-alkoxy” describes the position and type of connectivity to the polystyrene resin. The resin used is Merrifield polymer (polystyrene) with a Rink linker (Fmoc-protected at nitrogen); 100-200 mesh, 1% DVB, 0.56 mmol/g loading.

2-Chlorotrityl chloride resin (2-Chlorotriphenylmethyl chloride resin), 50-150 mesh, 1% DVB, 1.54 mmol/g loading. Fmoc-glycine-2-chlorotrityl chloride resin, 200-400 mesh, 1% DVB, 0.63 mmol/g loading.

PL-FMP resin: (4-Formyl-3-methoxyphenoxymethyl)polystyrene.

Common amino acids used are listed below with side-chain protecting groups indicated inside parenthesis:
Fmoc-Ala-OH; Fmoc-Arg(Pbf)-OH; Fmoc-Asn(Trt)-OH; Fmoc-Asp(tBu)-OH; Fmoc-Bip-OH; Fmoc-Cys(Trt)-OH; Fmoc-Dab(Boc)-OH; Fmoc-Dap(Boc)-OH; Fmoc-Gln(Trt)-OH; Fmoc-Gly-OH; Fmoc-His(Trt)-OH; Fmoc-Hyp(tBu)-OH; Fmoc-Ile-OH; Fmoc-Leu-OH; Fmoc-Lys(Boc)-OH; Fmoc-Nle-OH; Fmoc-Met-OH; Fmoc-[N-Me]Ala-OH; Fmoc-[N-Me]Nle-OH; Fmoc-Orn(Boc)-OH, Fmoc-Phe-OH; Fmoc-Pro-OH; Fmoc-Sar-OH; Fmoc-Ser(tBu)-OH; Fmoc-Thr(tBu)-OH; Fmoc-Trp(Boc)-OH; Fmoc-Tyr(tBu)-OH; Fmoc-Val-OH and their corresponding D-amino acids.

The procedures of “Symphony X Method” describe an experiment performed on a 0.050 mmol scale, where the scale is determined by the amount of Sieber or Rink or 2-chlorotrityl or PL-FMP bound to the resin. This scale corresponds to approximately 70 mg of the Sieber amide resin described above. All procedures can be scaled beyond or under 0.050 mmol scale by adjusting the described volumes by the multiple of the scale. Prior to amino acid coupling, all peptide synthesis sequences began with a resin-swelling procedure, described below as “Resin-swelling procedure”. Coupling of amino acids to a primary amine N-terminus used the “Single-coupling procedure” described below. Coupling of amino acids to a secondary amine N-terminus or to the N-terminus of Arg(Pbf)- and D-Arg(Pbf)- or D-Leu used the “Double-coupling procedure” or the “Single-Coupling 2-Hour Procedure” described below. Unless otherwise specified, the last step of automated synthesis is the acetyl group installation described as “Chloroacetyl Anhydride Installation”. All syntheses end with a final rinse and drying step described as “Standard final rinse and dry procedure”.

### Resin-Swelling Procedure:

To a 45-mL polypropylene solid-phase reaction vessel was added Sieber amide resin (70 mg, 0.050 mmol). The resin was washed (swelled) three times as follows: to the reaction vessel was added DMF (5.0 mL) through the top of the vessel “DMF top wash” upon which the mixture was periodically agitated for 3 minutes before the solvent was drained through the frit.

### Single-Coupling Procedure:

To the reaction vessel containing the resin from the previous step was added piperidine:DMF (20:80 v/v, 4.0 mL). The mixture was periodically agitated for 5 minutes and then the solution was drained through the frit. To the reaction vessel was added piperidine:DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. To the reaction vessel was added the amino acid (0.2 M in DMF, 2.0 mL, 8 equiv), then HATU (0.4 M in DMF, 1.0 mL, 8 equiv), and finally NMM (0.8 M in DMF, 1.0 mL, 16 equiv). The mixture was periodically agitated for 1-2 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Single-Coupling 4 Equivalent Procedure:

To the reaction vessel containing the resin from the previous step was added piperidine: DMF (20:80 v/v, 4.0 mL). The mixture was periodically agitated for 5 minutes and then the solution was drained through the frit. To the reaction vessel was added piperidine: DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. To the reaction vessel was added the amino acid (0.2 M in DMF, 1.0 mL, 4 equiv), then HATU (0.2 M in DMF, 1.0 mL, 4 equiv), and finally NMM (0.8 M in DMF, 1.0 mL, 16 equiv). The mixture was periodically agitated for 1-2 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Double-Coupling Procedure:

To the reaction vessel containing the resin from the previous step was added piperidine:DMF (20:80 v/v, 4.0 mL). The mixture was periodically agitated for 5 minutes and then the solution was drained through the frit. To the reaction vessel was added piperidine:DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. To the reaction vessel was added the amino acid (0.2 M in DMF, 2.0 mL, 8 equiv), then HATU (0.4 M in DMF, 1.0 mL, 8 equiv), and finally NMM (0.8 M in DMF, 1.0 mL, 16 equiv). The mixture was periodically agitated for 1 hour, then the reaction solution was drained through the frit. The resin was washed successively two times as follows: for each wash, DMF (5.0 mL) was added through the top of the vessel and the resulting mixture was periodically agitated for 30 seconds before the solution was drained through the frit. To the reaction vessel was added the amino acid (0.2 M in DMF, 2.0 mL, 8 equiv), then HATU (0.4 M in DMF, 1.0 mL, 8 equiv), and finally NMM (0.8 M in DMF, 1.0 mL, 16 equiv). The mixture was periodically agitated for 1-2 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Double-Coupling 4 Equivalent Procedure:

To the reaction vessel containing the resin from the previous step was added piperidine: DMF (20:80 v/v, 4.0 mL). The mixture was periodically agitated for 5 minutes and then the solution was drained through the frit. To the reaction vessel was added piperidine: DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. To the reaction vessel was added the amino acid (0.2 M in DMF, 1.0 mL, 4 equiv), then HATU (0.2 M in DMF, 1.0 mL, 4 equiv), and finally NMM (0.8 M in DMF, 1.0 mL, 16 equiv). The mixture was periodically agitated for 1 hour, then the reaction solution was drained through the frit. The resin was washed successively two times as follows: for each wash, DMF (5.0 mL) was added through the top of the vessel and the resulting mixture was periodically agitated for 30 seconds before the solution was drained through the frit. To the reaction vessel was added the amino acid (0.2 M in DMF, 1.0 mL, 4 equiv), then HATU (0.2 M in DMF, 1.0 mL, 4 equiv), and finally NMM (0.8 M in DMF, 1.0 mL, 16 equiv). The mixture was periodically agitated for 1-2 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Single-Coupling Manual Addition Procedure A:

To the reaction vessel containing the resin from the previous step was added piperidine:DMF (20:80 v/v, 4.0 mL). The mixture was periodically agitated for 5 minutes and then the solution was drained through the frit. To the reaction vessel was added piperidine:DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. The reaction was paused. The reaction vessel was opened and the unnatural amino acid (2-4 equiv) in DMF (1-1.5 mL) was added manually using a pipette from the top of the vessel while the bottom of the vessel remained attached to the instrument, then the vessel was closed. The automatic program was resumed and HATU (0.4 M in DMF, 1.0 mL, 8 equiv) and NMM (0.8 M in DMF, 1.0 mL, 16 equiv) were added sequentially. The mixture was periodically agitated for 2-3 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Single-Coupling Manual Addition Procedure B:

To the reaction vessel containing the resin from the previous step was added piperidine:DMF (20:80 v/v, 4.0 mL). The mixture was periodically agitated for 5 minutes and then the solution was drained through the frit. To the reaction vessel was added piperidine:DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. The reaction was paused. The reaction vessel was opened and the unnatural amino acid (2-4 equiv) in DMF (1-1.5 mL) was added manually using a pipette from the top of the vessel while the bottom of the vessel remained attached to the instrument, followed by the manual addition of HATU (2-4 equiv, same equiv as the unnatural amino acid), then the vessel was closed. The automatic program was resumed and NMM (0.8 M in DMF, 1.0 mL, 16 equiv) was added sequentially. The mixture was periodically agitated for 2-3 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Single-Coupling Manual Addition Procedure C:

To the reaction vessel containing the resin from the previous step was added piperidine: DMF (20:80 v/v, 4.0 mL). The mixture was periodically agitated for 5 minutes and then the solution was drained through the frit. To the reaction vessel was added piperidine: DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. The reaction was paused. The reaction vessel was opened and the unnatural amino acid (2-4 equiv) in DMF (1-1.5 mL) containing HATU (an equimolor amount relative to the unnatural amino acid), and NMM (4-8 equiv) was added manually using a pipette from the top of the vessel while the bottom of the vessel remained attached to the instrument. The automatic program was resumed and the mixture was periodically agitated for 2-3 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Single-Coupling Manual Addition Procedure D:

To the reaction vessel containing the resin from the previous step was added piperidine: DMF (20:80 v/v, 4.0 mL). The mixture was periodically agitated for 5 minutes and then the solution was drained through the frit. To the reaction vessel was added piperidine: DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. The reaction was paused. The reaction vessel was opened and the unnatural amino acid (2-4 equiv) in DMF (1-1.5 mL) containing DIC (an equimolor amount relative to the unnatural amino acid), and HOAt (an equimolor amount relative to the unnatural amino acid), was added manually using a pipette from the top of the vessel while the bottom of the vessel remained attached to the instrument. The automatic program was resumed and the mixture was periodically agitated for 2-3 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

Peptoid Installation (50 μmol) Procedure:

To the reaction vessel containing the resin from the previous step was added piperidine: DMF (20:80 v/v, 3.0 mL). The mixture was periodically agitated for 5 minutes and then the solution was drained through the frit. To the reaction vessel was added piperidine: DMF (20:80 v/v, 3.0 mL). The mixture was periodically agitated for 5 minutes and then the solution was drained through the frit. The resin was washed successively six times as follows: for each wash, DMF (3.0 mL) was added through the top of the vessel and the resulting mixture was periodically agitated for 30 seconds before the solution was drained through the frit. To the reaction vessel was added bromoacetic acid (0.4 M in DMF, 1.0 mL, 8 eq), then DIC (0.4 M in DMF, 1.0 mL, 8 eq). The mixture was periodically agitated for 1 hour, then the reaction solution was drained through the frit. The resin was washed successively two times as follows: for each wash, DMF (4.0 mL) was added through the top of the vessel and the resulting mixture was periodically agitated for 30 seconds before the solution was drained through the frit. To the reaction vessel was added the amine (0.4 M in DMF, 2.0 mL, 16 eq). The mixture was periodically agitated for 1 hour, then the reaction solution was drained through the frit. The resin was washed successively five times as follows: for each wash, DMF (3.0 mL) was added through the top of the vessel and the resulting mixture was periodically agitated for 30 seconds before the solution was drained through the frit. The resulting resin was used directly in the next step.

### Chloroacetic Anhydride Coupling:

To the reaction vessel containing the resin from the previous step was added piperidine:DMF (20:80 v/v, 3.0 mL). The mixture was periodically agitated for 3.5 or 5 minutes and then the solution was drained through the frit. To the reaction vessel was added piperidine:DMF (20:80 v/v, 3.0 mL). The mixture was periodically agitated for 5 minutes and then the solution was drained through the frit. The resin was washed successively six times as follows: for each wash, DMF (3.0 mL) was added through the top of the vessel and the resulting mixture was periodically agitated for 30 seconds before the solution was drained through the frit. To the reaction vessel was added the chloroacetic anhydride solution (0.4 M in DMF, 2.5 mL, 20 equiv), then N-methylmorpholine (0.8 M in DMF, 2.0 mL, 32 equiv). The mixture was periodically agitated for 15 minutes, then the reaction solution was drained through the frit. The resin was washed twice as follows: for each wash, DMF (3.0 mL) was added through the top of the vessel and the resulting mixture was periodically agitated for 1.0 minute before the solution was drained through the frit. To the reaction vessel was added the chloroacetic anhydride solution (0.4 M in DMF, 2.5 mL, 20 equiv), then N-methylmorpholine (0.8 M in DMF, 2.0 mL, 32 equiv). The mixture was periodically agitated for 15 minutes, then the reaction solution was drained through the frit. The resin was washed successively five times as follows: for each wash, DMF (3.0 mL) was added through the top of the vessel and the resulting mixture was periodically agitated for 1.0 minute before the solution was drained through the frit. The resulting resin was used directly in the next step.

### Final Rinse and Dry Procedure:

The resin from the previous step was washed successively six times as follows: for each wash, DCM (5.0 mL) was added through the top of the vessel and the resulting mixture was periodically agitated for 30 seconds before the solution was drained through the frit. The resin was then dried using a nitrogen flow for 10 minutes. The resulting resin was used directly in the next step.

### Sonata Method:

All procedures unless noted were performed under automation on a Sonata peptide synthesizer (Protein Technologies). All procedures unless noted were performed in a 200 ml-glass reaction vessel that connects to the Sonata peptide synthesizer through both the bottom and the top of the vessel. All reagents are added through the bottom of the vessel, and washes are performed with one single top wash followed by five bottom washes. All solutions are removed through the bottom of the vessel. “Periodic agitation” describes a brief pulse of N₂ gas through the bottom frit; the pulse lasts approximately 5 seconds and occurs every 30 seconds. Mechanical agitation is performed during additions and throughout washes and/or reaction cycles. Chloroacetic acid/DIC solutions in DMF were used within 0.25 h of preparation. Amino acid solutions were not used beyond three days from preparation. HATU solutions were used within five days of preparation. DMF=N, N-dimethylformamide; NMM=N-methylmorpholine.

The procedures of “Prelude Method” describe an experiment performed on a 2 mmol scale, where the scale is determined by the amount of Sieber or Rink or 2-chlorotrityl or PL-FMP resin. The procedures described below can be scaled up beyond 2 mmol scale simply by adjusting the described volumes by the multiple of the scale. The outline of a general synthesis is as follows: Prior to amino acid coupling, all peptide synthesis sequences began with a resin-swelling procedure, described below as “Resin-swelling procedure”. Coupling of amino acids to a primary amine N-terminus used the “Single-coupling procedure” described below. Coupling of amino acids to a secondary amine N-terminus or to the N-terminus of Arg(Pbf)- and D-Arg(Pbf)- used the “Double-coupling procedure” described below.
Swelling of resin (automated)
Amino acid coupling steps (repetition of general procedures A & B; automated)
Capping with chloroacetic acid (automated)
Global deprotection (manual)
Cyclization (manual)

### Resin-Swelling and First-Coupling Procedure:

Note: This procedure contains a swelling step and is used as the first coupling cycle.

To a 200-mL glass reaction vessel was added the resin. The resin was washed successively four times as follows: for each wash, DMF (9 seconds, ˜45 mL) was added through the top of the vessel and the resulting mixture was mechanically and periodically agitated for 1 minute before the solution was drained through the frit. To the reaction vessel containing resin from the previous step was added piperidine: DMF (20:80 v/v, 10 second delivery ˜50 mL). The mixture was mechanically and periodically agitated for 5 minutes and then the solution was drained through the frit. To the reaction vessel was added piperidine: DMF (20:80 v/v, 10 seconds ˜50 mL). The mixture was mechanically and periodically agitated for 5 minutes and then the solution was drained through the frit. The resin was washed successively six time as follows: for each wash, DMF (9 seconds, ˜45 mL) was added to the top of the vessel and the resulting mixture was mechanically and periodically agitated for 1 minute before the solution was drained through the frit. To the reaction vessel was added the amino acid (0.2 M in DMF, 20 mL, 2 equiv), then HATU (0.4 M in DMF, 10 mL, 2 equiv), and finally NMM (0.8 M in DMF, 10 mL, 4 equiv). The mixture was mechanically and periodically agitated for 30 minutes, then the reaction solution was drained through the frit. The resin was washed successively five times as follows: for each wash, DMF (9 seconds, ˜45 mL) was added through the top of the vessel and the resulting mixture was mechanically and periodically agitated for 1 minute before the solution was drained through the frit. To the reaction vessel was added over 9 seconds ˜45 mL of 10% (v/v) acetic anhydride and 10% (v/v) IPEA in DMF. The mixture was mechanically and periodically agitated for 10 minutes, then the solution was drained through the frit. The resin was washed successively five times as follows: for each wash, DMF (9 seconds, ˜45 mL) was added through the bottom of the vessel and the resulting mixture was mechanically and periodically agitated for 90 seconds before the solution was drained through the frit. The resulting resin was used directly in the next step.

Single-Coupling with Capping Procedure:

To the reaction vessel containing the resin from the previous step was added piperidine: DMF (20:80 v/v, ˜50 mL, 10 second delivery). The mixture was mechanically and periodically agitated for 5 minutes and then the solution was drained through the frit. To the reaction vessel was added piperidine: DMF (20:80 v/v, ˜50 mL, 10 second delivery). The mixture was mechanically and periodically agitated for 5 minutes and then the solution was drained through the frit. The resin was washed successively 6 times as follows: for each wash, DMF (9 seconds, ˜45 mL) was added through the top of the vessel and the resulting mixture was mechanically and periodically agitated for 1 minute before the solution was drained through the frit. To the reaction vessel was added the amino acid (0.2 M in DMF, 20 mL, 2 equiv), then HATU (0.4 M in DMF, 10 mL, 2 equiv), and finally NMM (0.8 M in DMF, 10 mL, 4 equiv). The mixture was mechanically and periodically agitated for 30 minutes, then the reaction solution was drained through the frit. The resin was washed successively five times as follows: for each wash, DMF (9 seconds, ˜45 mL) was added through the top of the vessel and the resulting mixture was mechanically and periodically agitated for 1 minute before the solution was drained through the frit. To the reaction vessel was added over 9 seconds −45 mL of 10% (v/v) acetic anhydride and 10% (v/v) DIEA in DMF. The mixture was mechanically and periodically agitated for 10 minutes, then the solution was drained through the frit. The resin was washed successively five times as follows: for each wash, DMF (9 seconds, ˜45 mL) was added through the bottom of the vessel and the resulting mixture was mechanically and periodically agitated for 90 seconds before the solution was drained through the frit. The resulting resin was used directly in the next step.

Double-Coupling with Capping Procedure:

Note: There are two exposures of the amino acid and coupling reagents (“double-coupling”). This procedure is typically used if the reacting terminal amine is secondary rather than primary.

To the reaction vessel containing resin from the previous step was added piperidine: DMF (20:80 v/v, 10 second delivery, ˜50 mL). The mixture was mechanically and periodically agitated for 5 minutes and then the solution was drained through the frit. To the reaction vessel was added piperidine: DMF (20:80 v/v, 10 seconds, ˜50 mL). The mixture was mechanically and periodically agitated for 5 minutes and then the solution was drained through the frit. The resin was washed successively six times as follows: for each wash, DMF (9 seconds, ˜45 mL) was added through the top of the vessel and the resulting mixture was mechanically and periodically agitated for 1 minute before the solution was drained through the frit. To the reaction vessel was added the amino acid (0.2 M in DMF, 20 mL, 2 equiv), then HATU (0.4 M in DMF, 10 mL, 2 equiv), and finally NMM (0.8 M in DMF, 10 mL, 4 equiv). The mixture was mechanically and periodically agitated for 30 minutes, then the reaction solution was drained through the frit. The resin was twice washed as follows: for each wash, DMF (9 seconds, ˜45 mL) was added through the top of the vessel and the resulting mixture was mechanically and periodically agitated for 1 minute before the solution was drained through the frit. To the reaction vessel was added the amino acid (0.2 M in DMF, 20 mL, 2 equiv), then HATU (0.4 M in DMF, 10 mL, 2 equiv), and finally NMM (0.8 M in DMF, 10 mL, 4 eq). The resin was washed successively five times as follows: for each wash, DMF (9 seconds, ˜45 mL) was added through the top of the vessel and the resulting mixture was mechanically and periodically agitated for 1 minute before the solution was drained through the frit. To the reaction vessel was added over 9 seconds ˜45 mL of 10% (v/v) acetic anhydride and 10% (v/v) IPEA in DMF. The mixture was mechanically and periodically agitated for 10 minutes, then the solution was drained through the frit. The resin was washed successively five times as follows: for each wash, DMF (9 seconds, ˜45 mL) was added through the bottom of the vessel and the resulting mixture was mechanically and periodically agitated for 90 seconds before the solution was drained through the frit. The resulting resin was used directly in the next step.

### Single Coupling, Extended Time Procedure:

Note: The coupling time is extended to two hours.

To the reaction vessel containing resin from the previous step was added piperidine: DMF (20:80 v/v, 10 second delivery, ˜50 mL). The mixture was mechanically and periodically agitated for 5 minutes and then the solution was drained through the frit. To the reaction vessel was added piperidine: DMF (20:80 v/v, 10 seconds, ˜50 mL). The mixture was mechanically and periodically agitated for 5 minutes and then the solution was drained through the frit. The resin was washed successively six times as follows: for each wash, DMF (9 seconds, ˜45 mL) was added through the bottom of the vessel and the resulting mixture was mechanically and periodically agitated for 1 minute before the solution was drained through the frit. To the reaction vessel was added the amino acid (0.2 M in DMF, 20 mL, 2 equiv), then HATU (0.4 M in DMF, 10 mL, 2 equiv), and finally NMM (0.8 M in DMF, 10 mL, 4 equiv). The mixture was mechanically and periodically agitated for 120 minutes, then the reaction solution was drained through the frit. The resin was washed successively five times as follows: for each wash, DMF (9 seconds, ˜45 mL) was added through the top of the vessel and the resulting mixture was mechanically and periodically agitated for 1 minute before the solution was drained through the frit. To the reaction vessel was added over 9 seconds ˜45 mL of 10% (v/v) acetic anhydride and 10% (v/v) IPEA in DMF. The mixture was mechanically and periodically agitated for 10 minutes, then the solution was drained through the frit. The resin was washed successively five times as follows: for each wash, DMF (9 seconds, ˜45 mL) was added through the bottom of the vessel and the resulting mixture was mechanically and periodically agitated for 90 seconds before the solution was drained through the frit. The resulting resin was used directly in the next step.

### Final Coupling Procedure:

Note: This condition differs from General Procedure B in the fact that it contains a final DCM wash of the peptide to prevent any loss of the terminal FMOC from the linear peptide

To the reaction vessel containing resin from the previous step was added piperidine: DMF (20:80 v/v, 10 second delivery, ˜50 mL). The mixture was mechanically and periodically agitated for 5 minutes and then the solution was drained through the frit. To the reaction vessel was added piperidine: DMF (20:80 v/v, 10 seconds, ˜50 mL). The mixture was mechanically and periodically agitated for 5 minutes and then the solution was drained through the frit. The resin was washed successively 1 time as follows: DMF (9 seconds, ˜45 mL) was added through the top of the vessel and the resulting mixture was mechanically and periodically agitated for 1 minute before the solution was drained through the frit. The resin was washed successively 5 times as follows: for each wash, DMF (9 seconds, ˜45 mL) was added through the top of the vessel and the resulting mixture was mechanically and periodically agitated for 1 minute before the solution was drained through the frit. To the reaction vessel was added the amino acid (0.2 M in DMF, 20 mL, 2 equiv), then HATU (0.4 M in DMF, 10 mL, 2 equiv), and finally NMM (0.8 M in DMF, 10 mL, 4 equiv). The mixture was mechanically and periodically agitated for 30 minutes, then the reaction solution was drained through the frit. The resin was twice washed as follows: for each wash, DMF (9 seconds, ˜45 mL) was added through the top of the vessel and the resulting mixture was mechanically and periodically agitated for 1 minute before the solution was drained through the frit. To the reaction vessel was added the amino acid (0.2 M in DMF, 20 mL, 2 equiv), then HATU (0.4 M in DMF, 10 mL, 2 equiv), and finally NMM (0.8 M in DMF, 10 mL, 4 equiv). The resin was washed successively five times as follows: for each wash, DMF (9 seconds, ˜45 mL) was added to top of the vessel and the resulting mixture was mechanically and periodically agitated for 1 minute before the solution was drained through the frit. To the reaction vessel was added over 9 seconds ˜45 mL of 10% (v/v) acetic anhydride and 10% (v/v) DIEA in DMF. The mixture was mechanically and periodically agitated for 10 minutes, then the solution was drained through the frit. The resin was washed successively five times as follows: for each wash, DMF (9 seconds, ˜45 mL) was added through the bottom of the vessel and the resulting mixture was mechanically and periodically agitated for 90 seconds before the solution was drained through the frit. The resin was washed successively seven times as follows: for each wash, DCM (10 seconds, ˜50 mL) was added through the bottom of the vessel and the resulting mixture was mechanically and periodically agitated for 90 seconds before the solution was drained through the frit. The resulting resin was used directly in the next step.

### Manual Mode Chloroacetic Acid Capping Procedure:

To the reaction vessel containing resin from the previous step was added piperidine: DMF (20:80 v/v, 10 second delivery, ˜50 mL). The mixture was mechanically and periodically agitated for 5 minutes and then the solution was drained through the frit. To the reaction vessel was added piperidine: DMF (20:80 v/v, 10 seconds, ˜50 mL). The mixture was mechanically and periodically agitated for 5 minutes and then the solution was drained through the frit. The resin was washed successively six times as follows: for each wash, DMF (9 seconds, ˜45 mL) was added through the bottom of the vessel and the resulting mixture was mechanically and periodically agitated for 1 minute before the solution was drained through the frit. To an Erlenmeyer flask were added 1.9 g (10 equiv) of chloroacetic acid in 40 mL of DMF and 4.7 mL (15 equiv) of DIC. This prepared solution was added to the 200-mL glass reactor which contains the resin, using a manual mode, 2 seconds, ˜10 mL DMF is added through the bottom of the frit and the resulting mixture was mechanically and periodically agitated for 2.5 days. The reaction time is likely to be much shorter and can be monitored using the Kaiser ninhydrin test. The resin was washed successively five times as follows: for each wash, DMF (9 seconds, ˜45 mL) was added through the bottom of the vessel and the resulting mixture was mechanically and periodically agitated for 1 minute before the solution was drained through the frit. The resin was washed successively seven times as follows: for each wash, DCM (10 seconds, ˜50 mL) was added through the bottom of the vessel and the resulting mixture was mechanically and periodically agitated for 1 minute before the solution was drained through the frit.

Alternatively, when 4 equivalents of amino acids were used, acetic anhydrade capping was not performed (no capping) in the above single-coupling and double-couplings steps

### Manual Handling and Manipulation for all the Following Procedures:

Manual handling and manipulation for all following procedures: a “deprotection solution” was prepared by combining in a 200 mL Erlenmeyer: 1% by weight of dithiothreitol (75 mg), 2.5% by volume of triisopropylsilane (1.875 mL), and trifluoroacetic acid (75 mL). The deprotection solution was cooled in an ice water bath to 5° C. prior to addition to the resin. 0.8 mmol ˜4 g of resin were placed in a 100 mL peptide synthesis vessel, the cold “deprotection solution” was added in one portion, the mixture was capped and shaken on a shaker for 1.5 hours. The filtrate was collected equally in 8×50 mL polypropylene Falcon tubes. 30 mL ether were added to each tube, capped and shaken to provide a white precipitate. The tubes were chilled in a refrigerator for 1 hour prior to centrifugation. Each tube was centrifuged (3 min, 2500 rpm) and the ethereal layers discarded. The precipitate was washed with ether (3×20 mL) and the centrifugation was repeated to provide the crude linear chloroacylated peptide.

7.5 mL of acetonitrile were added to each tube, and then they were diluted to 35 mL with aqueous 0.1 M NH₄HCO₃, shaking the resulting mixture to dissolve all solids. Caution: CO₂ evolves from quenching the excess TFA with ammonium bicarbonate. All solutions were transferred to a freeze drying flask and each Falcon tube was washed with 5 mL of a 1:1 mixture of acetonitrile and aqueous 0.1 M NH₄HCO₃. The resulting solution was carefully adjusted to pH 8 using 1.0 M NaOH. The solution was allowed to stand overnight (18 h). Reaction completion was confirmed by HPLC MS. Then the mixture was frozen in a dry ice/acetone bath and lyophilized to provide a white solid for purification.

### General Deprotection, Cyclization, N-Methylation, Click, Suzuki Procedures:

### Global Deprotection Method A:

Unless noted, all manipulations were performed manually. The procedure of “Global Deprotection Method” describes an experiment performed on a 0.050 mmol scale, where the scale is determined by the amount of Sieber or Rink or Wang or chlorotrityl resin or PL-FMP resin. The procedure can be scaled beyond 0.05 mmol scale by adjusting the described volumes by the multiple of the scale. In a 50-mL falcon tube was added the resin and 2.0-5.0 mL of the cleavage cocktail (TFA:TIS:DTT, v/v/w=95:5:1). The volume of the cleavage cocktail used for each individual linear peptide can be variable. Generally, higher number of protecting groups present in the sidechain of the peptide requires larger volume of the cleavage cocktail. The mixture was shaken at room temperature for 1-2 hours, usually about 1.5 hour. To the suspension was added 35-50 mL of cold diethyl ether. The mixture was vigorously mixed upon which a significant amount of a white solid precipitated. The mixture was centrifuged for 3-5 minutes, then the solution was decanted away from the solids and discarded. The solids were suspended in Et₂O (30-40 mL); then the mixture was centrifuged for 3-5 minutes; and the solution was decanted away from the solids and discarded. For a final time, the solids were suspended in Et₂O (30-40 mL); the mixture was centrifuged for 3-5 minutes; and the solution was decanted away from the solids and discarded to afford the crude peptide as a white to off-white solid together with the cleaved resin after drying under a flow of nitrogen and/or under house vacuum. The crude was used at the same day for the cyclization step.

### Global Deprotection Method B:

Unless noted, all manipulations were performed manually. The procedure of “Global Deprotection Method” describes an experiment performed on a 0.050 mmol scale, where the scale is determined by the amount of Sieber or Rink or Wang or chlorotrityl resin or PL-FMP resin. The procedure can be scaled beyond 0.05 mmol scale by adjusting the described volumes by the multiple of the scale. In a 30-mL Bio-Rad poly-prep chromatography column was added the resin and 2.0-5.0 mL of the cleavage cocktail (TFA:TIS:H₂O:DTT, v/v/w=94:3:3:1). The volume of the cleavage cocktail used for each individual linear peptide can be variable. Generally, higher number of protecting groups present in the sidechain of the peptide requires larger volume of the cleavage cocktail. The mixture was shaken at room temperature for 1-2 hours, usually about 1.5 hour. The acidic solution was drained into 40 mL of cold diethyl ether and the resin was washed twice with 0.5 mL of TFA solution. The mixture was centrifuged for 3-5 minutes, then the solution was decanted away from the solids and discarded. The solids were suspended in Et₂O (35 mL); then the mixture was centrifuged for 3-5 minutes; and the solution was decanted away from the solids and discarded. For a final time, the solids were suspended in Et₂O (35 mL); the mixture was centrifuged for 3-5 minutes; and the solution was decanted away from the solids and discarded to afford the crude peptide as a white to off-white solid after drying under a flow of nitrogen and/or under house vacuum. The crude was used at the same day for the cyclization step.

### Cyclization Method A:

Unless noted, all manipulations were performed manually. The procedure of “Cyclization Method A” describes an experiment performed on a 0.05 mmol scale, where the scale is determined by the amount of Sieber or Rink or chlorotrityl or Wang or PL-FMP resin that was used to generate the peptide. This scale is not based on a direct determination of the quantity of peptide used in the procedure. The procedure can be scaled beyond 0.05 mmol scale by adjusting the described volumes by the multiple of the scale. The crude peptide solids from the global deprotection were dissolved in DMF (30-45 mL) in the 50-mL centrifuge tube at room temperature, and to the solution was added DIEA (1.0-2.0 mL) and the pH value of the reaction mixture above was 8. The solution was then allowed to shake for several hours or overnight or over 2-3 days at room temperature. The reaction solution was concentrated to dryness on speedvac or genevac EZ-2 and the crude residue was then dissolved in DMF or DMF/DMSO (2 mL). After filtration, this solution was subjected to single compound reverse-phase HPLC purification to afford the desired cyclic peptide.

### Cyclization Method B:

Unless noted, all manipulations were performed manually. The procedure of “Cyclization Method B” describes an experiment performed on a 0.05 mmol scale, where the scale is determined by the amount of Sieber or Rink or chlorotrityl or Wang or PL-FMP resin that was used to generate the peptide. This scale is not based on a direct determination of the quantity of peptide used in the procedure. The procedure can be scaled beyond 0.05 mmol scale by adjusting the described volumes by the multiple of the scale. The crude peptide solids in the 50-mL centrifuge tube were dissolved in CH₃CN/0.1 M aqueous solution of ammonium bicarbonate (1:1,v/v, 30-45 mL). The solution was then allowed to shake for several hours at room temperature. The reaction solution was checked by pH paper and LCMS, and the pH can be adjusted to above 8 by adding 0.1 M aqueous ammonium bicarbonate (5-10 mL). After completion of the reaction based on the disappearance of the linear peptide on LCMS, the reaction was concentrated to dryness on speedvac or genevac EZ-2. The resulting residue was charged with CH₃CN:H₂O (2:3, v/v, 30 mL), and concentrated to dryness on speedvac or genevac EZ-2. This procedure was repeated (usually 2 times). The resulting crude solids were then dissolved in DMF or DMF/DMSO or CH₃CN/H₂O/formic acid. After filtration, the solution was subjected to single compound reverse-phase HPLC purification to afford the desired cyclic peptide.

N-Methylation on-Resin Method A.

To the resin (50 μmol) in a Bio-Rad tube was added CH₂Cl₂ (2 mL) and shaken for 5 min at RT. 2-Nitrobenzene-1-sulfonyl chloride (44.3 mg, 200 μmol, 4 equiv) was added followed by the addition of 2,4,6-trimethylpyridine (0.040 mL, 300 μmol, 6 equiv). The reaction was shaken at RT for 2 h. The solvent was drained and the resin was rinsed with CH₂Cl₂ (5 mL×3), DMF (5 mL×3) and then THF (5 mL×3). To the resin was added THF (1 mL). Triphenylphosphine (65.6 mg, 250 μmol, 5 equiv), methanol (0.020 mL, 500 μmol, 10 equiv) and Diethyl azodicarboxylate or DIAD (0.040 mL, 250 μmol, 5 equiv). The mixture was shaken at RT for 2-16 h. The reaction was repeated. Triphenylphosphine (65.6 mg, 250 μmol, 5 equiv), methanol (0.020 mL, 500 μmol, 10 equiv) and Diethyl azodicarboxylate or DIAD (0.040 mL, 250 μmol, 5 equiv) were added. The mixture was shaken at RT for 1-16 h. The solvent was drained, and the resin was washed with THF (5 mL×3) and CHCl₃ (5 mL×3). The resin was air dried and used directly in the next step. The resin was shaken in DMF (2 mL). 2-Mercaptoethanol (39.1 mg, 500 μmol) was added followed by DBU (0.038 mL, 250 μmol, 5 equiv). The reaction was shaken for 1.5 h. The solvent was drained. The resin was washed with DMF (4×). Air dried and used directly in the next step.

N-Methylation On-Resin Method B (Turner, R. A. Et al, Org. Lett., 15(19):5012-5015 (2013)).

All manipulations were performed manually unless noted. The procedure of “N-methylation on-resin Method A” describes an experiment performed on a 0.100 mmol scale, where the scale is determined by the amount of Sieber or Rink linker bound to the resin that was used to generate the peptide. This scale is not based on a direct determination of the quantity of peptide used in the procedure. The procedure can be scaled beyond 0.10 mmol scale by adjusting the described volumes by the multiple of the scale. The resin was transferred into a 25 mL fritted syringe. To the resin was added piperidine:DMF (20:80 v/v, 5.0 mL). The mixture was shaken for 3 min. and then the solution was drained through the frit. The resin was washed 3 times with DMF (4.0 mL). To the reaction vessel was added piperidine:DMF (20:80 v/v, 4.0 mL). The mixture was shaken for 3 min. and then the solution was drained through the frit. The resin was washed successively three times with DMF (4.0 mL) and three times with DCM (4.0 mL). The resin was suspended in DMF (2.0 mL) and ethyl trifluoroacetate (0.119 mL, 1.00 mmol), 1,8-diazabicyclo[5.4.0]undec-7-ene (0.181 mL, 1.20 mmol). The mixture was placed on a shaker for 60 min. The solution was drained through the frit. The resin was washed successively three times with DMF (4.0 mL) and three times with DCM (4.0 mL). The resin was washed three times with dry THF (2.0 mL) to remove any residual water. In an oven dried 4.0 mL vial was added THF (1.0 mL) and triphenylphosphine (131 mg, 0.500 mmol) on dry 4 Å molecular sieves (20 mg). The solution was transferred to the resin and diisopropyl azodicarboxylate (0.097 mL, 0.5 mmol) was added slowly. The resin was stirred for 15 min. The solution was drained through the frit and the resin was washed three times with dry THF (2.0 mL) to remove any residual water. In an oven dried 4.0 mL vial was added THF (1.0 mL), triphenylphosphine (131 mg, 0.50 mmol) on dry 4 A molecular sieves (20 mg). The solution was transferred to the resin and diisopropyl azodicarboxylate (0.097 mL, 0.5 mmol) was added slowly. The resin was stirred for 15 min. The solution was drained through the frit. The resin was washed successively three times with DMF (4.0 mL) and three times with DCM (4.0 mL). The resin was suspended in Ethanol (1.0 mL) and THF (1.0 mL), and sodium borohydride (37.8 mg, 1.000 mmol) was added. The mixture was stirred for 30 min. and drained. The resin was washed successively three times with DMF (4.0 mL) and three times with DCM (4.0 mL).

### N-Alkylation On-Resin Procedure Method A:

A solution of the alcohol corresponding to the alkylating group (0.046 g, 1.000 mmol), triphenylphosphine (0.131 g, 0.500 mmol), and DIAD (0.097 mL, 0.500 mmol) in 3 mL of THF was added to nosylated resin (0.186 g, 0.100 mmol), and the reaction mixture was stirred for 16 hours at room temperature. The resin was washed three times with THF (5 mL), and the above procedure was repeated 1-3 times. Reaction progress was monitored by TFA micro-cleavage of small resin samples treated with a solution of 50 μL of TIS in 1 mL of TFA for 1.5 hours.

### N-Alkylation On-Resin Procedure Method B:

The nosylated resin (0.100 mmol) was washed three times with N-methylpyrrolidone (NMP) (3 mL). A solution of NMP (3 mL), Alkyl Bromide (20 eq, 2.000 mmol) and DBU (20 eq, 0.301 mL, 2.000 mmol) was added to the resin, and the reaction mixture was stirred for 16 hours at room temperature. The resin was washed with NMP (3 mL) and the above procedure was repeated once more. Reaction progress was monitored by TFA micro-cleavage of small resin samples treated with a solution of 50 μL of TIS in 1 mL of TFA for 1.5 hours.

### N-Nosylate Formation Procedure:

A solution of collidine (10 eq.) in DCM (2 mL) was added to the resin, followed by a solution of Nos-Cl (8 eq.) in DCM (1 mL). The reaction mixture was stirred for 16 hours at room temperature. The resin was washed three times with DCM (4 mL) and three times with DMF (4 mL). The alternating DCM and DMF washes were repeated three times, followed by one final set of four DCM washes (4 mL).

### N-Nosylate Removal Procedure:

The resin (0.100 mmol) was swelled using three washes with DMF (3 mL) and three washes with NMP (3 mL). A solution of NMP (3 mL), DBU (0.075 mL, 0.500 mmol) and 2-mercaptoethanol (0.071 mL, 1.000 mmol) was added to the resin and the reaction mixture was stirred for 5 minutes at room temperature. After filtering and washing with NMP (3 mL), the resin was re-treated with a solution of NMP (3 mL), DBU (0.075 mL, 0.500 mmol) and 2-mercaptoethanol (0.071 mL, 1.000 mmol) for 5 minutes at room temperature. The resin was washed three times with NMP (3 mL), four times with DMF (4 mL) and four times with DCM (4 mL), and was placed back into a Symphony reaction vessel for completion of sequence assembly on the Symphony peptide synthesizer.

### General Procedure for Preloading Amines on the PL-FMP Resin:

PL-FMP resin (Novabiochem, 1.00 mmol/g substitution) was swollen with DMF (20 mL/mmol) at room temperature. The solvent was drained and 10 mL of DMF was added, followed by the addition of the amine (2.5 mmol) and acetic acid (0.3 mL) into the reaction vessel. After 10-min agitation, sodium triacetoxyhydroborate (2.5 mmol) was added. The reaction was allowed to agitate overnight. The resin was washed by DMF (1×), THF/H₂O/AcOH (6:3:1) (2×), DMF (2×), DCM (3×), and dried. The resulting PL-FMP resin preloaded with the amine can be checked by the following method: Took 100 mg of above resin and reacted with benzoyl chloride (5 equiv), and DIEA (10 equiv) in DCM (2 mL) at room temperature for 0.5 h. The resin was washed with DMF (2×), MeOH (1×), and DCM (3×). The sample was then cleaved with 40% TFA/DCM (1 h). The product was collected and analyzed by HPLC and MS. Collected sample was dried and got weight to calculate resin loading.

### General Procedure for Preloading Fmoc-Amino Acids on Cl-Trityl Resin:

To a glass reaction vessel equipped with a frit was added the 2-Chloro-chlorotrityl resin mesh 50-150, (1.54 meq/gram, 1.94 grams, 3.0 mmole) to be swollen in DCM (5 mL) for 5 minutes. A solution of the acid (3.00 mmol, 1.0 eq) in DCM (5 mL) was added to the resin followed by DIPEA (2.61 mL, 15.00 mmol, 5.0 eq). The reaction was shaken at room temperature for 60 minutes. Add in DIEA (0.5 mL) and methanol (3 mL), shaken for an additional 15 minutes. The reaction solution was filtered through the frit and the resin was rinsed with DCM (4×5 mL), DMF (4×5 mL), DCM (4×5 mL), diethyl ether (4×5 mL), and dried using a flow of nitrogen. The resin loading can be determined as follows:

A sample of resin (13.1 mg) was treated with 20% piperidine/DMF (v/v, 2.0 mL) for 10 minutes with shaking. 1 mL of this solution was transferred to a 25.0 mL volumetric flask and diluted with methanol to a total volume of 25.0 mL. A blank solution of 20% piperidine/DMF (v/v, 1.0 mL) was diluted up with methanol in a volumetric flask to 25.0 mL. The UV was set to 301 nm and zero with the blank solution followed by the reading of the solution, Absorbance=1.9411. (1.9411/20 mg)*6.94=0.6736. Loading of the resin was measured to be 0.6736 mmol/g.

### Click Reaction On-Resin Method A:

This procedure describes an experiment performed on a 0.050 mmol scale. It can be scaled beyond or under 0.050 mmol scale by adjusting the described volumes by the multiple of the scale. The alkyne containing resin (50 μmol each) was transferred into Bio-Rad tubes and swollen with DCM (2×5 mL×5 mins) and then DMF (2×5 mL×5 mins). In a 200-mL bottle was charged with 30 time of the following: ascorbic acid (vitamin C, 0.026 g, 0.150 mmol), bis(2,2,6,6-tetramethyl-3,5-heptanedionato)copper(II) (10.75 mg, 0.025 mmol), DMF (1.5 mL), 2,6-lutidine (0.058 mL, 0.50 mmol) and THF (1.5 mL), followed by DIEA (0.087 mL, 0.50 mmol) and the corresponding azide used in the examples (1.0-2.0 equiv.). The mixture was stirred until everything was in solution. The DMF in the above Bio-Rad tube was drained, and the above click solution (3 mL each) was added to each Bio-Rad tube. The tubes were shaken overnight on an orbital shaker. Solutions were drained through the frit. The resins were washed with DMF (3×2 mL) and DCM (3×2 mL).

### Click Reaction On-Resin Method B:

This procedure describes an experiment performed on a 0.050 mmol scale. It can be scaled beyond or under 0.050 mmol scale by adjusting the described volumes by the multiple of the scale. The alkyne containing resin (50 μmol each) was transferred into Bio-Rad tubes and swell with DCM (2×5 mL×5 mins) and then DMF (2 5 mL×5 mins). In a separate bottle, nitrogen was bubbled into 4.0 mL of DMSO for 15 mins. To the DMSO was added copper iodide (9.52 mg, 0.050 mmol, 1.0 eq) (sonicated), lutidine (58 μL, 0.500 mmol, 10.0 eq) and DIEA (87 μL, 0.050 mmol, 10.0 eq). The solution was purged with nitrogen again. DCM was drained through the frit. In a separate vial, ascorbic acid (8.8 mg, 0.050 mmol, 1.0 eq) was dissolved into water (600 μL). Nitrogen was bubbled through the solution for 10 mins. Coupling partners were distributed in the tubes (0.050 mmol to 0.10 mmol, 1.0 to 2.0 eq) followed by the DMSO copper and base solution and finally ascorbic acid aqueous solution. The solutions were topped with a blanket of nitrogen and capped. The tube was put onto the rotatory mixer for 16 hours. Solutions were drained through the frit. The resins were washed with DMF (3×2 mL) and DCM (3×2 mL).

### Suzuki Reaction On-Resin Procedure:

In a Bio Rad tube is placed 50 umoles of dried Rink resin of a N-terminus Fmoc-protected linear polypeptide containing a 4-bromo-phenylalanine side chain. The resin was swelled with DMF (2×5 mL). To this was added a DMF solution (2 mL) of p-tolylboronic acid (0.017 g, 0.125 mmol), potassium phosphate (0.2 mL, 0.400 mmol) followed by the catalyst [1,1′-bis(di-tert-butylphosphino)ferrocene]dichloropalladium(II) [PdCl₂(dtbpf)] (3.26 mg, 5.00 μmol). The tube was shaken at RT overnight. The solution was drained and the resin was washed with DMF (5×3 mL) followed by alternating DCM (2×3 mL), then DMF (2×3 mL), and then DCM (5×3 mL). A small sample of resin was micro-cleaved using 235 μL of TIS in 1 mL TFA at RT for 1 h. The rest of the resin was used in the next step of peptide coupling or chloroacetic acid capping of the N-terminus.

### Solution Phase Click Reaction Method A:

To a 20-mL scintillation vial was added 100-fold of sodium ascorbate (sodium (R)-2-((S)-1,2-dihydroxyethyl)-4-hydroxy-5-oxo-2,5-dihydrofuran-3-olate) and copper(II) sulfate pentahydrate (CuSO₄:sodium ascorbate mol ratio: 1:3 to 1:5). The reaction was diluted with water. This solution was shaken at RT for 1-10 min. The resulting yellowish slurry was added to the reaction.

To the vial containing the alkyne and the azide (1.0-2.0 equiv) was added an equimolar amount of the above copper solution (CuSO₄: 0.3-1.0 equiv of the alkyne). The mixture was shaken at RT for 1-3 h and the progress was monitored by LC/MS. Additional amounts of azide or copper solution can be added to drive the triazole formation if required. After completion, the mixture was diluted with CH₃CN:aq. NH₄CO₃ solution (v/v 1:1), filtered, and purified on the same day via reverse phase HPLC purification.

### Solution Phase Click Reaction Method B:

A stock solution of CuSO₄ and sodium ascorbate was prepared by diluting a dry 1:2 to 1:3 mol ratio of copper(II) sulfate pentahydrate and sodium ascorbate to a concentration of 0.1-0.3 M with respect to copper sulfate pentahydrate. To a solution of the peptide alkyne in DMF (0.05-0.1 M) was added the corresponding azide used in the examples (1.0-2.0 equiv) followed by the above freshly prepared aqueous copper solution (0.03-1.0 equiv). The mixture was stirred at room temperature and monitored by LCMS. Additional amounts of azide or copper solution can be added to drive the triazole formation if required. Upon full conversion, the mixture was diluted, filtered and purified on the same day by reverse phase HPLC.

### Fatty Acid Chain Coupling Procedure A:

To the elaborated peptide was added DMF (2.0 mL), the fatty activated ester (0.077 to 0.205 mmol, 1.5 to 4.0 eq) and DIEA (0.036 to 0.072 mL, 0.205 mmol, 4.0-8.0 eq). The reaction was allowed to shake for 1 h. The reaction mixture was neutralized with a few drops of acetic acid and submitted for purification.

### Fatty Acid Chain Coupling Procedure B:

To the elaborated peptide was added DMF (2.0 mL), the fatty activated ester (0.077 to 0.205 mmol, 1.5 to 4.0 eq) and DIEA (0.036 to 0.072 mL, 0.205 mmol, 4.0-8.0 eq). The reaction was allowed to shake for 1 h. The reaction mixture was concentrated to dryness using a Biotage V10 apparatus. To the crude pdt was added 2.0 mL of a solution of TFA/water (90:10, v:v) and the solution was allowed to shake for 20 minutes. The reaction was then concentrated to dryness and redissolved in 2.0 mL of DMF to be submitted for purification.

### Symphony Dde/ivDde Deprotection Procedure:

The reaction vessel containing the resin from the previous step was washed successively two times as follows: for each wash, DMF (2.5 mL) was added through the top of the vessel and the resulting mixture was periodically agitated for 30 seconds before the solution was drained through the frit. The resin was washed successively five times as follows: for each wash, a solution of hydrazine in DMF (2% v/v, 2.5 mL) was added through the top of the vessel and the resulting mixture was periodically agitated for 5 minutes before the solution was drained through the frit. The resin was washed successively six times as follows: for each wash, DMF (2.5 mL) was added through the top of the vessel and the resulting mixture was periodically agitated for 30 seconds before the solution was drained through the frit. The resulting resin was used directly in the next step.

### General Purification Procedures:

The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at certain percentage of B, then a linear increase from this percentage to a higher percentage of B over 20-30 minutes, then a 0-minute hold at 100% B; Flow Rate: 20-40 mL/min; Column Temperature: 25 C. Fraction collection was triggered by MS and UV signals. Fractions containing the desired product were combined and dried via centrifugal evaporation. If the material was not pure based on the orthogonal analytical data, it was further purified via preparative LC/MS with the following conditions: Column: XBridge C18, 200 mm×30 mm, 5-μm particles; Mobile Phase A: 5:95 acetonitrile: water with ammonium acetate; Mobile Phase B: 95:5 acetonitrile: water with ammonium acetate; Gradient: a 0-minute hold at certain percentage of B, then a linear increase from the starting percentage of B over 20-30 minutes, then a 0-minute hold at 100% B; Flow Rate: 20-40 mL/min; Column Temperature: 25 C. Fraction collection was triggered by MS signals. Fractions containing the desired product were combined and dried via centrifugal evaporation. The yield and the purity of the product were determined.

Alternatively, based on the initial analytical data, the crude material was purified via preparative LC/MS with the following conditions: Column: XBridge C18, 200 mm×30 mm, 5-μm particles; Mobile Phase A: 5:95 acetonitrile: water with ammonium acetate; Mobile Phase B: 95:5 acetonitrile: water with ammonium acetate; Gradient: a 0-minute hold at certain percentage of B, then a linear increase from the starting percentage of B over 20 minutes, then a 0-minute hold at 100% B; Flow Rate: 40 mL/min; Column Temperature: 25 C. Fraction collection was triggered by MS signals. Fractions containing the desired product were combined and dried via centrifugal evaporation. If the material is not pure based on the orthogonal analytical data, it was further purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at certain percentage of B, then a linear increase from this percentage to a higher percentage of B over 20 minutes, then a 0-minute hold at 100% B; Flow Rate: 20 mL/min; Column Temperature: 25 C. Fraction collection was triggered by MS and UV signals. Fractions containing the desired product were combined and dried via centrifugal evaporation. The yield and the purity of the product were determined.

### Unnatural Amino Acid Synthesis:

### Preparation of (S)-2-((((9H-fluoren-9-yl)methoxy)carbonyl)-3-(1-(2-(tert-butoxy)-2-oxoethyl)-1H-indol-3-yl)propanoic acid

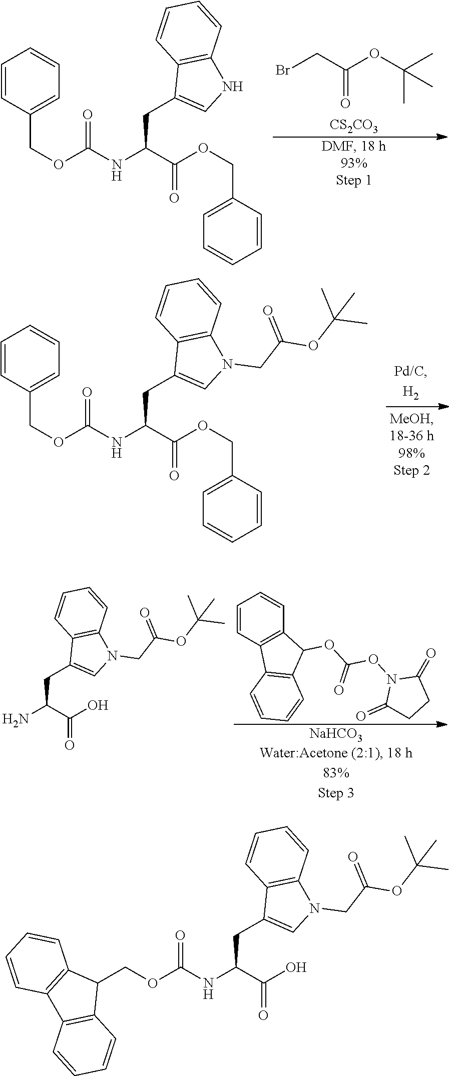

### Step 1

To a 0° C. solution of (S)-benzyl 2-(((benzyloxy)carbonyl)amino)-3-(1H-indol-3-yl) propanoate (25.0 g, 58.3 mmol) and cesium carbonate (20.9 g, 64.2 mmol) in DMF (200 mL) was added tert-butyl 2-bromoacetate (9.36 mL, 64.2 mmol). The solution was allowed to slowly warm up to RT with stirring for 18 h. The reaction mixture was poured into ice water:aq. 1N HCl (1:1) and then extracted with EtOAc. The organic layer was washed with brine, collected, dried over MgSO₄, filtered, and then concentrated in vacuo. The resulting solid was subjected to flash chromatography (330 g column, 0-50% EtOAc:Hex over 20 column volumes) to afford (S)-benzyl 2-(((benzyloxy)carbonyl)amino)-3-(1-(2-(tert-butoxy)-2-oxoethyl)-1H-indol-3-yl)propanoate as a white solid (29.6 g, 93%).

### Step 2

H₂ was slowly bubbled through a mixture of (S)-benzyl 2-(((benzyloxy)carbonyl)amino)-3-(1-(2-(tert-butoxy)-2-oxoethyl)-1H-indol-3-yl)propanoate (29.6 g, 54.5 mmol) and Pd—C (1.45 g, 1.36 mmol) in MeOH (200 mL) at RT for 10 min. The mixture was then stirred under positive pressure of H₂ while conversion was monitored by LCMS. After 48 h the reaction mixture was filtered through diatomaceous earth and evaporated to afford crude (S)-2-amino-3-(1-(2-(tert-butoxy)-2-oxoethyl)-1H-indol-3-yl)propanoic acid (17.0 g) which was carried into step three without additional purification.

### Step 3

To a solution of (S)-2-amino-3-(1-(2-(tert-butoxy)-2-oxoethyl)-1H-indol-3-yl)propanoic acid (5.17 g, 16.2 mmol) and sodium bicarbonate (6.8 g, 81 mmol) in acetone:water (50.0 mL:100 mL) was added (9H-fluoren-9-yl)methyl (2,5-dioxopyrrolidin-1-yl) carbonate (5.48 g, 16.2 mmol). The mixture stirred overnight upon which LCMS analysis indicated complete conversion. The vigorously stirred mixture was acidified via slow addition of aq 1N HCl. Once acidified, the mixture was diluted with DCM (150 mL), and the isolated organic phase was then washed with water, followed by brine. The organic layer was collected, dried over sodium sulfate, and concentrated under vacuum to afford the crude product. The crude material was purified via silica gel chromatography (330 g column, 20-80% EtOAc:Hex over 20 column 25 volumes) to afford (S)-2-((((9H-fluoren-9-yl)methoxy)carbonyl)amino)-3-(1-(2-(tertbutoxy)-2-oxoethyl)-1H-indol-3-yl)propanoic acid as a white foam (7.26 g, 83%). ¹H NMR (500 MHz, methanol-d₄) δ 7.80 (d, J=7.6 Hz, 2H), 7.67-7.60 (m, 2H), 7.39 (t, J=7.5 Hz, 2H), 7.32-7.22 (m, 3H), 7.18 (td, J=7.6, 0.9 Hz, 1H), 7.08 (td, J=7.5, 0.9 Hz, 1H), 7.04 (s, 1H), 4.54 (dd, J=8.4, 4.9 Hz, 1H), 4.36-4.23 (m, 2H), 4.23-4.14 (m, 1H), 30 3.43-3.35 (m, 2H), 3.25-3.09 (m, 1H), 1.55-1.38 (m, 9H). ESI-MS(+) m/z=541.3 (M+H).

### Preparation of (S)-2-((((9H-fluoren-9-yl)methoxy)carbonyl)amino)-3-(4-(2-(tert-butoxy)-2-oxoethoxy)phenyl)propanoic acid

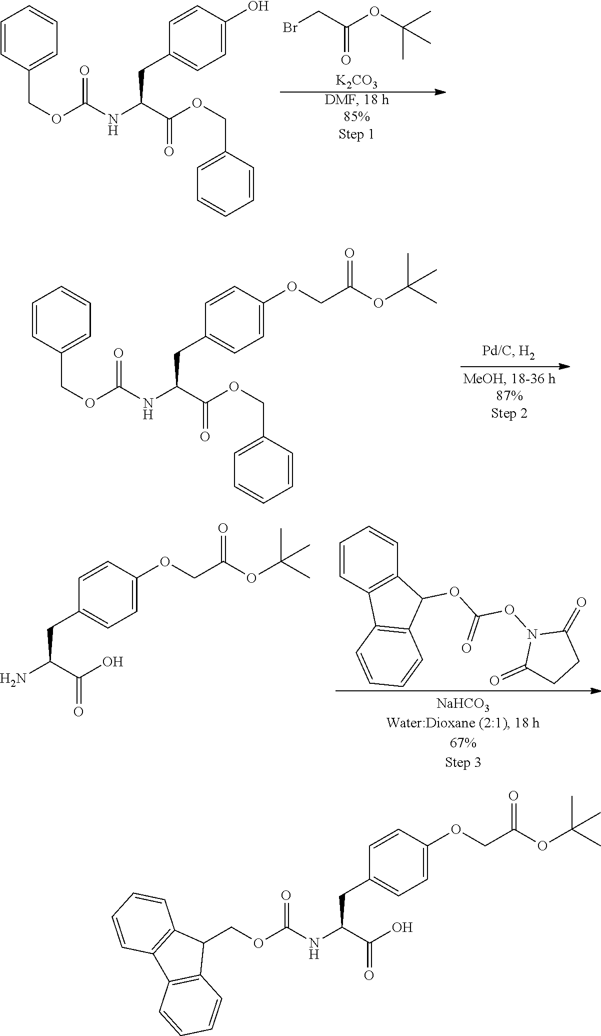

### Step 1

To a cooled stirred solution of (S)-benzyl 2-(((benzyloxy)carbonyl)amino)-3-(4-hydroxyphenyl)propanoate (70 g, 173 mmol) and K₂CO₃ (35.8 g, 259 mmol) in DMF (350 mL) was added tert-butyl-2-bromoacetate (30.6 mL, 207 mmol) dropwise and the resulting mixture was stirred at RT overnight. The reaction mixture was diluted with 10% brine solution (1000 mL) and extracted with ethyl acetate (2×250 mL). The combined organic layer was washed with water (500 mL), saturated brine solution (500 mL), dried over anhydrous sodium sulfate, filtered, and concentrated to afford colorless gum. The crude compound was purified by flash column chromatography using 20% ethyl acetate in petroleum ether as an eluent to afford a white solid (78 g, 85%).

### Step 2

The (S)-benzyl 2-(((benzyloxy)carbonyl)amino)-3-(4-(2-(tert-butoxy)-2-oxoethoxy)phenyl)propanoate (73 g, 140 mmol) was dissolved in MeOH (3000 mL) and purged with nitrogen for 5 min. To the above purged mixture was added Pd/C (18 g, 16.91 mmol) and stirred under hydrogen pressure of 3 kg for 15 hours. The reaction mixture was filtered through a bed of diatomaceous earth (Celite®) and washed with methanol (1000 mL). The filtrate was concentrated under vacuum to afford a white solid (36 g, 87%).

### Step 3

To a stirred solution of (S)-2-amino-3-(4-(2-(tert-butoxy)-2-oxoethoxy)phenyl)propanoic acid (38 g, 129 mmol) and sodium bicarbonate (43.2 g, 515 mmol) in water (440 mL) was added Fmoc-OSu (43.4 g, 129 mmol) dissolved in dioxane (440 mL) dropwise and the resulting mixture was stirred at RT overnight. The reaction mixture was diluted with 1.5 N HCl (200 mL) and water (500 mL) and extracted with ethyl acetate (2×250 mL). The combined organic layer was washed with water (250 mL), saturated brine solution (250 mL), and dried over Na₂SO₄, filtered, and concentrated to afford a pale yellow gum. The crude compound was purified by column chromatography using 6% MeOH in chloroform as an eluent to afford pale green gum. The gum was further triturated with petroleum ether to afford an off-white solid (45 g, 67%). ¹H NMR (400 MHz, DMSO-d₆) δ 12.86-12.58 (m, 1H), 7.88 (d, J=7.5 Hz, 2H), 7.73-7.61 (m, 3H), 7.58-7.47 (m, 1H), 7.44-7.27 (m, 4H), 7.18 (d, J=8.5 Hz, 2H), 6.79 (d, J=8.5 Hz, 2H), 4.57 (s, 2H), 4.25-4.10 (m, 4H), 3.34 (br s, 3H), 3.02 (dd, J=13.8, 4.3 Hz, 1H), 2.81 (dd, J=14.1, 10.5 Hz, 1H), 1.41 (s, 9H).

### Preparation of(S)-2-((((9H-fluoren-9-yl)methoxy)carbonyl)amino)-3-(4-(tert-butoxycarbonyl)phenyl)propanoic acid

### Step 1

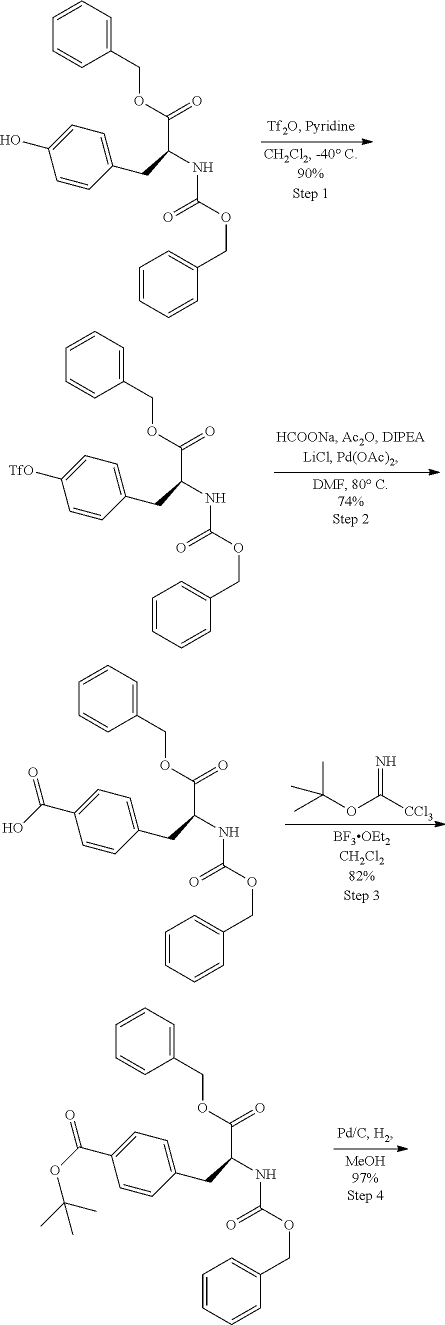

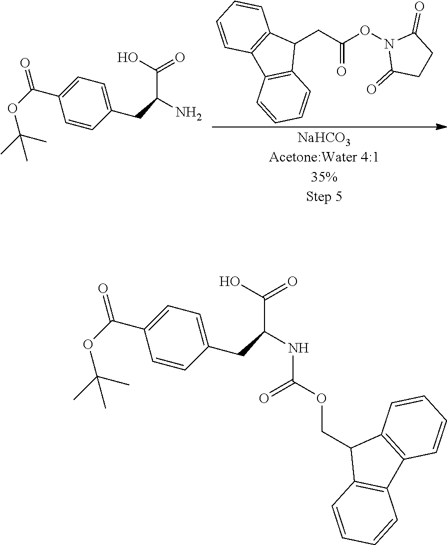 [SMILES: CC(C)(C)OC(=O)c1ccc(C[C@H](N)C(=O)O)cc1 ; O=C(CC1c2ccccc2-c2ccccc21)ON1C(=O)CCC1=O ; CC(C)(C)OC(=O)c1ccc(C[C@H](NC(=O)OCC2c3ccccc3-c3ccccc32)C(=O)O)cc1]

(S)-Benzyl 2-(((benzyloxy)carbonyl)amino)-3-(4-hydroxyphenyl)propanoate (10 g, 24.66 mmol) was taken in DCM (100 mL) in a 250 mL multi-neck round bottom flask under magnetic stirring with N₂ outlet. The reaction mixture was cooled to −40° C., pyridine (5.49 mL, 67.8 mmol) was added slowly and then stirred at the same temperature for 20 minutes, followed by addition of triflic anhydride (11.46 mL, 67.8 mmol) slowly at −40° C. and allowed to stir at −40° C. for 2 hours. The reaction mixture was quenched with water at −10° C., and then added citric acid solution (50 mL). The organic layer was extracted in DCM, and the separated organic layer was dried over anhydrous Na₂SO₄, filtered, and then evaporated to give (S)-benzyl 2-(((benzyloxy)carbonyl)amino)-3-(4-(((trifluoromethyl)sulfonyl)oxy)phenyl)propanoate (11.93 g, 22.20 mmol, 90% yield) as a pale yellow solid.

### Step 2

A solution of DMF (1500 mL) was purged with nitrogen for 10 min. To this was added sodium formate (114 g, 1676 mmol) and acetic anhydride (106 mL, 1123 mmol). Purging continued and the mixture was cooled to 0° C. DIPEA (194 mL, 1111 mmol) was added and the reaction mixture was allowed to stir for 1 h at RT under nitrogen atmosphere.

To a 10-liter autoclave was added DMF (3200 mL) and the system was purged with nitrogen. Under the nitrogen purging conditions, (S)-benzyl 2-(((benzyloxy)carbonyl)amino)-3-(4-(((trifluoromethyl)sulfonyl)oxy)phenyl)propanoate (300 g, 558 mmol), lithium chloride (71 g, 1675 mmol), 1,3-bis(diphenylphosphino)propane (24.17 g, 58.6 mmol) were added followed by the addition of palladium(II) acetate (12.9 g, 57.5 mmol). To this reaction mixture was added the above prepared solution and heated to 80° C. for 16 h.

The reaction mass was diluted with ethyl acetate and water. The phases were separated and the ethyl acetate layer was washed with water and brine solution, dried over anhydrous sodium sulphate, filtered, and concentrated. The crude material was added to a torrent column and was eluted with petroleum ether and ethyl acetate. The fractions at 30%-65% ethyl acetate in petroleum ether were concentrated to afford a cream solid (300 g), which was dissolved in ethyl acetate (700 mL) and petroleum ether was added slowly. At about 20% ethyl acetate in petroleum ether a white solid precipitated out, which was filtered and washed with 20% ethyl acetate in petroleum ether to obtain a white solid (180 g, yield 74%).

### Step 3

To a 2000-mL multi-neck round-bottomed flask was charged (S)-4-(3-(benzyloxy)-2-(((benzyloxy)carbonyl)amino)-3-oxopropyl)benzoic acid (130 g, 300 mmol), dichloromethane (260 mL) and cyclohexane (130 mL). To the slurry reaction mixture was added BF₃·OEt₂ (3.80 mL, 30.0 mmol) at room temperature, followed by the addition of tert-butyl 2,2,2-trichloroacetimidate (262 g, 1200 mmol) slowly at room temperature over 30 min. Upon addition, the slurry slowly started dissolving and at the end of the addition it was completely dissolved. The reaction mixture was allowed to stir at room temperature for 16 h. The reaction mixture was diluted with DCM and the remaining solids were removed by filtration. The filtrate was concentrated and purified by flash chromatography. The crude material was purified by Torrent using 1.5 Kg silicycle column. The product spot was eluted at 15% ethyl acetate/petroleum ether mixture. The collected fractions were concentrated to obtain a colorless liquid (120 g, yield 82%).

### Step 4

(S)-tert-Butyl 4-(3-(benzyloxy)-2-(((benzyloxy)carbonyl)amino)-3-oxopropyl)benzoate (200 g, 409 mmol) was dissolved in MeOH (4000 mL) and N₂ was purged for 10 min. Pd/C (27.4 g, 25.7 mmol) was added. The reaction was shaken under H₂ for 16 h at room temperature. The reaction mass was filtered through celite bed and the bed was washed with methanol. The obtained filtrate was concentrated to obtain a pale yellow solid. The obtained solid was stirred with 5% methanol:diethyl ether mixture for 15 min before being filtered, dried under vacuum to obtain a pale yellow solid. It was made slurry with 5% methanol in diethyl ether and stirred for 15 min, filtered, and dried to give (S)-2-amino-3-(4-(tert-butoxycarbonyl)phenyl)propanoic acid as a white solid (105 g, yield 97%). Analysis condition E: Retention time=0.971 min; ESI-MS(+) m/z [M+H]⁺: 266.2.

### Step 5

(S)-2-Amino-3-(4-(tert-butoxycarbonyl)phenyl)propanoic acid (122 g, 460 mmol) was dissolved in acetone (1000 mL) and then water (260 mL) and sodium bicarbonate (116 g, 1380 mmol) were added. It was cooled to 0° C. and Fmoc-OSu (155 g, 460 mmol) was added portionwise into the reaction mixture. After completion of addition it was stirred at room temperature for 16 h. The reaction mixture was diluted with dichloromethane (2 L) and then water was added (1.5 L). The organic layer was washed with saturated citric acid solution and extracted, and the aqueous layer was again extracted with DCM. The combined organic layer was washed with 10% citric acid solution, brine solution, and dried over Na₂SO₄, and evaporated to dryness. The obtained white solid was made slurry with diethyl ether, filtered, and dried to get the desired product as a white solid (80 g, yield 35%). ¹H NMR (400 MHz, DMSO-d₆) δ 7.87 (d, J=7.5 Hz, 2H), 7.83-7.73 (m, 3H), 7.60 (t, J=8.5 Hz, 2H), 7.51-7.24 (m, 7H), 4.26-4.11 (m, 4H), 3.45-3.27 (m, 4H), 3.17 (br dd, J=13.8, 4.3 Hz, 1H), 2.94 (dd, J=13.5, 11.0 Hz, 1H), 2.52-2.48 (m, 4H), 1.51 (s, 9H).

### Preparation of tert-butyl (R)-2-((((9H-fluoren-9-yl)methoxy)carbonyl)amino)-3-iodopropanoate

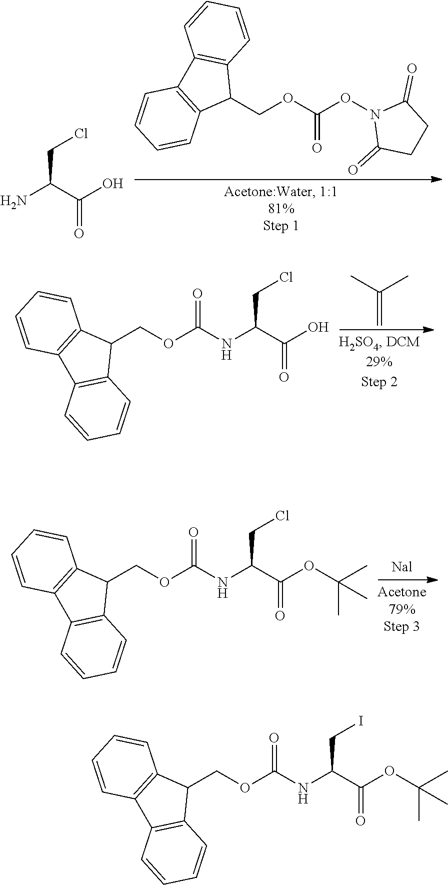

### Step 1

To a solution of (R)-2-amino-3-chloropropanoic acid hydrochloride (125 g, 781 mmol) in a 1:1 mixture of acetone (1 L) and water (1 L) was added Na₂CO₃ (182 g, 1719 mmol) followed by Fmoc-OSu (250 g, 742 mmol). The reaction was stirred at RT overnight. It was extracted with ethyl acetate (2×500 mL) and the aq. layer was acidified with 5N HCl. The HCl solution was extracted with ethyl acetate (1500 mL, then 2×500 mL). The combined organic layers were dried over anhydrous MgSO₄, filtered, and concentrated to give the crude product (R)-2-((((9H-fluoren-9-yl)methoxy)carbonyl)amino)-3-chloropropanoic acid. The product (220 g) was taken to the next step as such.

### Step 2

A solution of (R)-2-((((9H-fluoren-9-yl)methoxy)carbonyl)amino)-3-chloropropanoic acid (220 g, 636 mmol) in DCM (2 L) was cooled to −20° C. 2-Methylpropene (200 mL, 636 mmol) was bubbled into the solution for 15 mins, then H₂SO₄ (57.7 mL, 1082 mmol) was added and the mixture was stirred at RT overnight. To the reaction mixture was added water (500 mL). The layers were separated and the aqueous layer was extracted DCM (2×500 mL). The combined organic layers were dried over anhydrous MgSO₄, filtered, and evaporated. The crude was purified by flash chromatography using petroleum ether and ethyl acetate elution solvents. The desired fractions were combined and concentrated to give the product (R)-tert-butyl 2-((((9H-fluoren-9-yl)methoxy)carbonyl)amino)-3-chloropropanoate (83 g, 182 mmol, 29% yield).

### Step 3

To a solution of (R)-tert-butyl 2-((((9H-fluoren-9-yl)methoxy)carbonyl)amino)-3-chloropropanoate (80 g, 199 mmol) in acetone (1000 mL) was added sodium iodide (119 g, 796 mmol) and the reaction was heated to reflux for 40 hours. Acetone was removed by rotavap and the crude product was diluted with water (1000 mL) and DCM (1000 mL). The layers were separated and the organic layer was washed with aqueous saturated sodium sulphite solution (1000 mL) and brine (1000 mL). The organic layer was dried over anhydrous Na₂SO₄, filtered, and concentrated. The crude was purified by flash chromatography using 7 to 9% of ethyl acetate in petroleum ether. The desired product fractions were combined and concentrated to afford the product (R)-tert-butyl 2-((((9H-fluoren-9-yl)methoxy)carbonyl)amino)-3-iodopropanoate (83 g, 156 mmol, 79%). ¹H NMR (400 MHz, CDCl₃) δ 7.77 (d, J=7.5 Hz, 2H), 7.62 (d, J=7.5 Hz, 2H), 7.45-7.30 (m, 4H), 5.67 (br d, J=7.0 Hz, 1H), 4.54-4.32 (m, 3H), 4.30-4.21 (m, 1H), 3.71-3.50 (m, 2H), 1.56-1.48 (m, 9H).

### Preparation of (S)-2-((((9H-fluoren-9-yl)methoxy)carbonyl)amino)-3-(2-methyl-1H-indol-3-yl)propanoic acid

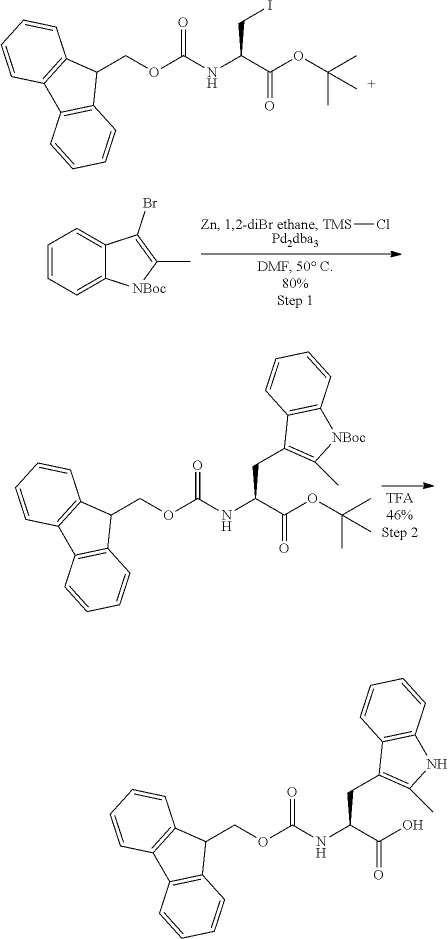

### Step 1

In a 100-mL three-neck, flame-dried, nitrogen-purged round-bottomed flask, zinc (2.319 g, 35.5 mmol) was added under argon atmosphere and the flask was heated to 150° C. using a hot gun and was purged with argon. To the reaction flask, DMF (50 mL) was added followed by the addition of 1,2-dibromoethane (0.017 mL, 0.20 mmol) and TMS-Cl (0.026 mL, 0.20 mmol) under argon atmosphere and then stirred for 10 min. To the reaction mixture (R)-tert-butyl 2-((((9H-fluoren-9-yl)methoxy)carbonyl)amino)-3-iodopropanoate (5 g, 10.14 mmol) was added and the reaction was stirred for 1 h. The reaction progress was monitored via TLC and LCMS, till the starting iodide was completely converted into the Zn-complex. The solution of organozinc reagent was allowed to cool to room temperature and then tris(dibenzylideneacetone)dipalladium(0) (Pd₂(dba)₃) (0.23 g, 0.25 mmol), dicyclohexyl(2′,6′-dimethoxy-[1,1′-biphenyl]-2-yl)phosphine (SPhos) (0.21 g, 0.51 mmol), and tert-butyl 3-bromo-2-methyl-1H-indole-1-carboxylate (3.77 g, 12.16 mmol) were added. The reaction mixture was allowed to stir at RT under a positive pressure of nitrogen for 1 h and then heated to 50° C. for 6 hrs. The reaction progress was monitored via LCMS. The mixture was diluted with EtOAc (700 mL) and filtered through diatomaceous earth (Celite®). The organic phase was washed with sat. NH₄Cl (250 mL), water (2×200 mL), and sat. NaCl (aq) (250 mL), dried over anhydrous Na₂SO₄(s), concentrated, and dried under vacuum to afford the crude compound (19 g). It was purified through ISCO flash chromatography using 330 g RediSep column and the product was eluted with 7 to 9% of ethyl acetate in petroleum ether. The above reaction and purification were repeated. The pure fractions were concentrated to give tert-butyl (S)-3-(2-((((9H-fluoren-9-yl)methoxy)carbonyl)amino)-3-(tert-butoxy)-3-oxopropyl)-2-methyl-1H-indole-1-carboxylate as a brownish solid (10.2 g. 95% pure, ca. 80% yield). Analysis condition G: Retention time=4.23 min; ESI-MS(+) m/z [M+2H][M-Boc-tBu+H]⁺: 441.2.

### Step 2

In a 25-mL multi neck, round-bottomed flask, DCM (65 mL) was added followed by (S)-tert-butyl 3-(2-((((9H-fluoren-9-yl)methoxy)carbonyl)amino)-3-(tert-butoxy)-3-oxopropyl)-2-methyl-1H-indole-1-carboxylate (6.5 g, 10.89 mmol) under nitrogen atmosphere at RT. The reaction mixture was cooled to 0° C., triethylsilane (4.18 mL, 26.1 mmol) was added followed by the addition of TFA (5.87 mL, 76 mmol) dropwise at 0° C. The temperature of the reaction mixture was slowly brought to RT and stirred at RT for 4 h. The reaction progress was monitored by TLC. To the reaction mixture, TFA (5.87 mL, 76 mmol) was added. The reaction mixture was stirred at RT overnight, and concentrated under reduced pressure. The crude material was triturated with hexanes and stored in cold room to give a brown colored solid (crude weight: 6.5 g). It was purified via reverse phase flash chromatography, and the pure fractions were concentrated to obtain the desired final product as an off-white powder (2.3 g, 46%). ¹H NMR (DMSO-d₆): δ ppm: 10.65 (s, 1H), 7.84 (d, J=9.12 Hz, 2H), 7.65 (d, J=9.12 Hz, 2H), 7.42-7.49 (m, 1H), 7.30-7.38 (m, 2H), 7.26-7.29 (m, 2H), 7.17-7.19 (m, 2H), 6.91-6.95 (m, 1H), 6.85-6.88 (t, J=7.85 Hz, 1H), 4-16-4.18 (m, 2H), 4.01-4.06 (m, 1H), 3.09-3.14 (m, 1H), 2.96-2.99 (m, 1H), 2.50 (s, 3H). Analysis condition F: Retention time=1.37 min; ESI-MS(+) m/z [M+2H][M+H]⁺: 441.2.

### Preparation of (S)-2-((((9H-fluoren-9-yl)methoxy)carbonyl)amino)-3-(7-methyl-1H-indol-3-yl)propanoic acid

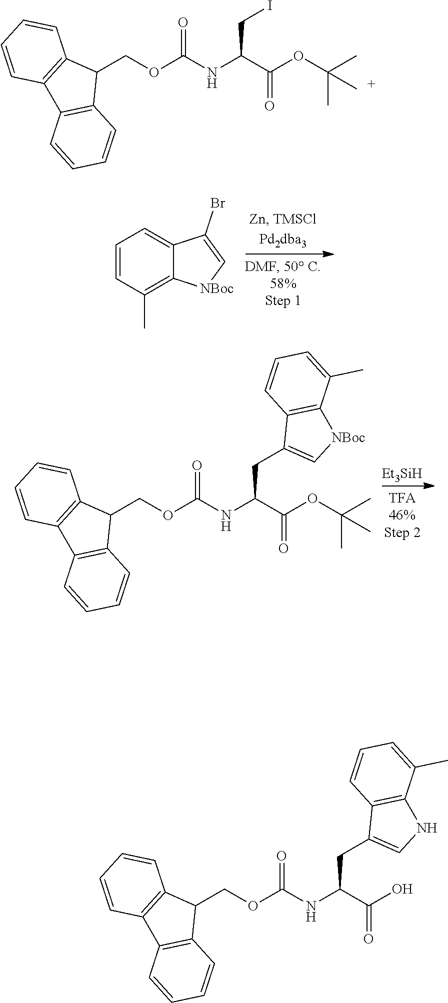

### Step 1

In a 50-mL round-bottomed flask, dry zinc (0.928 g, 14.19 mmol) was charged and flushed with argon three times and then the flask was heated to 150° C. for 5 min and then allowed to cool to room temperature and flushed with argon 3 times. DMF (20 mL) was added followed by the addition of 1,2-dibromoethane (6.99 μl, 0.081 mmol) and TMS-Cl (0.013 mL, 0.10 mmol). Successful zinc insertion was accompanied by a noticeable exotherm. After 5 min (R)-tert-butyl 2-((((9H-fluoren-9-yl)methoxy)carbonyl)amino)-3-iodopropanoate (2.0 g, 4.05 mmol) was added and the reaction was stirred for 30 min. In a 50-mL round-bottomed flask charged with Argon was added the above alkyl zinc reagent, tert-butyl 3-bromo-7-methyl-1H-indole-1-carboxylate (1.26 g, 4.05 mmol) followed by 2-dicyclohexylphosphino-2′,6′-dimethoxybiphenyl (SPhos) (0.083 g, 0.20 mmol) and Pd₂(dba)₃ (0.093 g, 0.101 mmol). After the addition, the reaction mixture was heated to 50° C. overnight. Another equivalents of Sphos and Pd₂(dba)₃ was added and heating continued for another 16 h. The reaction mixture was diluted with EtOAc (100 mL) and filtered through diatomaceous earth (Celite®). The organic phase was washed with sat. aq. NH₄Cl (100 mL), water (50 mL), and sat NaCl (100 mL), dried over anhydrous Na₂SO₄(s), concentrated, and dried under vacuum. After purification by flash chromatography the desired tert-butyl (S)-3-(2-((((9H-fluoren-9-yl)methoxy)carbonyl)amino)-3-(tert-butoxy)-3-oxopropyl)-2-methyl-1H-indole-1-carboxylate was obtained in 58% yield.

### Step 2

Final product was obtained following the same procedure of (S)-2-((((9H-fluoren-9-yl)methoxy)carbonyl)amino)-3-(2-methyl-1H-indol-3-yl)propanoic acid. TFA hydrolysis with triethylsilane afforded the desired (S)-2-((((9H-fluoren-9-yl)methoxy)carbonyl)amino)-3-(7-methyl-1H-indol-3-yl)propanoic acid as an off white solid in 64% yield after purification by reverse phase flash chromatography. Analysis condition E: Retention time=2.16 min; ESI-MS(+) m/z [M+H]⁺: 441.1. ¹H NMR (300 MHz, DMSO-d₆) Shift 12.70 (br s, 1H), 10.81 (br s, 1H), 7.88 (d, J=7.6 Hz, 2H), 7.76-7.56 (m, 2H), 7.49-7.21 (m, 5H), 7.17 (d, J=2.3 Hz, 1H), 6.94-6.84 (m, 2H), 4.29-4.13 (m, 3H), 4.07 (br s, 1H), 3.19 (br dd, J=14.7, 4.5 Hz, 1H), 3.01 (br dd, J=14.5, 9.6 Hz, 1H), 2.47-2.40 (m, 3H1), 0.02-−0.06 (m, 1H).

### Preparation of (S)-2-((((9H-fluoren-9-yl)methoxy)carbonyl)amino)-3-(quinolin-6-yl)propanoic acid

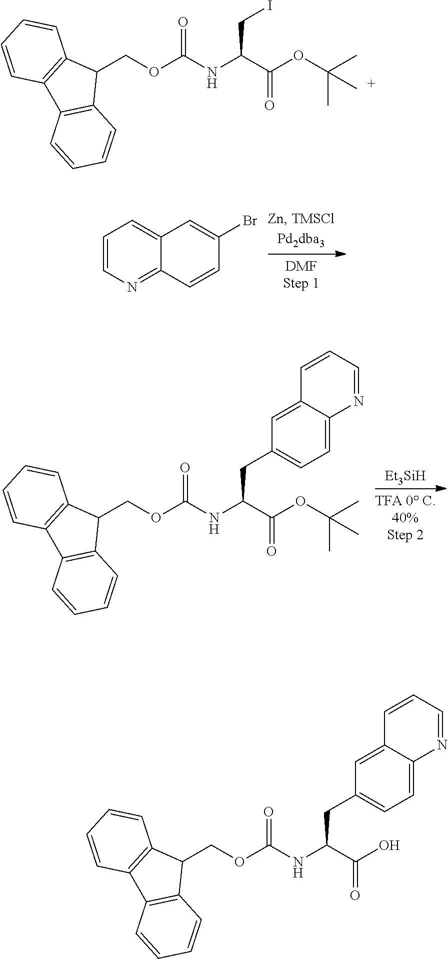

### Step 1

In a 25-mL round bottom flask, dry zinc (2.32 g, 35.5 mmol) was charged and argon was flushed three times. The flask was heated to 150° C. for 5 min and then allowed to cool to room temp and flushed with argon 3 times. DMF (50 mL) was added followed by the addition of 1,2-dibromoethane (0.017 mL, 0.20 mmol) and TMS-Cl (0.032 mL, 0.25 mmol). Successful zinc insertion was accompanied by a noticeable exotherm. After 5 min (R)-tert-butyl 2-((((9H-fluoren-9-yl)methoxy)carbonyl)amino)-3-iodopropanoate (5.0 g, 10.14 mmol) was added and the reaction was stirred for 30 min.

In a 250-mL round bottom flask purged with Argon was added DMF (50 mL), 6-bromoquinoline (2.53 g, 12.16 mmol), previously prepared solution of alkyl zinc reagent, (R)-tert-butyl 2-((((9H-fluoren-9-yl)methoxy)carbonyl)amino)-3-iodopropanoate (5.0 g, 10.14 mmol) followed by 2-dicyclohexylphosphino-2′,6′-diisopropoxy-1,1′-biphenyl (RuPhos) (0.24 g, 0.51 mmol) and Pd₂(dba)₃ (0.23 g, 0.25 mmol). The reaction mixture was allowed to stir at RT for 5 h and then heated to 50° C. for 16 h. It was cooled to RT and filtered over celite and rinsed with ethyl acetate. The solution was concentrated on rotovap. Purification by flash chromatography gave the desired compound as a thick brown liquid in quantitative yields. Analysis condition E: Retention time=3.47 min; ESI-MS(+) m/z [M+H]⁺: 495.2.

### Step 2

The final product was obtained following the same procedure of (S)-2-((((9H-fluoren-9-yl)methoxy)carbonyl)amino)-3-(2-methyl-1H-indol-3-yl)propanoic acid. TFA hydrolysis with triethylsilane afforded the desired (S)-2-((((9H-fluoren-9-yl)methoxy)carbonyl)amino)-3-(quinolin-6-yl)propanoic acid as a beige solid in 40% yield after solid-liquid extraction with diethyl ether and water. ¹H NMR (300 MHz, DMSO-d₆) δ 8.94 (br d, J=4.5 Hz, 1H), 8.49 (d, J=8.7 Hz, 1H), 8.01-7.92 (m, 2H), 7.85-7.79 (m, 3H), 7.65 (dd, J=8.3, 4.5 Hz, 1H), 7.55 (dd, J=7.2, 4.2 Hz, 2H), 7.36 (t, J=7.4 Hz, 2H), 7.26-7.14 (m, 2H), 4.32 (dd, J=10.6, 4.5 Hz, 1H), 4.18-4.08 (m, 3H), 3.38-3.29 (m, 2H), 3.11 (br d, J=10.6 Hz, 1H), 2.72 (s, 1H), 1.07 (t, J=7.0 Hz, 1H), −0.02 (s, 1H). Analysis condition E: Retention time=1.54 min; ESI-MS(+) m/z [M+H]⁺: 439.0.

### Preparation of (S)-2-((((9H-fluoren-9-yl)methoxy)carbonyl)amino)-3-(isoquinolin-6-yl)propanoic acid

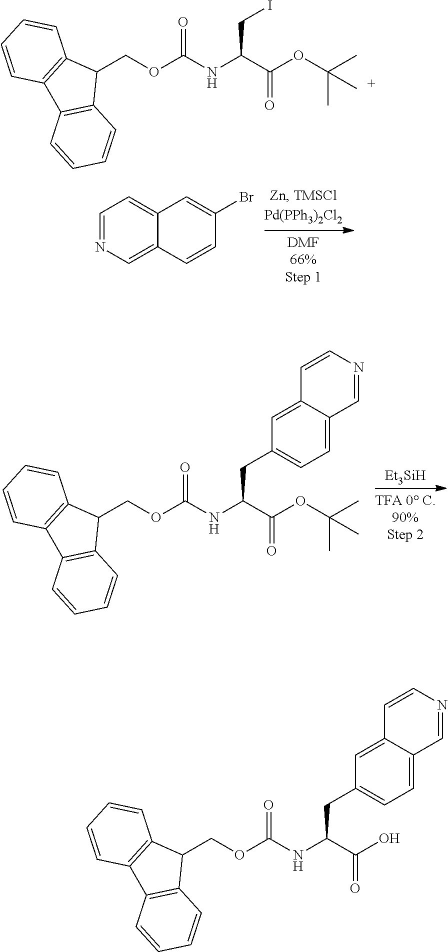

### Step 1

In a 50-mL three neck flame-dried round bottom flask zinc (1.392 g, 21.28 mmol) was added under argon atmosphere and the flask was heated to 150° C. using a hot gun and was purged with argon. To the reaction DMF (30 mL) was added followed by the addition of 1,2-dibromoethane (10.48 μl, 0.12 mmol) and TMS-Cl (0.016 mL, 0.12 mmol) under argon. The reaction was stirred for 10 minutes. To the reaction mixture (R)-tert-butyl 2-((((9H-fluoren-9-yl)methoxy)carbonyl)amino)-3-iodopropanoate (3.0 g, 6.08 mmol) was added and the reaction was stirred for 1 hr To the reaction mixture 6-bromoisoquinoline (1.52 g, 7.30 mmol) and bis-(triphenylphosphino)-palladous chloride (0.20 g, 0.30 mmol) were added and the reaction was stirred for 16 h. The reaction mixture was diluted with ethyl acetate (50 mL), filtered through celite and washed with ethyl acetate (50 mL). The filtrate was concentrated under reduced pressure to afford the crude product as a red thick gum. The crude was purified by flash chromatography using 40 to 42% EtOAc in petroleum ether. After concentration on rotovap tert-butyl (S)-2-((((9H-fluoren-9-yl)methoxy)carbonyl)amino)-3-(isoquinolin-6-yl)propanoate (2.0 g, 66%) was obtained as a yellow gum. Analysis condition B: Retention time=2.46 min; ESI-MS(+) m/z [M+H]⁺: 495.3.

### Step 2

The final product was obtained following the same procedure of (S)-2-((((9H-fluoren-9-yl)methoxy)carbonyl)amino)-3-(2-methyl-1H-indol-3-yl)propanoic acid. TFA hydrolysis with triethylsilane afforded the desired (S)-2-((((9H-fluoren-9-yl)methoxy)carbonyl)amino)-3-(isoquinolin-6-yl)propanoic acid as a grey solid in 90% yield after recrystallization in EtOAc and hexanes. ¹H NMR (400 MHz, METHANOL-d₄) δ 9.55 (s, 1H), 8.46 (d, J=6.5 Hz, 1H), 8.33 (d, J=8.5 Hz, 1H), 8.17 (d, J=6.0 Hz, 1H), 8.08 (s, 1H), 7.99-7.86 (m, 1H), 7.78 (dd, J=7.5, 4.0 Hz, 2H), 7.66-7.48 (m, 2H), 7.43-7.30 (m, 2H), 7.30-7.17 (m, 2H), 4.68 (dd, J=10.0, 4.5 Hz, 1H), 4.32-4.13 (m, 2H), 4.12-3.84 (m, 1H), 3.61 (dd, J=13.8, 4.8 Hz, 1H), 3.32-3.26 (m, 1H), 1.46 (s, 1H). Analysis condition B: Retention time=2.77 min; ESI-MS(+) m/z [M+H]⁺: 439.2.

### Preparation of (S)-2-((((9H-fluoren-9-yl)methoxy)carbonyl)amino)-3-(isoquinolin-4-yl)propanoic acid

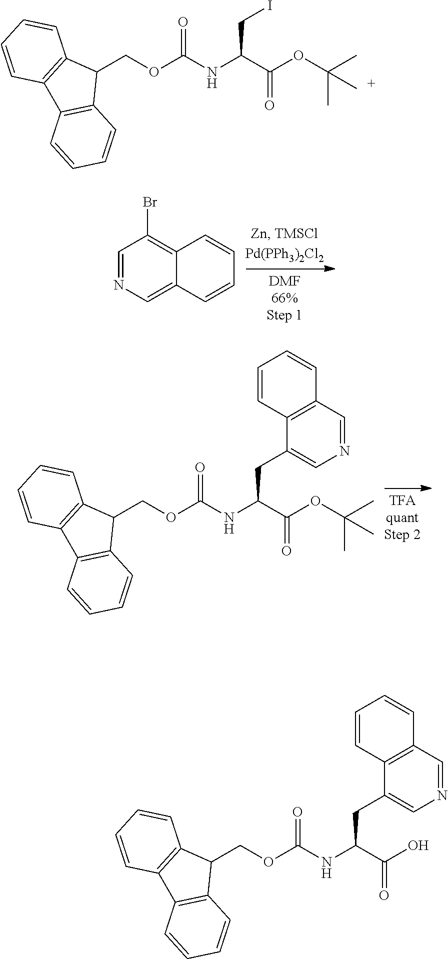

### Step 1

To a stirred mixture of zinc (2.319 g, 35.5 mmol) in DMF (50 mL) was added dibromomethane (0.071 mL, 1.014 mmol) and TMS-Cl (0.130 mL, 1.014 mmol). Exotherm was observed. The reaction mixture was stirred for 10 min. (R)-tert-butyl 2-((((9H-fluoren-9-yl)methoxy)carbonyl)amino)-3-iodopropanoate (5 g, 10.14 mmol) was added and again exotherm was observed. The reaction was allowed to stir for 1 h at room temperature. 2-Dicyclohexylphosphino-2′,6′-dimethoxybiphenyl (0.21 g, 0.51 mmol), tris(dibenzylideneacetone)dipalladium(0) (0.23 g, 0.25 mmol) and 4-bromoisoquinoline (2.11 g, 10.14 mmol) were added sequentially and the reaction was heated to 50° C. for 16 h. The reaction mixture was cooled to RT and treated with saturated ammonium chloride solution (200 mL). The crude was diluted with the ethyl acetate (300 mL). Layers were separated and the organic layer was washed with brine and dried over anhydrous sodium sulphate. After filtration and concentration the crude product was purified by flash chromatography eluting with 30% of ethyl acetate in petroleum ether to afford tert-butyl (S)-2-((((9H-fluoren-9-yl)methoxy)carbonyl)amino)-3-(isoquinolin-4-yl)propanoate (2.5 g, 50%). Analysis condition E: Retention time=3.44 min; ESI-MS(+) m/z [M+H]⁺: 495.2.

### Step 2

The final product was obtained following the same procedure of (S)-2-((((9H-fluoren-9-yl)methoxy)carbonyl)amino)-3-(2-methyl-1H-indol-3-yl)propanoic acid. TFA hydrolysis afforded the desired (S)-2-((((9H-fluoren-9-yl)methoxy)carbonyl)amino)-3-(isoquinolin-4-yl)propanoic acid as an off white solid in quantitative yield after purification diethyl ether trituration. ¹H NMR (400 MHz, DMSO-d₆) δ 9.55 (s, 1H), 8.52 (s, 1H), 8.44-8.24 (m, 2H), 8.18-8.00 (m, 1H), 7.95-7.80 (m, 4H), 7.59 (br d, J=7.5 Hz, 1H), 7.56 (br d, J=7.5 Hz, 1H), 7.47-7.34 (m, 2H), 7.34-7.24 (m, 2H), 4.46-4.30 (m, 1H), 4.25-4.02 (m, 3H), 3.69 (dd, J=14.1, 4.5 Hz, 1H), 3.37 (dd, J=14.1, 10.5 Hz, 1H), 0.10-0.11 (m, 1H). Analysis condition E: Retention time=1.57 min; ESI-MS(+) m/z [M+H]⁺: 441.2.

### Preparation of (S)-2-((((9H-fluoren-9-yl)methoxy)carbonyl)amino)-3-(4-(tert-butoxy)-3,5-difluorophenyl)propanoic acid

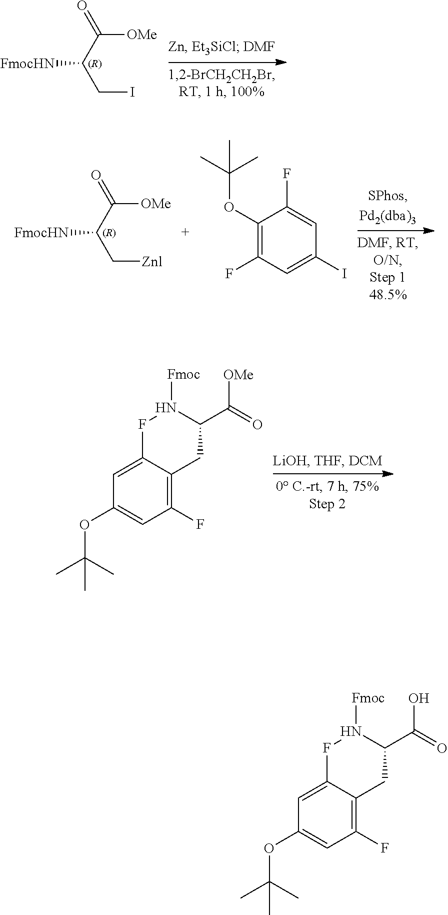

### Step 1

The compound was prepared following the same procedure of tert-butyl (S)-2-((((9H-fluoren-9-yl)methoxy)carbonyl)amino)-3-(isoquinolin-4-yl)propanoate. First Negishi coupling with methyl (R)-2-((((9H-fluoren-9-yl)methoxy)carbonyl)amino)-3-iodopropanoate at 50° C. afforded the desired methyl (S)-2-((((9H-fluoren-9-yl)methoxy)carbonyl)amino)-3-(4-(tert-butoxy)-2,6-difluorophenyl)propanoate (5.5 g, 48.5% yield) after purification by flash chromatography. Analysis condition E: Retention time=3.99 min; ESI-MS(+) m/z [M+NH₄]⁺: 527.2.

### Step 2

In a multi-neck round bottom flask methyl (S)-2-((((9H-fluoren-9-yl)methoxy)carbonyl)amino)-3-(4-(tert-butoxy)-3,5-difluorophenyl)propanoate (11 g, 21.59 mmol) was added followed by the addition of tetrahydrofuran (132 mL) under nitrogen atmosphere at RT. The reaction mixture was cooled to 0° C. and LiOH (1.09 g, 45.3 mmol) in water (132 mL) solution was added. The reaction was stirred for 3 h. It was concentrated under reduced pressure below 38° C. to remove the solvent. The crude compound was cooled to 0° C., sat. Citric acid solution was added to adjust the pH to 4-5. It was extracted with ethyl acetate (3×250 mL). The combined organic layer was washed with water (200 mL) followed by brine (200 mL). The organic layer dried over sodium sulphate, filtered and concentrated under reduced pressure to give the crude (12 g) as a colorless thick mass. The crude compound was purified through ISCO using 120 g RediSep column, the product was eluted with 20% of ethyl acetate in petroleum ether. The fractions were concentrated to give (S)-2-((((9H-fluoren-9-yl)methoxy)carbonyl)amino)-3-(4-(tert-butoxy)-3,5-difluorophenyl)propanoic acid (9.0 g, 82%, HPLC purity 97%) as a white fluffy solid. Analysis condition E: Retention time=3.62 min; ESI-MS(+) m/z [M+H]⁺: 513.2. ¹H NMR (CDCl₃, 400 MHz) d 7.75 (d, J=7.6 Hz, 2H), 7.60 (m, 2H), 7.39 (t, J=7.6 Hz, 2H), 7.30 (m, 2H), 6.71 (d, J=7.6 Hz, 2H), 5.26 (m, 1H), 4.65 (m, 1H), 4.48-4.38 (m, 2H), 4.20 (m, 1H), 3.14-2.99 (m, 1H), 1.35 (s, 9H).

### Preparation of (S)-2-((((9H-fluoren-9-yl)methoxy)carbonyl)amino)-3-(isoquinolin-8-yl)propanoic acid

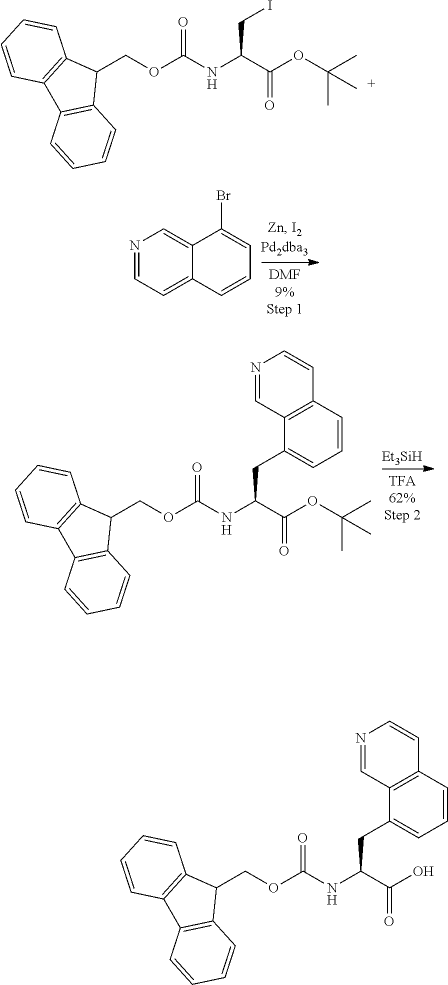

### Step 1

Zinc (0.79 g, 12.00 mmol) was added to a flame-dried, nitrogen-purged side arm round-bottomed flask. DMF (5 mL) was added via syringe, followed by a catalytic amount of iodine (0.16 g, 0.63 mmol). A color change of the DMF was observed from colorless to yellow and back again. Protected (R)-tert-butyl 2-((((9H-fluoren-9-yl)methoxy)carbonyl)amino)-3-iodopropanoate (1.97 g, 4.00 mmol) was added immediately, followed by a catalytic amount of iodine (0.16 g, 0.63 mmol). The solution was stirred at room temperature; successful zinc insertion was accompanied by a noticeable exotherm. The solution of organozinc reagent was allowed to cool to room temperature and then Pd₂(dba)₃ (0.088 g, 0.096 mmol), dicyclohexyl(2′,6′-dimethoxy-[1,1′-biphenyl]-2-yl)phosphine (0.082 g, 0.200 mmol) and 8-bromoisoquinoline (1.082 g, 5.20 mmol) were added sequentially. The reaction mixture was stirred at 50 C for 4 h. under a positive pressure of nitrogen. The reaction mixture was cooled to RT, diluted with EtOAc (200 mL) and passed through diatomaceous earth (Celite®). The organic solvent was washed with sat. aq. NH₄Cl (200 mL), water (150 mL), and sat. aq. NaCl (200 mL), dried over Na₂SO₄, concentrated, and dried under vacuum to afford the crude compound. It was purified using ISCO combiflash column chromatography (24 g silica gel column, hexanes/ethyl acetate as the eluents) to afford (S)-tert-butyl 2-((((9H-fluoren-9-yl)methoxy)carbonyl)amino)-3-(isoquinolin-8-yl)propanoate (380 mg, 0.768 mmol, 19.21% yield). Analysis condition G: Retention time=2.59 min; ESI-MS(+) m/z [M+H]⁺: 495.3.

### Step 2

(S)-tert-Butyl 2-((((9H-fluoren-9-yl)methoxy)carbonyl)amino)-3-(isoquinolin-8-yl)propanoate (380 mg. 0.768 mmol) was placed in 50-mL round bottom flask and was dissolved in DCM (8 mL). Triethylsilane (0.31 mL, 1.92 mmol) was added followed by trifluoroacetic acid (2.66 mL, 34.6 mmol). The reaction mixture was stirred at room temperature for 5 h. The solvents were evaporated, and the residue was dissolved in diethyl ether. The product was precipitated by the addition of petroleum ether. The resulting powder was then triturated with petroleum ether to yield (S)-2-((((9H-fluoren-9-yl)methoxy)carbonyl)amino)-3-(isoquinolin-8-yl)propanoic acid (320 mg, 0.712 mmol, 93% yield) as an off white solid. ¹H-NMR: (400 MHz, DMSO-d₆) δ ppm: 12.98 (bs, 1H), 9.79 (s, 1H), 8.62 (d, J=9.42 Hz, 1H), 8.22 (d, J=9.42 Hz, 1H), 8.06 (d, J=9.42 Hz, 1H), 7.84-7.93 (m, 4H), 7.74-7.76 (m, 1H), 7.56-7.58 (m, 1H), 7.38-7.42 (m, 2H), (m, 3H), 7.26-7.30 (m, 2H), 4.41 (m, 1H), 4.10-4.15 (m, 3H), 3.731-3.66 (m, 1H), 3.47-3.50 (m, 1H). Analysis condition G: Retention time=2.012 min; ESI-MS(+) m/z [M+H]⁺: 439.2 with 97.5% purity.

### Preparation of (S)-2-((((9H-fluoren-9-yl)methoxy)carbonyl)amino)-3-(7-fluoro-1H-indol-3-yl)propanoic acid

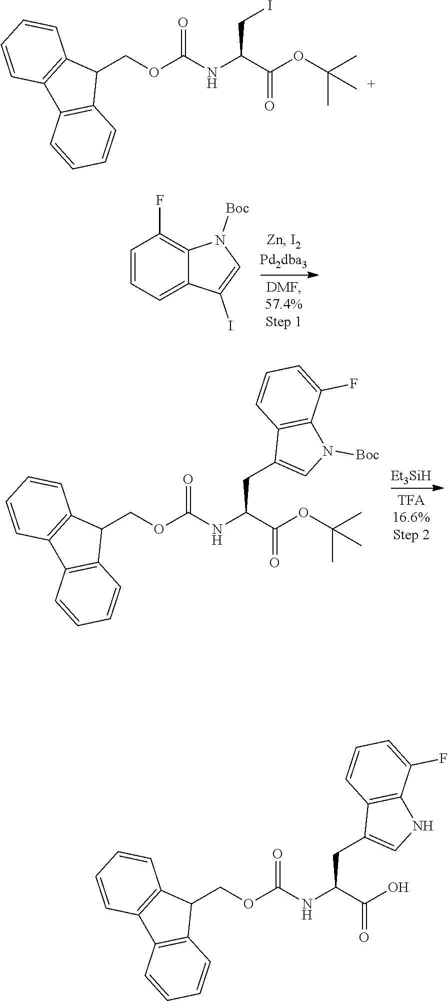

Synthesis of tert-butyl 6-fluoro-3-iodo-1H-indole-1-carboxylate from 6-fluoro-1H-indole: A solution of iodine (3.76 g, 14.80 mmol) in DMF (15 mL) was dropped to the solution of 6-fluoro-1H-indole (2 g, 14.80 mmol) and potassium hydroxide (2.076 g, 37.0 mmol) in DMF (15 mL) at room temperature and the mixture was stirred for 45 min. The reaction mixture was then poured on 200 mL of ice water containing 0.5% ammonia and 0.1% sodium disulfite. The mixture was placed in a refrigerator to ensure the complete precipitation. The precipitate was filtered, washed with 100 mL ice water and dried in vacuo to obtain 3.80 g. The solid was suspended in dichloromethane (25 mL). 4-Dimethylaminopyridine (160 mg, 10 mol %) and di-tert-butyl dicarbonate (4.84 g, 22.20 mmol) were dissolved in dichloromethane (15 mL), and were added to the reaction. The resulting mixture was stirred for 30 min at room temperature, washed with 0.1 N HCl (25 mL) and the aqueous phase was extracted with dichloromethane (3×35 mL, monitored by TLC). The combined organic layers were dried with sodium sulfate, the solvents were removed under reduced pressure to obtain tert-butyl 6-fluoro-3-iodo-1H-indole-1-carboxylate (4.16 g, 11.52 mmol, 78% yield) as an orange solid. ¹H-NMR (CDCl₃) δ ppm: 7.82 (d, J=8.23 Hz, 1H), 7.68 (s 1H), 7.30-7.34 (m, 1H), 7.03-7.08 (m, 1H), 1.66 (s, 9H).

### Step 2

Compound was prepared following the same procedure of (S)-tert-butyl 2-((((9H-fluoren-9-yl)methoxy)carbonyl)amino)-3-(isoquinolin-8-yl)propanoate. First Negishi coupling at 50° C. afforded the desired tert-butyl (S)-3-(2-((((9H-fluoren-9-yl)methoxy)carbonyl)amino)-3-(tert-butoxy)-3-oxopropyl)-7-fluoro-1H-indole-1-carboxylate (690 mg, 1.149 mmol, 57.4% yield) after purification by flash chromatography. Analysis condition H: Retention time=3.885 min; ESI-MS(+) m/z [M-Boc-tBu+H]⁺: 445.2

### Step 3

Final product was obtained following the same procedure of (S)-2-((((9H-fluoren-9-yl)methoxy)carbonyl)amino)-3-(isoquinolin-8-yl)propanoic acid TFA hydrolysis afforded the desired (S)-2-((((9H-fluoren-9-yl)methoxy)carbonyl)amino)-3-(7-fluoro-1H-indol-3-yl)propanoic acid as an off white powder (96 mg, 0.191 mmol, 16.63% yield) after purification by reverse phase prep HPLC (Column: 80 g size, Silisep C18, 19X150 mm, 5 μm, Mobile phases: A=10 mM ammonium acetate in water, B=MeoH. 15 mL/min flow Gradient: 0-20 min, 5-30% B, 20-55 min, 30-80% B, 55-60 min, 80-100% B, held at 100% B for 5 min. Compound was eluted at 75% B) followed by lyophilization. Analysis condition F: Retention time=1.367 min; ESI-MS(+) m/z [M+H]⁺: 445.3. ¹H-NMR (400 MHz, DMSO-d₆) δ ppm: 11.22 (s, 1H), 7.86 (d, J=8.72 Hz, 2H), 7.62-7.65 (m, 1H), 7.52-7.55 (m, 3H), 7.40-7.42 (m, 2H), 7.26-7.38 (m, 2H), 6.78-6.83 (m, 2H), 4.12-4.21 (m, 4H), 3.15-3.18 (m, 1H), 2.97-3.03 (m, 1H).

### Preparation of (2S,3S)-2-((((9H-fluoren-9-yl)methoxy)carbonyl)amino)-3-(1-(tert-butoxycarbonyl)-1H-indol-3-yl)butanoic acid

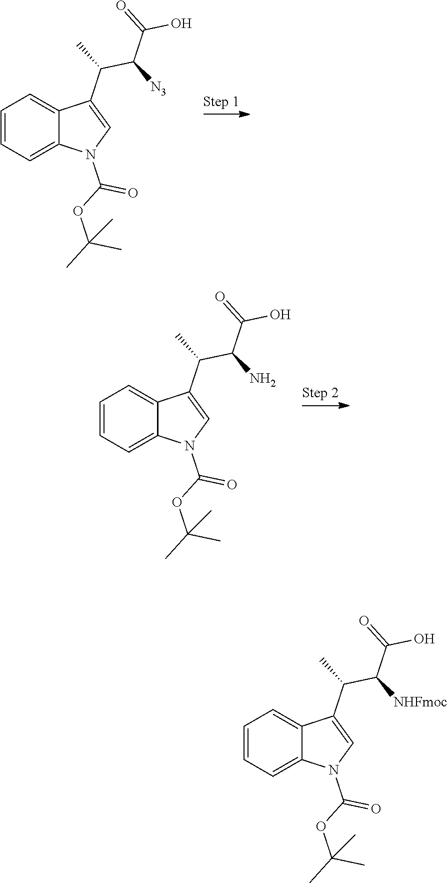

Compound (2S,3S)-2-azido-3-(I-(tert-butoxycarbonyl)-1H-indol-3-yl)butanoic acid was prepared following the procedure reported in Tetrahedron Letters 2001, 42, 4601-4603. The azide reduction step used different conditions as detailed below.

### Step 1

To a solution of (2S,3S)-2-azido-3-(1-(tert-butoxycarbonyl)-1H-indol-3-yl)butanoic acid (1000 mg, 2.90 mmol) in THF (58 mL) was added platinum(IV) oxide (132 mg, 0.58 mmol). The reaction mixture was evacuated and filled with hydrogen. The reaction mixture was allowed to stir at room temperature with a hydrogen balloon for 2 h. The reaction mixture was evacuated and back filled with nitrogen three times. The solution was filtered through diatomaceous earth (Celite®). The solvent was removed under vacuum and the crude residue was redissolved in EtOH. This solution was filtered through diatomaceous earth (Celite®) to give a clear solution which was concentrated under vacuum (0.89 g 96% yield). ¹H NMR (400 MHz, METHANOL-d₄) δ 8.13 (br d, J=8.0 Hz, 1H), 7.75 (d, J=7.8 Hz, 1H), 7.61 (s, 1H), 7.46-7.18 (m, 2H), 4.89 (s, 2H), 3.80 (d, J=6.5 Hz, 1H), 3.58 (t, J=7.2 Hz, 1H), 1.68 (s, 9H), 1.53 (d, J=7.3 Hz, 3H). Analysis condition B: Retention time=0.93 min; ESI-MS(+) m/z [M+H]⁺: 319.1.

### Step 2

To a solution of (2S,3S)-2-amino-3-(1-(tert-butoxycarbonyl)-1H-indol-3-yl)butanoic acid (3.96 g, 12.44 mmol) in MeOH (25 mL) was added (9H-fluoren-9-yl)methyl 2,5-dioxopyrrolidine-1-carboxylate (888 mg, 2.76 mmol) followed by Et₃N (0.385 mL, 2.76 mmol). The reaction was stirred for 2 h at room temperature. The solvent was removed under vacuum and the residue was redissolved in EtOAc and washed with 1 N HCl aqueous solution then brine. The organic layer was collected, dried over anhydrous sodium sulfate, and concentrated under vacuum to give the desired product (1.3 g, 89% yield) which was not purified further. ¹H NMR (500 MHz, DMSO-d₆) δ 12.78 (br s, 1H), 8.07-7.80 (m, 2H), 7.76-7.48 (m, 4H), 7.46-7.15 (m, 6H), 5.75 (s, 1H), 4.44 (t, J=8.2 Hz, 1H), 4.33-4.22 (m, 1H), 4.19-4.07 (m, 2H), 1.56 (s, 9H), 1.39-1.27 (m, 3H).

### Preparation of (S)-2-((((9H-fluoren-9-yl)methoxy)carbonyl)amino)-3-(6-(o-tolyl)pyridin-3-yl)propanoic acid

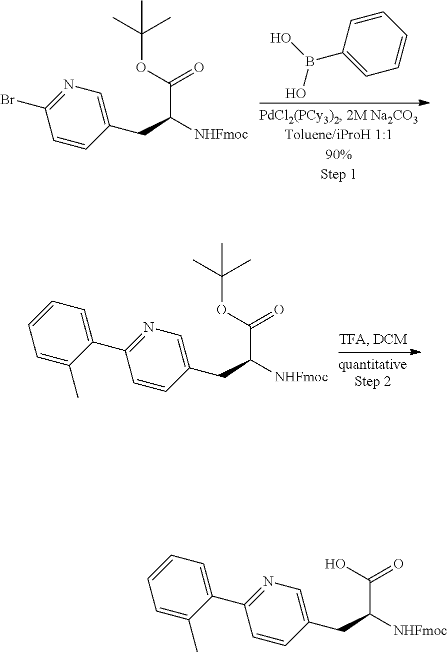

### Step 1

To a stirred solution of tert-butyl (S)-2-((((9H-fluoren-9-yl)methoxy)carbonyl)amino)-3-(6-bromopyridin-3-yl)propanoate (1750 mg, 3.35 mmol) in toluene/iPrOH (1:1, v:v, 50 mL) was added o-tolylboronic acid (911.6 mg, 6.7 mmol) and 2M Na₂CO₃ aqueous solution (25.0 mL). The mixture was purged with argon three times. Dichlorobis(tricyclohexylphosphine)palladium(II) (123.6 mg, 0.167 mmol) was added and the reaction mixture was purged twice with argon. The reaction was heated to 80° C. for 20 h. The reaction was cooled to room temperature and iPrOH was removed by rotovap. The crude was partitioned between water and EtOAc. The aqueous phase was extracted with EtOAc. Organic phases were combined and dried over anhydrous MgSO₄. After filtration and concentration the crude product was obtained as a brown oil. Purification by flash chromatography using EtOAc:DCM (1:9) as eluant lead to tert-butyl (S)-2-((((9H-fluoren-9-yl)methoxy)carbonyl)amino)-3-(6-(o-tolyl)pyridin-3-yl)propanoate (1.81 g, 3.39 mmol, 90%) as a colorless oil.

### Step 2

(S)-2-((((9H-Fluoren-9-yl)methoxy)carbonyl)amino)-3-(6-(o-tolyl)pyridin-3-yl)propanoate (1750 mg, 3.19 mmol) was dissolved in trifluoroacetic acid (5.00 mL) and the reaction was allowed to stir at room temperature for two hours. The reaction was brought to dryness on rotovap and the crude product was dissolved in diethyl ether and 1M HCl in diethyl ether. The mixture was sonicated for 2 hours to give a white solid. The product was isolated by filtration and washed with water to give (S)-2-((((9H-fluoren-9-yl)methoxy)carbonyl)amino)-3-(6-(o-tolyl)pyridin-3-yl)propanoic acid (1.91 g, 3.99 mmol, 100%) as a white solid. ¹H NMR (499 MHz, DMSO-d₆) δ 8.90 (s, 1H), 8.48 (br d, J=8.0 Hz, 1H), 7.96 (t, J=6.9 Hz, 2H), 7.89 (d, J=7.5 Hz, 2H), 7.64 (dd, J=7.2, 4.8 Hz, 2H), 7.52-7.45 (m, 1H), 7.43-7.29 (m, 7H), 4.46 (ddd, J=10.7, 8.9, 4.5 Hz, 1H), 4.25-4.15 (m, 3H), 3.45-3.34 (m, 1H), 3.18-3.10 (m, 1H), 3.08-3.00 (m, 1H), 2.27-2.20 (m, 3H).

### Preparation of (S)-2-((((9H-fluoren-9-yl)methoxy)carbonyl)amino)-3-(4′-acetamido-[11′-biphenyl]-4-yl)propanoic acid

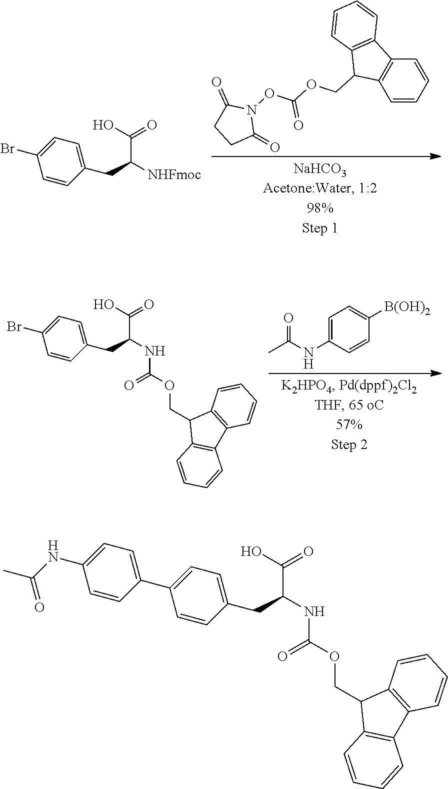

### Step 1

A 5.0-L multi-neck round-bottomed flask was charged with (S)-2-amino-3-(4-bromophenyl)propanoic acid (150.0 g, 615 mmol), Fmoc-OSu (207 g 615 mmol) in acetone (1500 mL), a solution of sodium bicarbonate (258 g, 3073 mmol) in water (3000 mL) in one lot and allowed to stir at room temperature for 16 h. The reaction mixture was slowly acidified with 10 N HCl solution to pH 1 and stirred for 15 min. The slurry was filtered and dried under vacuum and the cake was washed with water (3.0 L). Solids were dried for 16 h. The desired product was obtained as a white solid (280 g, 98%) and the product was taken to the next stage. Analysis condition E: Retention time=2.17 min; ESI-MS(+) m/z [M+H]⁺: 466.2.

### Step 2

To a stirred solution of (S)-2-((((9H-fluoren-9-yl)methoxy)carbonyl)amino)-3-(4-bromophenyl)propanoic acid (1.0 g, 2.144 mmol) and (4-acetamidophenyl)boronic acid (0.576 g, 3.22 mmol) with THF (50 mL) in 150-mL pressure tube, Argon was purged for 5 min. Potassium phosphate, tribasic (1.366 g, 6.43 mmol) was then added and the purging was continued for another 5 min. 1,1′-bis(di-tert-butylphosphino)ferrocene palladium dichloride (0.140 g, 0.214 mmol) was then added, and the purging was continued for another 5 min. The reaction mixture was heated to 65° C. for 26 h. The reaction mass was diluted with EtOAc (25 mL) and washed with 10% citric acid aqueous solution (10 mL) and then brine solution to get the crude product. It was triturated with 20% DCM, stirred for 10 min and filtered with a buchner funnel, and then dried for 10 min. The crude was purified by flash chromatography to give 0.7 g (57%) of the desired product as a brown solid. Analysis condition E: Retention time=1.79 min; ESI-MS(+) m/z [M+H]⁺: 519.0. ¹H NMR (400 MHz, DMSO-d₆) δ 12.75 (br s, 1H), 9.99 (s, 1H), 7.87 (d, J=7.5 Hz, 2H), 7.77-7.49 (m, 9H), 7.47-7.22 (m, 7H), 4.26-4.13 (m, 4H), 3.11 (br dd, J=13.8, 4.3 Hz, 1H), 2.91 (dd, J=13.8, 10.8 Hz, 1H), 2.12-2.01 (m, 4H).

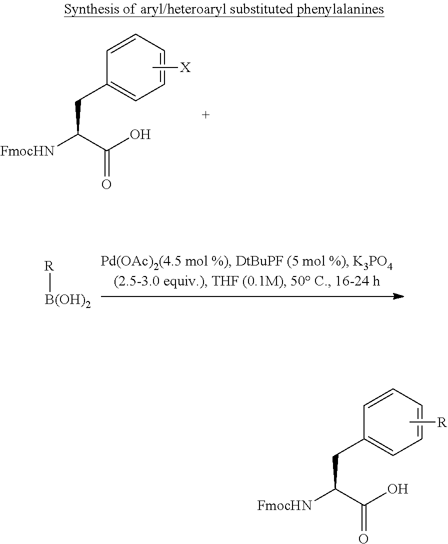

General procedures for Suzuki-Miyaura coupling (SMC) reactions in Scheme 1. To a N₂-flushed 20-mL scintillation vial equipped with a magnetic stir bar was added Fmoc-halo-Phe-OH (0.5 mmol), boronic acid (1.5-2.5 equiv.), and anhydrous THF (6 mL). The suspension was degassed by bubbling N₂ into the vial for several minutes. Palladium(II) acetate (4.5 mol %), DtBuPF (5 mol %), and then anhydrous K₃PO₄ (2.5 equiv.) were added. The suspension was degassed for several minutes, and then the vial was capped with a septum. The reaction mixture was stirred at 50° C. for 16 h. After cooling, 20% aqueous citric acid solution was added to acidify the reaction. The organic layer was separated, and the aqueous layer was extracted with EtOAc (2×). Silica gel was added to the combined organic layers, and the mixture was concentrated to dryness. The residue was dry-loaded on a silica gel column (ISCO system) and eluted with hexanes/EtOAc to give the desired product. Sometimes for compounds which are tailing in a Hexanes/EtOAc system, further eluting with MeOH/CH₂Cl₂ is also needed.

### Preparation of (S)-2-((((9H-fluoren-9-yl)methoxy)carbonyl)amino)-3-(4′-(tert-butoxycarbonyl)-[1,1′-biphenyl]-4-yl)propanoic acid

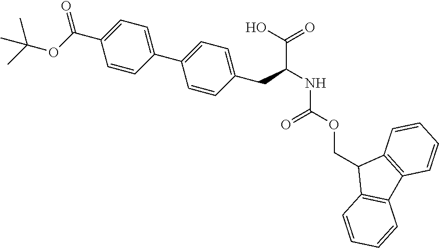 [SMILES: CC(C)(C)OC(=O)c1ccc(-c2ccc(C[C@H](NC(=O)OCC3c4ccccc4-c4ccccc43)C(=O)O)cc2)cc1]

(S)-2-((((9H-Fluoren-9-yl)methoxy)carbonyl)amino)-3-(4′-(tert-butoxycarbonyl)-[1,1′-biphenyl]-4-yl)propanoic acid was prepared according to the SMC general procedure. Yield: 78% (439 mg); colorless solids. ¹H NMR (400 MHz, methanol-d₄) δ 7.94 (d, J=8.3 Hz, 2H), 7.74 (d, J=7.6 Hz, 2H), 7.56 (d, J=8.4 Hz, 4H), 7.51 (d, J=8.1 Hz, 2H), 7.38-7.28 (m, 4H), 7.28-7.17 (m, 2H), 4.56-4.38 (m, 1H), 4.29 (dd, J=10.5, 7.0 Hz, 1H), 4.17 (dd, J=10.5, 7.1 Hz, 1H), 4.08 (t, J=7.0 Hz, 1H), 3.29-3.21 (m, 1H), 2.98 & 2.80 (dd, J=13.8, 9.6 Hz, total 1H), 1.59 (s, 9H). ESI-HRMS: Calcd for C₃₅H₃₄NO₆ [M+H]⁺ 564.23806, found 564.23896, mass difference 1.588 ppm.

### Preparation of (S)-2-((((9H-fluoren-9-yl)methoxy)carbonyl)amino)-3-(3′-(tert-butoxycarbonyl)-[1,1′-biphenyl]-4-yl)propanoic acid

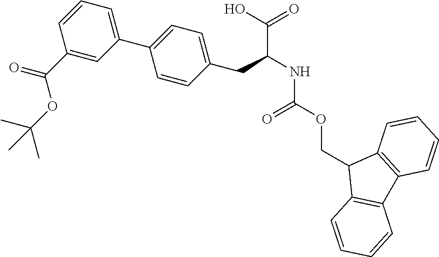 [SMILES: CC(C)(C)OC(=O)c1cccc(-c2ccc(C[C@H](NC(=O)OCC3c4ccccc4-c4ccccc43)C(=O)O)cc2)c1]

(S)-2-((((9H-Fluoren-9-yl)methoxy)carbonyl)amino)-3-(4′-(tert-butoxycarbonyl)-[1,1′-biphenyl]-4-yl)propanoic acid was prepared according to the SMC general procedure. Yield: 85% (240 mg); off-white solids. ¹H NMR (500 MHz, DMSO-d₆) δ 8.08 (t, J=1.8 Hz, 1H), 7.86 (dd, J=7.7, 1.4 Hz, 3H), 7.83 (d, J=8.1 Hz, 1H), 7.64 (d, J=7.7 Hz, 1H), 7.63 (d, J=7.5 Hz, 1H), 7.58-7.48 (m, 3H), 7.41-7.35 (m, 2H), 7.31 (d, J=7.8 Hz, 2H), 7.30-7.23 (m, 2H), 4.31-4.10 (m, 4H), 4.05 (td, J=8.2, 4.5 Hz, 1H), 3.13 & 2.9 (dd, J=13.6, 4.5 Hz, total 1H), 2.94 & 2.76 (dd, J=13.6, 8.7 Hz, total 1H), 1.56 (s, 9H). ESI-HRMS: Calcd for C₃₅H₃₇N₂O₆ [M+NH₄]⁺ 581.26461, found at 581.26474, mass difference 0.218 ppm.

### Preparation of(S)-2-((((9H-fluoren-9-yl)methoxy)carbonyl)amino)-3-(4-boronophenyl)propanoic acid

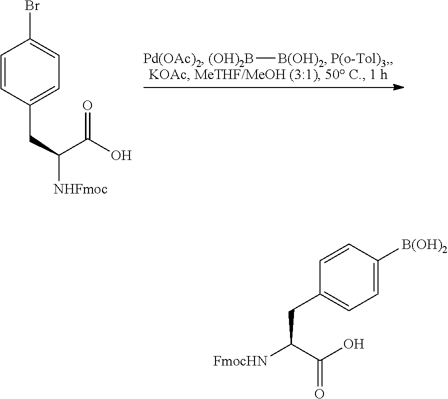

To a 75-mL pressure bottle (S)-2-((((9H-fluoren-9-yl)methoxy)carbonyl)amino)-3-(4-bromophenyl)propanoic acid (6.0 g, 12.87 mmol) and 2-methyl THF (250 mL) were charged, and the solution was purged with argon for 5 min. Tri-o-tolylphosphine (0.31 g, 1.03 mmol), tetrahydroxydiboron (2.31 g, 25.7 mmol), potassium acetate (3.79 g, 38.6 mmol) were added every in 10-min interval followed by the addition of MeOH (100 mL) and Pd(OAc)₂ (0.12 g, 0.52 mmol), and argon was purged for 10 min. The reaction was heated at 50° C. overnight. The reaction mixture was transferred into a 1-liter separatory funnel, diluted with 2-methyl-THF, and acidified with 1.5 N HCl to pH=2. The organic layer was washed with brine, dried (sodium sulphate), passed through celite, and concentrated to give black crude material. The crude was treated with petroleum ether to give a solid (10 g) which was dissolved with 2-methyl-THF and charcoal (2 g) was added. The mixture was heated on a rotovap without vacuum at 50° C. After filtration, the filtrate was passed through celite, concentrated. The resulting solid was treated with 30% ethyl acetate in petroleum ether, filtered to give 8 g of the crude as a fine off-white solid, which was further purified via flash chromatography then trituration with petroleum ether to give (S)-2-((((9H-fluoren-9-yl)methoxy)carbonyl)amino)-3-(4-boronophenyl)propanoic acid (4.0 g, 9.28 mmol, 72.1% yield) as a white solid. LCMS: 432.1 (M+H), tr=0.82 min. ¹H NMR (500 MHz, DMSO-d₆) δ 7.88 (d, J=7.6 Hz, 2H), 7.85-7.77 (m, 1H), 7.71 (br d, J=7.9 Hz, 3H), 7.68-7.60 (m, 2H), 7.41 (br d, J=6.6 Hz, 2H), 7.35-7.20 (m, 4H), 4.30-4.11 (m, 5H), 3.16-3.03 (m, 1H), 2.95-2.83 (m, 1H).

### Preparation of (S)-2-((((9H-fluoren-9-yl)methoxy)carbonyl)amino)-3-(4′-fluoro-[1,1′-biphenyl]-4-yl)propanoic acid

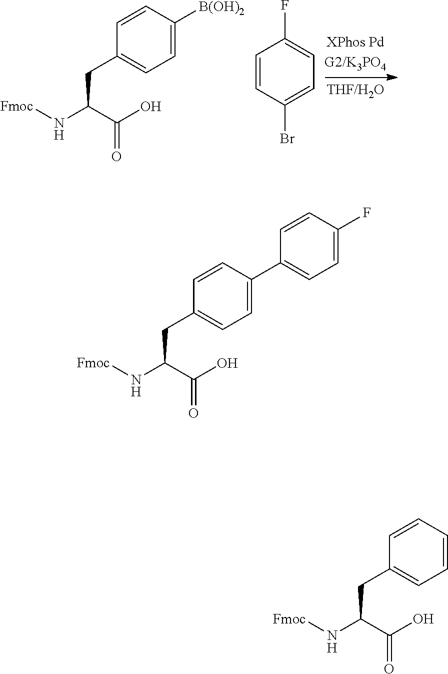

To a stirred solution of (S)-2-((((9H-fluoren-9-yl)methoxy)carbonyl)amino)-3-(4-boronophenyl)propanoic acid (217.5 mg, 0.504 mmol), 1-bromo-4-fluorobenzene (0.083 mL, 0.757 mmol) and XPhos Pd G2 (9.7 mg, 0.012 mmol) in THF (1 mL) at RT was added 0.5 M aqueous K₃PO₄ (2 mL, 1.000 mmol). N₂ was purged with vacuum three times and the mixture was stirred at 80° C. for 16 h. The mixture was cooled to RT. To the reaction was added 10% citric acid until pH<6. It was partitioned between EtOAc and H₂O, and the organic phase was separated, washed with brine, and dried over sodium sulfate. The mixture was filtered, SiO₂ (5 g) was added and concentrated. The material was then purified by flash chromatography (Teledyne ISCO CombiFlash Rr, gradient of 0% to 20% MeOH/CH₂Cl₂ over 15 column volumes, RediSep SiO₂ 40 g). Fractions containing the desired product were collected and concentrated to give (S)-2-((((9H-fluoren-9-yl)methoxy)carbonyl)amino)-3-(4′-fluoro-[1,1′-biphenyl]-4-yl)propanoic acid (206.1 mg, 0.43 mmol, 85% yield) as a cream solid: HPLC: RT=1.04 min (Waters Acquity UPLC BEH C18 1.7 um 2.1×50 mm, CH₃CN/H₂O/0.05% TFA, 1 min. gradient, wavelength=254 nm); MS (ES): m/z=482 [M+H]⁺. ¹H NMR (499 MHz, DMSO-d₆) δ 12.78 (br s, 1H), 7.88 (d, J=7.5 Hz, 3H), 7.71-7.61 (m, 5H), 7.53 (d, J=8.1 Hz, 2H), 7.39 (q, J=7.3 Hz, 3H), 7.36-7.23 (m, 8H), 4.24-4.13 (m, 5H), 3.12 (dd, J=14.0, 4.5 Hz, 1H), 2.91 (dd, J=13.6, 10.3 Hz, 1H).

### Preparation of (S)-2-((((9H-fluoren-9-yl)methoxy)carbonyl)amino)-3-(3′,5′-d fluoro-[1,1′-biphenyl]-4-yl)propanoic acid

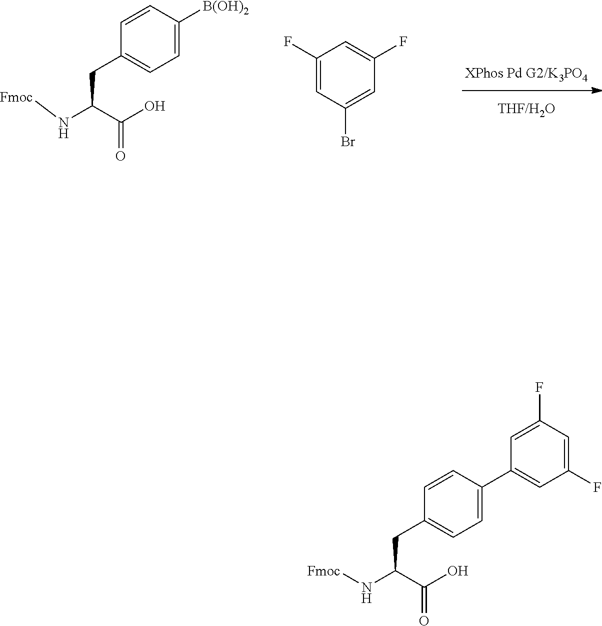

The final product was obtained following the same procedure of (S)-2-((((9H-fluoren-9-yl)methoxy)carbonyl)amino)-3-(4′-fluoro-[1,1′-biphenyl]-4-yl)propanoic acid. The Suzuki coupling reaction afforded the desired (S)-2-((((9H-fluoren-9-yl)methoxy)carbonyl)amino)-3-(3′,5′-difluoro-[1,1′-biphenyl]-4-yl)propanoic acid (197.1 mg, 0.40 mmol, 78% yield) as a colorless solid after purification by flash chromatography. HPLC: RT=1.06 min (Waters Acquity UPLC BEH C18 1.7 um 2.1×50 mm, CH₃CN/H₂O/0.05% TFA, 1 min. gradient, wavelength=254 nm); MS (ES): m/z=500 [M+H]⁺. ¹H NMR (499 MHz, DMSO-d₆) δ 12.90-12.67 (m, 1H), 7.87 (d, J=7.5 Hz, 2H), 7.69-7.61 (m, 4H), 7.45-7.35 (m, 6H), 7.33-7.27 (m, 2H), 7.22-7.16 (m, 1H), 4.25-4.18 (m, 3H), 4.17-4.12 (m, 1H), 3.14 (dd, J=13.8, 4.4 Hz, 1H), 2.92 (dd, J=13.7, 10.6 Hz, 1H).

### Preparation of (S)-2-((((9H-fluoren-9-yl)methoxy)carbonyl)amino)-3-(3′,4′,5′-trifluoro-[1,1′-biphenyl]-4-yl)propanoic acid

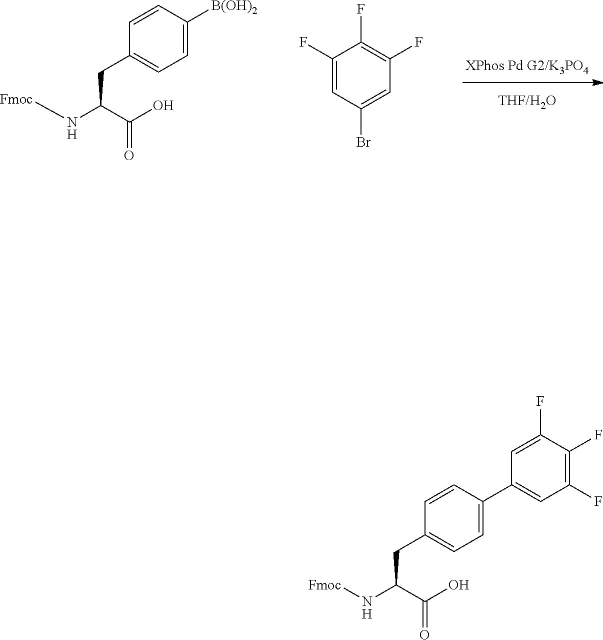

The final product was obtained following the same procedure of (S)-2-((((9H-fluoren-9-yl)methoxy)carbonyl)amino)-3-(4′-fluoro-[1,1′-biphenyl]-4-yl)propanoic acid. The Suzuki coupling reaction afforded the desired (S)-2-((((9H-fluoren-9-yl)methoxy)carbonyl)amino)-3-(3′,4,5′-trifluoro-[, 1′-biphenyl]-4-yl)propanoicacid (218.5 mg, 0.422 mmol, 84% yield) as a colourless solid after purification by flash chromatography. HPLC: RT=1.466 min (Shimadzu UPLC with Waters Acquity BEH C18 1.7 um 2.1×50 mm column, CH₃CN/H₂O/0.1% TFA, 3 min. gradient, wavelength=254 nm); MS (ES): m/z=556. ¹H NMR (499 MHz, DMSO-d₆) δ 12.79 (br s, 1H), 7.87 (d, J=7.6 Hz, 2H), 7.75 (d, J=8.6 Hz, 1H), 7.69-7.58 (m, 6H), 7.44-7.35 (m, 4H), 7.33-7.25 (m, 2H), 4.27-4.17 (m, 3H), 4.17-4.10 (m, 1H), 3.14 (dd, J=13.8, 4.4 Hz, 1H), 2.92 (dd, J=13.7, 10.7 Hz, 1H).

### General Procedure for Photoredox Reaction.

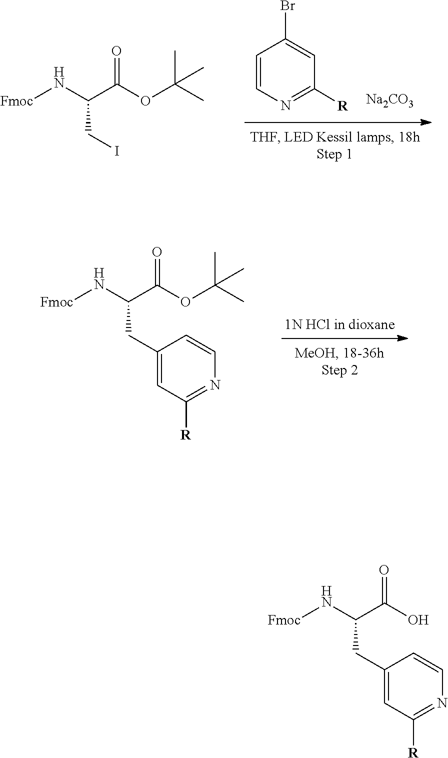

Ir[dF(CF₃)ppy₂]₂(dtbbpy)PF₆ (0.018 g, 0.016 mmol, 1 mol %), tert-butyl (R)-2-((((9H-fluoren-9-yl)methoxy)carbonyl)amino)-3-iodopropanoate (1.181 g, 2.393 mmol, 1.5 equiv), bromo-pyridine derivative (1.596 mmol, 1.00 equiv), pulverized Na₂CO₃ (0.338 g, 3.19 mmol, 2.00 equiv), and tris(trimethylsilane)silane (0.278 g. 1.596 mmol, 1.00 equiv) were charged into an oven-dried 40-mL pressure-relief screw cap vial. The vial was capped, purged with nitrogen, diluted with THF (45.0 mL), and then sonicated. In a separate vial were charged NiCl₂-glyme (18 mg, 0.080 mmol, 5 mol %) and di-tertbutylbipyridine (18 mg, 0.096 mmol, 6 mol %) in 1 mL dioxane. The vial was purged with nitrogen for 10 min. The Nickel-ligand complex solution was transferred to the main reaction vial and the mixture was degassed with gentle nitrogen flow for 20 min. The reactor was sealed with parafilm and placed between 2 34 W blue LED Kessil lamps (ca. 7 cm away) and allowed to stir vigorously. After 16 h, the reaction was monitored by LCMS analysis. The resulting oil was dissolved into 4 M HCl dioxane solution (15 mL). After 16 h, the reaction mixture was brought to dryness on a rotovap. The crude product was dissolved in a minimum amount of methanol and dry loaded on a silica gel column for purification.

### Preparation of (2S)-2-({[(9H-fluoren-9-yl)methoxy]carbonyl}amino)-3-(2-methoxypyridin-4-yl)propanoic acid

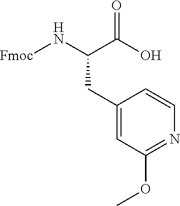

The mixture was rotovaped onto silica gel, purified by ISCO using 10% to 80% EtOAc/Hexanes. The fractions were pooled, concentrated to obtain the desired product as a clear oil (237 mg, 100%) Analysis conditions D: Retention time 1.74 min; ES+ 475.1.

### Preparation of ((S)-2-((((9H-fluoren-9-yl)methoxy)carbonyl)amino)-3-(4-(trifluoromethoxy)phenyl)propanoic acid

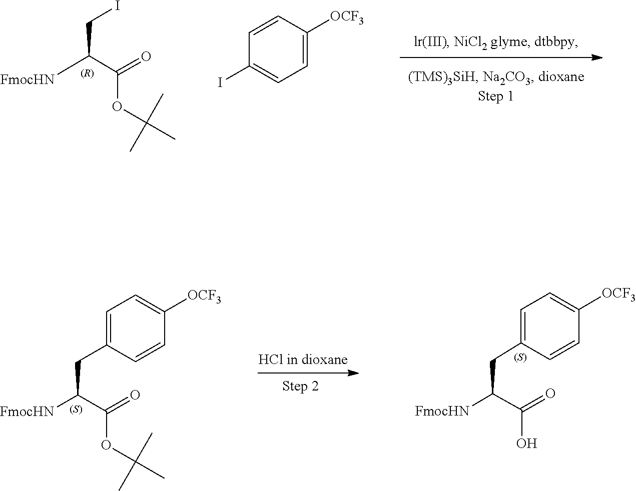

### Step 1

In 4 separate 40-mL vials was placed Ir(dF(CF₃)ppy)₂(dtbbpy)PF₆ (5.6 mg, 4.99 μmol) and Na₂CO₃ (249 mg, 2.35 mmol) in dioxane (18 mL), and each vial was fitted with a teflon screw cap and a stir bar. To the mixture was added 1-iodo-4-(trifluoromethoxy)benzene (0.16 mL, 1.02 mmol), stirred briefly, then tris(trimethylsilyl)silane (0.23 mL, 0.75 mmol) was added via syringe, and the suspension was degassed (cap on) with nitrogen for 5 min. To a separate 40-mL vial was added nickel(II) chloride ethylene glycol dimethyl ether complex (22 mg, 0.10 mmol) and 4,4′-di-tert-butyl-2,2′-bipyridine (33 mg, 0.12 mmol). Dioxane (10 mL) was added and this solution was degassed (cap on) with nitrogen gas for 10 min and stirred. To the Ir mixture was added 2.5 mL of the Ni solution, and 5 mL of a solution of the iodo alanine, tert-butyl (R)-2-((((9H-fluoren-9-yl)methoxy)carbonyl)amino)-3-iodopropanoate (987 mg, 2.0 mmol) in dioxane (20 mL), and then the mixture was further degassed with nitrogen gas for another 5 min (cap on). The vials were sealed with parafilm, placed in the round photoredox reactor with light and fan on, stirred for 40 h. The reactions were removed from the illumination/reactor. The blackish reaction mixtures of each vial were poured into a 500-mL erlenmeyer flask into which was added EtOAc (200 mL). The mixture was filtered through celite, washed with EtOAc, and concentrated. The residue was purified by flash chromatography (Teledyne ISCO CombiFlash Rf, gradient of 0% using solvent A/B=CH₂Cl₂/EtOAc over 10 column volumes, RediSep SiO2 80 g, loaded as DCM solution). The fractions containing the desired product were collected and concentrated to obtained the product tert-butyl (S)-2-((((9H-fluoren-9-yl)methoxy)carbonyl)amino)-3-(4-(trifluoromethoxy)phenyl)propanoate (865.2 mg, 1.64 mmol, 82% yield, only about 73% HPLC purity as a colourless oil and was used as is in the deprotection step: HPLC: RT=1.62 min (Waters Acquity UPLC BEH C18 1.7 um 2.1×50 mm, CH₃CN/H₂O/0.05% TFA, 1 min. gradient, wavelength=254 nm); MS (ES): m/z=550 [M+23]⁺

### Step 2

To a stirred solution of tert-butyl (S)-2-((((9H-fluoren-9-yl)methoxy)carbonyl)amino)-3-(4-(trifluoromethoxy)phenyl)propanoate (865.2 mg, 1.64 mmol) in dichloromethane (8.2 mL) at RT was added HCl (4M in dioxane, 8.20 mL, 32.8 mmol). The mixture was stirred at RT for 18 h. The mixture was concentrated in vacuo then dried under vacuum. The residue was dissolved in DMF (4 mL), purified on ISCO ACCQ Prep over 2 injections. The fractions containing the desired product were combined and partially concentrated on a rotovap, then blown air over mixture over weekend. The residue was dissolved in CH₃CN, diluted with water, frozen, and lyophilized to obtained the product (S)-2-((((9H-fluoren-9-yl)methoxy)carbonyl)amino)-3-(4-(trifluoromethoxy)phenyl)propanoic acid (344.1 mg, 0.73 mmol, 44.5% yield) as a colorless solid. HPLC: RT=1.38 min (Waters Acquity UPLC BEH C18 1.7 um 2.1×50 mm, CH₃CN/H₂O/0.05% TFA, 1.5 min. gradient, wavelength=254 nm); MS (ES): m/z=472 [M+1]+, ¹H NMR (499 MHz, DMSO-d₆) ppm 6 7.88 (d, J=7.5 Hz, 2H), 7.63 (d, J=7.4 Hz, 2H), 7.44-7.37 (m, 2H), 7.35-7.25 (m, 4H), 7.19 (br d, J=7.6 Hz, 3H), 4.30-4.20 (m, 1H), 4.21-4.13 (m, 2H), 4.04 (br d, J=3.5 Hz, 1H), 3.11 (br dd, J=13.6, 4.4 Hz, 1H), 2.91 (br dd, J=13.6, 9.1 Hz, 1H).

### Preparation of(S)-2-((((9H-fluoren-9-yl)methoxy)carbonyl)amino)-3-(2,5-dimethylphenyl)propanoic acid

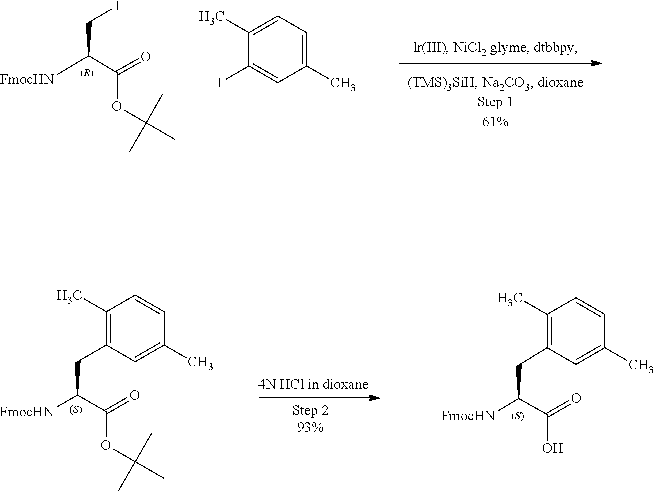

### Step 1

Compound was prepared following the same procedure of tert-butyl (S)-2-((((9H-fluoren-9-yl)methoxy)carbonyl)amino)-3-(4-(trifluoromethoxy)phenyl)propanoate. The photoredox coupling afforded the desired product, tert-butyl (S)-2-((((9H-fluoren-9-yl)methoxy)carbonyl)amino)-3-(2,5-dimethylphenyl)propanoate (140.5 mg, 0.298 mmol, 61.1% yield) after purification by flash chromatography. HPLC: RT=1.21 min (Waters Acquity UPLC BEH C18 1.7 um 2.1×50 mm, CH₃CN/H₂O/0.05% TFA, 1 min. gradient, wavelength=254 nm); Analysis condition F: Retention time=1.21 min; ESI-MS(+) m/z [M−tBu+H]⁺: 416. ¹H NMR (499 MHz, CHLOROFORM-d) δ 7.78 (d, J=7.5 Hz, 2H), 7.63-7.56 (m, 2H), 7.42 (t, J=7.4 Hz, 2H), 7.37-7.30 (m, 2H), 7.07 (d, J=7.7 Hz, 1H), 6.98 (d, J=7.7 Hz, 1H), 6.96 (s, 1H), 4.58-4.51 (m, 1H), 4.39 (dd, J=10.5, 7.3 Hz, 1H), 4.34 (dd, J=10.5, 7.2 Hz, 1H), 4.24-4.19 (m, 1H), 3.10-3.01 (m, 2H), 2.34 (s, 3H), 2.28 (s, 3H), 1.40 (s, 8H)

### Step 2

Final product was obtained following the same procedure of (S)-2-((((9H-fluoren-9-yl)methoxy)carbonyl)amino)-3-(4-(trifluoromethoxy)phenyl)propanoic acid. Removal of tBu ester with HCl/dioxane afforded the desired (S)-2-((((9H-fluoren-9-yl)methoxy)carbonyl)amino)-3-(2,5-dimethylphenyl)propanoic acid (115.2 mg, 0.277 mmol, 93% yield) as a cream solid after purification by reverse phase flash chromatography. HPLC: RT=1.03 min (Waters Acquity UPLC BEH C18 1.7 um 2.1×50 mm, CH₃CN/H₂O/0.05% TFA, 1 min. gradient, wavelength=254 nm); MS (ES): m/z=416 [M+H]⁺. ¹H NMR (499 MHz, CHLOROFORM-d) δ 7.88 (d, J=7.4 Hz, 2H), 7.79 (br d, J=8.6 Hz, 1H), 7.67 (d, J=7.4 Hz, 1H), 7.64 (d, J=7.5 Hz, 1H), 7.41 (td, J=7.3, 4.2 Hz, 3H), 7.35-7.29 (m, 2H), 7.29-7.25 (m, 1H), 7.02 (br d, J=8.9 Hz, 2H), 6.91 (br d, J=7.4 Hz, 1H), 4.21-4.10 (m, 5H), 3.07 (dd, J=14.1, 4.4 Hz, 1H), 2.80 (dd, J=14.1, 10.3 Hz, 1H), 2.24 (s, 3H), 2.18 (s, 3H)

### Preparation of(S)-2-((((9H-fluoren-9-yl)methoxy)carbonyl)amino)-3-(4-fluoro-3-methylphenyl)propanoic acid

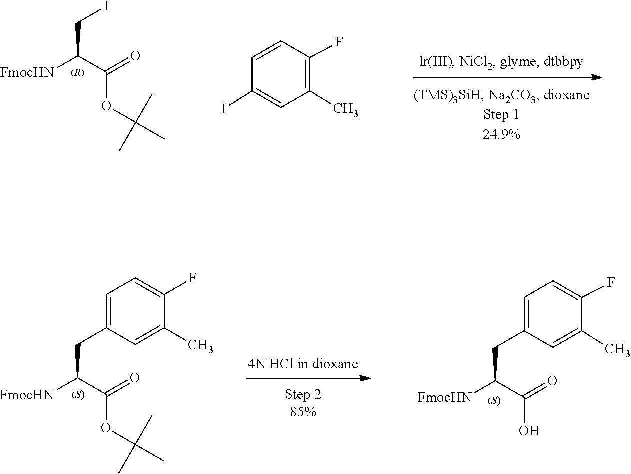

### Step 1

The compound was prepared following the same procedure of tert-butyl (S)-2-((((9H-fluoren-9-yl)methoxy)carbonyl)amino)-3-(4-(trifluoromethoxy)phenyl)propanoate. The photoredox coupling afforded the desired product, tert-butyl (S)-2-((((9H-fluoren-9-yl)methoxy)carbonyl)amino)-3-(4-fluoro-3-(trifluoromethyl)phenyl)propanoate (66.3 mg, 0.13 mmol, 24.9% yield) as a colourless solid after purification by flash chromatography. HPLC: RT=1.19 min (Waters Acquity UPLC BEH C18 1.7 um 2.1×50 mm, CH₃CN/H₂O/0.05% TFA, 1 min. gradient, wavelength=254 nm); MS (ES): m/z=474 [M-tBu]*. ¹H NMR (499 MHz, CHLOROFORM-d) δ 7.80 (d, J=7.5 Hz, 2H), 7.60 (dd, J=7.6, 3.3 Hz, 2H), 7.47-7.39 (m, 3H), 7.38-7.32 (m, 2H), 7.16-7.09 (m, 1H), 5.34 (br d, J=7.7 Hz, 1H), 4.57-4.47 (m, 2H), 4.40 (dd, J=10.3, 6.9 Hz, 1H), 4.26-4.21 (m, 1H), 3.14 (br d, J=4.9 Hz, 2H), 1.44 (s, 9H) Step 2

Final product was obtained following the same procedure of (S)-2-((((9H-fluoren-9-yl)methoxy)carbonyl)amino)-3-(4-(trifluoromethoxy)phenyl)propanoic acid. Removal of the tBu ester with HCl/dioxane afforded the desired (S)-2-((((9H-fluoren-9-yl)methoxy)carbonyl)amino)-3-(4-fluoro-3-methylphenyl)propanoic acid (58.3 mg, 0.139 mmol, 85% yield) as a cream solid after purification by reverse phase flash chromatography. HPLC: RT=1.02 min (Waters Acquity UPLC BEH C18 1.7 um 2.1×50 mm, CH₃CN/H₂O/0.05% TFA, 1 min. gradient, wavelength=254 nm); MS (ES): m/z=420 [M+H]⁺. ¹H NMR (499 MHz, DMSO-d₆) δ 12.86-12.66 (m, 1H), 7.89 (d, J=7.5 Hz, 2H), 7.73 (d, J=8.3 Hz, 1H), 7.65 (t, J=7.5 Hz, 2H), 7.42 (t, J=7.5 Hz, 2H), 7.35-7.26 (m, 2H), 7.17 (br d, J=7.5 Hz, 1H), 7.14-7.08 (m, 1H), 7.06-6.99 (m, 1H), 4.24-4.11 (m, 4H), 3.03 (dd, J=13.7, 4.3 Hz, 1H), 2.82 (dd, J=13.6, 10.6 Hz, 1H), 2.17 (s, 3H).

### Preparation of (S)-2-((((9H-fluoren-9-yl)methoxy)carbonyl)amino)-3-(2,4-difluoro-5-methoxyphenyl)propanoic acid

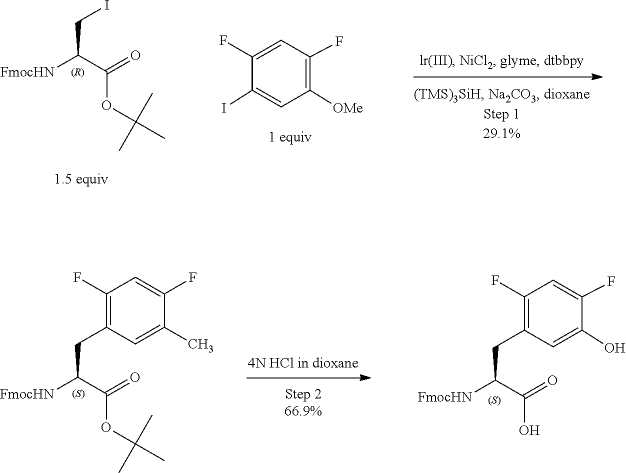

### Step 1

The compound was prepared following the same procedure of tert-butyl (S)-2-((((9H-fluoren-9-yl)methoxy)carbonyl)amino)-3-(4-(trifluoromethoxy)phenyl)propanoate. The photoredox coupling afforded the desired product, tert-butyl (S)-2-((((9H-fluoren-9-yl)methoxy)carbonyl)amino)-3-(2,4-d fluoro-5-methoxyphenyl)propanoate (77.1 mg, 0.151 mmol, 29.1% yield as a colourless solid after purification by flash chromatography. HPLC: RT=1.15 min (Waters Acquity UPLC BEH C18 1.7 um 2.1×50 mm, CH₃CN/H₂O/0.05% TFA, 1 min. gradient, wavelength=254 nm); MS (ES): m/z=454 [M-t-Bu]+. ¹H NMR (499 MHz, CHLOROFORM-d) δ 7.79 (d, J=7.4 Hz, 2H), 7.59 (t, J=6.4 Hz, 2H), 7.43 (t, J=7.3 Hz, 2H), 7.33 (td, J=7.5, 1.1 Hz, 3H), 6.85 (dd, J=10.8, 9.3 Hz, 1H), 6.83-6.79 (m, 1H), 5.40 (br d, J=8.1 Hz, 1H), 4.58-4.51 (m, 1H), 4.38 (dd, J=7.0, 4.5 Hz, 2H), 4.25-4.20 (m, 1H), 3.82 (s, 3H), 3.18-3.05 (m, 2H), 1.45 (s, 9H)

### Step 2

The final product was obtained following the same procedure of (S)-2-((((9H-fluoren-9-yl)methoxy)carbonyl)amino)-3-(4-(trifluoromethoxy)phenyl)propanoic acid. Removal of tBu ester with HCl/dioxane afforded the desired (S)-2-((((9H-fluoren-9-yl)methoxy)carbonyl)amino)-3-(2,4-difluoro-5-methoxyphenyl)propanoic acid (45.9 mg, 0.101 mmol, 66.9% yield) as a cream solid after purification by reverse phase flash chromatography. HPLC: RT=0.99 min (Waters Acquity UPLC BEH C18 1.7 um 2.1×50 mm, CH₃CN/H₂O/0.05% TFA, 1 min. gradient, wavelength=254 nm); MS (ES): m/z=454 [M+1]+. ¹H NMR (499 MHz, DMSO-d₆) δ 12.92 (br s, 1H), 7.89 (d, J=7.5 Hz, 2H), 7.71-7.65 (m, 1H), 7.63 (d, J=7.5 Hz, 2H), 7.41 (t, J=7.5 Hz, 2H), 7.34-7.25 (m, 2H), 7.24-7.15 (m, 2H), 4.24-4.12 (m, 4H), 3.77 (s, 3H), 3.16 (br dd, J=13.8, 4.6 Hz, 1H), 2.82 (dd, J=13.6, 10.7 Hz, 1H). Preparation of(S)-2-((((9H-fluoren-9-yl)methoxy)carbonyl)amino)-3-(2,3-dimethylphenyl)propanoic acid

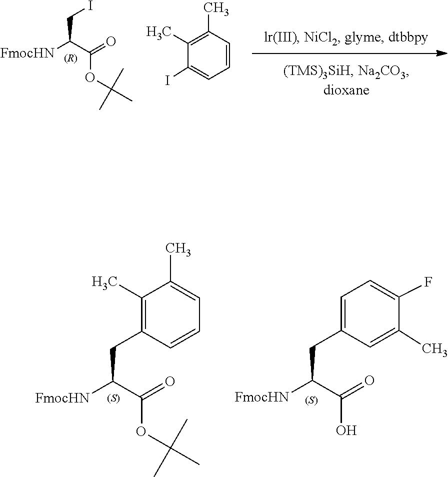

### Step 1

The compound was prepared following the same procedure of tert-butyl (S)-2-((((9H-fluoren-9-yl)methoxy)carbonyl)amino)-3-(4-(trifluoromethoxy)phenyl)propanoate. The photoredox coupling afforded the desired product, tert-butyl (S)-2-((((9H-fluoren-9-yl)methoxy)carbonyl)amino)-3-(2,3-dimethylphenyl)propanoate (107.5 mg, 0.228 mmol, 55.5% yield) as a tan viscous oil after purification by flash chromatography. HPLC: RT=1.21 min (Waters Acquity UPLC BEH C18 1.7 um 2.1×50 mm, CH₃CN/H₂O/0.05% TFA, 1 min. gradient, wavelength=254 nm); MS (ES): m/z=416 [M-t-Bu]+. ¹H NMR (499 MHz, CHLOROFORM-d) δ 7.79 (d, J=7.5 Hz, 2H), 7.61-7.56 (m, 2H), 7.42 (t, J=7.5 Hz, 2H), 7.35-7.31 (m, 2H), 7.09-7.06 (m, 1H), 7.02 (t, J=7.5 Hz, 1H), 7.00-6.96 (m, 1H), 5.30 (br d, J=8.3 Hz, 1H), 4.53 (q, J=7.4 Hz, 1H), 4.39 (dd, J=10.6, 7.3 Hz, 1H), 4.34 (dd, J=10.4, 7.0 Hz, 1H), 4.21 (t, J=7.2 Hz, 1H), 3.15 (dd, J=14.2, 7.0 Hz, 1H), 3.08 (dd, J=14.1, 7.3 Hz, 1H), 2.29 (s, 3H), 2.28 (s, 3H), 1.40 (s, 9H).

### Step 2

The final product was obtained following the same procedure of (S)-2-((((9H-fluoren-9-yl)methoxy)carbonyl)amino)-3-(4-(trifluoromethoxy)phenyl)propanoic acid. Removal of the tBu ester with HCl/dioxane afforded the desired (S)-2-((((9H-fluoren-9-yl)methoxy)carbonyl)amino)-3-(2,3-dimethylphenyl)propanoic acid (72.9 mg, 0.175 mmol, 77% yield) as a cream solid after purification by reverse phase flash chromatography. HPLC: RT=1.03 min (Waters Acquity UPLC BEH C18 1.7 um 2.1×50 mm, CH₃CN/H₂O/0.05% TFA, 1 min. gradient, wavelength=254 nm); MS (ES): m/z=416 [M+H]+. ¹H NMR (499 MHz, DMSO-d₆) δ 12.76 (br d, J=1.8 Hz, 1H), 7.89 (d, J=7.5 Hz, 2H), 7.79-7.71 (m, 1H), 7.66 (dd, J=13.6, 7.6 Hz, 2H), 7.42 (td, J=7.2, 4.1 Hz, 2H), 7.35-7.27 (m, 2H), 7.07 (d, J=7.3 Hz, 1H), 7.04-6.99 (m, 1H), 6.99-6.94 (m, 1H), 4.24-4.14 (m, 3H), 4.13-4.05 (m, 1H), 3.15 (dd, J=14.1, 4.1 Hz, 1H), 2.85 (dd, J=13.9, 10.4 Hz, 1H), 2.22 (s, 3H), 2.19 (s, 3H).

### Preparation of(S)-2-((((9H-fluoren-9-yl)methoxy)carbonyl)amino)-3-(2-fluoro-3-methylphenyl)propanoic acid

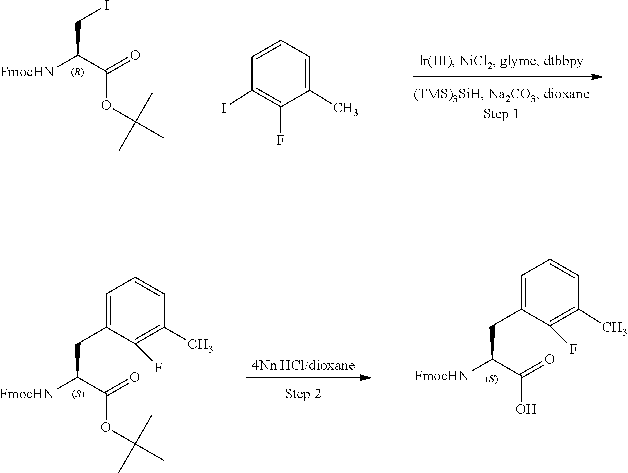

### Step 1

The compound was prepared following the same procedure of tert-butyl (S)-2-((((9H-fluoren-9-yl)methoxy)carbonyl)amino)-3-(4-(trifluoromethoxy)phenyl)propanoate. The photoredox coupling afforded the desired product, tert-butyl (S)-2-((((9H-fluoren-9-yl)methoxy)carbonyl)amino)-3-(2-fluoro-3-methylphenyl)propanoate (136.9 mg, LCMS showed 77% product and 23% impurity) as a viscous oil after purification by flash chromatography. Used as is, purified after tBu hydrolysis.

### Step 2

The final product was obtained following the same procedure of (S)-2-((((9H-fluoren-9-yl)methoxy)carbonyl)amino)-3-(4-(trifluoromethoxy)phenyl)propanoic acid. Removal of tBu ester with HCl/dioxane afforded the desired (S)-2-((((9H-fluoren-9-yl)methoxy)carbonyl)amino)-3-(2-fluoro-3-methylphenyl)propanoic acid (79.7 mg, 0.190 mmol, 66.0% yield) as a cream solid after purification by reverse phase flash chromatography. HPLC: RT=1.02 min (Waters Acquity UPLC BEH C18 1.7 um 2.1×50 mm, CH₃CN/H₂O/0.05% TFA, 1 min. gradient, wavelength=254 nm); MS (ES): m/z=420 [M+1]⁺. ¹H NMR (499 MHz, DMSO-d₆) δ 12.79 (br s, 1H), 7.89 (d, J=7.7 Hz, 2H), 7.78 (d, J=8.6 Hz, 1H), 7.65 (dd, J=1 1.6, 7.5 Hz, 2H), 7.44-7.39 (m, 3H), 7.37-7.25 (m, 3H), 7.14 (br t, J=7.4 Hz, 2H), 7.01-6.96 (m, 1H), 4.24-4.12 (m, 4H), 3.17 (dd, J=13.8, 4.8 Hz, 1H), 2.86 (dd, J=13.6, 10.8 Hz, 1H), 2.21 (s, 3H). ¹H NMR and LCMS showed a 14% impurity.

### Preparation of ((S)-2-((((9H-fluoren-9-yl)methoxy)carbonyl)amino)-3-(2-fluoro-S-methylphenyl)propanoic acid

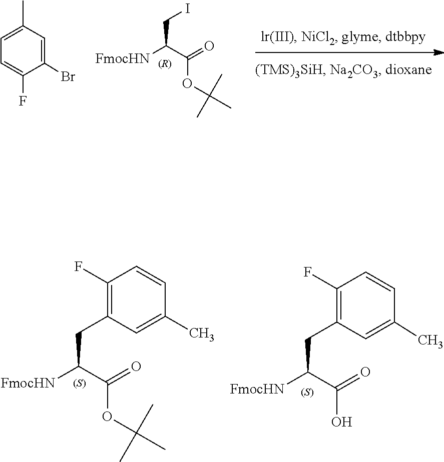

The compound was prepared following the same procedure of tert-butyl (S)-2-((((9H-fluoren-9-yl)methoxy)carbonyl)amino)-3-(4-(trifluoromethoxy)phenyl)propanoate. The photoredox coupling afforded the desired product, tert-butyl (S)-2-((((9H-fluoren-9-yl)methoxy)carbonyl)amino)-3-(2-fluoro-5-methylphenyl)propanoate (148.1 mg, 0.311 mmol, 65.4% yield) as a colourless gum after purification by flash chromatography. HPLC: RT=1.19 min (Waters Acquity UPLC BEH C18 1.7 um 2.1×50 mm, CH₃CN/H₂O/0.05% TFA, 1 min. gradient, wavelength=254 nm); MS (ES): m/z=420 [M-t-Bu]⁺. ¹H NMR (499 MHz, CHLOROFORM-d) δ 7.79 (d, J=7.6 Hz, 2H), 7.60 (t, J=7.2 Hz, 2H), 7.42 (t, J=7.4 Hz, 2H), 7.37-7.30 (m, 2H), 7.06-6.99 (m, 2H), 6.97-6.90 (m, 1H), 5.41 (br d, J=8.1 Hz, 1H), 4.60-4.54 (m, 1H), 4.43 (dd, J=10.4, 7.2 Hz, 1H), 4.30 (dd, J=10.1, 7.5 Hz, 1H), 4.26-4.21 (m, 1H), 3.16 (dd, J=13.9, 6.7 Hz, 1H), 3.10 (dd, J=13.9, 6.4 Hz, 1H), 2.28 (s, 3H), 1.44 (s, 9H).

### Step 2

The final product was obtained following the same procedure of (S)-2-((((9H-fluoren-9-yl)methoxy)carbonyl)amino)-3-(4-(trifluoromethoxy)phenyl)propanoic acid. Removal of tBu ester with HCl/dioxane afforded the desired (S)-2-((((9H-fluoren-9-yl)methoxy)carbonyl)amino)-3-(2-fluoro-5-methylphenyl)propanoic acid (98.1 mg, 0.23 mmol, 75% yield) as a colourless solid after purification by reverse phase flash chromatography. HPLC: RT=1.01 min (Waters Acquity UPLC BEH C18 1.7 um 2.1×50 mm, CH₃CN/H₂O/0.05% TFA, 1 min. gradient, wavelength=254 nm); MS (ES): m/z=420 [M+1]⁺. ¹H NMR (499 MHz, DMSO-d₆) δ 12.82 (br s, 1H), 7.89 (d, J=7.5 Hz, 2H), 7.78 (d, J=8.6 Hz, 1H), 7.67 (d, J=7.4 Hz, 1H), 7.64 (d, J=7.4 Hz, 1H), 7.42 (td, J=7.4, 3.0 Hz, 2H), 7.34-7.27 (m, 2H), 7.16-7.11 (m, 1H), 7.08-6.97 (m, 2H), 4.26-4.12 (m, 5H), 3.15 (dd, J=13.8, 4.9 Hz, 1H), 2.83 (dd, J=13.8, 10.3 Hz, 1H), 2.20 (s, 3H).

### Preparation of(S)-2-((((9H-fluoren-9-yl)methoxy)carbonyl)amino)-3-(2-fluoro-5-methoxyphenyl)propanoic acid

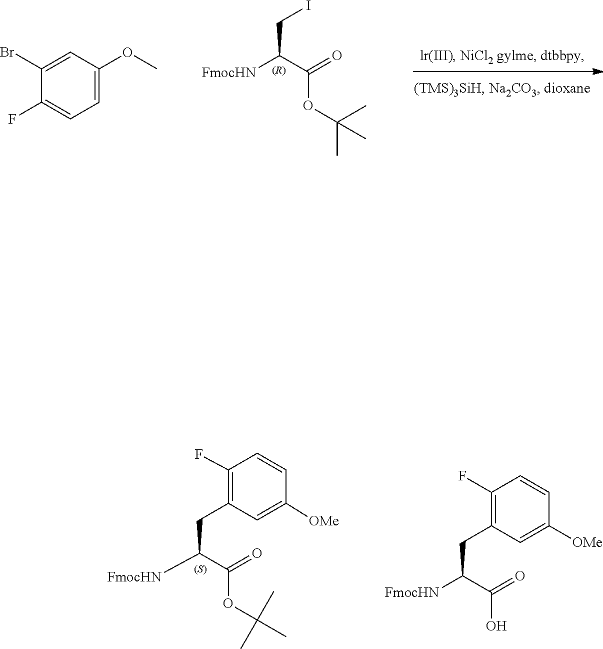

### Step 1

The compound was prepared following the same procedure of tert-butyl (S)-2-((((9H-fluoren-9-yl)methoxy)carbonyl)amino)-3-(2-fluoro-5-methoxyphenyl)propanoate (117.7 mg, 0.24 mmol, 50.4% yield) as a colourless solid after purification by flash chromatography. HPLC: RT=1.15 min (Waters Acquity UPLC BEH C18 1.7 um 2.1×50 mm, CH₃CN/H₂O/0.05% TFA, 1 min. gradient, wavelength=254 nm); MS (ES): m/z=436 [M-t-Bu]*.

¹H NMR (499 MHz, CHLOROFORM-d) δ 7.78 (d, J=7.5 Hz, 2H), 7.63-7.56 (m, 2H), 7.42 (t, J=7.4 Hz, 2H), 7.37-7.30 (m, 2H), 7.01-6.93 (m, 1H), 6.79-6.72 (m, 2H), 5.41 (br d, J=8.2 Hz, 1H), 4.62-4.55 (m, 1H), 4.41 (dd, J=10.4, 7.3 Hz, 1H), 4.31 (dd, J=10.5, 7.4 Hz, 1H), 4.26-4.20 (m, 1H), 3.75 (s, 3H), 3.17 (dd, J=13.9, 6.7 Hz, 1H), 3.11 (dd, J=14.4, 6.6 Hz, 1H), 1.45 (s, 9H).

### Step 2

The final product was obtained following the same procedure of (S)-2-((((9H-fluoren-9-yl)methoxy)carbonyl)amino)-3-(4-(trifluoromethoxy)phenyl)propanoic acid. Removal of tBu ester with HCl/dioxane afforded the desired (S)-2-((((9H-fluoren-9-yl)methoxy)carbonyl)amino)-3-(2-fluoro-S-methoxyphenyl)propanoic acid (79.5 mg, 0.183 mmol, 76% yield) as a colourless solid after purification by flash chromatography. HPLC: RT=0.98 min (Waters Acquity UPLC BEH C18 1.7 um 2.1×50 mm, CH₃CN/H₂O/0.05% TFA, 1 min. gradient, wavelength=254 nm); MS (ES): m/z=436 [M+1]+. Base peak of 214=fully deprotected amino acid fragment was also observed. ¹H NMR (499 MHz, DMSO-d₆) δ 12.84 (br s, 1H), 7.89 (d, J=7.5 Hz, 2H), 7.79 (d, J=8.6 Hz, 1H), 7.64 (t, J=8.4 Hz, 2H), 7.45-7.38 (m, 2H), 7.34-7.25 (m, 2H), 7.07 (t, J=9.2 Hz, 1H), 6.94 (dd, J=6.1, 3.2 Hz, 1H), 6.80 (dt, J=8.9, 3.6 Hz, 1H), 4.25-4.13 (m, 4H), 3.69 (s, 3H), 3.17 (dd, J=13.9, 4.6 Hz, 1H), 2.83 (dd, J=13.7, 10.7 Hz, 1H).

### Preparation of (S)-2-((((9H-fluoren-9-yl)methoxy)carbonyl)amino)-3-(2-methoxy-S-methylphenyl)propanoic acid

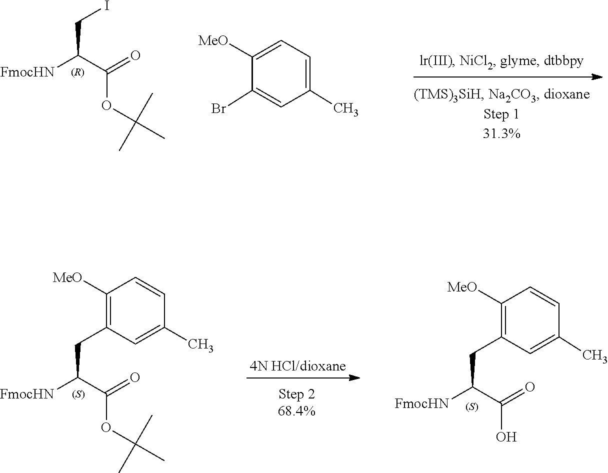

### Step 1

The compound was prepared following the same procedure of tert-butyl (S)-2-((((9H-fluoren-9-yl)methoxy)carbonyl)amino)-3-(4-(trifluoromethoxy)phenyl)propanoate. The photoredox coupling afforded the desired product, tert-butyl (S)-2-((((9H-fluoren-9-yl)methoxy)carbonyl)amino)-3-(2-methoxy-S-methylphenyl)propanoate (73.9 mg, 0.15 mmol, 31.3% yield) as a colourless film after purification by flash chromatography. HPLC: RT=1.20 min (Waters Acquity UPLC BEH C18 1.7 um 2.1×50 mm, CH₃CN/H₂O/0.05% TFA, 1 min. gradient, wavelength=254 nm); MS (ES): m/% z=488 [M−tBu+H]⁺. ¹H NMR (499 MHz, CHLOROFORM-d) δ 7.78 (d, J=7.6 Hz, 2H), 7.61-7.54 (m, 2H), 7.41 (t, J=7.4 Hz, 2H), 7.34-7.30 (m, 2H), 7.05 (dd, J=8.1, 1.5 Hz, 1H), 6.98 (d, J=1.4 Hz, 1H), 6.79 (d, J=8.3 Hz, 1H), 5.70 (br d, J=7.7 Hz, 1H), 4.49 (q, J=7.4 Hz, 1H), 4.33 (d, J=7.4 Hz, 2H), 4.25-4.18 (m, 1H), 3.82 (s, 3H), 3.10-3.02 (m, 2H), 2.26 (s, 3H), 1.43 (s, 9H) Step 2

The final product was obtained following the same procedure of (S)-2-((((9H-fluoren-9-yl)methoxy)carbonyl)amino)-3-(4-(trifluoromethoxy)phenyl)propanoic acid Removal of tBu ester with HCl/dioxane afforded the desired (S)-2-((((9H-fluoren-9-yl)methoxy)carbonyl)amino)-3-(2-methoxy-5-methylphenyl)propanoic acid (44.7 mg, 0.104 mmol, 68.4% yield) as a colourless solid after purification by flash chromatography. HPLC: RT=1.02 min (Waters Acquity UPLC BEH C18 1.7 um 2.1×50 mm, CH₃CN/H₂O/0.05% TFA, 1 min. gradient, wavelength=254 nm); MS (ES): m/z=432 [M+H]⁺. ¹H NMR (499 MHz, DMSO-d₆) δ 12.61 (br s, 1H), 7.89 (d, J=7.5 Hz, 2H), 7.67 (d, J=7.5 Hz, 1H), 7.63 (d, J=7.5 Hz, 1H), 7.60 (br d, J=8.1 Hz, 1H), 7.42 (td, J=7.2, 3.5 Hz, 2H), 7.32 (td, J=7.5, 1.0 Hz, 1H), 7.30-7.26 (m, 1H), 7.02-6.97 (m, 2H), 6.84 (d, J=8.9 Hz, 1H), 4.26-4.10 (m, 4H), 3.75 (s, 3H), 3.12 (dd, J/=13.5, 4.8 Hz, 1H), 2.72 (dd, J=13.4, 10.2 Hz, 1H), 2.16 (s, 3H).

### Preparation of (S)-2-((((9H-fluoren-9-yl)methoxy)carbonyl)amino)-3-hydroxy-3-methylbutanoic acid

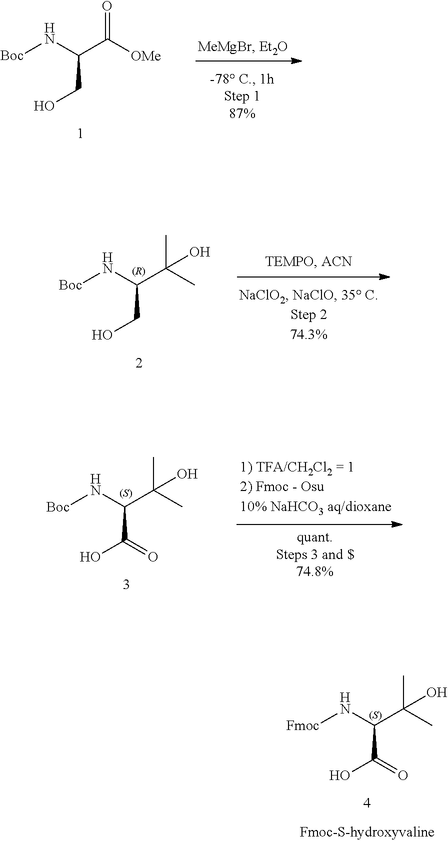

### Step 1

To a 10-L multi-neck round-bottomed flask was charged methyl (tert-butoxycarbonyl)-D-serinate (50 g, 228 mmol), diethyl ether (4200 mL). The mixture was cooled to −78° C. and methylmagnesium bromide (456 mL, 1368 mmol) was added dropwise over 30 min. The reaction was stirred at RT for 1 h. It was cooled to 0° C. and saturated NH₄Cl solution (1500 mL), was added dropwise and stirred for 10 min. The organic layer was separated and the aqueous layer was extracted with ethyl acetate (3×2000 mL). The combined organic layer was washed with brine, dried over Na₂SO₄, and concentrated at 40° C. to give a colorless thick liquid. The crude was purified by I2PAC. Desired fractions were eluted at 50% EtOAc:petroleum ether mixture, and were collected and concentrated at 40° C. to give tert-butyl (R)-(1,3-dihydroxy-3-methylbutan-2-yl)carbamate (43.5 g, 87%) as a white solid. ¹H NMR (MeOD, 300 MHz) δ 3.70 (m, 1H), 3.48 (m, 1H), 3.21 (m, 1H), 1.35 (s, 9H), 1.13 (s, 3H), 1.05 (s, 3H).

### Step 2

A 50-mL single neck round-bottomed flask was charged with tert-butyl (R)-(1,3-dihydroxy-3-methylbutan-2-yl)carbamate (43.0 g, 196 mmol), acetonitrile (650 mL) and was stirred till solution became clear. Sodium phosphate buffer (460 mL, 196 mmol) (pH=6.7, 0.67 M), (diacetoxyiodo)benzene (4.48 g, 13.92 mmol), and TEMPO (2.206 g, 14.12 mmol) were added sequentially and then the reaction was cooled to 0° C. and sodium chlorite (19.95 g, 221 mmol) was added. The color of the reaction turned black. The reaction was allowed to stir at 0° C. for 2 h. then at RT overnight. The orange colored reaction was quenched with saturated ammonium chloride solution (1000 mL) and the pH meter was used to adjust the pH=2 using 1.5 N HCl (330 mL). The aqueous solution was saturated with solid NaCl and extracted with ethyl acetate. The combined organic layer was washed with brine, dried over Na₂SO₄, and concentrated to obtain crude (S)-2-((tert-butoxycarbonyl)amino)-3-hydroxy-3-methylbutanoic acid (34.0 g, 74.3% yield) as an off-white solid and was taken directly to the next stage. ¹H NMR (MeOD, 300 MHz) δ 3.98 (s, 1H), 1.35 (s, 9H), 1.19 (s, 3H), 1.16 (9s, 3H).

### Step 3

A 2000-mL single neck flask was charged with (S)-2-((tert-butoxycarbonyl)amino)-3-hydroxy-3-methylbutanoic acid (90 g, 386 mmol) in dioxane (450 mL) and was cooled to 0° C. 4N HCl in Dioxane (450 mL, 1800 mmol) was added dropwise over 10 min. The reaction was allowed to stir at RT for 3 h. It was concentrated and azetroped with toluene (2×) then stirred with ethyl acetate for 10 min. It was filtered and dried under vacuum to obtain crude (S)-2-amino-3-hydroxy-3-methylbutanoic acid, HCl (70 g, 107% yield) as a white solid and was taken directly to the next step.

### Step 4

To a 3000-mL multi-neck round-bottomed flask was charged (S)-2-amino-3-hydroxy-3-methylbutanoic acid, HCl (70 g, 413 mmol), dioxane (1160 mL), and water (540 mL). The stirred solution became clear and a solution of sodium bicarbonate (104 g, 1238 mmol) in water (1160 mL) was added in one portion at RT. The reaction mass was allowed to stir at RT for 30 min. A solution of Fmoc-OSu (139 g, 413 mmol) in 1,4-dioxane (1460 mL) was added in one portion at RT. The reaction was allowed to stir at RT for 16 h. The reaction was concentrated to remove dioxane. To the resulting solution water was added and washed with ethyl acetate (3×1000 mL). The aqueous solution was acidified to pH 1-2 and extracted with ethyl acetate. The combined organic layer was washed with water, followed by brine, finally dried over Na₂SO₄, and concentrated to give an off-white solid (135.7 g). To remove the trapped dioxane and ethyl acetate the following proceture was followed: the solid was dissolved in ethyl acetate (1200 mL) and was stripped off with n-hexane (3000 mL). The slurry obtained was stirred for 10 min, filtered, dried under vacuum to give (S)-2-((((9H-fluoren-9-yl)methoxy)carbonyl)amino)-3-hydroxy-3-methylbutanoic acid (112.0 g, 74.8 yield for two steps) as a white solid.

### Preparation of(S)-2-((((9H-fluoren-9-yl)methoxy)carbonyl)amino)-3-(3,4,5-trifluorophenyl)propanoic acid

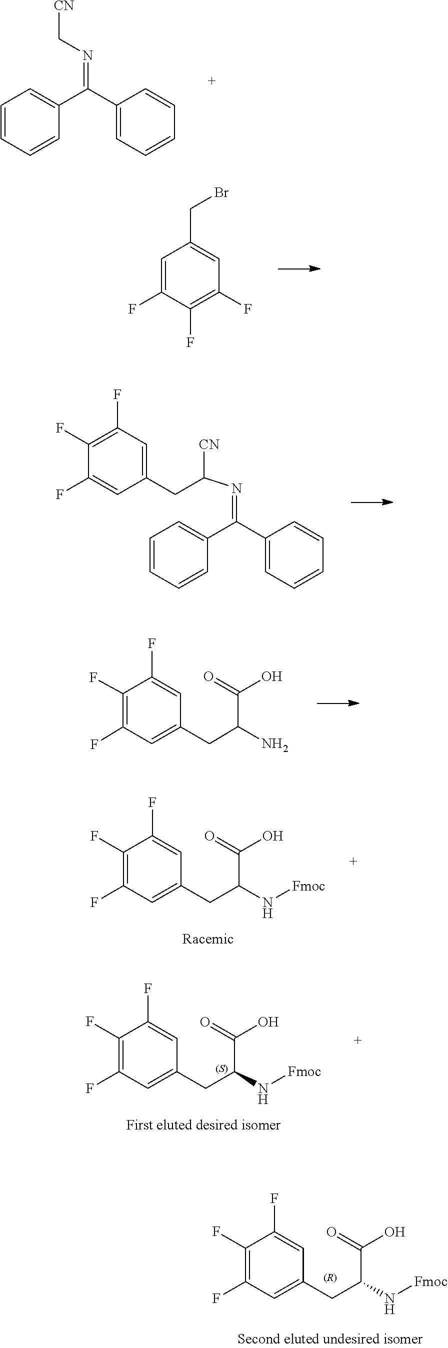

### Step 1

To a stirred solution of 2-((diphenylmethylene)amino)acetonitrile (100 g, 454 mmol) in DCM (1000 mL), 5-(bromomethyl)-1,2,3-trifluorobenzene (66.5 mL, 499 mmol) and benzyltrimethylammonium chloride (16.86 g, 91 mmol) was added. To this, 10 M NaOH (136 mL, 1362 mmol) solution was added and stirred at RT overnight. After 26 h, the reaction mixture was diluted with water (500 mL) and the DCM layer was separated. The aqeuous layer was further extracted with DCM (2×250 mL). The organic layer was combined, washed with water and brine solution, dried over Na₂SO₄, filtered, and concentrated under vacuum. The crude compound was purified by flash column chromatography (1.5 kg, silica gel, 0-10% ethylacetate/petroleum ether mixture) and the desired fractions were collected and concentrated to afford 2-((diphenylmethylene)amino)-3-(3,4,5-trifluorophenyl)propanenitrile (140 g, 384 mmol, 85% yield) as a yellow solid. Analysis condition E: Retention time=3.78 min; ESI-MS(+) m/z [M+H]⁺: 365.2.

### Step 2

To a stirred solution of 2-((diphenylmethylene)amino)-3-(3,4,5-trifluorophenyl)propanenitrile (80 g, 220 mmol) in 1,4-dioxane (240 mL), was added conc. HCl (270 mL, 3293 mmol) and the mixture was stirred at 90° C. for 16 h. The reaction mixture was taken as such for the next step.

### Step 3

To the crude aqueous dioxane solution from the previous was added 10 N NaOH solution until the solution was neutral. Na₂CO₃ (438 mL, 438 mmol) was then added, followed by the addition of Fmoc-OSu (81 g, 241 mmol). The mixture was stirred at RT overnight. The aqueous solution was acidified with 1.5 N HCl to pH=2 and the solid formed was filtered, and dried to afford the crude compound. It was slurried initially with 5% EtOAc/petroleum ether for 30 min and filtered. The filtered compound was further slurried with ethyl acetate for 20 min and filtered to get the crude racemic 2-((((9H-fluoren-9-yl)methoxy)carbonyl)amino)-3-(3,4,5-trifluorophenyl)propanoic acid (90 g, 204 mmol, 93% yield) as an off-white solid. This racemic compound was separated into two isomers by SFC purification to get the desired isomers. After concentration of the desired isomer, it was slurried with 5% EtOAc/petroleum ether and filtered to get (S)-2-((((9H-fluoren-9-yl)methoxy)carbonyl)amino)-3-(3,4,5-trifluorophenyl)propanoic acid (43 g, 95 mmol, 43.3% yield) as an off-white solid. ¹H NMR (MeOD, 400 MHz) δ 7.78 (d, J=7.2 Hz, 2H), 7.60 (t, J=8.0 Hz, 2H), 7.38 (t, J=8.0 Hz, 2H), 7.28 (t, J=7.6 Hz, 2H), 7.01 (t, J=7.8 Hz, 2H), 4.48-4.26 (m, 3H), 4.18 (m, 1H), 3.18 (m, 1H), 2.91 (m, 1H). ¹⁹F (MeOD, 376 MHz) δ −137.56 (d, J=19.6 Hz, 2F), −166.67 (t, J=19.6 Hz, 1F). Analysis condition E: Retention time=3.15 min; ESI-MS(+) m/z [M+H]⁺: 442.2.

The other fraction was concentrated to get (R)-2-((((9H-fluoren-9-yl)methoxy)carbonyl)amino)-3-(3,4,5-trifluorophenyl)propanoic acid (40 g, 91 mmol, 41.4% yield) as an off-white solid.

### Preparation of (S)-2-((((9H-fluoren-9-yl)methoxy)carbonyl)amino)-4-(tert-butoxy)-3,3-dimethyl-4-oxobutanoic acid

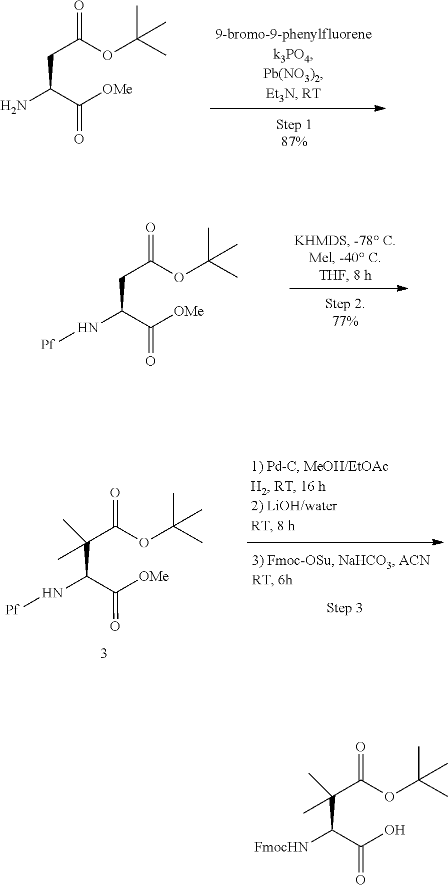

### Step 3

To a stirred solution of 4-(tert-butyl) 1-methyl L-aspartate, HCl salt (34 g, 142 mmol) in acetonitrile (550 mL), was added lead(II) nitrate (47.0 g, 142 mmol), potassium phosphate (66.2 g, 312 mmol), and TEA (19.77 mL, 142 mmol) under nitrogen atmosphere. The mixture was cooled to 0° C. then a solution of 9-bromo-9-phenylfluorene (43.3 g, 135 mmol) in acetonitrile (100 mL) was added. The reaction mixture was stirred at RT for 48 h and the reaction progress was monitored by TLC (50% Ethyl Acetate in Pet Ether) and LCMS. The reaction mixture was filtered over celite, washed with chloroform, and evaporated to get thick pale yellow liquid, to which ethyl acetate (3500 mL) was added. The EtOAc layer was washed with 5% citric acid solution (500 mL) followed by brine solution. The organic layer was dried over sodium sulfate and evaporated under reduced pressure to get pale yellow thick liquid, which was scratched with petroleum ether and filtered to obtain 4-(tert-butyl) 1-methyl (9-phenyl-9H-fluoren-9-yl)-L-aspartate (55 g, 124 mmol, 87% yield) as a white solid. Analysis condition L: Retention time=1.73 min; ESI-MS(+) m/z [M+Na]⁺: 466.40.

### Step 2

A solution of 4-(tert-butyl) 1-methyl (9-phenyl-9H-fluoren-9-yl)-L-aspartate (22.5 g, 50.7 mmol) was cooled to −78° C. under Ar and a solution of KHMDS (127 mL, 127 mmol, 1 M in THF) was added over 30 min while stirring. The reaction was allowed to warm to −40° C., and methyl iodide (9.52 mL, 152 mmol) was added dropwise. The reaction was stirred at −40° C. for 5 h. The reaction was monitored by TLC and LCMS. Saturated NH₄Cl (400 mL) was added followed by H₂O (100 mL). The resulting mixture was extracted with EtOAc (3×) and the combined organic extracts were washed with 2% citric acid (200 mL), aq. NaHCO₃ (200 mL), and brine. The organic layer was dried over anhydrous Na₂SO₄, evaporated in vacuo, and recrystallized from hexanes to give 1-(tert-butyl) 4-methyl (S)-2,2-dimethyl-3-((9-phenyl-9H-fluoren-9-yl)amino)succinate (18.5 g, 39.2 mmol, 77% yield) as a white solid, which was taken for next the step. Analysis condition L: Retention time=2.04 min; ESI-MS(+) m/z [M+Na]: 494.34.

### Step 3

A stirred solution of 1-(tert-butyl) 4-methyl (S)-2,2-dimethyl-3-((9-phenyl-9H-fluoren-9-yl)amino)succinate (24 g, 50.9 mmol) in methanol (270 mL) and ethyl acetate (100 mL) was degassed with nitrogen. Pd—C (2.71 g, 2.54 mmol) (10% by weight) was added, and the mixture was flushed with hydrogen gas and then stirred at RT in a 1-liter capacity autoclave with 50 psi overnight. The reaction mixture was filtered through celite pad, washed with a mixture of methanol and ethyl acetate. The combined solvents were evaporated to dryness and the precipitated white solid was removed by filtration to obtain a pale yellow liquid 1-(tert-butyl) 4-methyl (S)-3-amino-2,2-dimethylsuccinate (11.7 g) which was taken as such for the next step.

### Step 4

To a stirred solution of 1-(tert-butyl) 4-methyl (S)-3-amino-2,2-dimethylsuccinate (11.0 g, 47.6 mmol), cooled in an ice bath, was added lithium hydroxide (428 mL, 86 mmol, 0.2 M solution in water) and the reaction was slowly brought to RT. The reaction was monitored by TLC and LCMS. The reaction mixture was evaporated and directly taken to the next step. To a stirred solution of (S)-2-amino-4-(tert-butoxy)-3,3-dimethyl-4-oxobutanoic acid (15 g, 69.0 mmol) (which was in water from the previous batch) in acetonitrile (200 mL) cooled to 0° C., was added sodium bicarbonate (5.80 g, 69.0 mmol) and Fmoc-OSu (46.6 g, 138 mmol). The reaction mixture was stirred at RT overnight. It was acidified with 2 N HCl to pH=4, then extracted with ethyl acetate (3×500 mL), and the combined organic layer was washed with brine, dried over sodium sulfate, and evaporated to get an off-white solid, which was purified by ISCO flash chromatography with 20% Ethyl Acetate in petroleum ether to get (S)-2-((((9H-fluoren-9-yl)methoxy)carbonyl)amino)-4-(tert-butoxy)-3,3-dimethyl-4-oxobutanoic acid (12.2 g, 26.9 mmol, 39.0% yield) as a white solid. ¹HNMR (CDCl₃, 400 MHz) δ 7.77 (d, J=7.6 Hz, 2H), 7.60 (m, 2H), 7.42 (t, J=8.0 Hz, 2H), 7.33 (t, J=7.6 Hz, 2H), 4.65 (m, 2H), 4.34 (m, 1H), 4.25 (m, 1H), 3.18 (m, 1H), 1.40-1.27 (m, 6H). Analysis condition E: Retention time=1.90 min; ESI-MS(+) m/z [M+H]⁺: 440.2.

### Preparation of(S)-2-((((9H-fluoren-9-yl)methoxy)carbonyl)amino)-3-(3-(tert-butoxycarbonyl)phenyl)propanoic acid

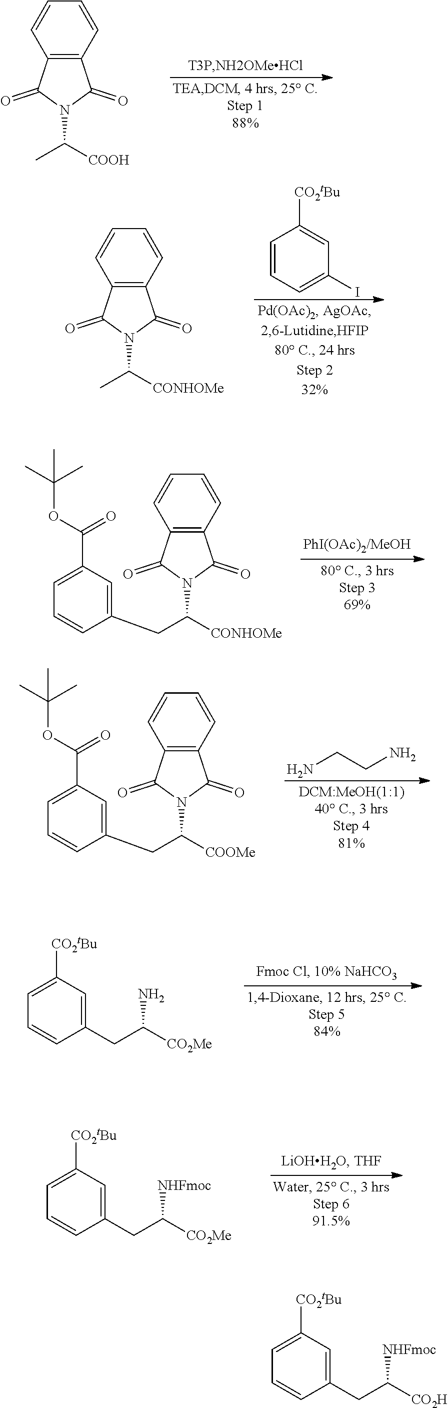

### Step 1

To a solution of (S)-2-(1,3-dioxoisoindolin-2-yl)propanoic acid (80 g, 365 mmol), O-methylhydroxylamine hydrochloride (36.6 g, 438 mmol) in CH₂Cl₂ (2000 mL), was added TEA (153 mL, 1095 mmol) at RT. The reaction was cooled to 0° C., 1-propanephosphonic anhydride (326 mL, 547 mmol) was added dropwise. The reaction was stirred at RT for 2 h. It was quenched with saturated ammonium chloride (500 mL) and extracted with EtOAc (3×300 mL). The combined organic layers were washed with saturated brine, dried over Na₂SO₄, and concentrated under reduced pressure. The crude product was purified via combiflash using 120 g silica column with 38 to 45% EtOAc in petroleum ether to give (S)-2-(1,3-dioxoisoindolin-2-yl)-N-methoxypropanamide (80 g, 322 mmol, 88% yield). ¹H NMR (DMSO-d₆, 400 MHz) δ 11.36 (s, 1H), 7.91-7.85 (m, 4H), 4.75-4.69 (m, 1H), 3.56 (s, 3H), 1.51 (d, J=7.6 Hz, 3H).

### Step 2

To a solution of (S)-2-(1,3-dioxoisoindolin-2-yl)-N-methoxypropanamide (20 g, 81 mmol), palladium(II) acetate (1.809 g, 8.06 mmol), silver acetate (26.9 g, 161 mmol) placed in a 1000-mL seal tube, was added tert-butyl 3-iodobenzoate (36.8 g, 121 mmol), 2,6-Lutidine (2.395 mL, 24.17 mmol), HFIP (300 mL) at 25° C. under N₂ atmosphere. The reaction was stirred for 15 min at 25° C. under N₂ and then heated up to 80° C. for 24 h with vigorous stirring. The reaction mixture was filtered through celite and washed with DCM (200 mL). The combined organic layer was concentrated under reduced pressure. The crude product was purified via combiflash using 220 g silica column eluting with 25 to 30% EtOAc:CHCl₃ to obtain the desired product tert-butyl (S)-3-(2-(1,3-dioxoisoindolin-2-yl)-3-(methoxyamino)-3-oxopropyl)benzoate (11 g, 25.9 mmol, 32.2% yield). Analysis condition E: Retention time=2.52 min; ESI-MS(+) m/z [M−H]⁺: 423.2. ¹H NMR (DMSO-d₆, 400 MHz) δ 11.46 (s, 1H), 7.82 (m, 4H), 7.63 (d, J=7.6 Hz, 1H), 7.54 (s, 1H), 7.40 (d, J=7.6 Hz, 1H), 7.30 (t, J=7.6 Hz, 1H), 4.93-4.89 (m, 1H), 3.59 (s, 3H), 3.56-3.49 (m, 1H), 3.36-3.27 (m, 1H), 1.40 (s, 9H).

### Step 3

To a solution of tert-butyl (S)-3-(2-(1,3-dioxoisoindolin-2-yl)-3-(methoxyamino)-3-oxopropyl)benzoate (15 g, 35.3 mmol) in methanol (200 mL), (diacetoxyiodo)benzene (12.52 g, 38.9 mmol) was added at RT. The temperature was slowly raised to 80° C. and stirred for 3 h at 80° C. The Reaction was concentrated under reduced pressure to get the crude product. It was purified with silica gel chromatography (100-200 mesh eluting with 20% Ethyl Acetate: hexane) to obtain the desired compound tert-butyl (S)-3-(2-(1,3-dioxoisoindolin-2-yl)-3-methoxy-3-oxopropyl)benzoate (10 g, 24.42 mmol, 69.1% yield. ¹H NMR (CDCl₃, 400 MHz) δ 7.80-7.76 (m, 4H), 7.72-7.68 (m, 2H), 7.34-7.26 (m, 1H), 7.25-7.23 (m, 1H), 5.14 (dd, J=10.8, 5.6 Hz, 1H), 3.76 (s, 3H), 3.65-3.49 (m, 2H), 1.50 (s, 9H).

### Step 4

To a solution of tert-butyl (S)-3-(2-(1,3-dioxoisoindolin-2-yl)-3-methoxy-3-oxopropyl)benzoate (15 g, 36.6 mmol) in methanol (25 mL) ethylenediamine (12.25 mL, 183 mmol) was added at RT. The reaction temperature was slowly raised to 40° C. and stirred for 3 h at 40° C. The mixture was concentrated under reduced pressure to get the crude product. It was purified with silica gel chromatography (100-200 mesh eluting with 20% Ethyl Acetate: hexane) to obtain the desired compound tert-butyl (S)-3-(2-amino-3-methoxy-3-oxopropyl)benzoate (8.3 g, 29.7 mmol, 81% yield). ¹H NMR (DMSO-d₆, 400 MHz) δ 8.32 (s, 1H), 7.77-7.72 (m, 2H), 7.46-7.38 (m, 1H), 3.61-3.57 (m, 4H), 2.96-2.91 (m, 1H), 2.85-2.82 (m, 1H), 1.79 (br. s, 2H), 1.55 (s, 9H).

### Step 5

To a solution of tert-butyl (S)-3-(2-amino-3-methoxy-3-oxopropyl)benzoate (10 g, 35.8 mmol) in dioxane (150 mL), sodium bicarbonate (6.01 g, 71.6 mmol) was added followed by the addition of 9-fluorenylmethyl chloroformate (13.89 g, 53.7 mmol) at RT. The reaction was stirred for 12 h at RT. It was diluted with water and extracted with ethyl acetylate. The organic layer was concentrated under reduced pressure to get the crude product. It was purified via silica gel chromatography (100-200 mesh eluting with 20% Ethyl Acetate: hexane) to obtain the desired compound tert-butyl (S)-3-(2-((((9H-fluoren-9-yl)methoxy)carbonyl)amino)-3-methoxy-3-oxopropyl)benzoate (15 g, 29.9 mmol, 84% yield).

### Step 6

To a solution of tert-butyl (S)-3-(2-((((9H-fluoren-9-yl)methoxy)carbonyl)amino)-3-methoxy-3-oxopropyl)benzoate (18.00 g, 35.9 mmol) in THF (150 mL) and H₂O (150 mL) at RT, lithium hydroxide monohydrate (1.66 g, 39.5 mmol) was added. The reaction was stirred for 2 h at RT. The reaction was concentrated under reduced pressure to remove THF. In the basic medium the mixture was extracted with diethyl ether to remove the non polar impurities. The aqueous layer was acidified with aqueous citric acid solution and extracted with ethyl acetate. The organic layer was dried over sodium sulphate and concentrated under reduced to get the desired compound as a gummy solid which was further lyopholized to provide white solids of the desired compound Lot 1: (S)-2-((((9H-fluoren-9-yl)methoxy)carbonyl)amino)-3-(3-(tert-butoxycarbonyl)phenyl)propanoic acid (11 g, 22.56 mmol, 62.9% yield) And lot 2: (S)-2-((((9H-fluoren-9-yl)methoxy)carbonyl)amino)-3-(3-(tert-butoxycarbonyl)phenyl)propanoic acid (5 g, 10.26 mmol, 28.6% yield). 7.86 (t, J=7.6 Hz, 2H), 7.75 (d, J=7.6 Hz, 1H), 7.66-7.59 (m, 2H), 7.52 (m, 2H), 7.41-7.37 (m, 3H), 7.31-7.24 (m, 2H), 4.21-4.16 (m, 4H), 3.17 (m, 1H), 2.96 (m, 1H), 1.53 (br, s. 9H). Analysis condition E: Retention time=3.865 min; ESI-MS(+) m/z [M−H]⁺: 486.2.

### Preparation of (S)-2-((((9H-fluoren-9-yl)methoxy)carbonyl)amino)-3-(m-tolyl)propanoic acid

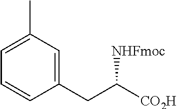

Compound was synthesized following the similar procedures of (S)-2-((((9H-fluoren-9-yl)methoxy)carbonyl)amino)-3-(3-(tert-butoxycarbonyl)phenyl)propanoic acid. Analysis condition E: Retention time=3.147 min; ESI-MS(+) m/z [M+H]⁺: 402.0. ¹H NMR (DMSO-d₆, 300 MHz) δ 7.88 (d, J=7.5 Hz, 2H), 7.64 (t, J=6.8 Hz, 2H), 7.44 (t, J=7.5 Hz, 2H), 7.36-7.28 (m, 2H), 7.18 (t, J=7.5 Hz, 1H), 7.09-7.02 (m, 3H), 4.24-4.17 (m, 4H), 3.21-3.04 (m, 1H), 2.89-2.81 (m, 1H), 2.26 (s, 3H) ppm.

### Preparation ethyl (S)-5-((tert-butoxycarbonyl)amino)-2-(((S)-mesitylsulfnyl)amino)-3,3-dimethylpentanoate

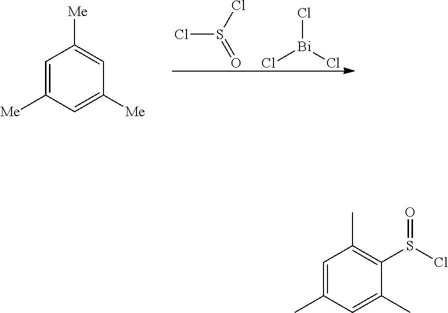

### Step 1

To a 1000-mL flask equipped with a septum inlet and magnetic stirring bar was added bismuth(III) chloride (5.25 g, 16.64 mmol). The flask was connected to an argon line and thionyl chloride (501 mL, 6864 mmol) were added by syringe. To the suspension was added mesitylene (100 g, 832 mmol). The flask was equipped with a condenser, connected to an oil bubbler and the reaction mixture was heated in an oil bath at 60° C. for 5 h. During this time the color of the solution became red-orange and HCl evolved from the solution. The reaction was monitored by LCMS. The flask was cooled in an ice bath and the excess of thionyl chloride was removed under reduced pressure yielding an orange liquid. In order to remove the catalyst, 2000 mL of pentane were added, stirred and filtered through celite, and the bed was washed with pentane (2×500 mL). The organic phase was collected and evaporated under reduced pressure to give 2,4,6-trimethylbenzenesulfinic chloride (151 g, 745 mmol, 90% yield) as a pale yellow solid. The compound was taken to the next step without further purification. ¹H NMR (400 MHz, CDCl₃) δ 7.07-6.76 (m, 2H), 2.66 (s, 6H), 2.38-2.24 (m, 3H) ppm.

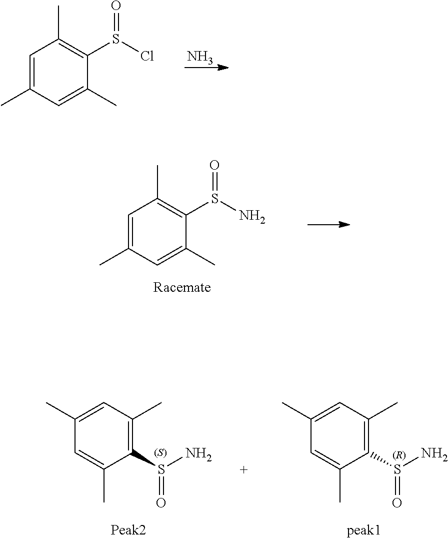

### Step 2

A stirred solution of 2,4,6-trimethylbenzenesulfinic chloride (155 g, 765 mmol) in diethyl ether (1500 mL) was cooled to −40° C. In a separate setup, (2 L multi neck RBF) taken in diethyl ether (900 mL) ammonia gas was bubbled 30 minutes at −40° C. This purged solution was added to the above reaction mass at −40° C. After it had warmed to RT the reaction mixture was stirred for 2 hours and monitored by open access LCMS until starting material was absent. The reaction was stirred at room temperature overnight according to given procedure. The reaction was monitored by TLC and open access LCMS, TLC wise starting material was absent. Workup: The reaction mixture was diluted with ethyl acetate (3000 mL) and washed with water(2000 mL). The organic layer was separated and the aqueous phase was again extracted with ethyl acetate (lx 500 mL). The combined organic layer washed with brine(lx 800 mL). The combined organic layer, dried (Na₂SO₄), filtered, and concentrated under reduced pressure to obtained (235 g) as a pale brown solid. The product (235 g) was recrystallized from 10% ethyl acetate/petroleum ether (500 mL), stirred, filtered, and dried to afford mesitylenesulphinamide (125 g) racemate as a white solid. The compound was submitted for the SFC method development. Two peaks were collected from SFC. The solvent was concentrated to give Peak-1 (Undesired): (R)-2,4,6-trimethylbenzenesulfinamide (51.6 g, 265 mmol, 34.6% yield) as a white colour solid. ¹H NMR (400 MHz, DMSO-d₆) δ 7.01-6.68 (m, 2H), 6.23-5.77 (m, 2H), 2.52-2.50 (m, 6H), 2.32-1.93 (m, 3H) and Peak-2 (desired): (S)-2,4,6-trimethylbenzenesulfinamide (51.6 g, 267 mmol, 35.0% yield) as a white colour solid. ¹H NMR (400 MHz, DMSO-d₆) δ 6.87 (s, 2H), 6.16-5.82 (m, 2H), 2.53-2.50 (m, 6H), 2.34-1.93 (m, 3H).

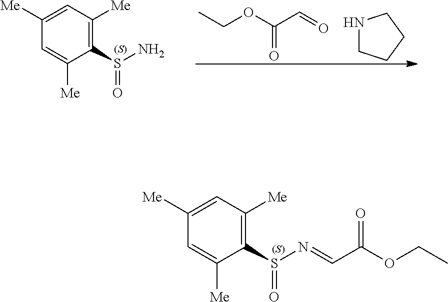

### Step 3

To a well stirred solution of (S)-2,4,6-trimethylbenzenesulfinamide (15.5 g, 85 mmol) in dichloromethane (235 mL) and 4 A molecular sieves (84.5 g), was added ethyl 2-oxoacetate in toluene (25.9 mL, 127 mmol) and pyrrolidine (0.699 mL, 8.46 mmol). The reaction mixture was stirred at room temperature for overnight. The reaction was repeated and the two batches were combined together for work up. The reaction mass was filtered through celite and the bed was washed with DCM. The solvents were removed under reduced pressure to obtain the crude (55 g) as a brownish color mass. The crude compound was purified by ISCO (Column size: 300 g silica column. Adsorbent: 60-120 silica mesh, Mobile phase:40% EtOAc/Pet ether) and the product was collected at 15-20% of EtOAc. The fractions were concentrated to obtain ethyl (S,E)-2-((mesitylsulfnyl)imino)acetate (16.5 g, 57.4 mmol, 67.9% yield) as a colorless liquid. The compound slowly solidified as an off white solid. ¹H NMR (400 MHz, CDCl₃) δ=8.27 (s, 1H), 7.04-6.70 (m, 2H), 4.59-4.21 (m, 2H), 2.55-2.44 (m, 6H), 2.36-2.23 (m, 3H), 1.51-1.30 (m, 3H). 2.670 min. 268.2 (M+H).

### Step 4

General procedure for the synthesis of TCNHPI redox-active esters: A round-bottom flask or culture tube was charged with carboxylic acid (1.0 equiv), N-hydroxytetrachlorophthalimide (1.0-1.1 equiv) and DMAP (0.1 equiv). Dichloromethane was added (0.1-0.2 M), and the mixture was stirred vigorously. Carboxylic acid (1.0 equiv) was added. DIC (1.1 equiv) was then added dropwise via syringe, and the mixture was allowed to stir until the acid was consumed (determined by TLC). Typical reaction times were between 0.5 h and 12 h. The mixture was filtered (through a thin pad of diatomaceous earth (Celite®), SiO₂, or frit funnel) and washed with additional CH₂Cl₂/Et₂O. The solvent was removed under reduced pressure, and purification of the crude mixture by column chromatography afforded the desired TCNHPI redox-active ester. If necessary, the TCNHPI redox-active ester could be further recrystallized from CH₂C₂/MeOH.

### Step 5

4,5,6,7-tetrachloro-1,3-dioxoisoindolin-2-yl-4-((tert-butoxycarbonyl)amino)-2,2-dimethylbutanoate was obtained as a white solid following General Procedure for the synthesis of TCNHPI redox-active esters on 5.00 mmol scale. Purification by column (silica gel, gradient from CH₂Cl₂ to 10:1 CH₂Cl₂:Et₂O) afforded 2.15 g (84%) of the title compound. ¹H NMR (400 MHz, CDCl₃): 5 4.89 (br s, 1H), 3.30 (q, J=7.0 Hz, 2H), 1.98 (t, J=7.6 Hz, 2H), 1.42 (s, 15H) ppm. ¹³C NMR (151 MHz, CDCl₃): δ 173.1, 157.7, 156.0, 141.1, 130.5, 124.8, 79.3, 40.8, 40.2, 36.8, 28.5, 25.2 ppm. HRMS (ESI-TOF): calc'd for C₁₉H₂₀Cl₄N₂NaO₆ [M+Na]⁺: 534.9968, found: 534.9973.

### Step 6

Ethyl (S)-5-((tert-butoxycarbonyl)amino)-2-(((S)-mesitylsulfnyl)amino)-3,3-dimethylpentanoate was made using the General procedures for decarboxylative Amino acid synthesis in reference ACIE. A culture tube was charged with TCNHPI redox-active ester A (1.0 mmol), sulfinimine B (2.0 mmol), Ni(OAc)2·4H₂O (0.25 mmol, 25 mol %), Zinc (3 mmol, 3 equiv). The tube was then evacuated and backfilled with argon (three times). Anhydrous NMP (5.0 mL, 0.2 M) was added using a syringe. The mixture was stirred overnight at RT. Then, the reaction mixture was diluted with EtOAc, washed with water, brine and dried over MgSO₄. Upon filtration, the organic layer was concentrated under reduced pressure (water bath at 30° C.), and purified by flash column chromatography (silica gel) to provide the product. Purification by column (2:1 hexanes:EtOAc) afforded 327.6 mg (72%) of the title compound ethyl (S)-5-((tert-butoxycarbonyl)amino)-2-(((S)-mesitylsulfinyl)amino)-3,3-dimethylpentanoate as a colorless oil. ¹H NMR (600 MHz, CDCl₃): δ 6.86 (s, 2H), 5.04 (d, J=10.1 Hz, 1H), 4.47 (s, 1H), 4.28-4.16 (m, 2H), 3.66 (d, J=10.1 Hz, 1H), 3.27-3.05 (m, 2H), 2.56 (s, 6H), 2.28 (s, 3H), 1.54-1.46 (m, 2H), 1.43 (s, 9H), 1.30 (t, J=7.2 Hz, 3H), 0.96 (s, 6H) ppm. ¹³C NMR (151 MHz, CDCl₃): δ 172.5, 155.9, 141.1, 137.9, 136.9, 131.0, 79.4, 65.5, 61.7, 38.8, 37.1, 36.5, 28.5, 23.9, 23.6, 21.2, 19.4, 14.3 ppm. HRMS (ESI-TOF): calc'd for C₂₃H₃₉N₂O₅S [M+H]⁺: 455.2574, found: 455.2569.

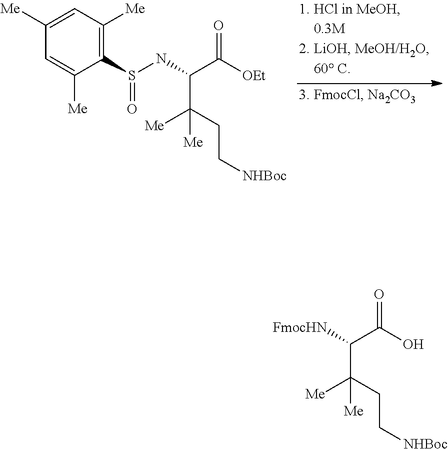

### Step 7

2-((((9H-fluoren-9-yl)methoxy)carbonyl)amino)-5-((tert-butoxycarbonyl)amino)-3,3-dimethylpentanoic acid: A culture tube was charged with ethyl (S)—S-((tert-butoxycarbonyl)amino)-2-(((S)-mesitylsulfinyl)amino)-3,3-dimethylpentanoate (0.5 mmol, 1.0 equiv), HCl (4.0 equiv) in MeOH (0.3 M) was added via syringe and the resulting mixture was stirred at RT for ca. 10 min (screened by TLC). After the reaction, Et₃N was added until pH=7 and the solvents were removed under reduced pressure. LiOH (2 equiv) in MeOH/H₂O (2:1, 0.04 M) was added to the crude mixture. The reaction was stirred at 60° C. overnight. On completion, HCl in MeOH (0.3 M) was added until pH=7 and the solvents were removed under reduced pressure. The crude mixture was dissolved in 9% aqueous Na₂CO₃ (5 mL) and dioxane (2 mL). It was slowly added at 0° C. to a solution of Fmoc-OSu (1.2 equiv) in dioxane (8 mL). The mixture was stirred at 0° C. for 1 h and then allowed to warm to RT. After 10 h, the reaction mixture was quenched with HCl (0.5 M), reaching pH 3, and then diluted with EtOAc. The aqueous phase was extracted with EtOAc (3×15 mL), and the combined organic layers were washed with brine, dried over Na₂SO₄, filtered, and the solvent was removed under reduced pressure. The crude mixture was then purified by flash column chromatography (silica gel, 2:1 hexanes:EtOAc) to afford the product ethyl (S)—S-((tert-butoxycarbonyl)amino)-2-(((S)-mesitylsulfnyl)amino)-3, 3-dimethylpentanoate in 68% overall yield and 95% ee as a colorless oil. ¹H NMR (600 MHz, CDCl₃): δ 7.76 (d, J=7.5 Hz, 2H), 7.63-7.54 (m, 2H), 7.39 (td, J=7.3, 2.6 Hz, 2H), 7.33-7.28 (m, 2H), 5.50 (br s, 1H), 4.68 (br s, 1H), 4.45-4.43 (m, 1H), 4.38-4.35 (m, 1H), 4.30 (d, J=7.9 Hz, 1H), 4.21 (t, J=6.8 Hz, 1H), 3.27 (br s, 1H), 3.16 (br s, 1H), 1.63-1.50 (m, 2H), 1.43 (s, 9H), 1.09-0.76 (m, 6H) ppm. ¹³C NMR (151 MHz, CDCl₃): δ 185.8, 174.3, 156.5, 144.0, 143.9, 141.5, 127.9, 127.2, 125.24, 125.21, 120.2, 120.1, 79.8, 67.2, 60.9, 47.4, 39.2, 36.8, 29.9, 28.6, 23.9 ppm. HRMS (ESI-TOF): calc'd for C₂₇H₃₅N₂O₆ [M+H]⁺: 483.2490, found: 483.2489.

### Preparation of(S)-2-((((9H-fluoren-9-yl)methoxy)carbonyl)amino)-3-(4,4-difluorocyclohexyl)propanoic acid

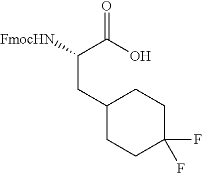

Final product was obtained following similar procedures of ethyl (S)—S-((tert-butoxycarbonyl)amino)-2-(((S)-mesitylsulfinyl)amino)-3,3-dimethylpentanoate. The synthesis afforded the desired (S)-2-((((9H-fluoren-9-yl)methoxy)carbonyl)amino)-3-(4,4-difluorocyclohexyl)propanoic acid (60 mg, 0.14 mmol, 27.9% yield) as a white solid after purification by reverse phase HPLC. ¹H NMR (500 MHz, CDCl₃) δ 7.79 (br d, J=7.5 Hz, 2H), 7.61 (br s, 2H), 7.43 (s, 2H), 7.36-7.31 (m, 2H), 5.24-5.06 (m, 1H), 4.57-4.36 (m, 3H), 4.29-4.16 (m, 1H), 2.19-1.99 (m, 2H), 1.97-1.18 (m, 9H).

### Preparation of (2S)—S-(tert-butoxy)-2-({[(9H-fluoren-9-yl)methoxy]carbonyl}amino)-3,3-dimethyl-5-oxopentanoic acid

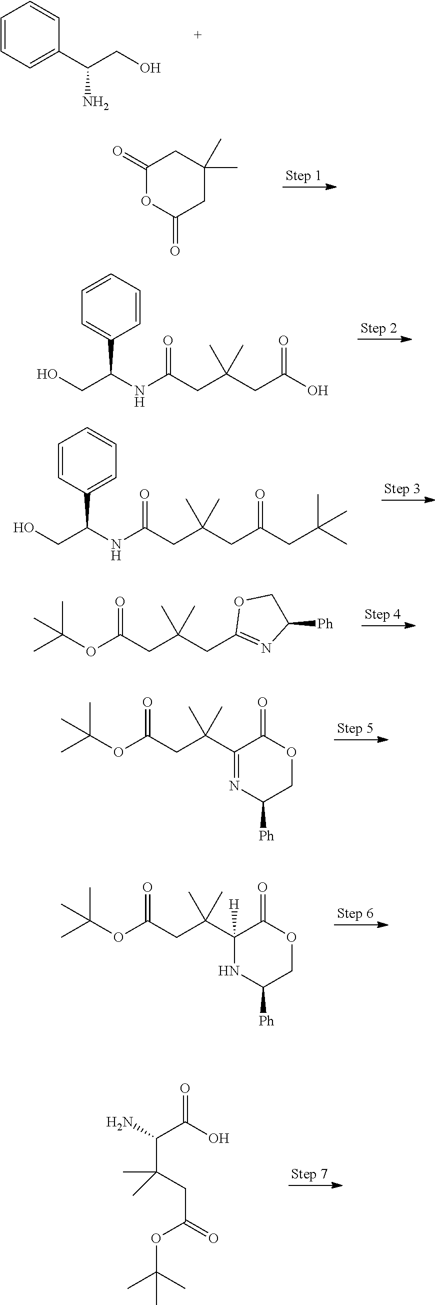

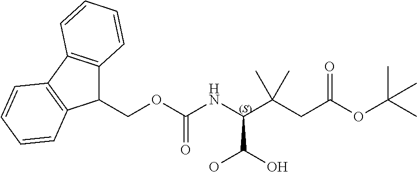

### Step 1

A solution of 4,4-dimethyldihydro-2H-pyran-2,6(3H)-dione (8.29 g, 58.3 mmol) in dry toluene (100 mL) was slowly added to a solution of (R)-2-amino-2-phenylethan-1-ol (10 g, 72.9 mmol) in dry toluene (100 mL) and CH₂Cl₂ (20 mL) at room temperature. The reaction mixture was then heated to 60° C. and reacted for 12 h. It was cooled to room temperature until a white solid was formed. The solid was filtered and washed with 1:1 EtOAc/CH₂Cl₂ to afford the crude desired compound (R)-5-((2-hydroxy-1-phenylethyl)amino)-3,3-dimethyl-5-oxopentanoic acid (11.9 g, 41.0 mmol, 56.2% yield) without further purification. ¹H NMR (300 MHz, DMSO-d₆) δ 8.41 (br d, J=7.9 Hz, 1H), 7.44-7.32 (m, 2H), 7.32-7.27 (m, 4H), 7.26-7.18 (m, 1H), 4.89-4.80 (m, 1H), 4.14-3.98 (m, 1H), 3.63-3.43 (m, 3H), 2.27-2.18 (m, 4H), 2.08 (s, 1H), 1.99 (s, 1H), 1.17 (t, J=7.2 Hz, 1H), 1.00 (d, J=4.5 Hz, 6H), 0.92 (s, 1H).

### Step 2

(R)-5-((2-Hydroxy-1-phenylethyl)amino)-3,3-dimethyl-5-oxopentanoic acid (12 g, 43.0 mmol) was dissolved in a solution of benzyltri methylammonium chloride (8.93 g, 48.1 mmol) in DMA (250 mL). K₂CO₃ (154 g, 1117 mmol) was added to the above solution followed by the addition of 2-bromo-2-methylpropane (235 mL, 2091 mmol). The reaction mixture was stirred at 55° C. for 24 h. The reaction mixture was then diluted with EtOAc (100 mL), washed with H₂O (50 mL×3), and brine (50 mL). The organic phase was dried over Na₂SO₄, concentrated under vacuo, and purified by flash column chromatography on silica gel (CH₂Cl₂MeOH, 15:1) to give tert-butyl (R)-5-((2-hydroxy-1-phenylethyl)amino)-3,3-dimethyl-5-oxopentanoate (6.0 g, 17.89 mmol, 41.6% yield). Analytical LCMS Condition M: 1.96 min, 336.3 [M+H]⁺. ¹H NMR (300 MHz, DMSO-d₆) d=8.14 (br d, J=8.3 Hz, 1H), 7.33-7.25 (m, 4H), 7.25-7.17 (m, 1H), 4.90-4.77 (m, 2H), 3.52 (br t, J=5.7 Hz, 2H), 3.34 (s, 1H), 2.94 (s, 1H), 2.78 (s, 1H), 2.20 (d, J=14.0 Hz, 4H), 1.97 (d, J=9.8 Hz, 2H), 1.41-1.31 (m, 9H), 1.00 (d, J=1.1 Hz, 6H).

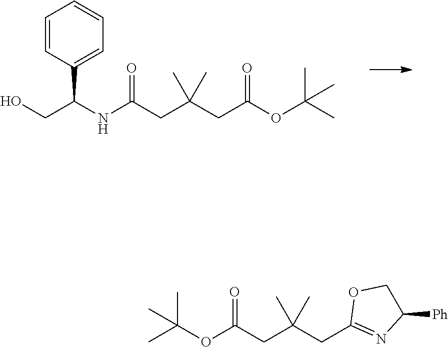

### Step 3

tert-Butyl (R)-5-((2-hydroxy-1-phenylethyl)amino)-3,3-dimethyl-5-oxopentanoate (6 g, 17.89 mmol) and 2,3-dichloro-5,6-dicyano-p-benzoquinone (6.09 g, 26.8 mmol) was dissolved in dry dichloromethane (70 mL) under Ar. Triphenylphosphine (7.04 g, 26.8 mmol) was added to the above solution. The reaction mixture was stirred at room temperature for 2 h. The crude product was then concentrated under vacuo and purified by flash column chromatography on silica gel (EtOAc/Hexanes, 1:5) to give tert-butyl (R)-3,3-dimethyl-4-(4-phenyl-4,5-dihydrooxazol-2-yl)butanoate (5.6 g, 17.64 mmol, 99% yield). ESI-MS(+) m/z: 318.3 [M+H]⁺. ¹H NMR (300 MHz, DMSO-d₆) d=7.41-7.18 (m, 5H), 5.18 (t, J=9.1 Hz, 1H), 4.59 (dd, J=8.7, 10.2 Hz, 1H), 3.94-3.85 (m, 1H), 3.94-3.85 (m, 1H), 3.95-3.84 (m, 1H), 4.10-3.84 (m, 1H), 2.43-2.22 (m, 4H), 1.40 (s, 9H), 1.09 (d, J=1.9 Hz, 6H).

### Step 4

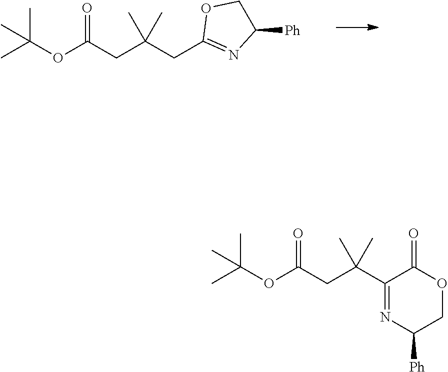 [SMILES: CC(C)(CC(=O)OC(C)(C)C)CC1=N[C@H](c2ccccc2)CO1 ; CC(C)(C)OC(=O)CC(C)(C)C1=N[C@H](c2ccccc2)COC1=O]

To a solution of tert-butyl (R)-3,3-dimethyl-4-(4-phenyl-4,5-dihydrooxazol-2-yl)butanoate (5.6 g, 17.64 mmol) in EtOAc (250 mL) was added selenium dioxide (4.89 g, 44.1 mmol) and refluxed for 2 h. The reaction mixture was then cooled to room temperature and stirred for 12 h. The crude product was then concentrated in vacuo and purified by flash column chromatography on silica gel (EtOAc/Hexanes, 1:7) to afford tert-butyl (R)-3-methyl-3-(2-oxo-5-phenyl-5,6-dihydro-2H-1,4-oxazin-3-yl)butanoate (1.3 g, 3.92 mmol, 22.23% yield) as a colorless liquid. ESI-MS(+) m/z: 332.2 [M+H]⁺. ¹H NMR (CDCl₃) δ 1.37 (s, 3H), 1.42 (s, 9H), 1.44 (s, 3H), 2.59 (d, J=15.5 Hz, 1H), 3.12 (d, J=15.5 Hz, 1H), 4.32 (t, J=11.1 Hz, 1H), 4.47 (dd, J=4.3 Hz, J=6.7 Hz, 1H), 4.80 (dd, J=4.3 Hz, J=6.7 Hz, 1H), 7.35-7.39 (m, 5H). ¹³C NMR (CD3Cl) δ 26.40, 27.29, 28.00, 40.84, 45.94, 59.72, 70.88, 80.63, 127.13, 127.92, 128.65, 137.58, 155.07, 167.46, 171.95.

### Step 5

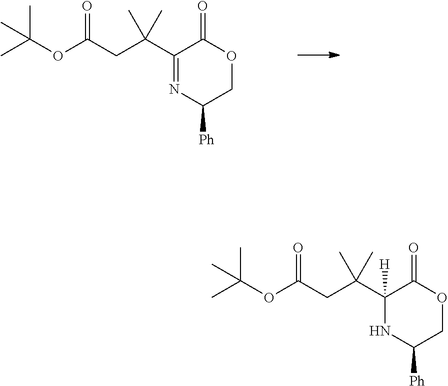 [SMILES: CC(C)(C)OC(=O)CC(C)(C)C1=N[C@H](c2ccccc2)COC1=O ; CC(C)(C)OC(=O)CC(C)(C)[C@@H]1N[C@H](c2ccccc2)COC1=O]

Platinum(IV) oxide monohydrate (130 mg, 0.530 mmol) was added to a solution of tert-butyl (R)-3-methyl-3-(2-oxo-5-phenyl-5,6-dihydro-2H-1,4-oxazin-3-yl)butanoate (1.3 g, 3.92 mmol) in methanol (50 mL). The reaction flask was purged with H₂ (3×) and stirred under H₂ for 24 h. After venting the vessel, the reaction mixture was filtered through diatomaceous earth (Celite®), and the filtrate was washed with EtOAc. The crude product was concentrated under vacuo and purified by flash column chromatography on silica gel (EtOAc/Hexanes, 1:8) to give tert-butyl 3-methyl-3-((3S,5R)-2-oxo-5-phenylmorpholin-3-yl)butanoate (1.2 g, 3.33 mmol, 85% yield). ¹H NMR (300 MHz, DMSO-d₆) δ 7.52-7.42 (m, 2H), 7.41-7.26 (m, 3H), 4.30-4.20 (m, 2H), 4.13 (d, J=10.6 Hz, 1H), 3.80 (d, J=7.6 Hz, 1H), 3.07-2.98 (m, 1H), 2.47 (br s, 1H), 2.27 (d, J=13.6 Hz, 1H), 1.43-1.35 (m, 9H), 1.17-1.07 (m, 5H).

### Step 6

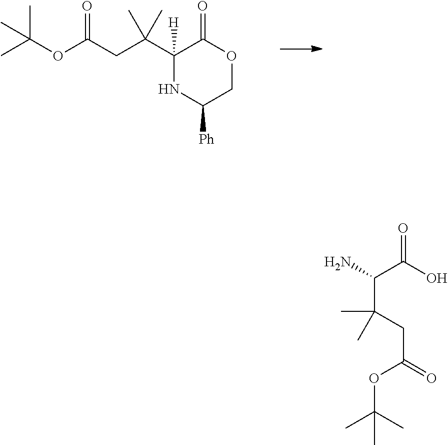 [SMILES: CC(C)(C)OC(=O)CC(C)(C)[C@@H]1N[C@H](c2ccccc2)COC1=O ; CC(C)(C)OC(=O)CC(C)(C)[C@H](N)C(=O)O]

Pearlman's catalyst Pd(OH)₂ on carbon (1.264 g, 1.799 mmol, 20% w/w) was added to a solution of tert-butyl 3-methyl-3-((3S,5R)-2-oxo-5-phenylmorpholin-3-yl)butanoate (1.2 g, 3.60 mmol) in methanol (50 mL)/water (3.13 mL)/TFA (0.625 mL) (40:2.5:0.5, v/v/v). The vessel was purged with H₂ and stirred under H₂ for 24 h. After venting the vessel, the reaction mixture was filtered through diatomaceous earth (Celite®), and the filtrate was washed with MeOH. The crude product ((S)-2-amino-5-(tert-butoxy)-3,3-dimethyl-5-oxopentanoic acid (0.83 g, 3.59 mmol, 100% yield)) was concentrated under vacuo. This crude was taken for the next step without further purification. Analytical LC/MS Condition M: 1.13 min, 232.2 [M+H]⁺.

### Step 7

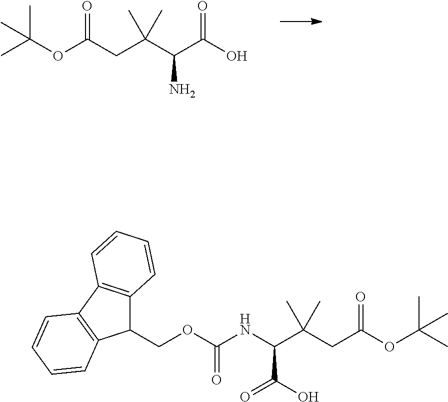 [SMILES: CC(C)(C)OC(=O)CC(C)(C)[C@H](N)C(=O)O ; CC(C)(C)OC(=O)CC(C)(C)[C@H](NC(=O)OCC1c2ccccc2-c2ccccc21)C(=O)O]

The crude product (S)-2-amino-5-(tert-butoxy)-3,3-dimethyl-5-oxopentanoic acid (1 g, 4.32 mmol) was dissolved in water (30 mL). Na₂CO₃ (0.916 g, 8.65 mmol) was then added to the above solution. To this solution, FMOC n-hydroxysuccinimide ester (1.458 g, 4.32 mmol) in dioxane (30 mL) was added dropwise at 0° C. and stirred at room temperature for 16 h. The reaction mixture was acidified to pH ˜2 by 1N HCl and extracted with EtOAc (50 mL×3), dried over Na₂SO₄, concentrated under vacuo and purified by flash column chromatography on silica gel (EtOAc/petroleum ether, 35 to 39%) to give (S)-2-((((9H-fluoren-9-yl)methoxy)carbonyl)amino)-5-(tert-butoxy)-3,3-dimethyl-5-oxopentanoic acid (0.73 g, 1.567 mmol, 36.2% yield) as a white solid. LCMS, Analytical LC MS Condition E. MS (ESI) t_{R}=2.135 min, m/z 452.2 [M−H]⁻. ¹H NMR (400 MHz, DMSO-d₆) δ 12.78-12.64 (m, 1H), 7.90 (d, J=7.5 Hz, 2H), 7.77 (dd, J=4.5, 7.0 Hz, 2H), 7.65 (br d, J=9.5 Hz, 1H), 7.46-7.39 (m, 2H), 7.37-7.29 (m, 2H), 4.32-4.15 (m, 4H), 2.39-2.31 (m, 1H), 2.30-2.21 (m, 1H), 1.39 (s, 9H), 1.12-1.00 (m, 6H).

### Preparation of (2S)-2-({[(9H-fluoren-9-yl)methoxy]carbonyl}amino)-3-(morpholin-4-yl)propanoic acid

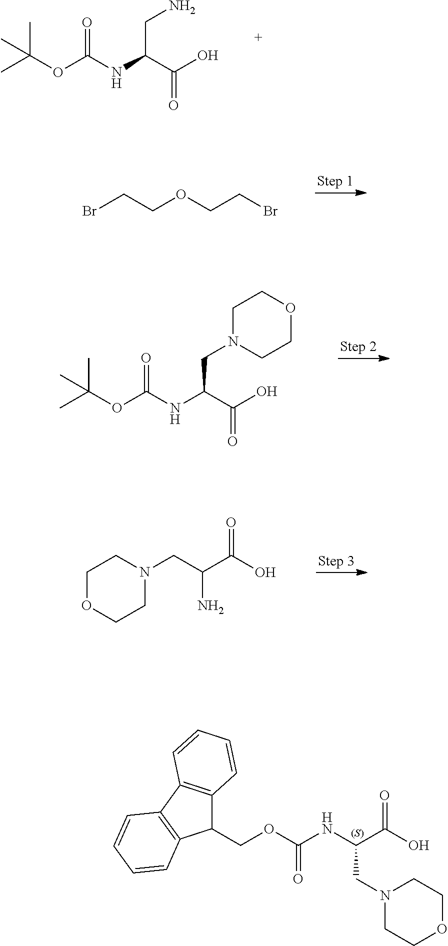

### Step 1

In a 2-L multi-necked round-bottomed flask fitted with a thermo pocket was added (S)-3-amino-2-((tert-butoxycarbonyl)amino)propanoic acid (50 g, 245 mmol), dioxane (500 mL), followed by 1-bromo-2-(2-bromoethoxy)ethane (30.8 mL, 245 mmol) at RT. NaOH (367 mL, 734 mmol) solution was added and the resulting yellow clear solution was heated to 110° C. (external temperature, 85° C. internal temperature) for 12 h. An aliquot of clear solution was subjected to LCMS (Polar method) which showed completion, and then the dioxane was evaporated to provide a light red solution which was acidified to pH 3. The resulting mixture was concentrated under high vacuum pump (˜4 mbar) at 60° C. to get (S)-2-((tert-butoxycarbonyl)amino)-3-morpholinopropanoic acid (67 g, 244 mmol, 100% yield) pale yellow solid. Analytical LC MS Condition M. 0.56 min, 275.2 [M+H]⁺.

### Step 2

To a stirred suspension of (S)-2-((tert-butoxycarbonyl)amino)-3-morpholinopropanoic acid (100 g, 365 mmol) in dioxane (400 mL) at 0-5° C. was added HCl in dioxane (911 mL, 3645 mmol) slowly over 20 min. The resulting mixture was stirred at RT for 12 h. The volatile was evaporated to get pale yellow sticky crude (S)-2-amino-3-morpholinopropanoic acid (16 g, 92 mmol, 97% yield), This crude was taken for next step without further purification. MS (ESI) m/z 175.2 [M+H]⁺.

### Step 3

The crude product (S)-2-amino-3-morpholinopropanoic acid (11 g, 63.1 mmol) was dissolved in water (250 mL), and Na₂CO₃ (13.39 g, 126 mmol) was then added to the above solution. To this solution, Fmoc N-hydroxysuccinimide ester (21.30 g, 63.1 mmol) was added dropwise at 0 C and stirred at room temperature for 16 h. The reaction mixture was acidified to pH ˜2 by 1N HCl and extracted with EtOAc (500 mL×3), dried over Na₂SO₄, concentrated under vacuo, and purified by flash column chromatography on silica gel (petroleum ether/EtOAc, 0-100% then MeOH/CHCl₃ 0-15%) to get (S)-2-((((9H-fluoren-9-yl)methoxy)carbonyl)amino)-3-morpholinopropanoic acid (23 g, 55.9 mmol, 89% yield) as a brown solid. Analytical LCMS Condition E: 1.43 min, 397.2 [M+H]⁺. ¹H NMR (400 MHz, METHANOL-d₄) δ 7.78 (br d, J=7.5 Hz, 2H), 7.71-7.57 (m, 2H), 7.42-7.34 (m, 2H), 7.34-7.26 (m, 2H), 4.71 (br s, 1H), 4.54-4.32 (m, 2H), 4.29-4.17 (m, 1H), 3.90 (br s, 4H), 3.76-3.62 (m, 1H), 3.58-3.47 (m, 1H), 3.41 (br s, 2H), 3.36-3.32 (m, 2H), 3.31-3.26 (m, 1H).

### Preparation of (2S,3S)-3-{[(tert-butoxy)carbonyl]amino}-2-({[(9H-fluoren-9-yl)methoxy]carbonyl}amino)butanoic acid

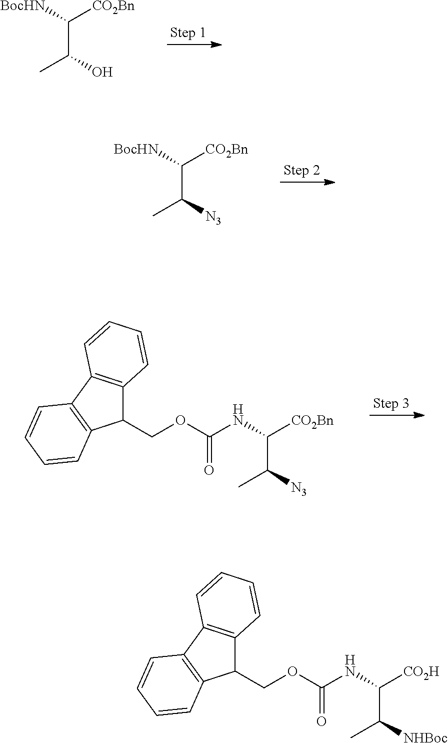 [SMILES: C[C@@H](O)[C@H](NC(=O)OC(C)(C)C)C(=O)OCc1ccccc1 ; C[C@H](N=[N+]=[N-])[C@H](NC(=O)OC(C)(C)C)C(=O)OCc1ccccc1 ; C[C@H](N=[N+]=[N-])[C@H](NC(=O)OCC1c2ccccc2-c2ccccc21)C(=O)OCc1ccccc1 ; C[C@H](NC(=O)OC(C)(C)C)[C@H](NC(=O)OCC1c2ccccc2-c2ccccc21)C(=O)O]

### Step 1

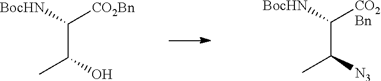 [SMILES: C[C@@H](O)[C@H](NC(=O)OC(C)(C)C)C(=O)OCc1ccccc1 ; C[C@H](N=[N+]=[N-])[C@H](NC(=O)OC(C)(C)C)C(=O)OCc1ccccc1]

To a solution of the benzyl (tert-butoxycarbonyl)-L-threoninate (22 g, 71.1 mmol) in CH₂Cl₂ (600 mL) at −78° C. was sequentially added trifluoromethanesulfonic anhydride (24.08 g, 85 mmol) dropwise and then 2,6-lutidine (10.77 mL, 92 mmol) slowly. After stirring at the same temperature for 1.5 h and monitoring by TLC (Hex:EtOAc 8:2), tetrabutylammonium azide (50.6 g, 178 mmol) was added in portions. After stirring at −78° C. for 1 h, the cooling bath was removed and the reaction mixture was allowed to reach 23° C. for 1.5 h. The reaction was repeated. A saturated aqueous solution of NaHCO₃ was added, and the aqueous phase extracted with EtOAc. The crude product was purified by flash chromatography over silica gel (Hex:EtOAc 95:5 a 9:1) to give benzyl (2S,3S)-3-azido-2-((tert-butoxycarbonyl)amino)butanoate (20 g, 59.8 mmol, 84% yield) as colorless liquid. Analytical LCMS Condition E: 3.13 min, 333.2 [M−H]⁻.

### Step 2

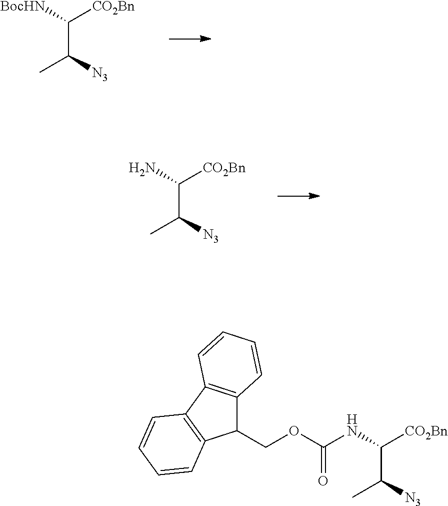 [SMILES: C[C@H](N=[N+]=[N-])[C@H](NC(=O)OC(C)(C)C)C(=O)OCc1ccccc1 ; C[C@H](N=[N+]=[N-])[C@H](N)C(=O)OCc1ccccc1 ; C[C@H](N=[N+]=[N-])[C@H](NC(=O)OCC1c2ccccc2-c2ccccc21)C(=O)OCc1ccccc1]

A solution of benzyl (2S,3S)-3-azido-2-((tert-butoxycarbonyl)amino)butanoate (20 g, 59.8 mmol), dichloromethane (300 mL) and TFA (50 mL, 649 mmol) was stirred for 2 h at 23° C. and then evaporated to dryness to give the corresponding amine. The above amine was redisolved in water (200 mL) and tetrahydrofuran (200 mL). At 0° C., DIPEA (11.49 mL, 65.8 mmol) was added followed by Fmoc chloride (17.02 g, 65.8 mmol). The mixture was warmed up to RT and stirred for 3 h. It was extracted with EtOAc and washed with 0.5 M HCl solution and then brine solution. It was concentrated to get crude liquid. The above crude was purifirf by silica gel column chromatography. The product was eluted at 20% EtOAc in petroleum ether. The fractions were concentrated to get benzyl (2S,3S)-2-((((9H-fluoren-9-yl)methoxy)carbonyl)amino)-3-azidobutanoate (23 g, 50.4 mmol, 84% yield) as a colorless liquid. Analytical LCMS Condition E: 3.70 min, 479.3 [M+Na]⁺.

### Step 3

To a multi-neck round-bottled flask was charged benzyl (2S,3S)-2-((((9H-fluoren-9-yl)methoxy)carbonyl)amino)-3-azidobutanoate (40 g, 88 mmol) in tetrahydrofuran (1200 mL). Pd/C (9.32 g, 8.76 mmol) was added under nitrogen and the reaction was stirred under hydrogen for 12 h. Sodium bicarbonate (11.04 g, 131 mmol) in water 6 (mL) was added followed by Boc-anhydride (30.5 mL, 131 mmol). The mixture was stirring under nitrogen for 12 h. The reaction mass was filtered through celite bed, and washed the bed with THF/Water mixture. The mother liquid was concentrated and washed with EtOAc. Then pH of water layer was adjusted to 7-6 using 1.5 N HCl solution. The resulting white solid was extracted with ethyl acetate. The above reaction was repeated three more times. The combined organics were washed with water and brine solution, dried over sodium sulphate, and concentrated to afford (2S,3S)-2-((((9H-fluoren-9-yl)methoxy)carbonyl)amino)-3-((tert-butoxycarbonyl)amino)butanoic acid as a white solid (28 g). This was mixed with a previously obtained batch (8 g) in DCM (200 mL). n-Hexane (1 L) was added to the above solution and sonicated for 2 min. The solids were filtered, rinsed with hexanes and dried overnight to give (2S,3S)-2-((((9H-fluoren-9-yl)methoxy)carbonyl)amino)-3-((tert-butoxycarbonyl)amino)butanoic acid (36 g, 81 mmol, 92% yield) as a white powder. Analytical LC/MS Condition E. 1.90 min, 439.2 [M−H]⁻. ¹H NMR (400 MHz, DMSO-d₆) δ 7.90 (d, J=7.6 Hz, 2H), 7.75 (d, J=7.2 Hz, 2H), 7.43 (t, J=7.2 Hz, 2H), 7.34 (t, J=Hz, 6.71 (br. d. J=7.6 Hz, 1H), 4.29-4.26 (m, 2H), 4.25-4.21 (m, 1H), 3.94-3.90 (m, 1H), 1.37 (s, 9H), 1.02 (d, J=6.8 Hz, 3H). 13C NMR (101 Hz, DMSO-d₆) δ 171.9, 156.3, 154.8, 143.7, 140.6, 127.6, 127.0, 125.3, 120.0, 77.7, 65.8, 57.8, 47.0, 46.6, 28.2, 16.2.

### Preparation of(S)-2-((((9H-fluoren-9-yl)methoxy)carbonyl)amino)-2-(1-(((tert-butoxycarbonyl)amino)methyl)cyclopropyl)acetic acid

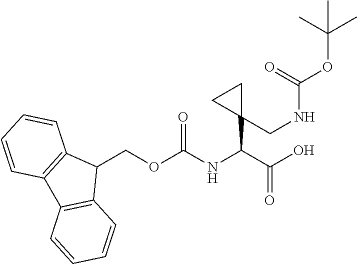 [SMILES: CC(C)(C)OC(=O)NCC1([C@H](NC(=O)OCC2c3ccccc3-c3ccccc32)C(=O)O)CC1]

Final product was obtained following similar procedures of ethyl (S)—S-((tert-butoxycarbonyl)amino)-2-(((S)-mesitylsulfnyl)amino)-3,3-dimethylpentanoate. The synthesis afforded the desired product (0.65 g, 22% yield) as a white solid after purification by flash column chromatography (Red Sep, 40 g, SiO₂, 35 to 40% EtOAc:hexanes (compound ELSD active)). Analytical LC/MS Condition E: 2.04 min, 465.2 [M−H]⁻. ¹H NMR (300 MHz, DMSO-d₆) δ 7.90 (d, J=7.6 Hz, 2H), 7.71 (m, 3H), 7.47-7.27 (m, 2H), 6.98-6.71 (m, 2H), 4.30-4.17 (m, 3H), 3.94-3.82 (m, 1H), 3.20-2.90 (m, 2H), 1.44-1.30 (m, 9H), 0.48 (br s, 4H).

### Preparation of(S)-2-((((9H-fluoren-9-yl)methoxy)carbonyl)amino)-2-(1-(tert-butoxycarbonyl)azetidin-3-yl)acetic acid

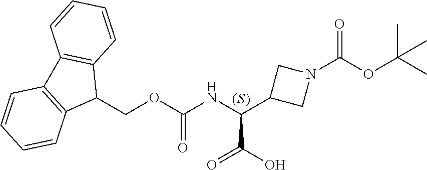

Final product was obtained following similar procedures of ethyl (S)-5-((tert-butoxycarbonyl)amino)-2-(((S)-mesitylsulfinyl)amino)-3,3-dimethylpentanoate. The synthesis afforded the desired product (2.66 g, 20% yield) as a slightly tan solid after purification by reverse-phase HPLC. Analytical LC/MS Condition E. 1.87 min, 467.2 [M−H]⁻. ¹H NMR (400 MHz, DMSO-d₆) δ 7.89 (d, J=7.6 Hz, 2H), 7.69 (m, 2H), 7.41 (t, J=7.2 Hz, 2H), 7.34-7.31 (m, 2H), 6.71 (br. d. J=7.6 Hz, 1H), 4.29-4.23 (m, 3H), 3.77-3.70 (m, 5H), 2.80 (m, 1H), 1.36 (s, 9H).

### Preparation of Example 1000

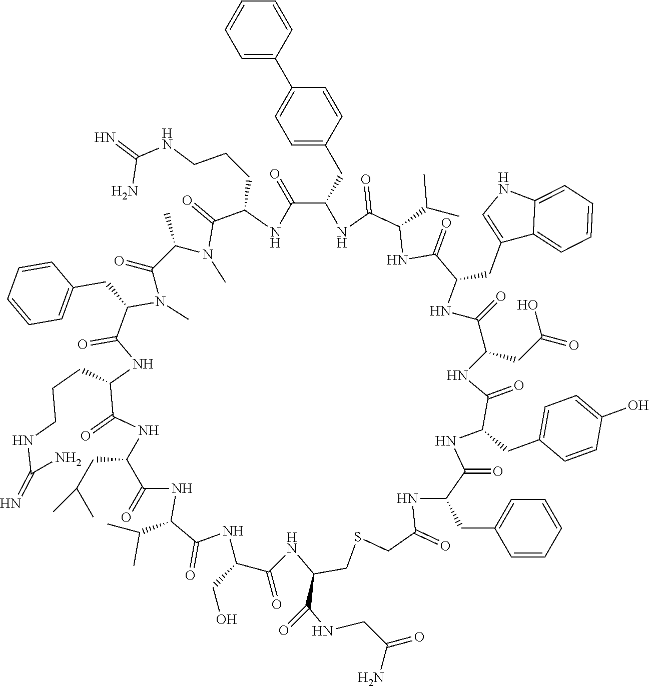 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC1=O]

To a 45-mL polypropylene solid-phase reaction vessel was added using Sieber or Rink resin on a 100 μmol scale, and the reaction vessel was placed on the Symphony peptide synthesizer. The following procedures were then performed sequentially:

“Symphony Resin-swelling procedure” was followed;
“Symphony Single-coupling procedure” was followed with Fmoc-Gly-OH; “Symphony Single-coupling procedure” was followed with Fmoc-Cys(Trt)-OH;
“Symphony Single-coupling procedure” was followed with Fmoc-Ser(tBu)-OH; “Symphony Single-coupling procedure” was followed with Fmoc-Val-OH;
“Symphony Single-coupling procedure” was followed with Fmoc-Leu-OH;
“Symphony Single-coupling procedure” was followed with Fmoc-Arg(Pbf)-OH;
“Symphony Single-coupling procedure” or “Symphony double-coupling procedure” was followed with Fmoc-N-Me-Phe-OH; “Symphony Single-coupling procedure” or “Symphony double-coupling procedure” was followed with Fmoc-N-Me-Gly-OH; “Symphony Single-coupling procedure” was followed with Fmoc-Arg(Pbf)-OH; “Symphony double-coupling procedure” was followed with Fmoc-Bip-OH; “Symphony single-coupling procedure” was followed with Fmoc-Val-OH; “Symphony single-coupling procedure” was followed with Fmoc-Trp(Boc)-OH; “Symphony single-coupling procedure” was followed with Fmoc-Asp(tBu)-OH;
“Symphony Single-coupling procedure” was followed with Fmoc-Tyr(tBu)-OH; “Symphony Single-coupling procedure” was followed with Fmoc-Phe-OH; “Symphony Chloroacetic Anhydride coupling procedure”; “Global Deprotection Method A” was followed; “Cyclization Method” was followed.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 30-70% B over 20 minutes, then a 5-minute hold at 100% B; Flow: 20 mL/min. Fractions containing the desired product were combined and dried via centrifugal evaporation. The material was further purified via preparative LC/MS with the following conditions: Column: XBridge C18, 30×200 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 20-70% B over 20 minutes, then a 2-minute hold at 100% B; Flow: 45 mL/min. Fractions containing the desired product were combined and dried via centrifugal evaporation. The material was further purified via preparative LC/MS with the following conditions: Column: XBridge C18, 30×200 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 20-70% B over 20 minutes, then a 5-minute hold at 100% B; Flow: 45 mL/min. Fractions containing the desired product were combined and dried via centrifugal evaporation. The yield of the product was 1.3 mg, and its estimated purity by LCMS analysis was 100%. Analysis condition A: Retention time=1.74 min; ESI-MS(+) m/z [M+2H]²⁺: 1005.1.

### Preparation of Example 1001

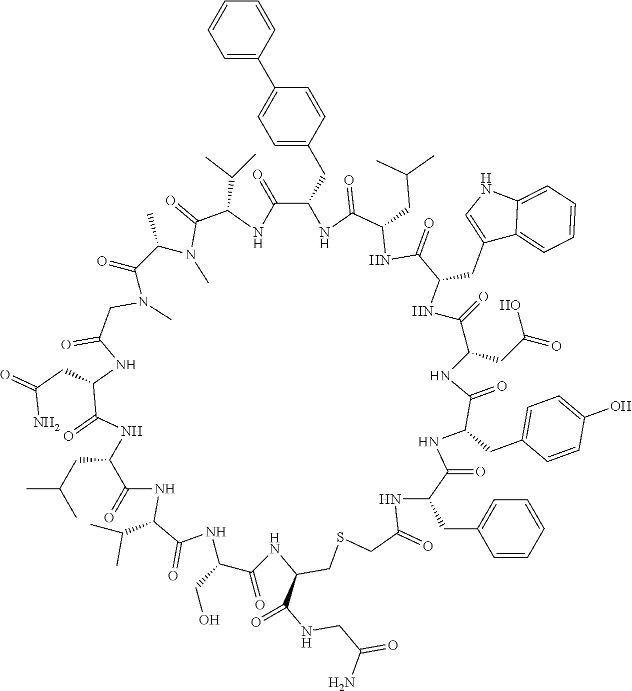 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1001 was prepared, using Sieber or Rink resin on a 100 μmol scale, following the general synthetic sequence described for the preparation of Example 1000 composed of the following general procedures: “Symphony Resin-swelling procedure”, “Symphony Single-coupling procedure”, “Symphony double-coupling procedure”, “Symphony Chloroacetic Anhydride coupling procedure”, “Global Deprotection Method A” and “Cyclization Method”. The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Temperature: 70° C.; Gradient: 20-60% B over 20 minutes, then a 5-minute hold at 100% B; Flow: 20 mL/min. Fractions containing the desired product were combined and dried via centrifugal evaporation. The yield of the product was 11 mg, and its estimated purity by LCMS analysis was 90%. Analysis condition 1: Retention time=1.73 min; ESI-MS(+) m/z [M+2H]²⁺: 918.1.

### Preparation of Example 1002

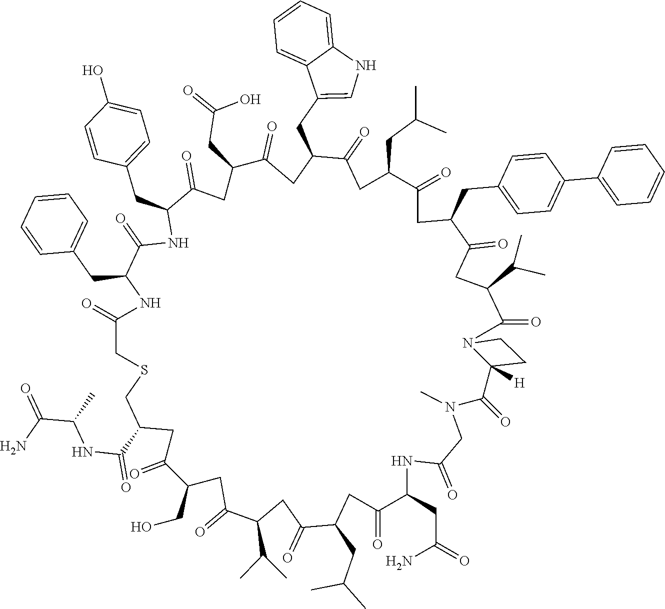 [SMILES: CC(C)C[C@@H]1CC(=O)[C@H](Cc2c[nH]c3ccccc23)CC(=O)[C@H](CC(=O)O)CC(=O)[C@H](Cc2ccc(O)cc2)NC(=O)[C@H](Cc2ccccc2)NC(=O)CSC[C@@H](C(=O)N[C@@H](C)C(N)=O)CC(=O)[C@H](CO)CC(=O)[C@H](C(C)C)CC(=O)[C@H](CC(C)C)CC(=O)[C@H](CC(N)=O)NC(=O)CN(C)C(=O)[C@H]2CCN2C(=O)[C@H](C(C)C)CC(=O)[C@H](Cc2ccc(-c3ccccc3)cc2)CC1=O]

To a 45-mL polypropylene solid-phase reaction vessel was added Sieber resin or Rink resin (70 mg for Sieber or 100 mg for Rink, 0.050 mmol), and the reaction vessel was placed on the Symphony X peptide synthesizer. The following procedures were then performed sequentially:

“Symphony X Resin-swelling procedure” was followed; “Symphony X Single-coupling procedure” was followed with Fmoc-Ala-OH; “Symphony X Single-coupling procedure” was followed with Fmoc-Cys(Trt)-OH; “Symphony X Single-coupling procedure” was followed with Fmoc-Ser(tBu)-OH; “Symphony X Single-coupling procedure” was followed with Fmoc-Val-OH; “Symphony X Single-coupling procedure” was followed with Fmoc-Leu-OH; “Symphony X Single-coupling procedure” was followed with Fmoc-Asn(Trt)-OH; “Symphony X Single-coupling procedure” was followed with Fmoc-N-Me-Gly(or Sar)-OH; “Symphony X Single-Coupling Manual Addition Procedure B” was followed with Fmoc-D-Azt-OH; “Symphony X Single-coupling procedure” was followed with Fmoc-Val-OH; “Symphony X Single-coupling procedure” was followed with Fmoc-Bip-OH; “Symphony X Single-coupling procedure” was followed with Fmoc-Leu-OH; “Symphony X Single-coupling procedure” was followed with Fmoc-Trp(Boc)-OH;
“Symphony X Single-coupling procedure” was followed with Fmoc-Asp(tBu)-OH; “Symphony X Single-coupling procedure” was followed with Tyr(tBu)-OH; “Symphony X Single-coupling procedure” was followed with Fmoc-Phe-OH; “Symphony X Chloroacetic Anhydride coupling procedure” was followed; “Symphony X Final rinse and dry procedure” was followed; “Global Deprotection Method A” was followed; “Cyclization Method A” was followed.

The crude material was purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-65% B over 20 minutes, then a 2-minute hold at 100% B; Flow: 45 mL/min. Fractions containing the desired product were combined and dried via centrifugal evaporation. The yield of the product was 44.5 mg, and its estimated purity by LCMS analysis was 100%.
Analysis condition A: Retention time=1.61 min; ESI-MS(+) m/z [M+H]⁺: 1846.0
Analysis condition B: Retention time=1.77 min; ESI-MS(+) m/z [M+H]⁺: 1846.1.

### Preparation of Example 1003

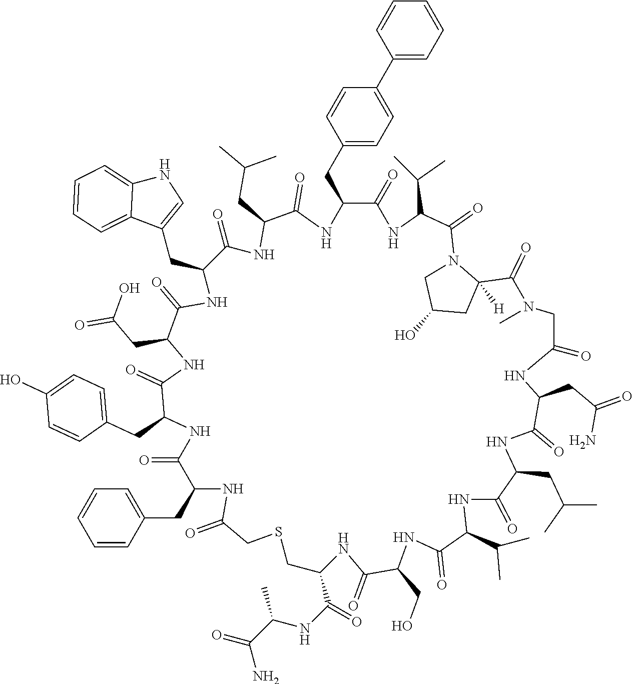

Example 1003 was prepared, using Sieber or Rink resin on a 50 μmol scale, following the general synthetic sequence described for the preparation of Example 1002, composed of the following general procedures: “Symphony X Resin-swelling procedure”, “Symphony X Single-coupling procedure”; “Symphony X Single-Coupling Manual Addition Procedure B” was followed with Fmoc-D-Hyp-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×150 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 15-65% B over 20 minutes, then a 2-minute hold at 100% B; Flow: 40 mL/min. Fractions containing the desired product were combined and dried via centrifugal evaporation. The yield of the product was 3.2 mg, and its estimated purity by LCMS analysis was 82.3%.
Analysis condition A: Retention time=1.49 min; ESI-MS(+) m/z [M+2H]²⁺: 939.1.

### Preparation of Example 1004

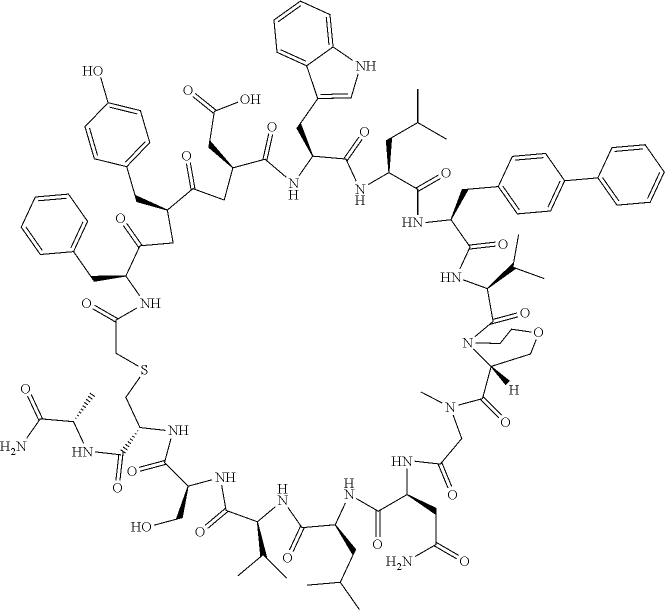

Example 1004 was prepared, using Sieber or Rink on a 50 μmol scale, following the general synthetic sequence described for the preparation of Example 1002 composed of the following general procedures: composed of the following general procedures: “Symphony X Resin-swelling procedure”. “Symphony X Single-coupling procedure”, “Symphony X Single-Coupling Manual Addition Procedure B” was followed with Fmoc-D-Mor-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×150 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 15-65% B over 20 minutes, then a 2-minute hold at 100% B; Flow: 40 mL/min. Fractions containing the desired product were combined and dried via centrifugal evaporation. The yield of the product was 34 mg, and its estimated purity by LCMS analysis was 100%.
Analysis condition A: Retention time=1.55 min; ESI-MS(+) m/z [M+H]⁺: 1877.0.
Analysis condition B: Retention time=1.71 min; ESI-MS(+) m/z [M+2H]²⁺: 939.0.

### Preparation of Example 1005

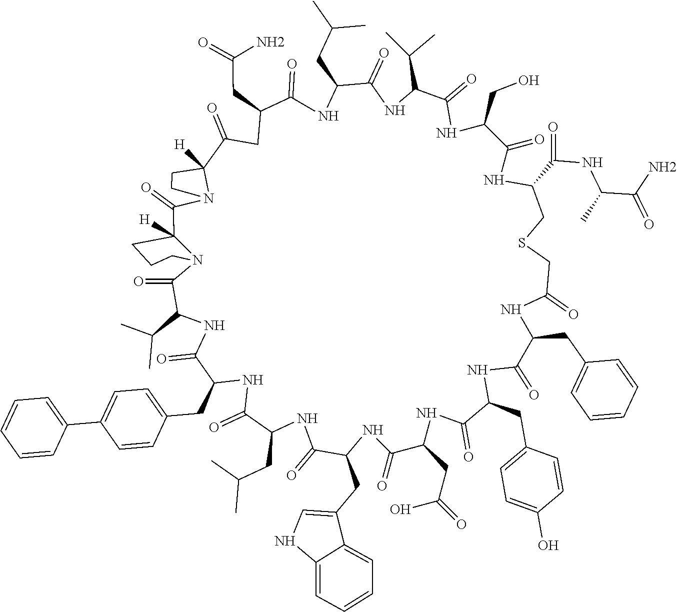

Example 1005 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”,

“Symphony X Single-Coupling Manual Addition Procedure B” was followed with Fmoc-D-Azt-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-65% B over 20 minutes, then a 2-minute hold at 100% B; Flow: 45 mL/min. Fractions containing the desired product were combined and dried via centrifugal evaporation. The yield of the product was 34.9 mg, and its estimated purity by LCMS analysis was 100%.
Analysis condition A: Retention time=1.51 min; ESI-MS(+) m/z [M+2H]²⁺: 936.9.
Analysis condition B: Retention time=1.69 min; ESI-MS(+) m/z [M+2H]²⁺: 937.1.

### Preparation of Example 1006

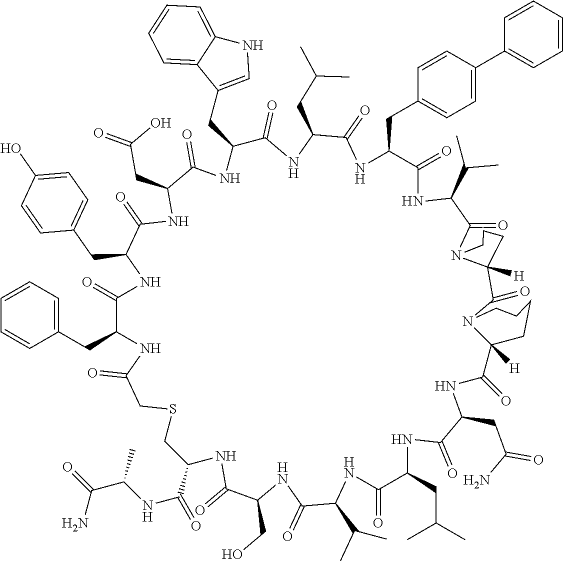

Example 1006 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”,

“Symphony X Single-Coupling Manual Addition Procedure B” was followed with Fmoc-D-Mor-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-65% B over 20 minutes, then a 2-minute hold at 100% B; Flow: 45 mL/min. Fractions containing the desired product were combined and dried via centrifugal evaporation. The yield of the product was 27.5 mg, and its estimated purity by LCMS analysis was 100%.
Analysis condition A: Retention time=1.73 min; ESI-MS(+) m/z [M+H]⁺: 1900.1.
Analysis condition B: Retention time=1.92 min; ESI-MS(+) m/z [M+H]⁺: 1900.1.

### Preparation of Example 1007

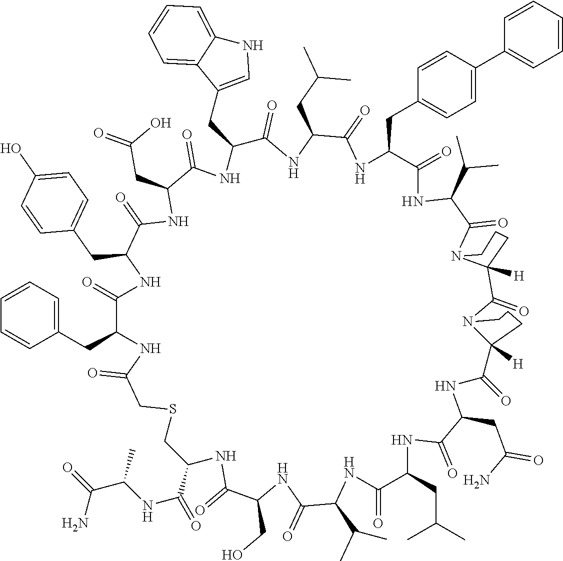

Example 1007 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, “Symphony double-coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 18-58% B over 20 minutes, then a 3-minute hold at 100% B; Flow: 20 mL/min. Fractions containing the desired product were combined and dried via centrifugal evaporation. The yield of the product was 37.2 mg, and its estimated purity by LCMS analysis was 98.2%.
Analysis condition A: Retention time=1.67 min; ESI-MS(+) m/z [M−H]⁻: 1885.9.
Analysis condition B: Retention time=1.86 min; ESI-MS(+) m/z [M+H]⁺: 1887.1.

### Preparation of Example 1008

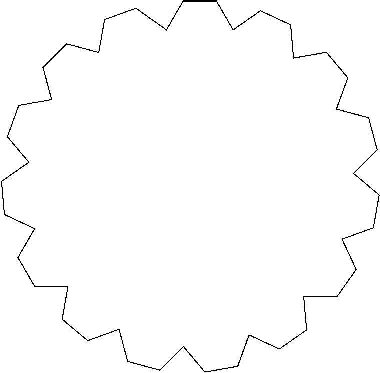 [SMILES: C1CCCCCCCCCCCCCCCCCCCCCCCCCCCCCCCCCCCCCCCCCCCC1]

Example 1008 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Single-Coupling Manual Addition Procedure B” was followed with Fmoc-D-Mor-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-65% B over 20 minutes, then a 2-minute hold at 100% B; Flow: 45 mL/min. Fractions containing the desired product were combined and dried via centrifugal evaporation. The yield of the product was 36.3 mg, and its estimated purity by LCMS analysis was 100%.
Analysis condition A: Retention time=1.66 min; ESI-MS(+) m/z [M+H]⁺: 1901.9.
Analysis condition B: Retention time=1.84 min; ESI-MS(+) m/z [M+H]⁺: 1902.1.

### Preparation of Example 1009

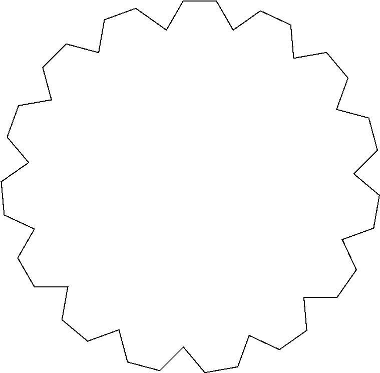 [SMILES: C1CCCCCCCCCCCCCCCCCCCCCCCCCCCCCCCCCCCCCCCCCCCC1]

Example 1009 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Single-Coupling Manual Addition Procedure B” was followed with Fmoc-D-Hyp-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×150 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 15-65% B over 20 minutes, then a 2-minute hold at 100% B; Flow: 40 mL/min. Fractions containing the desired product were combined and dried via centrifugal evaporation. The yield of the product was 15.9 mg, and its estimated purity by LCMS analysis was 92.2%.
Analysis condition A: Retention time=1.54 min; ESI-MS(+) m/z [M+2H]²⁺: 951.1.
Analysis condition B: Retention time=1.72 min; ESI-MS(+) m/z [M+2H]²⁺: 951.7.

### Preparation of Example 1010

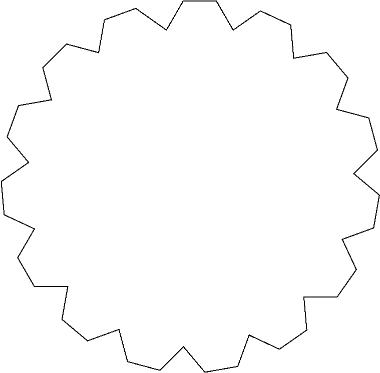 [SMILES: C1CCCCCCCCCCCCCCCCCCCCCCCCCCCCCCCCCCCCCCCCCCCC1]

Example 1010 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, “Symphony double-coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 20 minutes, then a 5-minute hold at 100% B; Flow: 20 mL/min. Fractions containing the desired product were combined and dried via centrifugal evaporation. The yield of the product was 5.5 mg, and its estimated purity by LCMS analysis was 100%.
Analysis condition A: Retention time=1.55 min; ESI-MS(+) m/z [M+H]⁺: 1846.1.
Analysis condition B: Retention time=1.71 min: ESI-MS(+) m/z [M+H]⁺: 1846.1.

### Preparation of Example 1011

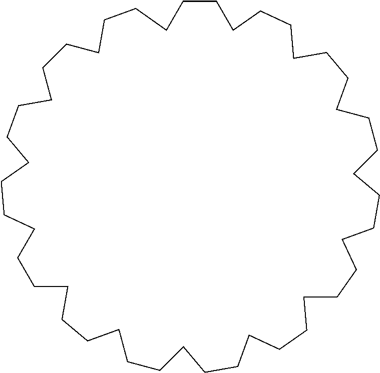 [SMILES: C1CCCCCCCCCCCCCCCCCCCCCCCCCCCCCCCCCCCCCCCCCCCC1]

Example 1011 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, Single-Coupling Pre-Activation Procedure “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 19 minutes, then a 5-minute hold at 100% B; Flow: 20 mL/min. Fractions containing the desired product were combined and dried via centrifugal evaporation. The yield of the product was 37.7 mg, and its estimated purity by LCMS analysis was 100%.
Analysis condition A: Retention time=1.52 min; ESI-MS(+) m/z [M+2H]²⁺: 937.1.
Analysis condition B: Retention time=1.67 min; ESI-MS(+) m/z [M+2H]²⁺: 937.2.

### Preparation of Example 1012

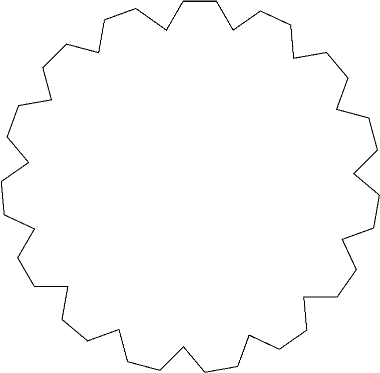 [SMILES: C1CCCCCCCCCCCCCCCCCCCCCCCCCCCCCCCCCCCCCCCCCCCC1]

Example 1012 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, “Symphony double-coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-55% B over 25 minutes, then a 5-minute hold at 100% B; Flow: 20 mL/min. Fractions containing the desired product were combined and dried via centrifugal evaporation. The yield of the product was 9.4 mg, and its estimated purity by LCMS analysis was 98.6%.
Analysis condition A: Retention time=1.37 min; ESI-MS(+) m/z [M+2H]²⁺: 933.3.
Analysis condition B: Retention time=1.48 min; ESI-MS(+) m/z [M+2H]²⁺: 933.2.

### Preparation of Example 1013

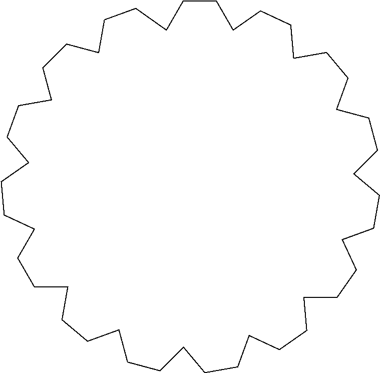 [SMILES: C1CCCCCCCCCCCCCCCCCCCCCCCCCCCCCCCCCCCCCCCCCCCC1]

Example 1013 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×150 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 20-70% B over 20 minutes, then a 2-minute hold at 100% B; Flow: 40 mL/min. Fractions containing the desired product were combined and dried via centrifugal evaporation. The yield of the product was 18.7 mg, and its estimated purity by LCMS analysis was 99.2%.
Analysis condition A: Retention time=1.59 min; ESI-MS(+) m/z [M+H]⁺: 1831.1.
Analysis condition B: Retention time=1.78 min; ESI-MS(+) m/z [M+H]⁺: 1831.2.

### Preparation of Example 1014

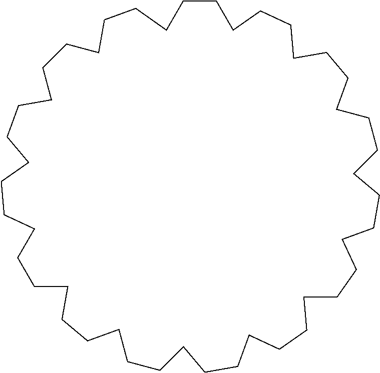 [SMILES: C1CCCCCCCCCCCCCCCCCCCCCCCCCCCCCCCCCCCCCCCCCCCC1]

Example 1014 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, Single-Coupling Pre-Activation Procedure” for Fmoc-D-Pro(4-NHBoc)-OH, “Symphony Chloroacetic Anhydride coupling procedure”, “Global Deprotection Method A” and “Cyclization Method”.

The crude material was purified via preparative LC/MS with the following conditions: Column: XBridge C18, 30×200 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 5-55% B over 20 minutes, then a 2-minute hold at 100% B; Flow: 40 mL/min. Fractions containing the desired product were combined and dried via centrifugal evaporation. The yield of the product was 5.2 mg, and its estimated purity by LCMS analysis was 97.5%.
Analysis condition A: Retention time=1.52 min; ESI-MS(+) m/z [M+2H]²⁺: 938.2.
Analysis condition B: Retention time=1.49 min; ESI-MS(+) m/z [M+2H]²⁺: 938.1.

### Preparation of Example 1015

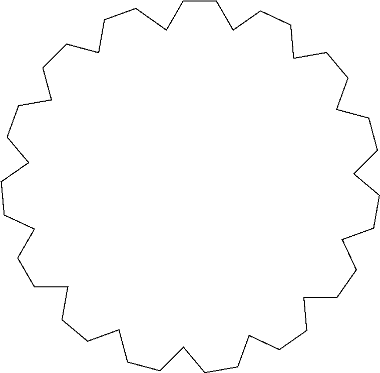 [SMILES: C1CCCCCCCCCCCCCCCCCCCCCCCCCCCCCCCCCCCCCCCCCCCC1]

Example 1015 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, Single-Coupling Pre-Activation Procedure” for Fmoc-Tyr(3-NO2)-OH, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 2-60% B over 20 minutes, then a 5-minute hold at 100% B; Flow: 20 mL/min. Fractions containing the desired product were combined and dried via centrifugal evaporation. The yield of the product was 5.8 mg, and its estimated purity by LCMS analysis was 95%.
Analysis condition A: Retention time=1.70 min; ESI-MS(+) m/z [M+H]⁺: 1920.1.
Analysis condition B: Retention time=1.89 min; ESI-MS(+) m/z [M+Na]⁺: 1941.1.

### Preparation of Example 1016

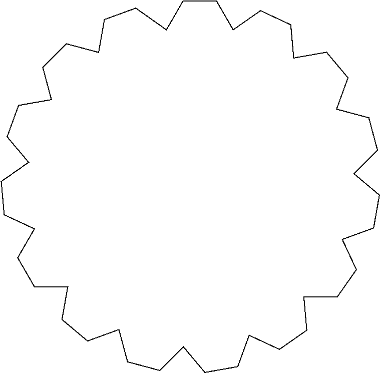 [SMILES: C1CCCCCCCCCCCCCCCCCCCCCCCCCCCCCCCCCCCCCCCCCCCC1]

Example 1016 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×150 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 15-65% B over 20 minutes, then a 2-minute hold at 100% B; Flow: 40 mL/min. Fractions containing the desired product were combined and dried via centrifugal evaporation. The yield of the product was 16.9 mg, and its estimated purity by LCMS analysis was 98.8%.
Analysis condition A: Retention time=1.61 min; ESI-MS(+) m/z [M+H]⁺: 1888.0.
Analysis condition B: Retention time=1.67 min; ESI-MS(+) m/z [M+H]⁺: 1887.8.

### Preparation of Example 1017

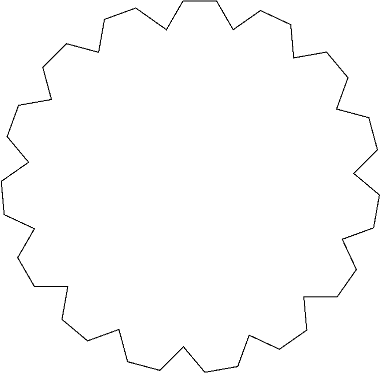 [SMILES: C1CCCCCCCCCCCCCCCCCCCCCCCCCCCCCCCCCCCCCCCCCCCC1]

Example 1017 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, Single-Coupling Pre-Activation Procedure” for Fmoc-Phe (4-CONH₂)—OH, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-60% B over 20 minutes, then a 5-minute hold at 100% B; Flow: 20 mL/min. Fractions containing the desired product were combined and dried via centrifugal evaporation. The yield of the product was 28 mg, and its estimated purity by LCMS analysis was 100%.
Analysis condition A: Retention time=1.53 min; ESI-MS(+) m/z [M+H]⁺: 1902.2.
Analysis condition B: Retention time=1.71 min; ESI-MS(+) m/z [M−H]: 1898.5.

### Preparation of Example 1018

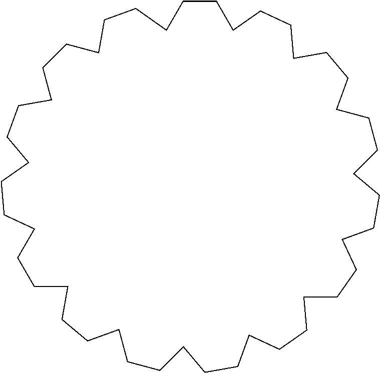 [SMILES: C1CCCCCCCCCCCCCCCCCCCCCCCCCCCCCCCCCCCCCCCCCCCC1]

Example 1018 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, Single-Coupling Pre-Activation Procedure” for Fmoc-Phe(3-Cl)—OH, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 30-70% B over 20 minutes, then a 4-minute hold at 100% B; Flow: 20 mL/min. Fractions containing the desired product were combined and dried via centrifugal evaporation. The yield of the product was 25.2 mg, and its estimated purity by LCMS analysis was 98.7%.
Analysis condition A: Retention time=2.11 min; ESI-MS(+) m/z [M+H]⁺: 1892.1.
Analysis condition B: Retention time=2.0 min; ESI-MS(+) m/z [M+H]⁺: 1892.0.

### Preparation of Example 1019

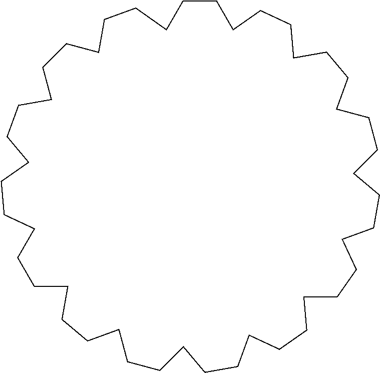 [SMILES: C1CCCCCCCCCCCCCCCCCCCCCCCCCCCCCCCCCCCCCCCCCCCC1]

Example 1019 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, Single-Coupling Pre-Activation Procedure” for Fmoc-3-Pyr-OH, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 19 minutes, then a 5-minute hold at 100% B; Flow: 20 mL/min. Fractions containing the desired product were combined and dried via centrifugal evaporation. The yield of the product was 24.4 mg, and its estimated purity by LCMS analysis was 100%.
Analysis condition A: Retention time=1.14, 1.44 min; ESI-MS(+) m/z [M+2H]²⁺: 938.17, 938.12.
Analysis condition B: Retention time=1.49 min; ESI-MS(+) m/z [M+2H]²⁺: 938.3.

### Preparation of Example 1020

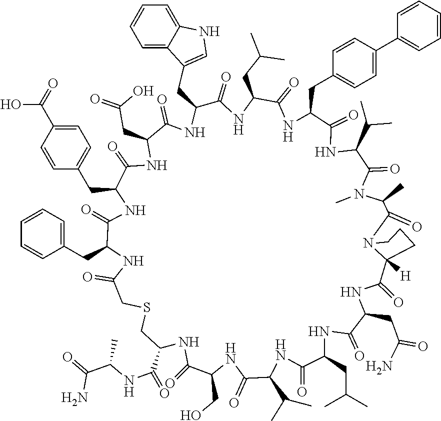

Example 1020 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Single-Coupling Manual Addition Procedure B” was followed with Fmoc-Phe(4-COOtBu)-OH; “Symphony X Chloroacetic Anhydride coupling procedure”, “Symphony X Final rinse and dry procedure”, “Global Deprotection Method A”, “Cyclization Method A”. ridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-55% B over 20 minutes, then a 5-minute hold at 100% B; Flow: 20 mL/min. Fractions containing the desired product were combined and dried via centrifugal evaporation. The yield of the product was 28.1 mg, and its estimated purity by LCMS analysis was 100%.
Analysis condition A: Retention time=1.42 min; ESI-MS(+) m/z [M+H]⁺: 1901.9.
Analysis condition B: Retention time=1.74 min; ESI-MS(+) m/z [M+2H]²⁺: 952.1.

### Preparation of Example 1021

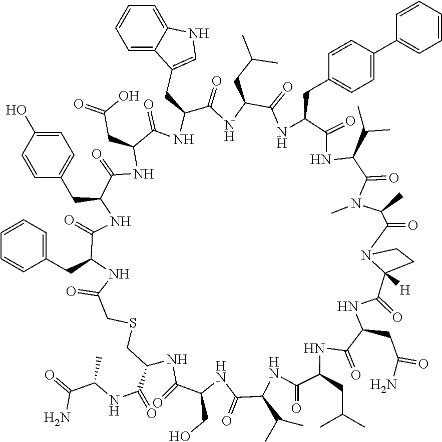 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)[C@H]2CCN2C(=O)[C@H](C)N(C)C(=O)[C@H](C(C)C)NC(=O)[C@H](Cc2ccc(-c3ccccc3)cc2)NC1=O]

Example 1021 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”,

“Symphony X Single-Coupling Manual Addition Procedure B” was followed with Fmoc-D-Azt-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×150 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 15-65% B over 20 minutes, then a 2-minute hold at 100% B; Flow: 40 mL/min. Fractions containing the desired product were combined and dried via centrifugal evaporation. The yield of the product was 27.7 mg, and its estimated purity by LCMS analysis was 97.6%.

Analysis condition 2: Retention time=1.44 min; ESI-MS(+) m/z [M+2H]²⁺: 930.1. Analysis condition 2: Retention time=1.62 min; ESI-MS(+) m/z [M+2H]²⁺: 930.7.

### Preparation of Example 1022

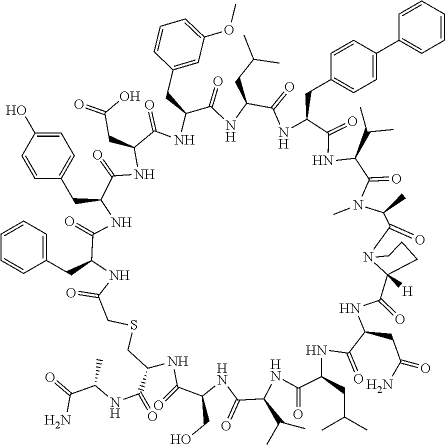

Example 1022 was prepared, using Sieber or Rink on a 50 μmol scale, following the general synthetic sequence described for the preparation of Example 1054, composed of the following general procedures: “Prelude Resin-swelling procedure”, “Prelude Single-coupling procedure”, “Symphony Resin-swelling procedure”, “Symphony Single-coupling procedure”, “Symphony Single-Coupling Pre-activation Procedure” was followed with Fmoc-Phe(3-OMe)-OH; “Symphony Chloroacetic Anhydride coupling procedure”, “Global Deprotection Method A”, “Cyclization Method A”.

The crude material was purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 5-55% B over 20 minutes, then a 2-minute hold at 100% B; Flow: 45 mL/min. Fractions containing the desired product were combined and dried via centrifugal evaporation. The yield of the product was 24.6 mg, and its estimated purity by LCMS analysis was 98.9%.
Analysis condition A: Retention time=1.71, 1.75 min; ESI-MS(+) m/z [M+NH₄]⁺: 1882.5.
Analysis condition B: Retention time=1.85 min; ESI-MS(+) m/z [M+H]⁺: 1865.2.

### Preparation of Example 1023

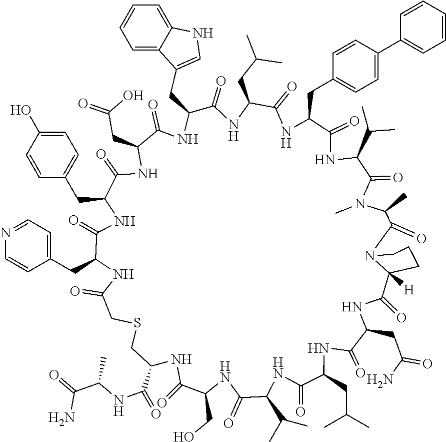

Example 1023 was prepared, using Sieber or Rink on a 50 μmol scale, following the general synthetic sequence described for the preparation of Example 1002 and Example 1000, composed of the following general procedures: “Symphony X Resin-swelling procedure”, “Symphony X Single-coupling procedure”, “Single-Coupling Pre-Activation Procedure” was followed with Fmoc-4-Pyr-OH; “Symphony Chloroacetic Anhydride coupling procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 15-55% B over 25 minutes, then a 5-minute hold at 100% B; Flow: 20 mL/min. Fractions containing the desired product were combined and dried via centrifugal evaporation. The yield of the product was 17.2 mg, and its estimated purity by LCMS analysis was 98.3%.
Analysis condition A: Retention time=1.5 min; ESI-MS(+) m/z [M+H]⁺: 1875.1.
Analysis condition B: Retention time=1.59 min; ESI-MS(+) m/z [M+H]⁺: 1875.0.

### Preparation of Example 1024

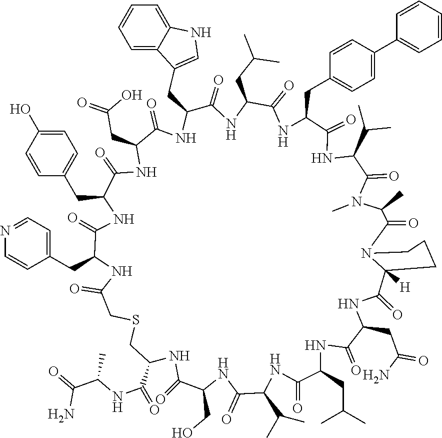

Example 1024 was prepared, using Sieber or Rink on a 50 μmol scale, following the general synthetic sequence described for the preparation of Example 1002 and Example 1000, composed of the following general procedures: “Symphony X Resin-swelling procedure”, “Symphony X Single-coupling procedure”, “Single-Coupling Pre-Activation Procedure” was followed with Fmoc-4-Pyr-OH; “Symphony Chloroacetic Anhydride coupling procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 13-53% B over 20 minutes, then a 5-minute hold at 100% Flow: 20 mL/min. Fractions containing the desired product were combined and dried via centrifugal evaporation. The yield of the product was 27.6 mg, and its estimated purity by LCMS analysis was 100%.
Analysis condition A: Retention time=1.43 min; ESI-MS(+) m/z [M+2H]²⁺: 931.2.
Analysis condition B: Retention time=1.45 min; ESI-MS(+) m/z [M+2H]²⁺: 931.2.

### Preparation of Example 1025

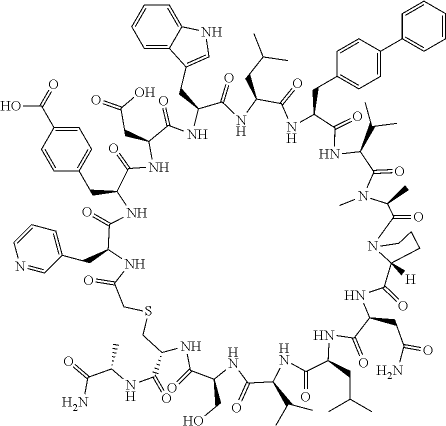

Example 1025 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”,

“Symphony X Single-Coupling Manual Addition Procedure B” was followed with Fmoc-Phe(4-COOtBu)-OH and Fmoc-Ala(3-Pyr)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 7-47% B over 25 minutes, then a 5-minute hold at 100% B; Flow: 20 mL/min. Fractions containing the desired product were combined and dried via centrifugal evaporation. The yield of the product was 33 mg, and its estimated purity by LCMS analysis was 100%.
Analysis condition A: Retention time=1.54 min; ESI-MS(+) m/z [M+H]⁺: 1903.6.
Analysis condition B: Retention time=1.53 min; ESI-MS(+)m z [M+H]⁺: 1903.6.

### Preparation of Example 1026

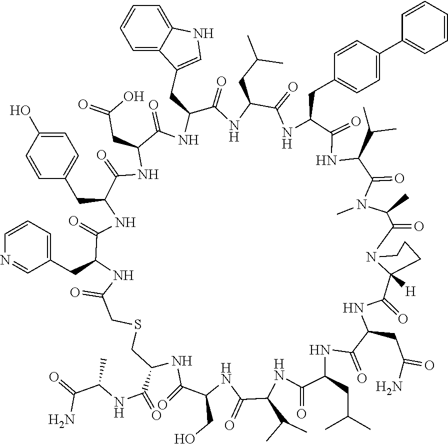

Example 1026 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, Single-Coupling Pre-Activation Procedure” for Fmoc-Ala(3-Pyr)-OH, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 17-57% B over 25 minutes, then a 5-minute hold at 100% B; Flow: 20 mL/min. Fractions containing the desired product were combined and dried via centrifugal evaporation. The yield of the product was 19.8 mg, and its estimated purity by LCMS analysis was 97.8%.
Analysis condition A: Retention time=1.44 min; ESI-MS(+) m/z [M+2H]²⁺: 931.1.
Analysis condition B: Retention time=1.58 min; ESI-MS(+) m/z [M+2H]²⁺: 931.1.

### Preparation of Example 1027

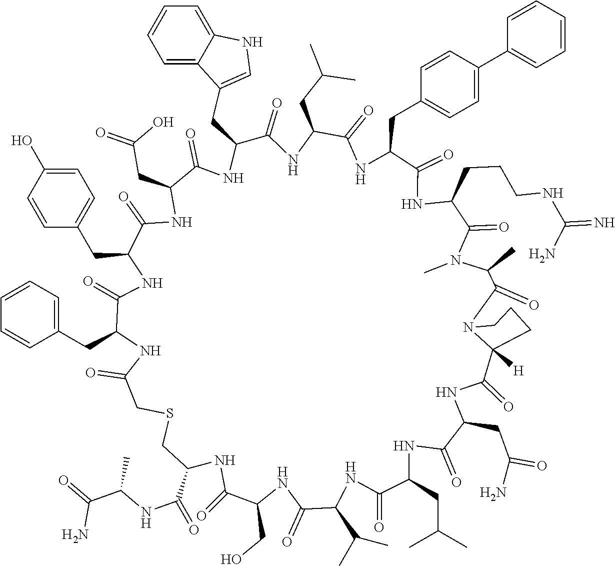

Example 1027 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 20 minutes, then a 3-minute hold at 100% B; Flow: 20 mL/min. Fractions containing the desired product were combined and dried via centrifugal evaporation. The yield of the product was 34 mg, and its estimated purity by LCMS analysis was 94.7%.
Analysis condition A: Retention time=1.75 min; ESI-MS(+) m/z [M+H]⁺: 1931.9.
Analysis condition B: Retention time=1.77 min; ESI-MS(+) m/z [M+H]⁺: 1930.9.

### Preparation of Example 1028

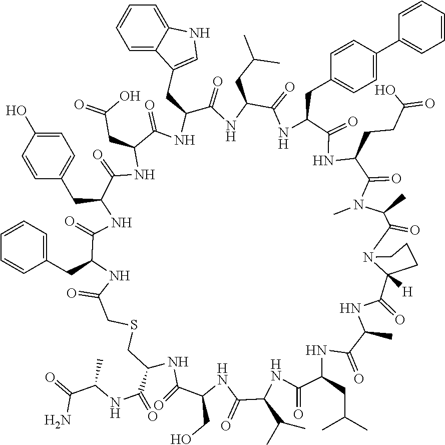

Example 1028 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 10-60% B over 20 minutes, then a 2-minute hold at 100% B; Flow: 45 mL/min. Fractions containing the desired product were combined and dried via centrifugal evaporation. The yield of the product was 27.9 mg, and its estimated purity by LCMS analysis was 100%.
Analysis condition A: Retention time=1.48, 1.51 min; ESI-MS(+) m/z [M+H]⁺: 1861.1, 1861.1.
Analysis condition B: Retention time=1.78 min; ESI-MS(+) m/z [M+H]⁺: 1861.1.

### Preparation of Example 1029

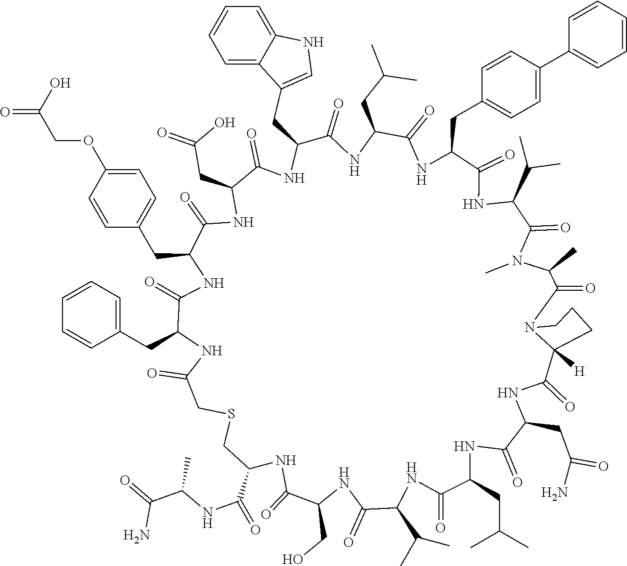

Example 1029 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, Single-Coupling Pre-Activation Procedure” for Fmoc-Tyr(CH₂COOtBu)-OH, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-55% B over 20 minutes, then a 5-minute hold at 100% Flow: 20 mL/min. Fractions containing the desired product were combined and dried via centrifugal evaporation. The yield of the product was 15.3 mg, and its estimated purity by LCMS analysis was 96.7%.
Analysis condition A: Retention time=1.33 min; ESI-MS(+) m/z [M+2H]²⁺: 967.0.
Analysis condition B: Retention time=1.68 min; ESI-MS(+) m/z [M+2H]²⁺: 967.1.

### Preparation of Example 1030

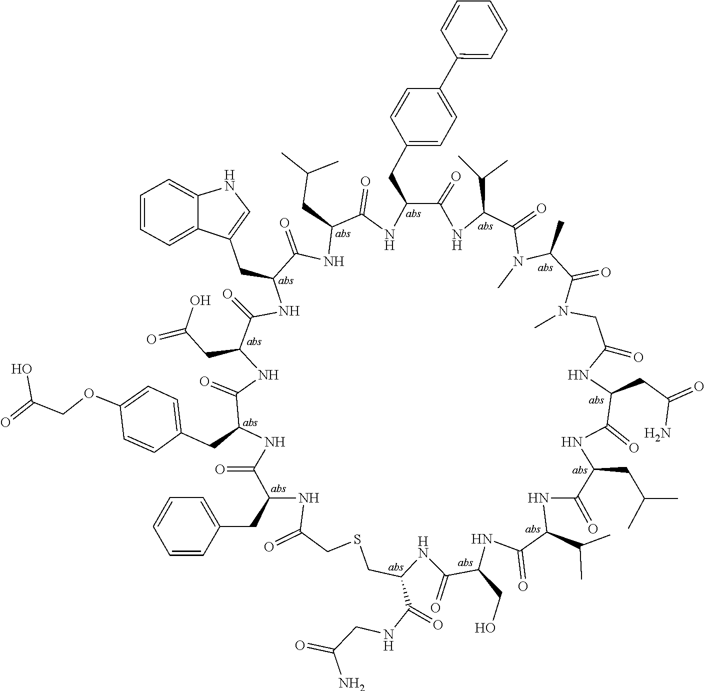 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1030 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×150 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 15-65% B over 20 minutes, then a 2-minute hold at 100% B; Flow: 40 mL/min. Fractions containing the desired product were combined and dried via centrifugal evaporation. The yield of the product was 40.6 mg, and its estimated purity by LCMS analysis was 94.1%.
Analysis condition A: Retention time=1.41 min; ESI-MS(+) m/z [M+H]⁺: 1892.2.
Analysis condition B: Retention time=1.75 min; ESI-MS(+) m/z [M+2H]²⁺: 947.1.

### Preparation of Example 1031

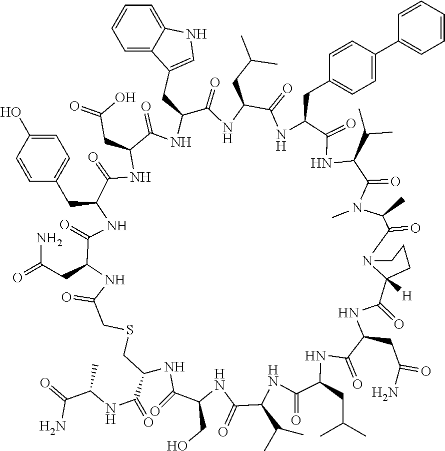

Example 1031 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, “Symphony double-coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-55% B over 25 minutes, then a 5-minute hold at 100% B; Flow: 20 mL/min. Fractions containing the desired product were combined and dried via centrifugal evaporation. The yield of the product was 29.3 mg, and its estimated purity by LCMS analysis was 95.4%.
Analysis condition A: Retention time=1.42 min; ESI-MS(+) m/z [M+2H]²⁺: 921.1.
Analysis condition B: Retention time=1.5 min; ESI-MS(+) m/z [M+2H]²⁺: 921.3.

### Preparation of Example 1032

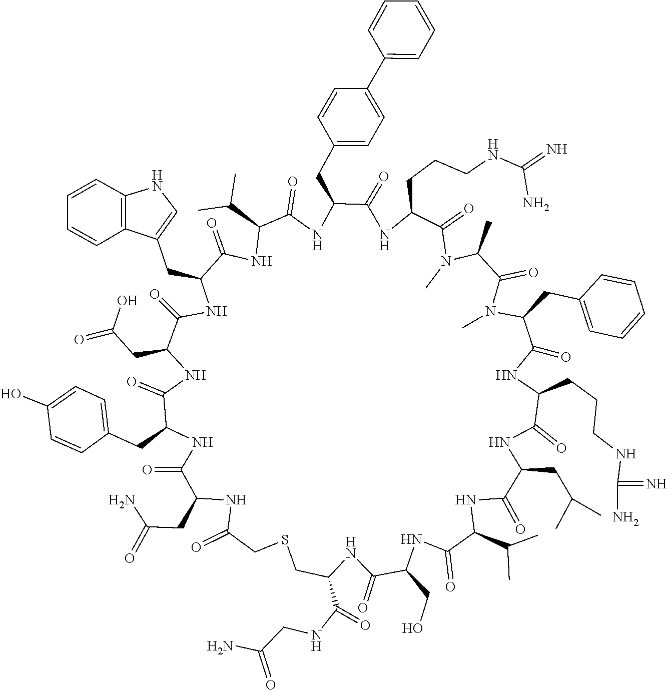 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)NCC(N)=O)NC(=O)[C@H](CO)NC(=O)[C@H](C(C)C)NC1=O]

Example 1032 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-65% B over 20 minutes, then a 2-minute hold at 100% B; Flow: 45 mL/min. Fractions containing the desired product were combined and dried via centrifugal evaporation. The yield of the product was 24.9 mg, and its estimated purity by LCMS analysis was 98.6%.
Analysis condition A: Retention time=1.68 min; ESI-MS(+) m/z [M+2H]²⁺: 989.0.
Analysis condition B: Retention time=1.67 min; ESI-MS(+) m/z [M+2H]²⁺: 989.0.

### Preparation of Example 1033

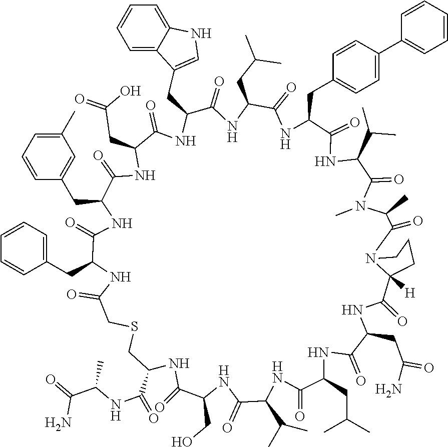

Example 1033 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, Single-Coupling Pre-Activation Procedure” for Fmoc-Phe(3-Me)-OH, “Symphony Chloroacetic Anhydride coupling procedure”, “Global Deprotection Method A” and “Cyclization Method”.

The crude material was purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-70% B over 20 minutes, then a 2-minute hold at 100% B; Flow: 45 mL/min. Fractions containing the desired product were combined and dried via centrifugal evaporation. The yield of the product was 33.9 mg, and its estimated purity by LCMS analysis was 98.4%.
Analysis condition A: Retention time=1.8 min; ESI-MS(+) m/z [M+H]⁺: 1873.2.
Analysis condition B: Retention time=1.98 min; ESI-MS(+) m/z [M+H]⁺: 1872.3.

### Preparation of Example 1034

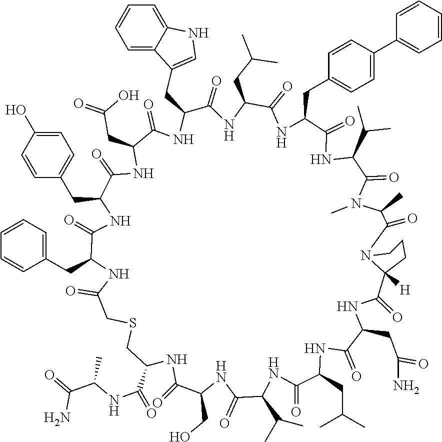

Example 1034 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×200 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 20-70% B over 20 minutes, then a 2-minute hold at 100% B; Flow: 40 mL/min. Fractions containing the desired product were combined and dried via centrifugal evaporation. The yield of the product was 27.7 mg, and its estimated purity by LCMS analysis was 95.2%.
Analysis condition A: Retention time=1.66 min; ESI-MS(+) m/z [M+H]⁺: 1873.7.
Analysis condition B: Retention time=1.82 min; ESI-MS(+) m/z [M+H]⁺: 1873.7.

### Preparation of Example 1035

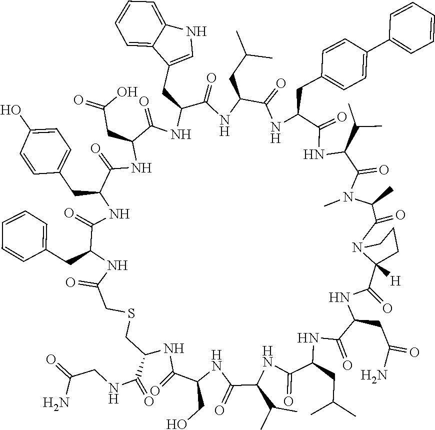

Example 1035 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, “Symphony double-coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 20 minutes, then a 5-minute hold at 100% B; Flow: 20 mL/min. Fractions containing the desired product were combined and dried via centrifugal evaporation. The yield of the product was 36.7 mg, and its estimated purity by LCMS analysis was 100%.
Analysis condition A: Retention time=1.53 min; ESI-MS(+) m/z [M+2H]²⁺: 931.2.
Analysis condition B: Retention time=1.69 min; ESI-MS(+) m/z [M+2H]²⁺: 931.2.

### Preparation of Example 1036

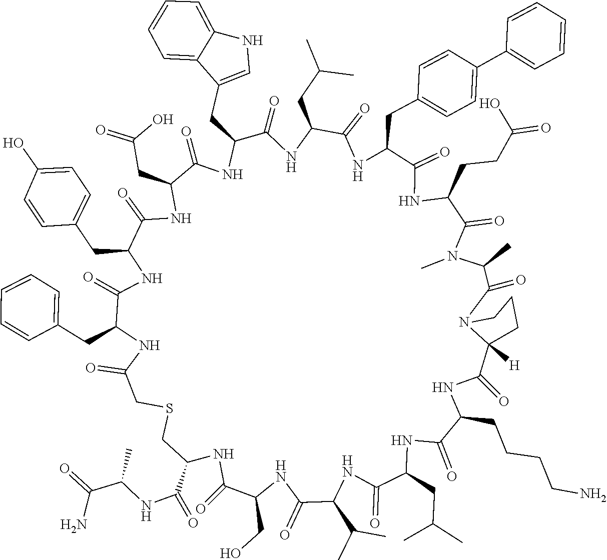

Example 1036 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-55% B over 20 minutes, then a 5-minute hold at 100% B; Flow: 20 mL/min. Fractions containing the desired product were combined and dried via centrifugal evaporation. The yield of the product was 14.9 mg, and its estimated purity by LCMS analysis was 99.3%.
Analysis condition A: Retention time=1.49 min; ESI-MS(+) m/z [M+H]⁺: 1919.2.
Analysis condition B: Retention time=1.64 min; ESI-MS(+) m/z [M+H]⁺: 1919.1.

### Preparation of Example 1037

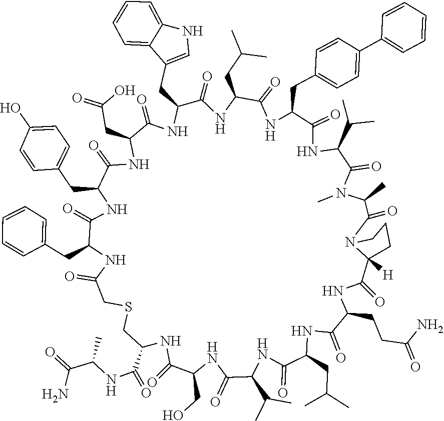

Example 1037 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 10-60% B over 20 minutes, then a 2-minute hold at 100% B; Flow: 45 mL/min. Fractions containing the desired product were combined and dried via centrifugal evaporation. The yield of the product was 15.1 mg, and its estimated purity by LCMS analysis was 96.9%.
Analysis condition A: Retention time=1.38 min; ESI-MS(+) m/z [M+2H]²⁺: 945.1.
Analysis condition B: Retention time=1.69 min; ESI-MS(+) m/z [M+2H]²⁺: 945.1.

### Preparation of Example 1038

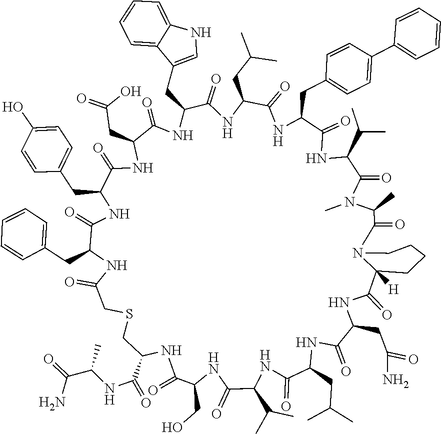

Example 1038 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Single-Coupling Manual Addition Procedure B” was followed with Fmoc-D-Mor-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×150 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 20-70% B over 20 minutes, then a 2-minute hold at 100% B; Flow: 40 mL/min. Fractions containing the desired product were combined and dried via centrifugal evaporation. The yield of the product was 8 mg, and its estimated purity by LCMS analysis was 90%.
Analysis condition A: Retention time=1.64 min; ESI-MS(+) m/z [M+2H]²⁺: 945.1.
Analysis condition B: Retention time=1.81, 1.83 min; ESI-MS(+) m/z [M+2H]²⁺: 944.6, 944.6.

### Preparation of Example 1039

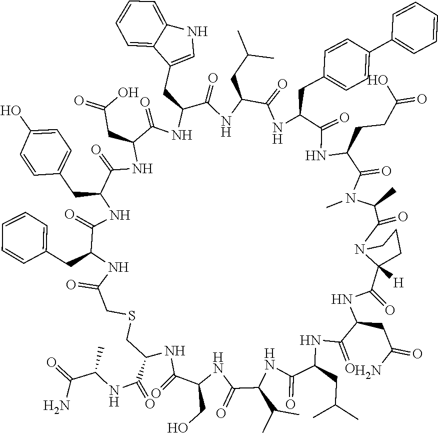

Example 1039 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-55% B over 20 minutes, then a 5-minute hold at 100% B; Flow: 20 mL/min. Fractions containing the desired product were combined and dried via centrifugal evaporation. The yield of the product was 5.4 mg, and its estimated purity by LCMS analysis was 100%.
Analysis condition A: Retention time=1.52 min; ESI-MS(+) m/z [M+H]⁺: 1904.2.
Analysis condition B: Retention time=1.76 min; ESI-MS(+) m/z [M+H]⁺: 1903.9.

### Preparation of Example 1040

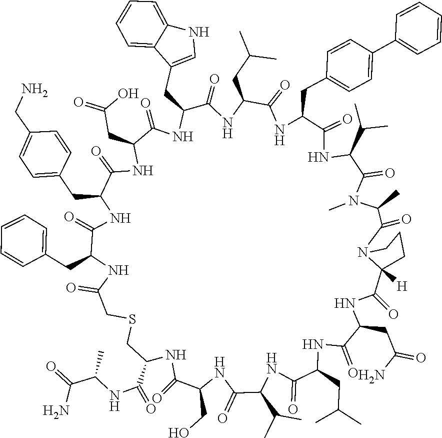

Example 1040 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, Single-Coupling Pre-Activation Procedure” for Fmoc-Phe (4-CH₂NHBoc)-OH, “Symphony Chloroacetic Anhydride coupling procedure”, “Global Deprotection Method A”, and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 20 minutes, then a 5-minute hold at 100% B; Flow: 20 mL/min. Fractions containing the desired product were combined and dried via centrifugal evaporation. The yield of the product was 13.6 mg, and its estimated purity by LCMS analysis was 96.6%.
Analysis condition A: Retention time=1.47 min; ESI-MS(+) m/z [M+2H]²⁺: 944.1.
Analysis condition B: Retention time=1.58 min; ESI-MS(+) m/z [M+2H]²⁺: 944.2.

### Preparation of Example 1041

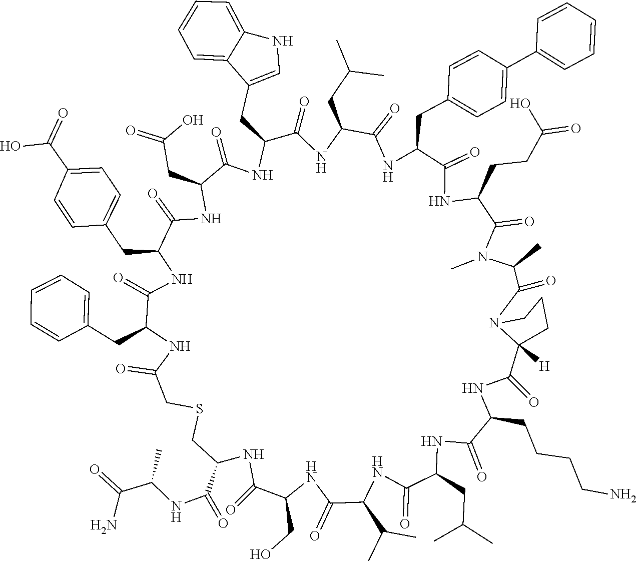

Example 1041 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Single-Coupling Manual Addition Procedure B” was followed with Fmoc-Ala(3-Pyr)-OH and Fmoc-Phe(4-COOtBu)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 12-52% B over 25 minutes, then a 5-minute hold at 100% B; Flow: 20 mL/min. Fractions containing the desired product were combined and dried via centrifugal evaporation. The yield of the product was 23.6 mg, and its estimated purity by LCMS analysis was 89.3%.
Analysis condition A: Retention time=1.4 min; ESI-MS(+) m/z [M+H]⁺: 1948.1.
Analysis condition B: Retention time=1.55 min; ESI-MS(+) m/z [M+H]⁺: 1948.1.

### Preparation of Example 1042

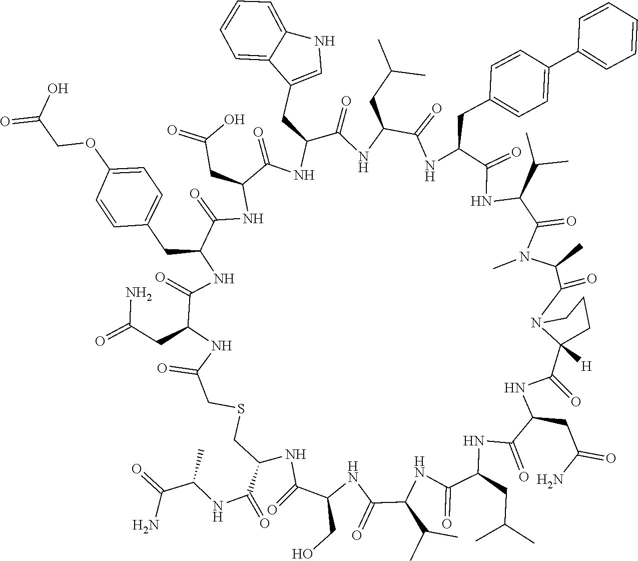

Example 1042 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, “Symphony double-coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 10-50% B over 20 minutes, then a 5-minute hold at 100% B; Flow: 20 mL/min. Fractions containing the desired product were combined and dried via centrifugal evaporation. The yield of the product was 19.7 mg, and its estimated purity by LCMS analysis was 98.9%.
Analysis condition A: Retention time=1.33 min; ESI-MS(+) m/z [M+2H]²⁺: 950.1.
Analysis condition B: Retention time=1.62 min; ESI-MS(+) m/z [M+H]⁺: 1899.2.

### Preparation of Example 1043

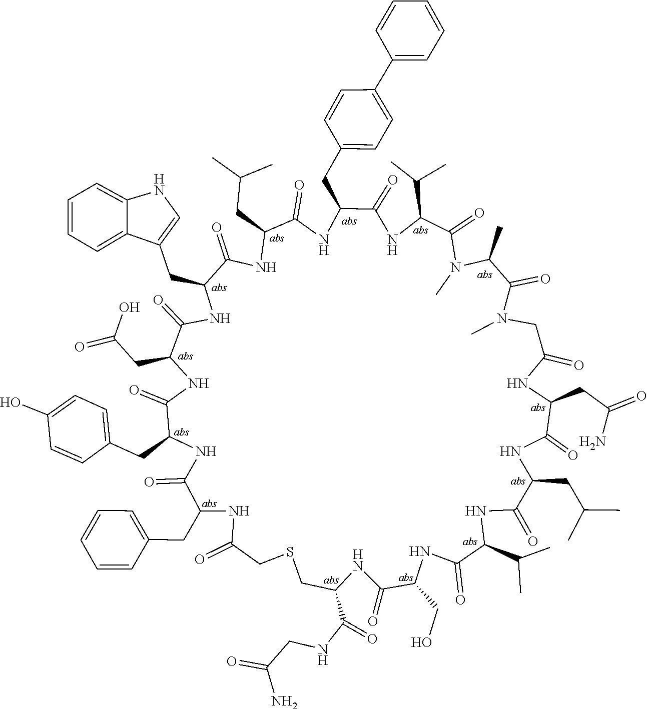 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)C(Cc2ccccc2)NC(=O)CSC[C@@H](C(=O)NCC(N)=O)NC(=O)[C@@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1043 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, “Symphony double-coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 20 minutes, then a 5-minute hold at 100% B; Flow: 20 mL/min. Fractions containing the desired product were combined and dried via centrifugal evaporation. The yield of the product was 25.4 mg, and its estimated purity by LCMS analysis was 94.9%.
Analysis condition A: Retention time=1.58 min; ESI-MS(+) m/z [M+2H]²⁺: 918.2.
Analysis condition B: Retention time=1.73 min; ESI-MS(+) m/z [M+2H]²⁺: 918.2.

### Preparation of Example 1044

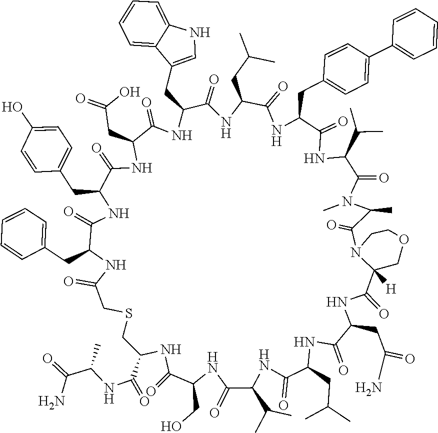 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)[C@H]2COCCN2C(=O)[C@H](C)N(C)C(=O)[C@H](C(C)C)NC(=O)[C@H](Cc2ccc(-c3ccccc3)cc2)NC1=O]

Example 1044 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”,

“Symphony X Single-Coupling Manual Addition Procedure B” was followed with Fmoc-D-Mor-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-65% B over 20 minutes, then a 2-minute hold at 100% B; Flow: 45 mL/min. Fractions containing the desired product were combined and dried via centrifugal evaporation. The yield of the product was 3.5 mg, and its estimated purity by LCMS analysis was 92.3%.
Analysis condition A: Retention time=1.54 min; ESI-MS(+) m/z [M+2H]²⁺: 945.1.
Analysis condition B: Retention time=1.73 min; ESI-MS(+) m/z [M+2H]²⁺: 946.0.

### Preparation of Example 1045

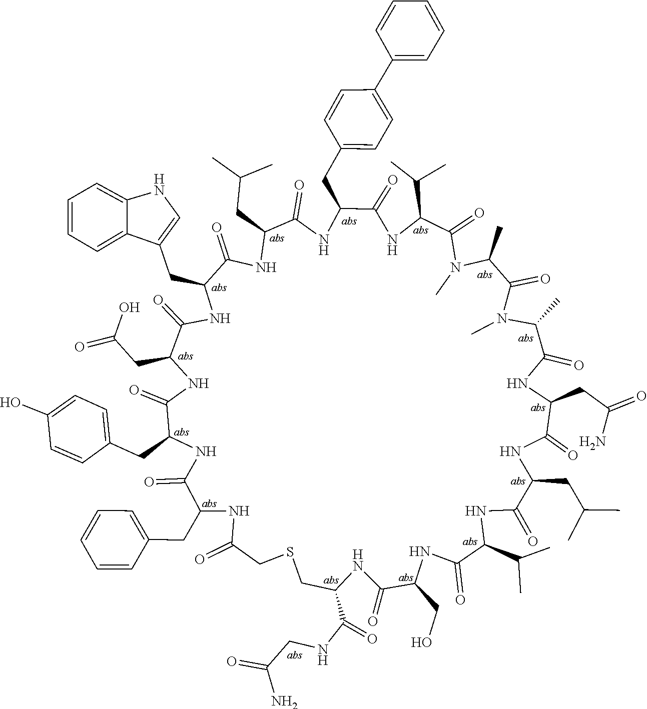 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)C(Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CC(N)=O)NC(=O)[C@@H](C)N(C)C(=O)[C@H](C)N(C)C(=O)[C@H](C(C)C)NC(=O)[C@H](Cc2ccc(-c3ccccc3)cc2)NC1=O]

Example 1045 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, Single-Coupling Pre-Activation Procedure” for Fmoc-NMe-D-Ala-OH, “Symphony Chloroacetic Anhydride coupling procedure”, “Global Deprotection Method A”, and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 19 minutes, then a 5-minute hold at 100% B; Flow: 20 mL/min. Fractions containing the desired product were combined and dried via centrifugal evaporation. The yield of the product was 18.5 mg, and its estimated purity by LCMS analysis was 100%.
Analysis condition A: Retention time=1.72 min; ESI-MS(+) m/z [M+H]⁺: 1863.1.
Analysis condition B: Retention time=1.88 min; ESI-MS(+) m/z [M+2H]²⁺: 932.1.

### Preparation of Example 1046

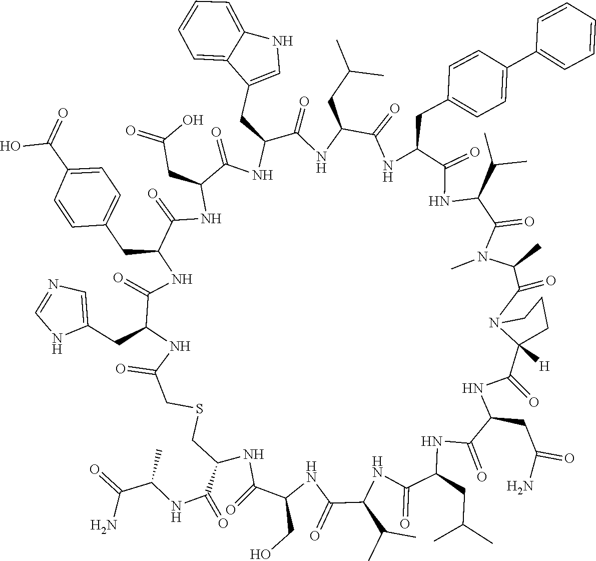

Example 1046 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, “Symphony double-coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 15-55% B over 20 minutes, then a 5-minute hold at 100% B; Flow: 20 mL/min. Fractions containing the desired product were combined and dried via centrifugal evaporation. The yield of the product was 44.6 mg, and its estimated purity by LCMS analysis was 95.3%.
Analysis condition A: Retention time=1.38 min; ESI-MS(+) m/z [M+H]⁺: 1892.2.
Analysis condition B: Retention time=1.59 min; ESI-MS(+) m/z [M+H]⁺: 1893.0.

### Preparation of Example 1047

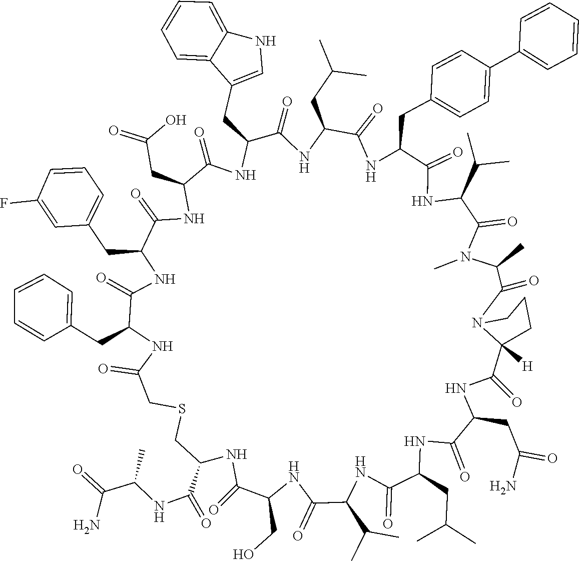

Example 1047 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, Single-Coupling Pre-Activation Procedure” for Fmoc-Phe(3-F)—OH, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-70% B over 20 minutes, then a 4-minute hold at 100% B; Flow: 20 mL/min. Fractions containing the desired product were combined and dried via centrifugal evaporation. The yield of the product was 31.3 mg, and its estimated purity by LCMS analysis was 94.3%.
Analysis condition A: Retention time=1.8 min; ESI-MS(+) m/z [M+H]⁺: 1877.1.
Analysis condition B: Retention time=1.94 min; ESI-MS(+) m/z [M+H]⁺: 1877.2.

### Preparation of Example 1048

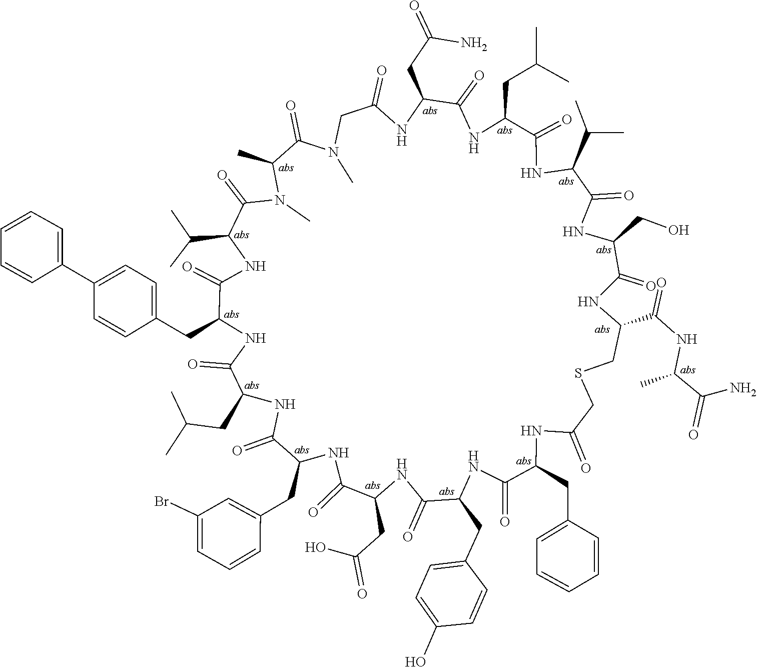 [SMILES: CC(C)C[C@@H]1NC(=O)C[C@H](Cc2cccc(Br)c2)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1048 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, Single-Coupling Pre-Activation Procedure” for Fmoc-Phe (3-Br)—OH, “Symphony Chloroacetic Anhydride coupling procedure”, “Global Deprotection Method A”, and “Cyclization Method”.

The crude material was purified via preparative LC/MS with the following conditions: Column: XBridge C18, 19×200 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 30-70% B over 20 minutes, then a 3-minute hold at 100% B; Flow: 20 mL/min. Fractions containing the desired product were combined and dried via centrifugal evaporation. The yield of the product was 17.6 mg, and its estimated purity by LCMS analysis was 96.8%.
Analysis condition A: Retention time=1.68 min; ESI-MS(+) m/z [M+H]⁺: 1888.0.
Analysis condition B: Retention time=1.91 min; ESI-MS(+) m/z [M+2H]²⁺: 945.3.

### Preparation of Example 1049

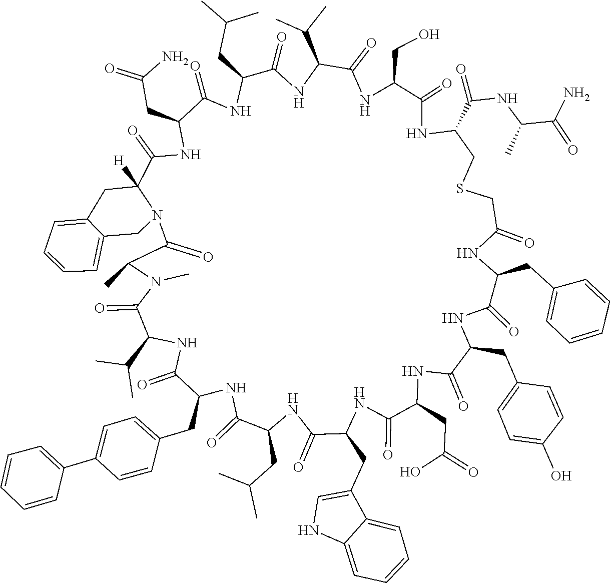 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)[C@H]2Cc3ccccc3CN2C(=O)[C@H](C)N(C)C(=O)[C@H](C(C)C)NC(=O)[C@H](Cc2ccc(-c3ccccc3)cc2)NC1=O]

Example 1049 was prepared, using Sieber or Rink on a −1 μmol scale, following the general synthetic sequence described for the preparation of Example 1000 composed of the following general procedures: “Symphony Resin-swelling procedure”, “Symphony Single-coupling procedure”, Single-Coupling Pre-Activation Procedure” for Fmoc-D-Tic-OH, “Symphony Chloroacetic Anhydride coupling procedure”, “Global Deprotection Method A”, and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 20 minutes, then a 5-minute hold at 100% B; Flow: 20 mL/min. Fractions containing the desired product were combined and dried via centrifugal evaporation. The yield of the product was 17.3 mg, and its estimated purity by LCMS analysis was 97.6%.
Analysis condition A: Retention time=1.79 min; ESI-MS(+) m/z [M+2H]²⁺: 969.0.
Analysis condition B: Retention time=1.9 min; ESI-MS(+) m/z [M+2H]²⁺: 969.0.

### Preparation of Example 1050

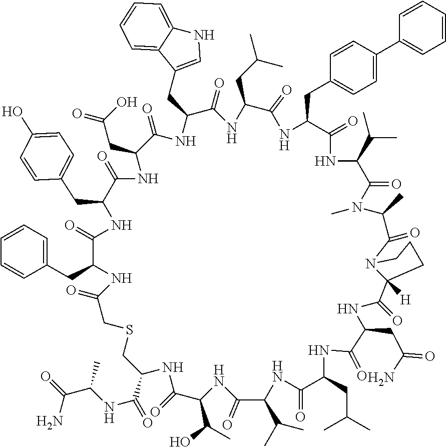

Example 1050 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, “Symphony double-coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 18-58% B over 20 minutes, then a 3-minute hold at 100% B; Flow: 20 mL/min. Fractions containing the desired product were combined and dried via centrifugal evaporation. The yield of the product was 40.3 mg, and its estimated purity by LCMS analysis was 97.5%.

Analysis condition B: Retention time=1.8 min; ESI-MS(+) m/z [M+H]⁺: 1889.0.

### Preparation of Example 1051

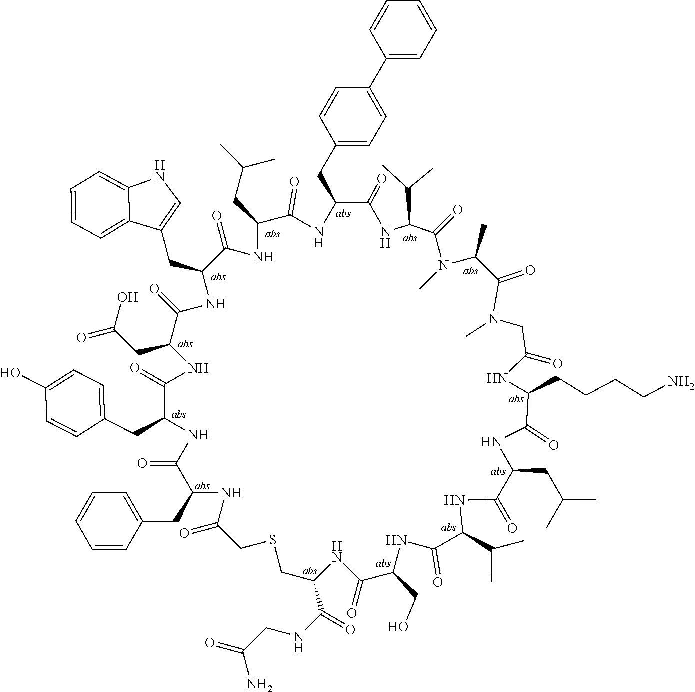 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CCCCN)NC(=O)CN(C)C(=O)[C@H](C)N(C)C(=O)[C@H](C(C)C)NC(=O)[C@H](Cc2ccc(-c3ccccc3)cc2)NC1=O]

Example 1051 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”,

“Symphony X Chloroacetic Anhydride coupling procedure”, “Symphony X Final rinse and dry procedure”, “Global Deprotection Method B”, “Cyclization Method A”.

The crude material was purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 10-60% B over 20 minutes, then a 2-minute hold at 100% B; Flow: 45 mL/min. Fractions containing the desired product were combined and dried via centrifugal evaporation. The yield of the product was 17.2 mg, and its estimated purity by LCMS analysis was 99.2%.
Analysis condition A: Retention time=1.52 min; ESI-MS(+) m/z [M+2H]²⁺: 925.1.
Analysis condition B: Retention time=1.58 min; ESI-MS(+) m/z [M+2H]²⁺: 925.1.

### Preparation of Example 1052

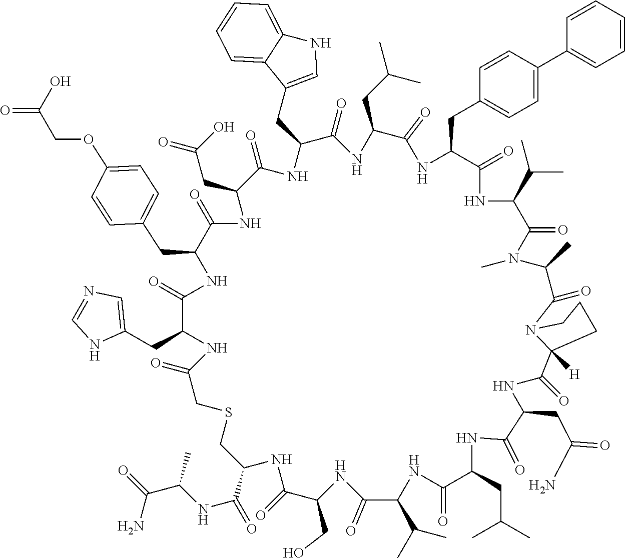

Example 1052 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, “Symphony double-coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 15-55% B over 25 minutes, then a 5-minute hold at 100% B; Flow: 20 mL/min. Fractions containing the desired product were combined and dried via centrifugal evaporation. The yield of the product was 36.2 mg, and its estimated purity by LCMS analysis was 95.4%.
Analysis condition A: Retention time=1.37 min; ESI-MS(+) m/z [M+2H]²⁺: 962.1.
Analysis condition B: Retention time=1.53 min; ESI-MS(+) m/z [M+2H]²⁺: 962.1.

### Preparation of Example 1053

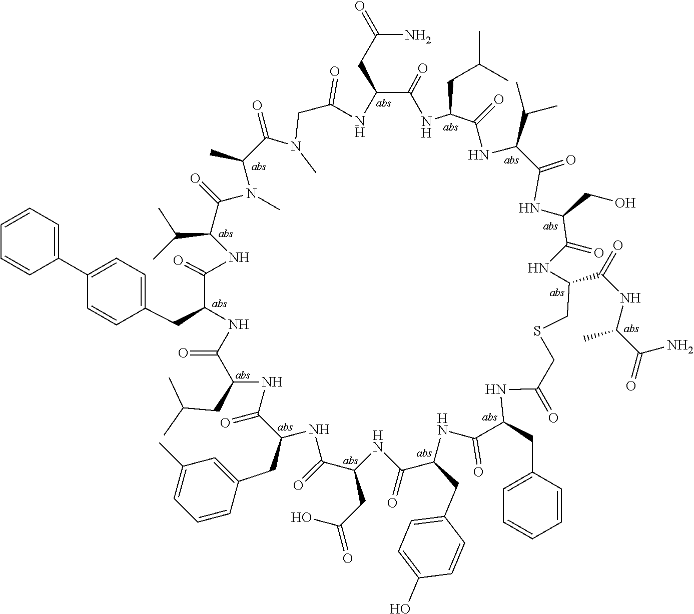 [SMILES: Cc1cccc(C[C@@H]2NC(=O)[C@H](CC(=O)O)NC(=O)[C@H](Cc3ccc(O)cc3)NC(=O)[C@H](Cc3ccccc3)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 1053 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, Single-Coupling Pre-Activation Procedure” for Fmoc-Phe (3-Me)-OH, “Symphony Chloroacetic Anhydride coupling procedure”, “Global Deprotection Method A”, and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 20 minutes, then a 3-minute hold at 100% B; Flow: 20 mL/min. Fractions containing the desired product were combined and dried via centrifugal evaporation. The yield of the product was 26.9 mg, and its estimated purity by LCMS analysis was 92.4%.
Analysis condition A: Retention time=1.64 min; ESI-MS(+) m/z [M+H]⁺: 1823.1.
Analysis condition B: Retention time=1.88 min; ESI-MS(+) m/z [M+H]⁺: 1822.9.

### Preparation of Example 1054

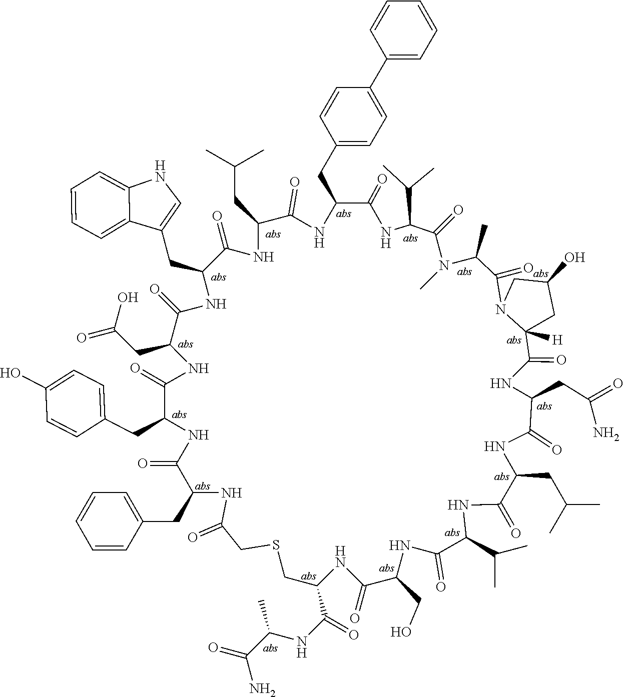 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)[C@H]2C[C@H](O)CN2C(=O)[C@H](C)N(C)C(=O)[C@H](C(C)C)NC(=O)[C@H](Cc2ccc(-c3ccccc3)cc2)NC1=O]

To a 45-mL polypropylene solid-phase reaction vessel was added Sieber resin or Rink resin (428 mg for Sieber or 564 mg for Rink, 0.300 mmol), and the reaction vessel was placed on the Prelude peptide synthesizer. The following procedures were then performed sequentially:

“Prelude Resin-swelling procedure” was followed; “Prelude Single-coupling procedure” was followed with Fmoc-Ala-OH; “Prelude Single-coupling procedure” was followed with Fmoc-Cys(Trt)-OH; “Prelude Single-coupling procedure” was followed with Fmoc-Ser(tBu)-OH; “Prelude Single-coupling procedure” was followed with Fmoc-Val-OH; “Prelude Single-coupling procedure” was followed with Fmoc-Leu-OH; “Prelude Single-coupling procedure” was followed with Fmoc-Asn(Trt)-OH;

The resin was split into 0.050 mmol and was transferred to a different 45-mL polypropylene solid-phase reaction vessel, and the reaction vessel was placed on the Symphony X peptide synthesizer. The following procedures were then performed sequentially:

“Symphony X Single-Coupling Manual Addition Procedure B” was followed with Fmoc-D-Hyp-OH; “Symphony X Single-coupling procedure” was followed with Fmoc-NMe-Ala-OH; “Symphony X Single-coupling procedure” was followed with Fmoc-Val-OH; “Symphony X Single-coupling procedure” was followed with Fmoc-Bip-OH; “Symphony X Single-coupling procedure” was followed with Fmoc-Leu-OH; “Symphony X Single-coupling procedure” was followed with Fmoc-Trp(Boc)-OH; “Symphony X Single-coupling procedure” was followed with Fmoc-Asp(tBu)-OH; “Symphony X Single-coupling procedure” was followed with Tyr(tBu)-OH; “Symphony X Single-coupling procedure” was followed with Fmoc-Phe-OH; “Symphony X Chloroacetic Anhydride coupling procedure” was followed; “Symphony X Final rinse and dry procedure” was followed; “Global Deprotection Method A” was followed; “Cyclization Method A” was followed.

The crude material was purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-65% B over 20 minutes, then a 2-minute hold at 100% B; Flow: 45 mL/min. Fractions containing the desired product were combined and dried via centrifugal evaporation. The yield of the product was 1.7 mg, and its estimated purity by LCMS analysis was 91.7%.
Analysis condition A: Retention time=1.47 min; ESI-MS(+) m/z [M+2H]²⁺: 945.1.
Analysis condition B: Retention time=1.6 min; ESI-MS(+) m/z [M+2H]²⁺: 945.1.

### Preparation of Example 1055

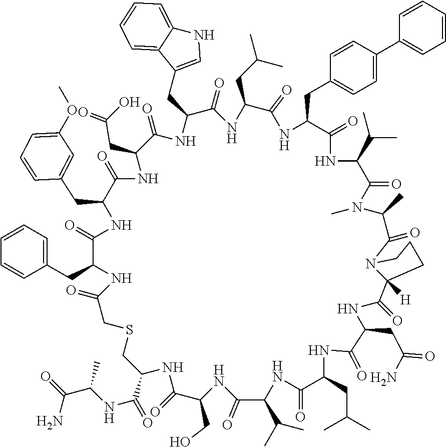

Example 1055 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, Single-Coupling Pre-Activation Procedure” for Fmoc-Phe(3-OMe)-OH, “Symphony Chloroacetic Anhydride coupling procedure”, “Global Deprotection Method A” and “Cyclization Method”.

The crude material was purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-70% B over 20 minutes, then a 2-minute hold at 100% B; Flow: 45 mL/min. Fractions containing the desired product were combined and dried via centrifugal evaporation. The yield of the product was 34.8 mg, and its estimated purity by LCMS analysis was 100%.
Analysis condition A: Retention time=2.06 min; ESI-MS(+) m/z [M+H]⁺: 1887.9.
Analysis condition B: Retention time=1.93 min; ESI-MS(+) m/z [M+H]⁺: 1887.9.

### Preparation of Example 1056

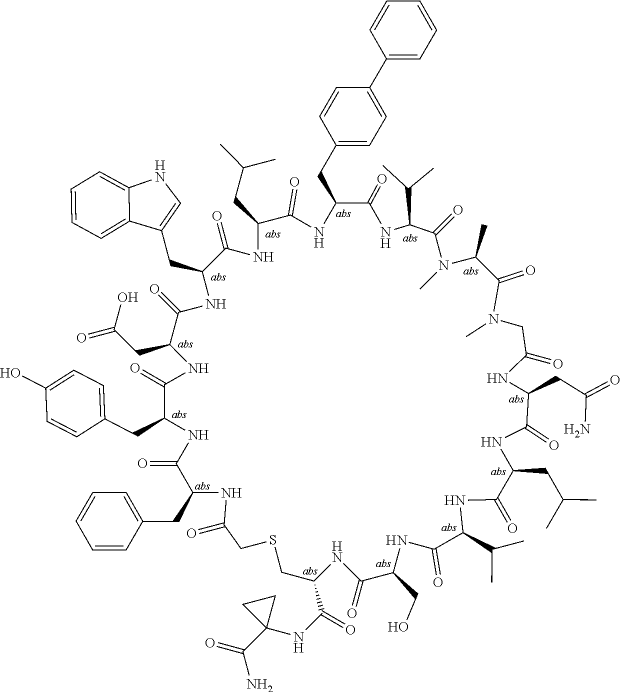 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NC2(C(N)=O)CC2)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1056 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×150 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 15-65% B over 20 minutes, then a 2-minute hold at 100% B; Flow: 40 mL/min. Fractions containing the desired product were combined and dried via centrifugal evaporation. The yield of the product was 35.9 mg, and its estimated purity by LCMS analysis was 96.2%.
Analysis condition A: Retention time=1.62 min; ESI-MS(+) m/z [M+H]⁺: 1860.0.
Analysis condition B: Retention time=1.78 min; ESI-MS(+) m/z [M+H]⁺: 1860.2.

### Preparation of Example 1057

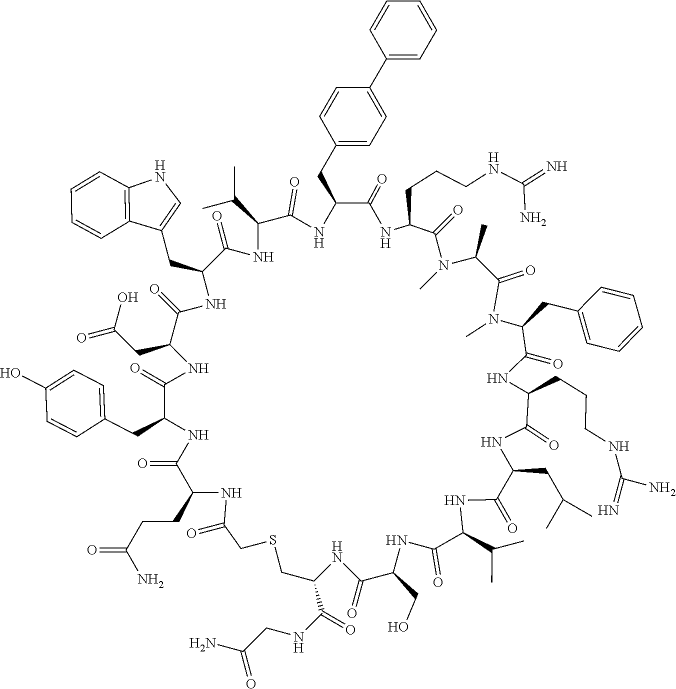 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CCC(N)=O)NC(=O)CSC[C@@H](C(=O)NCC(N)=O)NC(=O)[C@H](CO)NC(=O)[C@H](C(C)C)NC1=O]

Example 1057 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”. “Global Deprotection Method A”, “Cyclization Method A”.

The crude material was purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-65% B over 20 minutes, then a 2-minute hold at 100% B; Flow: 45 mL/min. Fractions containing the desired product were combined and dried via centrifugal evaporation. The yield of the product was 29.5 mg, and its estimated purity by LCMS analysis was 98.2%.
Analysis condition A: Retention time=1.63 min; ESI-MS(+) m/z [M+2H]²⁺:996.1.
Analysis condition B: Retention time=1.61 min; ESI-MS(+) m/z [M+2H]²⁺: 996.1.

### Preparation of Example 1058

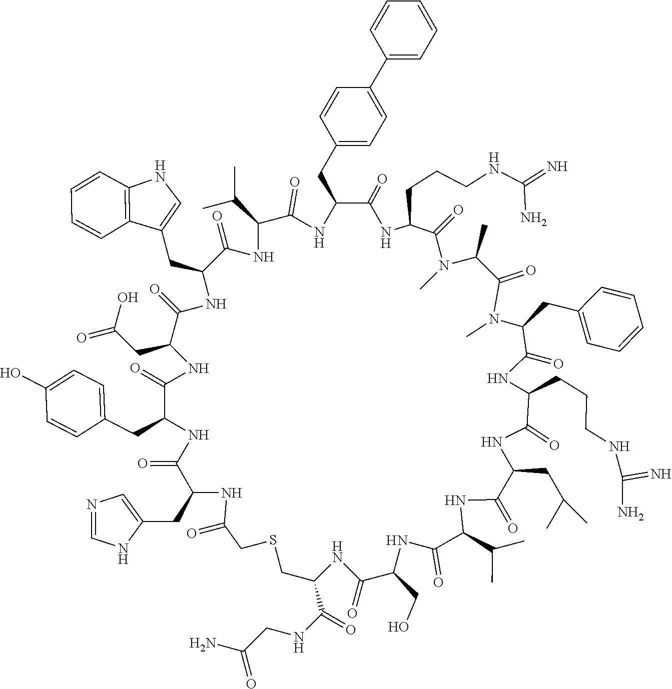 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2cnc[nH]2)NC(=O)CSC[C@@H](C(=O)NCC(N)=O)NC(=O)[C@H](CO)NC(=O)[C@H](C(C)C)NC1=O]

Example 1058 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-65% B over 20 minutes, then a 2-minute hold at 100% B; Flow: 45 mL/min. Fractions containing the desired product were combined and dried via centrifugal evaporation. The yield of the product was 24.7 mg, and its estimated purity by LCMS analysis was 96.3%.
Analysis condition A: Retention time=1.64 min; ESI-MS(+) m/z [M+2H]²⁺: 1000.1.
Analysis condition B: Retention time=1.53 min; ESI-MS(+) m/z [M+3H]³⁺: 667.2.

### Preparation of Example 1059

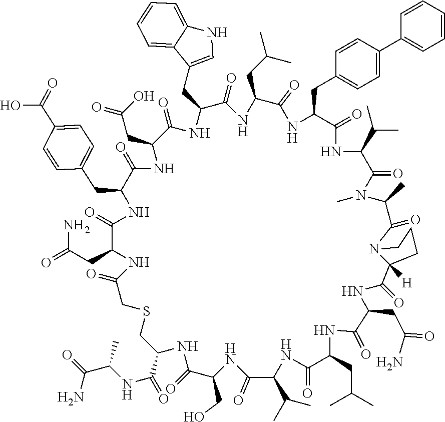

Example 1059 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, “Symphony double-coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 10-50% B over 25 minutes, then a 5-minute hold at 100% B; Flow: 20 mL/min. Fractions containing the desired product were combined and dried via centrifugal evaporation. The yield of the product was 13.9 mg, and its estimated purity by LCMS analysis was 98.7%.
Analysis condition A: Retention time=1.21 min; ESI-MS(+) m/z [M+2H]²⁺: 935.3.
Analysis condition B: Retention time=1.57 min; ESI-MS(+) m/z [M+2H]²⁺: 935.1.

### Preparation of Example 1060

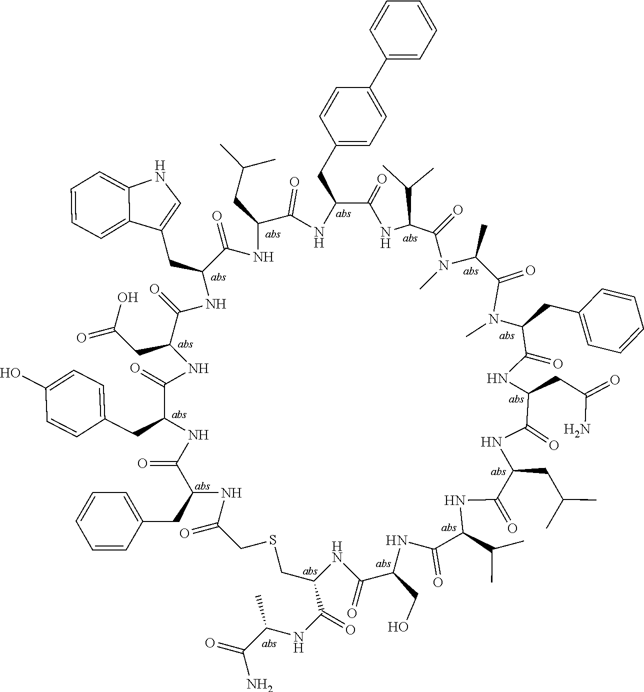 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)[C@H](Cc2ccccc2)N(C)C(=O)[C@H](C)N(C)C(=O)[C@H](C(C)C)NC(=O)[C@H](Cc2ccc(-c3ccccc3)cc2)NC1=O]

Example 1060 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge Phenyl, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 30-70% B over 20 minutes, then a 5-minute hold at 100% B; Flow: 20 mL/min. Fractions containing the desired product were combined and dried via centrifugal evaporation. The material was further purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-70% B over 20 minutes, then a 2-minute hold at 100% B; Flow: 45 mL/min. Fractions containing the desired product were combined and dried via centrifugal evaporation. The yield of the product was 9.7 mg, and its estimated purity by LCMS analysis was 100%.
Analysis condition A: Retention time=1.77 min; ESI-MS(+) m/z [M+H]⁺: 1938.1.
Analysis condition B: Retention time=1.96 min; ESI-MS(+) m/z [M+H]⁺: 1939.2.

### Preparation of Example 1061

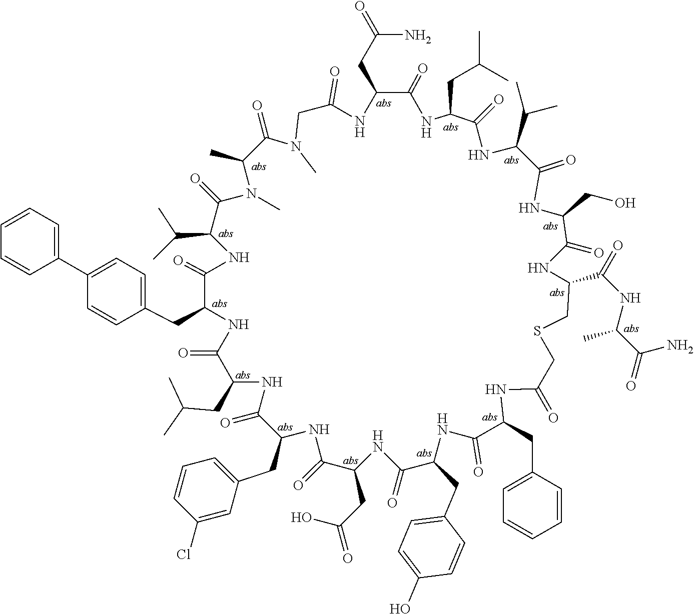 [SMILES: CC(C)C[C@@H]1NC(=O)[C@H](Cc2cccc(Cl)c2)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1061 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Single-Coupling Manual Addition Procedure B” was followed with Fmoc-Phe(3-Cl)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 25 minutes, then a 3-minute hold at 100% B; Flow: 20 mL/min. Fractions containing the desired product were combined and dried via centrifugal evaporation. The yield of the product was 32.6 mg, and its estimated purity by LCMS analysis was 95.5%.
Analysis condition A: Retention time=1.59 min; ESI-MS(+) m/z [M+2H]²⁺: 922.7.
Analysis condition B: Retention time=1.78 min; ESI-MS(+) m/z [M+2H]²⁺: 922.3.

### Preparation of Example 1062

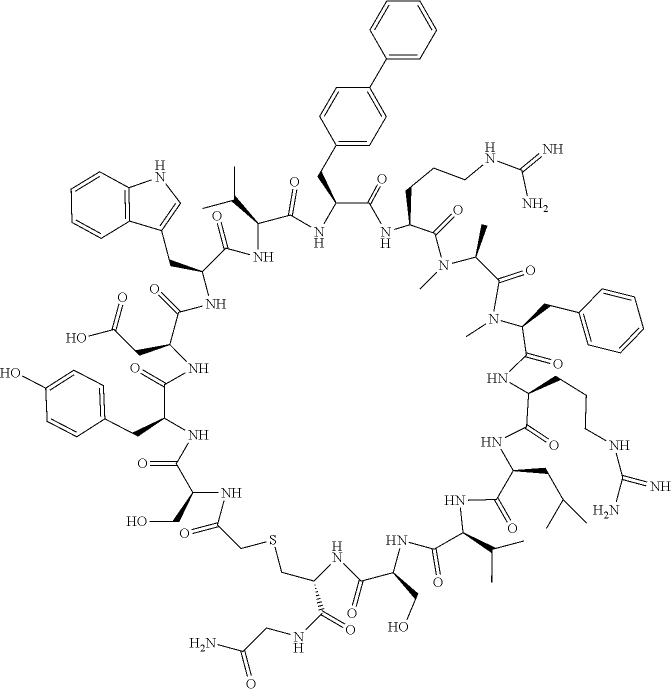 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CO)NC(=O)CSC[C@@H](C(=O)NCC(N)=O)NC(=O)[C@H](CO)NC(=O)[C@H](C(C)C)NC1=O]

Example 1062 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-65% B over 20 minutes, then a 2-minute hold at 100% B; Flow: 45 mL/min. Fractions containing the desired product were combined and dried via centrifugal evaporation. The yield of the product was 25 mg, and its estimated purity by LCMS analysis was 95.1%.
Analysis condition A: Retention time=1.69 min; ESI-MS(+) m/z [M+H]⁺: 1949.0.
Analysis condition B: Retention time=1.68 min; ESI-MS(+) m/z [M+2H]²⁺: 975.2.

### Preparation of Example 1063

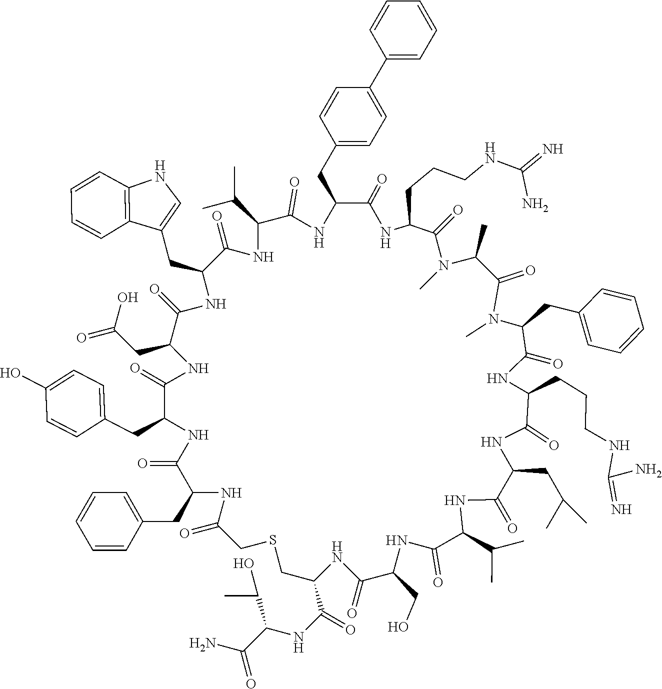 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@H](C(N)=O)C(C)O)NC(=O)[C@H](CO)NC(=O)[C@H](C(C)C)NC1=O]

Example 1063 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”,˜ “Global Deprotection Method A”, “Cyclization Method A”.

The crude material was purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-70% B over 20 minutes, then a 2-minute hold at 100% B; Flow: 45 mL/min. Fractions containing the desired product were combined and dried via centrifugal evaporation. The yield of the product was 21.6 mg, and its estimated purity by LCMS analysis was 87.3%.
Analysis condition A: Retention time=1.79 min; ESI-MS(+) m/z [M−2H]²⁻: 1025.1.
Analysis condition B: Retention time=1.78 min: ESI-MS(+) m/z [M+2H]²⁺: 1027.1.

### Preparation of Example 1064

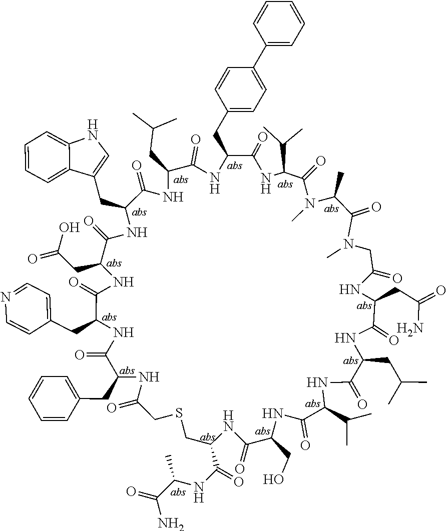 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1064 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”,

“Symphony X Single-Coupling Manual Addition Procedure B” was followed with Fmoc-Ala(3-Pyr)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 20 minutes, then a 4-minute hold at 100% B; Flow: 20 mL/min. Fractions containing the desired product were combined and dried via centrifugal evaporation. The yield of the product was 12.3 mg, and its estimated purity by LCMS analysis was 95.5%.
Analysis condition A: Retention time=1.59 min; ESI-MS(+) m/z [M+H]⁺: 1833.2.
Analysis condition B: Retention time=1.7 min; ESI-MS(+) m/z [M+H]⁺: 1833.1.

### Preparation of Example 1065

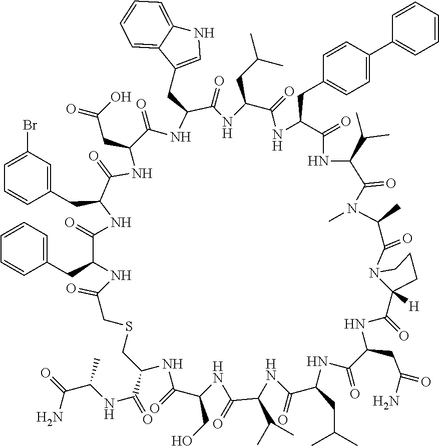

Example 1065 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, Single-Coupling Pre-Activation Procedure” for Fmoc-Phe(3-Br)—OH, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 30-70% B over 20 minutes, then a 4-minute hold at 100% B; Flow: 20 mL/min. Fractions containing the desired product were combined and dried via centrifugal evaporation. The yield of the product was 27.2 mg, and its estimated purity by LCMS analysis was 99%.
Analysis condition A: Retention time=1.83 min; ESI-MS(+) m/z [M+H]⁺: 1936.1.
Analysis condition B: Retention time=2.02 min; ESI-MS(+) m/z [M+H]⁺: 1935.5.

### Preparation of Example 1066

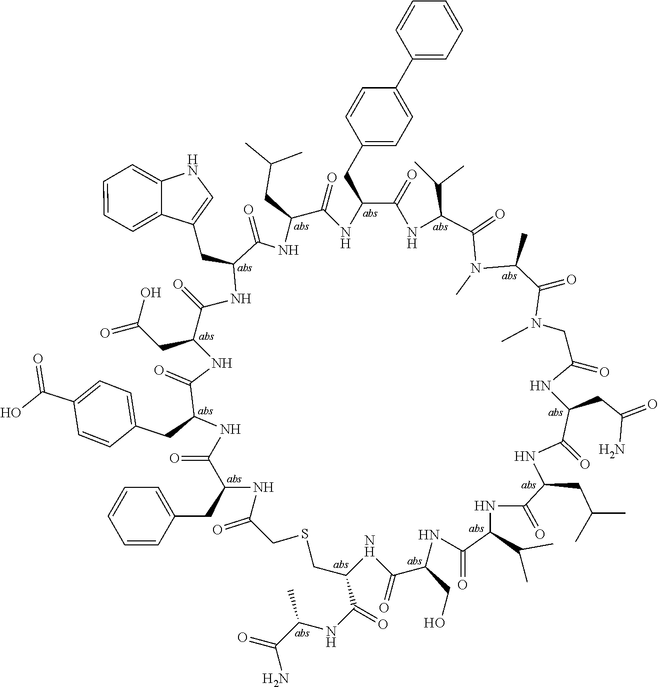 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1066 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×150 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 15-65% B over 20 minutes, then a 2-minute hold at 100% B; Flow: 40 mL/min. Fractions containing the desired product were combined and dried via centrifugal evaporation. The yield of the product was 17.3 mg, and its estimated purity by LCMS analysis was 97.8%.
Analysis condition A: Retention time=1.42 min; ESI-MS(+) m/z [M+H]⁺: 1877.1.
Analysis condition B: Retention time=1.75 min: ESI-MS(+) m/z [M+2H]²⁺: 938.3.

### Preparation of Example 1067

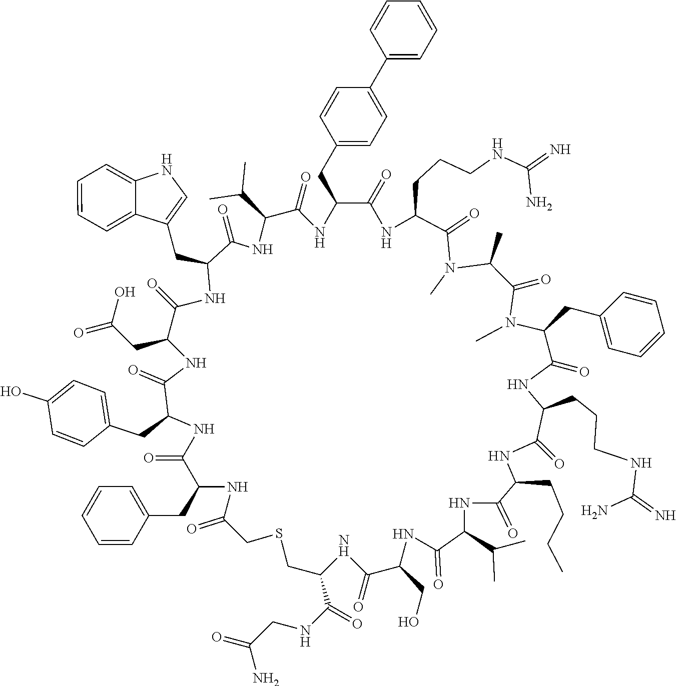 [SMILES: CCCC[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC1=O]

Example 1067 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, “Symphony double-coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 30-70% B over 20 minutes, then a 5-minute hold at 100% B; Flow: 20 mL/min. Fractions containing the desired product were combined and dried via centrifugal evaporation. The yield of the product was 12.1 mg, and its estimated purity by LCMS analysis was 96.7%.
Analysis condition A: Retention time=1.77 min; ESI-MS(+) m/z [M+2H]²⁺: 1005.0.
Analysis condition B: Retention time=1.73 min; ESI-MS(+) m/z [M+2H]²⁺: 1005.3.

### Preparation of Example 1068

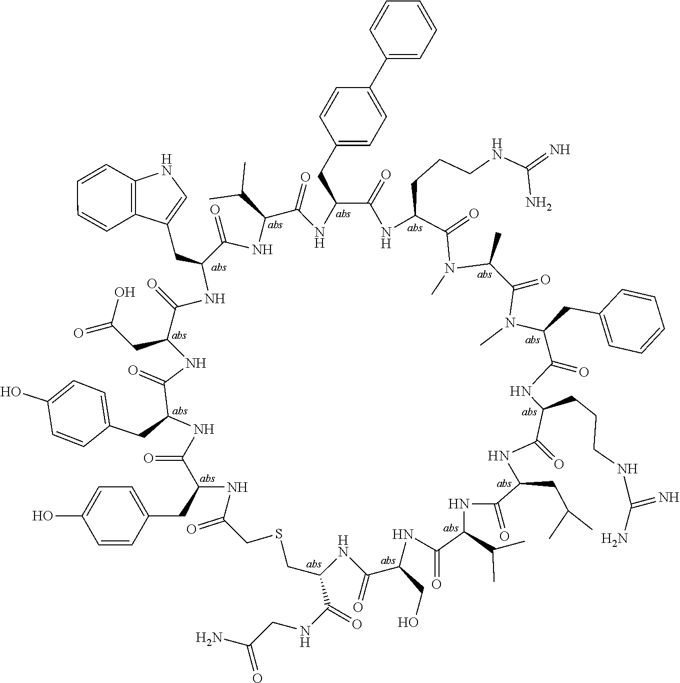 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccc(O)cc2)NC(=O)CSC[C@@H](C(=O)NCC(N)=O)NC(=O)[C@H](CO)NC(=O)[C@H](C(C)C)NC1=O]

Example 1068 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×150 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 15-65% B over 20 minutes, then a 2-minute hold at 100% B; Flow: 40 mL/min. Fractions containing the desired product were combined and dried via centrifugal evaporation. The yield of the product was 34.5 mg, and its estimated purity by LCMS analysis was 94.1%.
Analysis condition A: Retention time=1.72 min; ESI-MS(+) m/z [M+2H]²⁺: 1013.3.
Analysis condition B: Retention time=1.72 min; ESI-MS(+) m/z [M+2H]²⁺: 1013.1.

### Preparation of Example 1069

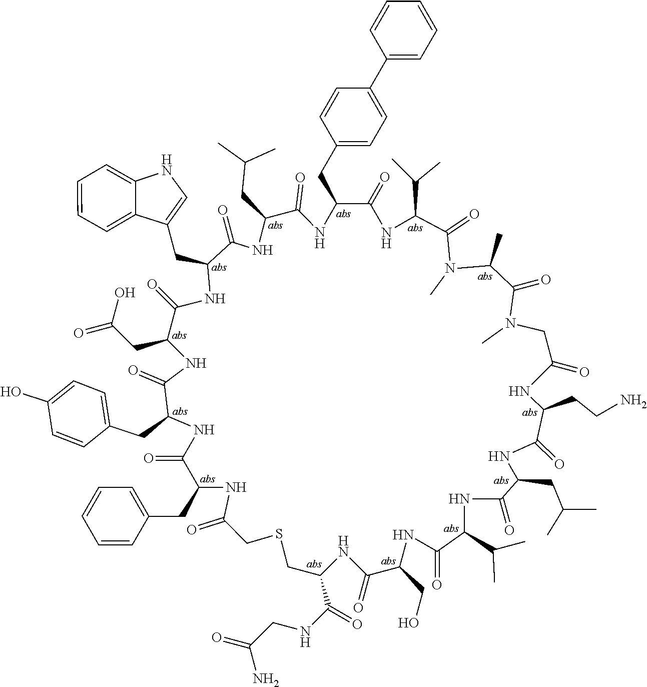 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CCN)NC(=O)CN(C)C(=O)[C@H](C)N(C)C(=O)[C@H](C(C)C)NC(=O)[C@H](Cc2ccc(-c3ccccc3)cc2)NC1=O]

Example 1069 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 10-60% B over 20 minutes, then a 2-minute hold at 100% B; Flow: 45 mL/min. Fractions containing the desired product were combined and dried via centrifugal evaporation. The yield of the product was 28.7 mg, and its estimated purity by LCMS analysis was 92.6%.
Analysis condition A: Retention time=1.61 min; ESI-MS(+) m/z [M+H]⁺: 1819.8.
Analysis condition B: Retention time=1.68 min; ESI-MS(+) m/z [M+H]⁺: 1820.1.

### Preparation of Example 1070

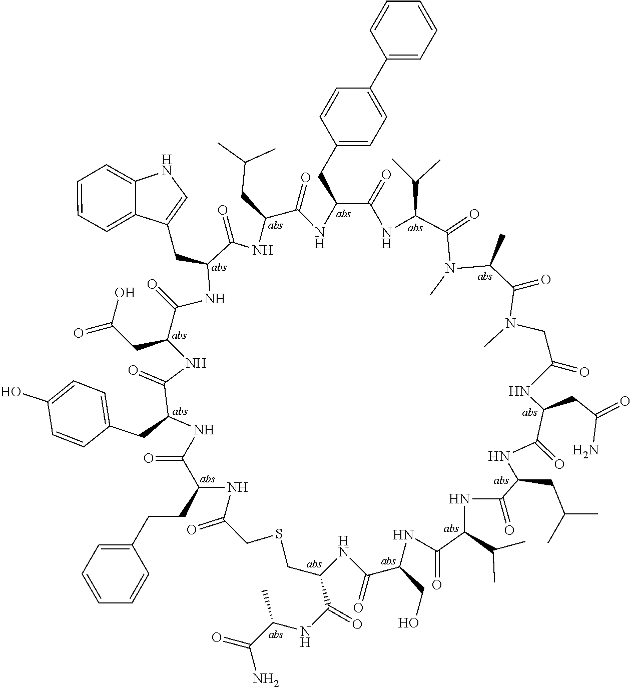 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](CCc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1070 was prepared, using Sieber or Rink on a −1 μmol scale, following the general synthetic sequence described for the preparation of Example 1000 composed of the following general procedures: “Symphony Resin-swelling procedure”, “Symphony Single-coupling procedure”, Single-Coupling Pre-Activation Procedure” for Fmoc-homoPhe-OH, “Symphony Chloroacetic Anhydride coupling procedure”, “Global Deprotection Method A”, and “Cyclization Method”.

The crude material was purified via preparative LC/MS with the following conditions: Column: XBridge C18, 30×200 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 20-70% B over 20 minutes, then a 2-minute hold at 100% B; Flow: 40 mL/min. Fractions containing the desired product were combined and dried via centrifugal evaporation. The yield of the product was 26.4 mg, and its estimated purity by LCMS analysis was 93.5%.
Analysis condition A: Retention time=1.59 min; ESI-MS(+) m/z [M+2H]²⁺: 932.0.
Analysis condition B: Retention time=1.75 min; ESI-MS(+) m/z [M+2H]²⁺: 932.2.

### Preparation of Example 1071

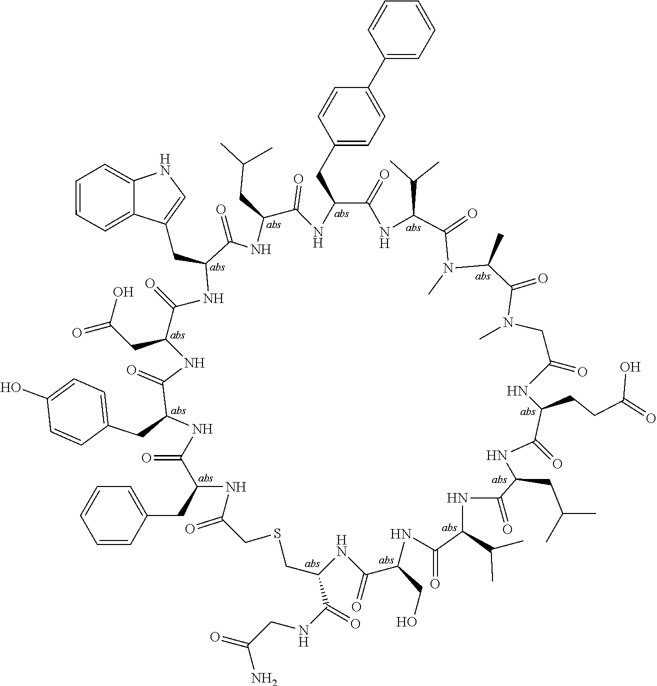 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CCC(=O)O)NC(=O)CN(C)C(=O)[C@H](C)N(C)C(=O)[C@H](C(C)C)NC(=O)[C@H](Cc2ccc(-c3ccccc3)cc2)NC1=O]

Example 1071 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, Symphony X Chloroacetic Anhydride coupling procedure”, “Symphony X Final rinse and dry procedure”, “Global Deprotection Method B”, “Cyclization Method A”.

The crude material was purified via preparative LC/MS with the following conditions: Column: XBridge C18, 30×150 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 15-65% B over 20 minutes, then a 2-minute hold at 100% B; Flow: 40 mL/min. Fractions containing the desired product were combined and dried via centrifugal evaporation. The yield of the product was 20.6 mg, and its estimated purity by LCMS analysis was 97.3%.
Analysis condition A: Retention time=1.41 min; ESI-MS(+) m/z [M+2H]²⁺: 925.4.
Analysis condition B: Retention time=1.68 min; ESI-MS(+) m/z [M+2H]²⁺: 925.1.

### Preparation of Example 1072

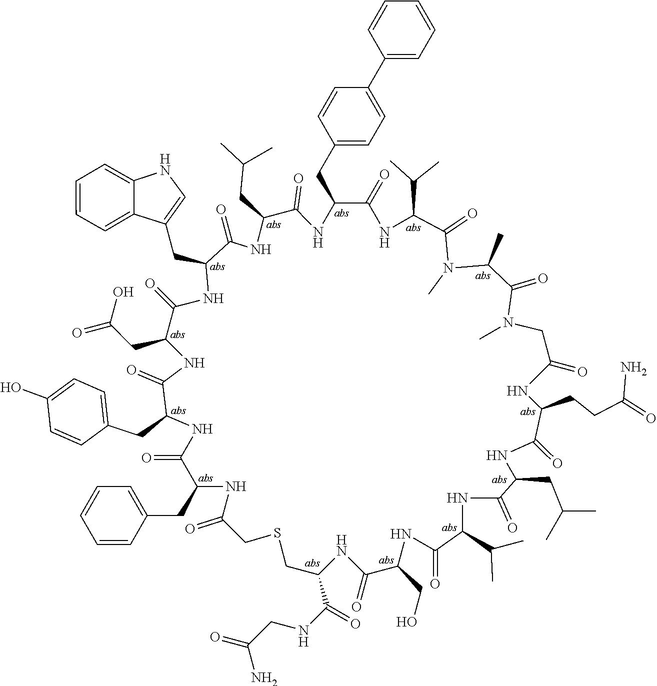 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CCC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1072 was prepared, using Sieber or Kink on a 50 μmol scale, to lowing the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-55% B over 25 minutes, then a 5-minute hold at 100% B; Flow: 20 mL/min. Fractions containing the desired product were combined and dried via centrifugal evaporation. The yield of the product was 33.9 mg, and its estimated purity by LCMS analysis was 95.4%.
Analysis condition A: Retention time=1.58 min; ESI-MS(+) m/z [M+H]⁺: 1847.9.
Analysis condition B: Retention time=1.71 min; ESI-MS(+) m/z [M+H]⁺: 1848.2.

### Preparation of Example 1073

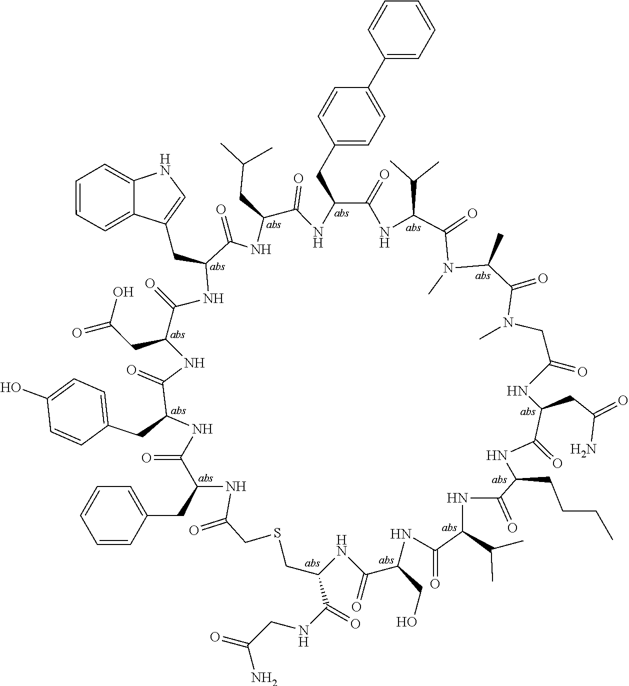 [SMILES: CCCC[C@@H]1NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC1=O]

Example 1073 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×150 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 15-65% B over 20 minutes, then a 2-minute hold at 100% B; Flow: 40 mL/min. Fractions containing the desired product were combined and dried via centrifugal evaporation. The yield of the product was 37.2 mg, and its estimated purity by LCMS analysis was 100%.
Analysis condition A: Retention time=1.6 min; ESI-MS(+) m/z [M+H]⁺: 1835.1.
Analysis condition B: Retention time=1.78 min; ESI-MS(+) m/z [M+H]⁺: 1835.1.

### Preparation of Example 1074

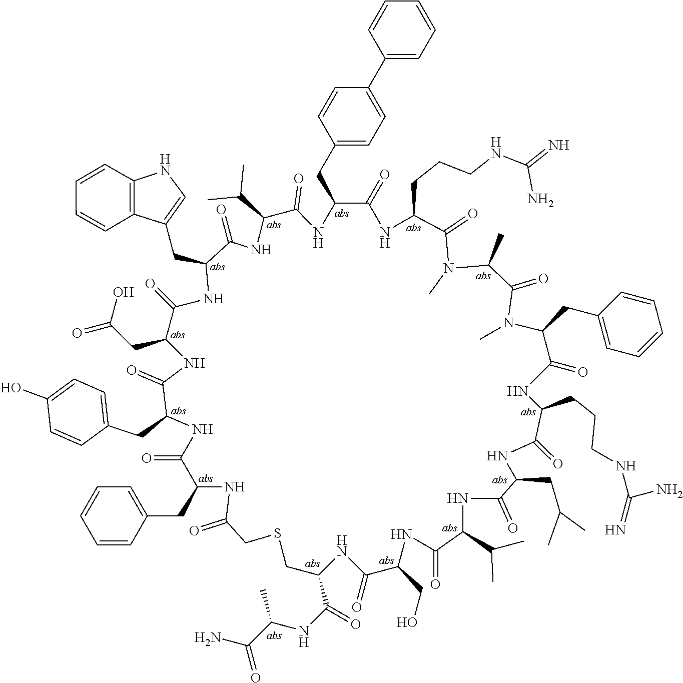 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC1=O]

Example 1074 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×150 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 20-70% B over 20 minutes, then a 2-minute hold at 100% B; Flow: 40 mL/min. Fractions containing the desired product were combined and dried via centrifugal evaporation. The material was further purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-70% B over 20 minutes, then a 2-minute hold at 100% B; Flow: 45 mL/min. Fractions containing the desired product were combined and dried via centrifugal evaporation. The yield of the product was 4.8 mg, and its estimated purity by LCMS analysis was 95.8%.
Analysis condition A: Retention time=1.84 min; ESI-MS(+) m/z [M+2H]²⁺: 1012.0.
Analysis condition B: Retention time=1.81 min; ESI-MS(+) m/z [M+2H]²⁺: 1012.0.

### Preparation of Example 1075

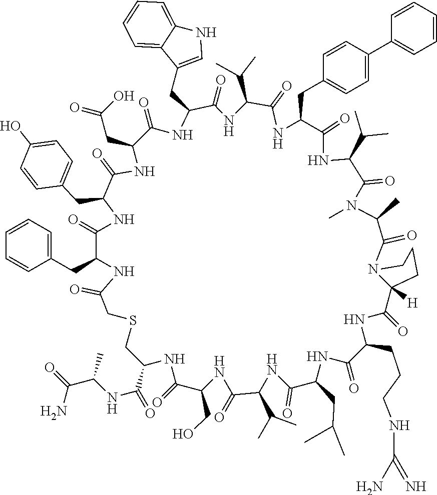

Example 1075 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-55% B over 20 minutes, then a 3-minute hold at 100% B; Flow: 20 mL/min. Fractions containing the desired product were combined and dried via centrifugal evaporation. The yield of the product was 17 mg, and its estimated purity by LCMS analysis was 100%.
Analysis condition A: Retention time=1.75 min; ESI-MS(+) m/z [M+H]⁺: 1903.0.
Analysis condition B: Retention time=1.84 min; ESI-MS(+) m/z [M+H]⁺: 1902.2.

### Preparation of Example 1076

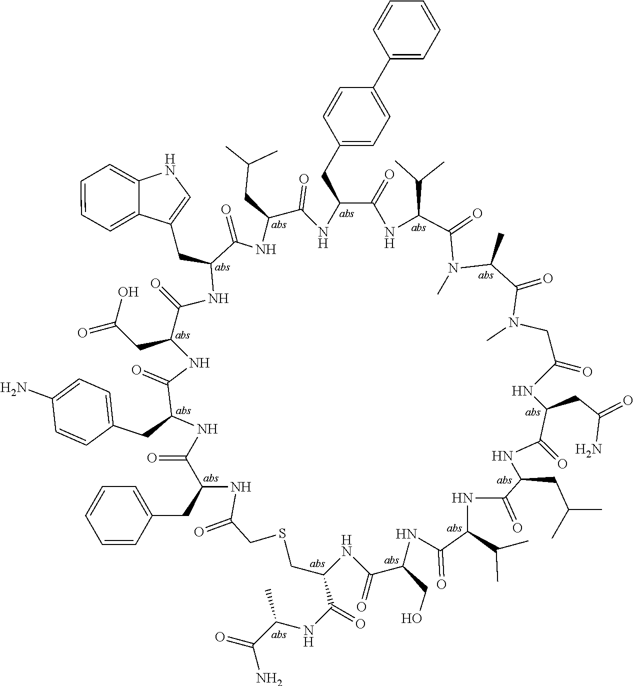 [SMILES: CC(C)C[C@@H]1NC(=O)[C@H](Cc2c[nH]c3ccccc23)NC(=O)[C@H](CC(=O)O)NC(=O)[C@H](Cc2ccc(N)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1076 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”,

“Symphony X Single-Coupling Manual Addition Procedure B” was followed with Fmoc-Phe(4-NHBoc)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 20 minutes, then a 4-minute hold at 100% B; Flow: 20 mL/min. Fractions containing the desired product were combined and dried via centrifugal evaporation. The yield of the product was 30.5 mg, and its estimated purity by LCMS analysis was 97.6%.
Analysis condition A: Retention time=1.54 min; ESI-MS(+) m/z [M+2H]²⁺: 924.0.
Analysis condition B: Retention time=1.58 min; ESI-MS(+) m/z [M+2H]²⁺: 924.4.

### Preparation of Example 1077

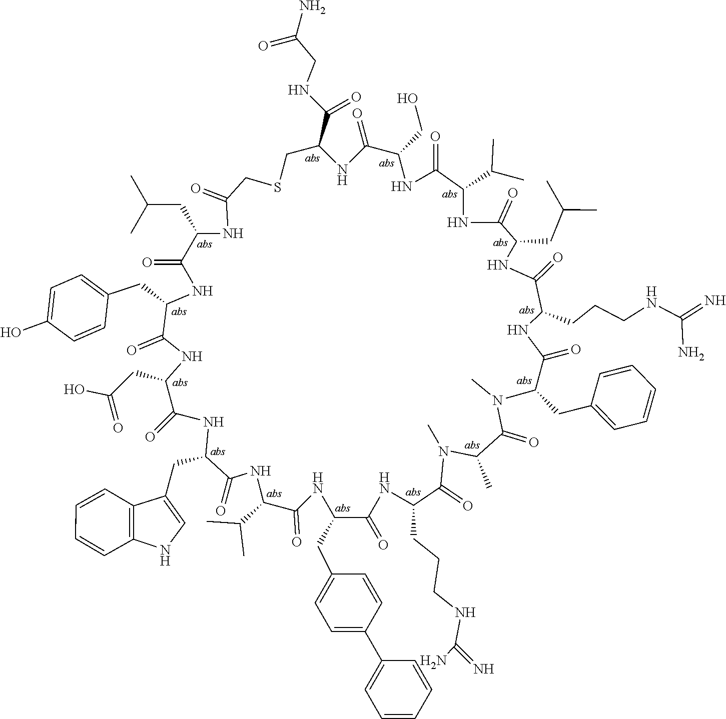 [SMILES: CC(C)C[C@@H]1NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC1=O]

Example 1077 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×150 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 20-70% B over 20 minutes, then a 2-minute hold at 100% B; Flow: 40 mL/min. Fractions containing the desired product were combined and dried via centrifugal evaporation. The yield of the product was 34.5 mg, and its estimated purity by LCMS analysis was 95.5%.
Analysis condition A: Retention time=1.77 min; ESI-MS(+) m/z [M+2H]²⁺: 988.0.
Analysis condition B: Retention time=1.76 min; ESI-MS(+) m/z [M+2H]²⁺: 988.1.

### Preparation of Example 1078

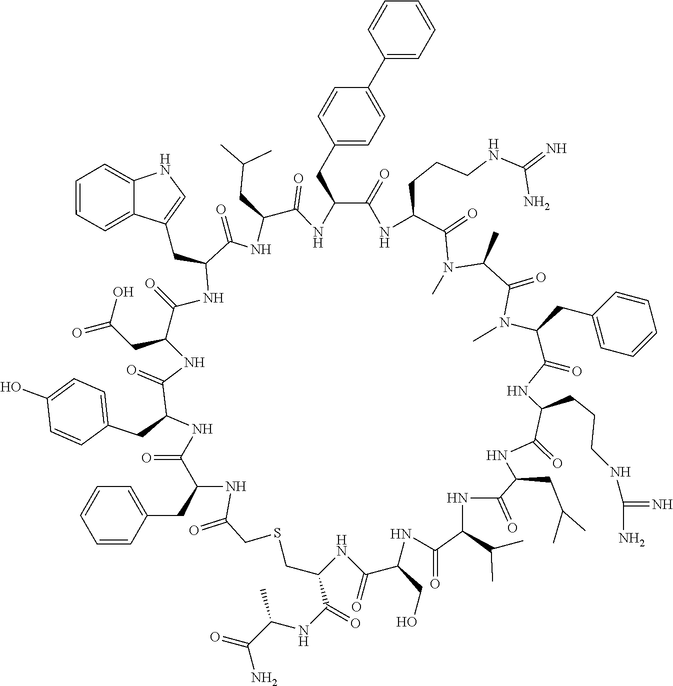 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC1=O]

Example 1078 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×200 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 15-65% B over 20 minutes, then a 2-minute hold at 100% B; Flow: 45 mL/min. Fractions containing the desired product were combined and dried via centrifugal evaporation. The material was further purified via preparative LC/MS with the following conditions: Column: XBridge C18, 30×200 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 15-65% B over 20 minutes, then a 2-minute hold at 100% B; Flow: 45 mL/min. Fractions containing the desired product were combined and dried via centrifugal evaporation. The yield of the product was 4.7 mg, and its estimated purity by LCMS analysis was 100%.
Analysis condition A: Retention time=1.77 min; ESI-MS(+) m/z [M−2H]²⁻: 1017.2.

### Preparation of Example 1079

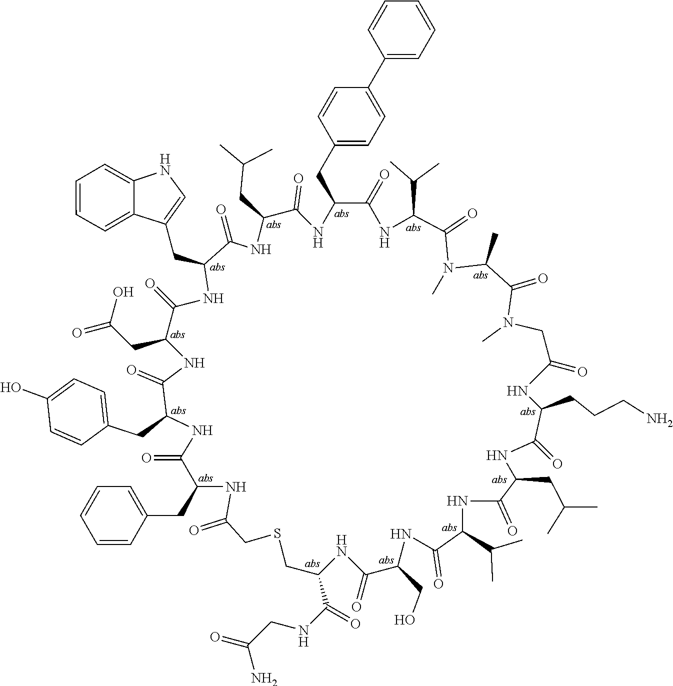 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CCCN)NC(=O)CN(C)C(=O)[C@H](C)N(C)C(=O)[C@H](C(C)C)NC(=O)[C@H](Cc2ccc(-c3ccccc3)cc2)NC1=O]

Example 1079 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 17-57% B over 20 minutes, then a 3-minute hold at 100% B; Flow: 20 mL/min. Fractions containing the desired product were combined and dried via centrifugal evaporation. The yield of the product was 26.8 mg, and its estimated purity by LCMS analysis was 94.1%.
Analysis condition A: Retention time=1.6 min; ESI-MS(+) m/z [M+H]⁺: 1834.1.
Analysis condition B: Retention time=1.67 min; ESI-MS(+) m/z [M+H]⁺: 1834.1.

### Preparation of Example 1080

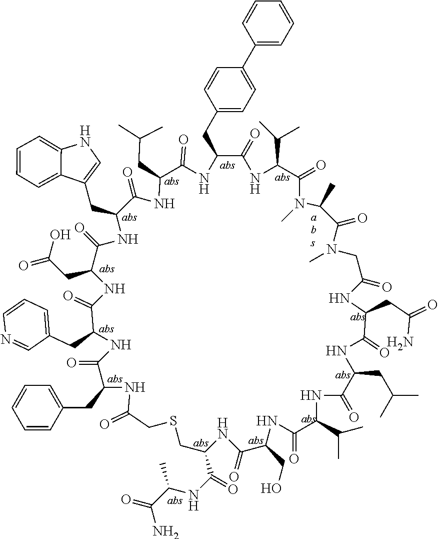 [SMILES: CC(C)C[C@@H]1NC(=O)[C@H](Cc2c[nH]c3ccccc23)NC(=O)[C@H](CC(=O)O)NC(=O)[C@H](Cc2cccnc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1080 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”,

“Symphony X Single-Coupling Manual Addition Procedure B” was followed with Fmoc-Ala(3-Pyr)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 20 minutes, then a 4-minute hold at 100% B; Flow: 20 mL/min. Fractions containing the desired product were combined and dried via centrifugal evaporation. The yield of the product was 13 mg, and its estimated purity by LCMS analysis was 98.1%.
Analysis condition A: Retention time=1.61 min; ESI-MS(+) m/z [M+H]⁺: 1833.0.
Analysis condition B: Retention time=1.71 min; ESI-MS(+) m/z [M+H]⁺: 1833.2.

### Preparation of Example 1081

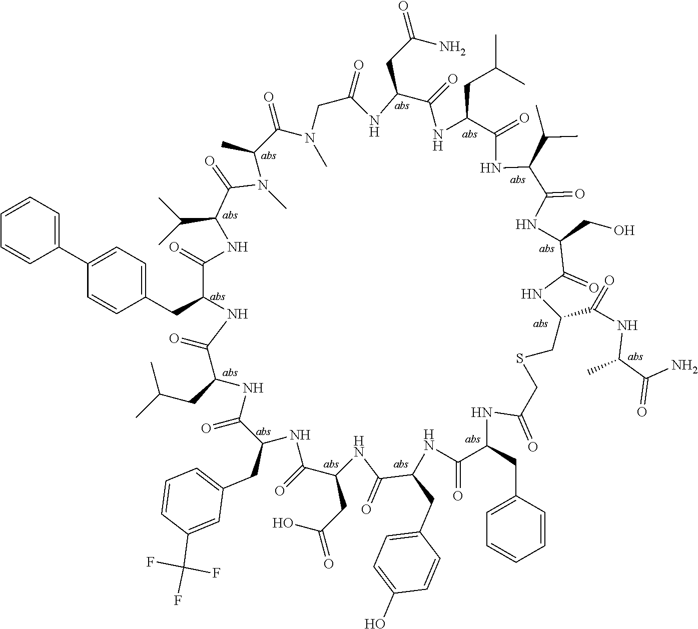 [SMILES: CC(C)C[C@@H]1NC(=O)[C@H](Cc2cccc(C(F)(F)F)c2)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1081 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Single-Coupling Manual Addition Procedure B” was followed with Fmoc-Phe(3-CF₃)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 25-65% B over 25 minutes, then a 5-minute hold at 100% B; Flow: 20 mL/min. Fractions containing the desired product were combined and dried via centrifugal evaporation. The yield of the product was 22 mg, and its estimated purity by LCMS analysis was 98.7%.
Analysis condition A: Retention time=1.77 min; ESI-MS(+) m/z [M+H]⁺: 1877.2.
Analysis condition B: Retention time=1.88 min; ESI-MS(+) m/z [M+H]⁺: 1878.1.

### Preparation of Example 1082

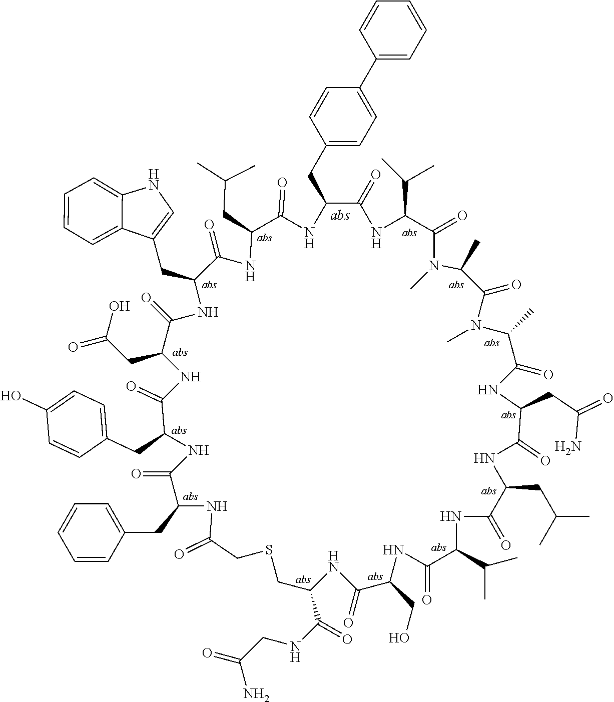 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CC(N)=O)NC(=O)[C@@H](C)N(C)C(=O)[C@H](C)N(C)C(=O)[C@H](C(C)C)NC(=O)[C@H](Cc2ccc(-c3ccccc3)cc2)NC1=O]

Example 1082 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, Single-Coupling Pre-Activation Procedure” for Fmoc-NMe-D-Ala-OH, “Symphony Chloroacetic Anhydride coupling procedure”, “Global Deprotection Method A”, and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 19 minutes, then a 5-minute hold at 100% B; Flow: 20 mL/min. Fractions containing the desired product were combined and dried via centrifugal evaporation. The yield of the product was 22.3 mg, and its estimated purity by LCMS analysis was 100%.
Analysis condition A: Retention time=1.71 min; ESI-MS(+) m/z [M+2H]²⁺: 925.1.
Analysis condition B: Retention time=1.86, min; ESI-MS(+) m/z [M+H]⁺: 1849.3.

### Preparation of Example 1083

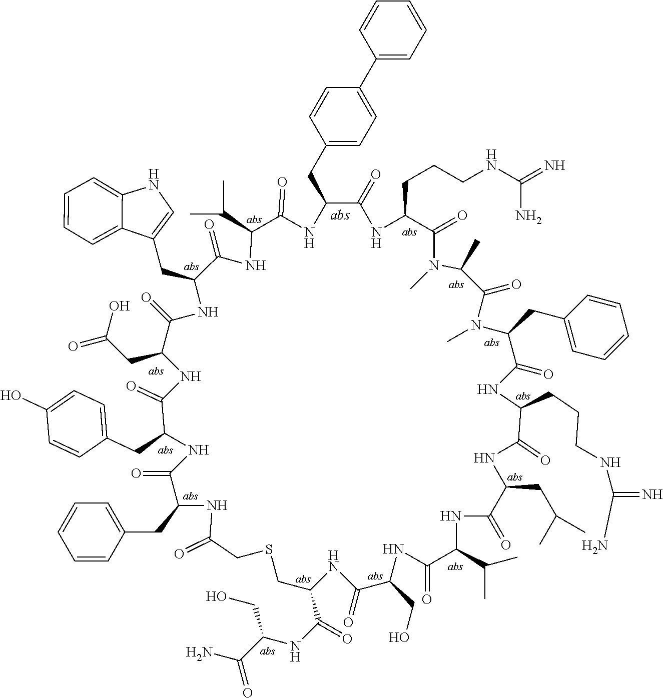 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](CO)C(N)=O)NC(=O)[C@H](CO)NC(=O)[C@H](C(C)C)NC1=O]

Example 1083 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×150 mm, 5-μm particles; MobilePhase A: 5:95 acetonitrile: water with 0.1% trifluoroacetic acid; Mobile Phase B: 95:5 acetonitrile: water with 0.1% trifluoroacetic acid; Gradient: 20-70% B over 20 minutes, then a 2-minute hold at 100% B; Flow: 40 mL/min. Fractions containing the desired product were combined and dried via centrifugal evaporation. The material was further purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-65% B over 20 minutes, then a 2-minute hold at 100% B; Flow: 45 mL/min. Fractions containing the desired product were combined and dried via centrifugal evaporation. The yield of the product was 4.6 mg, and its estimated purity by LCMS analysis was 98.2%.
Analysis condition A: Retention time=1.8 min; ESI-MS(+) m/z [M+2H]²⁺: 1021.1.
Analysis condition B: Retention time=1.79 min; ESI-MS(+) m/z [M+2H]²⁺: 1021.1.

### Preparation of Example 1084

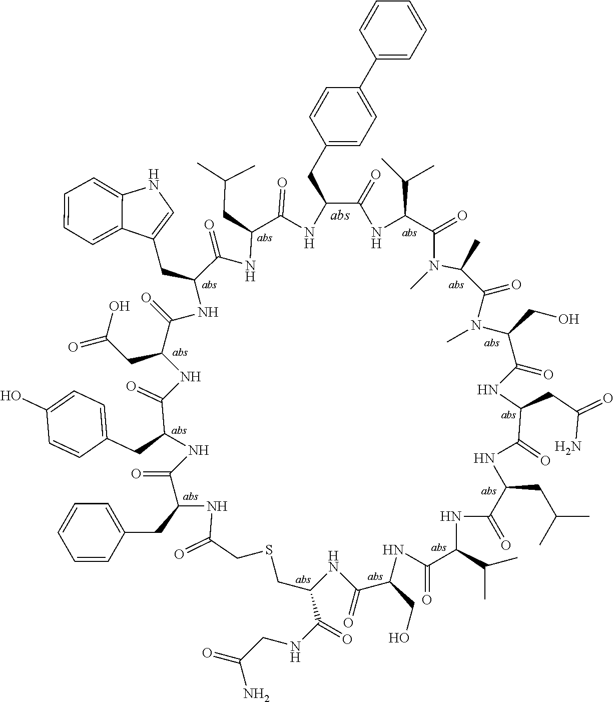 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CC(N)=O)NC(=O)[C@H](CO)N(C)C(=O)[C@H](C)N(C)C(=O)[C@H](C(C)C)NC(=O)[C@H](Cc2ccc(-c3ccccc3)cc2)NC1=O]

Example 1084 was prepared, using Sieber or Rink on a 50 μmol scale, following the general synthetic sequence described for the preparation of Example 1054, composed of the following general procedures: “Prelude Resin-swelling procedure”, “Prelude Single-coupling procedure”, “Symphony Resin-swelling procedure”, “Symphony Single-coupling procedure”, “Symphony Single-Coupling Pre-activation Procedure” was followed with Fmoc-NMe-Ser(tBu)-OH; “Symphony Chloroacetic Anhydride coupling procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 20 minutes, then a 5-minute hold at 100% B; Flow: 20 mL/min. Fractions containing the desired product were combined and dried via centrifugal evaporation. The yield of the product was 15.1 mg, and its estimated purity by LCMS analysis was 96.5%.
Analysis condition A: Retention time=1.54 min; ESI-MS(+) m/z [M+2H]²⁺: 933.0.
Analysis condition B: Retention time=1.72 min; ESI-MS(+) m/z [M+2H]²⁺: 933.2.

### Preparation of Example 1085

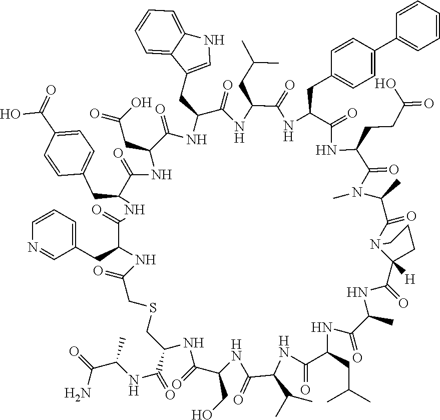

Example 1085 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Single-Coupling Manual Addition Procedure B” was followed with Fmoc-Ala(3-Pyr)-OH and Fmoc-Phe(4-COOtBu)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 7-47% B over 25 minutes, then a 5-minute hold at 100% B; Flow: 20 mL/min. Fractions containing the desired product were combined and dried via centrifugal evaporation. The yield of the product was 33.5 mg, and its estimated purity by LCMS analysis was 99.4%.
Analysis condition A: Retention time=1.21 min; ESI-MS(+) m/z [M+2H]²⁺: 946.0.
Analysis condition B: Retention time=1.68 min; ESI-MS(+) m/z [M+2H]²⁺: 946.0.

### Preparation of Example 1086B

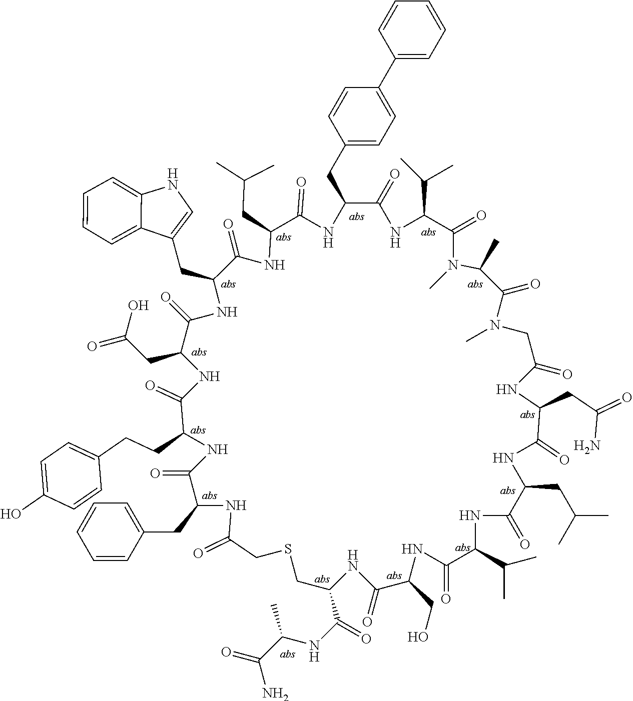 [SMILES: CC(C)C[C@@H]1NC(=O)[C@H](Cc2c[nH]c3ccccc23)NC(=O)[C@H](CC(=O)O)NC(=O)[C@H](CCc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1086 was prepared, using Sieber or Rink on a 50 μmol scale, following the general synthetic sequence described for the preparation of Example 1054, composed of the following general procedures: “Prelude Resin-swelling procedure”, “Prelude Single-coupling procedure”, “Symphony X Resin-swelling procedure”, “Symphony X Single-coupling procedure”, “Symphony X Single-Coupling Manual Addition Procedure B” was followed with Fmoc-Homo-Tyr(tBu)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 30-70% B over 19 minutes, then a 5-minute hold at 100% B; Flow: 20 mL/min. Fractions containing the desired product were combined and dried via centrifugal evaporation. The material was further purified via preparative LC/MS with the following conditions: Column: XBridge C18, 19×150 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 15-65% B over 20 minutes, then a 2-minute hold at 100% B; Flow: 40 mL/min. Fractions containing the desired product were combined and dried via centrifugal evaporation. The yield of the product was 0.9 mg, and its estimated purity by LCMS analysis was 92.5%.
Analysis condition A: Retention time=1.6 min; ESI-MS(+) m/z [M+2H]²⁺: 931.8.

### Preparation of Example 1087

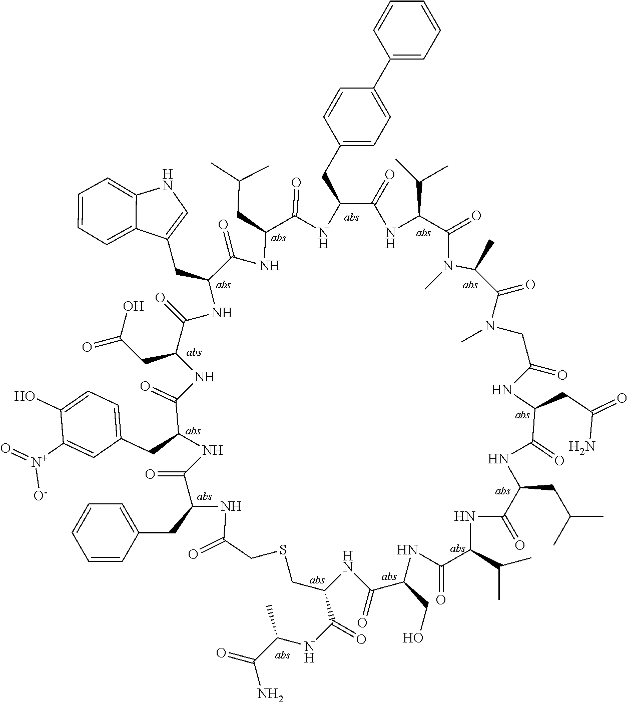 [SMILES: CC(C)C[C@@H]1NC(=O)[C@H](Cc2c[nH]c3ccccc23)NC(=O)[C@H](CC(=O)O)NC(=O)[C@H](Cc2ccc(O)c([N+](=O)[O-])c2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1087 was prepared, using Sieber or Rink on a 50 μmol scale, following the general synthetic sequence described for the preparation of Example 1054, composed of the following general procedures: “Prelude Resin-swelling procedure”, “Prelude Single-coupling procedure”, “Symphony X Resin-swelling procedure”, “Symphony X Single-coupling procedure”, “Symphony X Single-Coupling Manual Addition Procedure B” was followed with Fmoc-Tyr(tBu, 3-NO2)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-70% B over 20 minutes, then a 2-minute hold at 100% B; Flow: 45 mL/min. Fractions containing the desired product were combined and dried via centrifugal evaporation. The yield of the product was 6.4 mg, and its estimated purity by LCMS analysis was 95.1%.
Analysis condition A: Retention time=1.68 min; ESI-MS(+) m/z [M+2H]²⁺: 947.2.
Analysis condition B: Retention time=1.84 min; ESI-MS(+) m/z [M+2H]²⁺: 947.2.

### Preparation of Example 1088

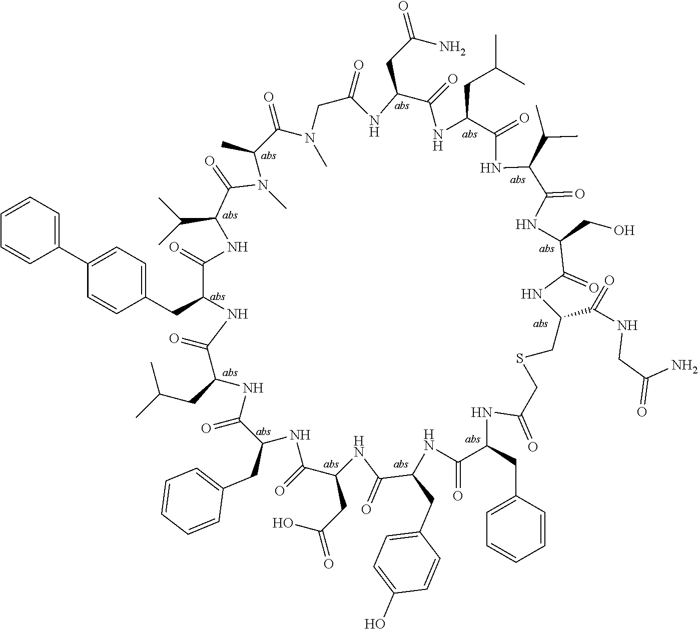 [SMILES: CC(C)C[C@@H]1NC(=O)[C@H](Cc2ccccc2)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1088 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-65% B over 20 minutes, then a 2-minute hold at 100% B; Flow: 45 mL/min. Fractions containing the desired product were combined and dried via centrifugal evaporation. The yield of the product was 33.1 mg, and its estimated purity by LCMS analysis was 100%.
Analysis condition A: Retention time=1.47, 1.52 min; ESI-MS(+) m/z [M+2H]²: 898.26, 898.26.

Analysis condition B: Retention time=1.69 min; ESI-MS(+) m/z [M+2H]²⁺: 898.3.

### Preparation of Example 1089

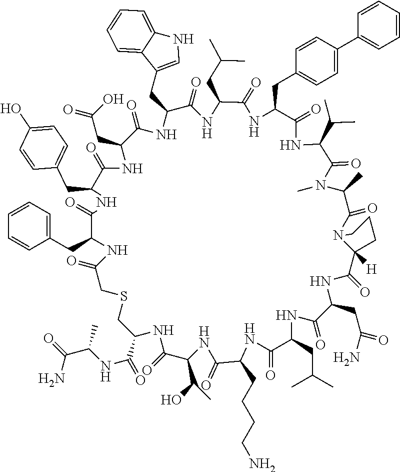

Example 1089 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 20-60% B over 30 minutes, then a 5-minute hold at 100% B; Flow: 20 mL/min. Fractions containing the desired product were combined and dried via centrifugal evaporation. The yield of the product was 12.6 mg, and its estimated purity by LCMS analysis was 86.4%.
Analysis condition A: Retention time=1.74 min; ESI-MS(+) m/z [M+2H]²⁺: 959.9.
Analysis condition B: Retention time=1.78 min; ESI-MS(+) m/z [M+H]⁺: 1917.0.

### Preparation of Example 1090

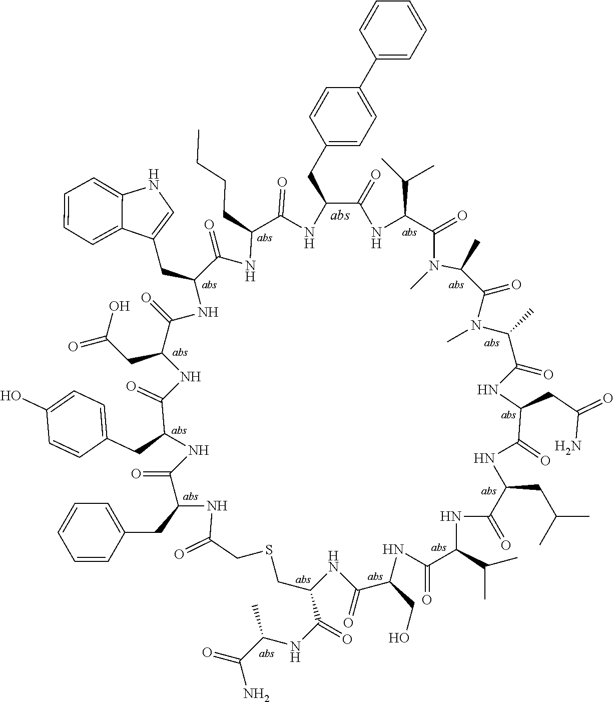 [SMILES: CCCC[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)[C@@H](C)N(C)C(=O)[C@H](C)N(C)C(=O)[C@H](C(C)C)NC(=O)[C@H](Cc2ccc(-c3ccccc3)cc2)NC1=O]

Example 1090 was prepared, using Sieber or Rink on a 50 μmol scale, following the general synthetic sequence described for the preparation of Example 1054, composed of the following general procedures: “Prelude Resin-swelling procedure”, “Prelude Single-coupling procedure”, “Symphony X Resin-swelling procedure”, “Symphony X Single-coupling procedure”, “Symphony X Single-Coupling Single-shot Procedure”; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-65% B over 15 minutes, then a 2-minute hold at 100% B; Flow: 45 mL/min. Fractions containing the desired product were combined and dried via centrifugal evaporation. The yield of the product was 5.4 mg, and its estimated purity by LCMS analysis was 97%.
Analysis condition A: Retention time=1.55 min; ESI-MS(+) m/z [M+2H]²⁺: 925.0.
Analysis condition B: Retention time=1.72 min: ESI-MS(+) m/z [M+2H]²⁺: 925.2.

### Preparation of Example 1091

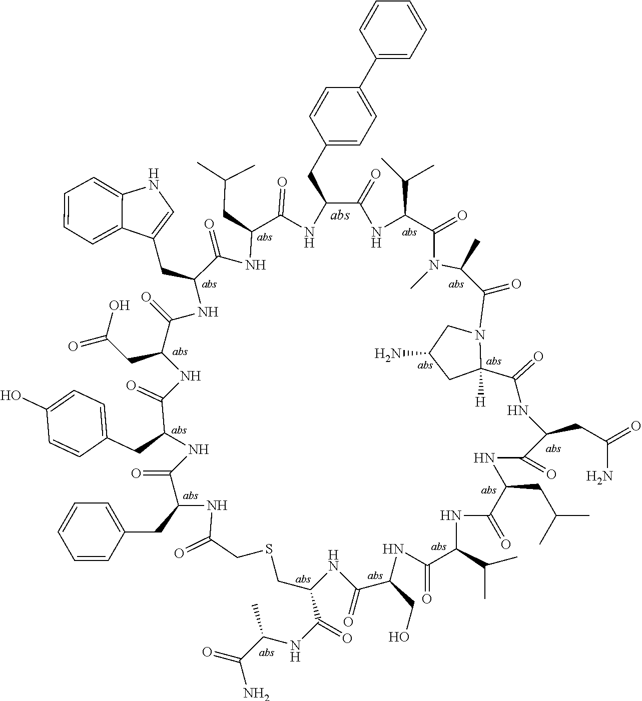 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)[C@H]2C[C@H](N)CN2C(=O)[C@H](C)N(C)C(=O)[C@H](C(C)C)NC(=O)[C@H](Cc2ccc(-c3ccccc3)cc2)NC1=O]

Example 1091 was prepared, using Sieber or Rink on a −1 μmol scale, following the general synthetic sequence described for the preparation of Example 1000 composed of the following general procedures: “Symphony Resin-swelling procedure”, “Symphony Single-coupling procedure”, Single-Coupling Pre-Activation Procedure” for Fmoc-D-Pro(4-NHBoc)-OH, “Symphony Chloroacetic Anhydride coupling procedure”, “Global Deprotection Method A”, and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-55% B over 25 minutes, then a 5-minute hold at 100% B; Flow: 20 mL/min. Fractions containing the desired product were combined and dried via centrifugal evaporation. The yield of the product was 18.5 mg, and its estimated purity by LCMS analysis was 91.7%.

Analysis condition B: Retention time=1.55 min; ESI-MS(+) m/z [M+2H]²⁺: 945.6.

### Preparation of Example 1092

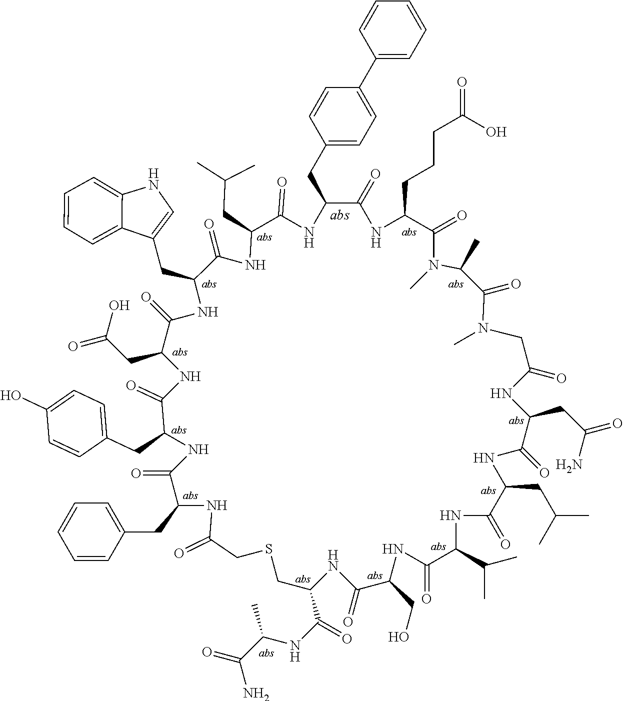 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](CCCC(=O)O)NC(=O)[C@H](Cc2ccc(-c3ccccc3)cc2)NC1=O]

Example 1092 was prepared, using Sieber or Rink on a −1 μmol scale, following the general synthetic sequence described for the preparation of Example 1000 composed of the following general procedures: “Symphony Resin-swelling procedure”, “Symphony Single-coupling procedure”, Single-Coupling Pre-Activation Procedure “Symphony Chloroacetic Anhydride coupling procedure”, “Global Deprotection Method A”, and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-55% B over 25 minutes, then a 5-minute hold at 100% Flow: 20 mL/min. Fractions containing the desired product were combined and dried via centrifugal evaporation. The yield of the product was 27.3 mg, and its estimated purity by LCMS analysis was 95.9%.
Analysis condition A: Retention time=1.63 min; ESI-MS(+) m/z [M+H]⁺: 1893.2.
Analysis condition B: Retention time=1.82 min; ESI-MS(+) m/z [M+H]⁺: 1893.2.

### Preparation of Example 1093

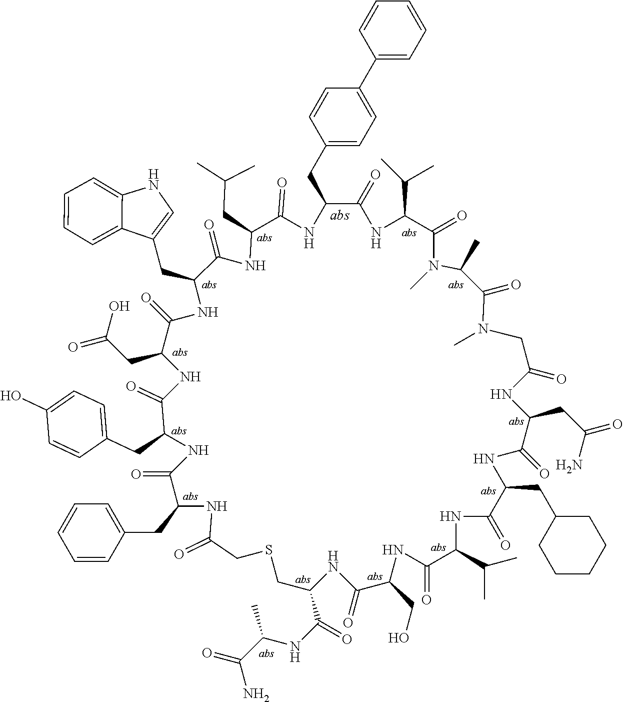 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC2CCCCC2)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1093 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, Single-Coupling Pre-Activation Procedure” for Fmoc-Cha-OH, “Symphony Chloroacetic Anhydride coupling procedure”, “Global Deprotection Method A”, and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-70% B over 20 minutes, then a 5-minute hold at 100% B; Flow: 20 mL/min. Fractions containing the desired product were combined and dried via centrifugal evaporation. The yield of the product was 12 mg, and its estimated purity by LCMS analysis was 94.7%.
Analysis condition A: Retention time=1.72 min; ESI-MS(+) m/z [M+H]⁺: 1888.0.
Analysis condition B: Retention time=1.93 min; ESI-MS(+) m/z [M+2H]²⁺: 944.9.

### Preparation of Example 1094

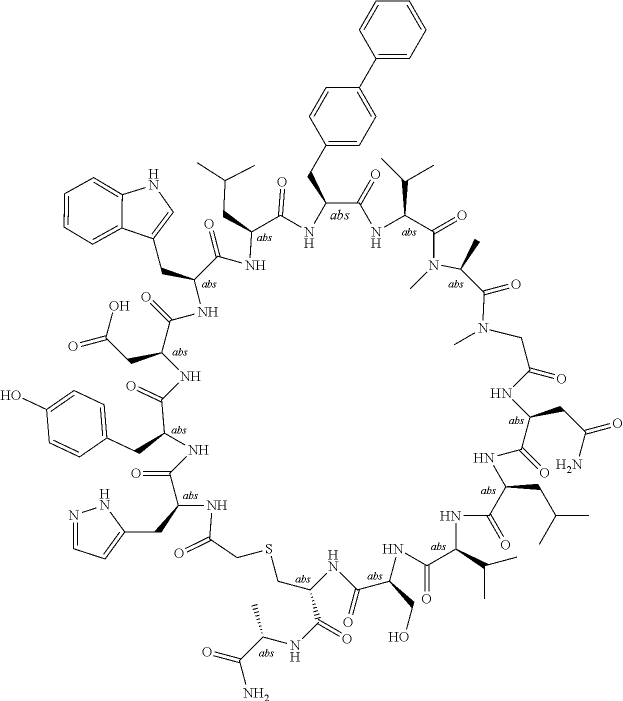 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccn[nH]2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1094 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Single-Coupling Manual Addition Procedure B” was followed; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×150 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 10-60% B over 20 minutes, then a 2-minute hold at 100% B; Flow: 40 mL/min. Fractions containing the desired product were combined and dried via centrifugal evaporation. The yield of the product was 14.3 mg, and its estimated purity by LCMS analysis was 84.5%.
Analysis condition A: Retention time=1.52 min; ESI-MS(+) m/z [M+H]⁺: 1837.8.
Analysis condition B: Retention time=1.69 min; ESI-MS(+)m m/z [M+H]⁺: 1837.8.

### Preparation of Example 1095

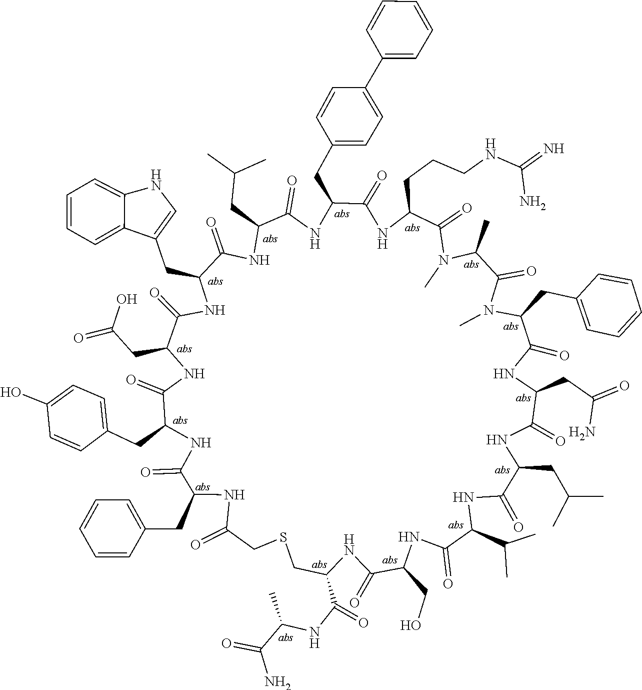 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC1=O]

Example 1095 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 30-70% B over 20 minutes, then a 5-minute hold at 100% B; Flow: 20 mL/min. Fractions containing the desired product were combined and dried via centrifugal evaporation. The yield of the product was 38.6 mg, and its estimated purity by LCMS analysis was 93.5%.
Analysis condition A: Retention time=2.21 min; ESI-MS(+) m/z [M+H]⁺: 1995.1.
Analysis condition B: Retention time=1.96 min; ESI-MS(+) m/z [M+H]⁺: 1994.9.

### Preparation of Example 1096

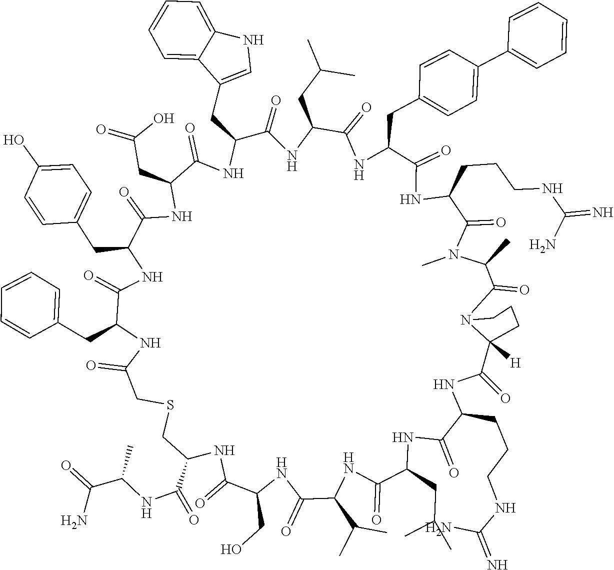

Example 1096 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 18-58% B over 27 minutes, then a 3-minute hold at 100% Flow: 20 mL/min. Fractions containing the desired product were combined and dried via centrifugal evaporation. The yield of the product was 27.5 mg, and its estimated purity by LCMS analysis was 92.8%.
Analysis condition A: Retention time=2.3 min; ESI-MS(+) m/z [M+H]⁺: 1974.3.
Analysis condition B: Retention time=1.59 min; ESI-MS(+) m/z [M+H]⁺: 1973.1.

### Preparation of Example 1097

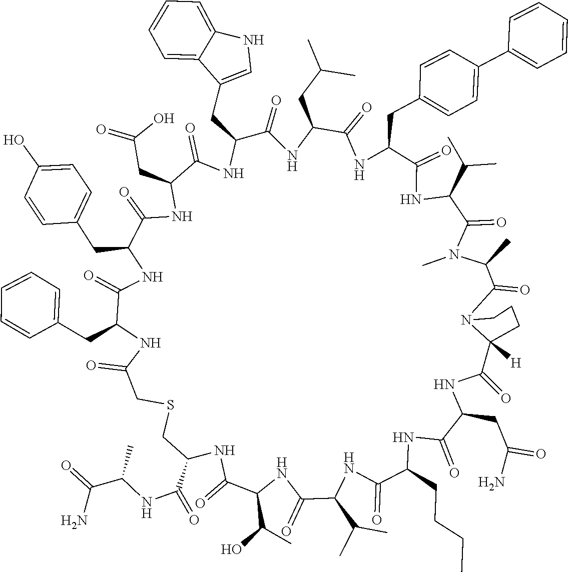

Example 1097 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, “Symphony double-coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 25 minutes, then a 5-minute hold at 100% B; Flow: 20 mL/min. Fractions containing the desired product were combined and dried via centrifugal evaporation. The yield of the product was 30.7 mg, and its estimated purity by LCMS analysis was 98%.
Analysis condition A: Retention time=1.64 min; ESI-MS(+) m/z [M+H]⁺: 1888.1.
Analysis condition B: Retention time=1.83 min; ESI-MS(+) m/z [M+H]⁺: 1888.1.

### Preparation of Example 1098

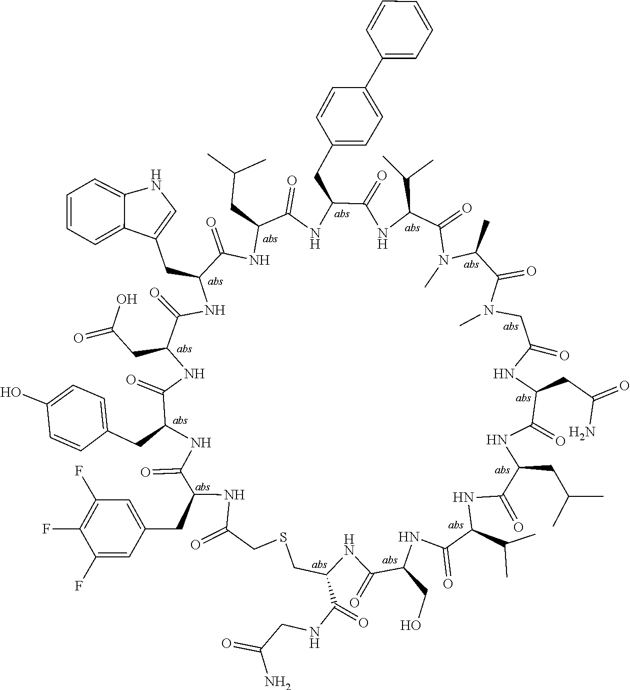 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2cc(F)c(F)c(F)c2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1098 was prepared, using Sieber or ink on a 50 μmol scale, following the general synthetic sequence described for the preparation of Example 1054 composed of the following general procedures: “Prelude Resin-swelling procedure”, “Prelude Single-coupling procedure”, “Symphony X Resin-swelling procedure”, “Symphony X Single-coupling procedure”, “Symphony X Single-Coupling Single-shot Procedure” was followed with Fmoc-Phe(3,4,5-triF)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×150 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 20-70% B over 20 minutes, then a 2-minute hold at 100% B; Flow: 40 mL/min. Fractions containing the desired product were combined and dried via centrifugal evaporation. The yield of the product was 25.2 mg, and its estimated purity by LCMS analysis was 100%.
Analysis condition A: Retention time=1.65 min; ESI-MS(+) m/z [M+H]⁺: 1888.9.
Analysis condition B: Retention time=1.88 min; ESI-MS(+) m/z [M+H]⁺: 1887.9.

### Preparation of Example 1099

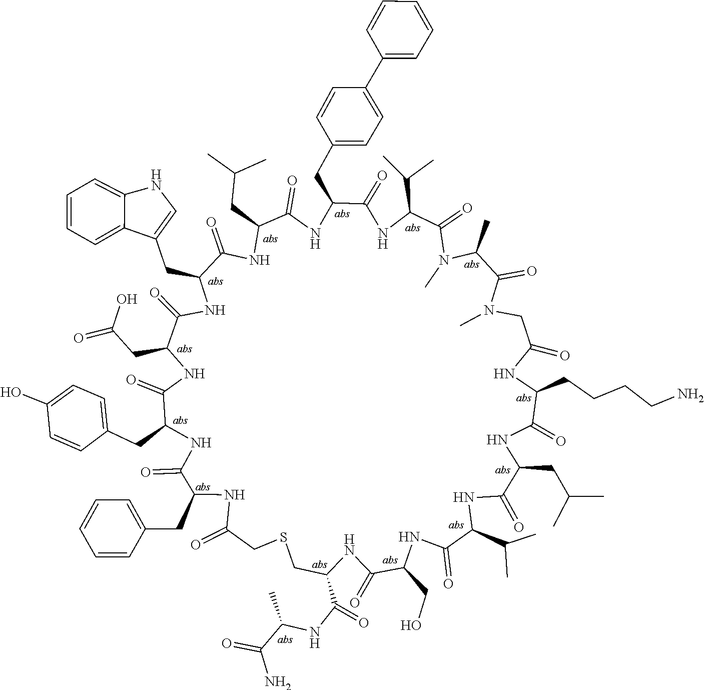 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CCCCN)NC(=O)CN(C)C(=O)[C@H](C)N(C)C(=O)[C@H](C(C)C)NC(=O)[C@H](Cc2ccc(-c3ccccc3)cc2)NC1=O]

Example 1099 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×150 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 15-65% B over 20 minutes, then a 2-minute hold at 100% B; Flow: 40 mL/min. Fractions containing the desired product were combined and dried via centrifugal evaporation. The yield of the product was 15.4 mg, and its estimated purity by LCMS analysis was 88.6%.
Analysis condition A: Retention time=1.65 min; ESI-MS(+) m/z [M+H]⁺: 1861.9.
Analysis condition B: Retention time=1.69 min; ESI-MS(+) m/z [M+H]⁺: 1862.0.

### Preparation of Example 1100

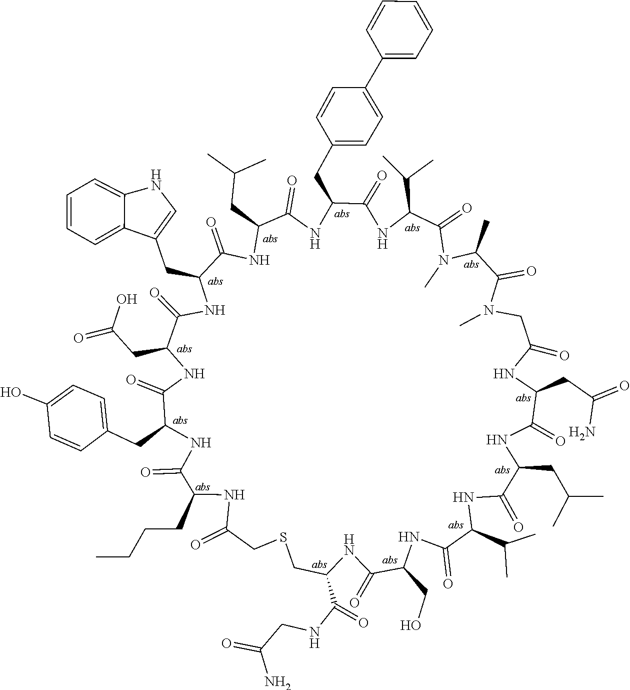 [SMILES: CCCC[C@@H]1NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(O)cc2)NC1=O]

Example 1100 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 20 minutes, then a 5-minute hold at 100% B; Flow: 20 mL/min. Fractions containing the desired product were combined and dried via centrifugal evaporation. The yield of the product was 27.9 mg, and its estimated purity by LCMS analysis was 100%.

Analysis condition A Retention time=1.54 min; ESI-MS(+) m/z [M+2H]²⁺: 900.9.
Analysis condition B: Retention time=1.72 min; ESI-MS(+) m/z [M+2H]²⁺: 901.1. Preparation of Example I101

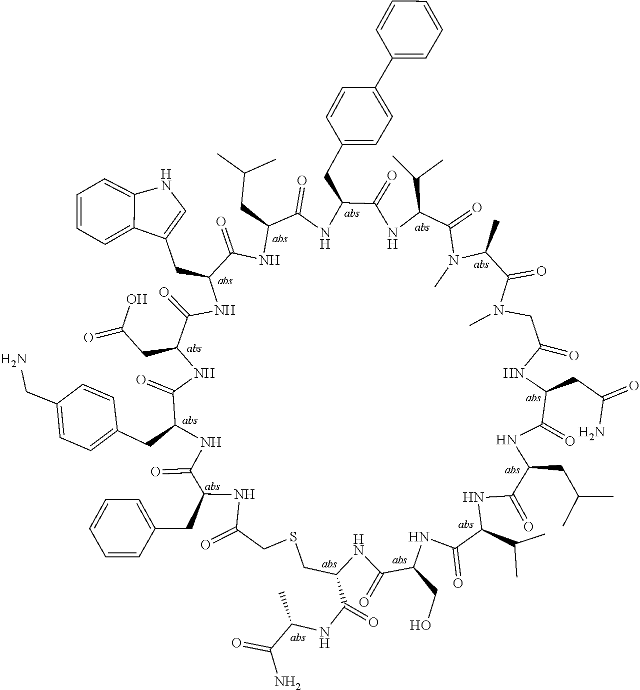 [SMILES: CC(C)C[C@@H]1NC(=O)[C@H](Cc2c[nH]c3ccccc23)NC(=O)[C@H](CC(=O)O)NC(=O)[C@H](Cc2ccc(CN)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1101 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Single-Coupling Manual Addition Procedure B” was followed with Fmoc-D-Hyp-OH; “Symphony X Chloroacetic Anhydride coupling procedure”. “Symphony X Final rinse and dry procedure”, “Global Deprotection Method A”, “Cyclization Method A”.

The crude material was purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-65% B over 20 minutes, then a 2-minute hold at 100% B; Flow: 45 mL/min. Fractions containing the desired product were combined and dried via centrifugal evaporation. The yield of the product was 22.9 mg, and its estimated purity by LCMS analysis was 97.3%.
Analysis condition A: Retention time=1.55 min; ESI-MS(+) m/z [M+2H]²⁺: 931.4.
Analysis condition B: Retention time=1.65 min; ESI-MS(+) m/z [M+2H]²⁺: 931.2.

### Preparation of Example 1102

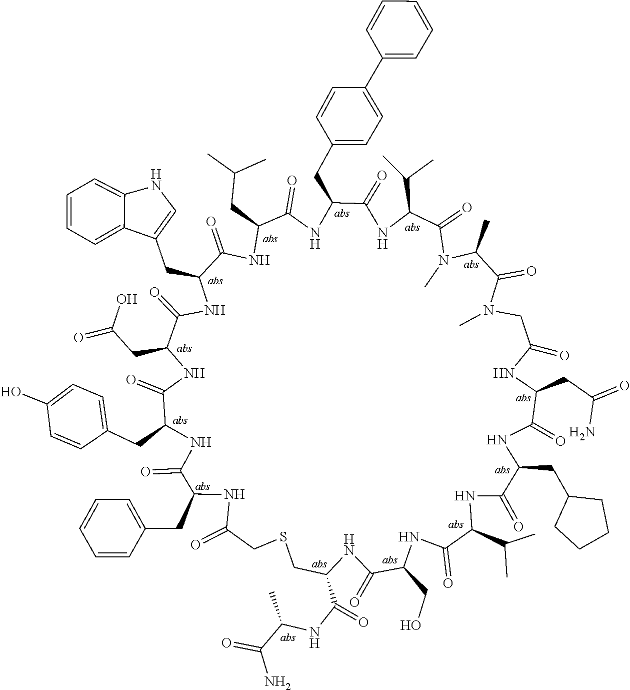 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC2CCCC2)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1102 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, Single-Coupling Pre-Activation Procedure” for Fmoc-cyclopenty-Ala-OH, “Symphony Chloroacetic Anhydride coupling procedure”, “Global Deprotection Method A”, and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 19 minutes, then a 5-minute hold at 100% B; Flow: 20 mL/min. Fractions containing the desired product were combined and dried via centrifugal evaporation. The yield of the product was 11.9 mg, and its estimated purity by LCMS analysis was 100%.
Analysis condition A: Retention time=1.6 min; ESI-MS(+) m/z [M+2H]²⁺: 938.2.
Analysis condition B: Retention time=1.76 min; ESI-MS(+) m/z [M+2H]²⁺: 938.0.

### Preparation of Example 1103

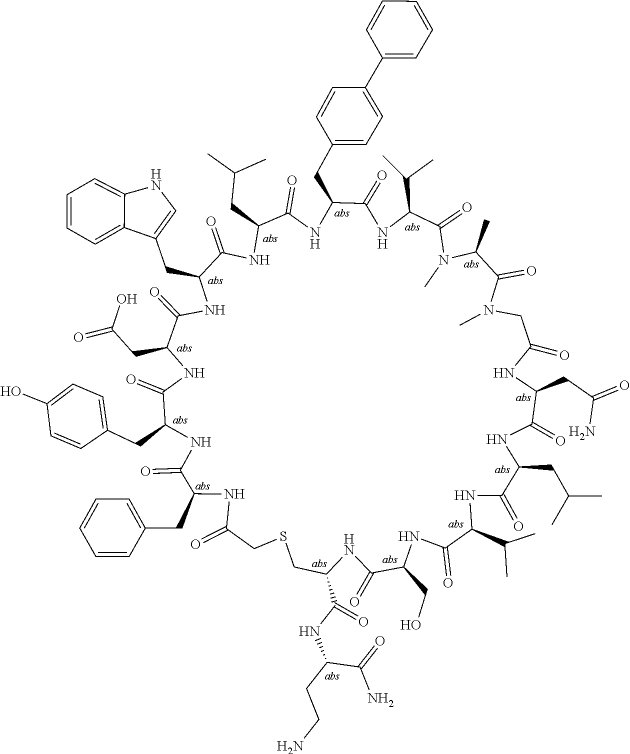 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](CCN)C(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1103 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, Single-Coupling Pre-Activation Procedure” for Fmoc-Dab(Boc)-OH, “Symphony Chloroacetic Anhydride coupling procedure”, “Global Deprotection Method A”, and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 20 minutes, then a 5-minute hold at 100% B; Flow: 20 mL/min. Fractions containing the desired product were combined and dried via centrifugal evaporation. The material was further purified via preparative LC/MS with the following conditions: Column: XBridge C18, 30×150 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 15-65% B over 20 minutes, then a 2-minute hold at 100% B; Flow: 40 mL/min. Fractions containing the desired product were combined and dried via centrifugal evaporation. The yield of the product was 10.7 mg, and its estimated purity by LCMS analysis was 96.1%.
Analysis condition A: Retention time=1.6 min; ESI-MS(+) m/z [M+H]⁺: 1878.1.
Analysis condition B: Retention time=1.72 min; ESI-MS(+) m/z [M+H]⁺: 1876.9.

### Preparation of Example 1104

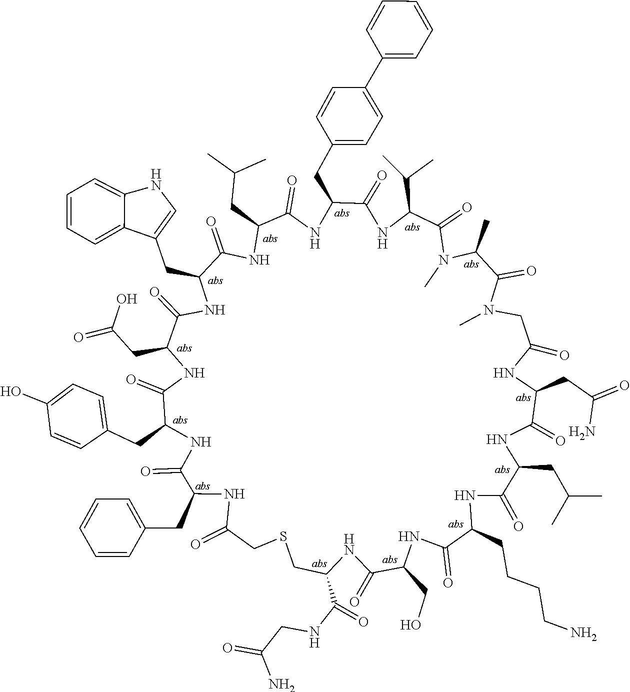 [SMILES: CC(C)C[C@@H]1NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](CCCCN)NC1=O]

Example 1104 was prepared, using Sieber or Rink on a 50 μmol scale, following the general synthetic sequence described for the preparation Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method B”, “Cyclization Method A”.

The crude material was purified via preparative LC/MS with the following conditions: Column: XBridge C18, 19×200 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 20-60% B over 20 minutes, then a 5-minute hold at 100% B; Flow: 20 mL/min. Fractions containing the desired product were combined and dried via centrifugal evaporation. The yield of the product was 22.4 mg, and its estimated purity by LCMS analysis was 84.6%.
Analysis condition A: Retention time=1.71 min; ESI-MS(+) m/z [M+H]⁺: 1862.9.
Analysis condition B: Retention time=1.53 min; ESI-MS(+) m/z [M+H]⁺: 1862.7.

### Preparation of Example 1105

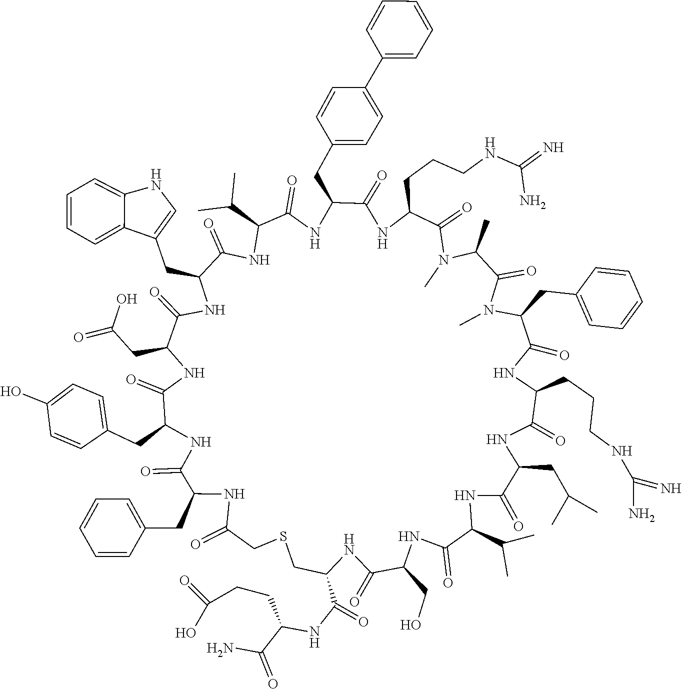 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](CCC(=O)O)C(N)=O)NC(=O)[C@H](CO)NC(=O)[C@H](C(C)C)NC1=O]

Example 1105 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×150 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 20-70% B over 20 minutes, then a 2-minute hold at 100% B; Flow: 40 mL/min. Fractions containing the desired product were combined and dried via centrifugal evaporation. The material was further purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-65% B over 20 minutes, then a 2-minute hold at 100% B; Flow: 45 mL/min. Fractions containing the desired product were combined and dried via centrifugal evaporation. The yield of the product was 1.1 mg, and its estimated purity by LCMS analysis was 91.2%.
Analysis condition A: Retention time=1.77 min; ESI-MS(+) m/z [M+2H]²⁺: 1041.3.
Analysis condition B: Retention time=1.8 min; ESI-MS(+) m/z [M+2H]²⁺: 1041.0.

### Preparation of Example 1106

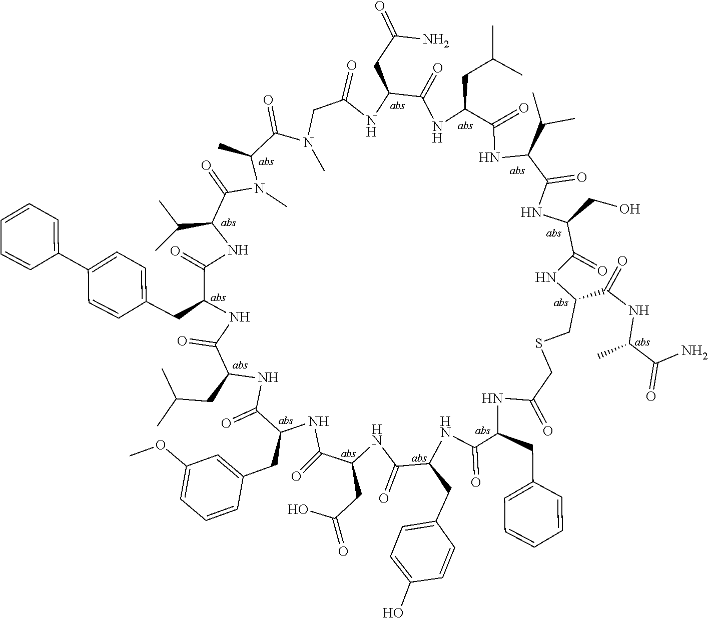 [SMILES: COc1cccc(C[C@@H]2NC(=O)[C@H](CC(=O)O)NC(=O)[C@H](Cc3ccc(O)cc3)NC(=O)[C@H](Cc3ccccc3)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 1106 was prepared, using Sieber or Rink on a 4620 μmol scale, following the general synthetic sequence described for the preparation of Example 1054, composed of the following general procedures: “Prelude Resin-swelling procedure”, “Prelude Single-coupling procedure”, “Symphony Resin-swelling procedure”, “Symphony Single-coupling procedure”, “Symphony Single-Coupling Pre-activation Procedure” was followed with Fmoc-Phe(3-OMe)-OH; “Symphony Chloroacetic Anhydride coupling procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-55% B over 20 minutes, then a 5-minute hold at 100% B; Flow: 20 mL/min. Fractions containing the desired product were combined and dried via centrifugal evaporation. The yield of the product was 900 mg, and its estimated purity by LCMS analysis was 97.7%.
Analysis condition A: Retention time=1.79 min; ESI-MS(+) m/z [M+NH₄]⁺: 1855.5.
Analysis condition B: Retention time=1.89 min; ESI-MS(+) m/z [M+H]⁺: 1840.1.

### Preparation of Example 1107

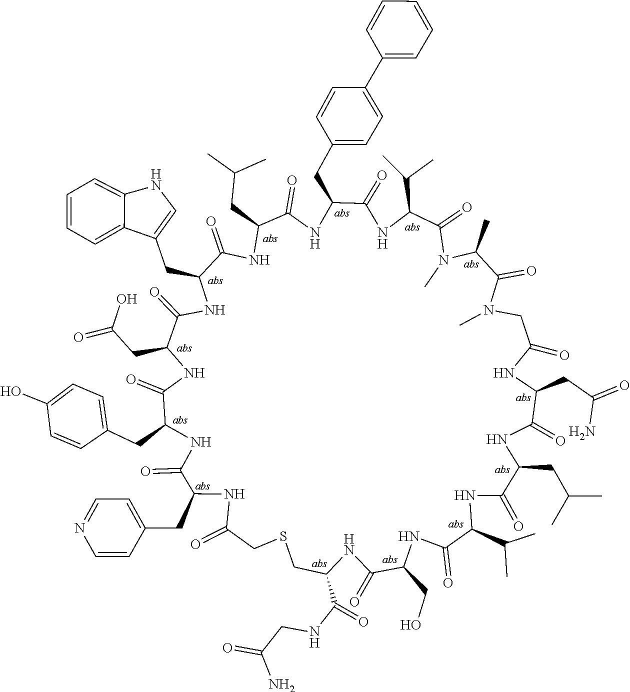 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccncc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1107 was prepared, using Sieber or Rink on a 50 μmol scale, following the general synthetic sequence described for the preparation of Example 1054 composed of the following general procedures: “Prelude Resin-swelling procedure”, “Prelude Single-coupling procedure”, “Symphony X Resin-swelling procedure”, “Symphony X Single-coupling procedure”, “Symphony X Single-Coupling Single-shot Procedure” was followed with Fmoc-Ala(4-Pyr)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×150 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 15-65% B over 20 minutes, then a 2-minute hold at 100% B; Flow: 40 mL/min. Fractions containing the desired product were combined and dried via centrifugal evaporation. The yield of the product was 29.1 mg, and its estimated purity by LCMS analysis was 98.2%.
Analysis condition A: Retention time=1.48 min; ESI-MS(+) m/z [M+H]⁺: 1835.0.
Analysis condition B: Retention time=1.64 min; ESI-MS(+) m/z [M+H]⁺: 1836.1

### Preparation of Example 1108

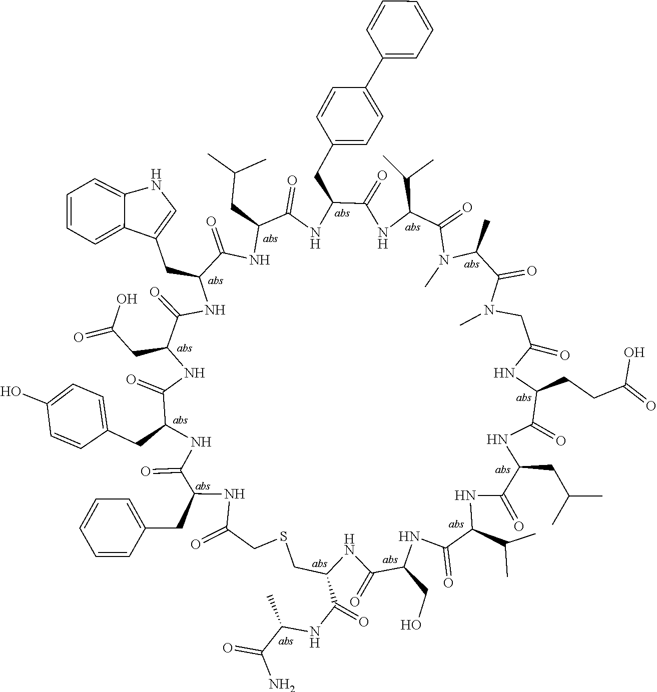 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CCC(=O)O)NC(=O)CN(C)C(=O)[C@H](C)N(C)C(=O)[C@H](C(C)C)NC(=O)[C@H](Cc2ccc(-c3ccccc3)cc2)NC1=O]

Example 1108 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×200 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 15-65% B over 20 minutes, then a 2-minute hold at 100% B; Flow: 40 mL/min. Fractions containing the desired product were combined and dried via centrifugal evaporation. The material was further purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 5-55% B over 20 minutes, then a 2-minute hold at 100% B; Flow: 45 mL/min. Fractions containing the desired product were combined and dried via centrifugal evaporation. The yield of the product was 1.6 mg, and its estimated purity by LCMS analysis was 100%. Analysis condition A: Retention time=1.45 min; ESI-MS(+) m/z [M+2H]²⁺: 932.1.
Analysis condition B: Retention time=1.75 min; ESI-MS(+) m/z [M+2H]²⁺: 932.0.

### Preparation of Example 1109

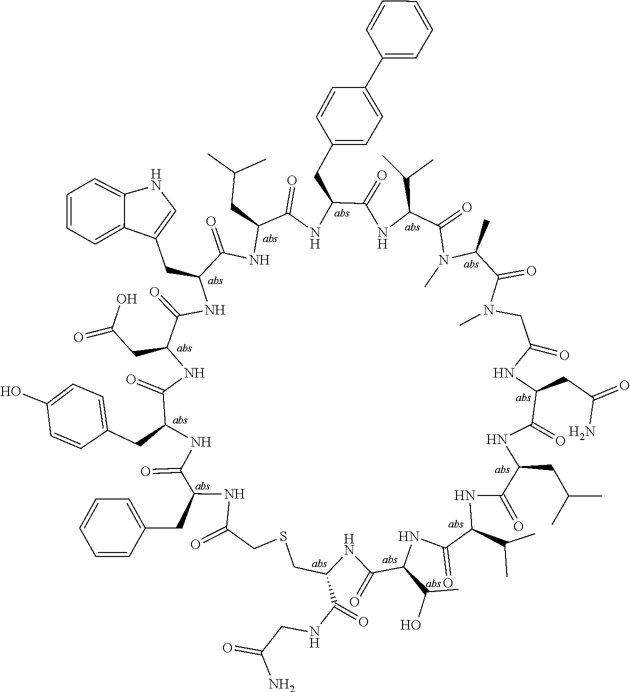 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](C(C)O)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1109 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×150 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 15-65% B over 20 minutes, then a 2-minute hold at 100% B; Flow: 40 mL/min. Fractions containing the desired product were combined and dried via centrifugal evaporation. The yield of the product was 40 mg, and its estimated purity by LCMS analysis was 100%.
Analysis condition A: Retention time=1.68 min; ESI-MS(+) m/z [M+H]⁺: 1848.0.
Analysis condition B: Retention time=1.78 min; ESI-MS(+) m/z [M+H]⁺: 1848.0

### Preparation of Example 1110

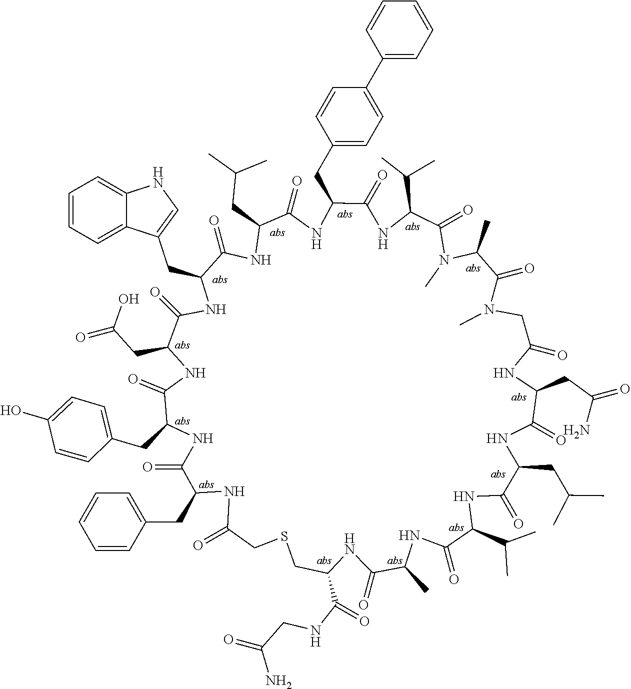 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](C)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1110 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”,

“Symphony X Chloroacetic Anhydride coupling procedure”, “Symphony X Final rinse and dry procedure”, “Global Deprotection Method B”, “Cyclization Method A”.

The crude material was purified via preparative LC/MS with the following conditions: Column: XBridge C18, 19×200 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 30-70% B over 20 minutes, then a 5-minute hold at 100% B; Flow: 20 mL/min. Fractions containing the desired product were combined and dried via centrifugal evaporation. The yield of the product was 20.5 mg, and its estimated purity by LCMS analysis was 99%.
Analysis condition A: Retention time=1.57 min; ESI-MS(+) m/z [M+2H]²⁺: 910.1.
Analysis condition B: Retention time=1.72 min; ESI-MS(+) m/z [M+2H]²⁺: 910.1.

### Preparation of Example 1111

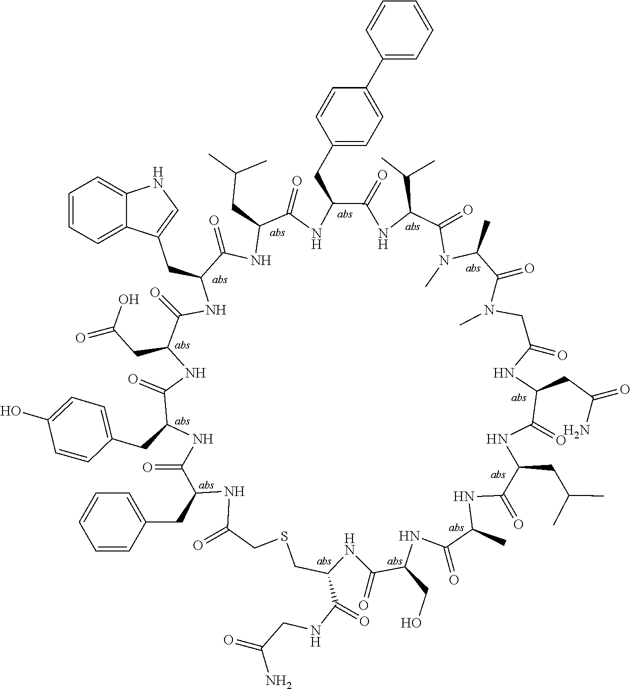 [SMILES: CC(C)C[C@@H]1NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C)NC1=O]

Example 1111 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”,

“Symphony X Chloroacetic Anhydride coupling procedure”, “Symphony X Final rinse and dry procedure”, “Global Deprotection Method B”, “Cyclization Method A”.

The crude material was purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-65% B over 20 minutes, then a 2-minute hold at 100% B; Flow: 45 mL/min. Fractions containing the desired product were combined and dried via centrifugal evaporation. The yield of the product was 20.4 mg, and its estimated purity by LCMS analysis was 87.1%.
Analysis condition A: Retention time=1.61 min; ESI-MS(+) m/z [M+H]⁺: 1805.9.
Analysis condition B: Retention time=1.61 min; ESI-MS(+) m/z [M+H]⁺: 1805.6.

### Preparation of Example 1112

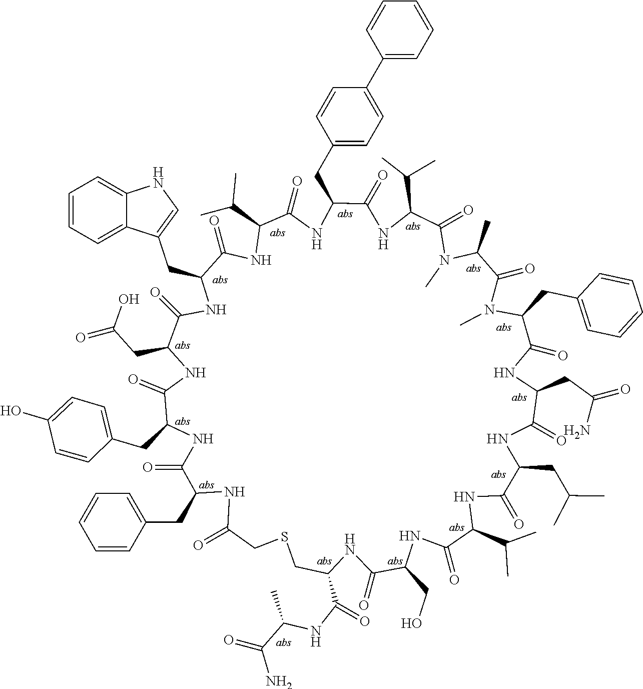 [SMILES: CC(C)C[C@@H]1NC(=O)[C@H](CC(N)=O)NC(=O)[C@H](Cc2ccccc2)N(C)C(=O)[C@H](C)N(C)C(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC1=O]

Example 1112 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 10-70% B over 20 minutes, then a 4-minute hold at 100% B; Flow: 20 mL/min. Fractions containing the desired product were combined and dried via centrifugal evaporation. The yield of the product was 47.2 mg, and its estimated purity by LCMS analysis was 100%.
Analysis condition A: Retention time=2.06 min; ESI-MS(+) m/z [M+H]⁺: 1925.1.
Analysis condition B: Retention time=2.19 min; ESI-MS(+) m/z [M+H]⁺: 1924.3.

### Preparation of Example 1113

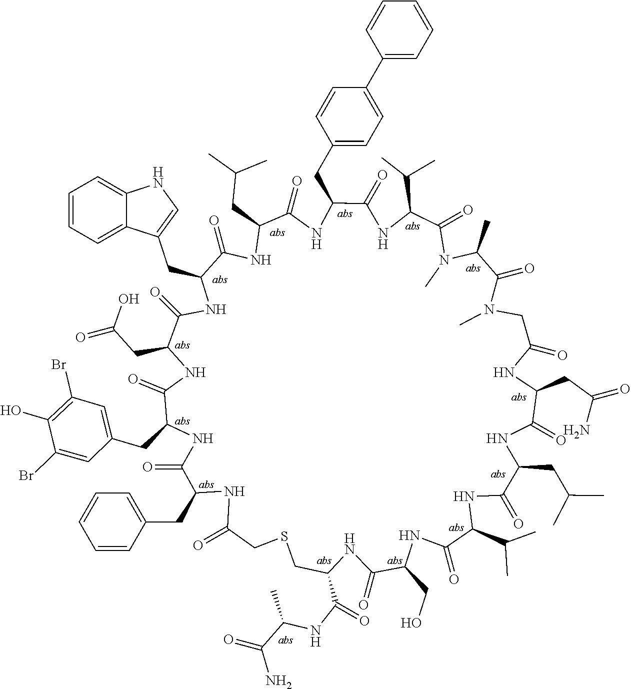 [SMILES: CC(C)C[C@@H]1NC(=O)[C@H](Cc2c[nH]c3ccccc23)NC(=O)[C@H](CC(=O)O)NC(=O)[C@H](Cc2cc(Br)c(O)c(Br)c2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1113 was prepared, using Sieber or Rink on a 50 μmol scale, following the general synthetic sequence described for the preparation of Example 1054, composed of the following general procedures: “Prelude Resin-swelling procedure”, “Prelude Single-coupling procedure”, “Symphony X Resin-swelling procedure”, “Symphony X Single-coupling procedure”, “Symphony X Single-Coupling Manual Addition Procedure B” was followed with Fmoc-Tyr(3,5-diBr)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-70% B over 20 minutes, then a 2-minute hold at 100% B; Flow: 45 mL/min. Fractions containing the desired product were combined and dried via centrifugal evaporation. The yield of the product was 10.9 mg, and its estimated purity by LCMS analysis was 97.5%.
Analysis condition A: Retention time=1.76 min; ESI-MS(+) m/z [M+2H]²⁺: 1004.1.
Analysis condition B: Retention time=1.94 min; ESI-MS(+) m/z [M+2H]²⁺: 1003.2.

### Preparation of Example 1114

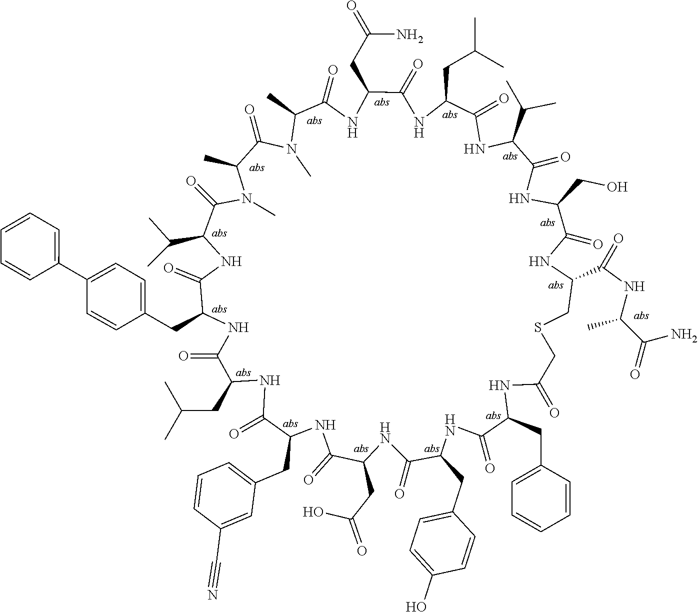 [SMILES: CC(C)C[C@@H]1NC(=O)[C@H](Cc2cccc(C#N)c2)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)[C@H](C)N(C)C(=O)[C@H](C)N(C)C(=O)[C@H](C(C)C)NC(=O)[C@H](Cc2ccc(-c3ccccc3)cc2)NC1=O]

Example 1114 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, Single-Coupling Pre-Activation Procedure” for Fmoc-Phe(3-CN)—OH, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 20 minutes, then a 4-minute hold at 100% B; Flow: 20 mL/min. Fractions containing the desired product were combined and dried via centrifugal evaporation. The yield of the product was 33 mg, and its estimated purity by LCMS analysis was 98.3%.
Analysis condition A: Retention time=1.48 min; ESI-MS(+) m/z [M+2H]²⁺: 918.0.
Analysis condition B: Retention time=1.66 min; ESI-MS(+) m/z [M+2H]²⁺: 918.1.

### Preparation of Example 1115

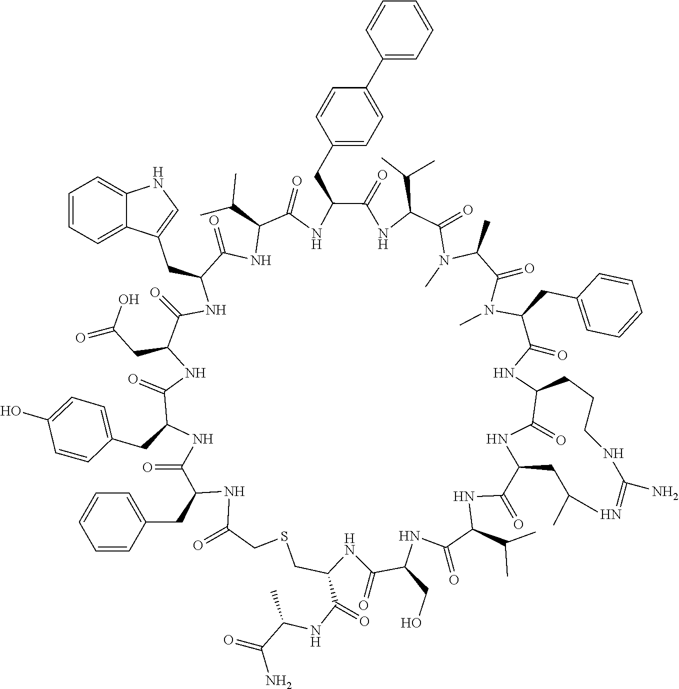 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC1=O]

Example 1115 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 30-70% B over 20 minutes, then a 5-minute hold at 100% B; Flow: 20 mL/min. Fractions containing the desired product were combined and dried via centrifugal evaporation. The yield of the product was 10.4 mg, and its estimated purity by LCMS analysis was 99.3%.
Analysis condition A: Retention time=2.06 min; ESI-MS(+) m/z [M+H]⁺: 1966.0.
Analysis condition B: Retention time=1.95 min; ESI-MS(+) m/z [M+2H]²⁺: 983.9.

### Preparation of Example 1116

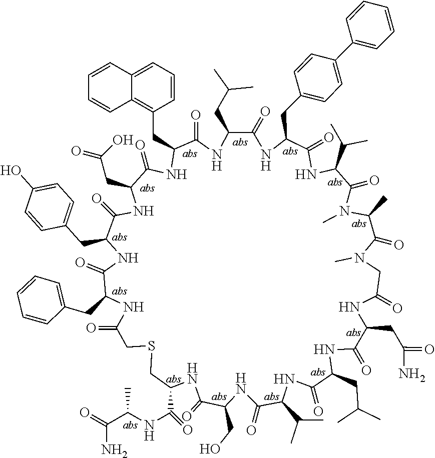

Example 1116 was prepared, using Sieber or Rink on a 50 μmol scale, following the general synthetic sequence described for the preparation of Example 1054, composed of the following general procedures: “Prelude Resin-swelling procedure”, “Prelude Single-coupling procedure”, “Symphony X Resin-swelling procedure”, “Symphony X Single-coupling procedure”, “Symphony X Single-Coupling Manual Addition Procedure B” was followed with Fmoc-Ala(1-Naphthyl)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-70% B over 20 minutes, then a 2-minute hold at 100% B; Flow: 45 mL/min. Fractions containing the desired product were combined and dried via centrifugal evaporation. The yield of the product was 34.8 mg, and its estimated purity by LCMS analysis was 98.2%.
Analysis condition A: Retention time=1.71 min; ESI-MS(+) m/z [M+H]⁺: 1859.9.
Analysis condition B: Retention time=1.93 min; ESI-MS(+) m/z [M+H]⁺: 1860.0.

### Preparation of Example 1117

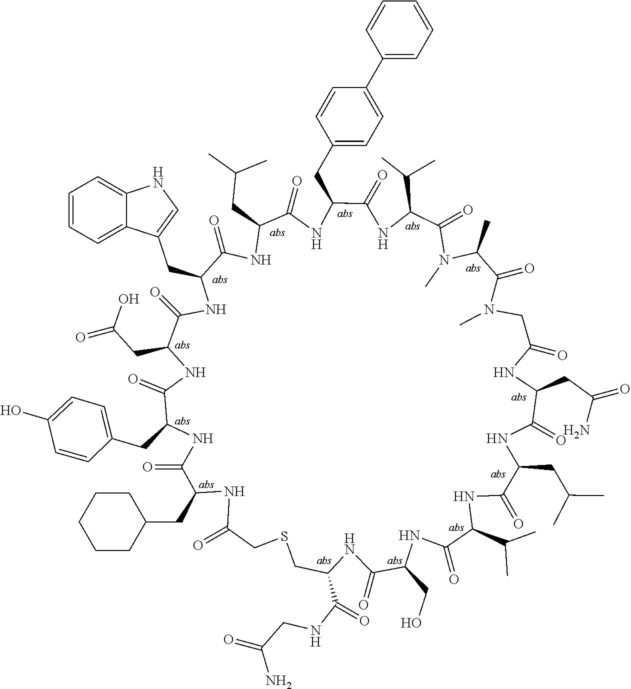 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](CC2CCCCC2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1117 was prepared, using Sieber or Rink on a 50 μmol scale, following the general synthetic sequence described for the preparation of Example 1054, composed of the following general procedures: “Prelude Resin-swelling procedure”, “Prelude Single-coupling procedure”, “Symphony Resin-swelling procedure”, “Symphony Single-coupling procedure”, “Symphony Single-Coupling Pre-activation Procedure” was followed with Fmoc-Cha-OH; “Symphony Chloroacetic Anhydride coupling procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 20 minutes, then a 5-minute hold at 100% B; Flow: 20 mL/min. Fractions containing the desired product were combined and dried via centrifugal evaporation. The yield of the product was 19 mg, and its estimated purity by LCMS analysis was 100%.
Analysis condition A: Retention time=1.65 min; ESI-MS(+) m/z [M+2H]²⁺: 921.2.
Analysis condition B: Retention time=1.82 min; ESI-MS(+) m/z [M+2H]²⁺: 921.2.

### Preparation of Example 1118

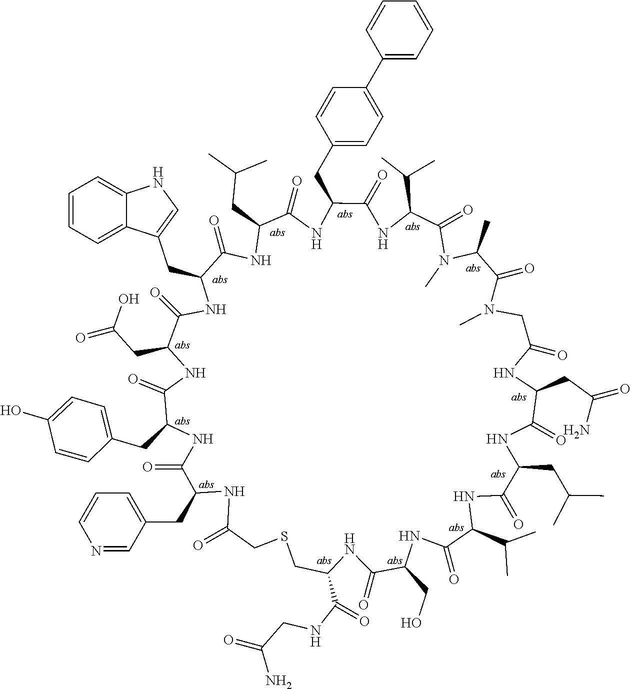 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2cccnc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1118 was prepared, using Sieber or Rink on a 50 μmol scale, following the general synthetic sequence described for the preparation of Example 1054 composed of the following general procedures: “Prelude Resin-swelling procedure”, “Prelude Single-coupling procedure”, “Symphony X Resin-swelling procedure”, “Symphony X Single-coupling procedure”, “Symphony X Single-Coupling Single-shot Procedure” was followed with Fmoc-Ala(3-Pyr)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 10-60% B over 20 minutes, then a 2-minute hold at 100% B; Flow: 45 mL/min. Fractions containing the desired product were combined and dried via centrifugal evaporation. The yield of the product was 27.9 mg, and its estimated purity by LCMS analysis was 99.1%.
Analysis condition A: Retention time=1.49 min; ESI-MS(+) m/z [M+H]⁺: 1835.0.
Analysis condition B: Retention time=1.62 min; ESI-MS(+) m/z [M+H]⁺: 1835.2.

### Preparation of Example 1119

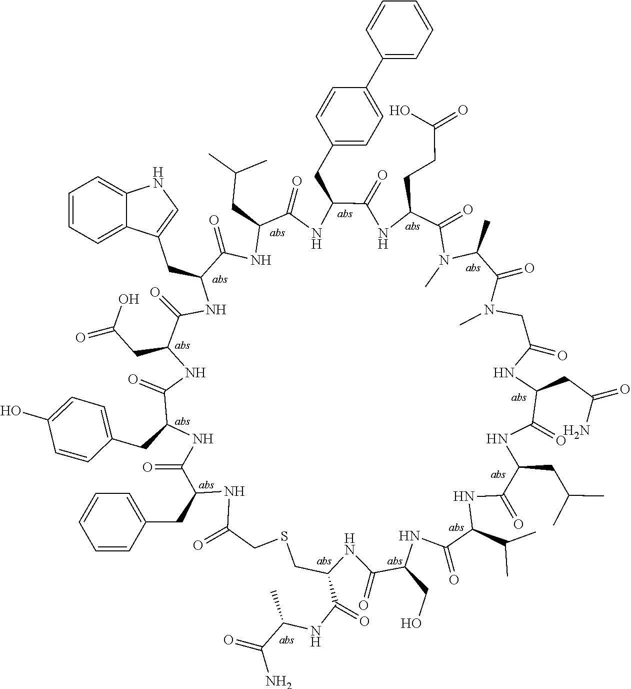 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](CCC(=O)O)NC(=O)[C@H](Cc2ccc(-c3ccccc3)cc2)NC1=O]

Example 1119 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 20 minutes, then a 5-minute hold at 100% B; Flow: 20 mL/min. Fractions containing the desired product were combined and dried via centrifugal evaporation. The yield of the product was 7.1 mg, and its estimated purity by LCMS analysis was 98.1%.
Analysis condition A: Retention time=1.51 min; ESI-MS(+) m/z [M+H]⁺: 1877.8.
Analysis condition B: Retention time=1.78 min; ESI-MS(+) m/z [M+H]⁺: 1877.9.

### Preparation of Example 1120

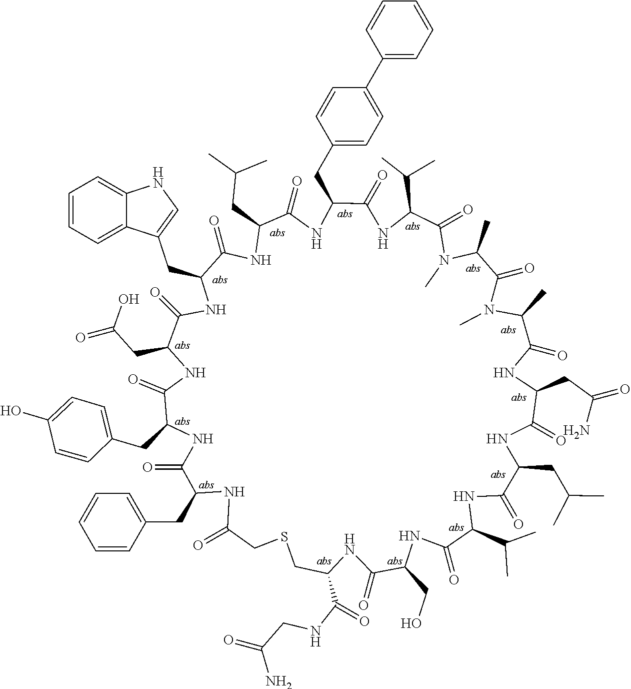 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CC(N)=O)NC(=O)[C@H](C)N(C)C(=O)[C@H](C)N(C)C(=O)[C@H](C(C)C)NC(=O)[C@H](Cc2ccc(-c3ccccc3)cc2)NC1=O]

Example 1120 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, “Symphony double-coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 30-70% B over 20 minutes, then a 5-minute hold at 100% B; Flow: 20 mL/min. Fractions containing the desired product were combined and dried via centrifugal evaporation. The material was further purified via preparative LC/MS with the following conditions: Column: XBridge C18, 30×150 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 20-70% B over 20 minutes, then a 2-minute hold at 100% B; Flow: 40 mL/min. Fractions containing the desired product were combined and dried via centrifugal evaporation. The yield of the product was 28.2 mg, and its estimated purity by LCMS analysis was 97.4%.
Analysis condition A: Retention time=1.65 min; ESI-MS(+) m/z [M+2H]²⁺: 925.3.
Analysis condition B: Retention time=1.81 min; ESI-MS(+) m/z [M+2H]²⁺: 924.9.

### Preparation of Example 1121

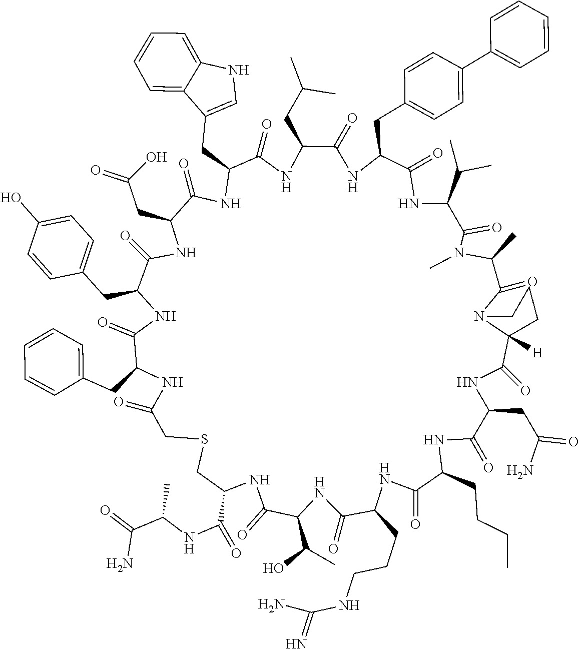

Example 1121 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Single-shot Procedure” was followed for Fmoc-Ne—OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 20-60% B over 25 minutes, then a 5-minute hold at 100% B; Flow: 20 mL/min. Fractions containing the desired product were combined and dried via centrifugal evaporation. The yield of the product was 17.3 mg, and its estimated purity by LCMS analysis was 98%.
Analysis condition 1: Retention time=1.68, 1.71 min; ESI-MS(+)m z [M+2H]²⁺: 973.25, 973.62.
Analysis condition 1: Retention time=1.8 min; ESI-MS(+) m/z [M+3H]³⁺: 649.2.

### Preparation of Example 1122

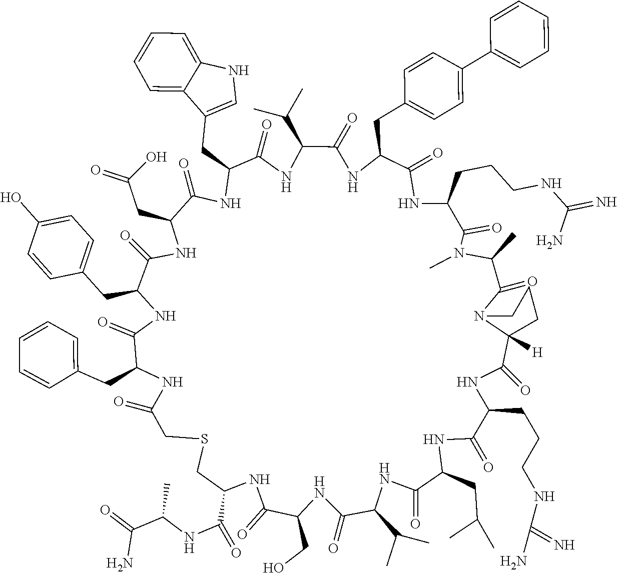

Example 1122 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-55% B over 20 minutes, then a 5-minute hold at 100% B; Flow: 20 mL/min. Fractions containing the desired product were combined and dried via centrifugal evaporation. The yield of the product was 24.5 mg, and its estimated purity by LCMS analysis was 96.8%.
Analysis condition A: Retention time=1.76 min; ESI-MS(+) m/z [M+H]⁺: 1960.1.
Analysis condition B: Retention time=1.73 min; ESI-MS(+) m/z [M+H]⁺: 1959.3.

### Preparation of Example 1123

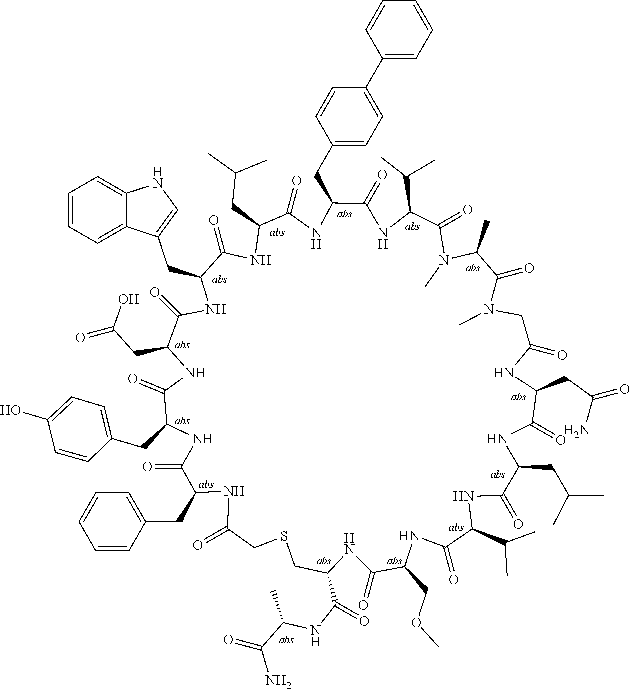 [SMILES: COC[C@@H]1NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC1=O]

Example 1123 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, Single-Coupling Pre-Activation Procedure” for Fmoc-Ser(Me)-OH, “Symphony Chloroacetic Anhydride coupling procedure”, “Global Deprotection Method A”, and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 20 minutes, then a 5-minute hold at 100% B; Flow: 20 mL/min. Fractions containing the desired product were combined and dried via centrifugal evaporation. The yield of the product was 20 mg, and its estimated purity by LCMS analysis was 97.4%.
Analysis condition A: Retention time=1.61 min; ESI-MS(+) m/z [M+2H]²⁺: 932.0.
Analysis condition B: Retention time=1.72 min; ESI-MS(+) m/z [M+2H]²⁺: 932.3.

### Preparation of Example 1124

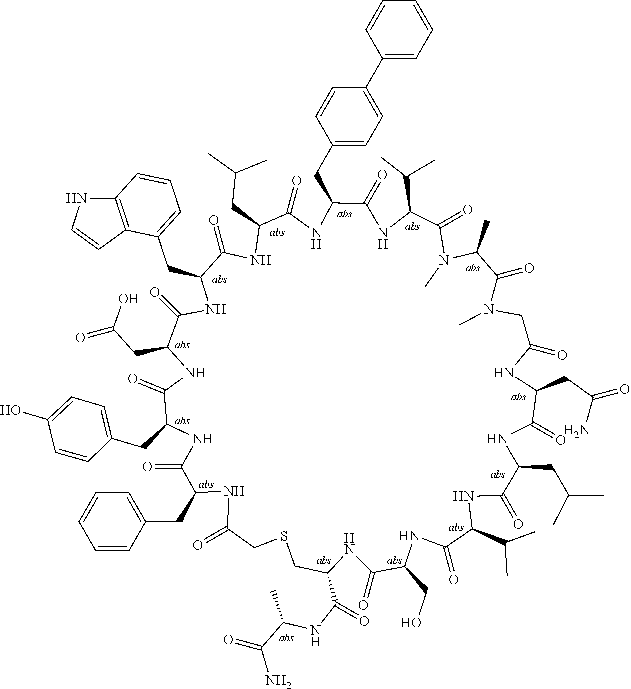 [SMILES: CC(C)C[C@@H]1NC(=O)[C@H](Cc2cccc3[nH]ccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1124 was prepared, using Sieber or Rink on a 50 μmol scale, following the general synthetic sequence described for the preparation of Example 1054, composed of the following general procedures: “Prelude Resin-swelling procedure”, “Prelude Single-coupling procedure”, “Symphony X Resin-swelling procedure”, “Symphony X Single-coupling procedure”, “Symphony X Single-Coupling Manual Addition Procedure B” was followed with Fmoc-Iso-Trp(Boc)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-65% B over 20 minutes, then a 2-minute hold at 100% B; Flow: 45 mL/min. Fractions containing the desired product were combined and dried via centrifugal evaporation. The yield of the product was 10.8 mg, and its estimated purity by LCMS analysis was 92.3%.
Analysis condition A: Retention time=1.7 min; ESI-MS(+) m/z [M+H]⁺: 1848.0.
Analysis condition B: Retention time=1.75 min; ESI-MS(+) m/z [M+H]⁺: 1848.1.

### Preparation of Example 1125

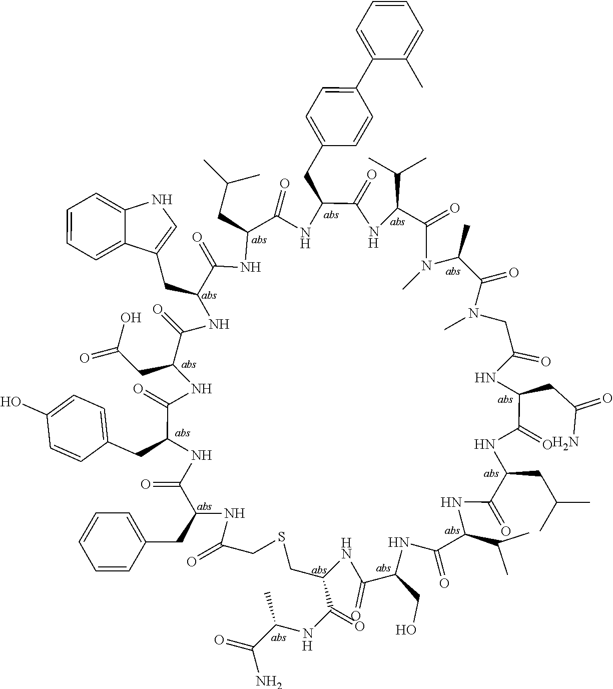

Example 1125 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, Single-Coupling Pre-Activation Procedure” for Fmoc-Bip(2′-Me)-OH, “Symphony Chloroacetic Anhydride coupling procedure”, “Global Deprotection Method A”, and “Cyclization Method”.

The crude material was purified via preparative LC/MS with the following conditions: Column: XBridge C18, 19×200 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 30-70% B over 19 minutes, then a 5-minute hold at 100% B; Flow: 20 mL/min. Fractions containing the desired product were combined and dried via centrifugal evaporation. The material was further purified via preparative LC/MS with the following conditions: Column: XBridge C18, 30×150 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 20-75% B over 20 minutes, then a 2-minute hold at 100% B; Flow: 40 mL/min. Fractions containing the desired product were combined and dried via centrifugal evaporation. The yield of the product was 8.3 mg, and its estimated purity by LCMS analysis was 96.5%.
Analysis condition A: Retention time=1.64 min; ESI-MS(+) m/z [M+2H]²⁺: 932.0.
Analysis condition B: Retention time=1.82 min; ESI-MS(+) m/z [M+2H]²⁺: 932.2.

### Preparation of Example 1126

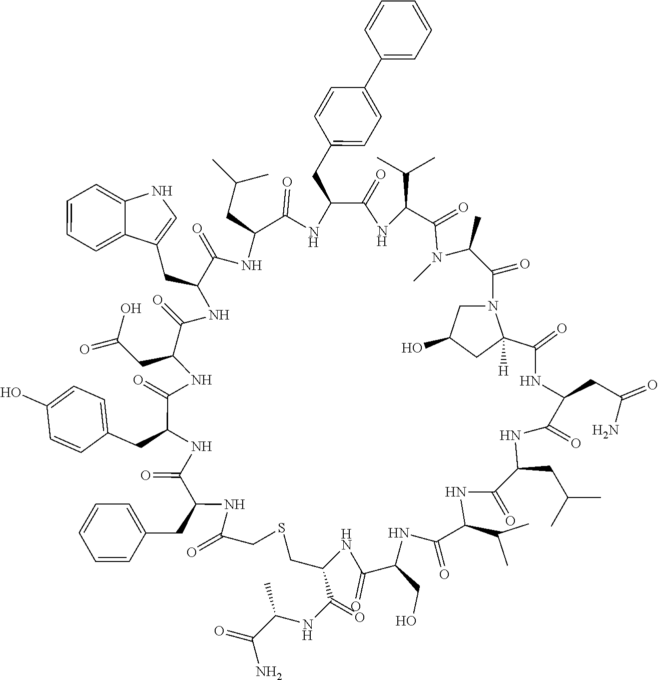

Example 1126 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Single-Coupling Manual Addition Procedure B” was followed with Fmoc-D-Hyp-OH; “Symphony X Chloroacetic Anhydride coupling procedure”. “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 20-60% B over 20 minutes, then a 5-minute hold at 100% B; Flow: 20 mL/min. Fractions containing the desired product were combined and dried via centrifugal evaporation. The yield of the product was 8.1 mg, and its estimated purity by LCMS analysis was 84.1%.
Analysis condition A: Retention time=1.75 min; ESI-MS(+) m/z [M+H]⁺: 1890.1.
Analysis condition B: Retention time=1.86 min; ESI-MS(+) m/z [M+H]⁺: 1890.1.

### Preparation of Example 1127

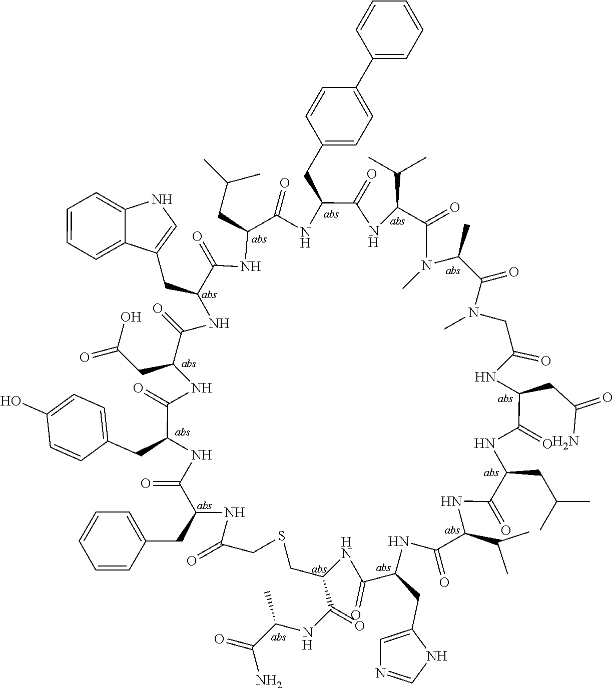

Example 1127 was prepared, using Sieber or Rink on a 50 μmol scale, following the general synthetic sequence described for the preparation of Example 1054, composed of the following general procedures: “Prelude Resin-swelling procedure”, “Prelude Single-coupling procedure”, “Symphony Resin-swelling procedure”, “Symphony Single-coupling procedure”, “Symphony Chloroacetic Anhydride coupling procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 20-60% B over 20 minutes, then a 4-minute hold at 100% B; Flow: 20 mL/min. Fractions containing the desired product were combined and dried via centrifugal evaporation. The yield of the product was 15.5 mg, and its estimated purity by LCMS analysis was 100%.
Analysis condition A: Retention time=1.54 min; ESI-MS(+) m/z [M+2H]²⁺: 950.1.
Analysis condition B: Retention time=1.65 min; ESI-MS(+) m/z [M+2H]²⁺: 950.2.

### Preparation of Example 1128

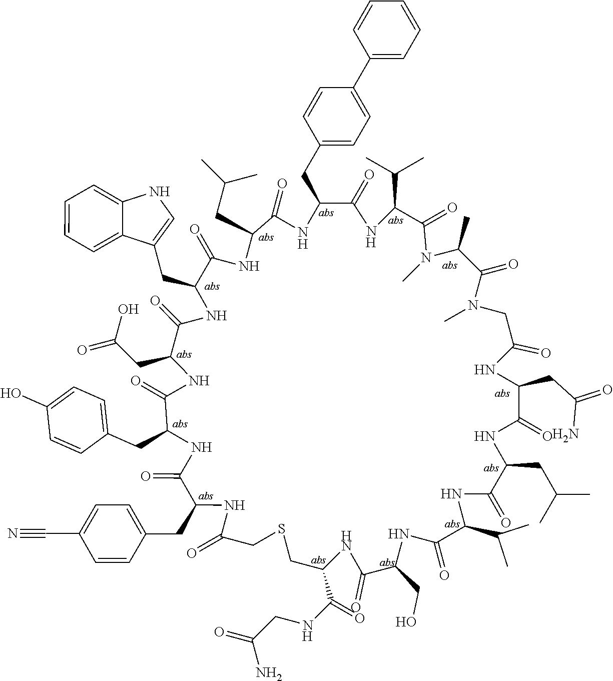

Example 1128 was prepared, using Sieber or Rink on a 50 μmol scale, following the general synthetic sequence described for the preparation of Example 1054 composed of the following general procedures: “Prelude Resin-swelling procedure”, “Prelude Single-coupling procedure”, “Symphony X Resin-swelling procedure”, “Symphony X Single-coupling procedure”, “Symphony X Single-Coupling Single-shot Procedure” was followed with Fmoc-Phe(4-CN)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×150 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 15-65% B over 20 minutes, then a 2-minute hold at 100% B; Flow: 40 mL/min. Fractions containing the desired product were combined and dried via centrifugal evaporation. The yield of the product was 30.8 mg, and its estimated purity by LCMS analysis was 100%.
Analysis condition A: Retention time=1.56 min; ESI-MS(+) m/z [M+H]⁺: 1860.0.
Analysis condition B: Retention time=1.79 min; ESI-MS(+) m/z [M+H]⁺: 1860.2.

### Preparation of Example 1129

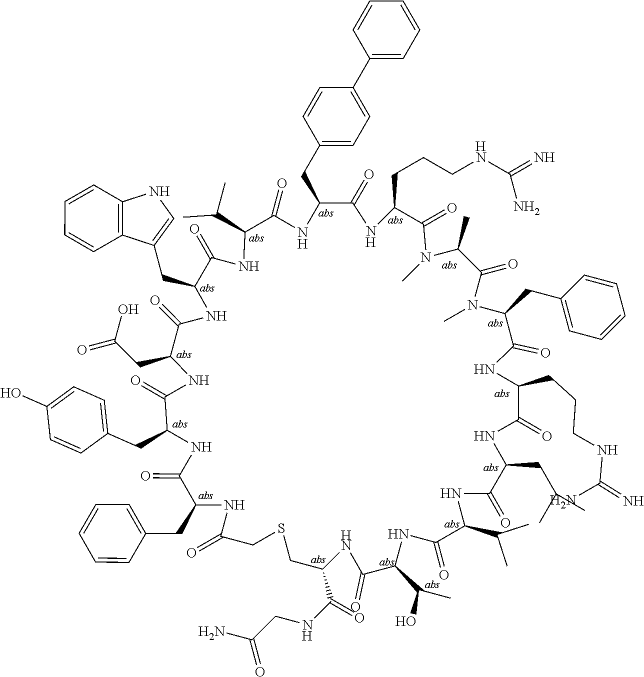

Example 1129 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, “Symphony Chloroacetic Anhydride coupling procedure”, “Global Deprotection Method A”, and “Cyclization Method”.

The crude material was purified via preparative LC/MS with the following conditions: Column: XBridge C18, 30×200 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 15-65% B over 20 minutes, then a 2-minute hold at 100% B; Flow: 45 mL/min. Fractions containing the desired product were combined and dried via centrifugal evaporation. The material was further purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-70% B over 20 minutes, then a 2-minute hold at 100% B; Flow: 45 mL/min. Fractions containing the desired product were combined and dried via centrifugal evaporation. The yield of the product was 11.5 mg, and its estimated purity by LCMS analysis was 95.4%.
Analysis condition A: Retention time=1.75 min; ESI-MS(+) m/z [M+2H]²⁺: 1012.9.
Analysis condition B: Retention time=1.79 min; ESI-MS(+) m/z [M+3H]³⁺: 675.3.

### Preparation of Example 1130

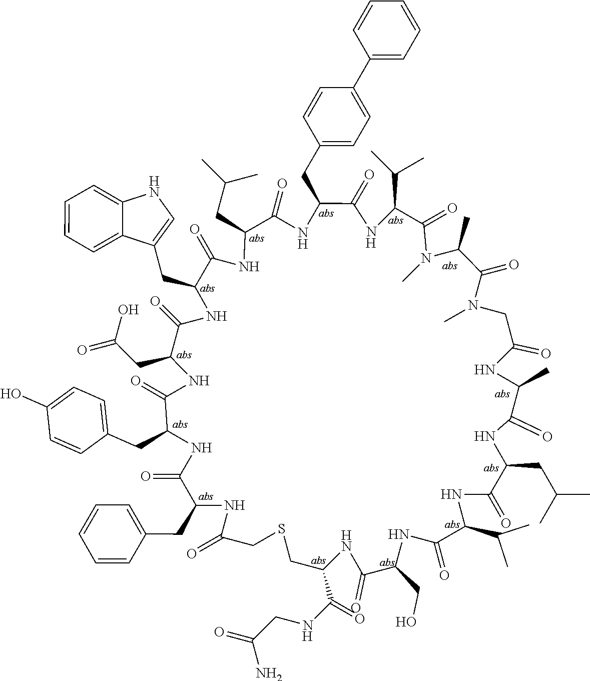

Example 1130 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”,

“Symphony X Chloroacetic Anhydride coupling procedure”, “Symphony X Final rinse and dry procedure”, “Global Deprotection Method B”, “Cyclization Method A”.

The crude material was purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 10-60% B over 20 minutes, then a 2-minute hold at 100% B; Flow: 45 mL/min. Fractions containing the desired product were combined and dried via centrifugal evaporation. The yield of the product was 18 mg, and its estimated purity by LCMS analysis was 100%.
Analysis condition A: Retention time=1.76 min; ESI-MS(+) m/z [M+H]⁺: 1792.1.
Analysis condition B: Retention time=1.79 min; ESI-MS(+) m/z [M−H]⁻: 1789.0.

### Preparation of Example 1131

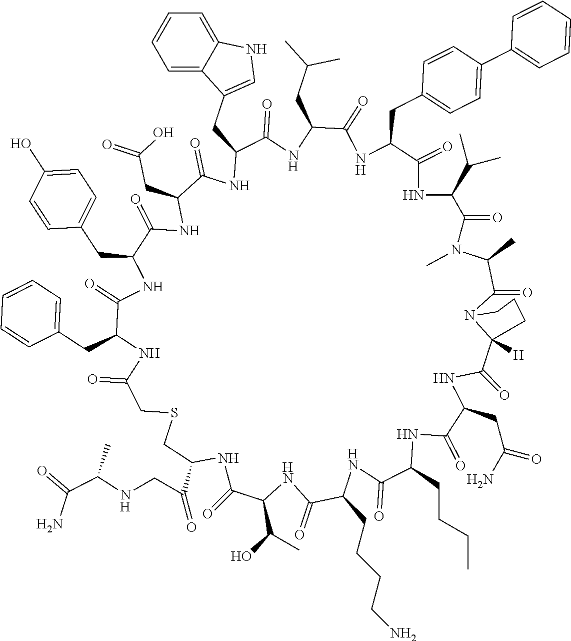

Example 1131 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Single-shot Procedure” was followed for Fmoc-Nle-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 23-63% B over 25 minutes, then a 5-minute hold at 100% B; Flow: 20 mL/min. Fractions containing the desired product were combined and dried via centrifugal evaporation. The yield of the product was 17.3 mg, and its estimated purity by LCMS analysis was 100%.
Analysis condition A: Retention time=1.69 min; ESI-MS(+) m/z [M+2H]²⁺: 959.2.
Analysis condition B: Retention time=1.79 min; ESI-MS(+) m/z [M+3H]³⁺: 640.0.

### Preparation of Example 1132

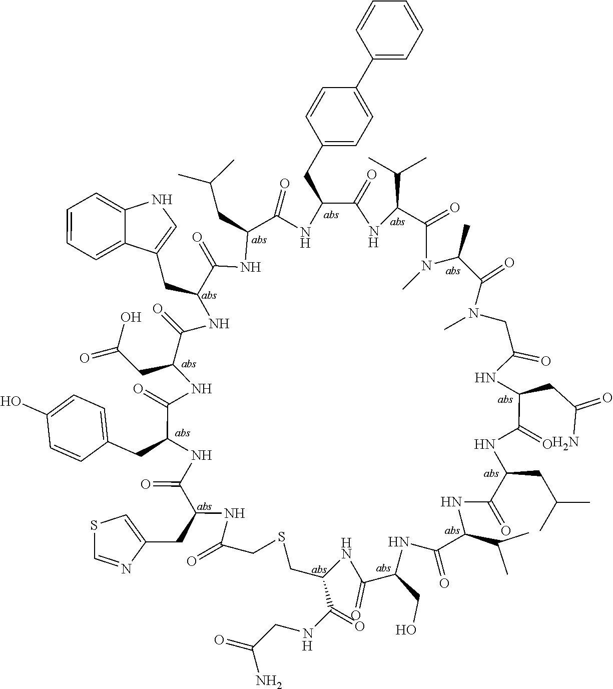

Example 1132 was prepared, using Sieber or Rink on a 50 μmol scale, following the general synthetic sequence described for the preparation of Example 1054 composed of the following general procedures: “Prelude Resin-swelling procedure”, “Prelude Single-coupling procedure”, “Symphony X Resin-swelling procedure”, “Symphony X Single-coupling procedure”, “Symphony X Single-Coupling Single-shot Procedure” was followed; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×150 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 15-65% B over 20 minutes, then a 2-minute hold at 100% B; Flow: 40 mL/min. Fractions containing the desired product were combined and dried via centrifugal evaporation. The yield of the product was 28.2 mg, and its estimated purity by LCMS analysis was 99%.
Analysis condition A: Retention time=1.5 min; ESI-MS(+) m/z [M+H]⁺: 1841.2.
Analysis condition B: Retention time=1.72 min; ESI-MS(+) m/z [M+Na]⁺: 1864.1.

### Preparation of Example 1133

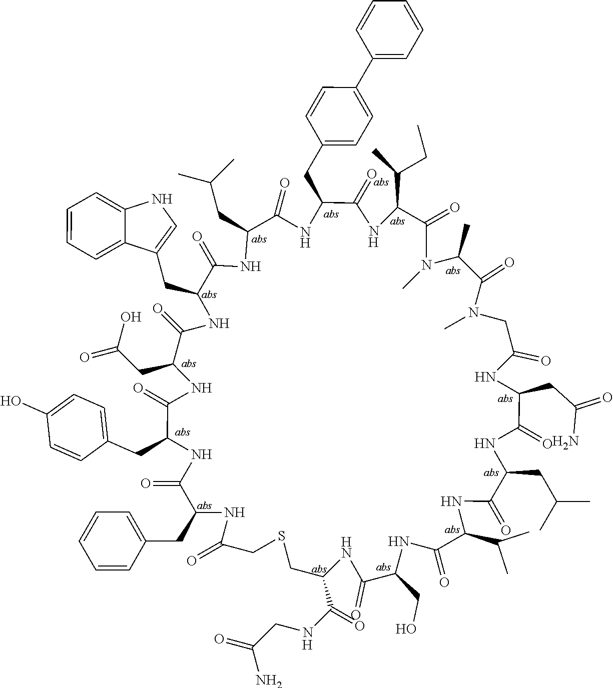

Example 1133 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×150 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 20-70% B over 20 minutes, then a 2-minute hold at 100% B; Flow: 40 mL/min. Fractions containing the desired product were combined and dried via centrifugal evaporation. The yield of the product was 21.9 mg, and its estimated purity by LCMS analysis was 98.6%.
Analysis condition A: Retention time=1.57 min; ESI-MS(+) m/z [M+2H]²⁺: 924.7.
Analysis condition B: Retention time=1.7, 1.73 min; ESI-MS(+) m/z [M+2H]²⁺: 924.8, 924.8.

### Preparation of Example 1134

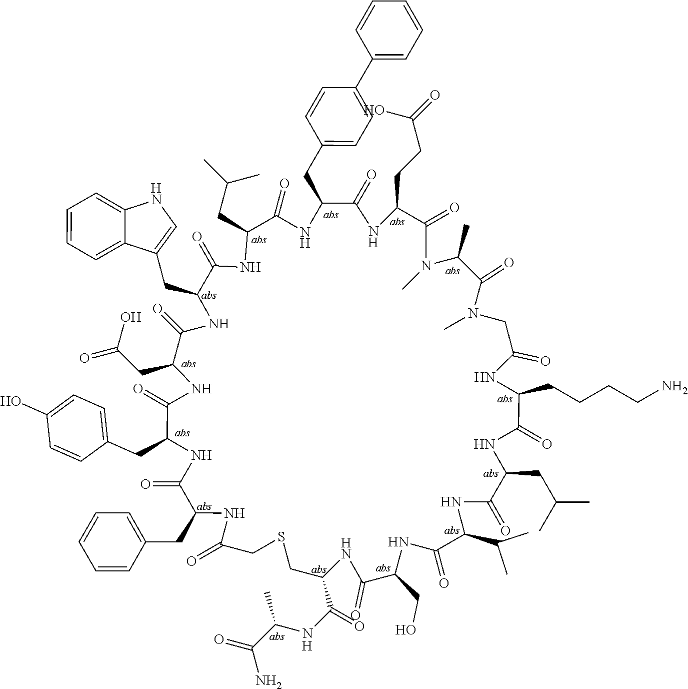

Example 1134 was prepared, using Sieber or Rink on a 50 μmol scale, following the general synthetic sequence described for the preparation of Example 1000 composed of the following general procedures: “Symphony Resin-s welling procedure”, “Symphony Single-coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-55% B over 20 minutes, then a 5-minute hold at 100% B; Flow: 20 mL/min. Fractions containing the desired product were combined and dried via centrifugal evaporation. The yield of the product was 10.2 mg, and its estimated purity by LCMS analysis was 98.6%.
Analysis condition A: Retention time=1.47 min; ESI-MS(+) m/z [M+2H]²⁺: 946.4.
Analysis condition B: Retention time=1.56 min: ESI-MS(+) m/z [M+2H]²⁺: 946.2.

### Preparation of Example 1135

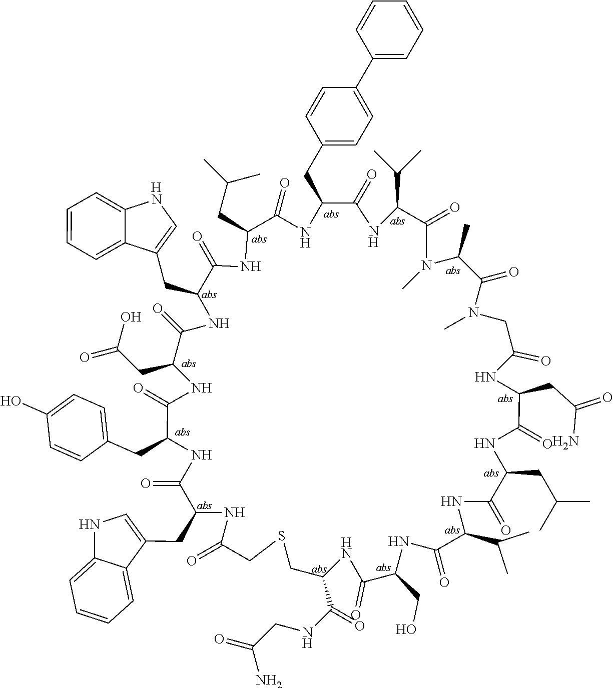

Example 1135 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 20 minutes, then a 5-minute hold at 100% B; Flow: 20 mL/min. Fractions containing the desired product were combined and dried via centrifugal evaporation. The yield of the product was 32.7 mg, and its estimated purity by LCMS analysis was 100%.
Analysis condition A: Retention time=1.56 min ESI-MS(+) m/z [M+2H]²⁺: 937.2.

### Preparation of Example 1136

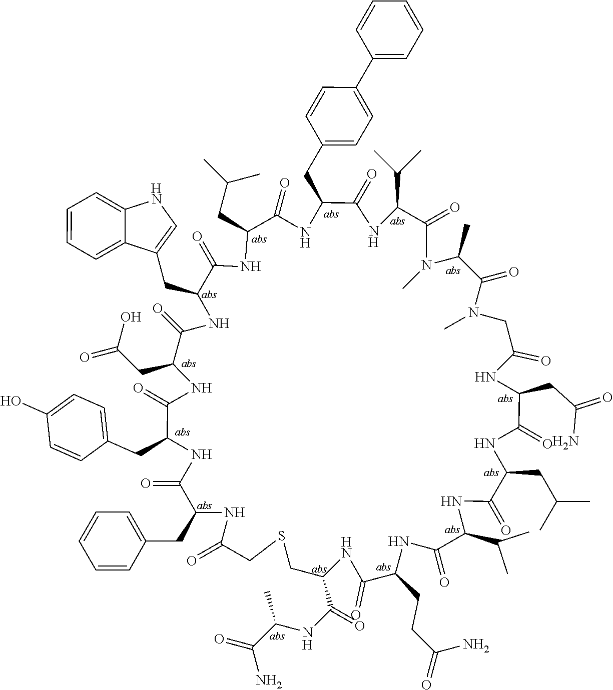

Example 1136 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, “Symphony Chloroacetic Anhydride coupling procedure”, “Global Deprotection Method A”, and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 20 minutes, then a 5-minute hold at 100% B; Flow: 20 mL/min. Fractions containing the desired product were combined and dried via centrifugal evaporation. The yield of the product was 15.5 mg, and its estimated purity by LCMS analysis was 97.6%.
Analysis condition A: Retention time=1.58 min; ESI-MS(+) m/z [M+2H]²⁺: 945.0.
Analysis condition B: Retention time=1.71 min; ESI-MS(+) m/z [M+2H]²⁺: 945.2.

### Preparation of Example 1137

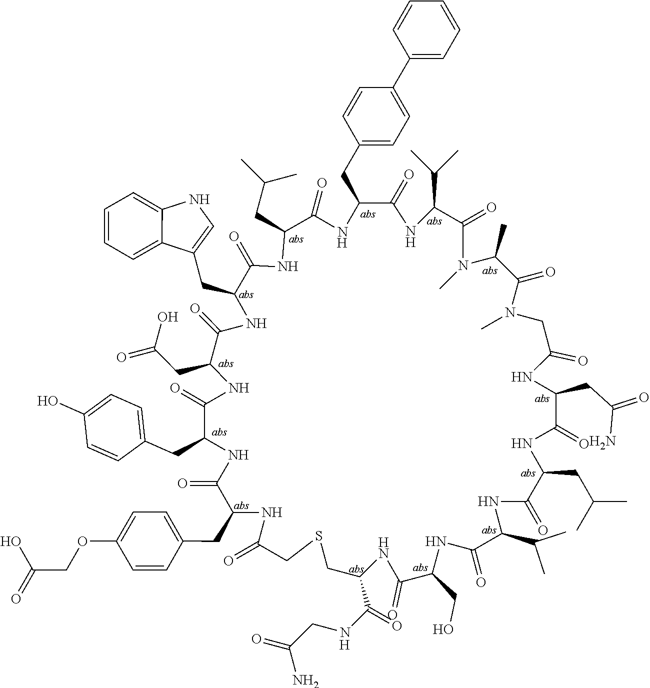

Example 1137 was prepared, using Sieber or Rink on a 50 μmol scale, following the general synthetic sequence described for the preparation of Example 1054, composed of the following general procedures: “Prelude Resin-swelling procedure”, “Prelude Single-coupling procedure”, “Symphony Resin-swelling procedure”, “Symphony Single-coupling procedure”, “Symphony Single-Coupling Pre-activation Procedure” was followed with Fmoc-Tyr(CH₂COOtBu)-OH; “Symphony Chloroacetic Anhydride coupling procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-55% B over 20 minutes, then a 5-minute hold at 100% B; Flow: 20 mL/min. Fractions containing the desired product were combined and dried via centrifugal evaporation. The yield of the product was 17.9 mg, and its estimated purity by LCMS analysis was 98.6%.
Analysis condition A: Retention time=1.33 min; ESI-MS(+) m/z [M+2H]²⁺: 955.2.
Analysis condition B: Retention time=1.64 min: ESI-MS(+) m/z [M+2H]²⁺: 955.1.

### Preparation of Example 1138

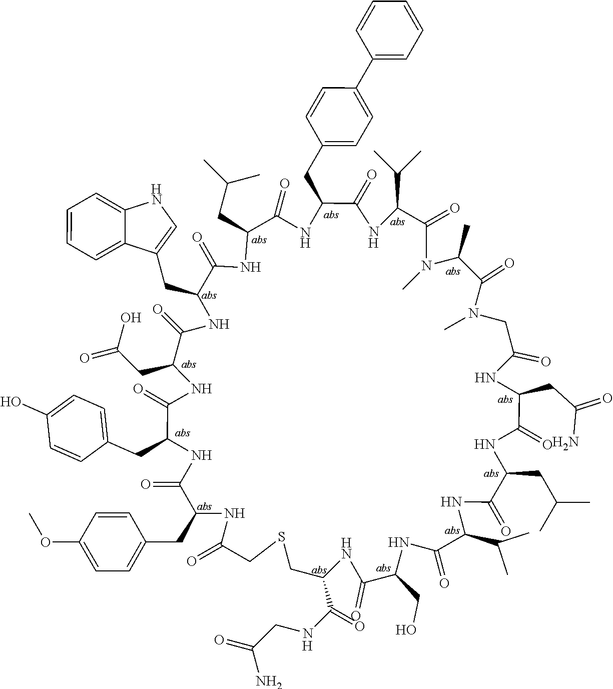

Example 1138 was prepared, using Sieber or Rink on a 50 μmol scale, following the general synthetic sequence described for the preparation of Example 1054 composed of the following general procedures: “Prelude Resin-swelling procedure”, “Prelude Single-coupling procedure”, “Symphony X Resin-swelling procedure”, “Symphony X Single-coupling procedure”, “Symphony X Single-Coupling Single-shot Procedure” was followed with Fmoc-Tyr(Me)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×150 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 20-70% B over 20 minutes, then a 2-minute hold at 100% B; Flow: 40 mL/min. Fractions containing the desired product were combined and dried via centrifugal evaporation. The yield of the product was 25.9 mg, and its estimated purity by LCMS analysis was 99.4%.
Analysis condition A: Retention time=1.58 min; ESI-MS(+) m/z [M+H]⁺: 1864.9.
Analysis condition B: Retention time=1.81 min: ESI-MS(+) m/z [M+2H]²⁺: 933.1.

### Preparation of Example 1139

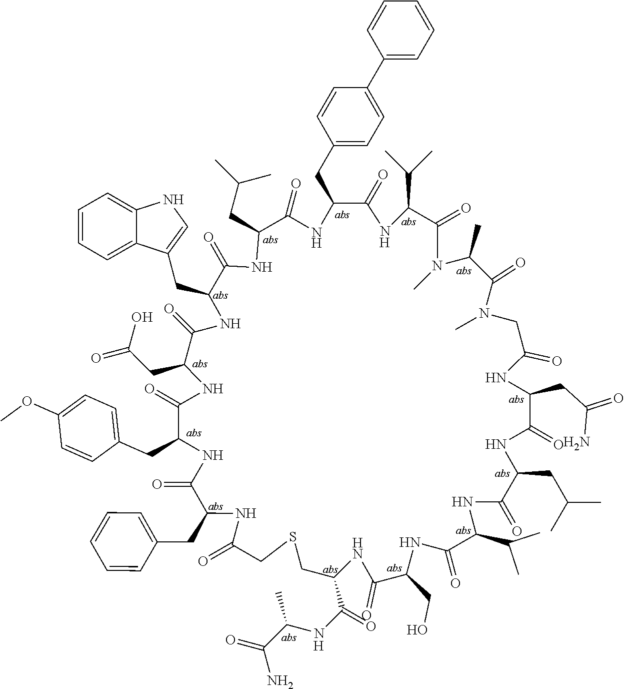

Example 1139 was prepared, using Sieber or Rink on a 50 μmol scale, following the general synthetic sequence described for the preparation of Example 1054, composed of the following general procedures: “Prelude Resin-swelling procedure”, “Prelude Single-coupling procedure”, “Symphony X Resin-swelling procedure”, “Symphony X Single-coupling procedure”, “Symphony X Single-Coupling Manual Addition Procedure B” was followed with Fmoc-Tyr(Me)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 30-70% B over 20 minutes, then a 5-minute hold at 100% B; Flow: 20 mL/min. Fractions containing the desired product were combined and dried via centrifugal evaporation. The yield of the product was 41.4 mg, and its estimated purity by LCMS analysis was 100%.
Analysis condition A: Retention time=1.73 min; ESI-MS(+) m/z [M+2H]²⁺: 931.1.
Analysis condition B: Retention time=1.86 min; ESI-MS(+) m/z [M+2H]²⁺: 931.9.

### Preparation of Example 1140

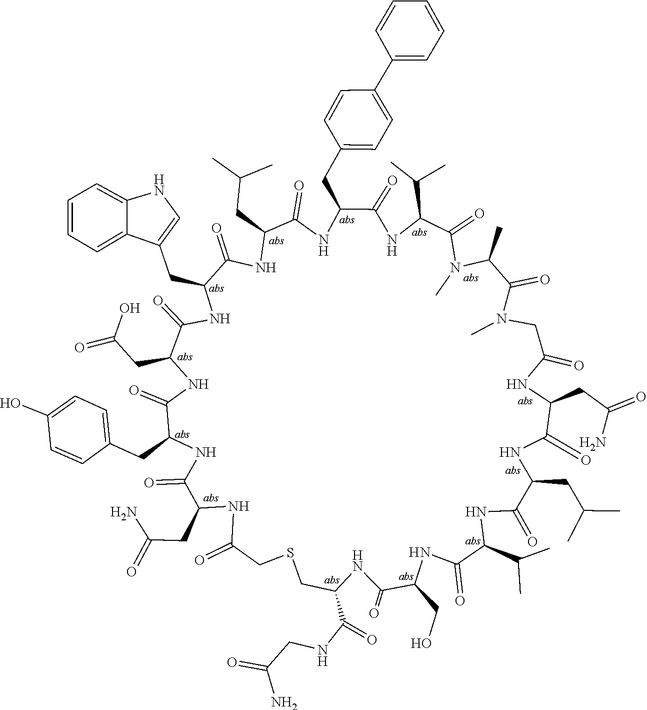 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1140 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 18-58% B over 25 minutes, then a 5-minute hold at 100% B; Flow: 20 mL/min. Fractions containing the desired product were combined and dried via centrifugal evaporation. The yield of the product was 31.1 mg, and its estimated purity by LCMS analysis was 90.7%.
Analysis condition A: Retention time=1.49 min; ESI-MS(+) m/z [M+H]⁺: 1800.9.
Analysis condition B: Retention time=1.63 min; ESI-MS(+) m/z [M+H]⁺: 1801.0.

### Preparation of Example 1141

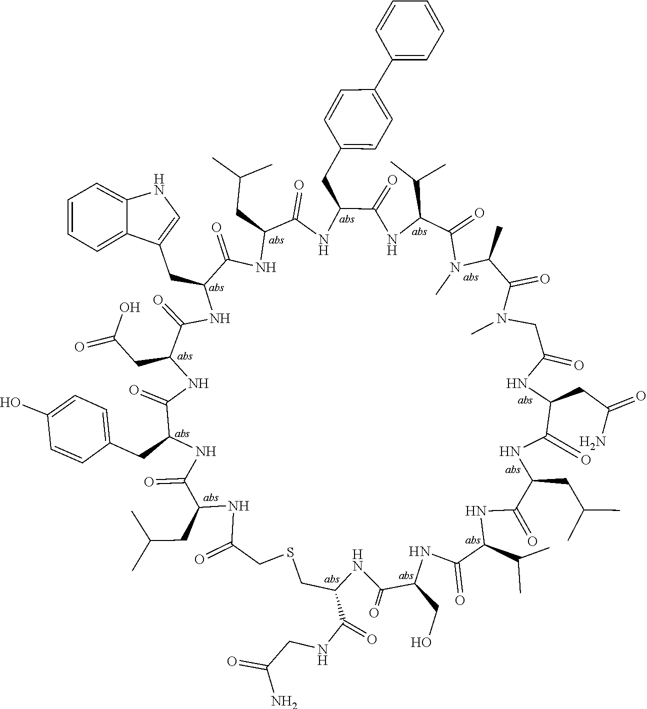 [SMILES: CC(C)C[C@@H]1NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(O)cc2)NC1=O]

Example 1141 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 20-60% B over 25 minutes, then a 5-minute hold at 100% B; Flow: 20 mL/min. Fractions containing the desired product were combined and dried via centrifugal evaporation. The yield of the product was 24.9 mg, and its estimated purity by LCMS analysis was 96.4%.
Analysis condition A: Retention time=1.74 min; ESI-MS(+) m/z [M+H]⁺: 1799.8.
Analysis condition B: Retention time=1.86 min; ESI-MS(+) m/z [M+H]⁺: 1799.8.

### Preparation of Example 1142

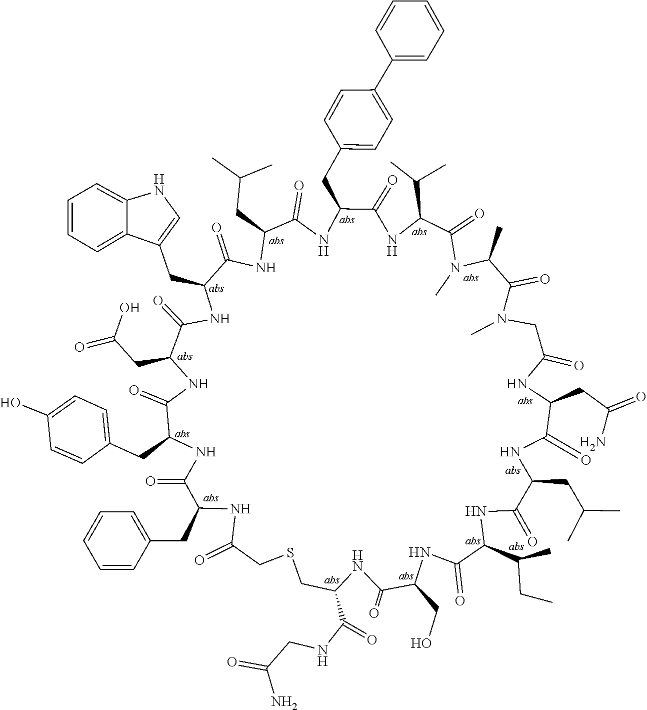 [SMILES: CC[C@H](C)[C@@H]1NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC1=O]

Example 1142 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-65% B over 20 minutes, then a 2-minute hold at 100% B; Flow: 45 mL/min. Fractions containing the desired product were combined and dried via centrifugal evaporation. The yield of the product was 37.6 mg, and its estimated purity by LCMS analysis was 98.2%.
Analysis condition A: Retention time=1.66 min; ESI-MS(+) m/z [M+2H]²⁺: 925.1.
Analysis condition B: Retention time=1.83 min; ESI-MS(+) m/z [M+H]⁺: 1848.1.

### Preparation of Example 1143

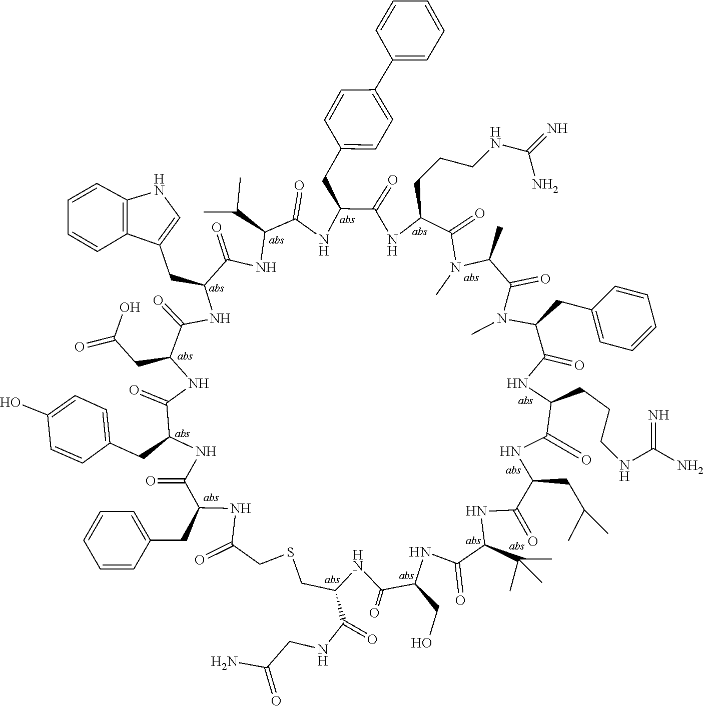 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)(C)C)NC1=O]

Example 1143 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, Single-Coupling Pre-Activation Procedure” for Fmoc-Tle-OH, “Symphony Chloroacetic Anhydride coupling procedure”, “Global Deprotection Method A”, and “Cyclization Method”.

The crude material was purified via preparative LC/MS with the following conditions: Column: XBridge C18, 30×150 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-65% B over 20 minutes, then a 2-minute hold at 100% B; Flow: 40 mL/min. Fractions containing the desired product were combined and dried via centrifugal evaporation. The yield of the product was 21.1 mg, and its estimated purity by LCMS analysis was 87.1%.
Analysis condition A: Retention time=2.0 min; ESI-MS(+) m/z [M+2H]²⁺: 1012.0.
Analysis condition B: Retention time=1.81 min; ESI-MS(+) m/z [M+2H]²⁺: 1012.5.

### Preparation of Example 1144

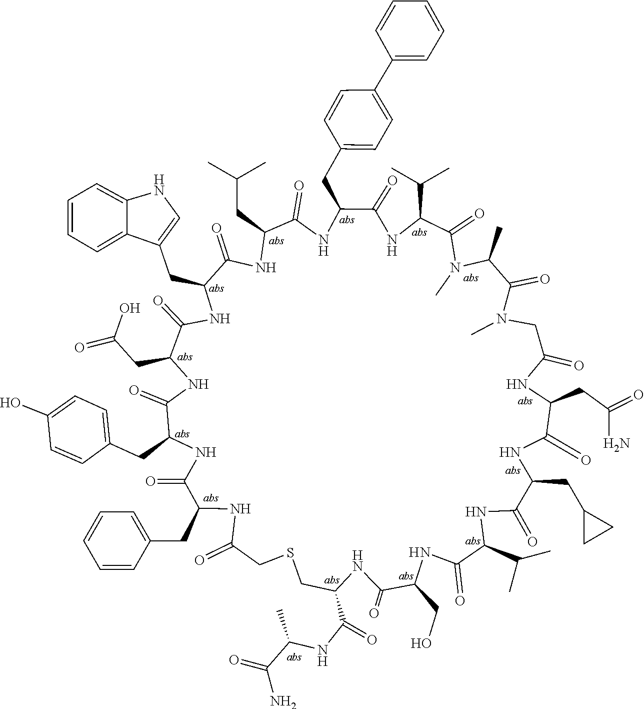 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC2CC2)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1144 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, Single-Coupling Pre-Activation Procedure” for Fmoc-cyclopropyl-Ala-OH, “Symphony Chloroacetic Anhydride coupling procedure”, “Global Deprotection Method A”, and “Cyclization Method”.

The crude material was purified via preparative LC/MS with the following conditions: Column: XBridge C18, 19×200 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 20-60% B over 20 minutes, then a 3-minute hold at 100% B; Flow: 20 mL/min. Fractions containing the desired product were combined and dried via centrifugal evaporation. The yield of the product was 14.7 mg, and its estimated purity by LCMS analysis was 97.1%.
Analysis condition A: Retention time=1.51 min; ESI-MS(+) m/z [M+2H]²⁺: 924.1.
Analysis condition B: Retention time=1.67 min; ESI-MS(+) m/z [M+2H]²⁺: 923.8.

### Preparation of Example 1145

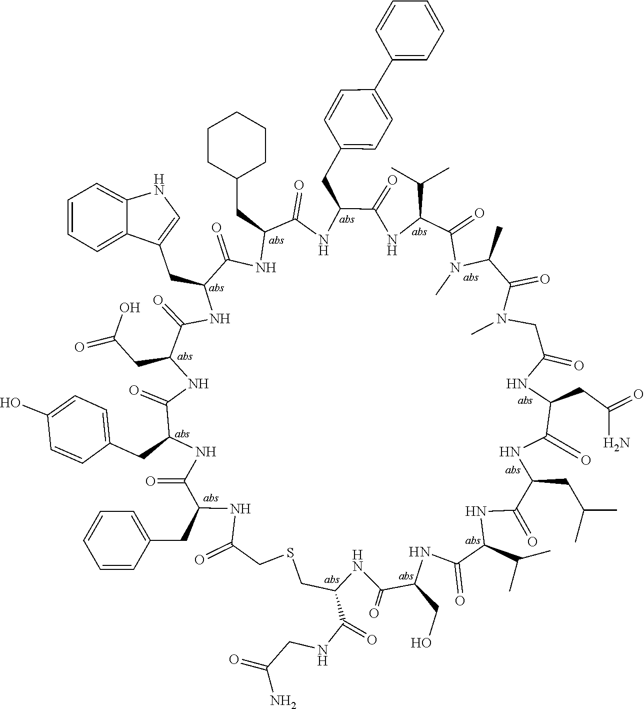 [SMILES: CC(C)C[C@@H]1NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC(=O)[C@H](CC2CCCCC2)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC1=O]

Example 1145 was prepared, using Sieber or Rink on a 50 μmol scale, following the general synthetic sequence described for the preparation of Example 1054, composed of the following general procedures: “Prelude Resin-swelling procedure”, “Prelude Single-coupling procedure”, “Symphony X Resin-swelling procedure”, “Symphony X Single-coupling procedure”, “Symphony X Single-Coupling Single-shot Procedure”; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-65% B over 20 minutes, then a 2-minute hold at 100% B; Flow: 45 mL/min. Fractions containing the desired product were combined and dried via centrifugal evaporation. The yield of the product was 26.8 mg, and its estimated purity by LCMS analysis was 98.2%.
Analysis condition A: Retention time=1.74 min; ESI-MS(+) m/z [M+H]⁺: 1875.1.
Analysis condition B: Retention time=1.92 min; ESI-MS(+) m/z [M+H]⁺: 1874.2.

### Preparation of Example 1146

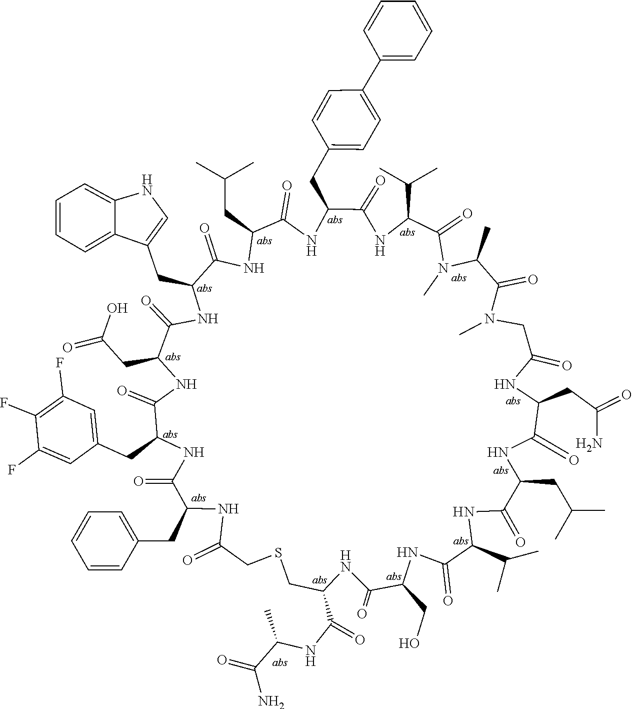 [SMILES: CC(C)C[C@@H]1NC(=O)[C@H](Cc2c[nH]c3ccccc23)NC(=O)[C@H](CC(=O)O)NC(=O)[C@H](Cc2cc(F)c(F)c(F)c2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1146 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, Single-Coupling Pre-Activation Procedure” for Fmoc-Phe (3,4,5-triF)—OH, “Symphony Chloroacetic Anhydride coupling procedure”, “Global Deprotection Method A”, and “Cyclization Method”.

The crude material was purified via preparative LC/MS with the following conditions: Column: XBridge C18, 19×200 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 35-75% B over 20 minutes, then a 3-minute hold at 100% B; Flow: 20 mL/min. Fractions containing the desired product were combined and dried via centrifugal evaporation. The yield of the product was 24.6 mg, and its estimated purity by LCMS analysis was 98.5%.
Analysis condition A: Retention time=1.85 min; ESI-MS(+) m/z [M+H]⁺: 1885.9.
Analysis condition B: Retention time=2.06 min; ESI-MS(+) m/z [M+H]⁺: 1885.9.

### Preparation of Example 1147

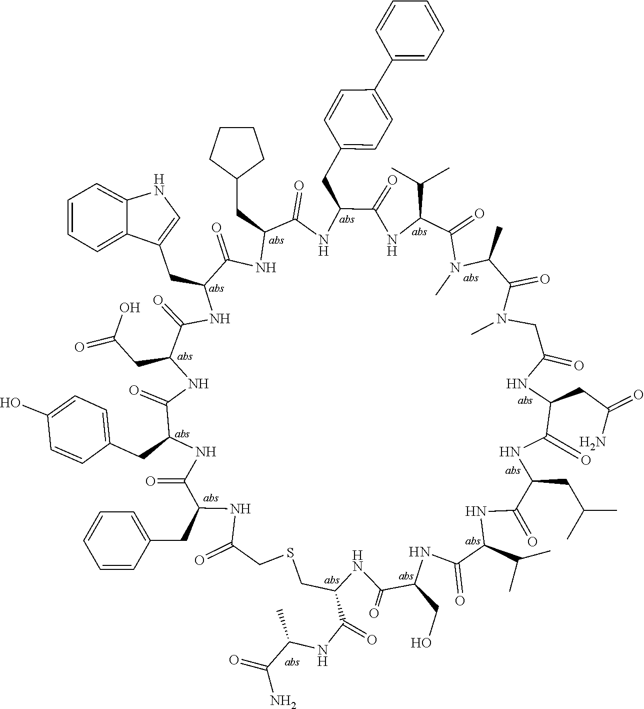 [SMILES: CC(C)C[C@@H]1NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC(=O)[C@H](CC2CCCC2)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC1=O]

Example 1147 was prepared, using Sieber or Rink on a 50 μmol scale, following the general synthetic sequence described for the preparation of Example 1054, composed of the following general procedures: “Prelude Resin-swelling procedure”, “Prelude Single-coupling procedure”, “Symphony X Resin-swelling procedure”, “Symphony X Single-coupling procedure”, “Symphony X Single-Coupling Single-shot Procedure”; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-65% B over 20 minutes, then a 2-minute hold at 100% B; Flow: 45 mL/min. Fractions containing the desired product were combined and dried via centrifugal evaporation. The yield of the product was 21.4 mg, and its estimated purity by LCMS analysis was 95.9%.
Analysis condition A: Retention time=1.72 min; ESI-MS(+) m/z [M+2H]²⁺: 938.0.
Analysis condition B: Retention time=1.85 min; ESI-MS(+) m/z [M+H]⁺: 1874.0.

### Preparation of Example 1148

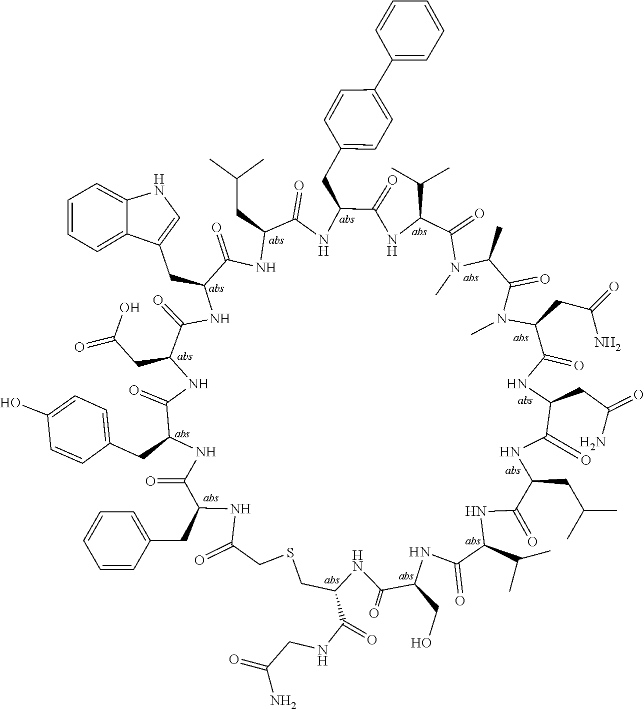 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CC(N)=O)NC(=O)[C@H](CC(N)=O)N(C)C(=O)[C@H](C)N(C)C(=O)[C@H](C(C)C)NC(=O)[C@H](Cc2ccc(-c3ccccc3)cc2)NC1=O]

Example 1148 was prepared, using Sieber or Rink on a 50 μmol scale, following the general synthetic sequence described for the preparation of Example 1054, composed of the following general procedures: “Prelude Resin-swelling procedure”, “Prelude Single-coupling procedure”, “Symphony Resin-swelling procedure”, “Symphony Single-coupling procedure” “Symphony Single-Coupling Pre-activation Procedure” was followed with Fmoc-NMe-Asn(Trt)-OH; “Symphony Chloroacetic Anhydride coupling procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 20 minutes, then a 5-minute hold at 100% B; Flow: 20 mL/min. Fractions containing the desired product were combined and dried via centrifugal evaporation. The yield of the product was 12.8 mg, and its estimated purity by LCMS analysis was 97.9%.
Analysis condition A: Retention time=1.51 min; ESI-MS(+) m/z [M+2H]²⁺: 946.1.
Analysis condition B: Retention time=1.68 min; ESI-MS(+) m/z [M+2H]²⁺: 946.5.

### Preparation of Example 1149

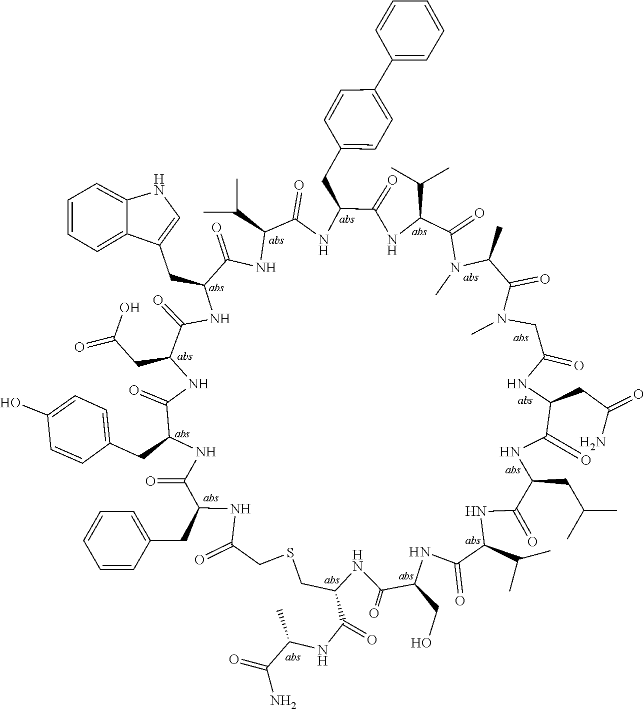 [SMILES: CC(C)C[C@@H]1NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC1=O]

Example 1149 was prepared, using Sieber or Rink on a 50 μmol scale, following the general synthetic sequence described for the preparation of Example 1054, composed of the following general procedures: “Prelude Resin-swelling procedure”, “Prelude Single-coupling procedure”, “Symphony X Resin-swelling procedure”, “Symphony X Single-coupling procedure”, “Symphony X Single-Coupling Single-shot Procedure”; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-65% B over 20 minutes, then a 2-minute hold at 100% B; Flow: 45 mL/min. Fractions containing the desired product were combined and dried via centrifugal evaporation. The yield of the product was 5.8 mg, and its estimated purity by LCMS analysis was 93.9%.
Analysis condition A: Retention time=1.5 min; ESI-MS(+) m % [M+2H]²⁺: 918.1.
Analysis condition B: Retention time=1.63 min; ESI-MS(+) m/z [M+2H]²⁺: 918.2.

### Preparation of Example 1150

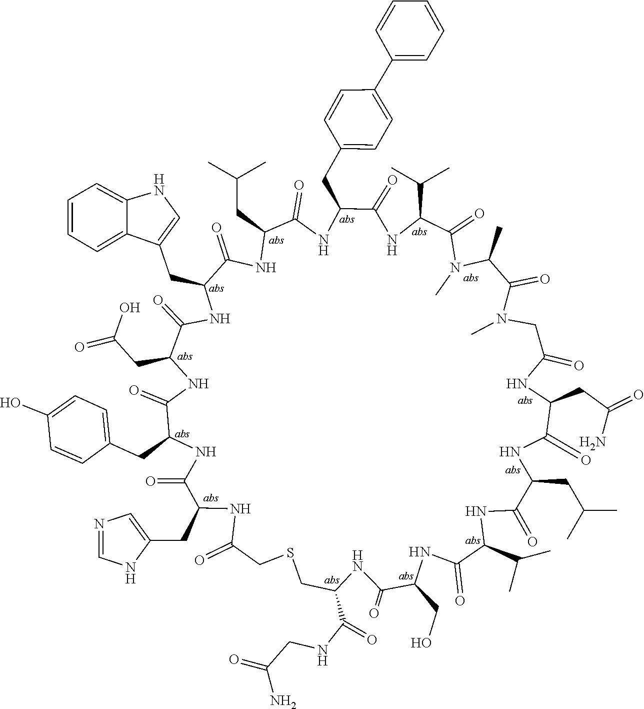 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2cnc[nH]2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1150 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 10-60% B over 20 minutes, then a 2-minute hold at 100% B; Flow: 45 mL/min. Fractions containing the desired product were combined and dried via centrifugal evaporation. The yield of the product was 24.7 mg, and its estimated purity by LCMS analysis was 94.2%.
Analysis condition A: Retention time=1.51 min; ESI-MS(+) m/z [M+H]⁺: 1823.8.
Analysis condition B: Retention time=1.64 min; ESI-MS(+) m/z [M+H]⁺: 1824.0.

### Preparation of Example 1151

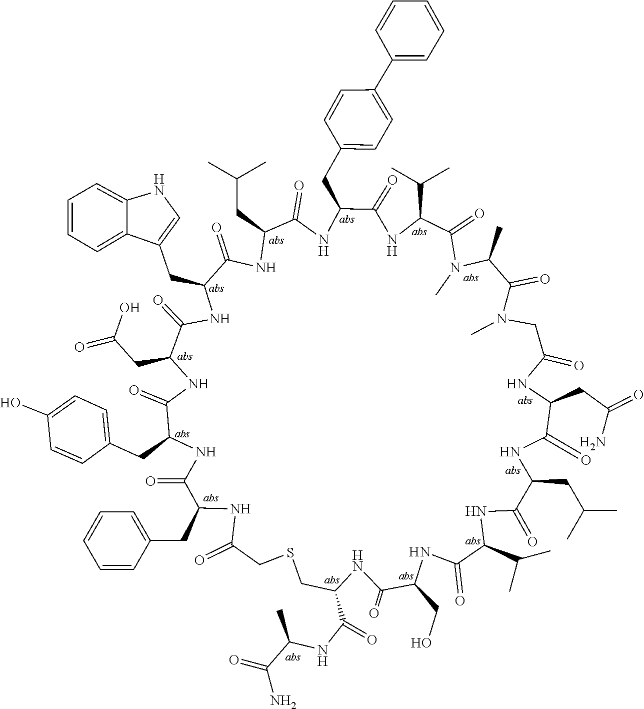 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@H](C)C(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1151 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, Single-Coupling Pre-Activation Procedure” for Fmoc-D-Ala-OH, “Symphony Chloroacetic Anhydride coupling procedure”, “Global Deprotection Method A”, and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 20 minutes, then a 5-minute hold at 100% B; Flow: 20 mL/min. Fractions containing the desired product were combined and dried via centrifugal evaporation. The yield of the product was 35 mg, and its estimated purity by LCMS analysis was 100%.
Analysis condition A: Retention time=1.63 min; ESI-MS(+) m/z [M+H]⁺: 1847.9.
Analysis condition B: Retention time=1.79 min; ESI-MS(+) m/z [M+H]⁺: 1848.3.

### Preparation of Example 1152

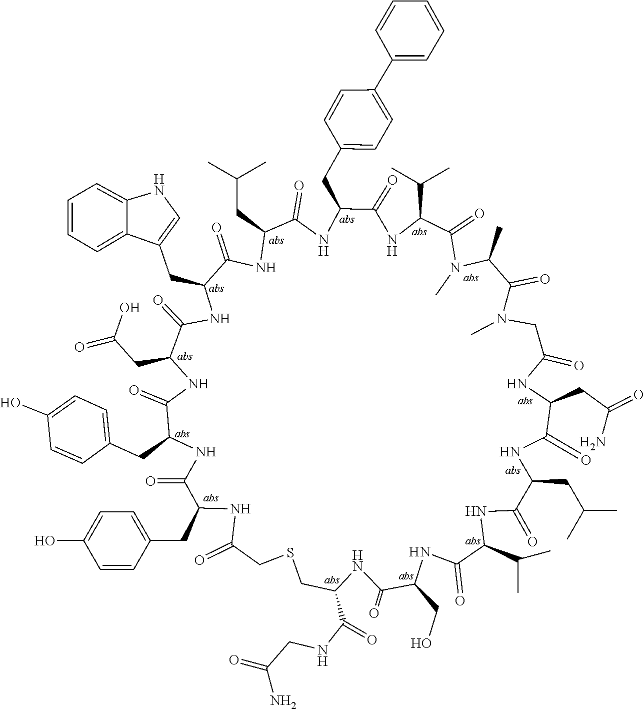 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccc(O)cc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1152 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 20 minutes, then a 5-minute hold at 100% B; Flow: 20 mL/min. Fractions containing the desired product were combined and dried via centrifugal evaporation. The yield of the product was 20.8 mg, and its estimated purity by LCMS analysis was 95.6%.
Analysis condition A: Retention time=1.47 min; ESI-MS(+) m/z [M+2H]²⁺: 926.0.
Analysis condition B: Retention time=1.65 min; ESI-MS(+) m/z [M+2H]²⁺: 925.9.

### Preparation of Example 1153

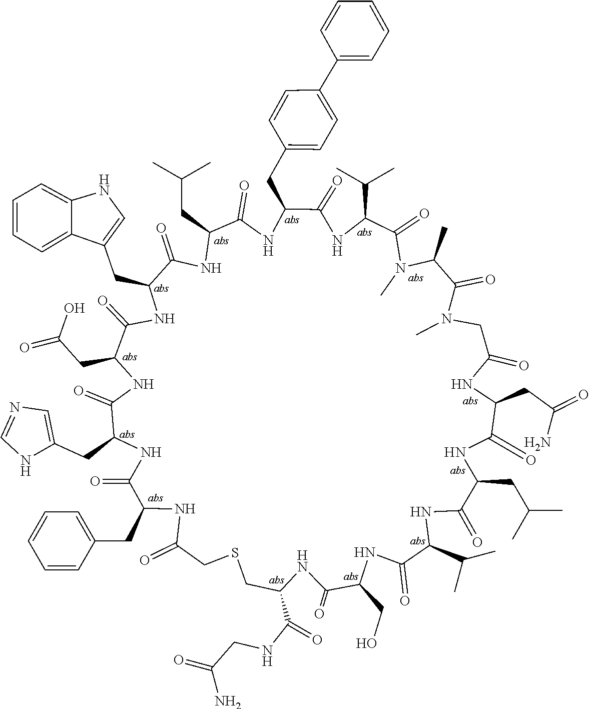 [SMILES: CC(C)C[C@@H]1NC(=O)[C@H](Cc2c[nH]c3ccccc23)NC(=O)[C@H](CC(=O)O)NC(=O)[C@H](Cc2cnc[nH]2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1153 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×150 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 10-60% B over 20 minutes, then a 2-minute hold at 100% B; Flow: 40 mL/min. Fractions containing the desired product were combined and dried via centrifugal evaporation. The yield of the product was 44.7 mg, and its estimated purity by LCMS analysis was 94.5%.
Analysis condition A: Retention time=1.54 min; ESI-MS(+) m/z [M+H]⁺: 1809.0.
Analysis condition B: Retention time=1.66 min: ESI-MS(+) m/z [M+H]⁺: 1809.0.

### Preparation of Example 1154

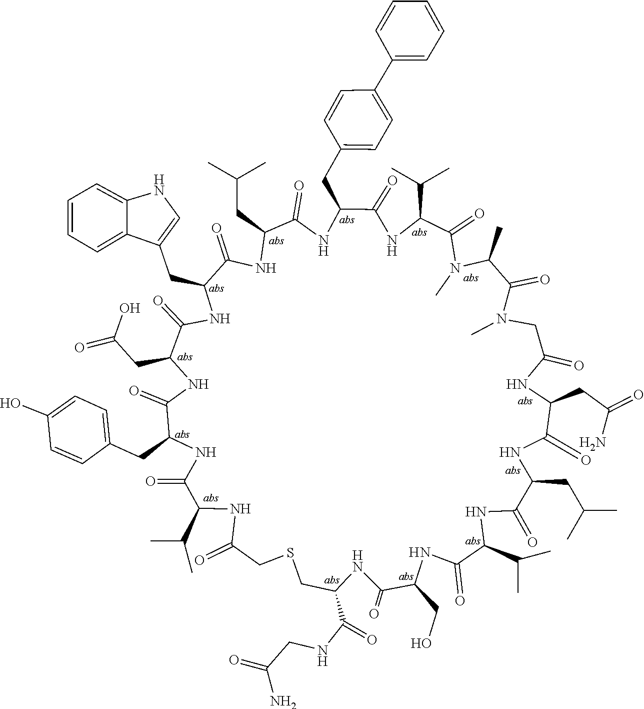 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](C(C)C)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1154 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 20 minutes, then a 5-minute hold at 100% B; Flow: 20 mL/min. Fractions containing the desired product were combined and dried via centrifugal evaporation. The yield of the product was 13.1 mg, and its estimated purity by LCMS analysis was 100%.
Analysis condition A: Retention time=1.49 min; ESI-MS(+) m/z [M+2H]²⁺: 894.1.
Analysis condition B: Retention time=1.64 min; ESI-MS(+) m/z [M+2H]²⁺: 894.2.

### Preparation of Example 1155

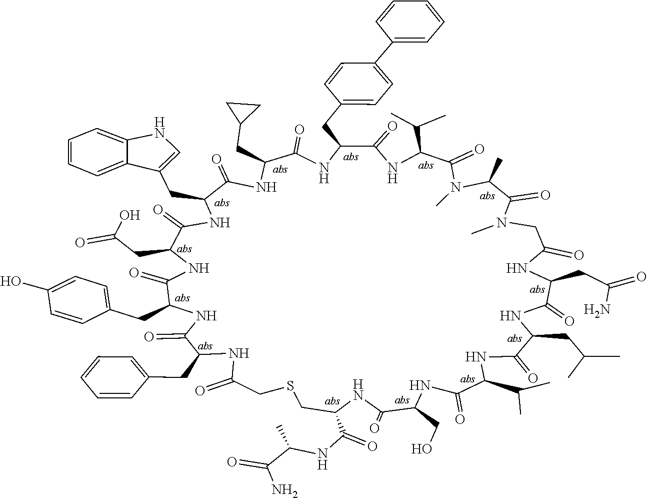 [SMILES: CC(C)C[C@@H]1NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC(=O)[C@H](CC2CC2)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC1=O]

Example 1155 was prepared, using Sieber or Rink on a 50 μmol scale, following the general synthetic sequence described for the preparation of Example 1054, composed of the following general procedures: “Prelude Resin-swelling procedure”, “Prelude Single-coupling procedure”, “Symphony X Resin-swelling procedure”, “Symphony X Single-coupling procedure”, “Symphony X Single-Coupling Single-shot Procedure”; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-65% B over 20 minutes, then a 2-minute hold at 100% B; Flow: 45 mL/min. Fractions containing the desired product were combined and dried via centrifugal evaporation. The yield of the product was 27.6 mg, and its estimated purity by LCMS analysis was 97.5%.
Analysis condition A: Retention time=1.59 min; ESI-MS(+) m/z [M+H]⁺: 1846.1.
Analysis condition B: Retention time=1.78 min; ESI-MS(+) m/z [M+2H]²⁺: 924.1.

### Preparation of Example 1156

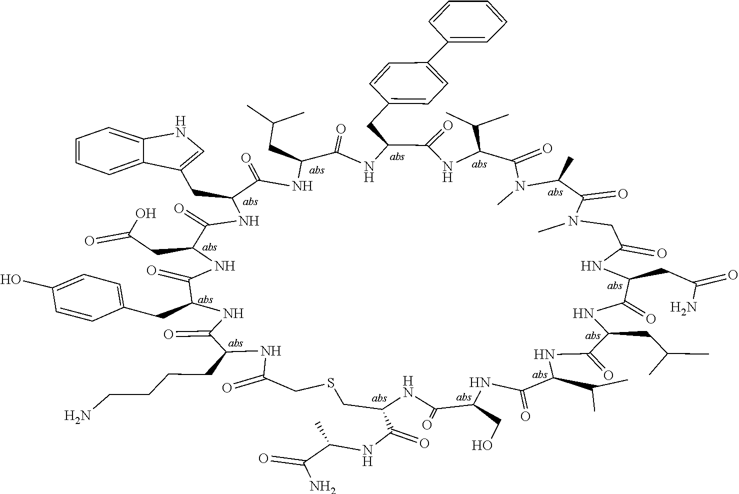 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](CCCCN)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1156 was prepared, using Sieber or Rink on a 50 μmol scale, following the general synthetic sequence described for the preparation of Example 1054, composed of the following general procedures: “Prelude Resin-swelling procedure”, “Prelude Single-coupling procedure”, “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-55% B over 20 minutes, then a 5-minute hold at 100% B; Flow: 20 mL/min. Fractions containing the desired product were combined and dried via centrifugal evaporation. The yield of the product was 21.5 mg, and its estimated purity by LCMS analysis was 98.5%.
Analysis condition A: Retention time=1.55 min; ESI-MS(+) m/z [M−H]⁻: 1814.7.
Analysis condition B: Retention time=1.61 min; ESI-MS(+) m/z [M+H]⁺: 1816.0

### Preparation of Example 1157

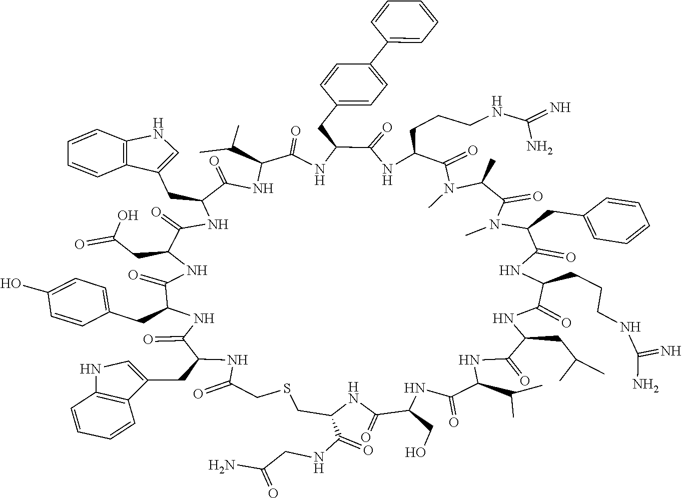 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2c[nH]c3ccccc23)NC(=O)CSC[C@@H](C(=O)NCC(N)=O)NC(=O)[C@H](CO)NC(=O)[C@H](C(C)C)NC1=O]

Example 1157 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×150 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 15-65% B over 20 minutes, then a 2-minute hold at 100% B; Flow: 40 mL/min. Fractions containing the desired product were combined and dried via centrifugal evaporation. The yield of the product was 27.9 mg, and its estimated purity by LCMS analysis was 100%.
Analysis condition A: Retention time=1.82 min; ESI-MS(+) m/z [M+2H]²⁺: 1024.9.
Analysis condition B: Retention time=1.81 min; ESI-MS(+) m/z [M+2H]²⁺: 1024.9.

### Preparation of Example 1158

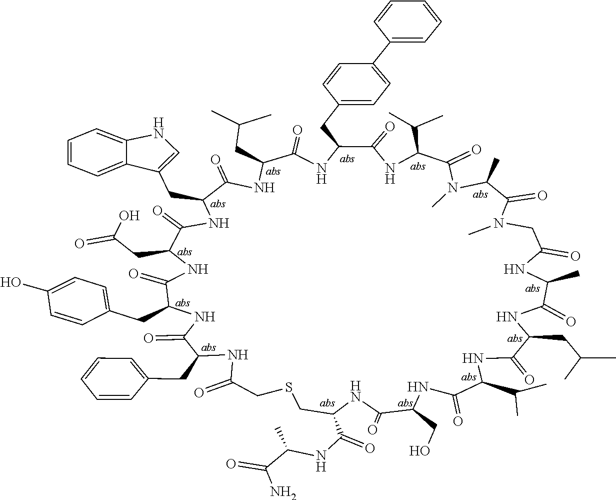 [SMILES: CC(C)C[C@@H]1NC(=O)[C@H](C)NC(=O)CN(C)C(=O)[C@H](C)N(C)C(=O)[C@H](C(C)C)NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC1=O]

Example 1158 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×150 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 20-70% B over 20 minutes, then a 2-minute hold at 100% B; Flow: 40 mL/min. Fractions containing the desired product were combined and dried via centrifugal evaporation. The yield of the product was 16.4 mg, and its estimated purity by LCMS analysis was 95.3%.
Analysis condition A: Retention time=1.62 min; ESI-MS(+) m/z [M+H]⁺: 1805.1.
Analysis condition B: Retention time=1.82 min; ESI-MS(+) m/z [M+H]⁺: 1806.1.

### Preparation of Example 1159

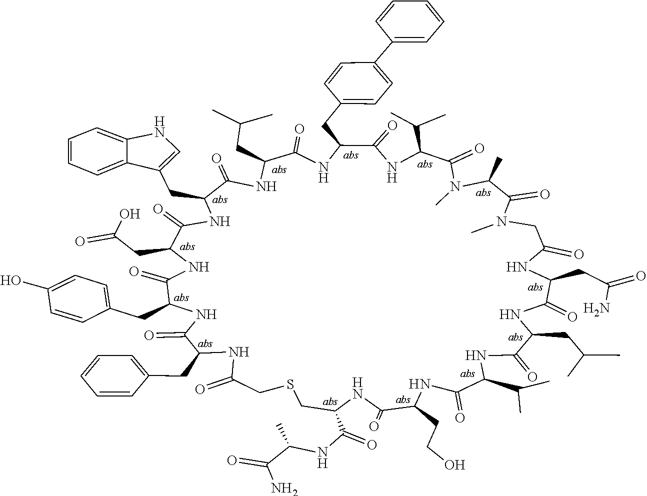 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CCO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1159 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, Single-Coupling Pre-Activation Procedure” for Fmoc-Home-Ser(tBu)-OH, “Symphony Chloroacetic Anhydride coupling procedure”, “Global Deprotection Method A”, and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 20 minutes, then a 5-minute hold at 100% B; Flow: 20 mL/min. Fractions containing the desired product were combined and dried via centrifugal evaporation. The yield of the product was 12.3 mg, and its estimated purity by LCMS analysis was 95.3%.
Analysis condition A: Retention time=1.62 min; ESI-MS(+) m/z [M+H]⁺: 1862.2.
Analysis condition B: Retention time=1.83 min; ESI-MS(+) m/z [M+H]⁺: 1862.0.

### Preparation of Example 1160

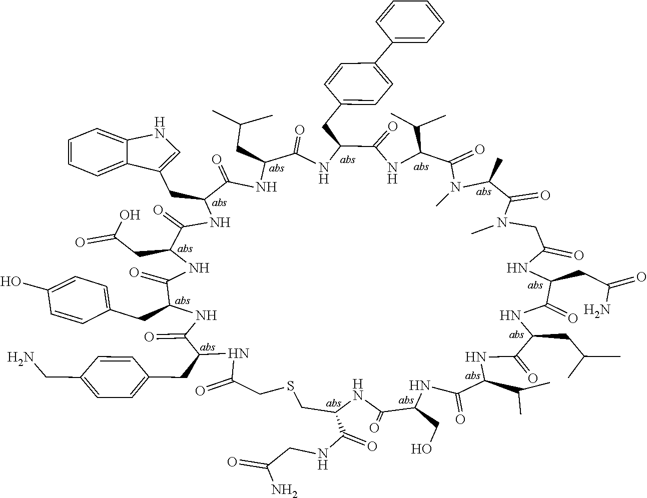 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccc(CN)cc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1160 was prepared, using Sieber or Rink on a 50 μmol scale, following the general synthetic sequence described for the preparation of Example 1054, composed of the following general procedures: “Prelude Resin-swelling procedure”, “Prelude Single-coupling procedure”, “Symphony Resin-swelling procedure”, “Symphony Single-coupling procedure” “Symphony Single-Coupling Pre-activation Procedure” was followed with Fmoc-Phe(4-CH₂NHBoc)-OH; “Symphony Chloroacetic Anhydride coupling procedure”, “Global Deprotection Method A”, “Cyclization Method A”.

The crude material was purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 10-60% B over 20 minutes, then a 2-minute hold at 100% B; Flow: 45 mL/min. Fractions containing the desired product were combined and dried via centrifugal evaporation. The yield of the product was 11.8 mg, and its estimated purity by LCMS analysis was 98.8%.
Analysis condition A: Retention time=1.48 min; ESI-MS(+) m/z [M+3H]³⁺: 932.0.
Analysis condition B: Retention time=1.56 min; ESI-MS(+) m/z [M+3H]³⁺: 622.0.

### Preparation of Example 1161

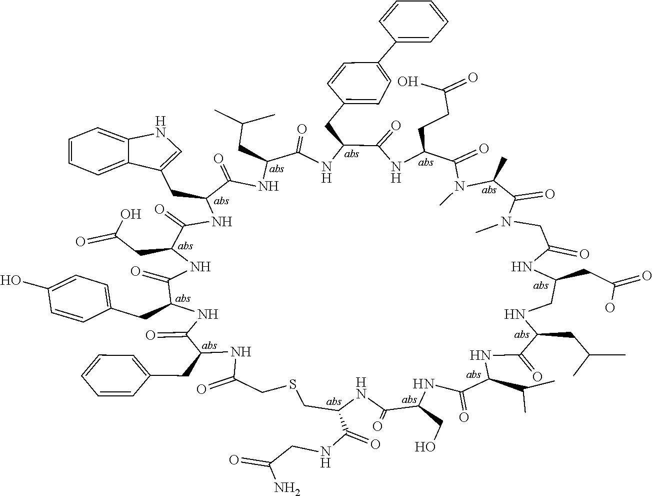 [SMILES: CC(C)C[C@@H]1NC[C@H](CC(=O)O)NC(=O)CN(C)C(=O)[C@H](C)N(C)C(=O)[C@H](CCC(=O)O)NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC1=O]

Example 1161 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”,

“Symphony X Chloroacetic Anhydride coupling procedure”, “Symphony X Final rinse and dry procedure”, “Global Deprotection Method B”, “Cyclization Method A”.

The crude material was purified via preparative LC/MS with the following conditions: Column: XBridge C18, 30×150 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-55% B over 20 minutes, then a 5-minute hold at 100% B; Flow: 20 mL/min. Fractions containing the desired product were combined and dried via centrifugal evaporation. The yield of the product was 37.5 mg, and its estimated purity by LCMS analysis was 89.2%.
Analysis condition A: Retention time=1.46 min; ESI-MS(+) m/z [M+H]⁺: 1864.9.
Analysis condition B: Retention time=1.58 min; ESI-MS(+) m/z [M+2H]²⁺: 933.1.

### Preparation of Example 1162

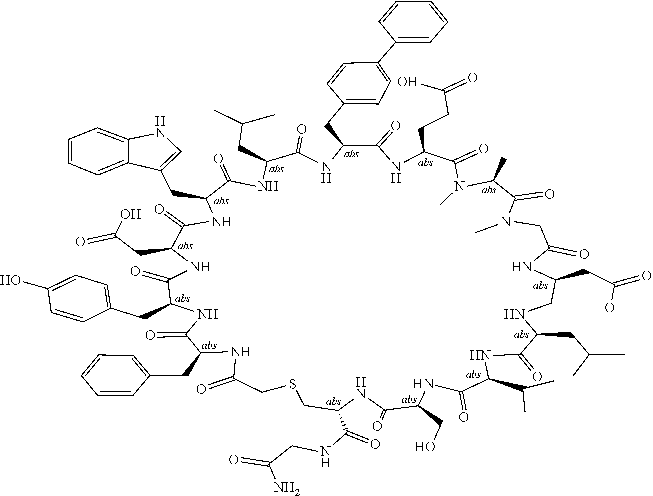 [SMILES: CC(C)C[C@@H]1NC[C@H](CC(=O)O)NC(=O)CN(C)C(=O)[C@H](C)N(C)C(=O)[C@H](CCC(=O)O)NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC1=O]

Example 1162 was prepared, using Sieber or Rink on a −1 μmol scale, following the general synthetic sequence described for the preparation of Example 1000 composed of the following general procedures: “Symphony Resin-swelling procedure”, “Symphony Single-coupling procedure”, Single-Coupling Pre-Activation Procedure” for Fmoc-Phe (4-F)—OH, “Symphony Chloroacetic Anhydride coupling procedure”, “Global Deprotection Method A”, and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 20 minutes, then a 5-minute hold at 100% B; Flow: 20 mL/min. Fractions containing the desired product were combined and dried via centrifugal evaporation. The yield of the product was 11.4 mg, and its estimated purity by LCMS analysis was 99.3%.
Analysis condition A: Retention time=1.78 min; ESI-MS(+) m/z [M+2H]²⁺: 925.3.
Analysis condition B: Retention time=1.88 min; ESI-MS(+) m/z [M+2H]²⁺: 926.2.

### Preparation of Example 1163

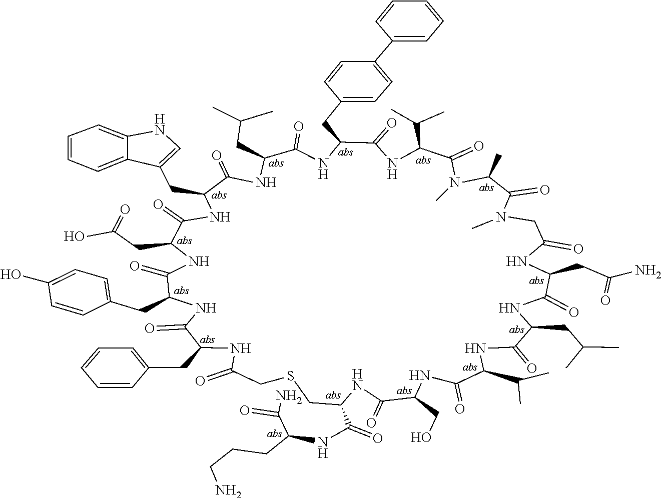

Example 1163 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, Single-Coupling Pre-Activation Procedure” for Fmoc-Orn-OH, “Symphony Chloroacetic Anhydride coupling procedure”, “Global Deprotection Method A”, and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 20 minutes, then a 5-minute hold at 100% B; Flow: 20 mL/min. Fractions containing the desired product were combined and dried via centrifugal evaporation. The yield of the product was 12.2 mg, and its estimated purity by LCMS analysis was 100%.
Analysis condition A: Retention time=1.64 min; ESI-MS(+) m/z [M+H]⁺: 1892.0.
Analysis condition B: Retention time=1.74 min; ESI-MS(+) m/z [M+Na]⁺: 1914.3.

### Preparation of Example 1164

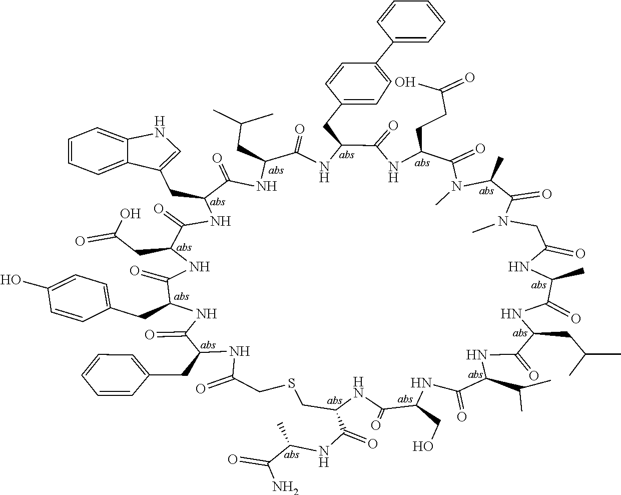 [SMILES: CC(C)C[C@@H]1NC(=O)[C@H](C)NC(=O)CN(C)C(=O)[C@H](C)N(C)C(=O)[C@H](CCC(=O)O)NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC1=O]

Example 1164 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 20 minutes, then a 5-minute hold at 100% B; Flow: 20 mL/min. Fractions containing the desired product were combined and dried via centrifugal evaporation. The yield of the product was 22.5 mg, and its estimated purity by LCMS analysis was 92.3%.
Analysis condition A: Retention time=1.53 min; ESI-MS(+) m/z [M+2H]²⁺: 918.1.
Analysis condition B: Retention time=1.8 min; ESI-MS(+) m/z [M+H]⁺: 1836.2.

### Preparation of Example 1165

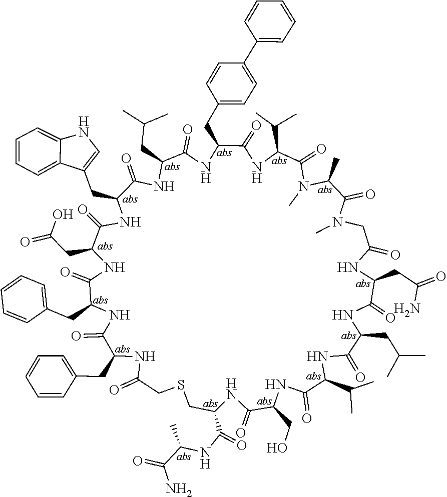 [SMILES: CC(C)C[C@@H]1NC(=O)[C@H](Cc2c[nH]c3ccccc23)NC(=O)[C@H](CC(=O)O)NC(=O)[C@H](Cc2ccccc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1165 was prepared, using Sieber or Rink on a 50 μmol scale, following the general synthetic sequence described for the preparation of Example 1054, composed of the following general procedures: “Prelude Resin-swelling procedure”, “Prelude Single-coupling procedure”, “Symphony X Resin-swelling procedure”, “Symphony X Single-coupling procedure”, “Symphony X Single-Coupling Single-shot Procedure”; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 30-70% B over 20 minutes, then a 4-minute hold at 100% B; Flow: 20 mL/min. Fractions containing the desired product were combined and dried via centrifugal evaporation. The yield of the product was 8.7 mg, and its estimated purity by LCMS analysis was 89.8%.
Analysis condition A: Retention time=1.76 min; ESI-MS(+) m/z [M+2H]²⁺: 916.1.
Analysis condition B: Retention time=1.92 min; ESI-MS(+) m/z [M+2H]²⁺: 916.1.

### Preparation of Example 1166

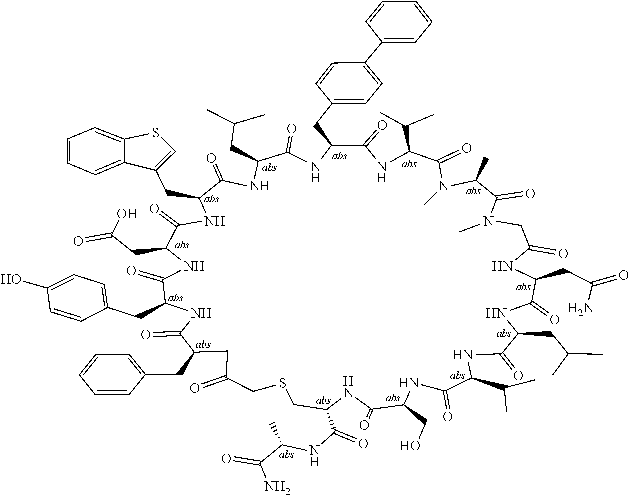 [SMILES: CC(C)C[C@@H]1NC(=O)[C@H](Cc2csc3ccccc23)NC(=O)[C@H](CC(=O)O)NC(=O)[C@H](Cc2ccc(O)cc2)NC(=O)[C@H](Cc2ccccc2)CC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1166 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”,

“Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×150 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 20-70% B over 20 minutes, then a 2-minute hold at 100% B; Flow: 40 mL/min. Fractions containing the desired product were combined and dried via centrifugal evaporation. The yield of the product was 22.8 mg, and its estimated purity by LCMS analysis was 98.7%.
Analysis condition A: Retention time=1.68 min; ESI-MS(+) m/z [M+H]⁺: 1864.9.
Analysis condition B: Retention time=1.89 min: ESI-MS(+) m/z [M+2H]²⁺: 933.1.

### Preparation of Example 1167

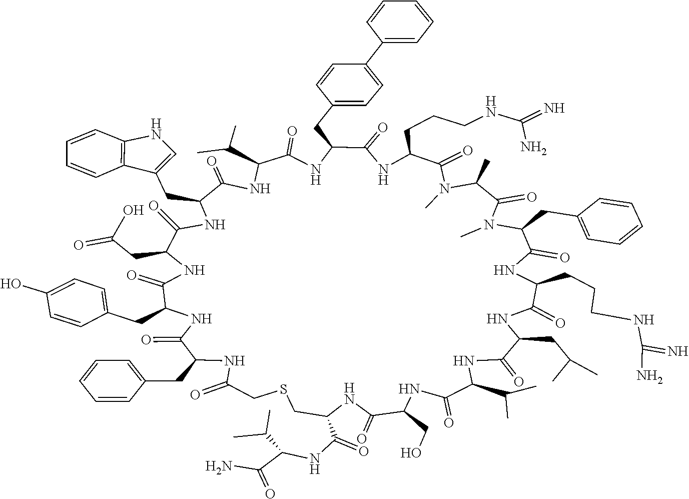 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@H](C(N)=O)C(C)C)NC(=O)[C@H](CO)NC(=O)[C@H](C(C)C)NC1=O]

Example 1167 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×150 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 15-65% B over 20 minutes, then a 2-minute hold at 100% B; Flow: 40 mL/min. Fractions containing the desired product were combined and dried via centrifugal evaporation. The yield of the product was 11.2 mg, and its estimated purity by LCMS analysis was 90.6%.
Analysis condition A: Retention time=1.86 min; ESI-MS(+) m/z [M+2H]²⁺: 1026.1.
Analysis condition B: Retention time=1.72 min; ESI-MS(+) m/z [M+2H]²⁺: 1026.3.

### Preparation of Example 1168

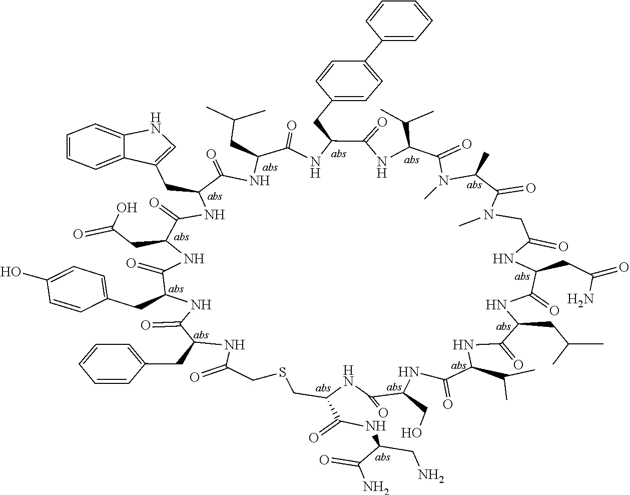 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](CN)C(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1168 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, Single-Coupling Pre-Activation Procedure” for Fmoc-Dap(Boc)-OH, “Symphony Chloroacetic Anhydride coupling procedure”, “Global Deprotection Method A”, and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 20 minutes, then a 5-minute hold at 100% B; Flow: 20 mL/min. Fractions containing the desired product were combined and dried via centrifugal evaporation. The yield of the product was 17.2 mg, and its estimated purity by LCMS analysis was 98.1%.
Analysis condition A: Retention time=1.66 min; ESI-MS(+) m/z [M+H]⁺: 1862.9.
Analysis condition B: Retention time=1.73 min; ESI-MS(+) m/z [M+H]⁺: 1863.1.

### Preparation of Example 1169

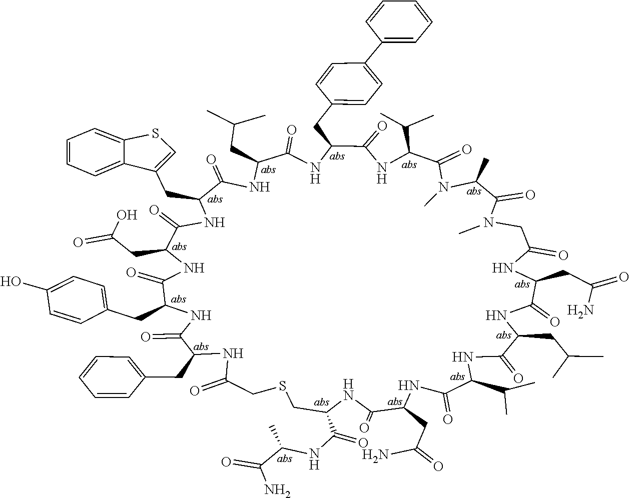 [SMILES: CC(C)C[C@@H]1NC(=O)[C@H](Cc2csc3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CC(N)=O)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1169 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, “Symphony Chloroacetic Anhydride coupling procedure”, “Global Deprotection Method A”, and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 20 minutes, then a 5-minute hold at 100% B; Flow: 20 mL/min. Fractions containing the desired product were combined and dried via centrifugal evaporation. The yield of the product was 9.1 mg, and its estimated purity by LCMS analysis was 94.1%.
Analysis condition A: Retention time=1.62 min; ESI-MS(+) m/z [M+2H]²⁺: 938.2.
Analysis condition B: Retention time=1.82 min; ESI-MS(+) m/z [M+H]⁺: 1874.9.

### Preparation of Example 1170

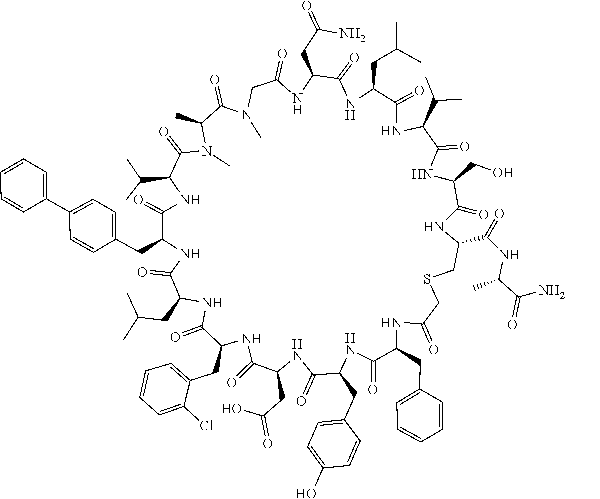

Example 1170 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”,

“Symphony X Single-Coupling Manual Addition Procedure B” was followed with Fmoc-Phe(2-Cl)—O H; “Symphony X(Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 20-60% B over 20 minutes, then a 5-minute hold at 100% B; Flow: 20 mL/min. Fractions containing the desired product were combined and dried via centrifugal evaporation. The yield of the product was 14.6 mg, and its estimated purity by LCMS analysis was 96.3%.
Analysis condition A: Retention time=1.6 min; ESI-MS(+) m/z [M+2H]²⁺: 922.0.
Analysis condition B: Retention time=1.79 min: ESI-MS(+) m/z [M+2H]²⁺: 922.1.

### Preparation of Example 1171

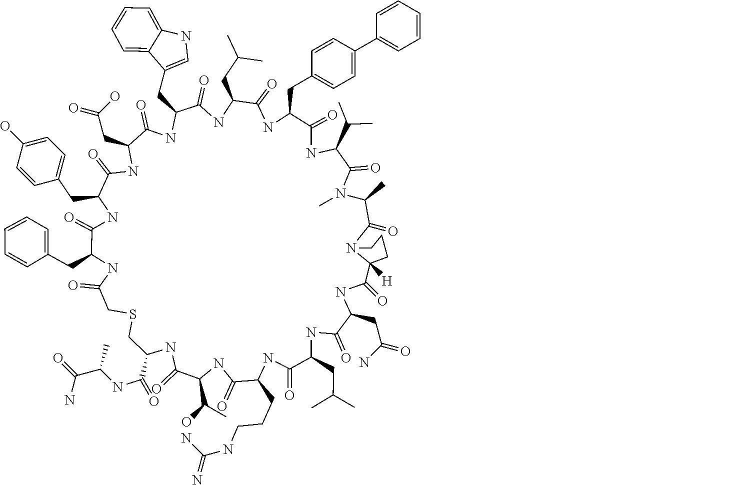

Example 1171 was prepared, using Sieber or Rink on a 50 μmol scale, following the general synthetic sequence described for the preparation of Example 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 25-65% B over 25 minutes, then a 5-minute hold at 100% Flow: 20 mL/min. Fractions containing the desired product were combined and dried via centrifugal evaporation. The yield of the product was 23.4 mg, and its estimated purity by LCMS analysis was 98.7%.
Analysis condition A: Retention time=1.7 min; ESI-MS(+) m/z [M+2H]²⁺: 973.3.
Analysis condition B: Retention time=1.78 min; ESI-MS(+) m/z [M+2H]²⁺: 973.3.

### Preparation of Example 1172

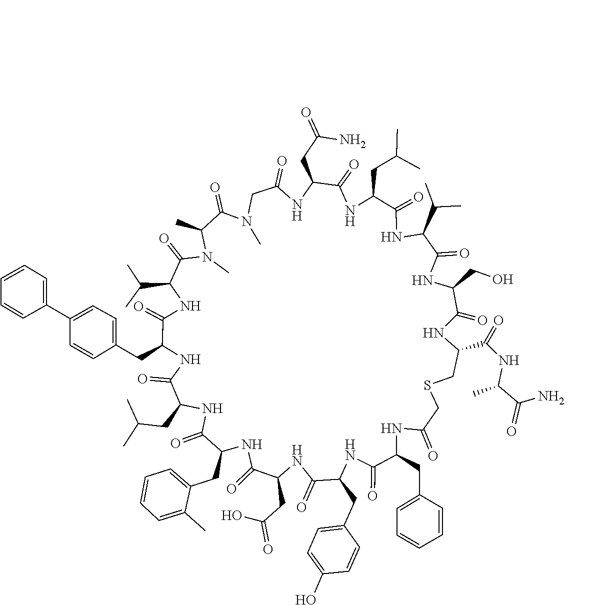

Example 1172 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, Single-Coupling Pre-Activation Procedure” for Fmoc-Phe (2-Me)-OH, “Symphony Chloroacetic Anhydride coupling procedure”, “Global Deprotection Method A”, and “Cyclization Method”.

The crude material was purified via preparative LC/MS with the following conditions: Column: XBridge C18, 19×200 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 30-70% B over 20 minutes, then a 3-minute hold at 100% B; Flow: 20 mL/min. Fractions containing the desired product were combined and dried via centrifugal evaporation. The yield of the product was 26.8 mg, and its estimated purity by LCMS analysis was 95.2%.
Analysis condition A: Retention time=1.66 min; ESI-MS(+) m/z [M+H]⁺: 1822.9.
Analysis condition B: Retention time=1.89 min; ESI-MS(+) m/z [M+H]⁺: 1823.1.

### Preparation of Example 1173

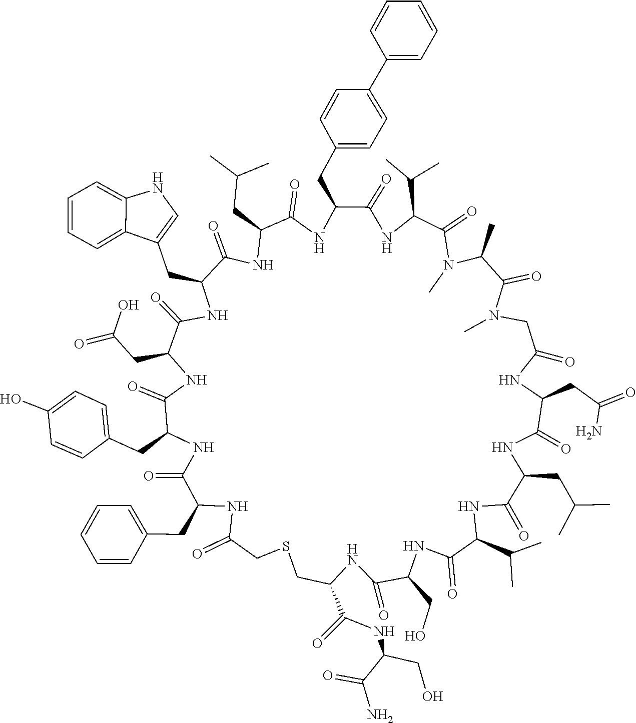 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](CO)C(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1173 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, “Symphony Chloroacetic Anhydride coupling procedure”, “Global Deprotection Method A”, and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 20 minutes, then a 5-minute hold at 100% B; Flow: 20 mL/min. Fractions containing the desired product were combined and dried via centrifugal evaporation. The yield of the product was 29.9 mg, and its estimated purity by LCMS analysis was 98.8%.
Analysis condition A: Retention time=1.61 min; ESI-MS(+) m/z [M+H]⁺: 1864.2.
Analysis condition B: Retention time=1.75 min; ESI-MS(+) m/z [M+H]⁺: 1864.3.

### Preparation of Example 1174

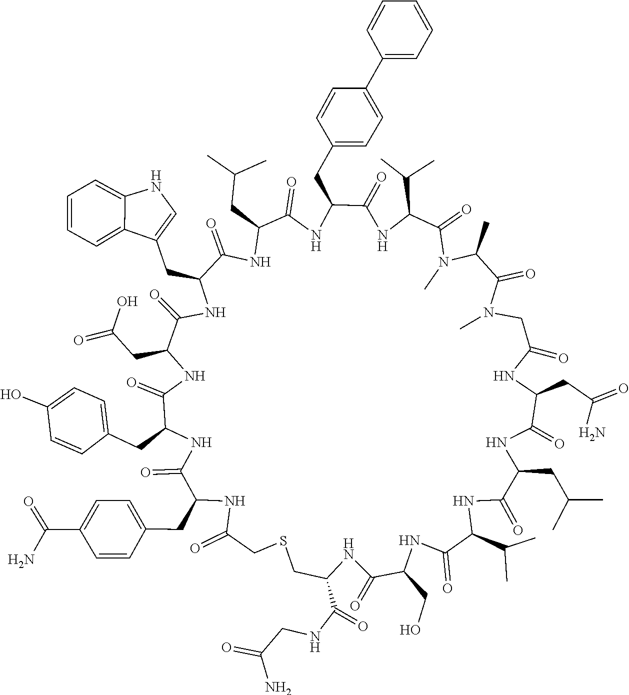 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccc(C(N)=O)cc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1174 was prepared, using Sieber or Rink on a 50 μmol scale, following the general synthetic sequence described for the preparation of Example 1054, composed of the following general procedures: “Prelude Resin-swelling procedure”, “Prelude Single-coupling procedure”, “Symphony Resin-swelling procedure”, “Symphony Single-coupling procedure” “Symphony Single-Coupling Pre-activation Procedure” was followed with Fmoc-Phe(4-CONH₂)—OH; “Symphony Chloroacetic Anhydride coupling procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-60% B over 20 minutes, then a 5-minute hold at 100% B; Flow: 20 mL/min. Fractions containing the desired product were combined and dried via centrifugal evaporation. The yield of the product was 28.1 mg, and its estimated purity by LCMS analysis was 97.9%.
Analysis condition A: Retention time=1.52 min; ESI-MS(+) m/z [M+H]⁺: 1877.0.
Analysis condition B: Retention time=1.66 min; ESI-MS(+) m/z [M+H]⁺: 1876.9.

### Preparation of Example 1175

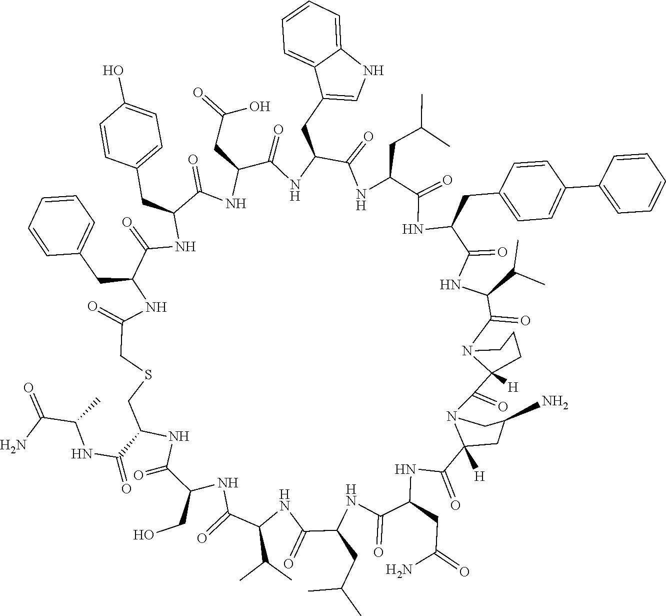

Example 1175 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, Single-Coupling Pre-Activation Procedure” for Fmoc-D-Pro(4-NHBoc)-OH, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 15-100% B over 19 minutes, then a 5-minute hold at 100% B; Flow: 20 mL/min. Fractions containing the desired product were combined and dried via centrifugal evaporation. The yield of the product was 42.4 mg, and its estimated purity by LCMS analysis was 95.5%.
Analysis condition A: Retention time=1.69 min; ESI-MS(+) m/z [M+2H]²⁺: 1900.9.
Analysis condition B: Retention time=1.74 min; ESI-MS(+) m/z [M+2H]²⁺: 1900.9.

### Preparation of Example 1176

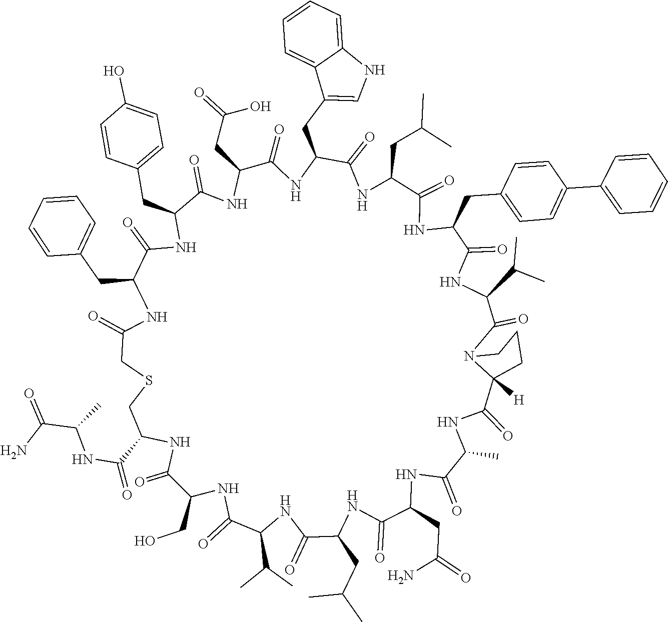

Example 1176 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, “Symphony double-coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 18-58% B over 20 minutes, then a 3-minute hold at 100% B; Flow: 20 mL/min. Fractions containing the desired product were combined and dried via centrifugal evaporation. The yield of the product was 40.4 mg, and its estimated purity by LCMS analysis was 98.7%.
Analysis condition A: Retention time=1.61 min; ESI-MS(+) m/z [M+H]⁺: 1860.1.
Analysis condition B: Retention time=1.79 min; ESI-MS(+) m/z [M+H]⁺: 1859.9.

### Preparation of Example 1177

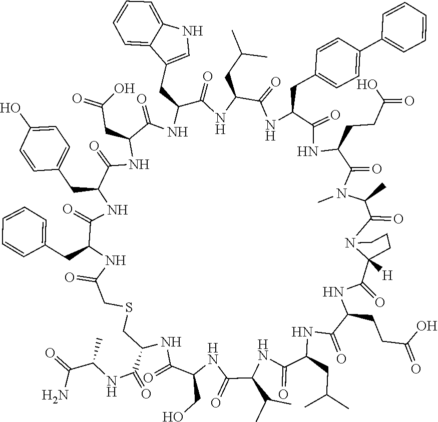

Example 1177 was prepared, using Sieber or Rink on a 50 μmol scale, following the general synthetic sequence described for the preparation of Example 1000 composed of the following general procedures: “Symphony Resin-swelling procedure”, “Symphony Single-coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 10-60% B over 20 minutes, then a 2-minute hold at 100% B; Flow: 45 mL/min. Fractions containing the desired product were combined and dried via centrifugal evaporation. The yield of the product was 8.5 mg, and its estimated purity by LCMS analysis was 99.1%.
Analysis condition A: Retention time=1.41 min; ESI-MS(+) m/z [M+2H]²⁺: 960.1.
Analysis condition B: Retention time=1.71 min; ESI-MS(+) m/z [M+H]⁺: 1919.0.

### Preparation of Example 1178

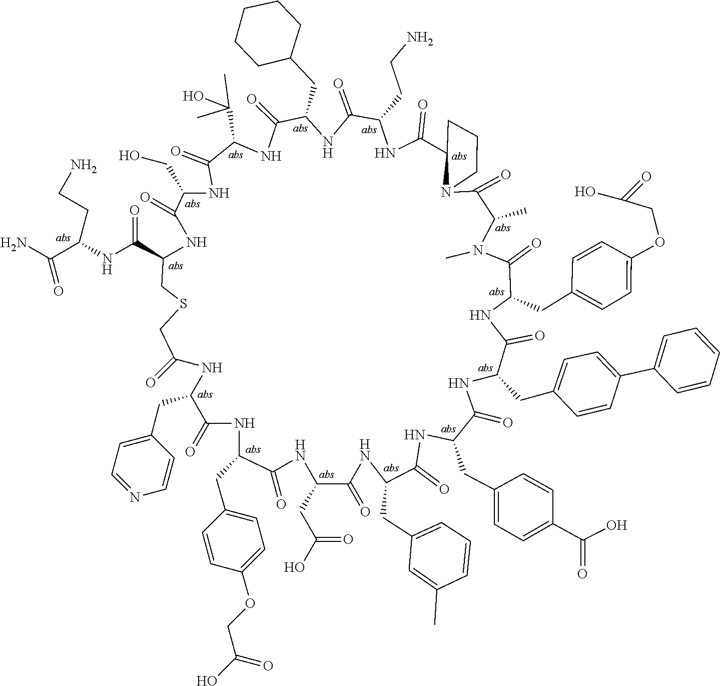 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@H]3CCCN3C(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Example 1178 was prepared, using Rink or Sieber on a 30 μmol scale, following the general synthetic sequence described for the preparation of Example 1054, composed of the following general procedures: “Prelude Resin-swelling procedure”, “Prelude Single-coupling procedure”, or “Prelude doublee-coupling procedure”, “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: a 0-minute hold at 16% B, 16-56% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 22.9 mg, and its estimated purity by LCMS analysis was 95.6%. Analysis condition A: Retention time=1.29 min; ESI-MS(+) m/z [M+2H]²⁺: 1090.3.

### Preparation of Example 1179

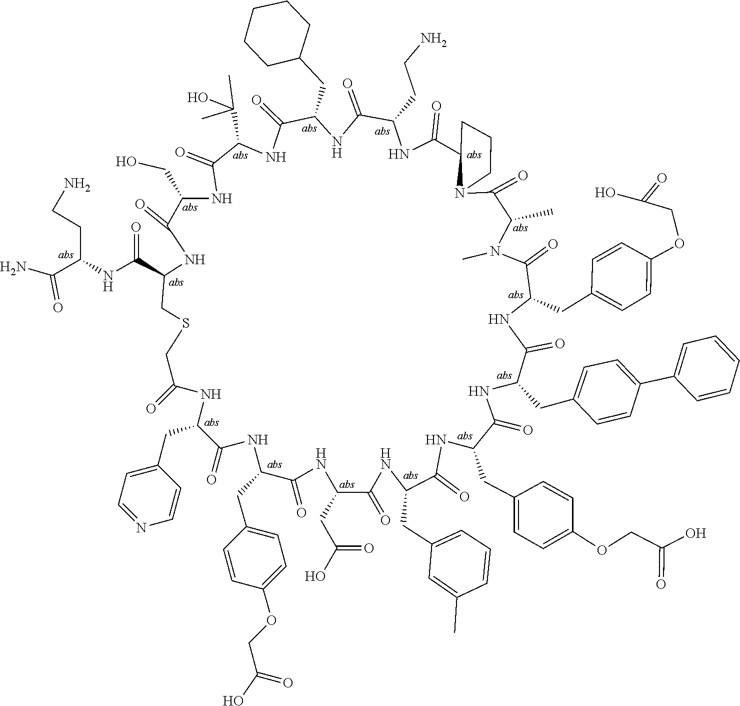 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@H]3CCCN3C(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1179 was prepared, using Rink or Sieber on a 30 μmol scale, following the general synthetic sequence described for the preparation of Example 1054, composed of the following general procedures: “Prelude Resin-swelling procedure”, “Prelude Single-coupling procedure”, or “Prelude doublee-coupling procedure”, “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-minute hold at 10% B, 10-50% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 10.6 mg, and its estimated purity by LCMS analysis was 87.4%.
Analysis condition A: Retention time=1.29 min; ESI-MS(+) m/z [M+2H]²⁺: 1105.2.
Analysis condition B: Retention time=1.53 min; ESI-MS(+) m/z [M+2H]²⁺: 1105.0.

### Preparation of Example 1180

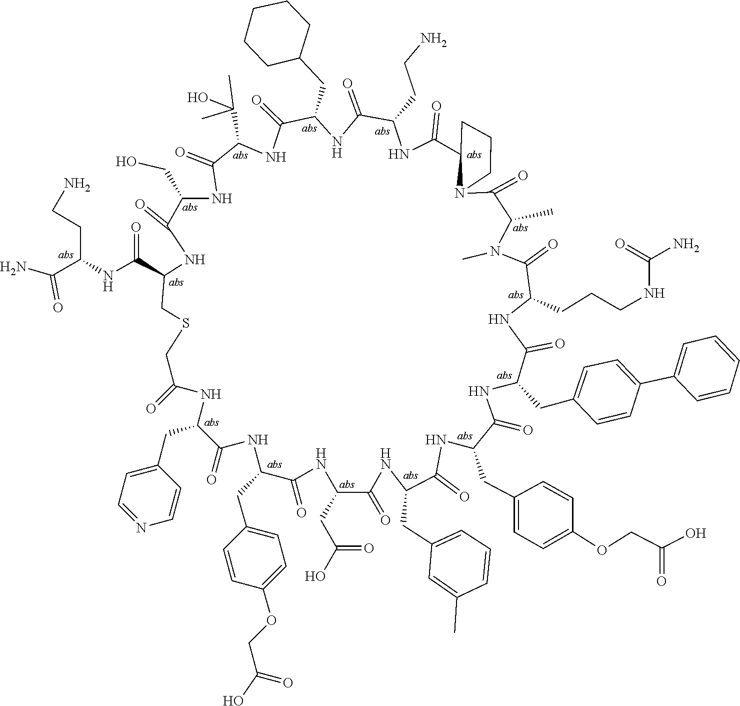 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@H]3CCCN3C(=O)[C@H](C)N(C)C(=O)[C@H](CCCNC(N)=O)NC(=O)[C@H](Cc3ccc(-c4ccccc4)cc3)NC(=O)[C@H](Cc3ccc(OCC(=O)O)cc3)NC2=O)c1]

Example 1180 was prepared, using Rink or Sieber on a 30 μmol scale, following the general synthetic sequence described for the preparation of Example 1054, composed of the following general procedures: “Prelude Resin-swelling procedure”, “Prelude Single-coupling procedure”, or “Prelude doublee-coupling procedure”, “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”. “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: a 0-minute hold at 14% B, 14-54% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 7.2 mg, and its estimated purity by LCMS analysis was 95.6%.
Analysis condition A: Retention time=1.3 min; ESI-MS(+) m/z [M+2H]²⁺: 1072.9.
Analysis condition B: Retention time=1.52 min; ESI-MS(+) m/z [M+2H]²⁺: 1073.3.

### Preparation of Example 1181

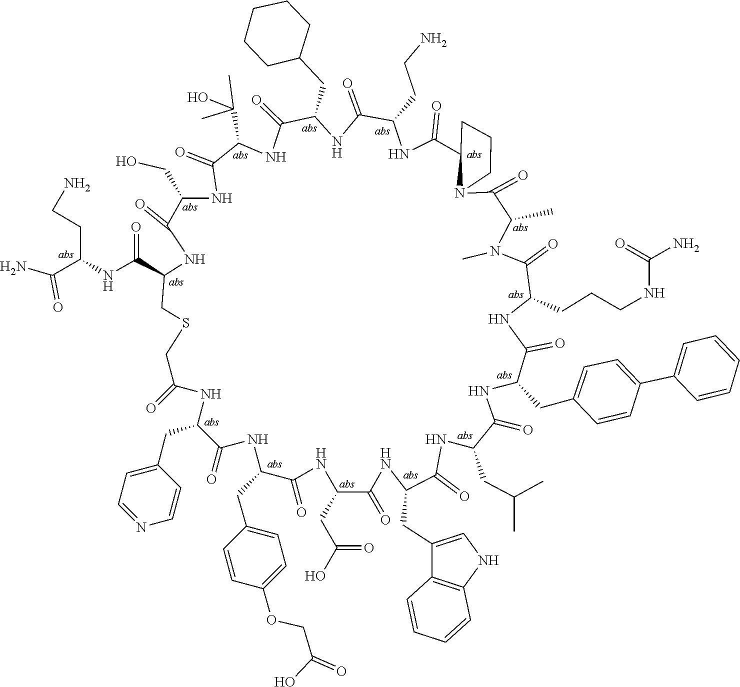 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CCN)C(N)=O)NC(=O)[C@H](CO)NC(=O)[C@H](C(C)(C)O)NC(=O)[C@H](CC2CCCCC2)NC(=O)[C@H](CCN)NC(=O)[C@H]2CCCN2C(=O)[C@H](C)N(C)C(=O)[C@H](CCCNC(N)=O)NC(=O)[C@H](Cc2ccc(-c3ccccc3)cc2)NC1=O]

Example 1181 was prepared, using Rink or Sieber on a 30 μmol scale, following the general synthetic sequence described for the preparation of Example 1054, composed of the following general procedures: “Prelude Resin-swelling procedure”, “Prelude Single-coupling procedure”, or “Prelude doublee-coupling procedure”, “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”. “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: a 0-minute hold at 16% B, 16-56% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 8.7 mg, and its estimated purity by LCMS analysis was 95.2%.
Analysis condition A: Retention time=1.48 min; ESI-MS(+) m/z [M+2H]²⁺: 1032.1.
Analysis condition B: Retention time=1.50 min; ESI-MS(+) m/z [M+2H]²⁺: 1032.1.

### Preparation of Example 1182

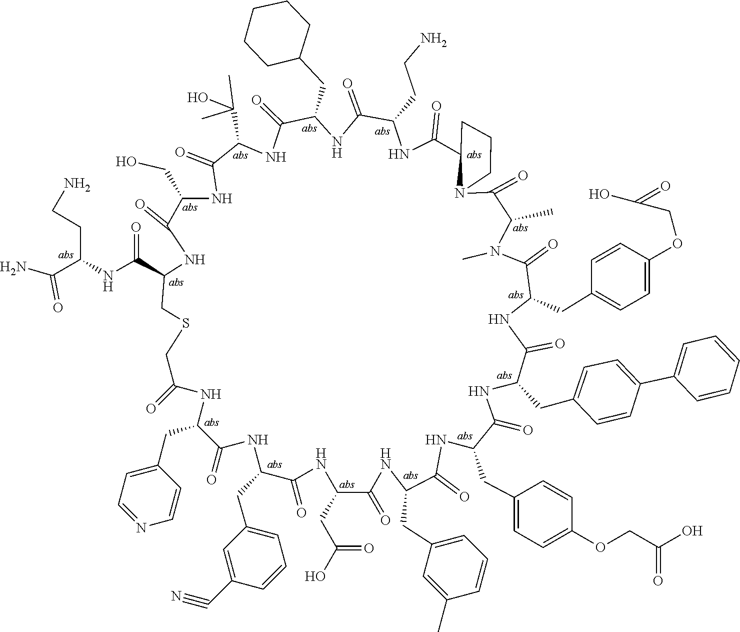 [SMILES: Cc1cccc(C[C@@H]2NC(=O)[C@H](CC(=O)O)NC(=O)[C@H](Cc3cccc(C#N)c3)NC(=O)[C@H](Cc3ccncc3)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@H]3CCCN3C(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1182 was prepared, using Rink or Sieber on a 30 μmol scale, following the general synthetic sequence described for the preparation of Example 1054, composed of the following general procedures: “Prelude Resin-swelling procedure”, “Prelude Single-coupling procedure or “Prelude doublee-coupling procedure”, “Symphony X Resin-swelling procedure”, “Symphony X Single-coupling procedure”, “Symphony X Single-Coupling Manual Addition Procedure A” was followed with Fmoc-Phe(3-CN)—OH; “Symphony X Chloroacetic Anhydride coupling procedure”, “Symphony X Final rinse and dry procedure”, “Global Deprotection Method A”. “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: a 0-minute hold at 18% B, 18-58% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 23.7 mg, and its estimated purity by LCMS analysis was 89%.
Analysis condition A: Retention time=1.52 min; ESI-MS(+) m/z [M+H]²⁺: 1081.2.
Analysis condition B: Retention time=1.58 min; ESI-MS(+) m/z [M+H]²⁺: 1081.1.

### Preparation of Example 1183

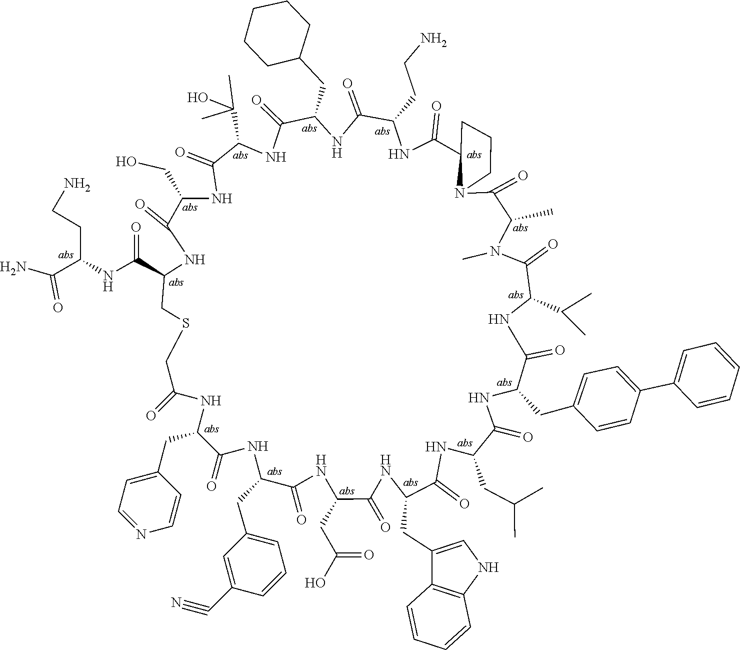 [SMILES: CC(C)C[C@@H]1NC(=O)[C@H](Cc2c[nH]c3ccccc23)NC(=O)[C@H](CC(=O)O)NC(=O)[C@H](Cc2cccc(C#N)c2)NC(=O)[C@H](Cc2ccncc2)NC(=O)CSC[C@@H](C(=O)N[C@@H](CCN)C(N)=O)NC(=O)[C@H](CO)NC(=O)[C@H](C(C)(C)O)NC(=O)[C@H](CC2CCCCC2)NC(=O)[C@H](CCN)NC(=O)[C@H]2CCCN2C(=O)[C@H](C)N(C)C(=O)[C@H](C(C)C)NC(=O)[C@H](Cc2ccc(-c3ccccc3)cc2)NC1=O]

Example 1183 was prepared, using Rink or Sieber on a 30 μmol scale, following the general synthetic sequence described for the preparation of Example 1054, composed of the following general procedures: “Prelude Resin-swelling procedure”, “Prelude Single-coupling procedure or “Prelude doublee-coupling procedure”, “Symphony X Resin-swelling procedure”, “Symphony X Single-coupling procedure “Symphony X Single-Coupling Manual Addition Procedure A” was followed with Fmoc-NMe-Orn(Boc)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: a 0-minute hold at 18% B, 18-58% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 14.1 mg, and its estimated purity by LCMS analysis was 92.9%.
Analysis condition A: Retention time=1.76 min; ESI-MS(+) m/z [M+H]⁺: 1955.1.
Analysis condition B: Retention time=1.66 min; ESI-MS(+) m/z [M+2H]²⁺: 978.0.

### Preparation of Example 1184

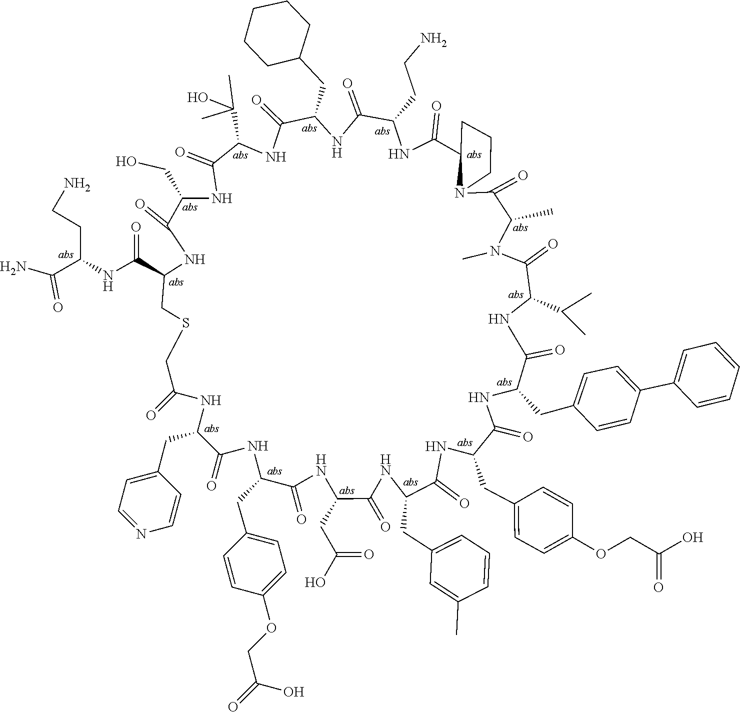 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@H]3CCCN3C(=O)[C@H](C)N(C)C(=O)[C@H](C(C)C)NC(=O)[C@H](Cc3ccc(-c4ccccc4)cc3)NC(=O)[C@H](Cc3ccc(OCC(=O)O)cc3)NC2=O)c1]

Example 1184 was prepared, using Sieber or Rink resin on a 30 μmol scale, following the general synthetic sequence described for the preparation of Example 1054, composed of the following general procedures: “Prelude Resin-swelling procedure”, “Prelude Single-coupling procedure”, or “Prelude doublee-coupling procedure”, “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: a 0-minute hold at 16% B, 16-56% B over 24 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 13.4 mg, and its estimated purity by LCMS analysis was 85.4%.
Analysis condition A: Retention time=1.34 min; ESI-MS(+) m/z [M+2H]²⁺: 1044.1.
Analysis condition B: Retention time=1.51 min; ESI-MS(+) m/z [M+2H]²⁺: 1044.0.

### Preparation of Example 1185

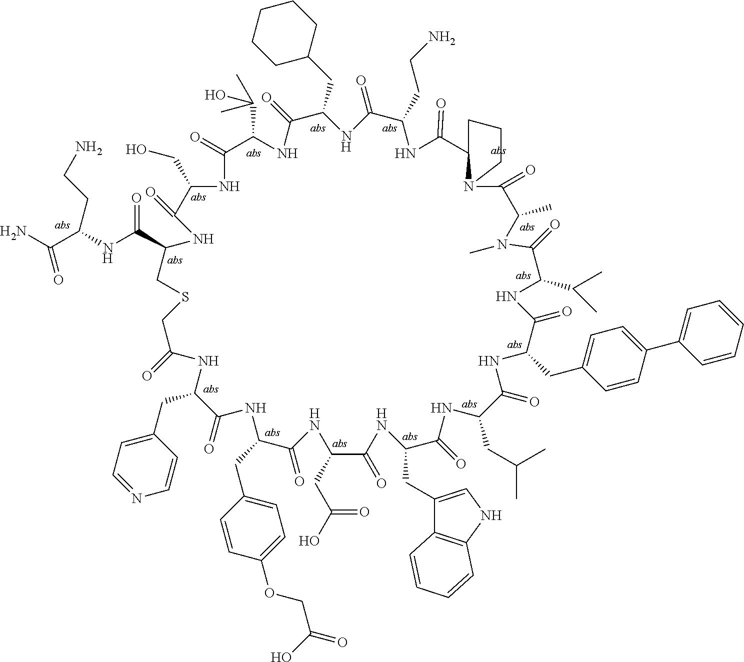

Example 1185 was prepared, using Sieber or Rink resin on a 30 μmol scale, following the general synthetic sequence described for the preparation of Example 1054, composed of the following general procedures: “Prelude Resin-swelling procedure”, “Prelude Single-coupling procedure”, or “Prelude doublee-coupling procedure”, “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: a 0-minute hold at 16% B, 16-56% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 9.7 mg, and its estimated purity by LCMS analysis was 96%.
Analysis condition A: Retention time=1.48 min; ESI-MS(+) m/z [M+2H]²⁺: 1003.2.
Analysis condition B: Retention time=1.51 min; ESI-MS(+) m/z [M+2H]²⁺: 1003.0.

### Preparation of Example 1186

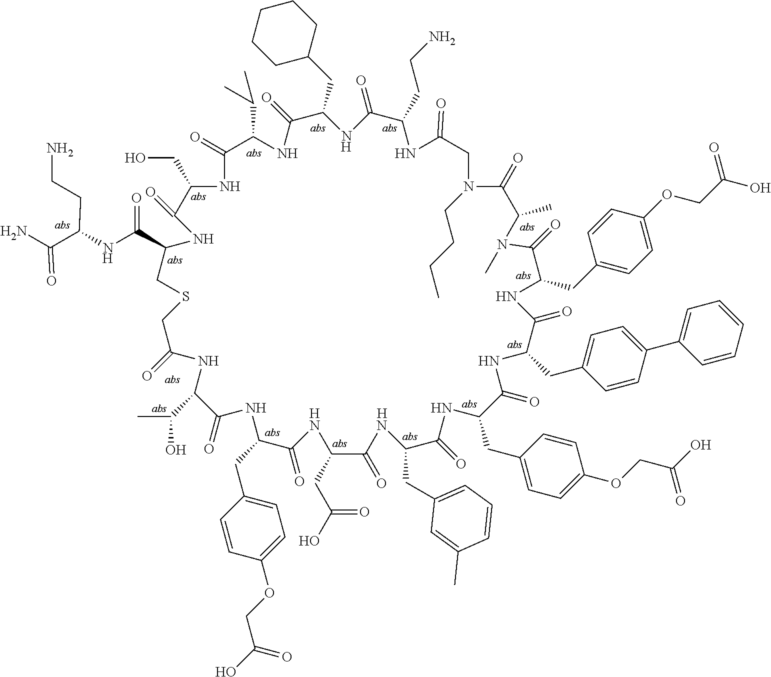

Example 1186 was prepared, using Sieber or Rink on a 25 μmol scale, following the general synthetic sequence described for the preparation of Example 1002, composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-N(nBu)-Gly-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile: water with 10-mM ammonium acetate; Mobile Phase B: 95:5 acetonitrile: water with 10-mM ammonium acetate; Gradient: a 0-minute hold at 8% B, 8-48% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 6.6 mg, and its estimated purity by LCMS analysis was 90.9%.
Analysis condition A: Retention time=1.32 min; ESI-MS(+) m/z [M+2H]²⁺: 1082.1.
Analysis condition B: Retention time=1.69 min; ESI-MS(+) m/z [M+2H]²⁺: 1082.1.

### Preparation of Example 1187

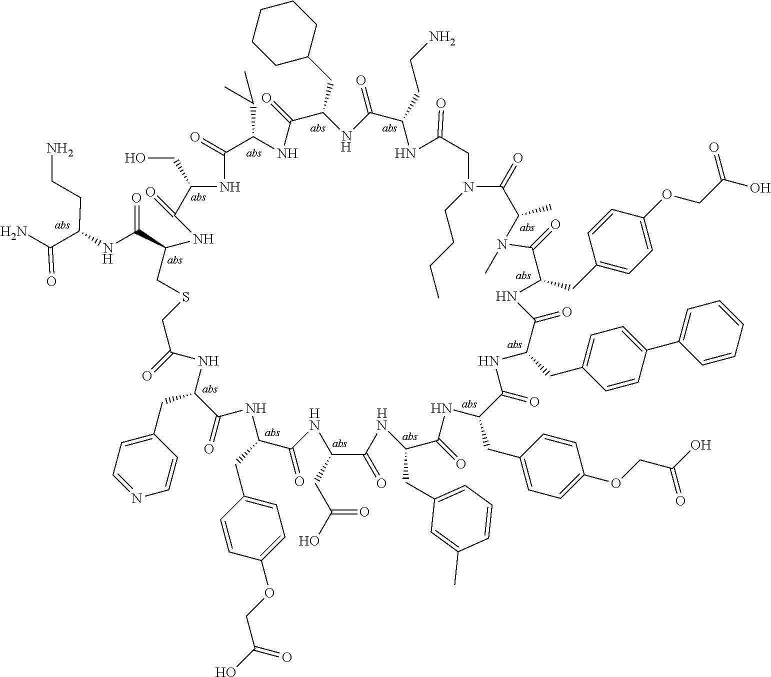

Example 1187 was prepared, using Sieber or Rink on a 25 μmol scale, following the general synthetic sequence described for the preparation of Example 1002, composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-N(nBu)-Gly-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile: water with 10-mM ammonium acetate; Mobile Phase B: 95:5 acetonitrile: water with 10-mM ammonium acetate; Gradient: a 0-minute hold at 9% B, 9-49% B over 20 minutes, then a 0-minute hold at 100% B; Flow Rate: 50 mL/min; Column Temperature: 25 C. Fraction collection was triggered by MS and UV signals. Fractions containing the desired product were combined and dried via centrifugal evaporation. The yield of the product was 1.2 mg, and its estimated purity by LCMS analysis was 93.6%.
Analysis condition A: Retention time=1.34 min; ESI-MS(+) m/z [M+2H]²: 1105.2.
Analysis condition B: Retention time=1.64 min; ESI-MS(+) m/z [M+2H]²⁺: 1105.2.

### Preparation of Example 1188

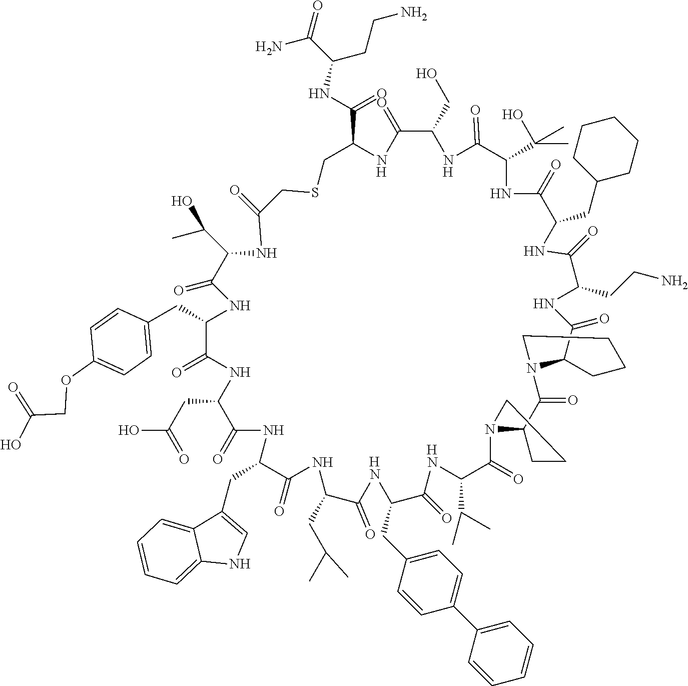

Example 1188 was prepared, using Rink or Sieber Resin on a 50 μmol scale, following the general synthetic sequence described for the preparation of Example 1002, composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-D-Pip-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-minute hold at 21% B, 21-61% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 21.1 mg, and its estimated purity by LCMS analysis was 91%. Analysis condition 1: Retention time=1.71 min; ESI-MS(+) m/z [M+2H]²⁺: 1984.

### Preparation of Example 1189

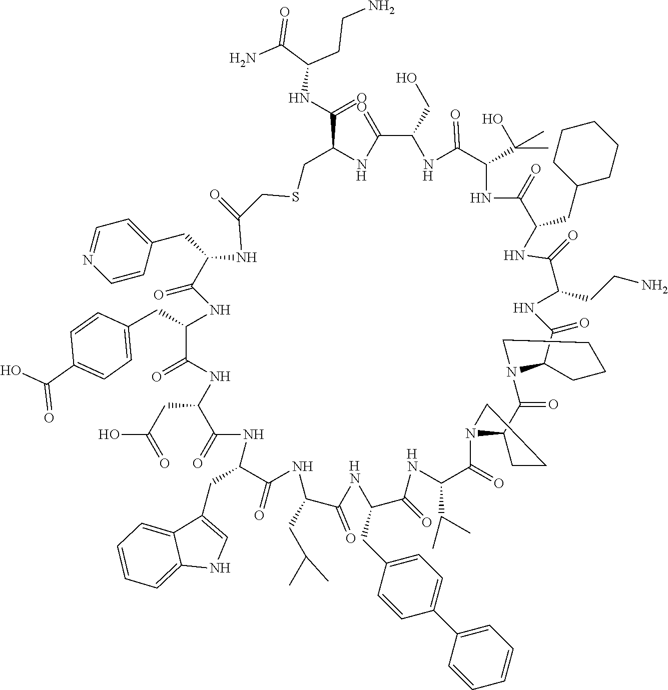

Example 1189 was prepared, using Rink or Sieber Resin on a 50 μmol scale, following the general synthetic sequence described for the preparation of Example 1002, composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-D-Pip-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-minute hold at 17% B, 17-57% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 27.6 mg, and its estimated purity by LCMS analysis was 98.4%. Analysis condition 1: Retention time=1.61 min; ESI-MS(+) m/z [M+2H]²⁺: 1001.1.

### Preparation of Example 1190

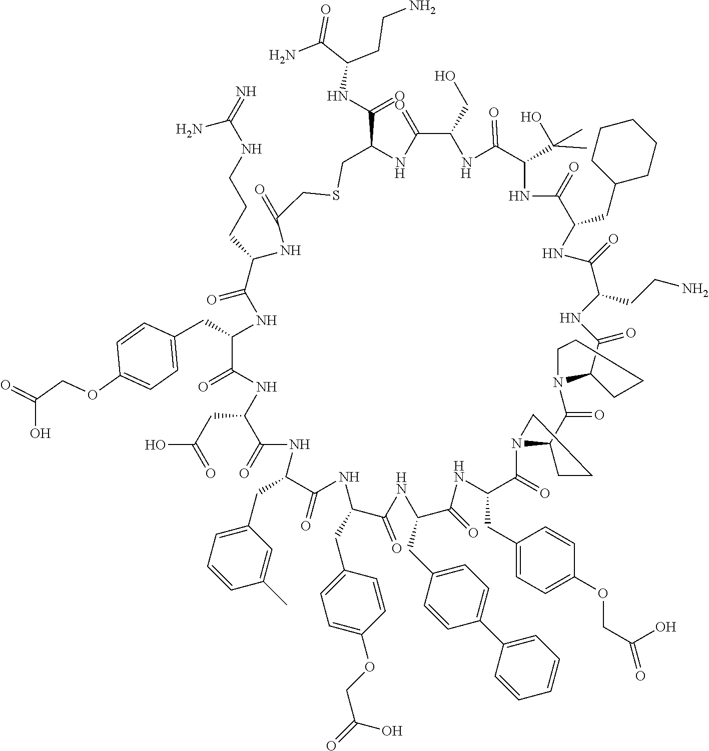

Example 1190 was prepared, using Rink or Sieber Resin on a 50 μmol scale, following the general synthetic sequence described for the preparation of Example 1002, composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-D-Pip-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-minute hold at 14% B, 14-54% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at 20% B, 20-60% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 15.9 mg, and its estimated purity by LCMS analysis was 96.8%. Analysis condition 1: Retention time=1.68 min; ESI-MS(+) m/z [M+2H]²⁺: 1122.3.

### Preparation of Example 1191

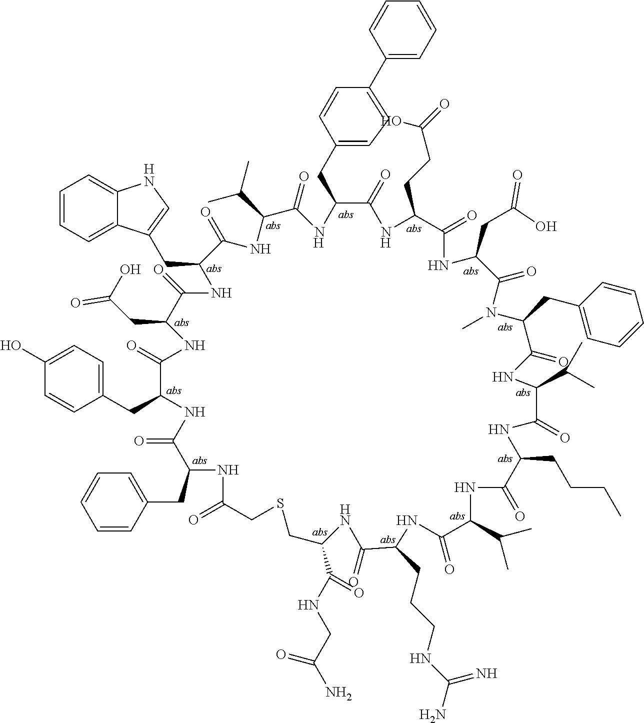

To a 45-mL polypropylene solid-phase reaction vessel was added Sieber resin or Rink resin (70 mg for Sieber or 100 mg for Rink, 0.050 mmol), and the reaction vessel was placed on the Symphony X peptide synthesizer. The following procedures were then performed sequentially:

“Symphony X Resin-swelling procedure” was followed; “Symphony X Single-coupling procedure” was followed with Fmoc-Gly-O H; “Symphony X Single-coupling procedure” was followed with Fmoc-Cys(Trt)-OH; “Symphony X Single-coupling procedure” was followed with Fmoc-Arg(Pbf)-OH; “Symphony X Single-coupling procedure” was followed with Fmoc-Val-OH; “Symphony X Single-coupling procedure” was followed with Fmoc-Nle-OH; “Symphony X Single-coupling procedure” was followed with Fmoc-Val-OH; “Symphony X Single-coupling procedure” was followed with Fmoc-N-Me-Phe-OH; “Symphony X Single-coupling procedure” or “Symphony X double-coupling procedure” was followed with Fmoc-Asp(tBu)-OH; “Symphony X Single-coupling procedure” was followed with Fmoc-Glu(tBu)-OH; “Symphony X Single-coupling procedure” was followed with Fmoc-Bip-OH; “Symphony X Single-coupling procedure” was followed with Fmoc-Val-OH; “Symphony X Single-coupling procedure” was followed with Fmoc-Trp(Boc)-OH; “Symphony X Single-coupling procedure” was followed with Fmoc-Asp(tBu)-OH; “Symphony X Single-coupling procedure” was followed with Tyr(tBu)-OH; “Symphony X Single-coupling procedure” was followed with Fmoc-Phe-OH; “Symphony X Chloroacetic Anhydride coupling procedure” was followed; “Symphony X Final rinse and dry procedure” was followed; “Global Deprotection Method A” was followed; “Cyclization Method A” was followed.

The crude material was purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 10-60% B over 20 minutes, then a 2-minute hold at 100% B; Flow: 45 mL/min. Fractions containing the desired product were combined and dried via centrifugal evaporation. The yield of the product was 38.3 mg, and its estimated purity by LCMS analysis was 96.2%.
Analysis condition A: Retention time=1.43 min; ESI-MS(+) m/z [M+H]⁺: 1013.1.
Analysis condition B: Retention time=1.68 min; ESI-MS(+) m/z [M+H]⁺: 1013.2.

### Preparation of Example 1192

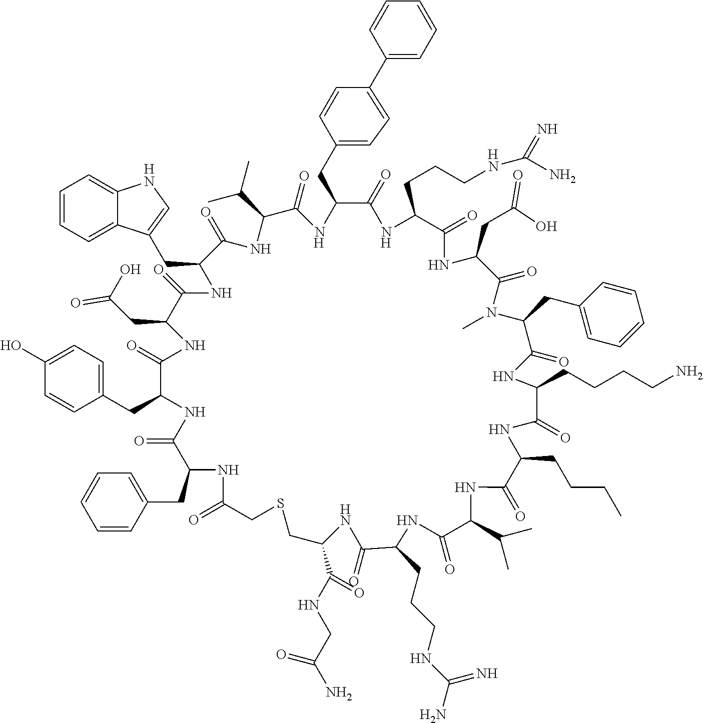

Example 1192 was prepared, using Rink or Sieber on a 50 μmol scale, following the general synthetic sequence described for the preparation of Example 1191, composed of the following general procedures: “Symphony X Resin-swelling procedure”, “Symphony X Single-coupling procedure”, or “Symphony X double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 18-58% B over 27 minutes, then a 3-minute hold at 100% B; Flow: 20 mL/min. Fractions containing the desired product were combined and dried via centrifugal evaporation. The material was further purified via preparative LC/MS with the following conditions: Column: XBridge C18, 30×150 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 5-55% B over 20 minutes, then a 2-minute hold at 100% B; Flow: 40 mL/min. Fractions containing the desired product were combined and dried via centrifugal evaporation. The yield of the product was 9.8 mg, and its estimated purity by LCMS analysis was 98.8%.
Analysis condition A: Retention time=1.49 min; ESI-MS(+) m/z [M+3H]³⁺: 694.5.
Analysis condition B: Retention time=1.46 min; ESI-MS(+) m/z [M+3H]³⁺: 694.3.

### Preparation of Example 1193

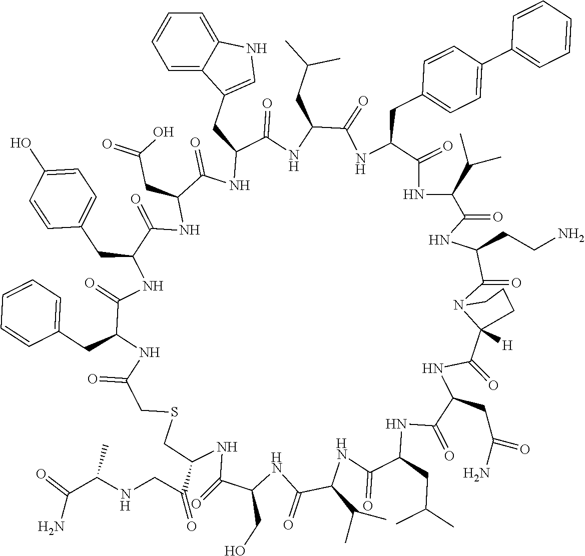

Example 1193 was prepared, using Rink or Sieber on a 50 μmol scale, following the general synthetic sequence described for the preparation of Example 1191, composed of the following general procedures: “Symphony X Resin-swelling procedure”, “Symphony X Single-coupling procedure”, or “Symphony X double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 15-55% B over 20 minutes, then a 3-minute hold at 100% a B; Flow: 20 mL/min. Fractions containing the desired product were combined and dried via centrifugal evaporation. The yield of the product was 16.5 mg, and its estimated purity by LCMS analysis was 100/L.
Analysis condition A: Retention time=1.86 mm; ESI-MS(+) m/z [M+H]⁺: 1889.1.
Analysis condition B: Retention time=1.92 min; ESI-MS(+) m/z [M+H]⁺: 1889.3.

### Preparation of Example 1194

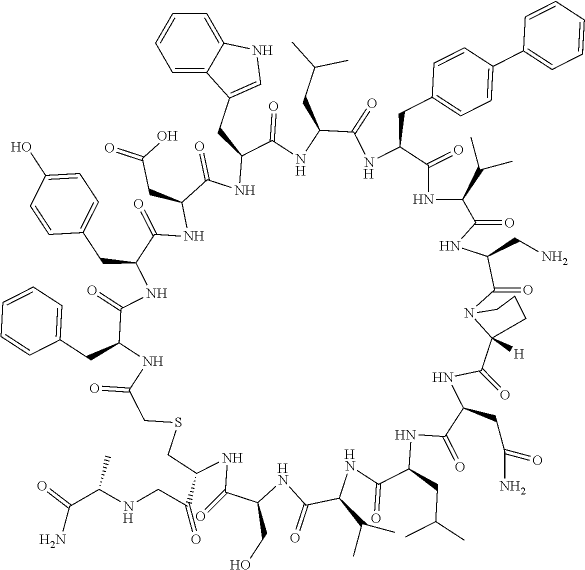

Example 1194 was prepared, using Rink or Sieber on a 50 μmol scale, following the general synthetic sequence described for the preparation of Example 1191, composed of the following general procedures: “Symphony X Resin-swelling procedure”, “Symphony X Single-coupling procedure”, or “Symphony X double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 20-60% B over 20 minutes, then a 5-minute hold at 100% B; Flow: 20 mL/min. Fractions containing the desired product were combined and dried via centrifugal evaporation. The yield of the product was 29.2 mg, and its estimated purity by LCMS analysis was 99.2%.
Analysis condition A: Retention time=1.96 min; ESI-MS(+) m/z [M+2H]²⁺: 938.1.
Analysis condition B: Retention time=1.88 min; ESI-MS(+) m/z [M+H]⁺: 1875.3.

### Preparation of Example 1195

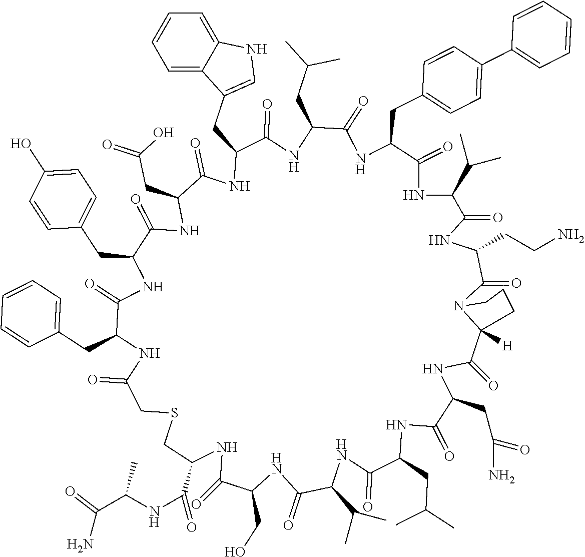

Example 1195 was prepared, using Rink or Sieber on a 50 μmol scale, following the general synthetic sequence described for the preparation of Example 1191, composed of the following general procedures: “Symphony X Resin-swelling procedure”, “Symphony X Single-coupling procedure”, or “Symphony X double-coupling procedure”, “Symphony X single shot procedure” was followed with Fmoc-D-Dab(Boc)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 20 minutes, then a 3-minute hold at 100% B; Flow: 20 mL/min. Fractions containing the desired product were combined and dried via centrifugal evaporation. The yield of the product was 44.7 mg, and its estimated purity by LCMS analysis was 99%.
Analysis condition A: Retention time=1.76 min; ESI-MS(+) m/z [M+H]⁺: 1888.9.

### Preparation of Example 1196

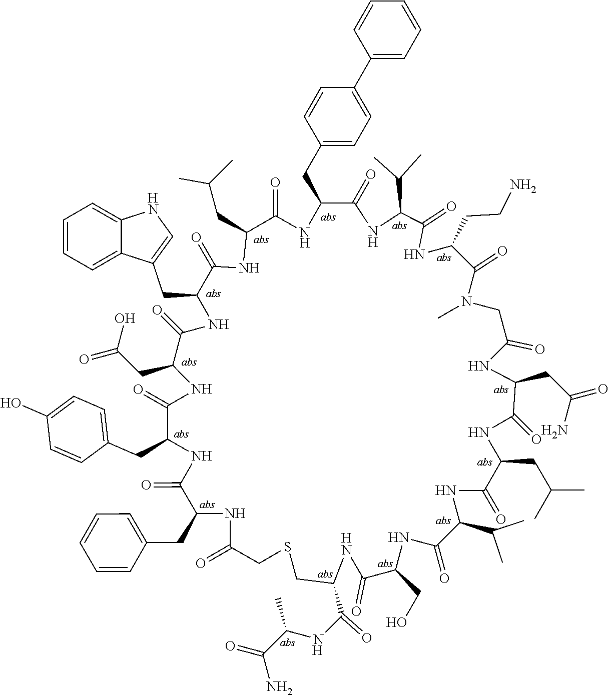

Example 1196 was prepared, using Rink or Sieber on a 50 μmol scale, following the general synthetic sequence described for the preparation of Example 1191, composed of the following general procedures: “Symphony X Resin-swelling procedure”, “Symphony X Single-coupling procedure”, or “Symphony X double-coupling procedure”, “Symphony X single shot procedure” was followed with Fmoc-D-Dab(Boc)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 20 minutes, then a 3-minute hold at 100% B; Flow: 20 mL/min. Fractions containing the desired product were combined and dried via centrifugal evaporation. The material was further purified via preparative LC/MS with the following conditions: Column: XBridge C18, 19×200 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 32-57% B over 25 minutes, then a 2-minute hold at 57% B; Flow: 20 mL/min. Fractions containing the desired product were combined and dried via centrifugal evaporation. The yield of the product was 47.4 mg, and its estimated purity by LCMS analysis was 96.5%.
Analysis condition A: Retention time=1.72 min; ESI-MS(+) m/z [M+H]⁺: 1863.9.
Analysis condition B: Retention time=1.75 min; ESI-MS(+) m/z [M+H]⁺: 1863.9.

### Preparation of Example 1197

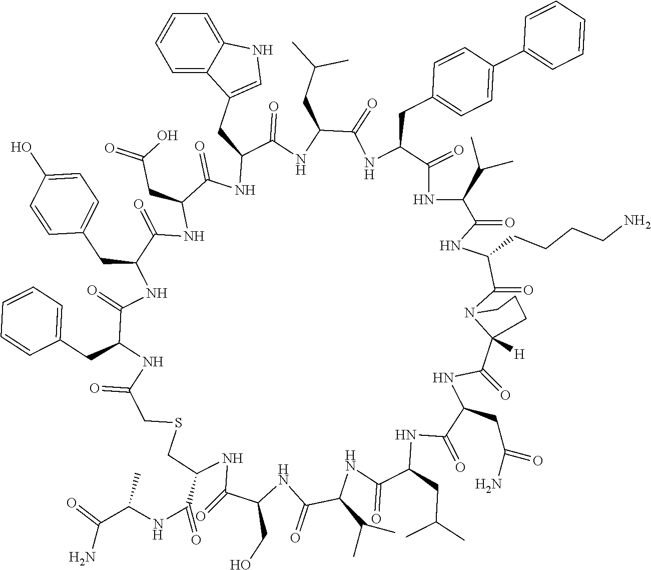

To a 45-mL polypropylene solid-phase reaction vessel was added Sieber resin or Rink resin (210 mg for Sieber or 300 mg for Rink, 0.300 mmol), and the reaction vessel was placed on the Prelude peptide synthesizer. The following procedures were then performed sequentially:

“Prelude Resin-swelling procedure” was followed; “Prelude Single-coupling procedure” was followed with Fmoc-Ala-OH; “Prelude Single-coupling procedure” was followed with Fmoc-Cys(Trt)-OH; “Prelude Single-coupling procedure” was followed with Fmoc-Ser(tBu)-OH; “Prelude Single-coupling procedure” was followed with Fmoc-Val-OH; “Prelude Single-coupling procedure” was followed with Fmoc-Leu-OH; “Prelude Single-coupling procedure” was followed with Fmoc-Asn(Trt)-OH; “Prelude Single-coupling procedure” was followed with Fmoc-D-Pro-OH; “Prelude Single-coupling procedure” was followed with Fmoc-D-Lys(Boc)-OH; “Prelude Single-coupling procedure” was followed with Fmoc-Val-OH;

The resin was split into 0.050 mmol and was transferred to a different 45-mL polypropylene solid-phase reaction vessel, and the reaction vessel was placed on the Symphony peptide synthesizer. The following procedures were then performed sequentially:

“Symphony Single-coupling procedure” was followed with Fmoc-Bip-OH; “Symphony Single-coupling procedure” was followed with Fmoc-Leu-OH; “Symphony Single-coupling procedure” was followed with Fmoc-Trp(Boc)-OH; “Symphony Single-coupling procedure” was followed with Fmoc-Asp(tBu)-OH; “Symphony Single-coupling procedure” was followed with Tyr(tBu)-OH; “Symphony Single-coupling procedure” was followed with Fmoc-Phe-OH; “Symphony Chloroacetic Anhydride coupling procedure” was followed; “Global Deprotection Method A” was followed; “Cyclization Method A” was followed.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-55% B over 20 minutes, then a 4-minute hold at 100% B; Flow: 20 mL/min. Fractions containing the desired product were combined and dried via centrifugal evaporation. The yield of the product was 17.7 mg, and its estimated purity by LCMS analysis was 98.9%.
Analysis condition B: Retention time=1.77 min; ESI-MS(+) m/z [M+H]⁺: 1916.9.

### Preparation of Example 1198

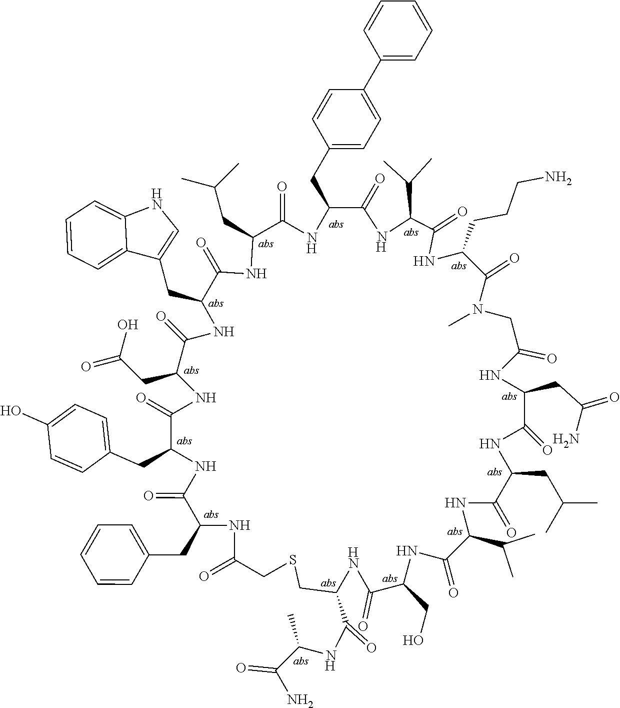

Example 1198 was prepared, using Rink or Sieber on a 50 μmol scale, following the general synthetic sequence described for the preparation of Example 1197, composed of the following general procedures: “Prelude Resin-swelling procedure”, “Prelude Single-coupling procedure”, “Symphony Resin-swelling procedure”, “Symphony Single-coupling procedure”. “Symphony Chloroacetic Anhydride coupling procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-55% B over 20 minutes, then a 5-minute hold at 100% B; Flow: 20 mL/min. Fractions containing the desired product were combined and dried via centrifugal evaporation. The yield of the product was 28.7 mg, and its estimated purity by LCMS analysis was 97.4%.
Analysis condition A: Retention time=1.81 min; ESI-MS(+) m/z [M+H]⁺: 1878.0.
Analysis condition B: Retention time=1.76 min; ESI-MS(+) m/z [M+H]⁺: 1877.0.

### Preparation of Example 1199

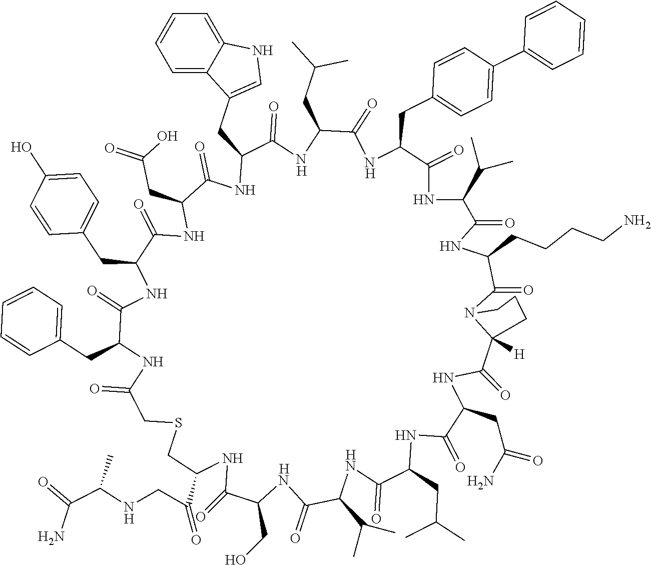

Example 1199 was prepared, using Rink or Sieber on a 50 μmol scale, following the general synthetic sequence described for the preparation of Example 1191, composed of the following general procedures: “Symphony X Resin-swelling procedure”, “Symphony X Single-coupling procedure”, or “Symphony X double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 20-60% B over 20 minutes, then a 5-minute hold at 100% B; Flow: 20 mL/min. Fractions containing the desired product were combined and dried via centrifugal evaporation. The yield of the product was 22.1 mg, and its estimated purity by LCMS analysis was 100%.
Analysis condition A: Retention time=1.85 min; ESI-MS(+) m/z [M+H]⁺: 1918.3.
Analysis condition B: Retention time=1.85 min; ESI-MS(+) m/z [M+2H]²⁺: 959.8.

### Preparation of Example 1200

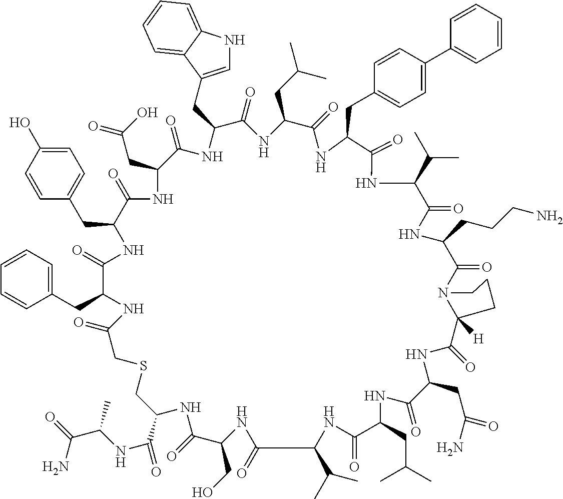

Example 1200 was prepared, using Rink or Sieber on a 50 μmol scale, following the general synthetic sequence described for the preparation of Example 1191, composed of the following general procedures: “Symphony X Resin-swelling procedure”, “Symphony X Single-coupling procedure”, or “Symphony X double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 18-58% B over 20 minutes, then a 3-minute hold at 100% B; Flow: 20 mL/min. Fractions containing the desired product were combined and dried via centrifugal evaporation. The yield of the product was 35.4 mg, and its estimated purity by LCMS analysis was 100%.
Analysis condition A: Retention time=1.85 min; ESI-MS(+) m/z [M+2H]²⁺: 952.2.
Analysis condition B: Retention time=1.9 min; ESI-MS(+) m/z [M+H]⁺: 1904.0.

Examples 1201-1455 were prepared according to those described in the general instrumental procedures and those described for Examples 1000, 1002, 1054, 1191 and 1197.

### Preparation of Example 1201

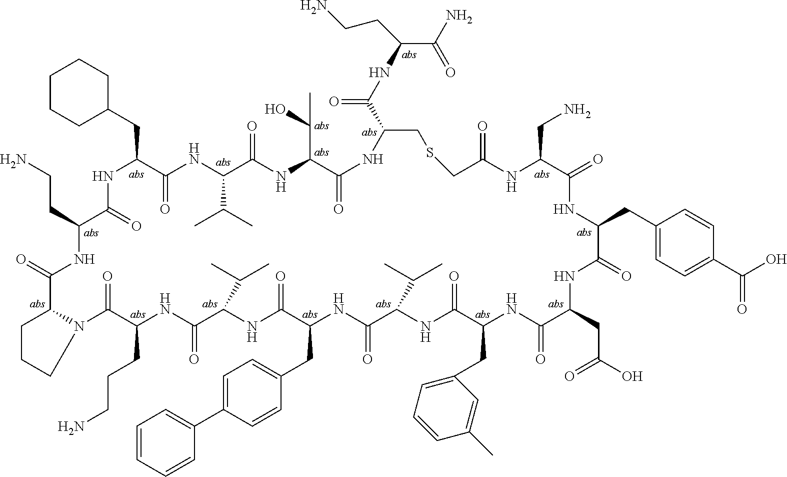 [SMILES: Cc1cccc(C[C@@H]2NC(=O)[C@H](CC(=O)O)NC(=O)[C@H](Cc3ccc(C(=O)O)cc3)NC(=O)[C@H](CN)NC(=O)CSC[C@@H](C(=O)N[C@@H](CCN)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@H]3CCCN3C(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Example 1456 was prepared on a 50 μmol scale. The yield of the product was 8 mg, and its estimated purity by LCMS analysis was 95.6%. Analysis condition B: Retention time=1.3 min; ESI-MS(+) m,% z [M+3H]³⁺: 634.3.

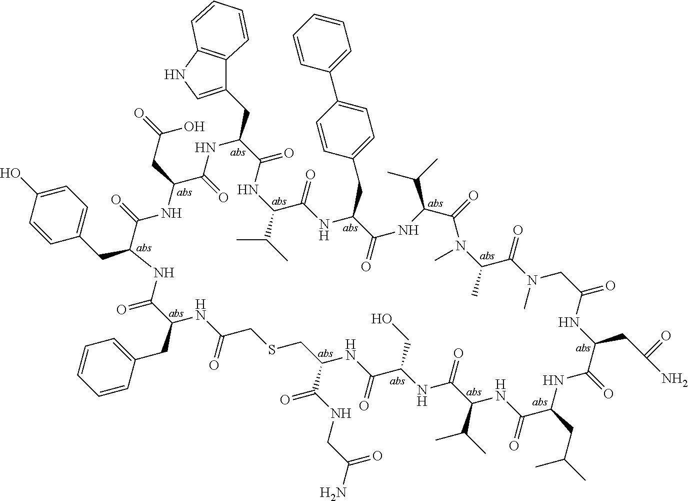 [SMILES: CC(C)C[C@@H]1NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC1=O]

### Preparation of Example 1202

Example 1202 was prepared on a 50 μmol scale. The yield of the product was 24.2 mg, and its estimated purity by LCMS analysis was 99%. Analysis condition A: Retention time=1.45 min; ESI-MS(+) m/z [M+H]⁺: 1819.8.

### Preparation of Example 1203

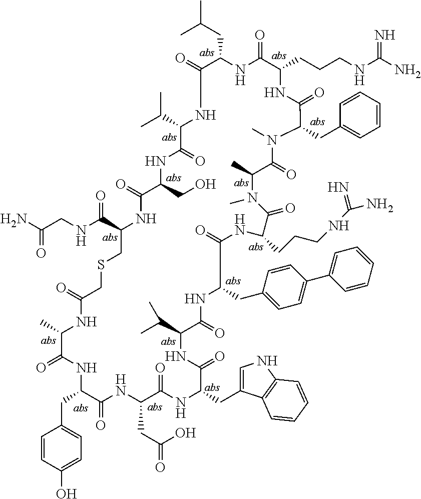 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](C)NC(=O)CSC[C@@H](C(=O)NCC(N)=O)NC(=O)[C@H](CO)NC(=O)[C@H](C(C)C)NC1=O]

Example 1203 was prepared on a 50 gmol scale. The yield of the product was 24.5 mg, and its estimated purity by LCMS analysis was 100%. Analysis condition A: Retention time=1.92 min; ESI-MS(+) m/z [M+H]⁺: 1932.9.

### Preparation of Example 1204

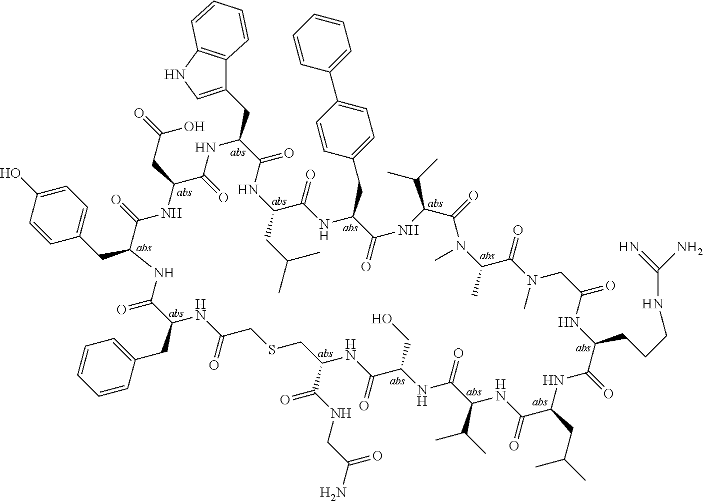 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CCCNC(=N)N)NC(=O)CN(C)C(=O)[C@H](C)N(C)C(=O)[C@H](C(C)C)NC(=O)[C@H](Cc2ccc(-c3ccccc3)cc2)NC1=O]

Example 1204 was prepared on a 50 μmol scale. The yield of the product was 17.9 mg, and its estimated purity by LCMS analysis was 93.2%. Analysis condition A: Retention time=1.58 min; ESI-MS(+) m/z [M+H]⁺: 1877.

### Preparation of Example 1205

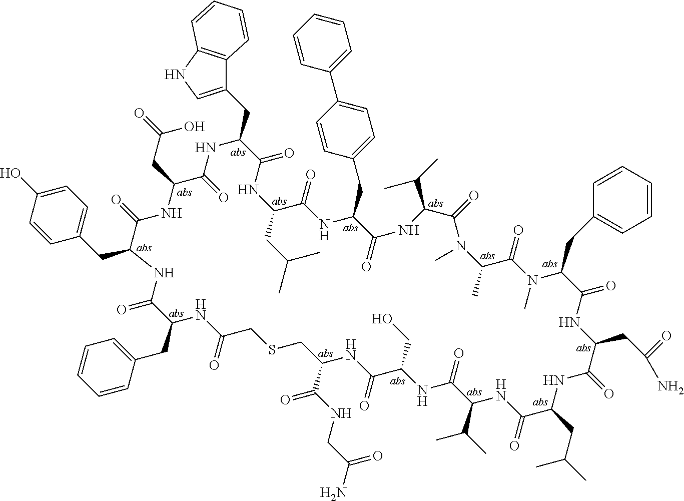 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CC(N)=O)NC(=O)[C@H](Cc2ccccc2)N(C)C(=O)[C@H](C)N(C)C(=O)[C@H](C(C)C)NC(=O)[C@H](Cc2ccc(-c3ccccc3)cc2)NC1=O]

Example 1205 was prepared on a 50 μmol scale. The yield of the product was 17.9 mg, and its estimated purity by LCMS analysis was 92.4%. Analysis condition A: Retention time=1.78 min: ESI-MS(+) m/z [M+H]⁺: 1924.1.

### Preparation of Example 1206

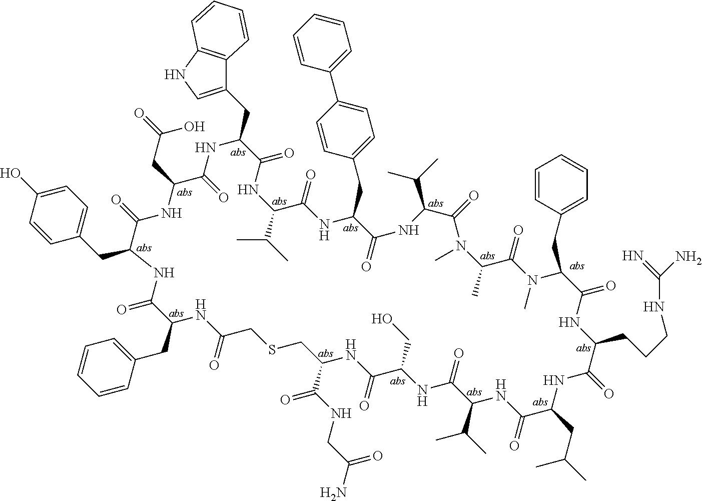 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC1=O]

Example 1206 was prepared on a 50 μmol scale. The yield of the product was 24.8 mg, and its estimated purity by LCMS analysis was 96.3%. Analysis condition A: Retention time=1.83 min; ESI-MS(+) m/z [M+H]⁺: 1952.

### Preparation of Example 1207

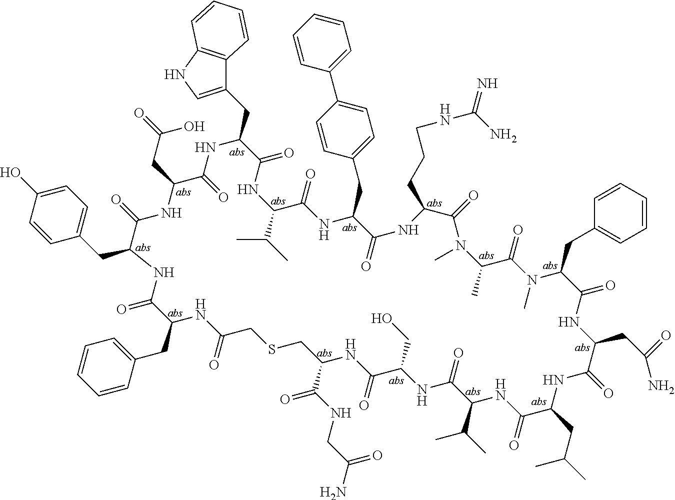 [SMILES: CC(C)C[C@@H]1NC(=O)[C@H](CC(N)=O)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC1=O]

Example 1207 was prepared on a 50 μmol scale. The yield of the product was 17.6 mg, and its estimated purity by LCMS analysis was 92.4%. Analysis condition A: Retention time=1.84 min; ESI-MS (+) m/z [M+H]⁺: 1967.

### Preparation of Example 1208

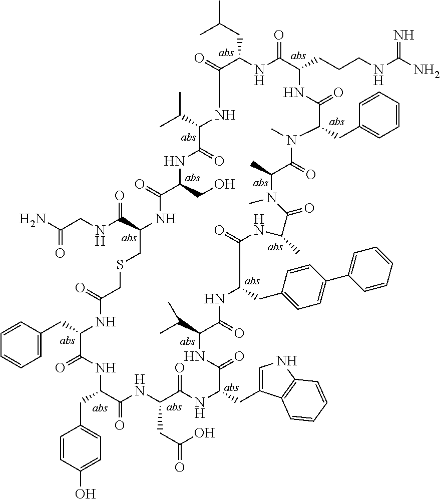 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC1=O]

Example 1208 was prepared on a 50 μmol scale. The yield of the product was 10.9 mg, and its estimated purity by LCMS analysis was 99.3%. Analysis condition A: Retention time=1.81 min; ESI-MS(+) m/z [M+H]⁺: 1923.8.

### Preparation of Example 1209

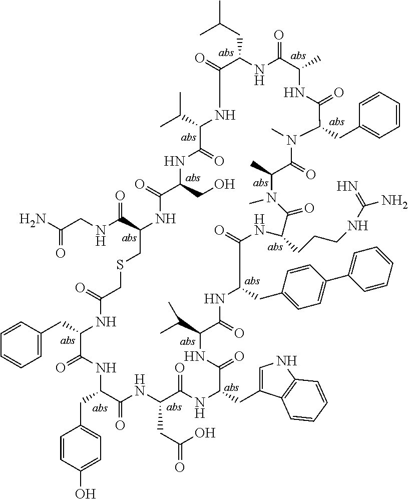 [SMILES: CC(C)C[C@@H]1NC(=O)[C@H](C)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC1=O]

Example 1209 was prepared on a 50 μmol scale. The yield of the product was 20 mg, and its estimated purity by LCMS analysis was 98%. Analysis condition A: Retention time=1.86 min: ESI-MS(+) m/z [M+H]⁺: 1925.1.

### Preparation of Example 1210

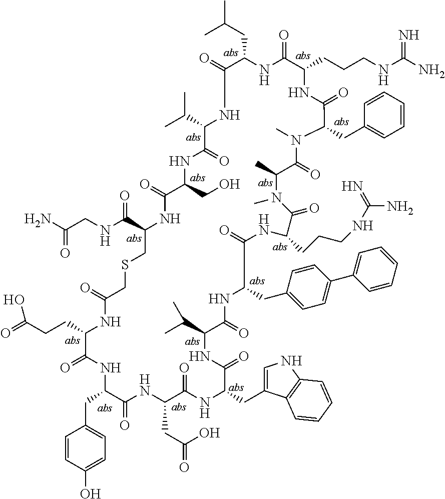 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CCC(=O)O)NC(=O)CSC[C@@H](C(=O)NCC(N)=O)NC(=O)[C@H](CO)NC(=O)[C@H](C(C)C)NC1=O]

Example 1210 was prepared on a 50 μmol scale. The yield of the product was 17 mg, and its estimated purity by LCMS analysis was 96.5%. Analysis condition A: Retention time=1.77 min; ESI-MS(+) m/z [M+H]⁺: 1991.1.

### Preparation of Example 1211

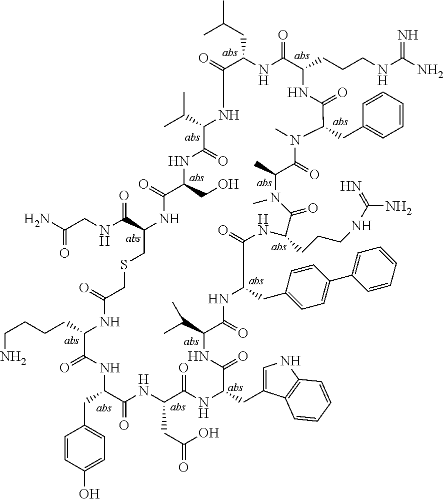 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CCCCN)NC(=O)CSC[C@@H](C(=O)NCC(N)=O)NC(=O)[C@H](CO)NC(=O)[C@H](C(C)C)NC1=O]

Example 1211 was prepared on a 50 μmol scale. The yield of the product was 18.5 mg, and its estimated purity by LCMS analysis was 100%. Analysis condition A: Retention time=2.61 min; ESI-MS(+) m/z [M+H]⁺: 1989.9.

### Preparation of Example 1212

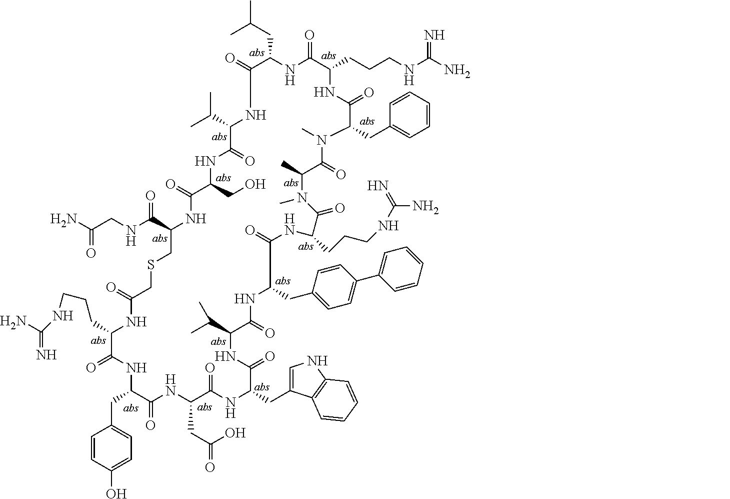 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CCCNC(=N)N)NC(=O)CSC[C@@H](C(=O)NCC(N)=O)NC(=O)[C@H](CO)NC(=O)[C@H](C(C)C)NC1=O]

Example 1212 was prepared on a 50 μmol scale. The yield of the product was 18.7 mg, and its estimated purity by LCMS analysis was 100%. Analysis condition A: Retention time=2.69 min; ESI-MS (+) m/z [M+2H]²⁺: 1010.2.

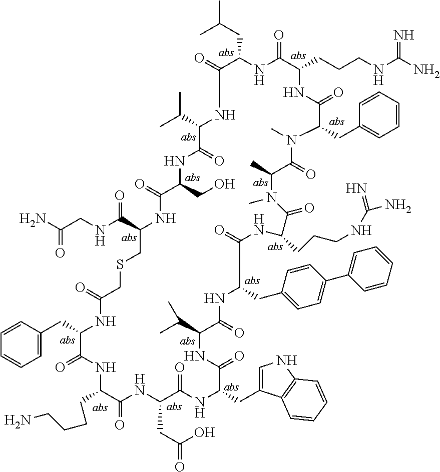 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](CCCCN)NC(=O)[C@H](Cc2ccccc2)NC(=O)CSC[C@@H](C(=O)NCC(N)=O)NC(=O)[C@H](CO)NC(=O)[C@H](C(C)C)NC1=O]

Example 1213 was prepared on a 50 μmol scale. The yield of the product was 22.4 mg, and its estimated purity by LCMS analysis was 94.8%. Analysis condition B: Retention time=1.57 min; ESI-MS(+) m/z [M+H]⁺: 1974.1.

### Preparation of Example 1214

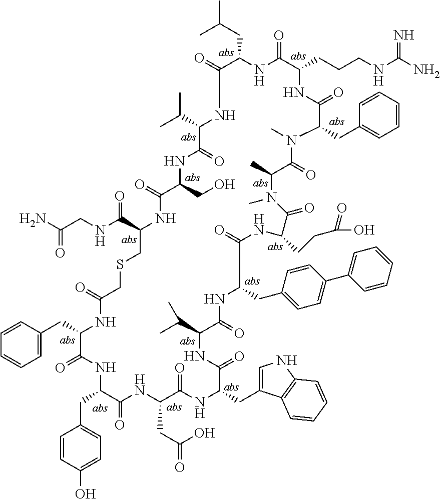 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC1=O]

Example 1214 was prepared on a 50 μmol scale. The yield of the product was 26 mg, and its estimated purity by LCMS analysis was 97.7%. Analysis condition A: Retention time=1.65 min: ESI-MS(+) m/z [M+H]⁺: 1982.

### Preparation of Example 1215

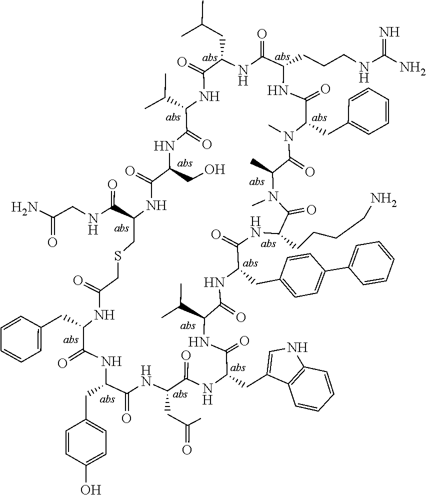 [SMILES: CC(=O)C[C@@H]1NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CCCNC(=N)N)NC(=O)[C@H](Cc2ccccc2)N(C)C(=O)[C@H](C)N(C)C(=O)[C@H](CCCCN)NC(=O)[C@H](Cc2ccc(-c3ccccc3)cc2)NC(=O)[C@H](C(C)C)NC(=O)[C@H](Cc2c[nH]c3ccccc23)NC1=O]

Example 1215 was prepared on a 50 μmol scale. The yield of the product was 30.2 mg, and its estimated purity by LCMS analysis was 99.1%. Analysis condition A: Retention time=1.99 min; ESI-MS(+) m/z [M+H]⁺: 1981.

### Preparation of Example 1216

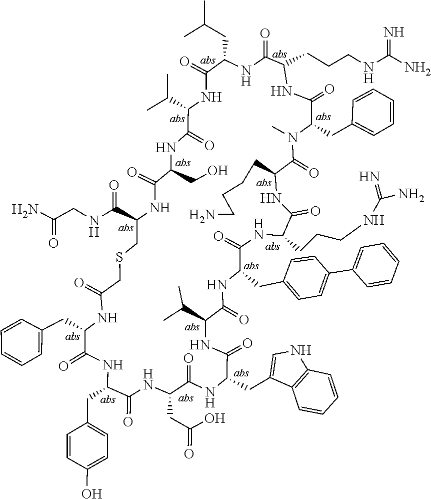

Example 1216 was prepared on a 50 μmol scale. The yield of the product was 9.9 mg, and its estimated purity by LCMS analysis was 93.8%. Analysis condition A: Retention time=1.63 min; ESI-MS(+) m/z [M+2H]²⁺: 1027.

### Preparation of Example 1217

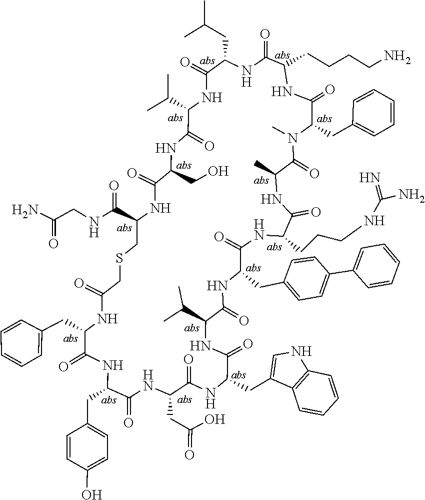

Example 1217 was prepared on a 50 μmol scale. The yield of the product was 19.9 mg, and its estimated purity by LCMS analysis was 99.1%. Analysis condition A: Retention time=1.99 min; ESI-MS(+) m/z [M+H]⁺: 1981.

### Preparation of Example 1218

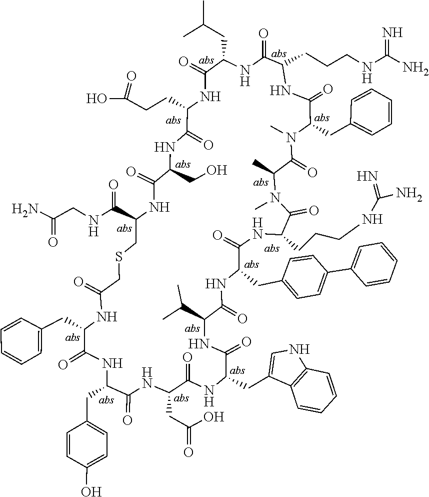

Example 1218 was prepared on a 50 μmol scale. The yield of the product was 26.6 mg, and its estimated purity by LCMS analysis was 96%. Analysis condition A: Retention time=1.77 min; ESI-MS(+) m/z [M+2H]²⁺: 1020.

### Preparation of Example 1219

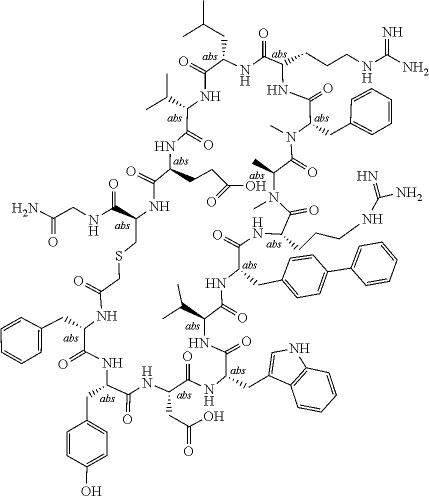

Example 1219 was prepared on a 50 μmol scale. The yield of the product was 9.2 mg, and its estimated purity by LCMS analysis was 100%. Analysis condition B: Retention time=1.7 min; ESI-MS(+) m/z [M+2H]²⁺: 1026.1.

### Preparation of Example 1220

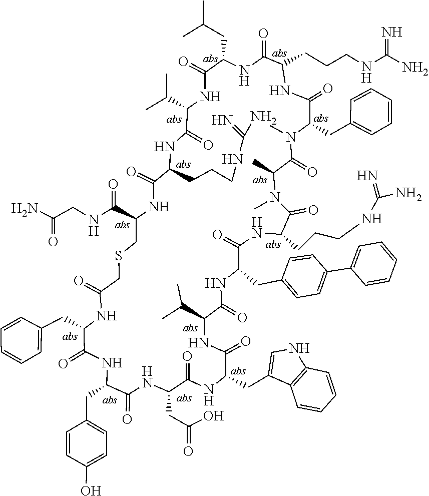

Example 1220 was prepared on a 50 μmol scale. The yield of the product was 33.1 mg, and its estimated purity by LCMS analysis was 100%. Analysis condition B: Retention time=1.61 min; ESI-MS(+) m/z [M+2H]²⁺: 1040.1.

### Preparation of Example 1221

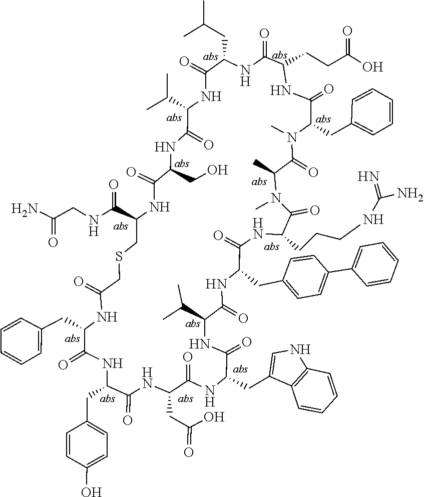

Example 1221 was prepared on a 50 μmol scale. The yield of the product was 6.9 mg, and its estimated purity by LCMS analysis was 95.5%. Analysis condition A: Retention time=1.71 min; ESI-MS(+) m/z [M+H]⁺: 1981.7.

### Preparation of Example 1222

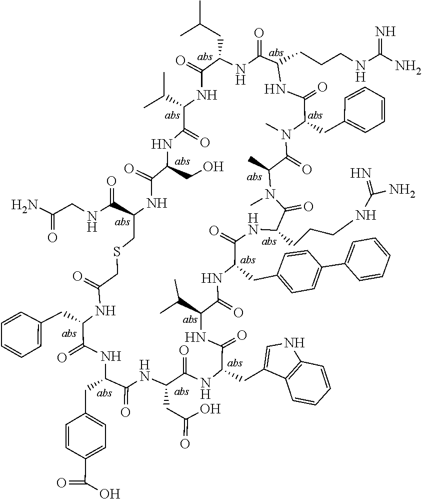

Example 1222 was prepared on a 50 μmol scale. The yield of the product was 25.5 mg, and its estimated purity by LCMS analysis was 91.5%. Analysis condition A: Retention time=1.77 min; ESI-MS(+) m/z [M+2H]²⁺: 1019.1.

### Preparation of Example 1223

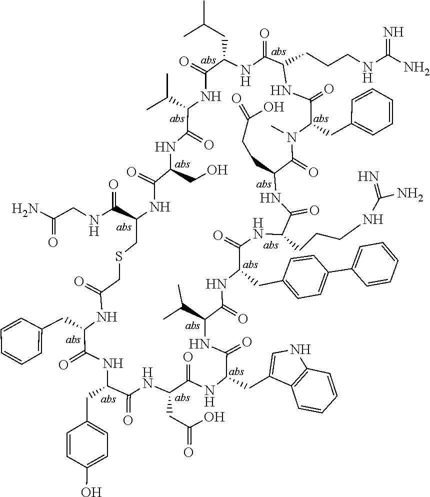

Example 1223 was prepared on a 50 μmol scale. The yield of the product was 24.5 mg, and its estimated purity by LCMS analysis was 100%. Analysis condition A: Retention time=1.71 min; ESI-MS(+) m/z [M+2H]²⁺: 1027.2.

### Preparation of Example 1224

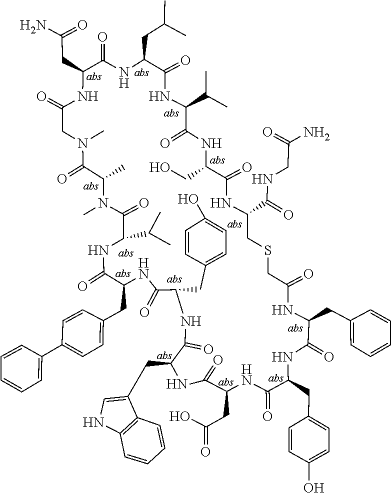 [SMILES: CC(C)C[C@@H]1NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC(=O)[C@H](Cc2ccc(O)cc2)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC1=O]

Example 1224 was prepared on a 50 μmol scale. The yield of the product was 27.6 mg, and its estimated purity by LCMS analysis was 100%. Analysis condition A: Retention time=1.54 min; ESI-MS(+) m/z [M+H]⁺: 1884.9.

### Preparation of Example 1225

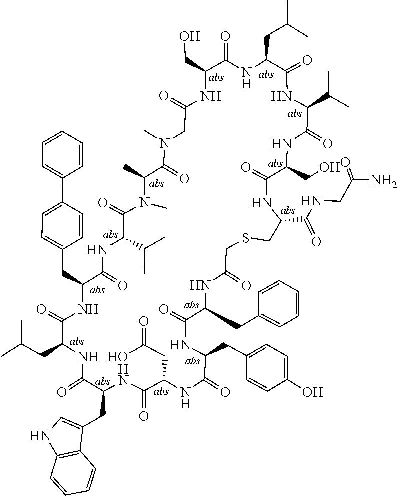 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CO)NC(=O)CN(C)C(=O)[C@H](C)N(C)C(=O)[C@H](C(C)C)NC(=O)[C@H](Cc2ccc(-c3ccccc3)cc2)NC1=O]

Example 1225 was prepared on a 50 μmol scale. The yield of the product was 16.2 mg, and its estimated purity by LCMS analysis was 97.4%. Analysis condition A: Retention time=1.65 min; ESI-MS(+) m/z [M+H]⁺: 1807.1.

### Preparation of Example 1226

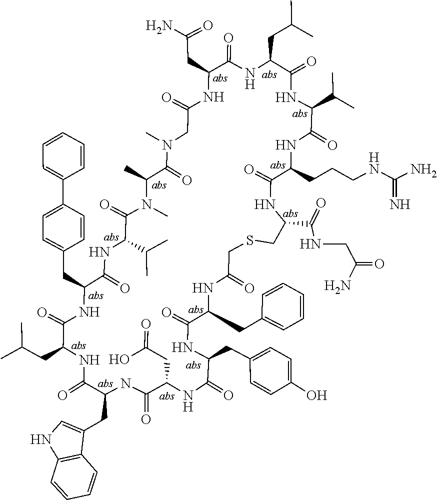

Example 1226 was prepared on a 50 μmol scale. The yield of the product was 39 mg, and its estimated purity by LCMS analysis was 90.1%. Analysis condition B: Retention time=1.72 min; ESI-MS(+) m/z [M+H]⁺: 1904.

### Preparation of Example 1227

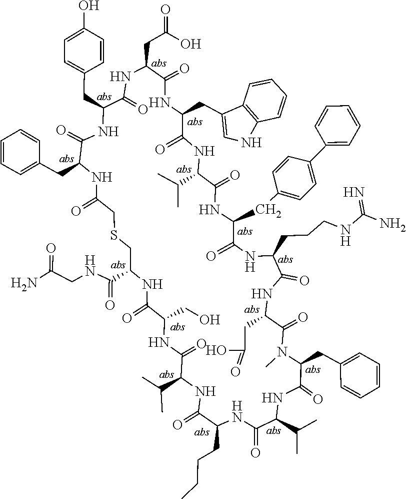 [SMILES: CCCC[C@@H]1NC(=O)[C@H](C(C)C)NC(=O)[C@H](Cc2ccccc2)N(C)C(=O)[C@H](CC(=O)O)NC(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC1=O]

Example 1227 was prepared on a 50 μmol scale. The yield of the product was 3.7 mg, and its estimated purity by LCMS analysis was 96.7%. Analysis condition A: Retention time=1.64 min; ESI-MS(+) m/z [M+H]⁺: 1981.8.

### Preparation of Example 1228

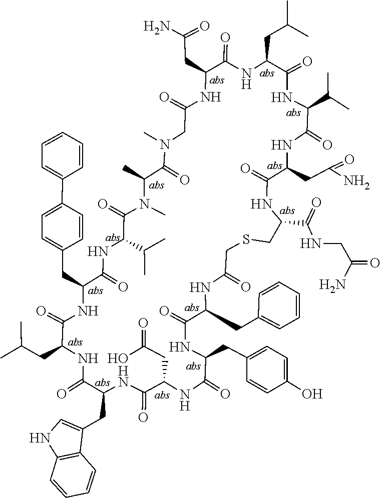 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CC(N)=O)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1228 was prepared on a 50 μmol scale. The yield of the product was 30.8 mg, and its estimated purity by LCMS analysis was 98.1%. Analysis condition B: Retention time=1.84 min: ESI-MS(+) m/z [M+H]⁺: 1860.6.

### Preparation of Example 1229

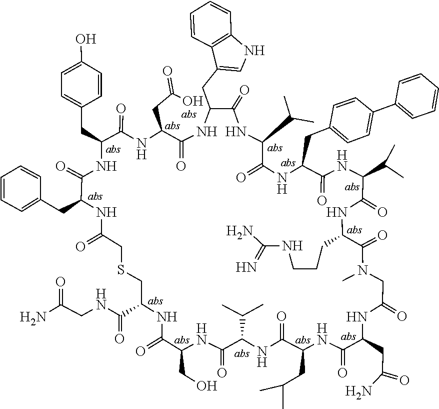 [SMILES: CC(C)C[C@@H]1NC(=O)[C@H](CC(N)=O)NC(=O)CN(C)C(=O)[C@H](CCCNC(=N)N)NC(=O)[C@H](C(C)C)NC(=O)[C@H](Cc2ccc(-c3ccccc3)cc2)NC(=O)[C@H](C(C)C)NC(=O)C(Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC1=O]

Example 1229 was prepared on a 50 μmol scale. The yield of the product was 36.6 mg, and its estimated purity by LCMS analysis was 100%. Analysis condition A: Retention time=1.59 min; ESI-MS(+) m/z [M+2H]²⁺: 946.1.

### Preparation of Example 1230

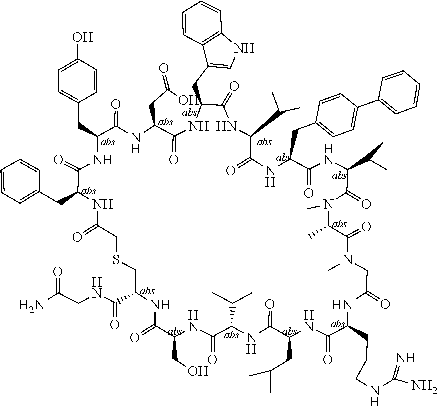

Example 1230 was prepared on a 50 μmol scale. The yield of the product was 14.5 mg, and its estimated purity by LCMS analysis was 98.9%. Analysis condition B: Retention time=1.59 min; ESI-MS(+) m/z [M+2H]²⁺: 932.1.

### Preparation of Example 1231

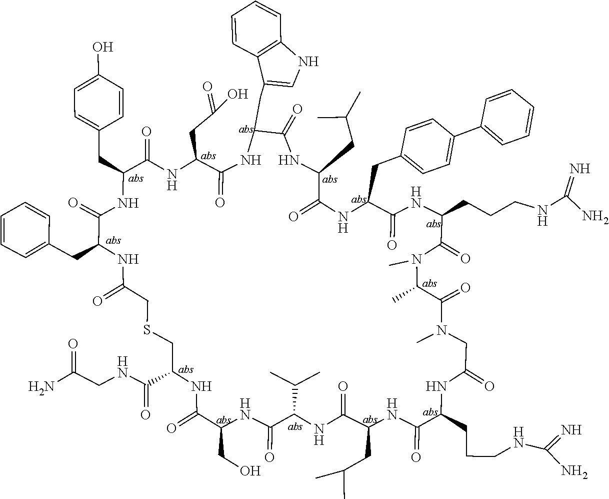 [SMILES: CC(C)C[C@@H]1NC(=O)C(Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CCCNC(=N)N)NC(=O)CN(C)C(=O)[C@H](C)N(C)C(=O)[C@H](CCCNC(=N)N)NC(=O)[C@H](Cc2ccc(-c3ccccc3)cc2)NC1=O]

Example 1231 was prepared on a 50 μmol scale. The yield of the product was 40.6 mg, and its estimated purity by LCMS analysis was 96.5%. Analysis condition A: Retention time=1.55, 1.63 min; ESI-MS(+) m/z [M+2H]²⁺: 967.7.

### Preparation of Example 1232

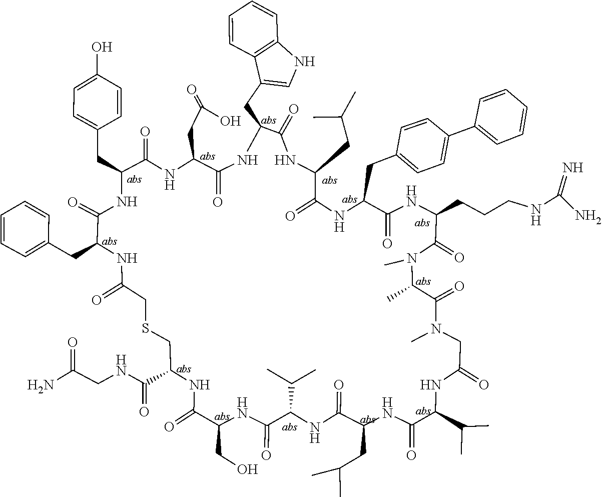 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](C(C)C)NC(=O)CN(C)C(=O)[C@H](C)N(C)C(=O)[C@H](CCCNC(=N)N)NC(=O)[C@H](Cc2ccc(-c3ccccc3)cc2)NC1=O]

Example 1232 was prepared on a 50 μmol scale. The yield of the product was 15.6 mg, and its estimated purity by LCMS analysis was 98%. Analysis condition B: Retention time=1.67 min; ESI-MS(+) m/z [M+2H]²⁺: 939.

### Preparation of Example 1233

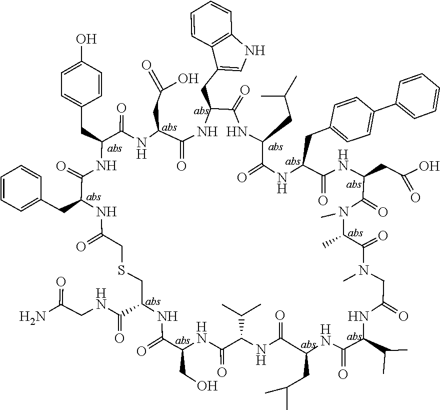 [SMILES: CC(C)C[C@H]1CC(=O)N[C@@H](C(C)C)C(=O)N[C@@H](CO)CC(=O)N[C@H](C(=O)NCC(N)=O)CSCC(=O)N[C@@H](Cc2ccccc2)C(=O)N[C@@H](Cc2ccc(O)cc2)C(=O)N[C@@H](CC(=O)O)C(=O)N[C@@H](Cc2c[nH]c3ccccc23)C(=O)N[C@@H](CC(C)C)C(=O)N[C@@H](Cc2ccc(-c3ccccc3)cc2)C(=O)N[C@@H](CC(=O)O)C(=O)N(C)[C@@H](C)C(=O)N(C)CC(=O)N[C@@H](C(C)C)C(=O)N1]

Example 1233 was prepared on a 50 μmol scale. The yield of the product was 24.1 mg, and its estimated purity by LCMS analysis was 97.9%. Analysis condition B: Retention time—1.65, 1.7 min; ESI-MS(+) m/z [M+2H]²⁺: 918.06, 918.06.

### Preparation of Example 1234

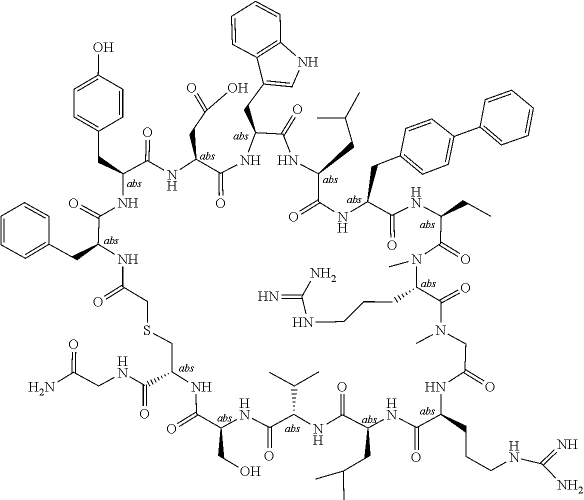 [SMILES: CC[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CCCNC(=N)N)NC(=O)CN(C)C(=O)[C@H](CCCNC(=N)N)N(C)C1=O]

Example 1234 was prepared on a 50 μmol scale. The yield of the product was 32.5 mg, and its estimated purity by LCMS analysis was 96.2%. Analysis condition B: Retention time=1.6 min; ESI-MS(+) m/z [M+3H]³⁺: 650.

### Preparation of Example 1235

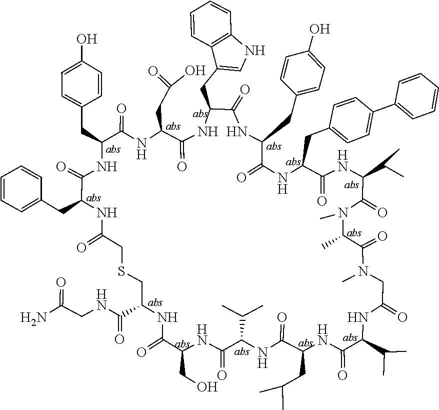 [SMILES: CC(C)C[C@@H]1NC(=O)[C@H](C(C)C)NC(=O)CN(C)C(=O)[C@H](C)N(C)C(=O)[C@H](C(C)C)NC(=O)[C@H](Cc2ccc(-c3ccccc3)cc2)NC(=O)[C@H](Cc2ccc(O)cc2)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC1=O]

Example 1235 was prepared on a 50 μmol scale. The yield of the product was 13.9 mg, and its estimated purity by LCMS analysis was 98.1%. Analysis condition A: Retention time=1.56 min; ESI-MS(+) m/z [M+2H]²⁺: 935.

### Preparation of Example 1236

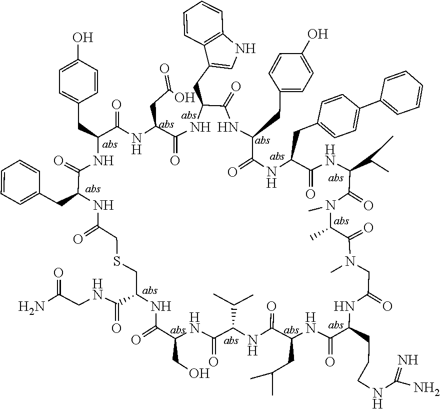 [SMILES: CC(C)C[C@@H]1NC(=O)[C@H](CCCNC(=N)N)NC(=O)CN(C)C(=O)[C@H](C)N(C)C(=O)[C@H](C(C)C)NC(=O)[C@H](Cc2ccc(-c3ccccc3)cc2)NC(=O)[C@H](Cc2ccc(O)cc2)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC1=O]

Example 1236 was prepared on a 50 μmol scale. The yield of the product was 34.9 mg, and its estimated purity by LCMS analysis was 99.1%. Analysis condition A: Retention time=1.47 min; ESI-MS(+) m/z [M+2H]²⁺: 964.1.

### Preparation of Example 1237

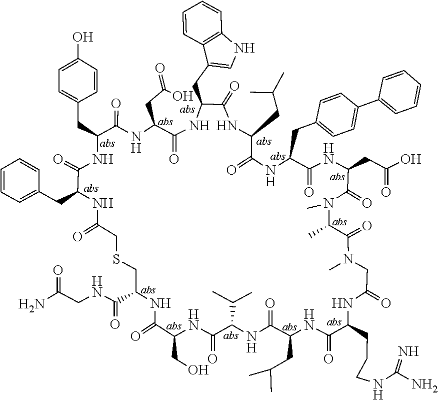 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CCCNC(=N)N)NC(=O)CN(C)C(=O)[C@H](C)N(C)C(=O)[C@H](CC(=O)O)NC(=O)[C@H](Cc2ccc(-c3ccccc3)cc2)NC1=O]

Example 1237 was prepared on a 50 μmol scale. The yield of the product was 37.1 mg, and its estimated purity by LCMS analysis was 96.3%. Analysis condition B: Retention time=1.66 min; ESI-MS(+)m/z [M+2H]²⁺: 946.1.

### Preparation of Example 1238

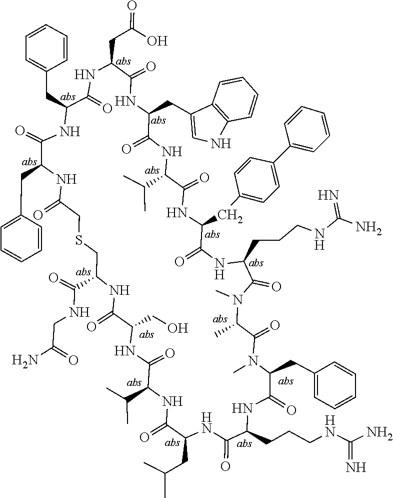 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccccc2)NC(=O)[C@H](Cc2ccccc2)NC(=O)CSC[C@@H](C(=O)NCC(N)=O)NC(=O)[C@H](CO)NC(=O)[C@H](C(C)C)NC1=O]

Example 1238 was prepared on a 50 μmol scale. The yield of the product was 28.9 mg, and its estimated purity by LCMS analysis was 91.9%. Analysis condition A: Retention time=1.89 min; ESI-MS(+) m/z [M+2H]²⁺: 997.1.

### Preparation of Example 1239

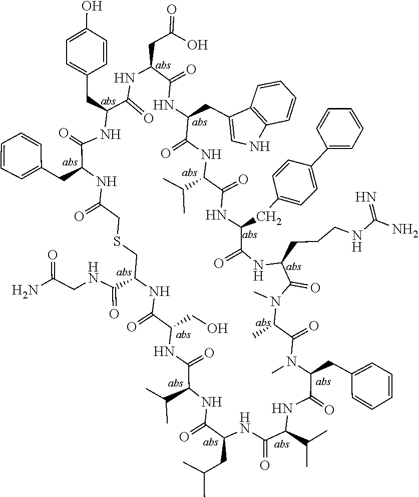 [SMILES: CC(C)C[C@@H]1NC(=O)[C@H](C(C)C)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC1=O]

Example 1239 was prepared on a 50 μmol scale. The yield of the product was 3.7 mg, and its estimated purity by LCMS analysis was 92.9%. Analysis condition A: Retention time=1.81, 1.85 min; ESI-MS(+) m/z [M+2H]²⁺: 977.18, 977.18.

### Preparation of Example 1240

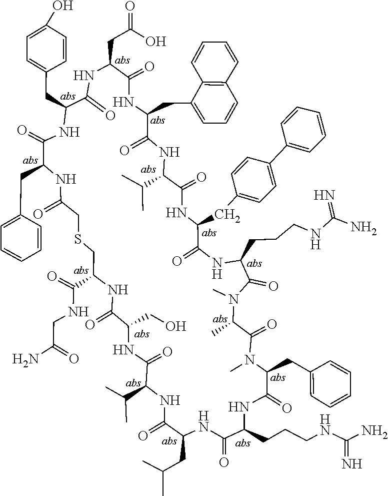 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2cccc3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC1=O]

Example 1240 was prepared on a 50 μmol scale. The yield of the product was 34.2 mg, and its estimated purity by LCMS analysis was 98.6%. Analysis condition B: Retention time=1.89 min; ESI-MS(+) m/z [M+2H]²⁺: 1011.1.

### Preparation of Example 1241

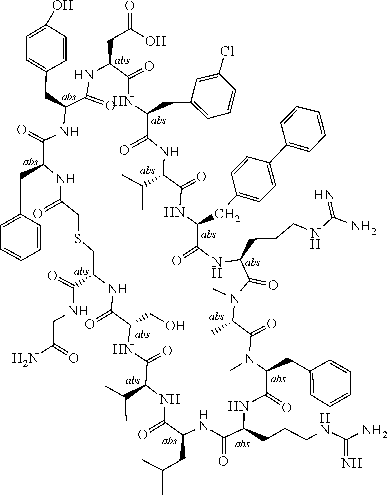 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2cccc(Cl)c2)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC1=O]

Example 1241 was prepared on a 50 μmol scale. The yield of the product was 30.4 mg, and its estimated purity by LCMS analysis was 98.5%. Analysis condition B: Retention time=1.86 min; ESI-MS(+) m/z [M+2H]²⁺: 1003.1.

### Preparation of Example 1242

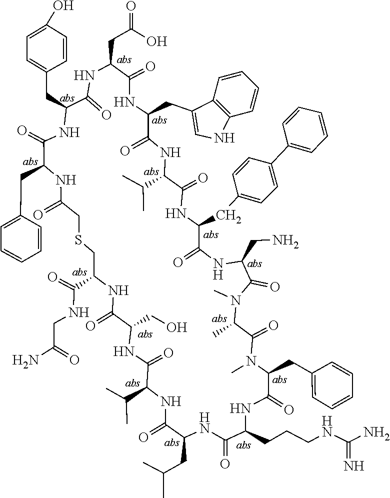 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CN)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC1=O]

Example 1242 was prepared on a 50 μmol scale. The yield of the product was 37.6 mg, and its estimated purity by LCMS analysis was 100%. Analysis condition A: Retention time=2.08 min; ESI-MS(+) m/z [M+H]⁺: 1938.7.

### Preparation of Example 1243

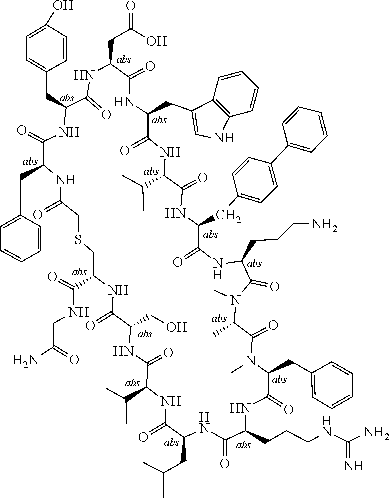 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCN)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC1=O]

Example 1243 was prepared on a 50 μmol scale. The yield of the product was 53.1 mg, and its estimated purity by LCMS analysis was 100%. Analysis condition A: Retention time=1.81 min; ESI-MS(+) m/z [M+H]⁺: 1967.

### Preparation of Example 1244

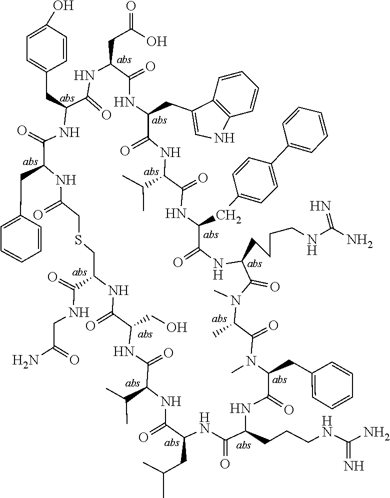 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC1=O]

Example 1244 was prepared on a 50 μmol scale. The yield of the product was 53.2 mg, and its estimated purity by LCMS analysis was 98.5%. Analysis condition A: Retention time=1.78 min; ESI-MS(+) m/z [M+2H]²⁺: 1012.2.

### Preparation of Example 1245

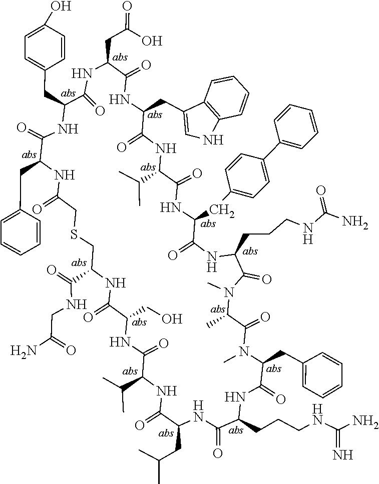 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC1=O]

Example 1245 was prepared on a 50 μmol scale. The yield of the product was 3.7 mg, and its estimated purity by LCMS analysis was 91.5%. Analysis condition A: Retention time=1.69 min: ESI-MS(+) m/z [M+2H]²⁺: 1005.4.

### Preparation of Example 1246

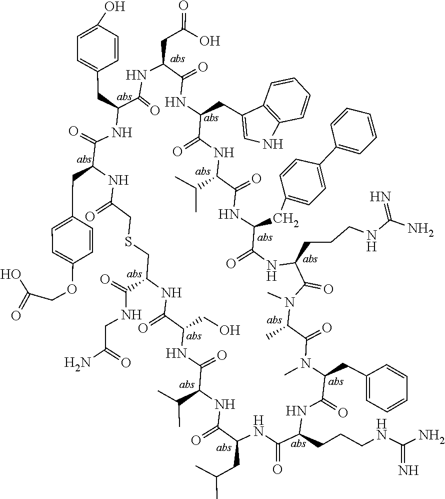 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccc(OCC(=O)O)cc2)NC(=O)CSC[C@@H](C(=O)NCC(N)=O)NC(=O)[C@H](CO)NC(=O)[C@H](C(C)C)NC1=O]

Example 1246 was prepared on a 50 μmol scale. The yield of the product was 24.8 mg, and its estimated purity by LCMS analysis was 89%. Analysis condition B: Retention time=1.52, 1.61 min; ESI-MS(+) m/z [M+2H]²⁺: 1042.9.

### Preparation of Example 1247

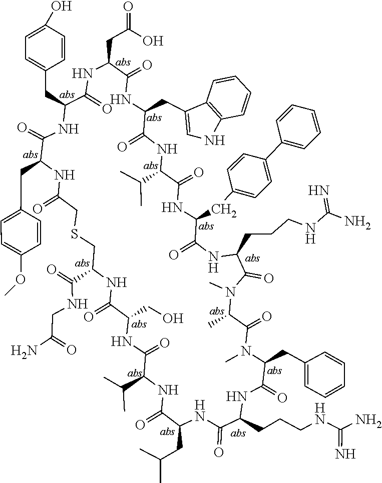 [SMILES: COc1ccc(C[C@@H]2NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CCCNC(=N)N)NC(=O)[C@H](Cc3ccccc3)N(C)C(=O)[C@H](C)N(C)C(=O)[C@H](CCCNC(=N)N)NC(=O)[C@H](Cc3ccc(-c4ccccc4)cc3)NC(=O)[C@H](C(C)C)NC(=O)[C@H](Cc3c[nH]c4ccccc34)NC(=O)[C@H](CC(=O)O)NC(=O)[C@H](Cc3ccc(O)cc3)NC2=O)cc1]

Example 1247 was prepared on a 50 μmol scale. The yield of the product was 33.6 mg, and its estimated purity by LCMS analysis was 97.1%. Analysis condition A: Retention time=1.78 min; ESI-MS(+) m/z [M+2H]²⁺: 1020.3.

### Preparation of Example 1248

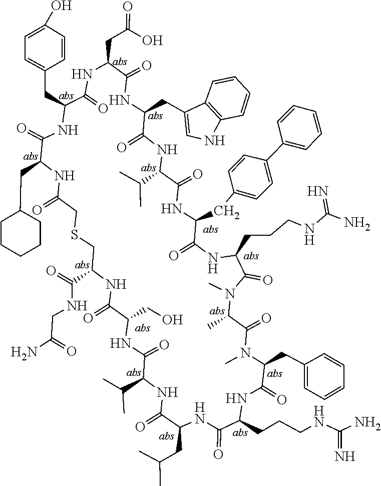 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC2CCCCC2)NC(=O)CSC[C@@H](C(=O)NCC(N)=O)NC(=O)[C@H](CO)NC(=O)[C@H](C(C)C)NC1=O]

Example 1248 was prepared on a 50 μmol scale. The yield of the product was 14.8 mg, and its estimated purity by LCMS analysis was 91%. Analysis condition B: Retention time=1.56, 1.78 min: ESI-MS(+) m/z [M+2H]²⁺: 672.4.

### Preparation of Example 1249

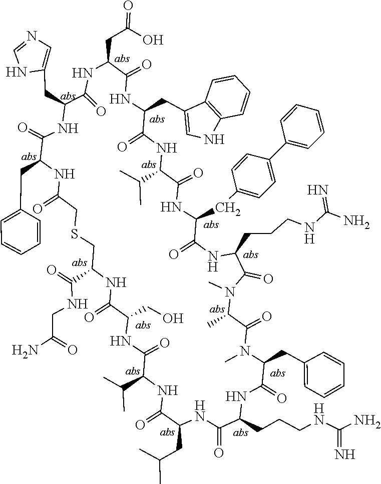 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2cnc[nH]2)NC(=O)[C@H](Cc2ccccc2)NC(=O)CSC[C@@H](C(=O)NCC(N)=O)NC(=O)[C@H](CO)NC(=O)[C@H](C(C)C)NC1=O]

Example 1249 was prepared on a 50 μmol scale. The yield of the product was 42 mg, and its estimated purity by LCMS analysis was 96.5%. Analysis condition A: Retention time=1.73 min; ESI-MS(+) m/z [M+3H]³⁺: 661.9.

### Preparation of Example 1250

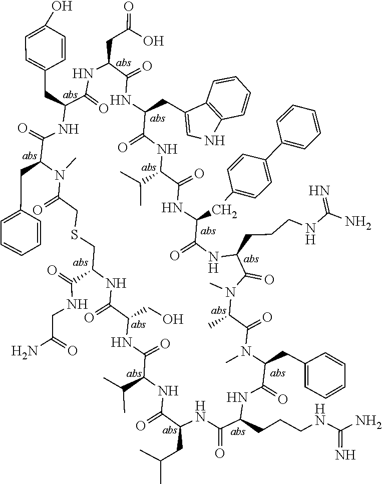 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)N(C)C(=O)CSC[C@@H](C(=O)NCC(N)=O)NC(=O)[C@H](CO)NC(=O)[C@H](C(C)C)NC1=O]

Example 1250 was prepared on a 50 μmol scale. The yield of the product was 18.6 mg, and its estimated purity by LCMS analysis was 97.4%. Analysis condition B: Retention time=1.69 min; ESI-MS(+) m/z [M+3H]³⁺: 675.2.

### Preparation of Example 1251

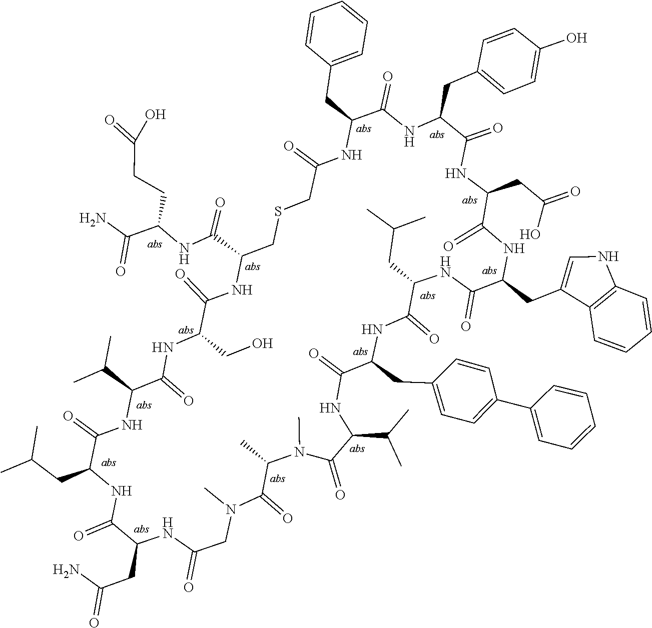 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](CCC(=O)O)C(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1251 was prepared on a 50 μmol scale. The yield of the product was 29.3 mg, and its estimated purity by LCMS analysis was 98.5%. Analysis condition B: Retention time=1.74 min; ESI-MS(+) m/z [M+2H]²⁺: 954.1.

### Preparation of Example 1252

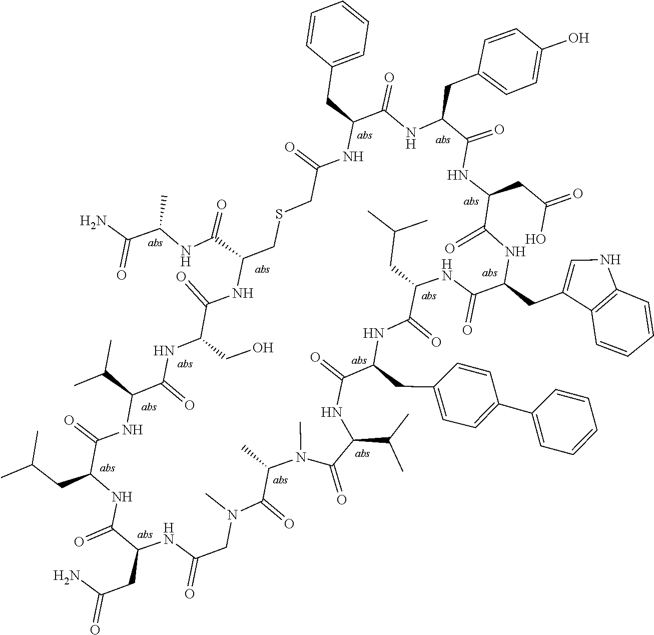 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1252 was prepared on a 50 μmol scale. The yield of the product was 24 mg, and its estimated purity by LCMS analysis was 98.8%. Analysis condition A: Retention time=1.58 min; ESI-MS(+) m/z [M+2H]²⁺: 925.2.

### Preparation of Example 1253

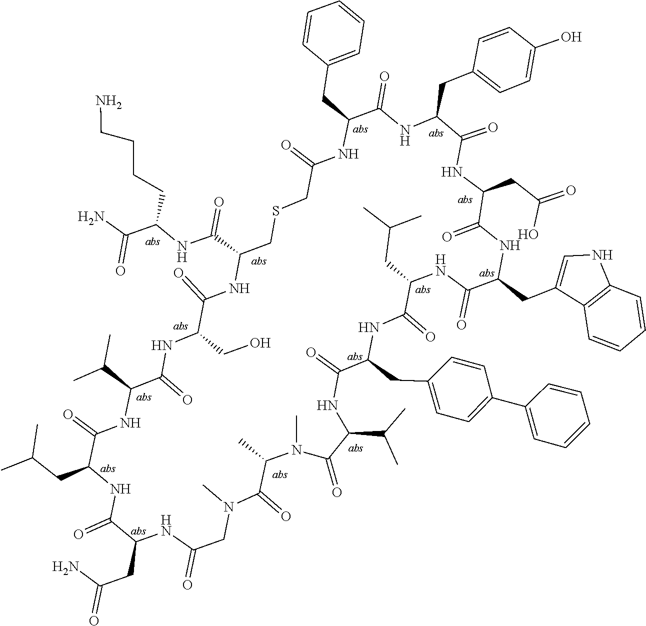 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](CCCCN)C(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1253 was prepared on a 50 μmol scale. The yield of the product was 16.3 mg, and its estimated purity by LCMS analysis was 95.5%. Analysis condition B: Retention time=1.69 min; ESI-MS(+) m/z [M+2H]²⁺: 953.1.

### Preparation of Example 1254

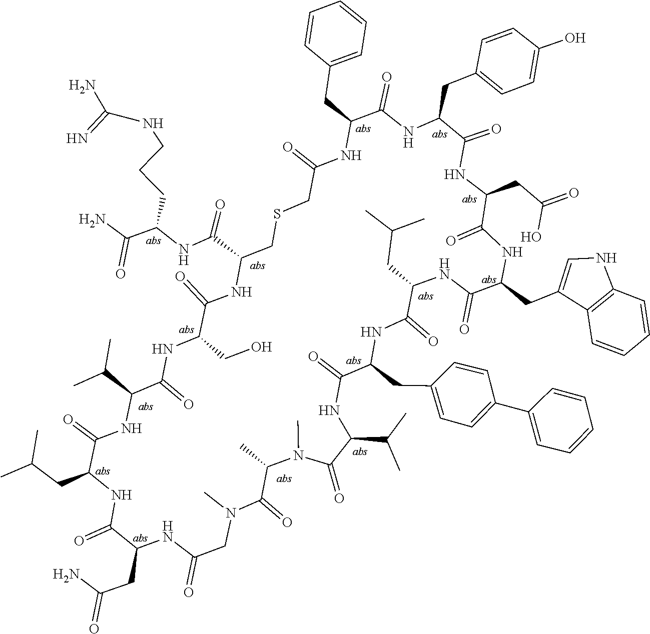 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](CCCNC(=N)N)C(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc2ccc(-c3ccccc3)cc2)NC1=O]

Example 1254 was prepared on a 50 μmol scale. The yield of the product was 16.3 mg, and its estimated purity by LCMS analysis was 98.5%. Analysis condition A: Retention time=1.59 min; ESI-MS(+) m/z [M+2H]²⁺: 967.2.

### Preparation of Example 1255

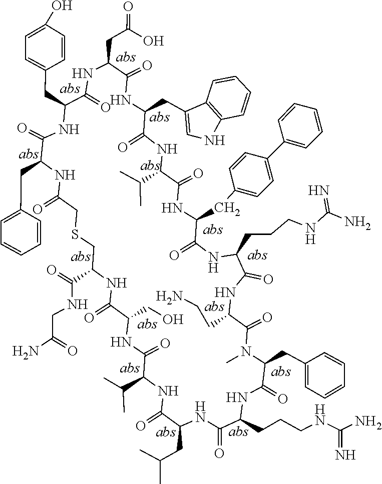 [SMILES: CC(C)C[C@@H]1NC(=O)[C@H](CCCNC(=N)N)NC(=O)[C@H](Cc2ccccc2)N(C)C(=O)[C@H](CCN)NC(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC1=O]

Example 1255 was prepared on a 50 μmol scale. The yield of the product was 12.9 mg, and its estimated purity by LCMS analysis was 92.3%. Analysis condition A: Retention time=1.56 min; ESI-MS(+) m/z [M+2H]²⁺: 1013.1.

### Preparation of Example 1256

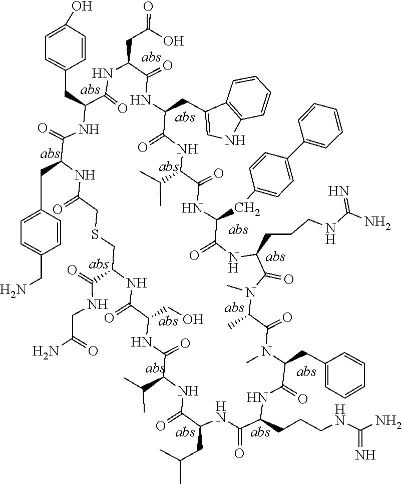 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccc(CN)cc2)NC(=O)CSC[C@@H](C(=O)NCC(N)=O)NC(=O)[C@H](CO)NC(=O)[C@H](C(C)C)NC1=O]

Example 1256 was prepared on a 50 μmol scale. The yield of the product was 19.9 mg, and its estimated purity by LCMS analysis was 96%. Analysis condition A: Retention time=1.58 min; ESI-MS(+) m/z [M+3H]³⁺: 680.

### Preparation of Example 1257

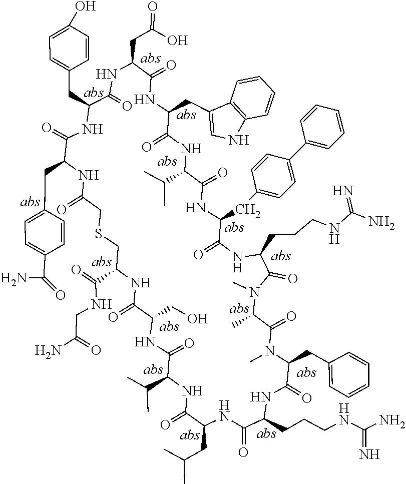 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccc(C(N)=O)cc2)NC(=O)CSC[C@@H](C(=O)NCC(N)=O)NC(=O)[C@H](CO)NC(=O)[C@H](C(C)C)NC1=O]

Example 1257 was prepared on a 50 μmol scale. The yield of the product was 30.1 mg, and its estimated purity by LCMS analysis was 95.7%. Analysis condition A: Retention time=1.65 min; ESI-MS(+) m/z [M+2H]²⁺: 1027.

### Preparation of Example 1258

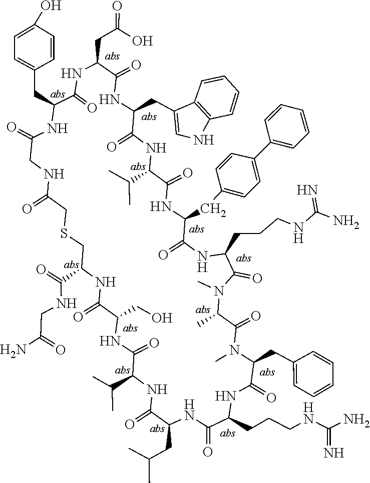 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)CNC(=O)CSC[C@@H](C(=O)NCC(N)=O)NC(=O)[C@H](CO)NC(=O)[C@H](C(C)C)NC1=O]

Example 1258 was prepared on a 50 μmol scale. The yield of the product was 35.7 mg, and its estimated purity by LCMS analysis was 99%. Analysis condition A: Retention time=1.97 min: ESI-MS(+) m/z [M+H]⁺: 1918.9.

### Preparation of Example 1259

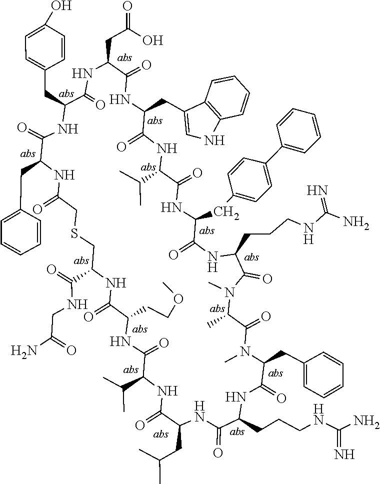 [SMILES: COCC[C@@H]1NC(=O)[C@H](C(C)C)NC(=O)[C@H](CC(C)C)NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC1=O]

Example 1259 was prepared on a 50 μmol scale. The yield of the product was 28.5 mg, and its estimated purity by LCMS analysis was 100%. Analysis condition A: Retention time=1.85 min ESI-MS(+) m/z [M+2H]⁺: 1019.8.

### Preparation of Example 1260

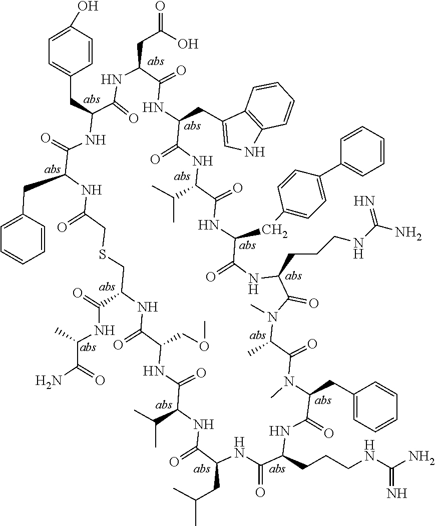 [SMILES: COC[C@@H]1NC(=O)[C@H](C(C)C)NC(=O)[C@H](CC(C)C)NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC1=O]

Example 1260 was prepared on a 50 μmol scale. The yield of the product was 61.3 mg, and its estimated purity by LCMS analysis was 90.6%. Analysis condition B: Retention time=1.8 min; ESI-MS(+) m/z [M+2H]²⁺: 1012.2.

### Preparation of Example 1261

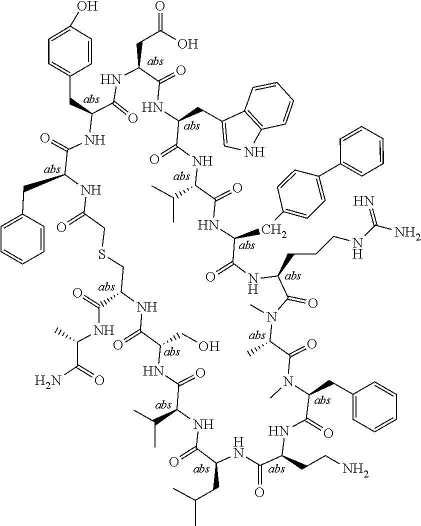 [SMILES: CC(C)C[C@@H]1NC(=O)[C@H](CCN)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC1=O]

Example 1261 was prepared on a 50 μmol scale. The yield of the product was 17.2 mg, and its estimated purity by LCMS analysis was 94.9%. Analysis condition B: Retention time=1.77 min; ESI-MS(+) m/z [M+H]⁺: 1954.

### Preparation of Example 1262

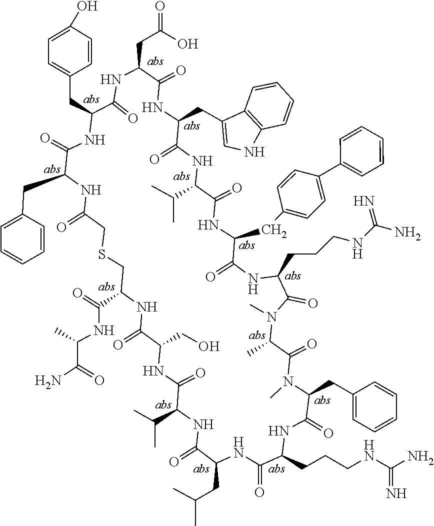 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC1=O]

Example 1262 was prepared on a 50 μmol scale. The yield of the product was 11.7 mg, and its estimated purity by LCMS analysis was 94.8%. Analysis condition A: Retention time=1.69 min; ESI-MS(+) m/z [M+2H]²⁺: 1006.1.

### Preparation of Example 1263

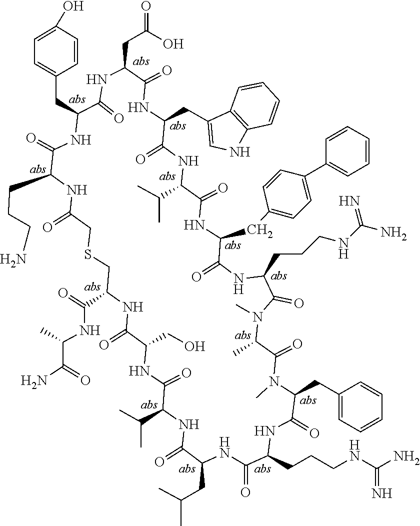 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CCCN)NC(=O)CSC[C@@H](C(=O)N[C@@H](C)C(N)=O)NC(=O)[C@H](CO)NC(=O)[C@H](C(C)C)NC1=O]

Example 1263 was prepared on a 50 μmol scale. The yield of the product was 25.5 mg, and its estimated purity by LCMS analysis was 96.8%. Analysis condition B: Retention time=1.52 min: ESI-MS(+) m/z [M+3H]³⁺: 659.3.

### Preparation of Example 1264

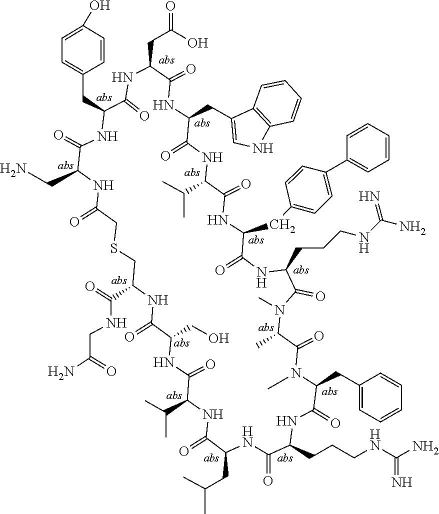 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CN)NC(=O)CSC[C@@H](C(=O)NCC(N)=O)NC(=O)[C@H](CO)NC(=O)[C@H](C(C)C)NC1=O]

Example 1264 was prepared on a 50 μmol scale. The yield of the product was 10 mg, and its estimated purity by LCMS analysis was 99.1%. Analysis condition B: Retention time=1.47 min; ESI-MS(+) m/z [M+3H]³⁺: 650.1.

### Preparation of Example 1265

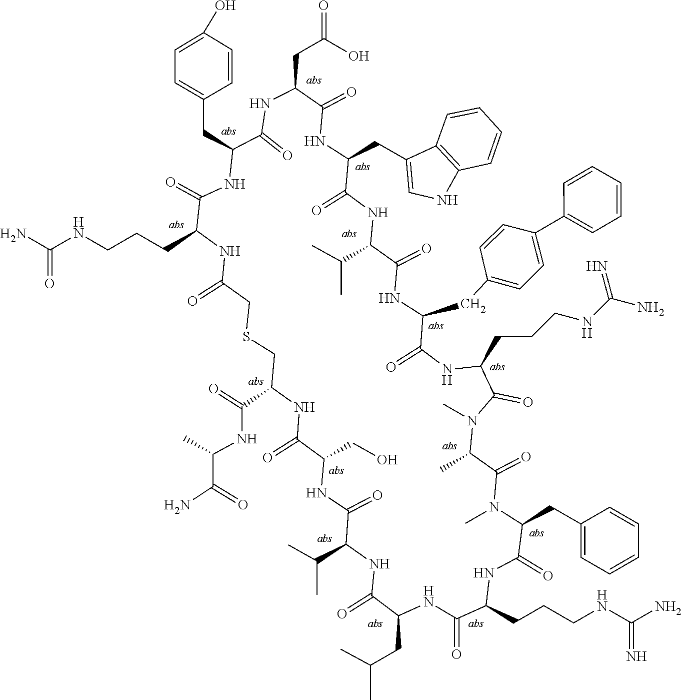 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CCCNC(N)=O)NC(=O)CSC[C@@H](C(=O)N[C@@H](C)C(N)=O)NC(=O)[C@H](CO)NC(=O)[C@H](C(C)C)NC1=O]

Example 1265 was prepared on a 50 μmol scale. The yield of the product was 5.2 mg, and its estimated purity by LCMS analysis was 98.5%. Analysis condition A: Retention time=1.68 min; ESI-MS(+) m/z [M+2H]²⁺: 1010.2.

### Preparation of Example 1266

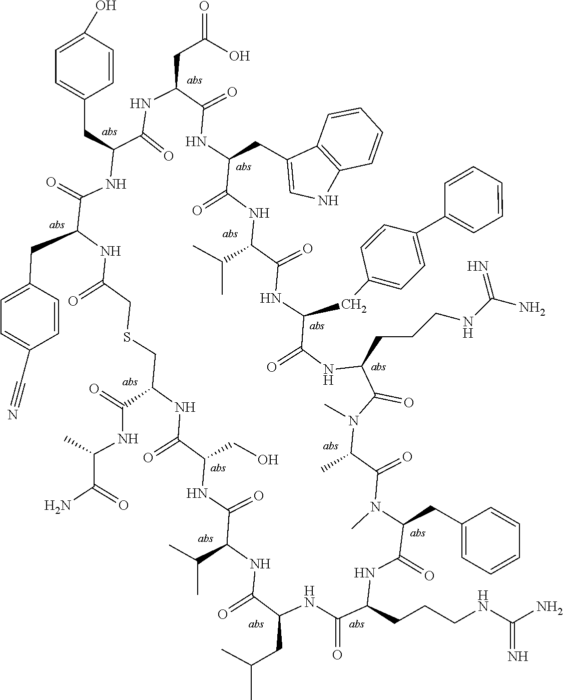 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccc(C#N)cc2)NC(=O)CSC[C@@H](C(=O)N[C@@H](C)C(N)=O)NC(=O)[C@H](CO)NC(=O)[C@H](C(C)C)NC1=O]

Example 1266 was prepared on a 50 μmol scale. The yield of the product was 33.5 mg, and its estimated purity by LCMS analysis was 100%. Analysis condition A: Retention time=1.68, 1.74 min; ESI-MS(+) m/z [M+2H]²⁺: 1017.28, 1017.16.

### Preparation of Example 1267

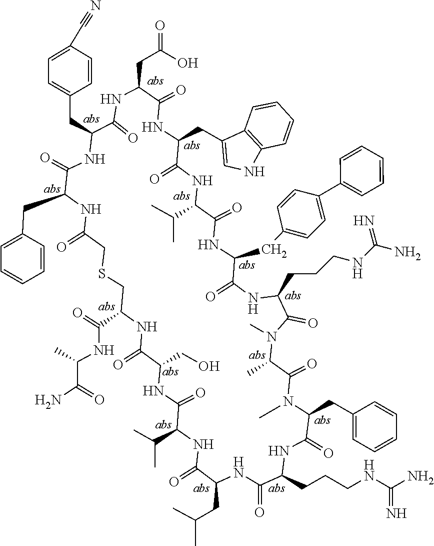 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C#N)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC1=O]

Example 1267 was prepared on a 50 μmol scale. The yield of the product was 34.2 mg, and its estimated purity by LCMS analysis was 97.3%. Analysis condition A: Retention time=1.87 min; ESI-MS(+) m/z [M+2H]²⁺: 1009.1.

### Preparation of Example 1268

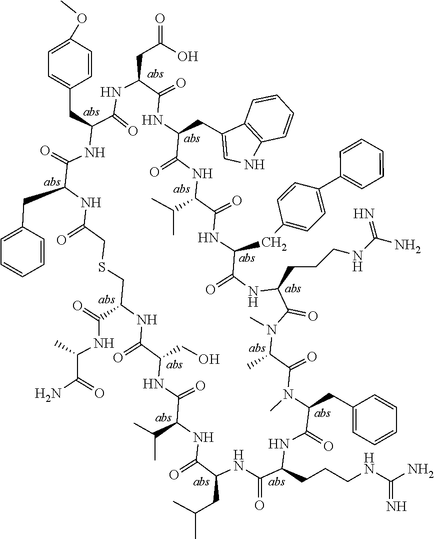 [SMILES: COc1ccc(C[C@@H]2NC(=O)[C@H](Cc3ccccc3)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CCCNC(=N)N)NC(=O)[C@H](Cc3ccccc3)N(C)C(=O)[C@H](C)N(C)C(=O)[C@H](CCCNC(=N)N)NC(=O)[C@H](Cc3ccc(-c4ccccc4)cc3)NC(=O)[C@H](C(C)C)NC(=O)[C@H](Cc3c[nH]c4ccccc34)NC(=O)[C@H](CC(=O)O)NC2=O)cc1]

Example 1268 was prepared on a 50 μmol scale. The yield of the product was 33.5 mg, and its estimated purity by LCMS analysis was 98.9%. Analysis condition A: Retention time=1.91 min; ESI-MS(+) m/z [M+2H]²⁺: 1012.1.

### Preparation of Example 1269

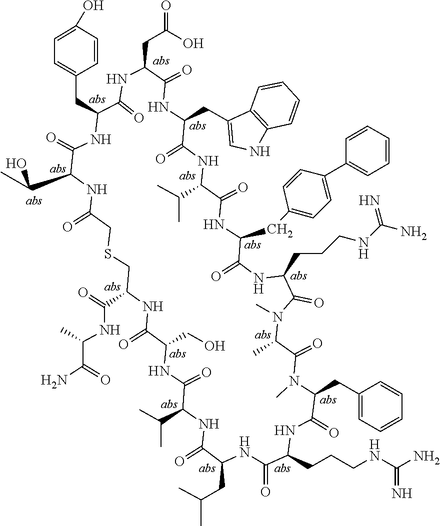 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H]([C@H](C)O)NC(=O)CSC[C@@H](C(=O)N[C@@H](C)C(N)=O)NC(=O)[C@H](CO)NC(=O)[C@H](C(C)C)NC1=O]

Example 1269 was prepared on a 13 μmol scale. The yield of the product was 26.1 mg, and its estimated purity by LCMS analysis was 97.9%. Analysis condition B: Retention time=1.63 min: ESI-MS(+) m/z [M+2H]⁺: 982.2.

### Preparation of Example 1270

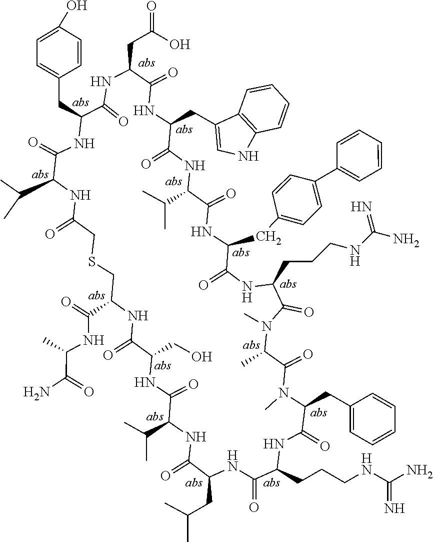 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](C(C)C)NC(=O)CSC[C@@H](C(=O)N[C@@H](C)C(N)=O)NC(=O)[C@H](CO)NC(=O)[C@H](C(C)C)NC1=O]

Example 1270 was prepared on a 50 μmol scale. The yield of the product was 30.1 mg, and its estimated purity by LCMS analysis was 100%. Analysis condition A: Retention time=1.72 min: ESI-MS(+) m/z [M+2H]⁺: 981.2.

### Preparation of Example 1271

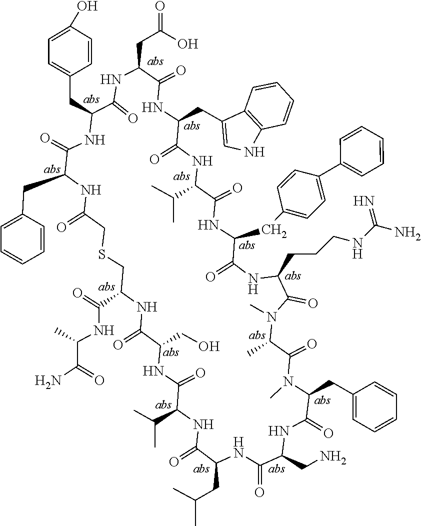 [SMILES: CC(C)C[C@@H]1NC(=O)[C@H](CN)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC1=O]

Example 1271 was prepared on a 50 μmol scale. The yield of the product was 13.1 mg, and its estimated purity by LCMS analysis was 94.9%. Analysis condition A: Retention time=1.82 min; ESI-MS(+) m/z [M+2H]²⁺: 970.1.

### Preparation of Example 1272

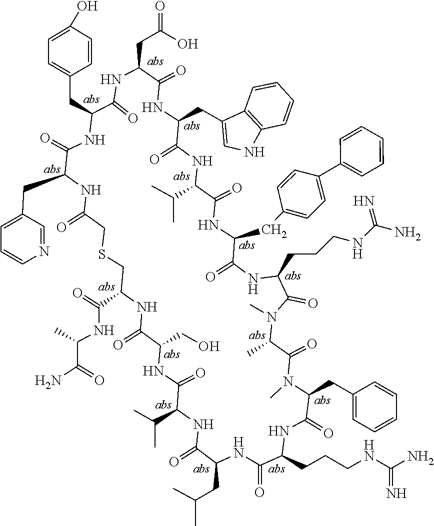 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2cccnc2)NC(=O)CSC[C@@H](C(=O)N[C@@H](C)C(N)=O)NC(=O)[C@H](CO)NC(=O)[C@H](C(C)C)NC1=O]

Example 1272 was prepared on a 50 μmol scale. The yield of the product was 13.9 mg, and its estimated purity by LCMS analysis was 97.2%. Analysis condition A: Retention time=1.88 min; ESI-MS(+) m/z [M+2H]²⁺: 1005.3.

### Preparation of Example 1273

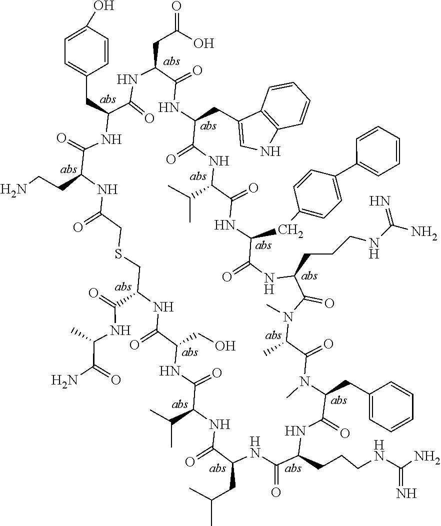 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CCN)NC(=O)CSC[C@@H](C(=O)N[C@@H](C)C(N)=O)NC(=O)[C@H](CO)NC(=O)[C@H](C(C)C)NC1=O]

Example 1273 was prepared on a 50 μmol scale. The yield of the product was 23.1 mg, and its estimated purity by LCMS analysis was 89.3%. Analysis condition A: Retention time=1.81 min: ESI-MS(+) m/z [M+2H]²⁺: 982.1.

### Preparation of Example 1274

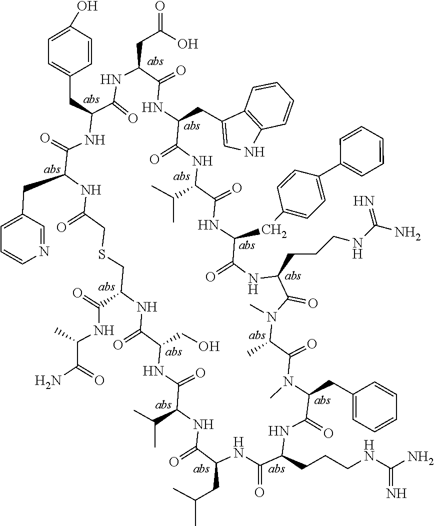 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2cccnc2)NC(=O)CSC[C@@H](C(=O)N[C@@H](C)C(N)=O)NC(=O)[C@H](CO)NC(=O)[C@H](C(C)C)NC1=O]

Example 1274 was prepared on a 50 μmol scale. The yield of the product was 35.5 mg, and its estimated purity by LCMS analysis was 93.8%. Analysis condition B: Retention time=1.52 min: ESI-MS(+) m/z [M+3H]³⁺: 671.1.

### Preparation of Example 1275

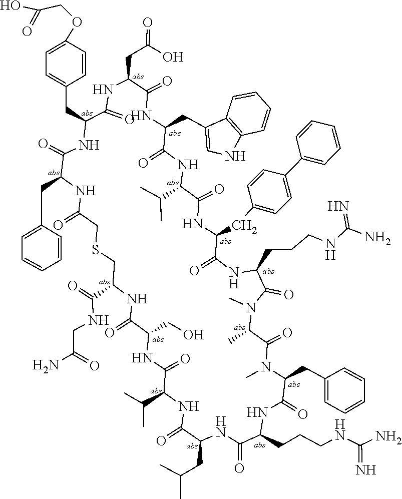 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccccc2)NC(=O)CSC[C@@H](C(=O)NCC(N)=O)NC(=O)[C@H](CO)NC(=O)[C@H](C(C)C)NC1=O]

Example 1275 was prepared on a 50 μmol scale. The yield of the product was 53.3 mg, and its estimated purity by LCMS analysis was 89%. Analysis condition A: Retention time=1.84 min: ESI-MS(+) m/z [M+2H]²⁺: 1034.

### Preparation of Example 1276

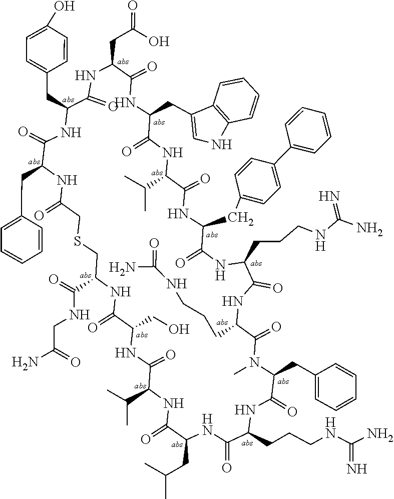 [SMILES: CC(C)C[C@@H]1NC(=O)[C@H](CCCNC(=N)N)NC(=O)[C@H](Cc2ccccc2)N(C)C(=O)[C@H](CCCNC(N)=O)NC(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC1=O]

Example 1276 was prepared on a 50 μmol scale. The yield of the product was 7.5 mg, and its estimated purity by LCMS analysis was 85.5%. Analysis condition B: Retention time=1.66 min: ESI-MS(+) m/z [M+2H]²⁺: 1041.2.

### Preparation of Example 1277

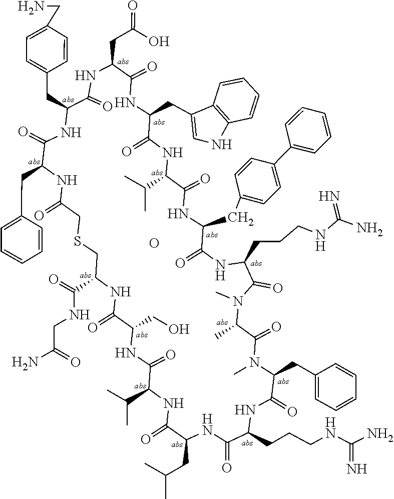

Example 1277 was prepared on a 50 μmol scale. The yield of the product was 5.2 mg, and its estimated purity by LCMS analysis was 97.3%. Analysis condition A: Retention time=1.6 min: ESI-MS(+) m/z [M+2H]²⁺: 1012.3.

### Preparation of Example 1278

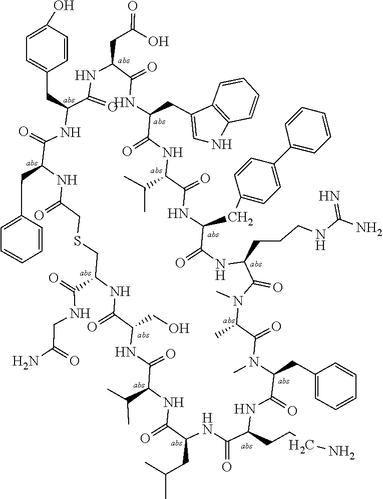 [SMILES: CC(C)C[C@@H]1NC(=O)[C@H](CCCN)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC1=O]

Example 1278 was prepared on a 50 μmol scale. The yield of the product was 8.5 mg, and its estimated purity by LCMS analysis was 97.5%. Analysis condition A: Retention time=1.74 min; ESI-MS(+) m/z [M+2H]²⁺: 984.6.

### Preparation of Example 1279

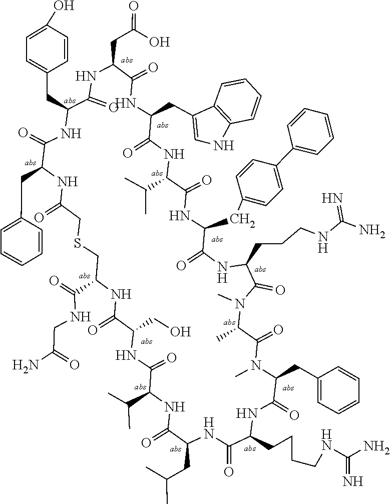 [SMILES: CC(C)C[C@@H]1NC(=O)[C@H](CCCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC1=O]

Example 1279 was prepared on a 50 μmol scale. The yield of the product was 3.3 mg, and its estimated purity by LCMS analysis was 95.2%. Analysis condition A: Retention time=1.77 min; ESI-MS(+) m/z [M+2H]²⁺: 1012.2.

### Preparation of Example 1280

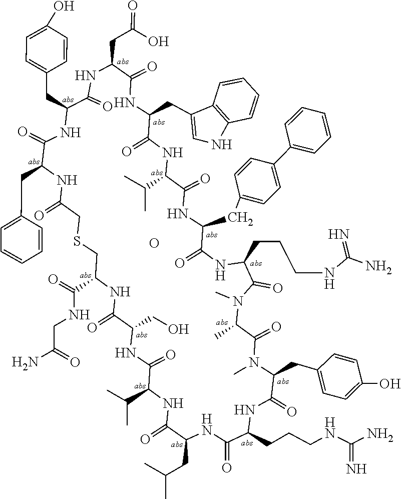

Example 1280 was prepared on a 50 μmol scale. The yield of the product was 16 mg, and its estimated purity by LCMS analysis was 100%. Analysis condition A: Retention time=1.57 min; ESI-MS(+) m/z [M+2H]²⁺: 1014.1.

### Preparation of Example 1281

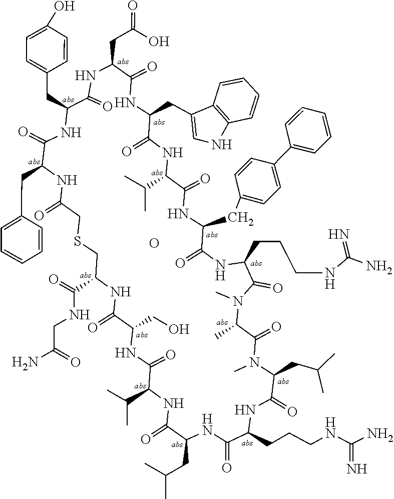

Example 1281 was prepared on a 50 μmol scale. The yield of the product was 17 mg, and its estimated purity by LCMS analysis was 95%. Analysis condition A: Retention time=1.72 min; ESI-MS(+) m/z [M+2H]²⁺: 989.1.

### Preparation of Example 1282

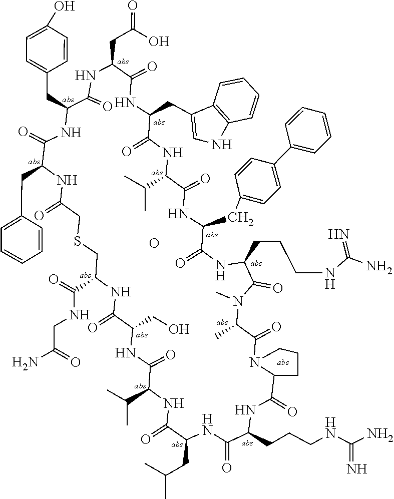

Example 1282 was prepared on a 50 μmol scale. The yield of the product was 18.3 mg, and its estimated purity by LCMS analysis was 97.8%. Analysis condition A: Retention time=1.45 min: ESI-MS(+) m/z [M+2H]²⁺: 973.1.

### Preparation of Example 1283

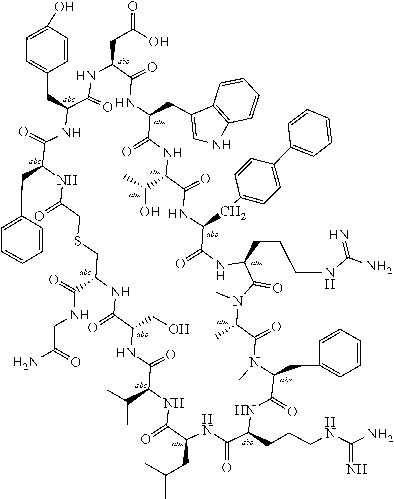 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H]([C@@H](C)O)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC1=O]

Example 1283 was prepared on a 50 μmol scale. The yield of the product was 17.1 mg, and its estimated purity by LCMS analysis was 98.4%. Analysis condition B: Retention time=1.78 min: ESI-MS(+) m/z [M+2H]²⁺: 1006.2.

### Preparation of Example 1284

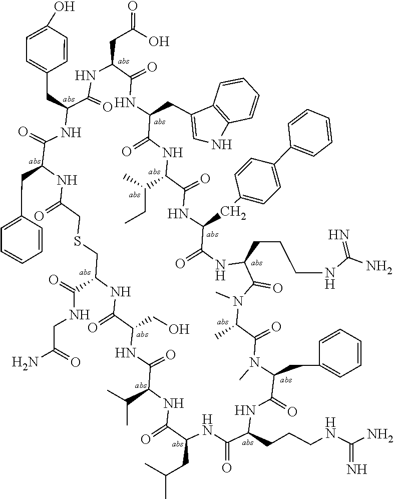 [SMILES: CC[C@H](C)[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC1=O]

Example 1284 was prepared on a 50 μmol scale. The yield of the product was 18.3 mg, and its estimated purity by LCMS analysis was 99.1%. Analysis condition A: Retention time=1.88 min ESI-MS(+) m/z [M+2H]²⁺: 1012.2.

### Preparation of Example 1285

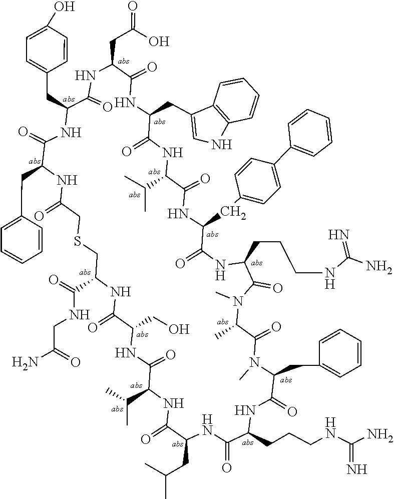

Example 1285 was prepared on a 50 μmol scale. The yield of the product was 19.2 mg, and its estimated purity by LCMS analysis was 94%. Analysis condition A: Retention time=1.85 min: ESI-MS(+) m/z [M+2H]²⁺: 1012.2.

### Preparation of Example 1286

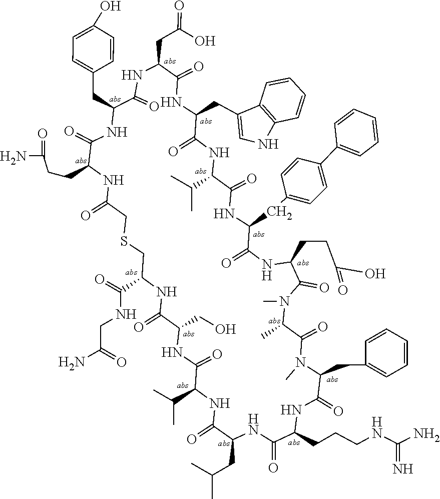 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CCC(N)=O)NC(=O)CSC[C@@H](C(=O)NCC(N)=O)NC(=O)[C@H](CO)NC(=O)[C@H](C(C)C)NC1=O]

Example 1286 was prepared on a 50 μmol scale. The yield of the product was 28.6 mg, and its estimated purity by LCMS analysis was 100%. Analysis condition A: Retention time=1.63 min; ESI-MS(+) m/z [M+2H]²⁺: 1963.3.

### Preparation of Example 1287

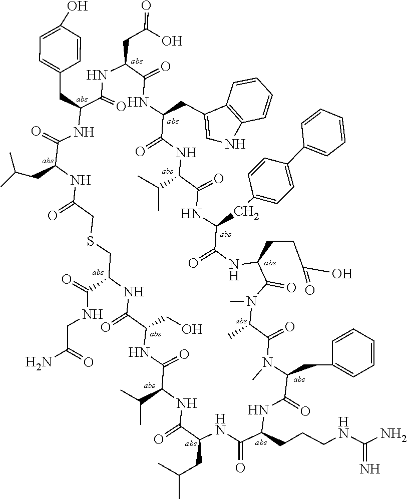 [SMILES: CC(C)C[C@@H]1NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CCCNC(=N)N)NC(=O)[C@H](Cc2ccccc2)N(C)C(=O)[C@H](C)N(C)C(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC1=O]

Example 1287 was prepared on a 50 μmol scale. The yield of the product was 29.7 mg, and its estimated purity by LCMS analysis was 86.6%. Analysis condition B: Retention time=1.85 min; ESI-MS(+) m/z [M+2H]²⁺: 975.2.

### Preparation of Example 1288

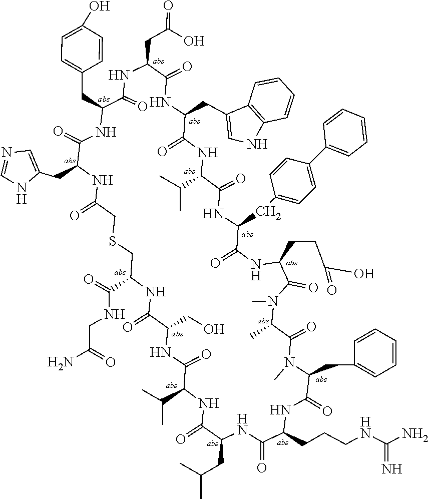 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2cnc[nH]2)NC(=O)CSC[C@@H](C(=O)NCC(N)=O)NC(=O)[C@H](CO)NC(=O)[C@H](C(C)C)NC1=O]

Example 1288 was prepared on a 50 μmol scale. The yield of the product was 27.7 mg, and its estimated purity by LCMS analysis was 97.3%. Analysis condition B: Retention time=1.7 min; ESI-MS(+) m/z [M+2H]²⁺: 986.9.

### Preparation of Example 1289

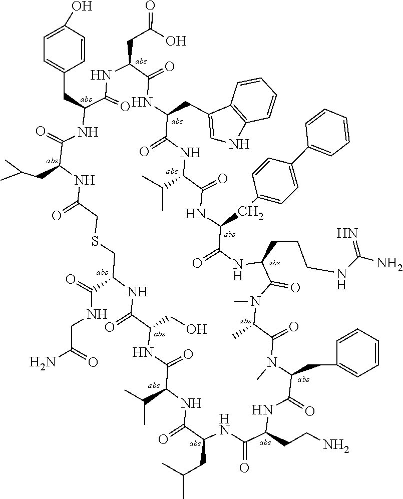 [SMILES: CC(C)C[C@@H]1NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CCN)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC1=O]

Example 1289 was prepared on a 50 μmol scale. The yield of the product was 15.9 mg, and its estimated purity by LCMS analysis was 95.6%. Analysis condition A: Retention time=1.71 min; ESI-MS(+) m/z [M+2H]²⁺: 960.3.

### Preparation of Example 1290

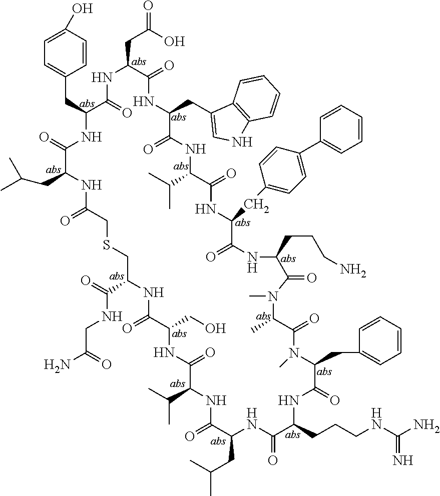 [SMILES: CC(C)C[C@@H]1NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CCCNC(=N)N)NC(=O)[C@H](Cc2ccccc2)N(C)C(=O)[C@H](C)N(C)C(=O)[C@H](CCCN)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC1=O]

Example 1290 was prepared on a 50 μmol scale. The yield of the product was 25.8 mg, and its estimated purity by LCMS analysis was 98.6%. Analysis condition A: Retention time=1.78 min; ESI-MS(+) m/z [M+2H]²⁺: 968.4.

### Preparation of Example 1291

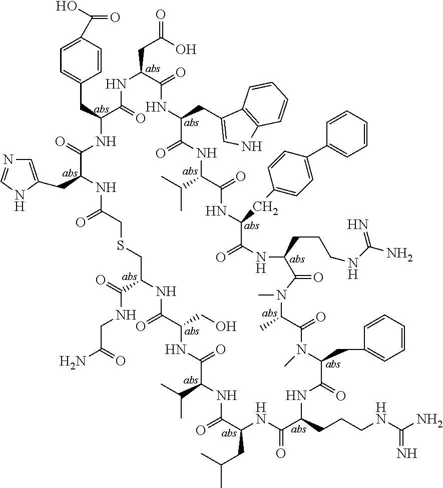 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2cnc[nH]2)NC(=O)CSC[C@@H](C(=O)NCC(N)=O)NC(=O)[C@H](CO)NC(=O)[C@H](C(C)C)NC1=O]

Example 1291 was prepared on a 50 μmol scale. The yield of the product was 6.1 mg, and its estimated purity by LCMS analysis was 98.8%. Analysis condition B: Retention time=1.52 min; ESI-MS(+) m/z [M+2H]²⁺: 1014.4.

### Preparation of Example 1292

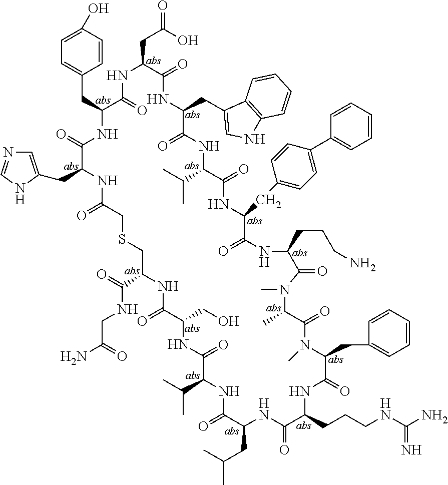 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCN)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2cnc[nH]2)NC(=O)CSC[C@@H](C(=O)NCC(N)=O)NC(=O)[C@H](CO)NC(=O)[C@H](C(C)C)NC1=O]

Example 1292 was prepared on a 50 μmol scale. The yield of the product was 27 mg, and its estimated purity by LCMS analysis was 93.6%. Analysis condition B: Retention time=1.51 min: ESI-MS(+) m/z [M+2H]²⁺: 1956.8.

### Preparation of Example 1293

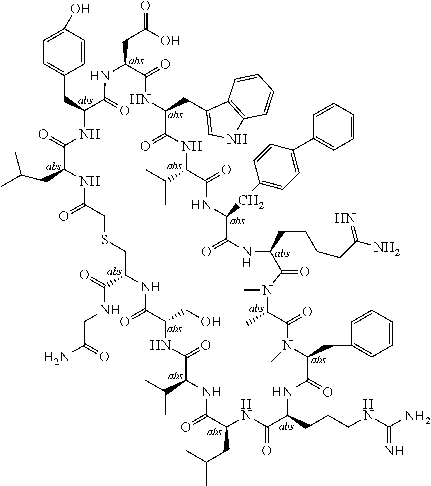 [SMILES: CC(C)C[C@@H]1NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CCCNC(=N)N)NC(=O)[C@H](Cc2ccccc2)N(C)C(=O)[C@H](C)N(C)C(=O)[C@H](CCCCC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC1=O]

Example 1293 was prepared on a 50 μmol scale. The yield of the product was 5 mg, and its estimated purity by LCMS analysis was 98.6%. Analysis condition B: Retention time=1.74 min; ESI-MS(+) m/z [M+2H]²⁺: 974.2.

### Preparation of Example 1294

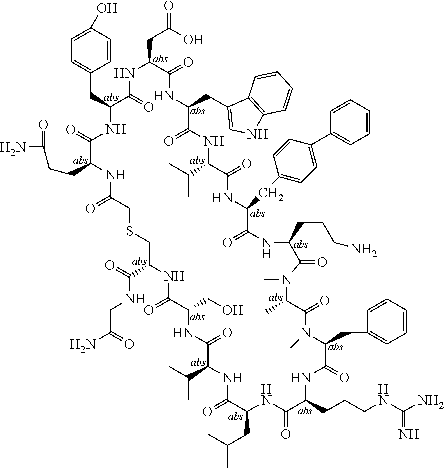 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCN)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CCC(N)=O)NC(=O)CSC[C@@H](C(=O)NCC(N)=O)NC(=O)[C@H](CO)NC(=O)[C@H](C(C)C)NC1=O]

Example 1294 was prepared on a 50 μmol scale. The yield of the product was 32.9 mg, and its estimated purity by LCMS analysis was 95%. Analysis condition A: Retention time=1.78 min; ESI-MS(+) m/z [M+H]⁺: 1947.9.

### Preparation of Example 1295

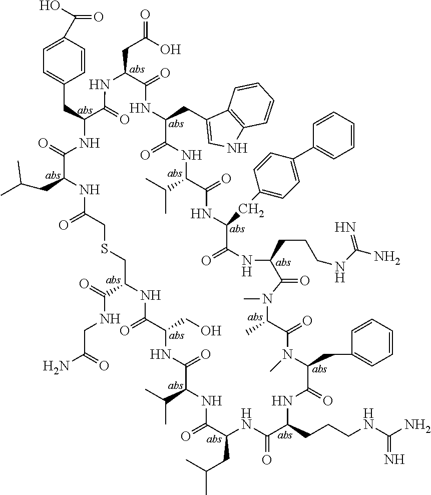 [SMILES: CC(C)C[C@@H]1NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC1=O]

Example 1295 was prepared on a 50 μmol scale. The yield of the product was 12.7 mg, and its estimated purity by LCMS analysis was 97.8%. Analysis condition A: Retention time=1.82 min; ESI-MS(+) m/z [M+2H]²⁺: 1002.

### Preparation of Example 1296

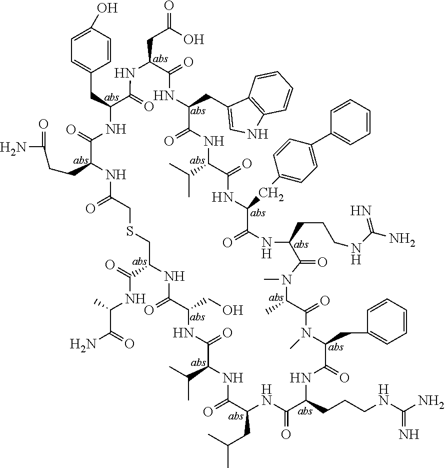 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CCC(N)=O)NC(=O)CSC[C@@H](C(=O)N[C@@H](C)C(N)=O)NC(=O)[C@H](CO)NC(=O)[C@H](C(C)C)NC1=O]

Example 1296 was prepared on a 50 μmol scale. The yield of the product was 6.1 mg, and its estimated purity by LCMS analysis was 95.4%. Analysis condition B: Retention time=1.62 min; ESI-MS(+) m/z [M+2H]²⁺: 1003.3.

### Preparation of Example 1297

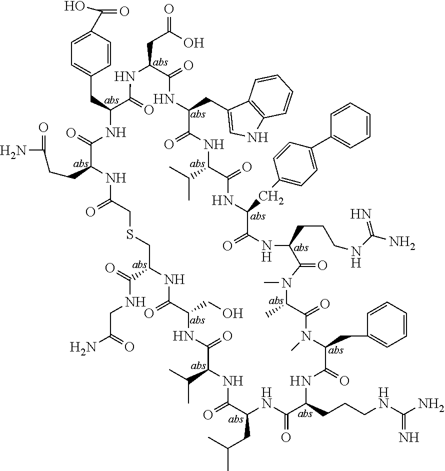 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](CCC(N)=O)NC(=O)CSC[C@@H](C(=O)NCC(N)=O)NC(=O)[C@H](CO)NC(=O)[C@H](C(C)C)NC1=O]

Example 1297 was prepared on a 50 μmol scale. The yield of the product was 22.6 mg, and its estimated purity by LCMS analysis was 100%. Analysis condition A: Retention time=1.73 min; ESI-MS(+) m/z [M+2H]²⁺: 1010.2.

### Preparation of Example 1298

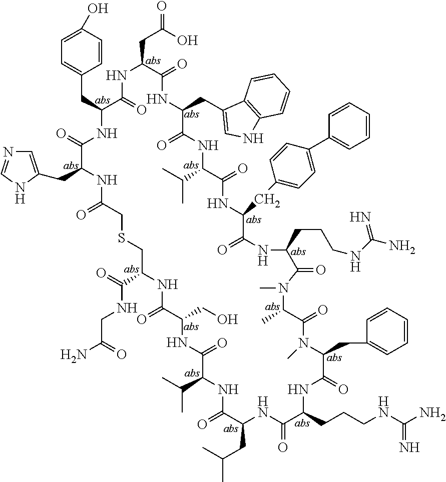 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2cnc[nH]2)NC(=O)CSC[C@@H](C(=O)NCC(N)=O)NC(=O)[C@H](CO)NC(=O)[C@H](C(C)C)NC1=O]

Example 1298 was prepared on a 50 μmol scale. The yield of the product was 8 mg, and its estimated purity by LCMS analysis was 80.7%. Analysis condition A: Retention time=1.61 min: ESI-MS(+) m/z [M+2H]²⁺: 986.5.

### Preparation of Example 1299

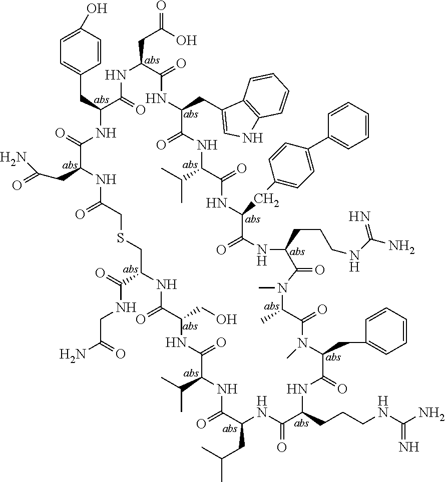 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)NCC(N)=O)NC(=O)[C@H](CO)NC(=O)[C@H](C(C)C)NC1=O]

Example 1299 was prepared on a 50 μmol scale. The yield of the product was 9 mg, and its estimated purity by LCMS analysis was 94.6%. Analysis condition B: Retention time=1.64 min; ESI-MS(+) m/z [M+H]⁺: 1920.2.

### Preparation of Example 1300

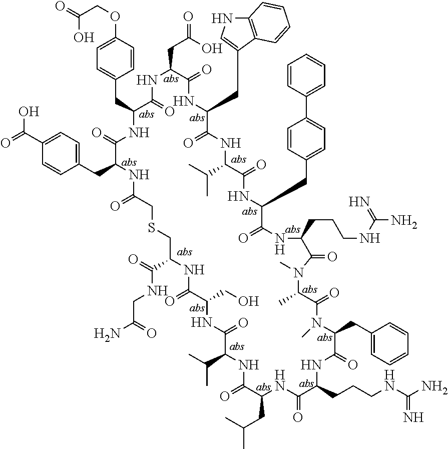 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(C(=O)O)cc2)NC(=O)CSC[C@@H](C(=O)NCC(N)=O)NC(=O)[C@H](CO)NC(=O)[C@H](C(C)C)NC1=O]

Example 1300 was prepared on a 50 μmol scale. The yield of the product was 15.3 mg, and its estimated purity by LCMS analysis was 97.8%. Analysis condition A: Retention time=1.56 min; ESI-MS(+) m/z [M+2H]²⁺: 1056.

### Preparation of Example 1301

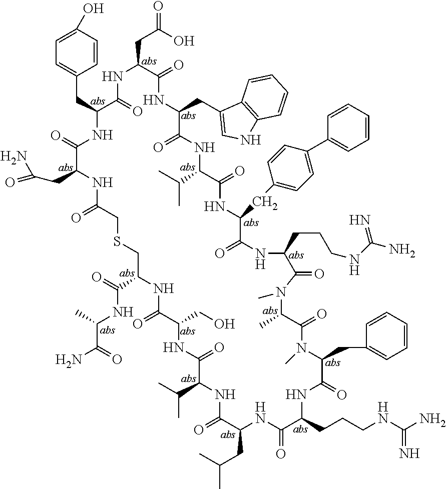 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H](CO)NC(=O)[C@H](C(C)C)NC1=O]

Example 1301 was prepared on a 50 μmol scale. The yield of the product was 7.6 mg, and its estimated purity by LCMS analysis was 93.7%. Analysis condition B: Retention time=1.67 min; ESI-MS(+) m/z [M+H]⁺: 1990.

### Preparation of Example 1302

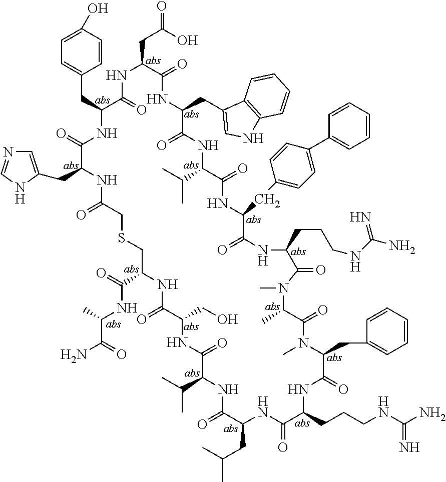 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2cnc[nH]2)NC(=O)CSC[C@@H](C(=O)N[C@@H](C)C(N)=O)NC(=O)[C@H](CO)NC(=O)[C@H](C(C)C)NC1=O]

Example 1302 was prepared on a 50 μmol scale. The yield of the product was 14.6 mg, and its estimated purity by LCMS analysis was 92.8%. Analysis condition A: Retention time=1.63 min; ESI-MS(+) m/z [M+2H]²⁺: 1007.2.

### Preparation of Example 1303

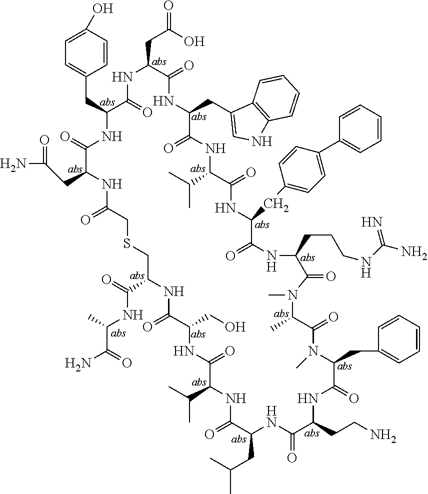 [SMILES: CC(C)C[C@@H]1NC(=O)[C@H](CCN)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H](CO)NC(=O)[C@H](C(C)C)NC1=O]

Example 1303 was prepared on a 50 μmol scale. The yield of the product was 19.7 mg, and its estimated purity by LCMS analysis was 82.4%. Analysis condition B: Retention time=1.6 min; ESI-MS(+) m/z [M+H]⁺: 1934.3.

### Preparation of Example 1304

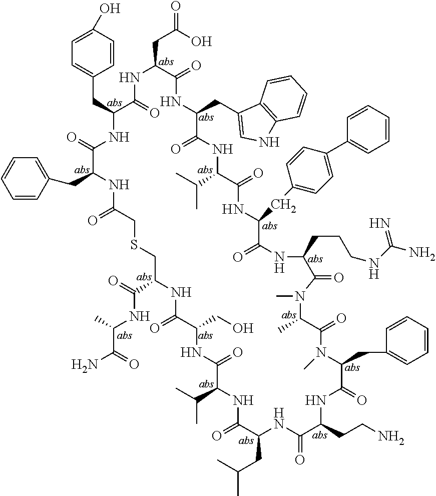 [SMILES: CC(C)C[C@@H]1NC(=O)[C@H](CCN)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC1=O]

Example 1304 was prepared on a 50 μmol scale. The yield of the product was 26.9 mg, and its estimated purity by LCMS analysis was 81.5%. Analysis condition B: Retention time=1.74 min; ESI-MS(+) m/z [M+H]⁺: 1965.2.

### Preparation of Example 1305

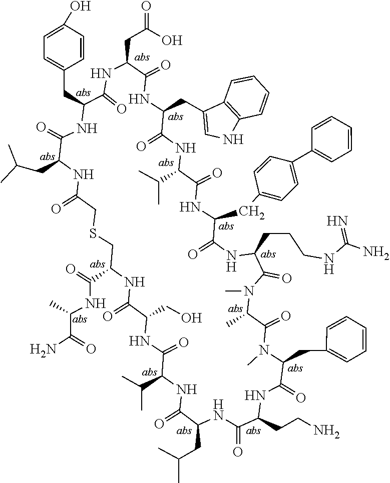 [SMILES: CC(C)C[C@@H]1NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CCN)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC1=O]

Example 1305 was prepared on a 50 μmol scale. The yield of the product was 10.6 mg, and its estimated purity by LCMS analysis was 96.6%. Analysis condition B: Retention time=1.77 min; ESI-MS(+) m/z [M+H]⁺: 1933.1.

### Preparation of Example 1306

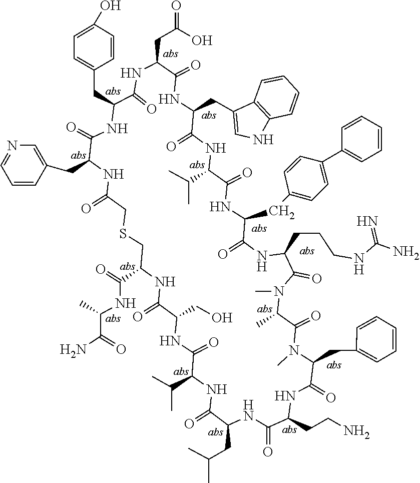 [SMILES: CC(C)C[C@@H]1NC(=O)[C@H](CCN)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2cccnc2)NC(=O)CSC[C@@H](C(=O)N[C@@H](C)C(N)=O)NC(=O)[C@H](CO)NC(=O)[C@H](C(C)C)NC1=O]

Example 1306 was prepared on a 50 μmol scale. The yield of the product was 19.8 mg, and its estimated purity by LCMS analysis was 83%. Analysis condition B: Retention time=1.51 min: ESI-MS(+)m/z [M+H]⁺: 1968.2.

### Preparation of Example 1307

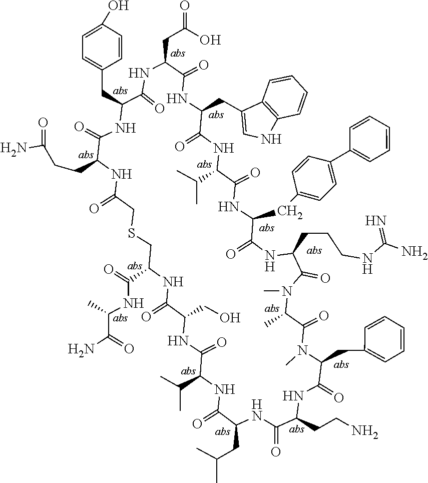 [SMILES: CC(C)C[C@@H]1NC(=O)[C@H](CCN)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CCC(N)=O)NC(=O)CSC[C@@H](C(=O)N[C@@H](C)C(N)=O)NC(=O)[C@H](CO)NC(=O)[C@H](C(C)C)NC1=O]

Example 1307 was prepared on a 50 μmol scale. The yield of the product was 20.5 mg, and its estimated purity by LCMS analysis was 80.4%. Analysis condition B: Retention time=1.57, 1.59 min; ESI-MS(+) m/z [M+2H]²⁺: 974.6, 974.28.

### Preparation of Example 1308

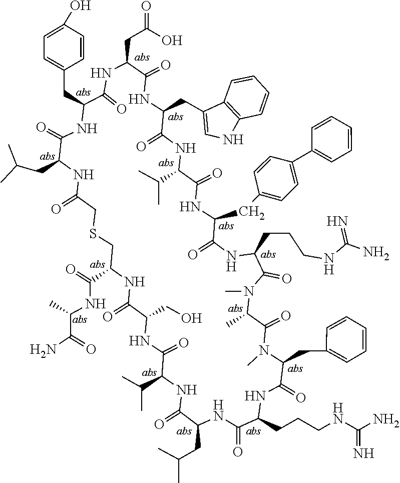 [SMILES: CC(C)C[C@@H]1NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC1=O]

Example 1308 was prepared on a 50 μmol scale. The yield of the product was 6.3 mg, and its estimated purity by LCMS analysis was 96.7%. Analysis condition B: Retention time=1.67 min: ESI-MS(+)m/z [M+3H]³⁺: 664.

### Preparation of Example 1309

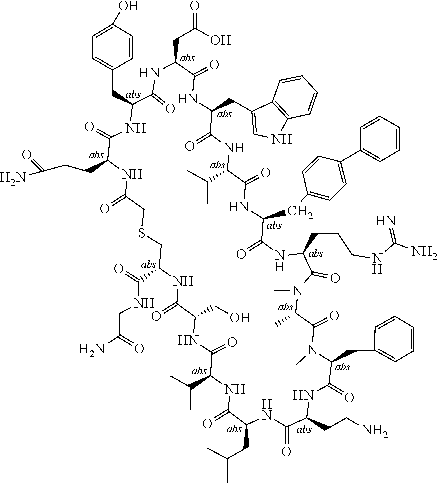 [SMILES: CC(C)C[C@@H]1NC(=O)[C@H](CCN)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CCC(N)=O)NC(=O)CSC[C@@H](C(=O)NCC(N)=O)NC(=O)[C@H](CO)NC(=O)[C@H](C(C)C)NC1=O]

Example 1309 was prepared on a 50 μmol scale. The yield of the product was 6.2 mg, and its estimated purity by LCMS analysis was 86.7%. Analysis condition B: Retention time=1.63 min; ESI-MS(+) m/z [M+H]⁺: 1933.8.

### Preparation of Example 1310

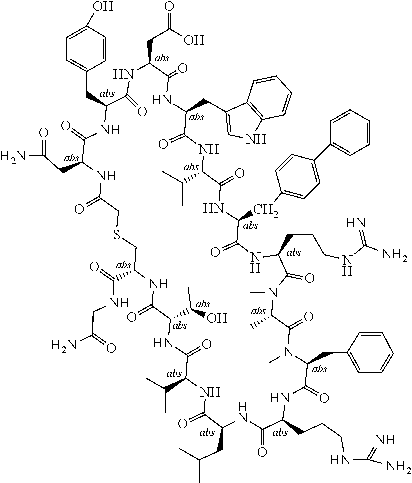 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)NCC(N)=O)NC(=O)[C@H]([C@@H](C)O)NC(=O)[C@H](C(C)C)NC1=O]

Example 1310 was prepared on a 50 μmol scale. The yield of the product was 33.3 mg, and its estimated purity by LCMS analysis was 100%. Analysis condition B: Retention time=1.68 min; ESI-MS(+) m/z [M+2H]²⁺: 996.1.

### Preparation of Example 1311

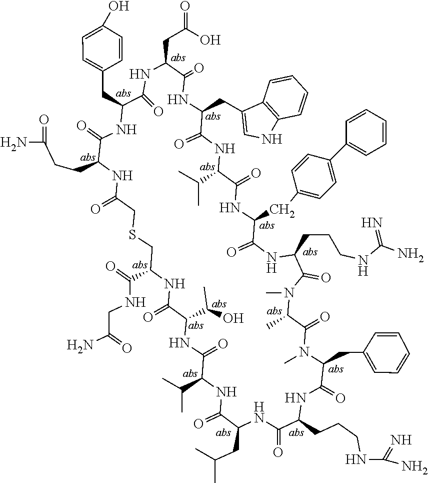 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CCC(N)=O)NC(=O)CSC[C@@H](C(=O)NCC(N)=O)NC(=O)[C@H]([C@@H](C)O)NC(=O)[C@H](C(C)C)NC1=O]

Example 1311 was prepared on a 50 μmol scale. The yield of the product was 34.1 mg, and its estimated purity by LCMS analysis was 98.5%. Analysis condition B: Retention time=1.68 min: ESI-MS(+) m/z [M+2H]²⁺: 1003.2.

### Preparation of Example 1312

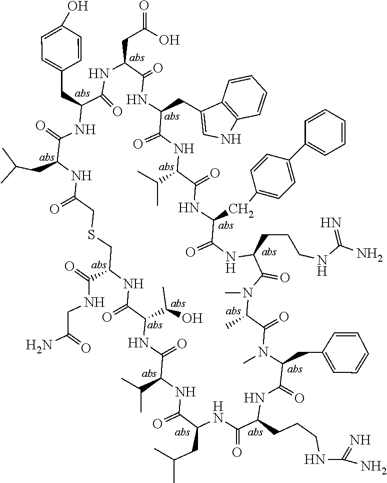 [SMILES: CC(C)C[C@@H]1NC(=O)CSC[C@@H](C(=O)NCC(N)=O)NC(=O)[C@H]([C@@H](C)O)NC(=O)[C@H](C(C)C)NC(=O)[C@H](CC(C)C)NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC1=O]

Example 1312 was prepared on a 50 μmol scale. The yield of the product was 54.9 mg, and its estimated purity by LCMS analysis was 100%. Analysis condition A: Retention time=1.79 min; ESI-MS(+) m/z [M+2H]²⁺: 995.4.

### Preparation of Example 1313

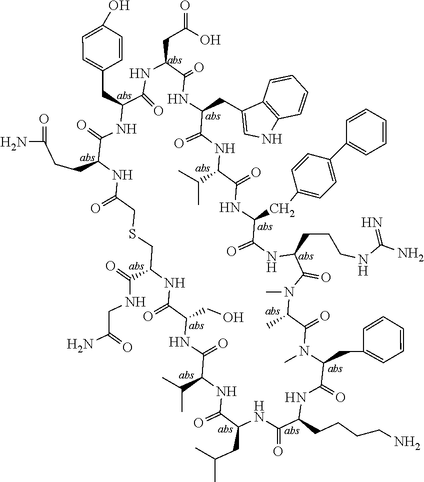 [SMILES: CC(C)C[C@@H]1NC(=O)[C@H](CCCCN)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CCC(N)=O)NC(=O)CSC[C@@H](C(=O)NCC(N)=O)NC(=O)[C@H](CO)NC(=O)[C@H](C(C)C)NC1=O]

Example 1313 was prepared on a 50 μmol scale. The yield of the product was 8.9 mg, and its estimated purity by LCMS analysis was 100%. Analysis condition A: Retention time=1.69 min; ESI-MS(+) m/z [M+H]⁺: 1962.1.

### Preparation of Example 1314

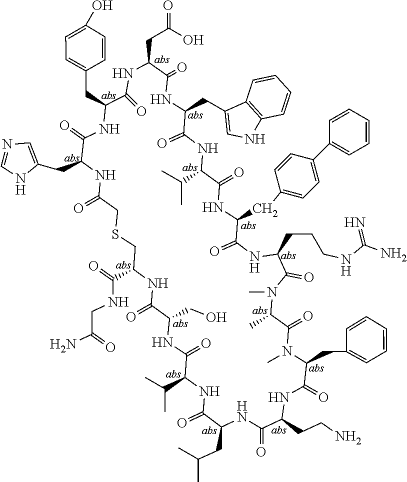 [SMILES: CC(C)C[C@@H]1NC(=O)[C@H](CCN)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2cnc[nH]2)NC(=O)CSC[C@@H](C(=O)NCC(N)=O)NC(=O)[C@H](CO)NC(=O)[C@H](C(C)C)NC1=O]

Example 1314 was prepared on a 50 μmol scale. The yield of the product was 3.8 mg, and its estimated purity by LCMS analysis was 96.9%. Analysis condition A: Retention time=1.64 min: ESI-MS(+) m/z [M+H]⁺: 1943.1.

### Preparation of Example 1315

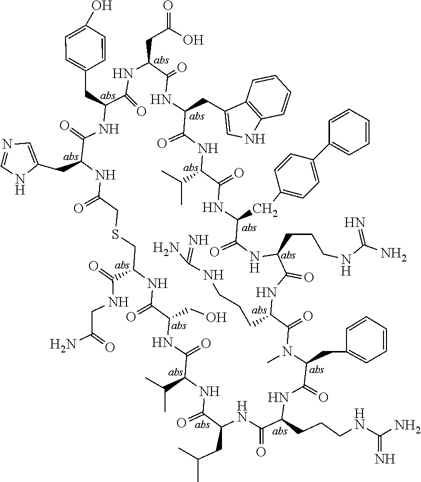 [SMILES: CC(C)C[C@@H]1NC(=O)[C@H](CCCNC(=N)N)NC(=O)[C@H](Cc2ccccc2)N(C)C(=O)[C@H](CCCNC(=N)N)NC(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2cnc[nH]2)NC(=O)CSC[C@@H](C(=O)NCC(N)=O)NC(=O)[C@H](CO)NC(=O)[C@H](C(C)C)NC1=O]

Example 1315 was prepared on a 50 μmol scale. The yield of the product was 8.5 mg, and its estimated purity by LCMS analysis was 90.6%. Analysis condition B: Retention time=1.47 min: ESI-MS(+) m/z [M+2H]²⁺: 1036.

### Preparation of Example 1316

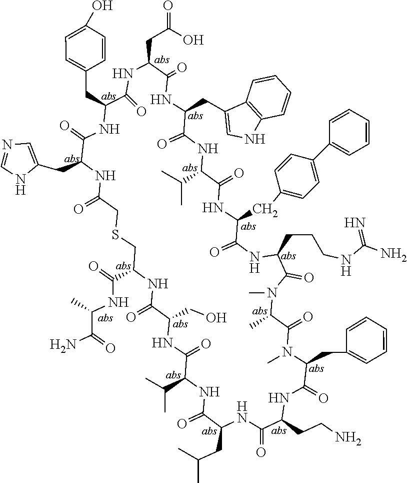 [SMILES: CC(C)C[C@@H]1NC(=O)[C@H](CCN)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2cnc[nH]2)NC(=O)CSC[C@@H](C(=O)N[C@@H](C)C(N)=O)NC(=O)[C@H](CO)NC(=O)[C@H](C(C)C)NC1=O]

Example 1316 was prepared on a 50 μmol scale. The yield of the product was 6 mg, and its estimated purity by LCMS analysis was 92.8%. Analysis condition B: Retention time=1.5 min; ESI-MS(+) m/z [M+2H]²⁺: 979.2.

### Preparation of Example 1317

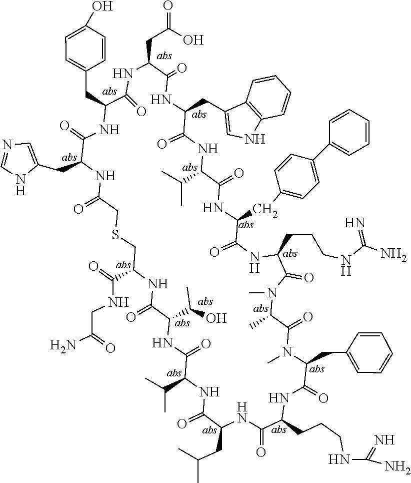 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2cnc[nH]2)NC(=O)CSC[C@@H](C(=O)NCC(N)=O)NC(=O)[C@H]([C@@H](C)O)NC(=O)[C@H](C(C)C)NC1=O]

Example 1317 was prepared on a 50 μmol scale. The yield of the product was 234 mg, and its estimated purity by LCMS analysis was 85.2%. Analysis condition B: Retention time=1.57 min: ESI-MS(+) m/z [M+3H]³⁺: 672.

### Preparation of Example 1318

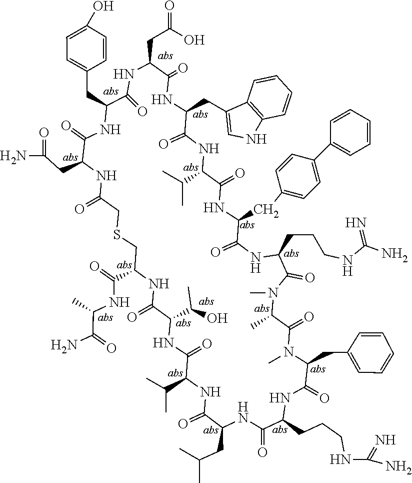 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Example 1318 was prepared on a 50 μmol scale. The yield of the product was 23.6 mg, and its estimated purity by LCMS analysis was 98.3%. Analysis condition B: Retention time=1.7 min: ESI-MS(+) m/z [M+2H]²⁺: 1003.

### Preparation of Example 1319

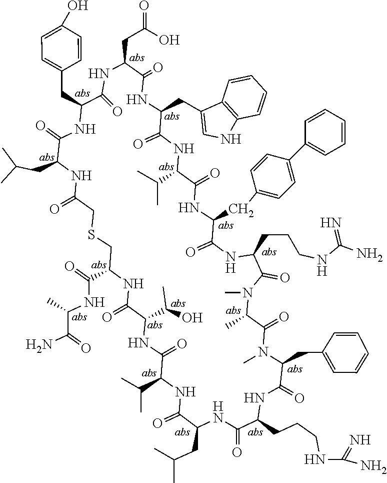 [SMILES: CC(C)C[C@@H]1NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC1=O]

Example 1319 was prepared on a 50 μmol scale. The yield of the product was 19.5 mg, and its estimated purity by LCMS analysis was 95.4%. Analysis condition B: Retention time=1.78 min; ESI-MS(+) m/z [M+2H]²⁺: 1002.5.

### Preparation of Example 1320

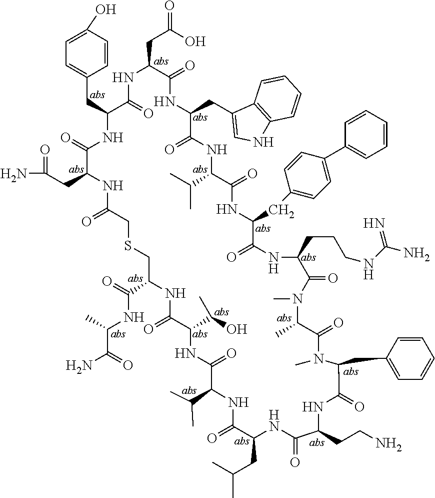 [SMILES: CC(C)C[C@@H]1NC(=O)[C@H](CCN)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Example 1320 was prepared on a 50 μmol scale. The yield of the product was 26.8 mg, and its estimated purity by LCMS analysis was 93.8%. Analysis condition B: Retention time=1.64 min: ESI-MS(+) m/z [M+2H]⁺: 1949.

### Preparation of Example 1321

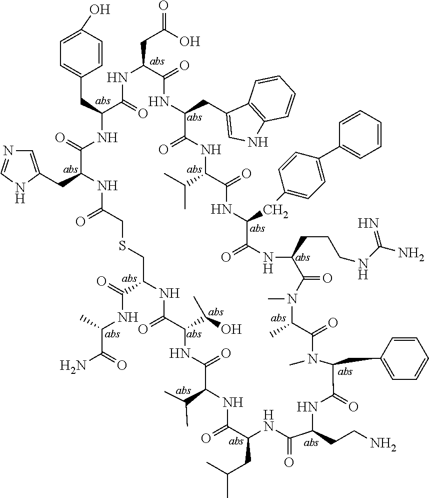 [SMILES: CC(C)C[C@@H]1NC(=O)[C@H](CCN)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2cnc[nH]2)NC(=O)CSC[C@@H](C(=O)N[C@@H](C)C(N)=O)NC(=O)[C@H]([C@@H](C)O)NC(=O)[C@H](C(C)C)NC1=O]

Example 1321 was prepared on a 50 μmol scale. The yield of the product was 15.1 mg, and its estimated purity by LCMS analysis was 95.4%. Analysis condition B: Retention time=1.69 min: ESI-MS(+) m/z [M+H]⁺: 1971.1.

### Preparation of Example 1322

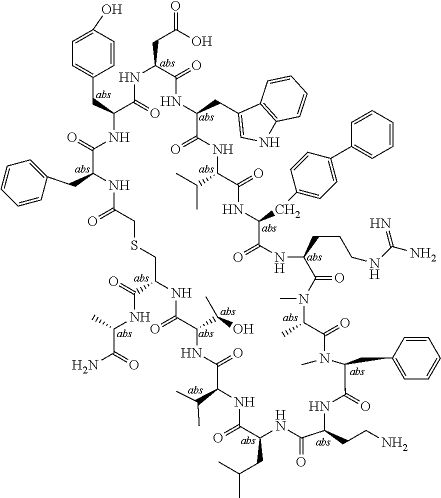 [SMILES: CC(C)C[C@@H]1NC(=O)[C@H](CCN)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC1=O]

Example 1322 was prepared on a 50 μmol scale. The yield of the product was 19.9 mg, and its estimated purity by LCMS analysis was 95.7%. Analysis condition B: Retention time=1.75 min; ESI-MS(+) m/z [M+H]⁺: 1982.4.

### Preparation of Example 1323

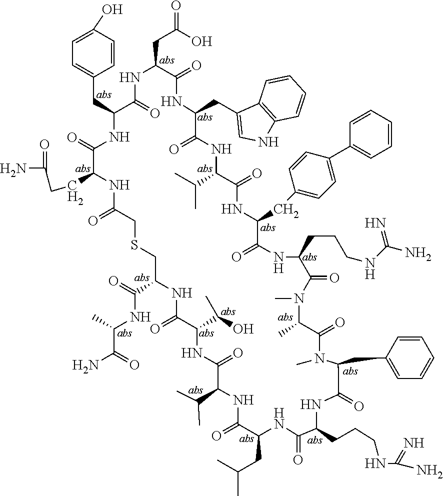 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CCC(N)=O)NC(=O)CSC[C@@H](C(=O)N[C@@H](C)C(N)=O)NC(=O)[C@H]([C@@H](C)O)NC(=O)[C@H](C(C)C)NC1=O]

Example 1323 was prepared on a 50 μmol scale. The yield of the product was 5.1 mg, and its estimated purity by LCMS analysis was 100%. Analysis condition A: Retention time=1.71 min; ESI-MS(+) m/z [M+2H]²⁺: 1010.

### Preparation of Example 1324

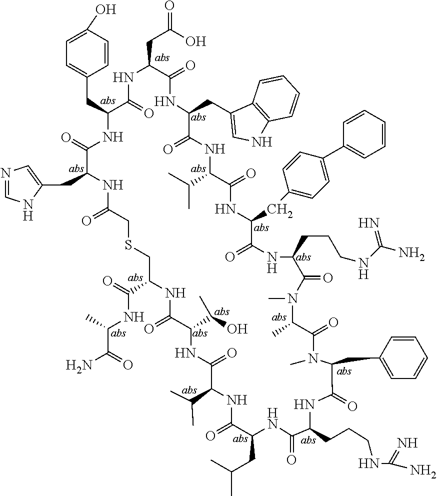 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2cnc[nH]2)NC(=O)CSC[C@@H](C(=O)N[C@@H](C)C(N)=O)NC(=O)[C@H]([C@@H](C)O)NC(=O)[C@H](C(C)C)NC1=O]

Example 1324 was prepared on a 50 μmol scale. The yield of the product was 8.7 mg, and its estimated purity by LCMS analysis was 99.4%. Analysis condition A: Retention time=1.71 min; ESI-MS(+) m/z [M+2H]²⁺: 1014.9.

### Preparation of Example 1325

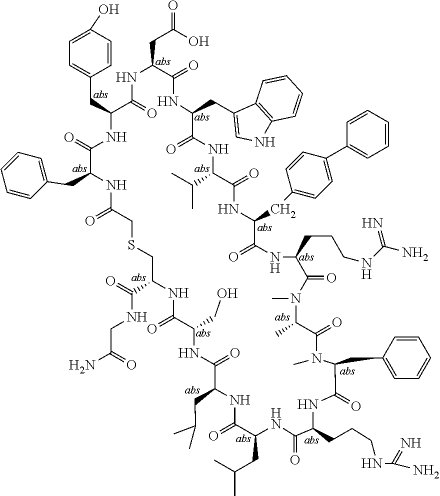 [SMILES: CC(C)C[C@@H]1NC(=O)[C@H](CC(C)C)NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC1=O]

Example 1325 was prepared on a 50 μmol scale. The yield of the product was 26.4 mg, and its estimated purity by LCMS analysis was 94%. Analysis condition A: Retention time=1.98 min: ESI-MS(+) m/z [M+2H]²⁺: 1012.2.

### Preparation of Example 1326

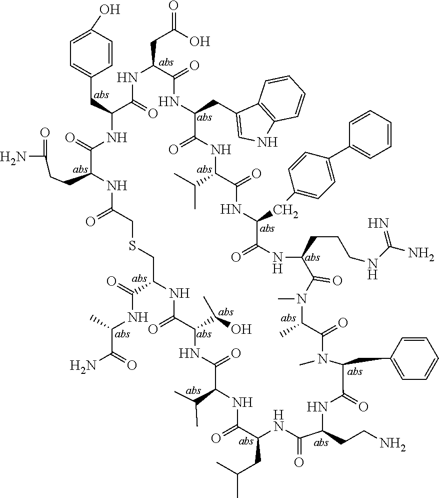 [SMILES: CC(C)C[C@@H]1NC(=O)[C@H](CCN)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CCC(N)=O)NC(=O)CSC[C@@H](C(=O)N[C@@H](C)C(N)=O)NC(=O)[C@H]([C@@H](C)O)NC(=O)[C@H](C(C)C)NC1=O]

Example 1326 was prepared on a 50 μmol scale. The yield of the product was 18.4 mg, and its estimated purity by LCMS analysis was 85%. Analysis condition B: Retention time=1.66 min; ESI-MS(+) m/z [M+H]⁺: 1962.

### Preparation of Example 1327

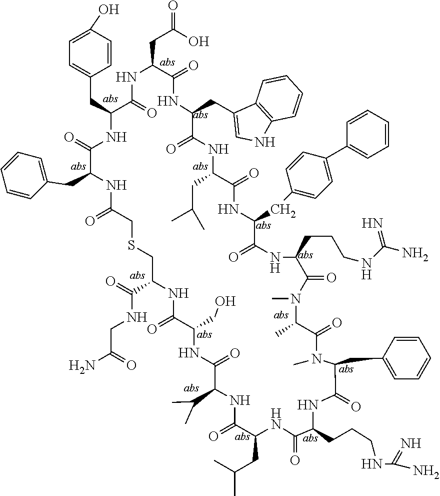 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC1=O]

Example 1327 was prepared on a 50 μmol scale. The yield of the product was 37.2 mg, and its estimated purity by LCMS analysis was 92.2%. Analysis condition A: Retention time=1.9 min: ESI-MS(+) m/z [M+2H]²⁺: 1012.2.

### Preparation of Example 1328

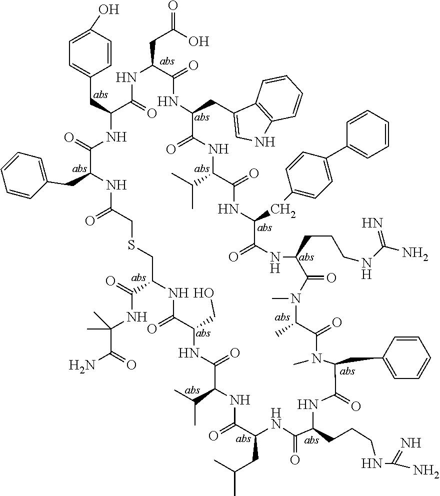 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NC(C)(C)C(N)=O)NC(=O)[C@H](CO)NC(=O)[C@H](C(C)C)NC1=O]

Example 1328 was prepared on a 50 μmol scale. The yield of the product was 30.2 mg, and its estimated purity by LCMS analysis was 100%. Analysis condition B: Retention time=1.72 min: ESI-MS(+) m/z [M+3H]³⁺: 680.

### Preparation of Example 1329

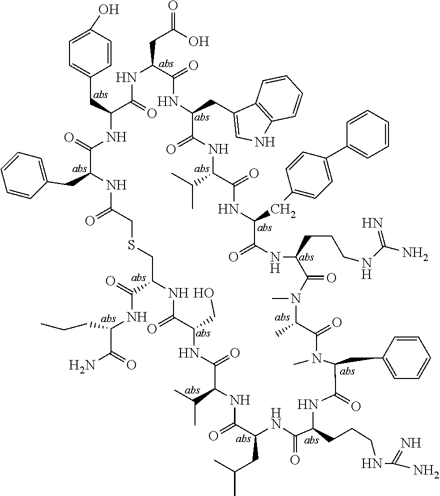 [SMILES: CCC[C@H](NC(=O)[C@@H]1CSCC(=O)N[C@@H](Cc2ccccc2)C(=O)N[C@@H](Cc2ccc(O)cc2)C(=O)N[C@@H](CC(=O)O)C(=O)N[C@@H](Cc2c[nH]c3ccccc23)C(=O)N[C@@H](C(C)C)C(=O)N[C@@H](Cc2ccc(-c3ccccc3)cc2)C(=O)N[C@@H](CCCNC(=N)N)C(=O)N(C)[C@@H](C)C(=O)N(C)[C@@H](Cc2ccccc2)C(=O)N[C@@H](CCCNC(=N)N)C(=O)N[C@@H](CC(C)C)C(=O)N[C@@H](C(C)C)C(=O)N[C@@H](CO)C(=O)N1)C(N)=O]

Example 1329 was prepared on a 50 μmol scale. The yield of the product was 8.4 mg, and its estimated purity by LCMS analysis was 100%. Analysis condition A: Retention time=1.93 min; ESI-MS(+) m/z [M+2H]²⁺: 1026.2.

### Preparation of Example 1330

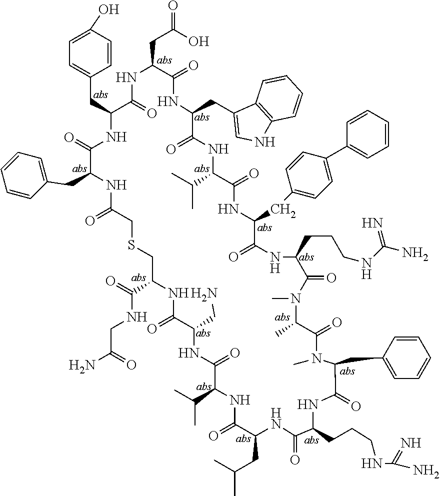 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CN)NC(=O)[C@H](C(C)C)NC1=O]

Example 1330 was prepared on a 50 μmol scale. The yield of the product was 37.1 mg, and its estimated purity by LCMS analysis was 98.6%. Analysis condition B: Retention time=1.7 min: ESI-MS(+) m/z [M+2H]²⁺: 1005.2.

### Preparation of Example 1331

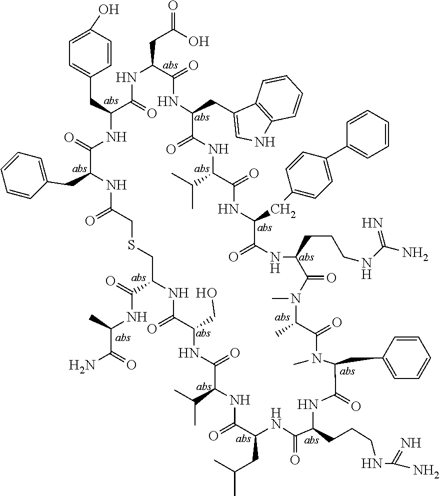 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@H](C)C(N)=O)NC(=O)[C@H](CO)NC(=O)[C@H](C(C)C)NC1=O]

Example 1331 was prepared on a 50 μmol scale. The yield of the product was 32.3 mg, and its estimated purity by LCMS analysis was 96.2%. Analysis condition B: Retention time=1.81 min; ESI-MS(+) m/z [M+2H]²⁺: 1012.2.

### Preparation of Example 1332

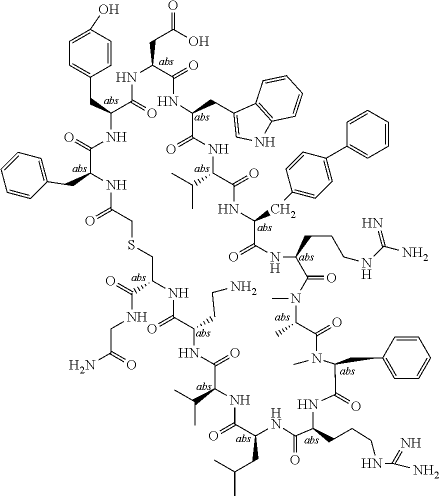 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CCN)NC(=O)[C@H](C(C)C)NC1=O]

Example 1332 was prepared on a 50 μmol scale. The yield of the product was 7.2 mg, and its estimated purity by LCMS analysis was 90.7%. Analysis condition B: Retention time=1.7 min; ESI-MS(+) m/z [M+2H]²⁺: 1012.

### Preparation of Example 1333

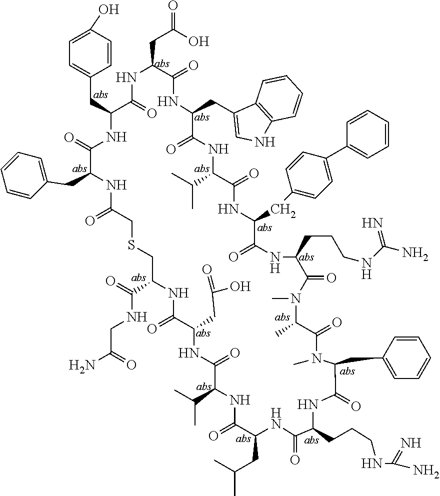 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CC(=O)O)NC(=O)[C@H](C(C)C)NC1=O]

Example 1333 was prepared on a 50 μmol scale. The yield of the product was 49 mg, and its estimated purity by LCMS analysis was 100%. Analysis condition B: Retention time=1.74 min; ESI-MS(+) m/z [M+2H]²⁺: 1019.2.

### Preparation of Example 1334

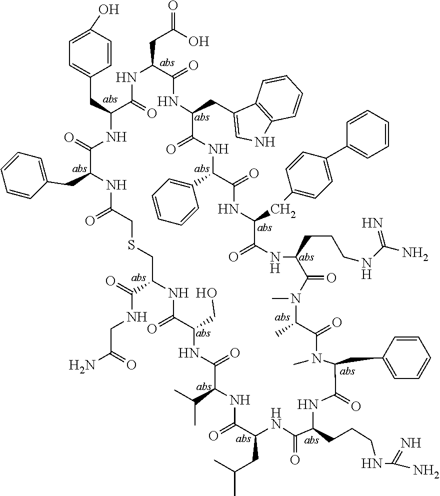 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](c2ccccc2)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC1=O]

Example 1334 was prepared on a 50 μmol scale. The yield of the product was 14.8 mg, and its estimated purity by LCMS analysis was 100%. Analysis condition A: Retention time=1.86 min; ESI-MS(+) m/z [M+2H]²⁺: 1022.

### Preparation of Example 1335

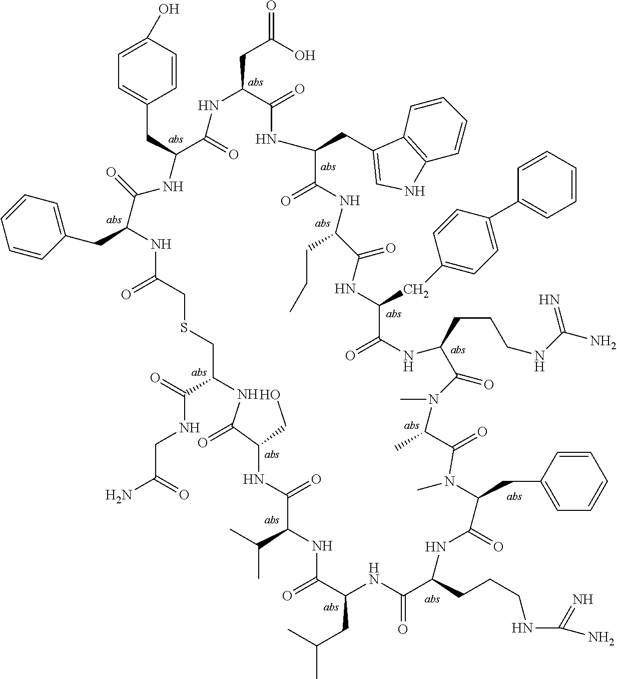 [SMILES: CCC[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC1=O]

Example 1335 was prepared on a 50 μmol scale. The yield of the product was 28 mg, and its estimated purity by LCMS analysis was 93.2%. Analysis condition A: Retention time=1.85 min: ESI-MS(+) m/z [M+2H]²⁺: 1005.2.

### Preparation of Example 1336

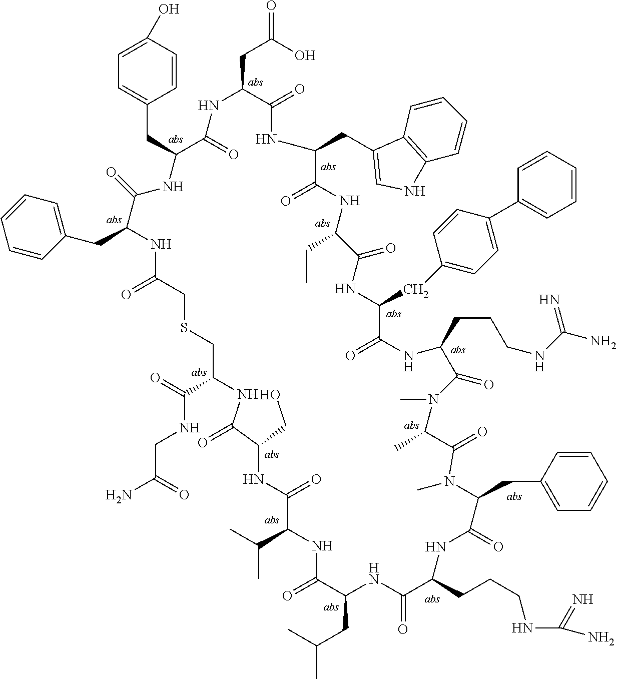 [SMILES: CC[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC1=O]

Example 1336 was prepared on a 50 μmol scale. The yield of the product was 12.6 mg, and its estimated purity by LCMS analysis was 92.6%. Analysis condition B: Retention time=1.69 min; ESI-MS(+) m/z [M+2H]²⁺: 998.1.

### Preparation of Example 1337

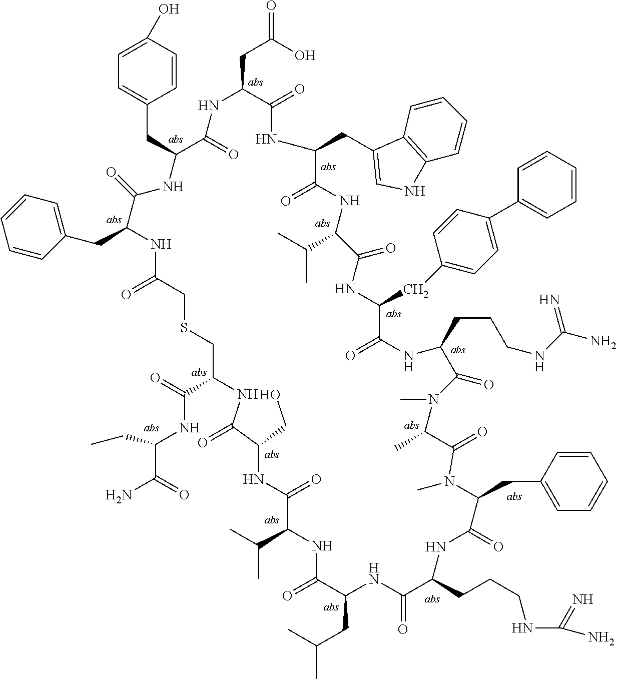 [SMILES: CC[C@H](NC(=O)[C@@H]1CSCC(=O)N[C@@H](Cc2ccccc2)C(=O)N[C@@H](Cc2ccc(O)cc2)C(=O)N[C@@H](CC(=O)O)C(=O)N[C@@H](Cc2c[nH]c3ccccc23)C(=O)N[C@@H](C(C)C)C(=O)N[C@@H](Cc2ccc(-c3ccccc3)cc2)C(=O)N[C@@H](CCCNC(=N)N)C(=O)N(C)[C@@H](C)C(=O)N(C)[C@@H](Cc2ccccc2)C(=O)N[C@@H](CCCNC(=N)N)C(=O)N[C@@H](CC(C)C)C(=O)N[C@@H](C(C)C)C(=O)N[C@@H](CO)C(=O)N1)C(N)=O]

Example 1337 was prepared on a 50 μmol scale. The yield of the product was 2.8 mg, and its estimated purity by LCMS analysis was 88.7%. Analysis condition B: Retention time=1.8 min: ESI-MS(+) m/z [M+2H]²⁺: 1020.1.

### Preparation of Example 1338

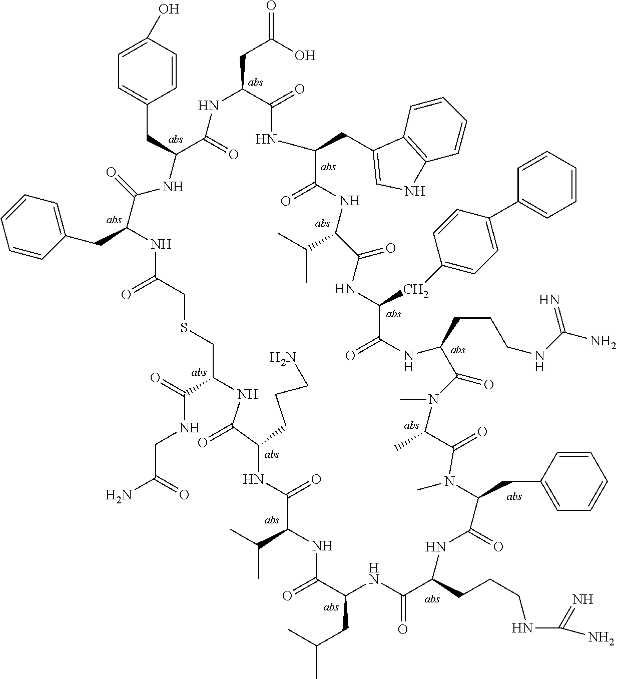 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CCCN)NC(=O)[C@H](C(C)C)NC1=O]

Example 1338 was prepared on a 50 μmol scale. The yield of the product was 39.5 mg, and its estimated purity by LCMS analysis was 83.8%. Analysis condition B: Retention time=1.64 min; ESI-MS(+) m/z [M+2H]²⁺: 1019.1.

### Preparation of Example 1339

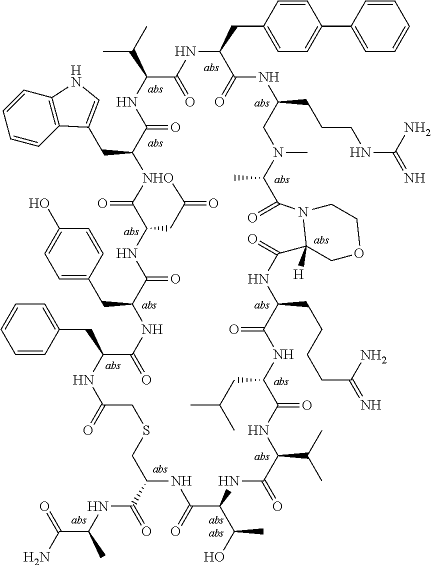

Example 1339 was prepared on a 50 μmol scale. The yield of the product was 3 mg, and its estimated purity by LCMS analysis was 89.5%. Analysis condition B: Retention time=1.59 min; ESI-MS(+) m/z [M+2H]²⁺: 995.1.

### Preparation of Example 1340

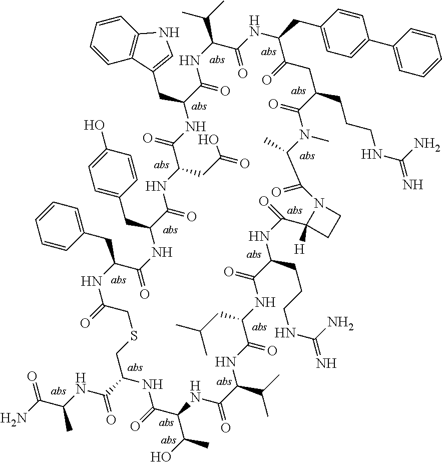 [SMILES: CC(C)C[C@@H]1NC(=O)[C@H](CCCNC(=N)N)NC(=O)[C@H]2CCN2C(=O)[C@H](C)N(C)C(=O)[C@H](CCCNC(=N)N)CC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC1=O]

Example 1340 was prepared on a 50 μmol scale. The yield of the product was 6.2 mg, and its estimated purity by LCMS analysis was 96.6%. Analysis condition A: Retention time=1.42 min; ESI-MS(+) m/z [M+2H]²⁺: 980.3.

### Preparation of Example 1341

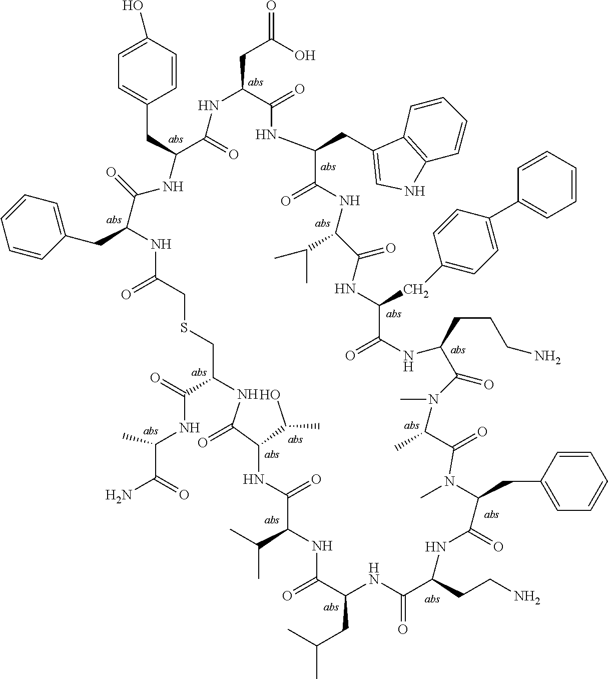 [SMILES: CC(C)C[C@@H]1NC(=O)[C@H](CCN)NC(=O)[C@H](Cc2ccccc2)N(C)C(=O)[C@H](C)N(C)C(=O)[C@H](CCCN)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC1=O]

Example 1341 was prepared on a 50 μmol scale. The yield of the product was 23.7 mg, and its estimated purity by LCMS analysis was 92.8%. Analysis condition B: Retention time=1.79 min: ESI-MS(+) m/z [M+H]⁺: 1940.1.

### Preparation of Example 1342

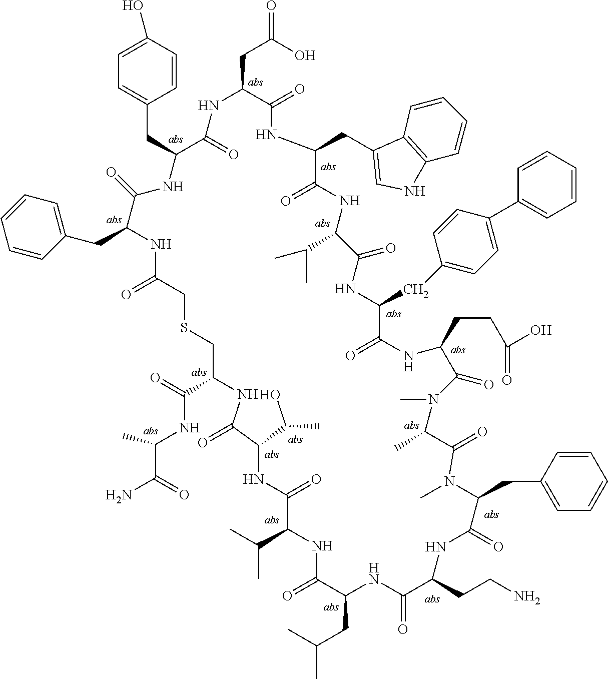 [SMILES: CC(C)C[C@@H]1NC(=O)[C@H](CCN)NC(=O)[C@H](Cc2ccccc2)N(C)C(=O)[C@H](C)N(C)C(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC1=O]

Example 1342 was prepared on a 50 μmol scale. The yield of the product was 30.8 mg, and its estimated purity by LCMS analysis was 91.2%. Analysis condition B: Retention time=1.89 min; ESI-MS(+) m/z [M+H]⁺: 1955.

### Preparation of Example 1343

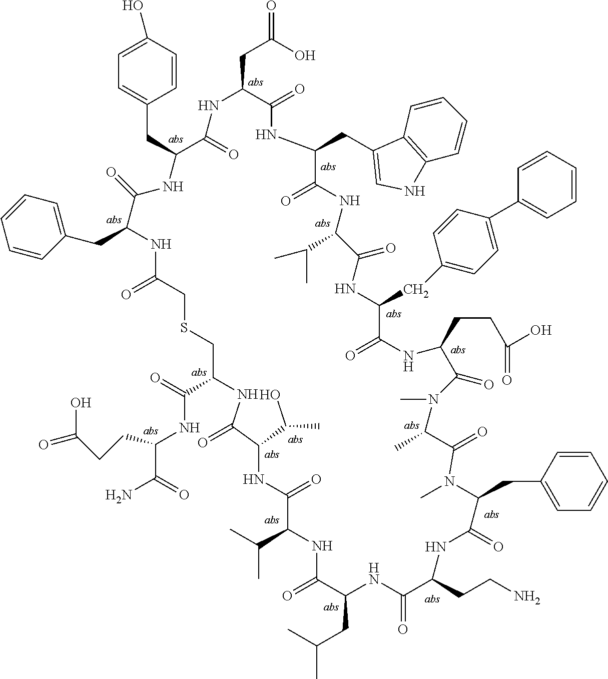 [SMILES: CC(C)C[C@@H]1NC(=O)[C@H](CCN)NC(=O)[C@H](Cc2ccccc2)N(C)C(=O)[C@H](C)N(C)C(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](CCC(=O)O)C(N)=O)NC(=O)[C@H]([C@@H](C)O)NC(=O)[C@H](C(C)C)NC1=O]

Example 1343 was prepared on a 100 μmol scale. The yield of the product was 49 mg, and its estimated purity by LCMS analysis was 92.4%. Analysis condition A: Retention time=1.55 min; ESI-MS(+) m/z [M+2H]²⁺: 1007.

### Preparation of Example 1344

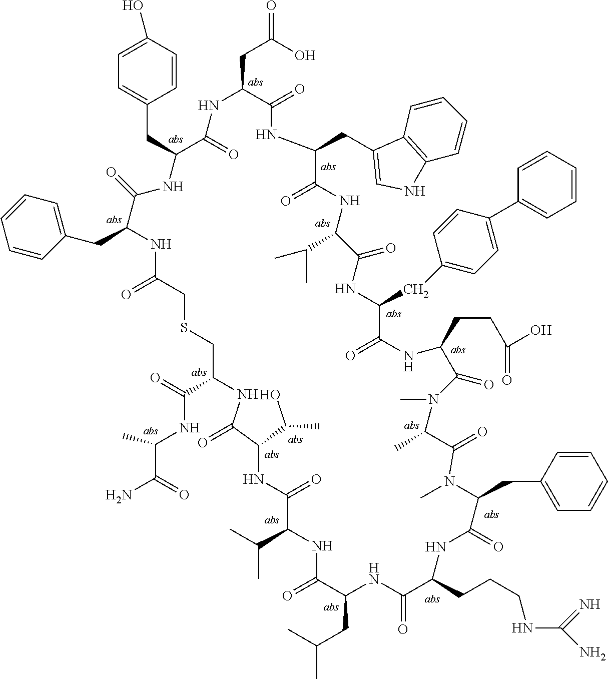 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC1=O]

Example 1344 was prepared on a 50 μmol scale. The yield of the product was 13.2 mg, and its estimated purity by LCMS analysis was 92.2%. Analysis condition A: Retention time=1.72 min; ESI-MS(+) m/z [M+2H]²⁺: 1005.9.

### Preparation of Example 1345

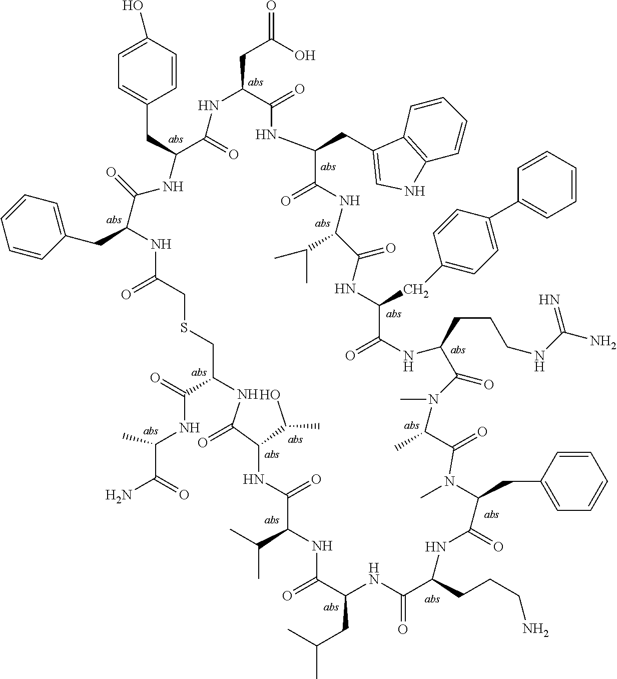 [SMILES: CC(C)C[C@@H]1NC(=O)[C@H](CCCN)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC1=O]

Example 1345 was prepared on a 50 μmol scale. The yield of the product was 9.9 mg, and its estimated purity by LCMS analysis was 100%. Analysis condition B: Retention time=1.72 min; ESI-MS(+) m/z [M+2H]²⁺: 998.5.

### Preparation of Example 1346

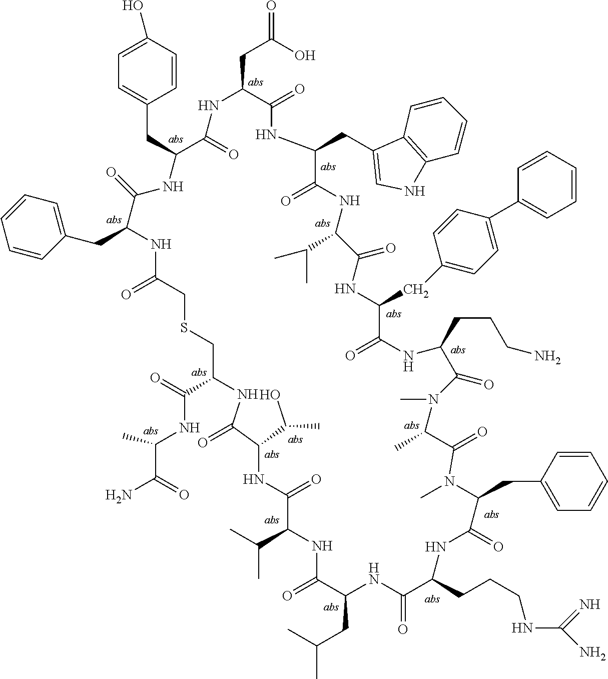 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCN)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC1=O]

Example 1346 was prepared on a 50 μmol scale. The yield of the product was 5.3 mg, and its estimated purity by LCMS analysis was 90.4%. Analysis condition B: Retention time=1.81 min; ESI-MS(+) m/z [M+H]⁺: 1995.2.

### Preparation of Example 1347

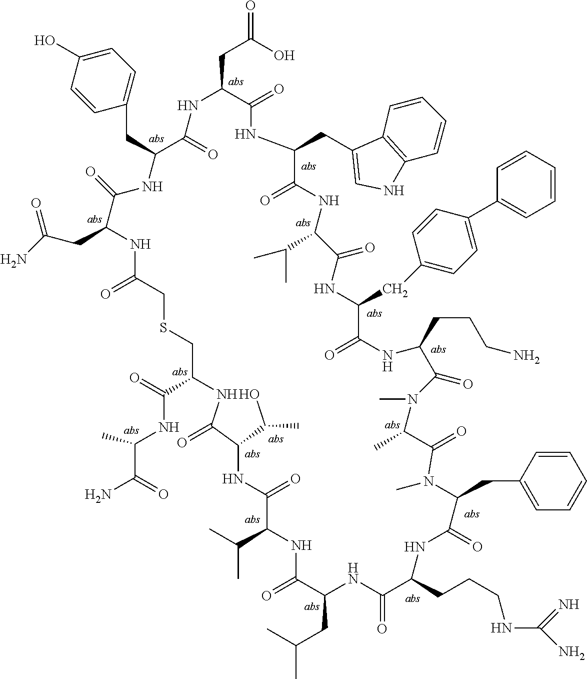 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCN)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Example 1347 was prepared on a 50 μmol scale. The yield of the product was 13.9 mg, and its estimated purity by LCMS analysis was 90.3%. Analysis condition B: Retention time=1.57 min; ESI-MS(+) m/z [M+2H]²⁺: 981.9.

### Preparation of Example 1348

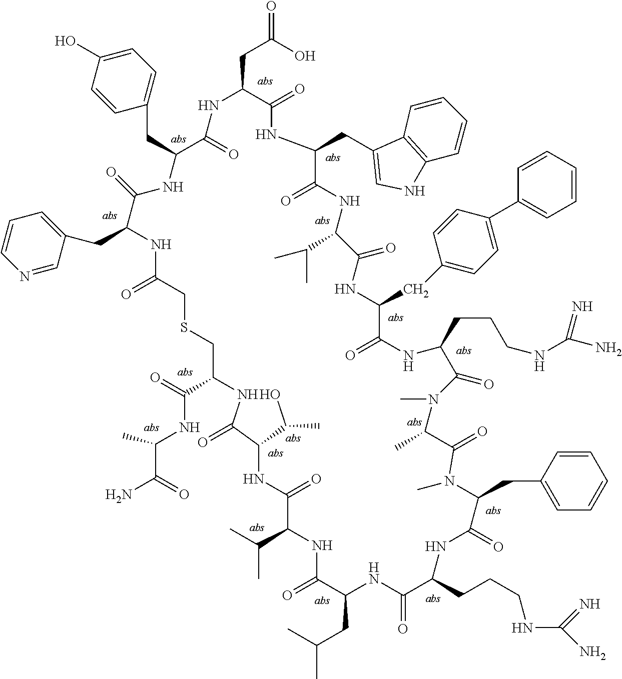 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2cccnc2)NC(=O)CSC[C@@H](C(=O)N[C@@H](C)C(N)=O)NC(=O)[C@H]([C@@H](C)O)NC(=O)[C@H](C(C)C)NC1=O]

Example 1348 was prepared on a 50 μmol scale. The yield of the product was 20 mg, and its estimated purity by LCMS analysis was 94.1%. Analysis condition A: Retention time=1.79 min; ESI-MS(+) m/z [M+2H]²⁺: 1020.1.

### Preparation of Example 1349

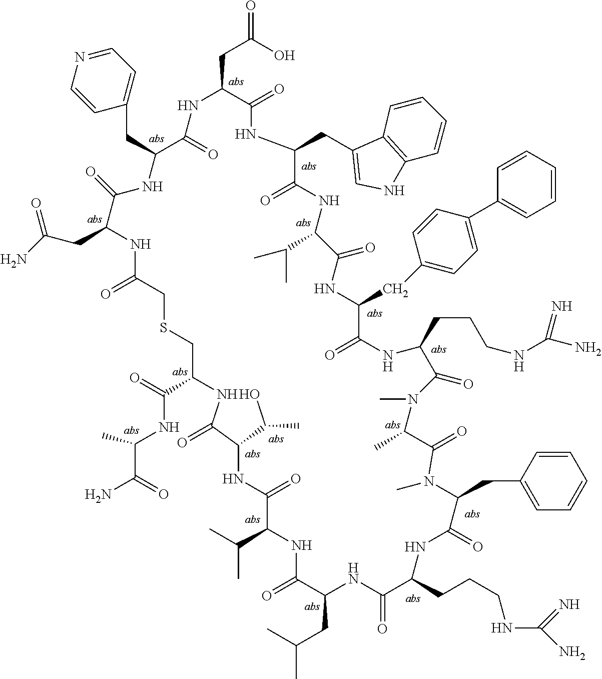 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Example 1349 was prepared on a 50 μmol scale. The yield of the product was 15.2 mg, and its estimated purity by LCMS analysis was 100%. Analysis condition A: Retention time=1.74 min: ESI-MS(+) m/z [M+H]⁺: 1989.3.

### Preparation of Example 1350

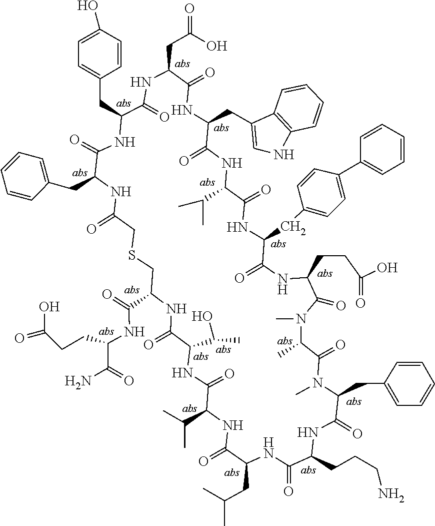

Example 1350 was prepared on a 50 μmol scale. The yield of the product was 15 mg, and its estimated purity by LCMS analysis was 92.1%. Analysis condition A: Retention time=1.6, 1.63 min; ESI-MS(+) m/z [M+2H]²⁺: 1013.97, 1013.97.

### Preparation of Example 1351

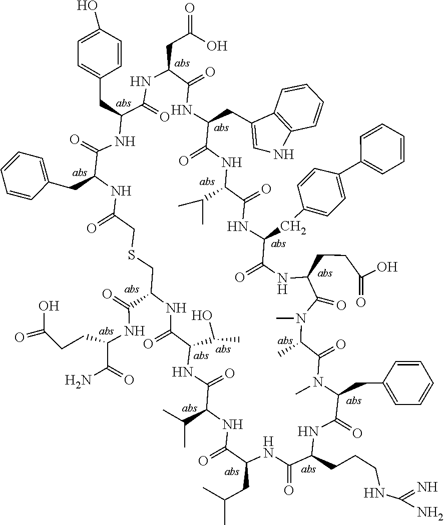

Example 1351 was prepared on a 100 μmol scale. The yield of the product was 21.9 mg, and its estimated purity by LCMS analysis was 97.3%. Analysis condition A: Retention time=1.61 min; ESI-MS(+) m/z [M+2H]²⁺: 1035.2.

### Preparation of Example 1352

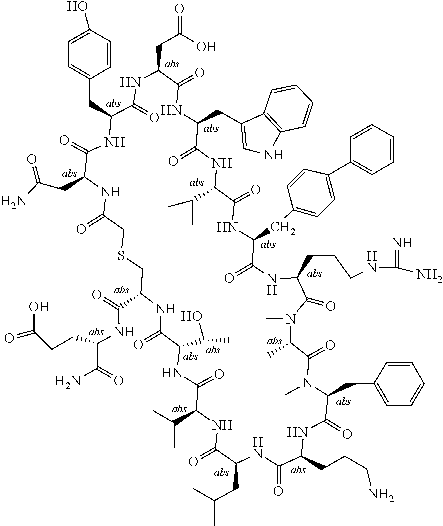

Example 1352 was prepared on a 100 μmol scale. The yield of the product was 10.7 mg, and its estimated purity by LCMS analysis was 97.8%. Analysis condition A: Retention time=1.64 min; ESI-MS(+) m/z [M+2H]²⁺: 1011.2.

### Preparation of Example 1353

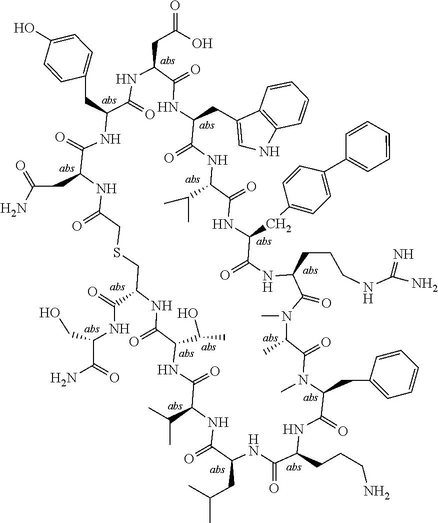

Example 1353 was prepared on a 50 μmol scale. The yield of the product was 11.2 mg, and its estimated purity by LCMS analysis was 96.5%. Analysis condition A: Retention time=1.68 min; ESI-MS(+) m/z [M+H]⁺: 1978.

### Preparation of Example 1354

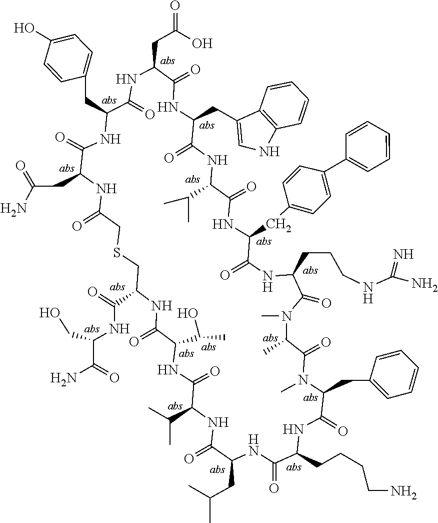

Example 1354 was prepared on a 50 μmol scale. The yield of the product was 5.6 mg, and its estimated purity by LCMS analysis was 98.4%. Analysis condition B: Retention time=1.69 min: ESI-MS(+) m/z [M+2H]²⁺: 997.3.

### Preparation of Example 1355

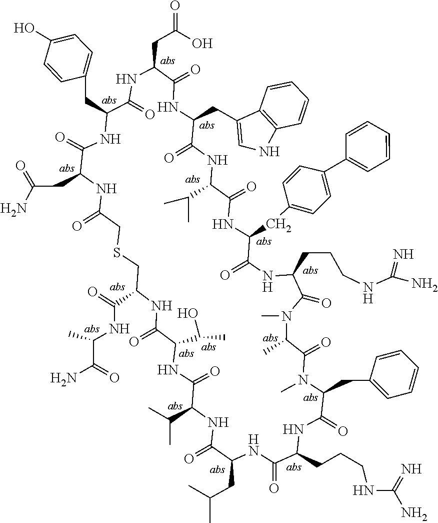

Example 1355 was prepared on a 50 μmol scale. The yield of the product was 2.3 mg, and its estimated purity by LCMS analysis was 100%. Analysis condition A: Retention time=1.84 min: ESI-MS(+) m/z [M+2H]²⁺: 1003.8.

### Preparation of Example 1356

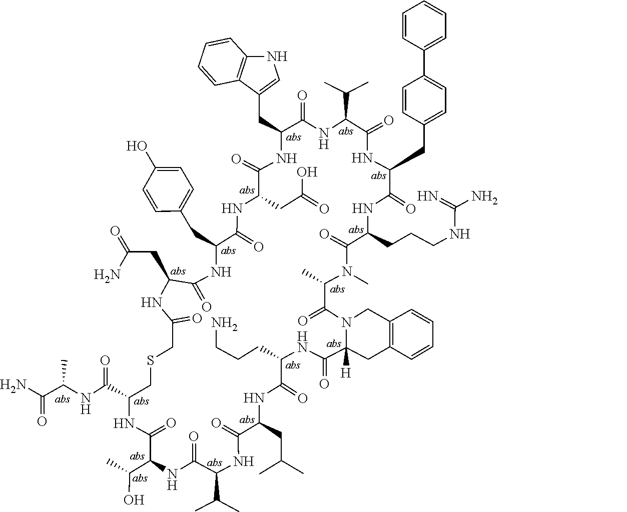

Example 1356 was prepared on a 50 μmol scale. The yield of the product was 9.7 mg, and its estimated purity by LCMS analysis was 88.7%. Analysis condition A: Retention time=1.72 min; ESI-MS(+) m/z [M+H]⁺: 1960.

### Preparation of Example 1357

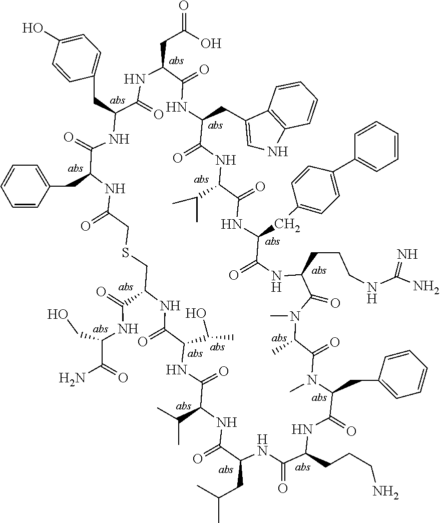

Example 1357 was prepared on a 50 μmol scale. The yield of the product was 15 mg, and its estimated purity by LCMS analysis was 97%. Analysis condition B: Retention time=1.69 min: ESI-MS(+) m/z [M+2H]²⁺: 1006.3.

### Preparation of Example 1358

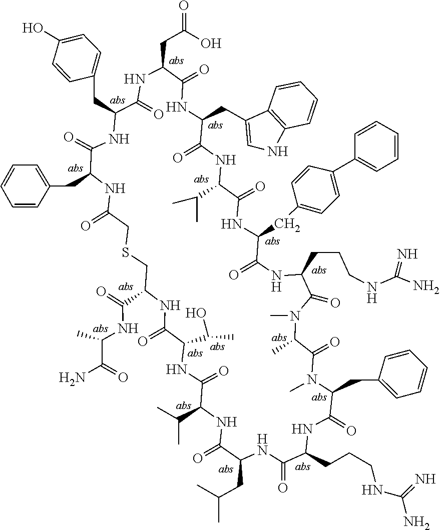

Example 1358 was prepared on a 50 μmol scale. The yield of the product was 26.1 mg, and its estimated purity by LCMS analysis was 86.6%. Analysis condition B: Retention time=1.78 min: ESI-MS(+) m/z [M+2H]²⁺: 1018.9.

### Preparation of Example 1359

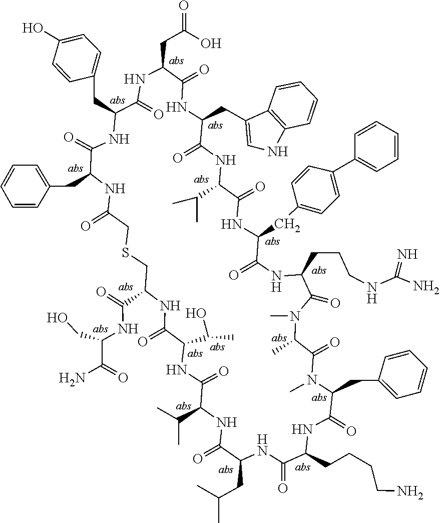

Example 1359 was prepared on a 50 μmol scale. The yield of the product was 1.3 mg, and its estimated purity by LCMS analysis was 98.3%. Analysis condition A: Retention time=1.74 min: ESI-MS(+)m/z [M+2H]²⁺: 1013.1.

### Preparation of Example 1360

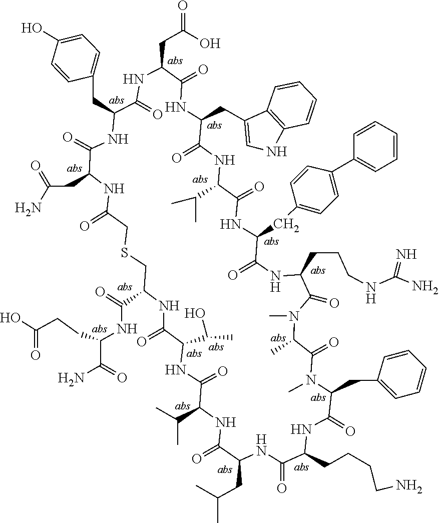

Example 1360 was prepared on a 50 μmol scale. The yield of the product was 2.1 mg, and its estimated purity by LCMS analysis was 97.7%. Analysis condition A: Retention time=1.62 min; ESI-MS(+) m/z [M+2H]²⁺: 1017.8.

### Preparation of Example 1361

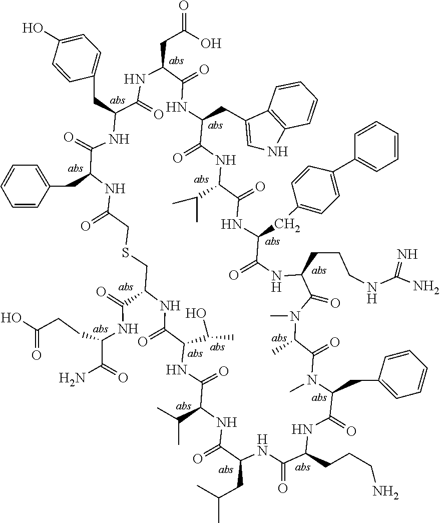

Example 1361 was prepared on a 50 μmol scale. The yield of the product was 3.2 mg, and its estimated purity by LCMS analysis was 100%. Analysis condition A: Retention time=1.72 min: ESI-MS(+) m/z [M+2H]⁺: 1027.1.

### Preparation of Example 1362

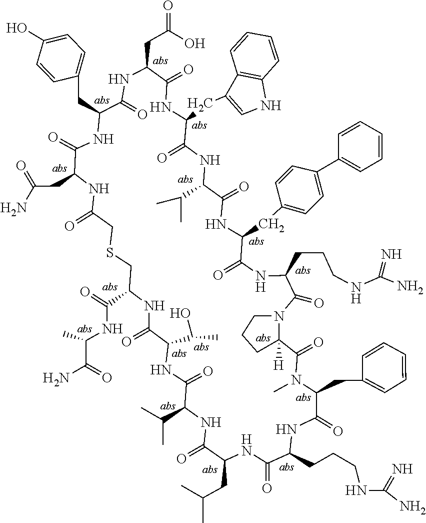

Example 1362 was prepared on a 50 μmol scale. The yield of the product was 11.8 mg, and its estimated purity by LCMS analysis was 93.9%. Analysis condition A: Retention time=1.62, 1.69 min: ESI-MS(+) m/z [M+2H]²⁺: 1009.6.

### Preparation of Example 1363

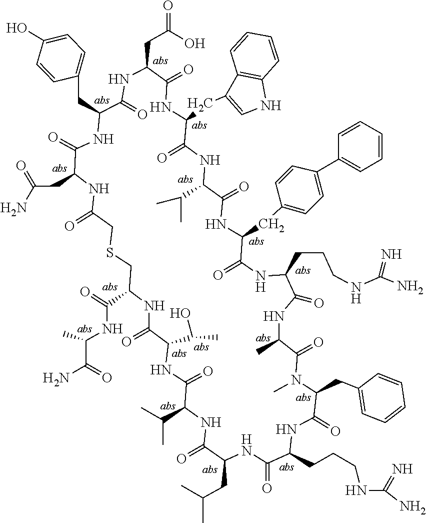

Example 1363 was prepared on a 50 μmol scale. The yield of the product was 29.2 mg, and its estimated purity by LCMS analysis was 97.1%. Analysis condition A: Retention time=1.61 min: ESI-MS(+) m/z [M+2H]²⁺: 996.2.

### Preparation of Example 1364

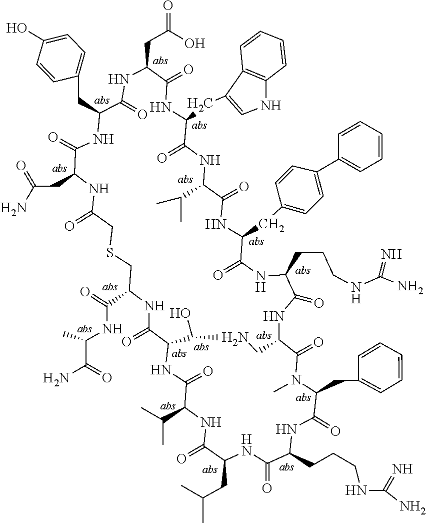

Example 1364 was prepared on a 50 μmol scale. The yield of the product was 37.8 mg, and its estimated purity by LCMS analysis was 100%. Analysis condition A: Retention time=1.46 min: ESI-MS(+) m/z [M+3H]³⁺: 669.6.

### Preparation of Example 1365

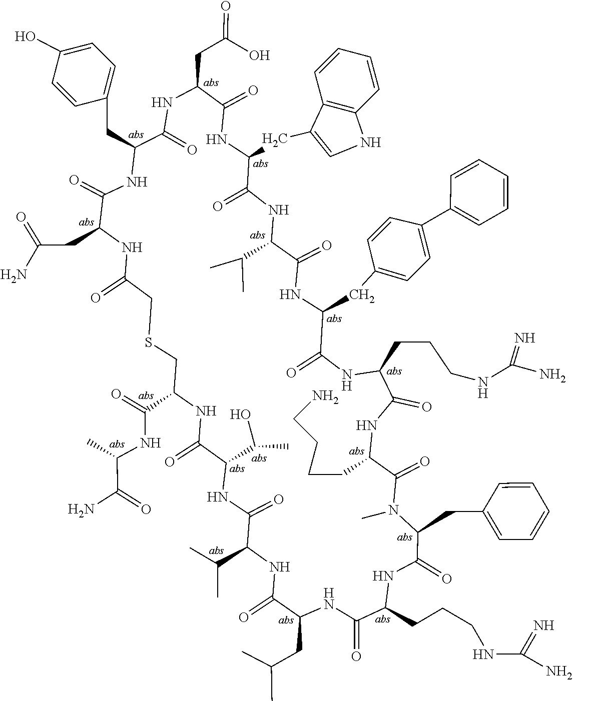

Example 1365 was prepared on a 50 μmol scale. The yield of the product was 5.5 mg, and its estimated purity by LCMS analysis was 94.8%. Analysis condition A: Retention time=2.02 min: ESI-MS(+) m/z [M+3H]³⁺: 682.8.

### Preparation of Example 1366

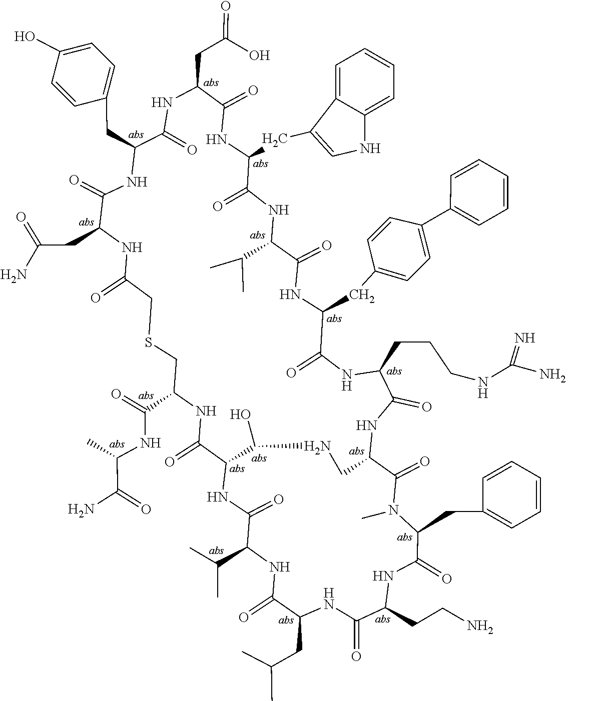

Example 1366 was prepared on a 50 μmol scale. The yield of the product was 30.4 mg, and its estimated purity by LCMS analysis was 98.6%. Analysis condition B: Retention time=1.33 min: ESI-MS(+) m/z [M+3H]³⁺: 650.8.

### Preparation of Example 1367

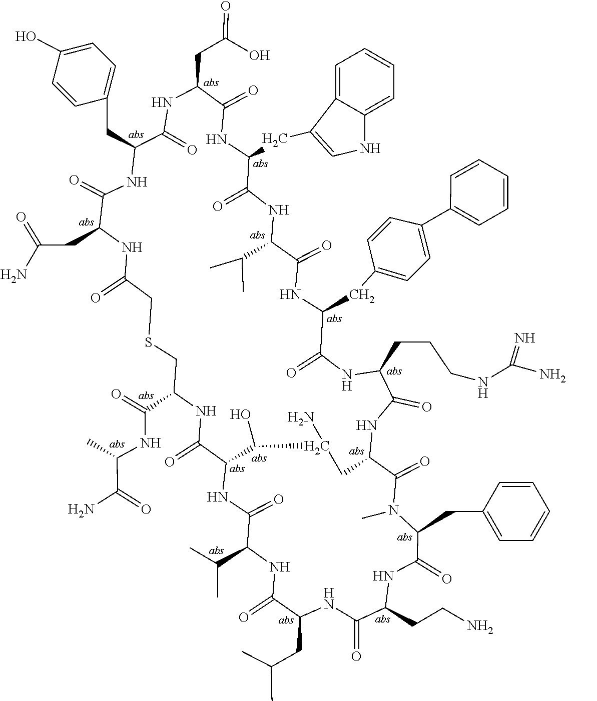

Example 1367 was prepared on a 50 μmol scale. The yield of the product was 12.1 mg, and its estimated purity by LCMS analysis was 100%. Analysis condition A: Retention time=1.36 min: ESI-MS(+) m/z [M+2H]²⁺: 982.1.

### Preparation of Example 1368

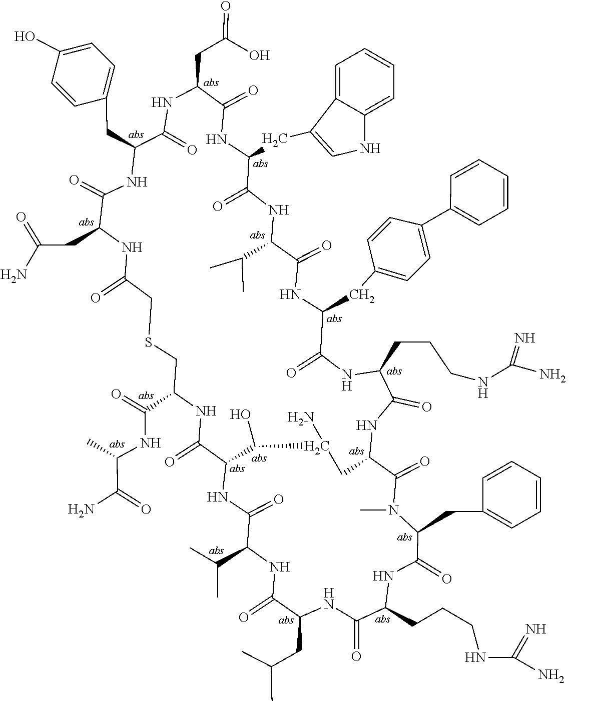

Example 1368 was prepared on a 50 μmol scale. The yield of the product was 18.4 mg, and its estimated purity by LCMS analysis was 87.9%. Analysis condition B: Retention time=1.31 min; ESI-MS(+) m/z [M+3H]³⁺: 674.1.

### Preparation of Example 1369

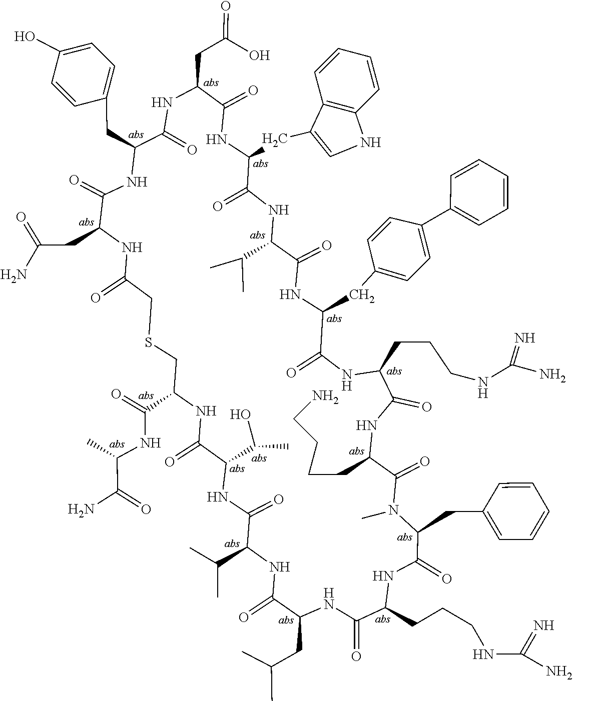

Example 1369 was prepared on a 50 μmol scale. The yield of the product was 14.7 mg, and its estimated purity by LCMS analysis was 95.9%. Analysis condition B: Retention time=1.27 min; ESI-MS(+) m/z [M+3H]³⁺: 683.3.

### Preparation of Example 1370

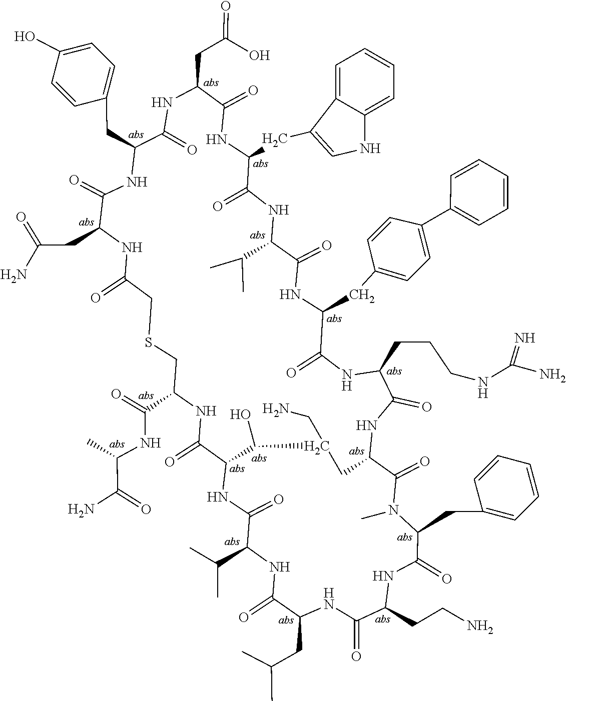

Example 1370 was prepared on a 50 μmol scale. The yield of the product was 9.4 mg, and its estimated purity by LCMS analysis was 97.6%. Analysis condition B: Retention time=1.3 min: ESI-MS(+) m/z [M+3H]³⁺: 660.4.

### Preparation of Example 1371

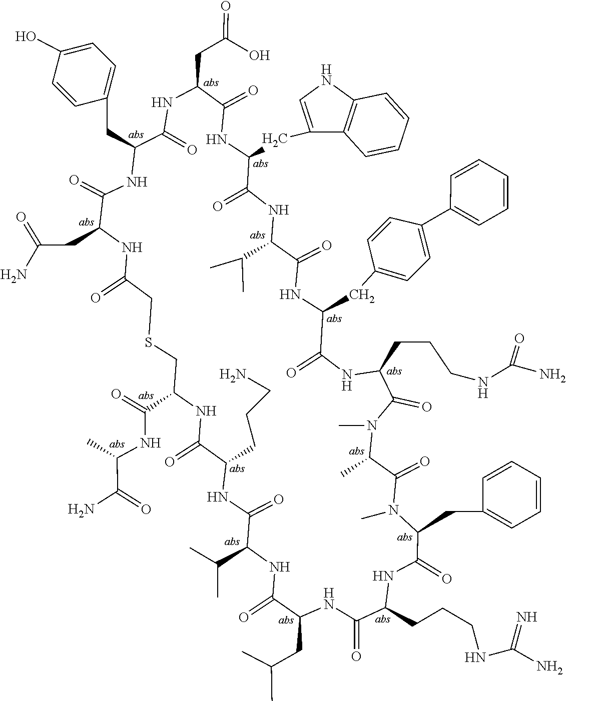

Example 1371 was prepared on a 50 μmol scale. The yield of the product was 8.6 mg, and its estimated purity by LCMS analysis was 91%. Analysis condition B: Retention time=1.65 min; ESI-MS(+) m/z [M+2H]⁺: 1010.1.

### Preparation of Example 1372

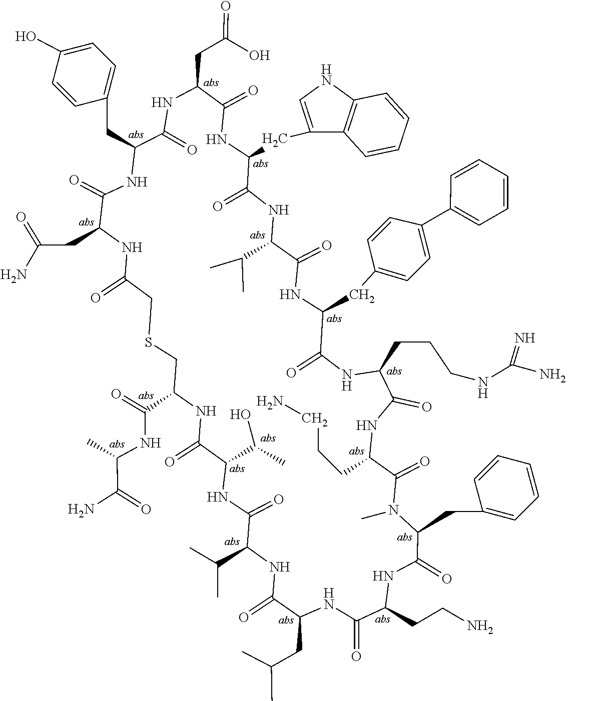

Example 1372 was prepared on a 50 μmol scale. The yield of the product was 11.5 mg, and its estimated purity by LCMS analysis was 100%. Analysis condition A: Retention time=1.44 min: ESI-MS(+) m/z [M+H]⁺: 1992.

### Preparation of Example 1373

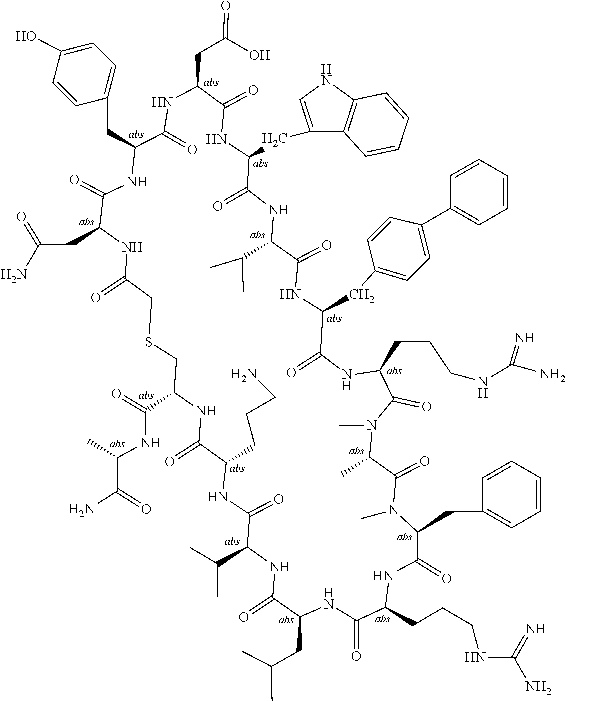

Example 1373 was prepared on a 50 μmol scale. The yield of the product was 15.6 mg, and its estimated purity by LCMS analysis was 97.8%. Analysis condition A: Retention time=1.58 min: ESI-MS(+) m/z [M+2H]²⁺: 1009.2.

### Preparation of Example 1374

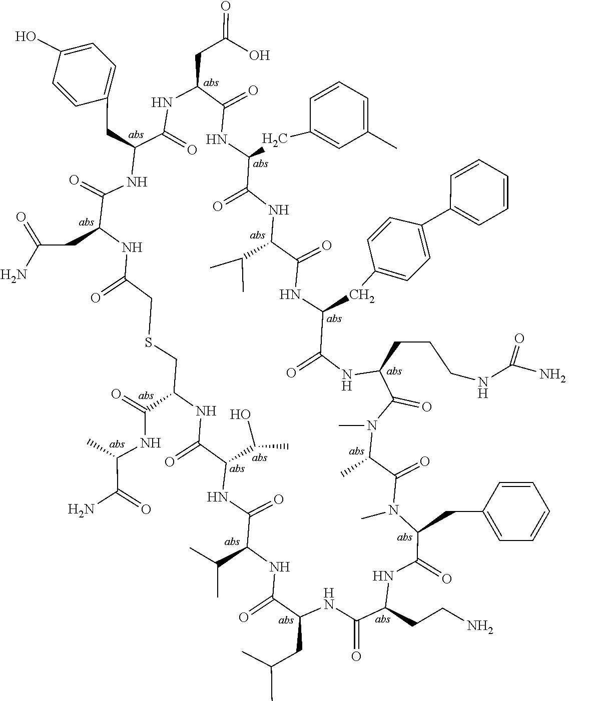

Example 1374 was prepared on a 50 μmol scale. The yield of the product was 4.8 mg, and its estimated purity by LCMS analysis was 99.2%. Analysis condition B: Retention time=1.72, 1.8 min: ESI-MS(+) m/z [M+H]⁺: 1925.

### Preparation of Example 1375

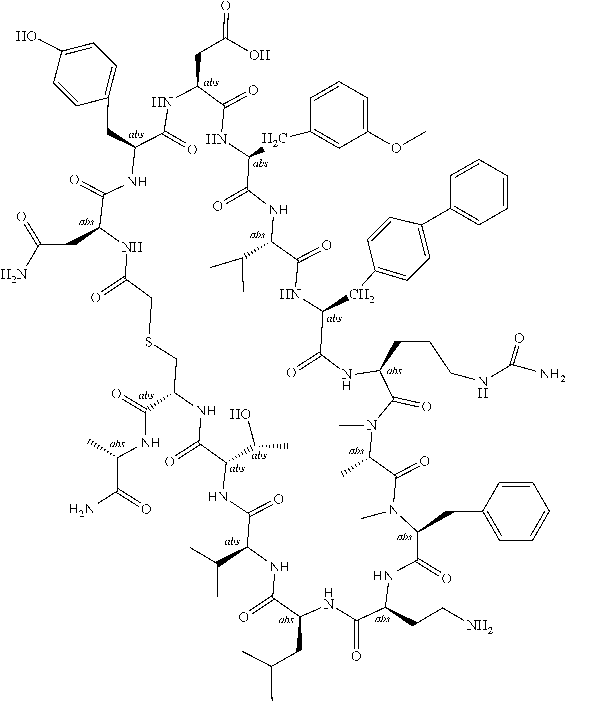

Example 1375 was prepared on a 50 μmol scale. The yield of the product was 4.3 mg, and its estimated purity by LCMS analysis was 93.2%. Analysis condition A: Retention time=1.66 min; ESI-MS(+) m/z [M+H]⁺: 1939.8.

### Preparation of Example 1376

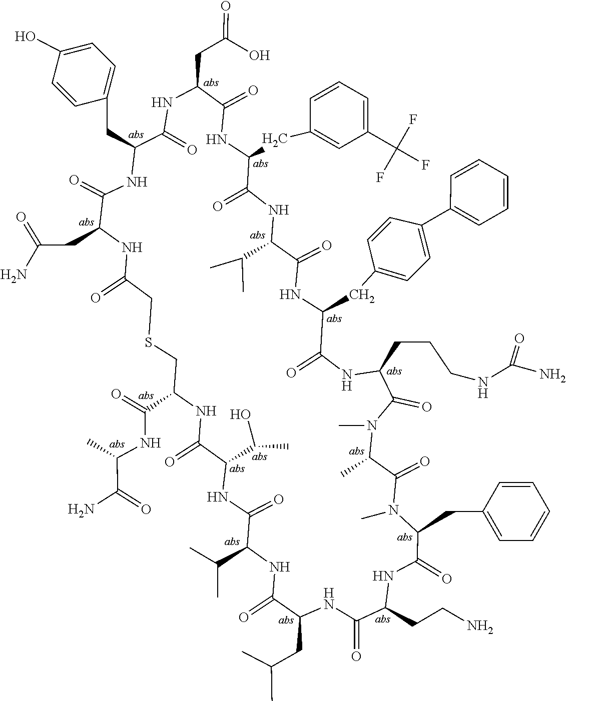

Example 1376 was prepared on a 50 μmol scale. The yield of the product was 3.5 mg, and its estimated purity by LCMS analysis was 99.4%. Analysis condition A: Retention time=1.75 min; ESI-MS(+) m/z [M+H]⁺: 1977.8.

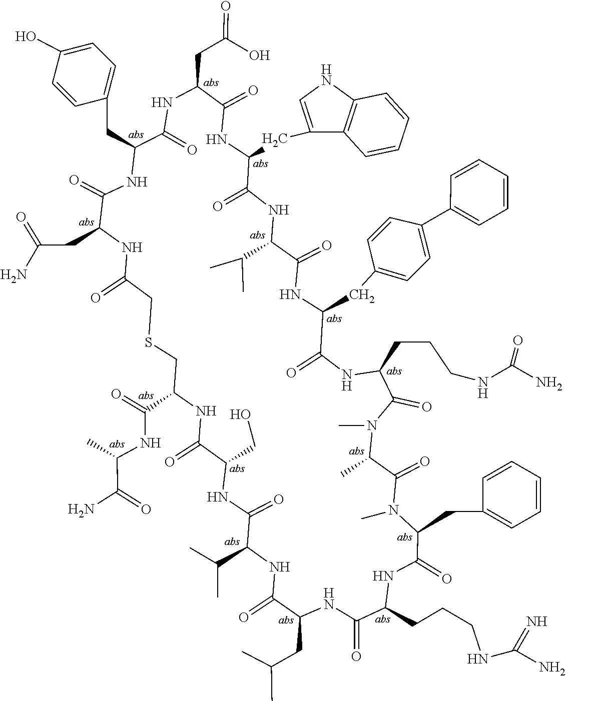

Example 1377 was prepared on a 50 μmol scale. The yield of the product was 3.4 mg, and its estimated purity by LCMS analysis was 86.6%. Analysis condition B: Retention time=1.74 min; ESI-MS(+) m/z [M+H]⁺: 1992.2.

### Preparation of Example 1378

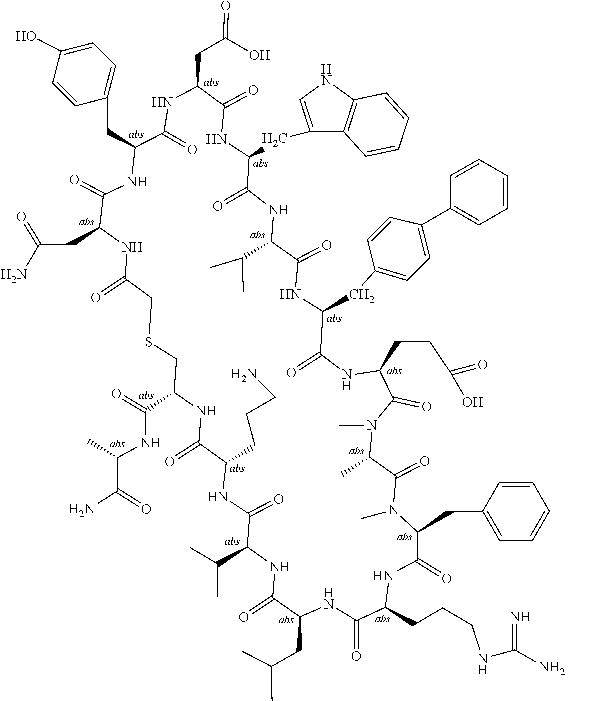

Example 1378 was prepared on a 50 μmol scale. The yield of the product was 5.7 mg, and its estimated purity by LCMS analysis was 93.3%. Analysis condition B: Retention time=1.63 min: ESI-MS(+) m/z [M+2H]⁺: 996.

### Preparation of Example 1379

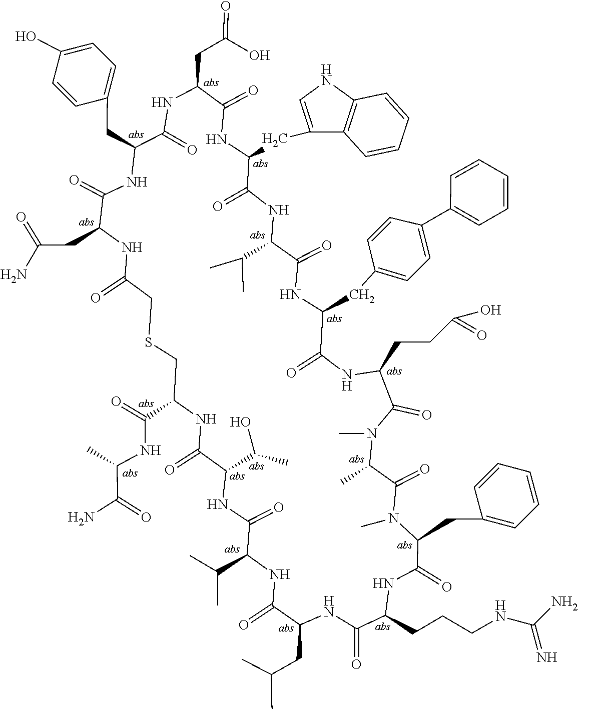

Example 1379 was prepared on a 50 μmol scale. The yield of the product was 14.2 mg, and its estimated purity by LCMS analysis was 86.6%. Analysis condition B: Retention time=1.75 min; ESI-MS(+) m/z [M+2H]²⁺: 989.2.

### Preparation of Example 1380

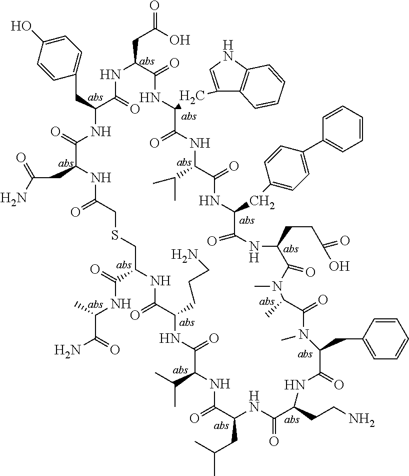 [SMILES: CC(C)C[C@@H]1NC(=O)[C@H](CCN)NC(=O)[C@H](Cc2ccccc2)N(C)C(=O)[C@H](C)N(C)C(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H](CCCN)NC(=O)[C@H](C(C)C)NC1=O]

Example 1380 was prepared on a 50 μmol scale. The yield of the product was 8.5 mg, and its estimated purity by LCMS analysis was 100%. Analysis condition A: Retention time=1.64 min: ESI-MS(+) m/z [M+H]⁺: 1934.

### Preparation of Example 1381

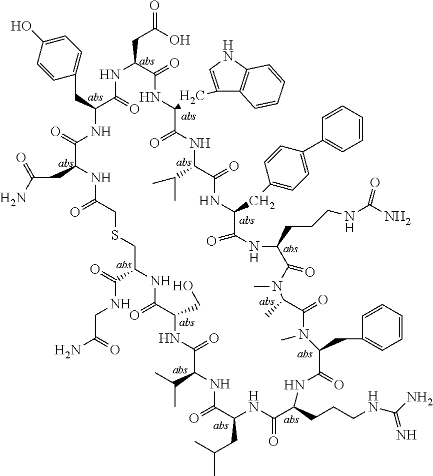 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)NCC(N)=O)NC(=O)[C@H](CO)NC(=O)[C@H](C(C)C)NC1=O]

Example 1381 was prepared on a 50 μmol scale. The yield of the product was 9.2 mg, and its estimated purity by LCMS analysis was 93%. Analysis condition B: Retention time=1.76 min: ESI-MS(+) m/z [M+H]⁺: 1977.

### Preparation of Example 1382

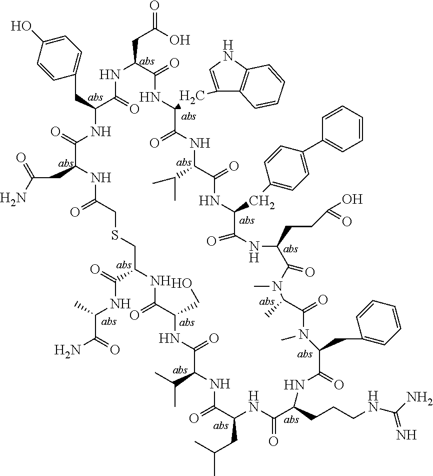 [SMILES: CC(C)C[C@@H]1NC(=O)[C@H](CCCNC(=N)N)NC(=O)[C@H](Cc2ccccc2)N(C)C(=O)[C@H](C)N(C)C(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H](CO)NC(=O)[C@H](C(C)C)NC1=O]

Example 1382 was prepared on a 50 μmol scale. The yield of the product was 1.1 mg, and its estimated purity by LCMS analysis was 100%. Analysis condition A: Retention time=1.65 min; ESI-MS(+) m/z [M+H]⁺: 1963.3.

### Preparation of Example 1383

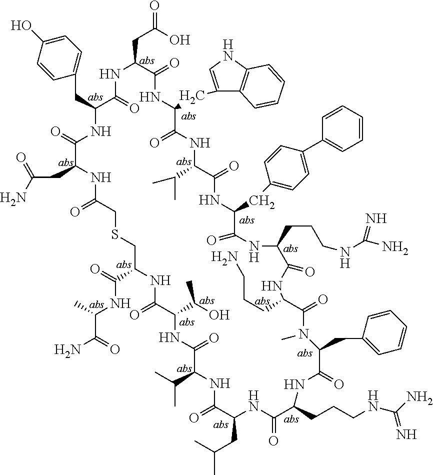 [SMILES: CC(C)C[C@@H]1NC(=O)[C@H](CCCNC(=N)N)NC(=O)[C@H](Cc2ccccc2)N(C)C(=O)[C@H](CCCN)NC(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Example 1383 was prepared on a 50 μmol scale. The yield of the product was 14.1 mg, and its estimated purity by LCMS analysis was 99.2%. Analysis condition A: Retention time=1.55 min; ESI-MS(+) m/z [M+2H]²⁺: 1017.1.

### Preparation of Example 1384

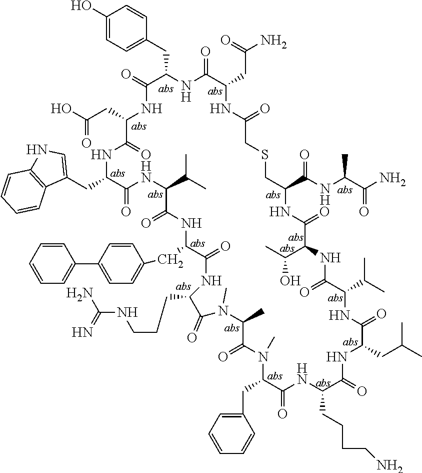 [SMILES: CC(C)C[C@@H]1NC(=O)[C@H](CCCCN)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Example 1384 was prepared on a 50 μmol scale. The yield of the product was 4.4 mg, and its estimated purity by LCMS analysis was 98%. Analysis condition A: Retention time=1.64 min; ESI-MS(+) m/z [M+2H]²⁺: 989.

### Preparation of Example 1385

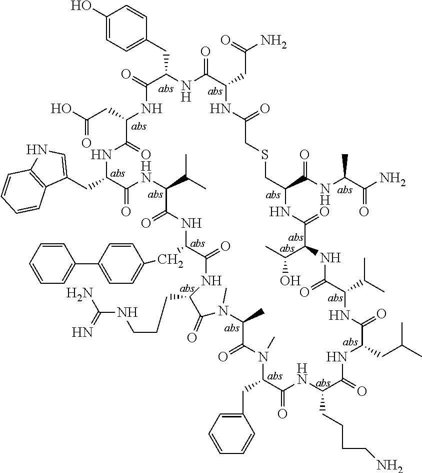 [SMILES: CC(C)C[C@@H]1NC(=O)[C@H](CCCCN)NC(=O)[C@H](Cc2ccccc2)N(C)C(=O)[C@H](C)N(C)C(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Example 1385 was prepared on a 50 μmol scale. The yield of the product was 4.7 mg, and its estimated purity by LCMS analysis was 97.8%. Analysis condition A: Retention time=1.64 min; ESI-MS(+) m/z [M+2H]²⁺: 989.4.

### Preparation of Example 1386

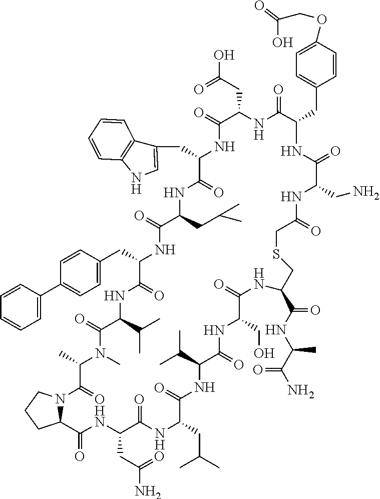 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)[C@H]2CCCN2C(=O)[C@H](C)N(C)C(=O)[C@H](C(C)C)NC(=O)[C@H](Cc2ccc(-c3ccccc3)cc2)NC1=O]

Example 1386 was prepared on a 40 μmol scale. The yield of the product was 8.5 mg, and its estimated purity by LCMS analysis was 92.4%. Analysis condition B: Retention time=1.45 min; ESI-MS(+) m/z [M+2H]²⁺: 936.3.

### Preparation of Example 1387

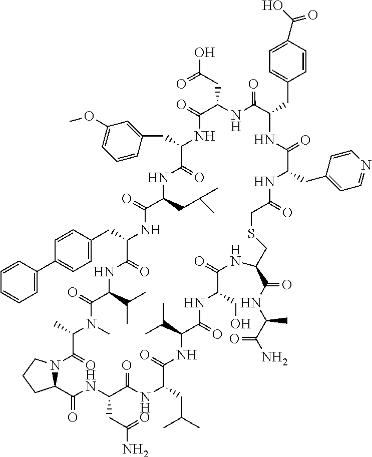 [SMILES: COc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)[C@H]3CCCN3C(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 1387 was prepared on a 40 μmol scale. The yield of the product was 9.2 mg, and its estimated purity by LCMS analysis was 94.2%. Analysis condition B: Retention time=1.45 min; ESI-MS(+)m/z [M+2H]²⁺: 948.

### Preparation of Example 1388

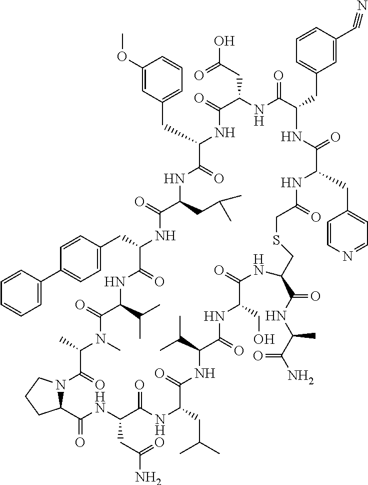 [SMILES: COc1cccc(C[C@@H]2NC(=O)[C@H](CC(=O)O)NC(=O)[C@H](Cc3cccc(C#N)c3)NC(=O)[C@H](Cc3ccncc3)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)[C@H]3CCCN3C(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 1388 was prepared on a 40 μmol scale. The yield of the product was 4.1 mg, and its estimated purity by LCMS analysis was 93.1%. Analysis condition B: Retention time=1.54 min; ESI-MS(+) m/z [M+2H]²: 938.5.

### Preparation of Example 1389

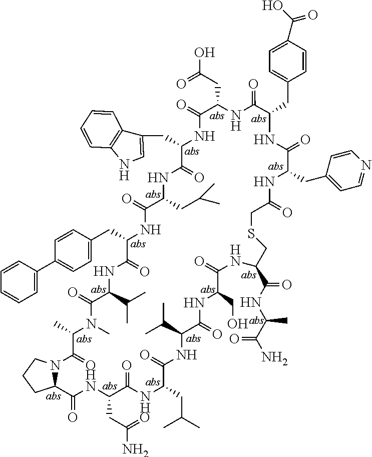 [SMILES: CC(C)C[C@@H]1NC(=O)[C@H](CC(N)=O)NC(=O)[C@H]2CCCN2C(=O)[C@H](C)N(C)C(=O)[C@H](C(C)C)NC(=O)[C@H](Cc2ccc(-c3ccccc3)cc2)NC(=O)[C@@H](CC(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](C)C(N)=O)NC(=O)[C@@H](CO)NC(=O)[C@H](C(C)C)NC1=O]

Example 1389 was prepared on a 40 μmol scale. The yield of the product was 0.6 mg, and its estimated purity by LCMS analysis was 97.7%. Analysis condition B: Retention time=1.46 min; ESI-MS(+) m/z [M+2H]²⁺: 952.

### Preparation of Example 1390

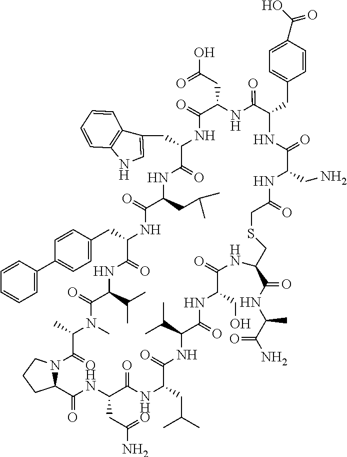 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](CN)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)[C@H]2CCCN2C(=O)[C@H](C)N(C)C(=O)[C@H](C(C)C)NC(=O)[C@H](Cc2ccc(-c3ccccc3)cc2)NC1=O]

Example 1390 was prepared on a 40 μmol scale. The yield of the product was 5.6 mg, and its estimated purity by LCMS analysis was 99%. Analysis condition A: Retention time=1.3 min; ESI-MS(+) m/z [M+2H]²⁺: 921.6.

### Preparation of Example 1391

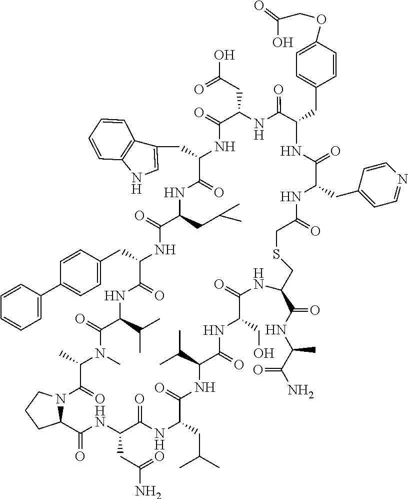 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)[C@H]2CCCN2C(=O)[C@H](C)N(C)C(=O)[C@H](C(C)C)NC(=O)[C@H](Cc2ccc(-c3ccccc3)cc2)NC1=O]

Example 1391 was prepared on a 40 μmol scale. The yield of the product was 4.2 mg, and its estimated purity by LCMS analysis was 97%. Analysis condition A: Retention time=1.26 min; ESI-MS(+) m/z [M+2H]²⁺: 967.3.

### Preparation of Example 1392

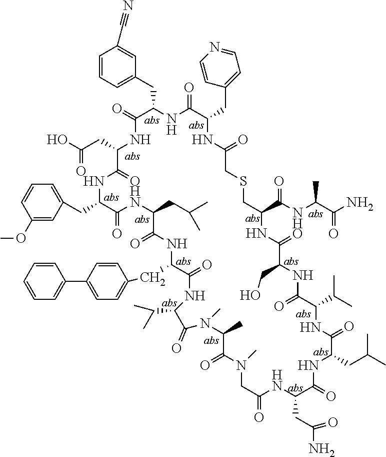 [SMILES: COc1cccc(C[C@@H]2NC(=O)[C@H](CC(=O)O)NC(=O)[C@H](Cc3cccc(C#N)c3)NC(=O)[C@H](Cc3ccncc3)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 1392 was prepared on a 40 μmol scale. The yield of the product was 15.6 mg, and its estimated purity by LCMS analysis was 92%. Analysis condition B: Retention time=1.61 min; ESI-MS(+) m/z [M+2H]²⁺: 925.4.

### Preparation of Example 1393

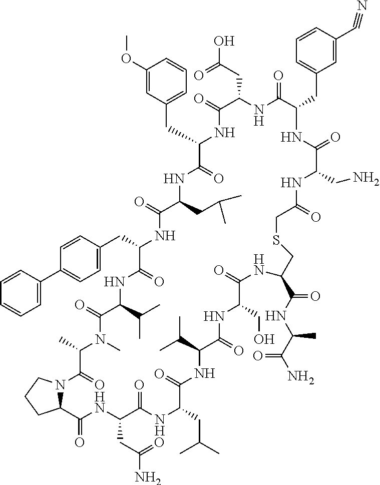 [SMILES: COc1cccc(C[C@@H]2NC(=O)[C@H](CC(=O)O)NC(=O)[C@H](Cc3cccc(C#N)c3)NC(=O)[C@H](CN)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)[C@H]3CCCN3C(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 1393 was prepared on a 40 μmol scale. The yield of the product was 2.9 mg, and its estimated purity by LCMS analysis was 97.5%. Analysis condition A: Retention time=1.5 min: ESI-MS(+) m/z [M+2H]²⁺: 907.3.

### Preparation of Example 1394

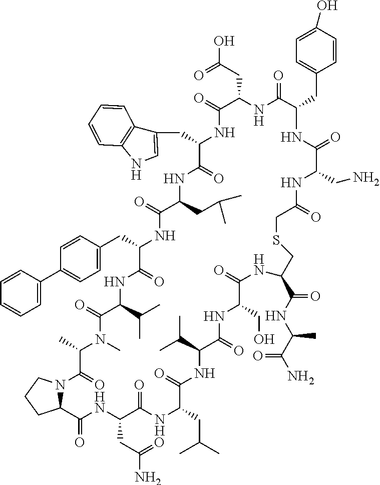 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](CN)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)[C@H]2CCCN2C(=O)[C@H](C)N(C)C(=O)[C@H](C(C)C)NC(=O)[C@H](Cc2ccc(-c3ccccc3)cc2)NC1=O]

Example 1394 was prepared on a 40 μmol scale. The yield of the product was 7.7 mg, and its estimated purity by LCMS analysis was 96.8%. Analysis condition A: Retention time=1.43 min; ESI-MS(+) m/z [M+2H]²⁺: 907.6.

### Preparation of Example 1395

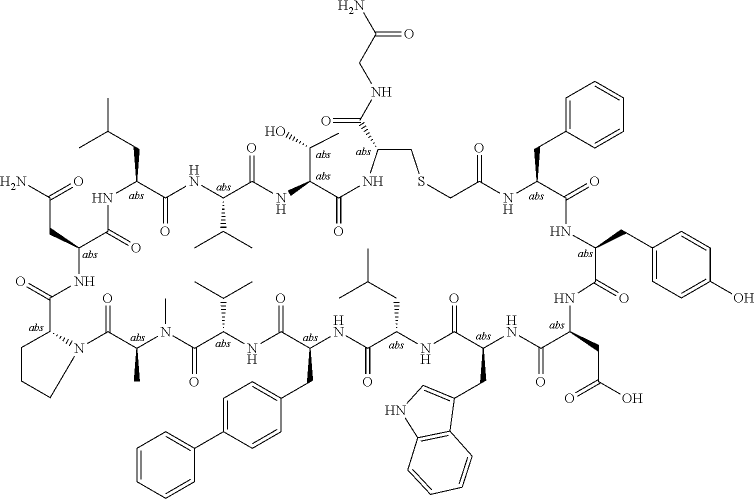

Example 1395 was prepared on a 50 μmol scale. The yield of the product was 16.9 mg, and its estimated purity by LCMS analysis was 98.1%. Analysis condition A: Retention time=1.55 min; ESI-MS(+) m/z [M+2H]²⁺: 938.1.

### Preparation of Example 1396

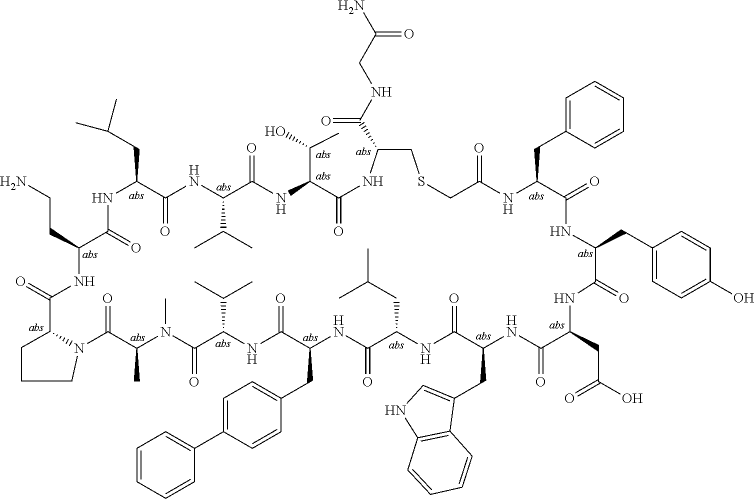

Example 1396 was prepared on a 50 μmol scale. The yield of the product was 13.6 mg, and its estimated purity by LCMS analysis was 100%. Analysis condition A: Retention time=1.52 min; ESI-MS(+) m/z [M+2H]²⁺: 931.1.

### Preparation of Example 1397

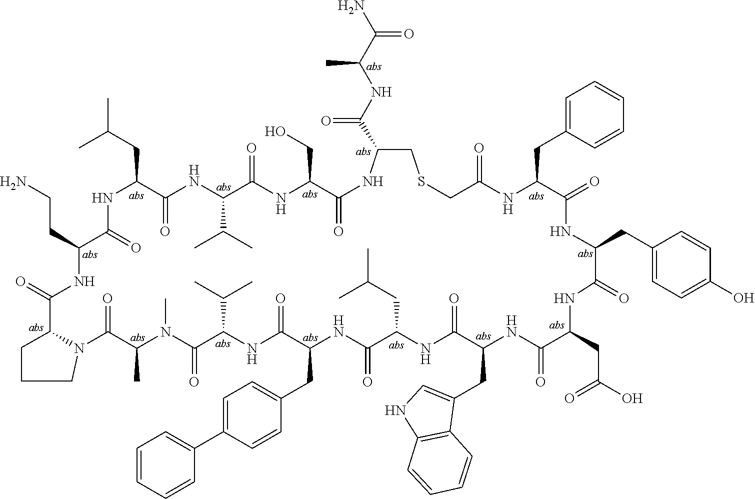

Example 1397 was prepared on a 50 μmol scale. The yield of the product was 6.9 mg, and its estimated purity by LCMS analysis was 98.8%. Analysis condition B: Retention time=1.59 min; ESI-MS(+) m/z [M+2H]²⁺: 930.1.

### Preparation of Example 1398

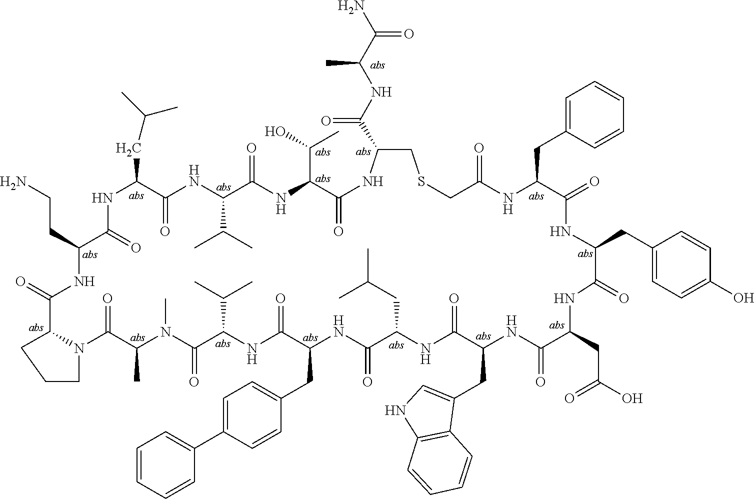

Example 1398 was prepared on a 50 μmol scale. The yield of the product was 8.3 mg, and its estimated purity by LCMS analysis was 93.7%. Analysis condition A: Retention time=1.53 min; ESI-MS(+) m/z [M+2H]²⁻: 938.1.

### Preparation of Example 1399

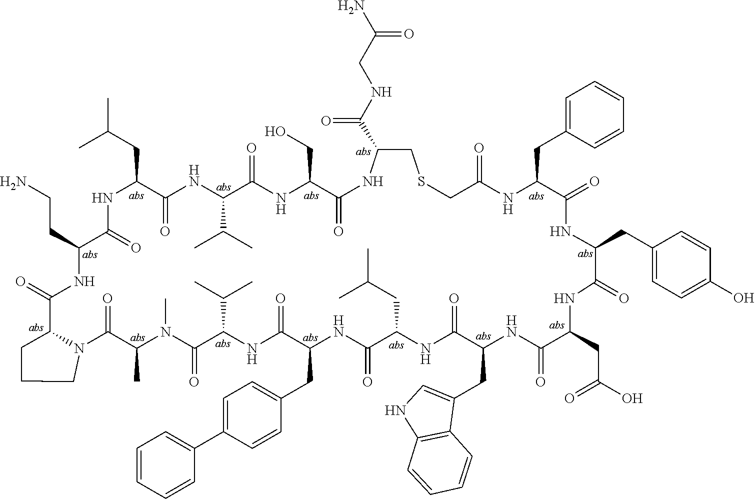

Example 1399 was prepared on a 50 μmol scale. The yield of the product was 7.2 mg, and its estimated purity by LCMS analysis was 98.2%. Analysis condition B: Retention time=1.58 min; ESI-MS(+) m/z [M+2H]²⁺: 924.

### Preparation of Example 1400

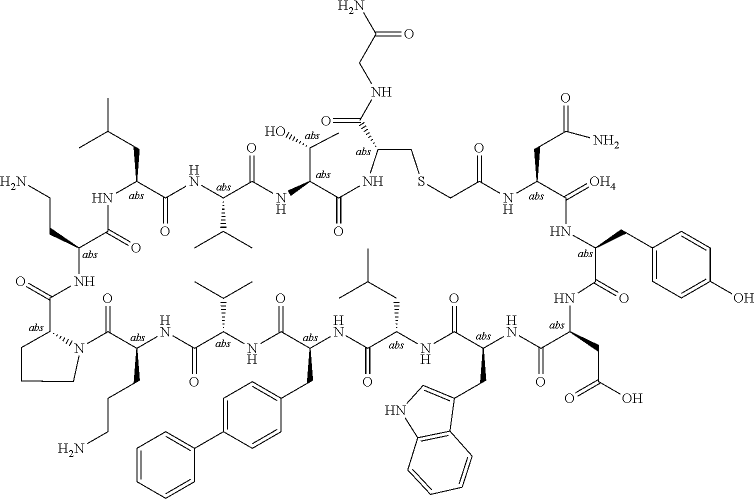 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)NCC(N)=O)NC(=O)[C@H]([C@@H](C)O)NC(=O)[C@H](C(C)C)NC(=O)[C@H](CC(C)C)NC(=O)[C@H](CCN)NC(=O)[C@H]2CCCN2C(=O)[C@H](CCCN)NC(=O)[C@H](C(C)C)NC(=O)[C@H](Cc2ccc(-c3ccccc3)cc2)NC1=O]

Example 1400 was prepared on a 50 μmol scale. The yield of the product was 13.9 mg, and its estimated purity by LCMS analysis was 97.9%. Analysis condition B: Retention time=1.43 min; ESI-MS(+) m/z [M+H]²⁺: 1895.1.

### Preparation of Example 1401

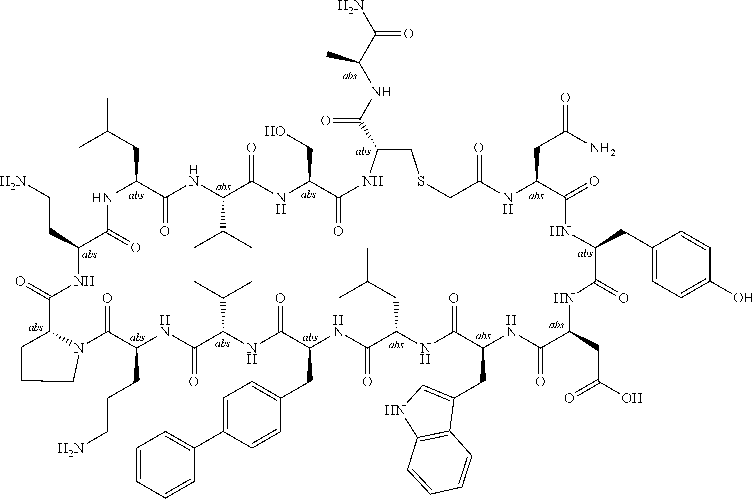 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CCN)NC(=O)[C@H]2CCCN2C(=O)[C@H](CCCN)NC(=O)[C@H](C(C)C)NC(=O)[C@H](Cc2ccc(-c3ccccc3)cc2)NC1=O]

Example 1401 was prepared on a 50 μmol scale. The yield of the product was 1.6 mg, and its estimated purity by LCMS analysis was 94.9%. Analysis condition B: Retention time=1.24 min; ESI-MS(+) m/z [M+2H]²⁺: 928.9.

### Preparation of Example 1402

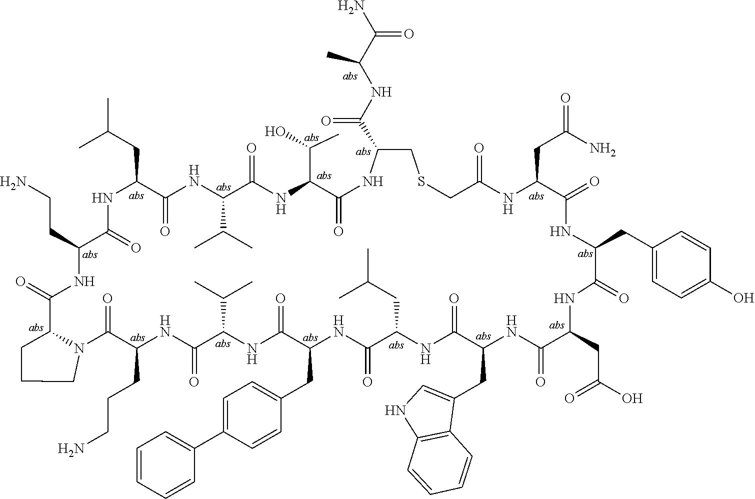 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCN)NC(=O)[C@H]2CCCN2C(=O)[C@H](CCCN)NC(=O)[C@H](C(C)C)NC(=O)[C@H](Cc2ccc(-c3ccccc3)cc2)NC1=O]

Example 1402 was prepared on a 50 μmol scale. The yield of the product was 13.5 mg, and its estimated purity by LCMS analysis was 98.5%. Analysis condition A: Retention time=1.33 min; ESI-MS(+) m/z [M+2H]²⁺: 936.1.

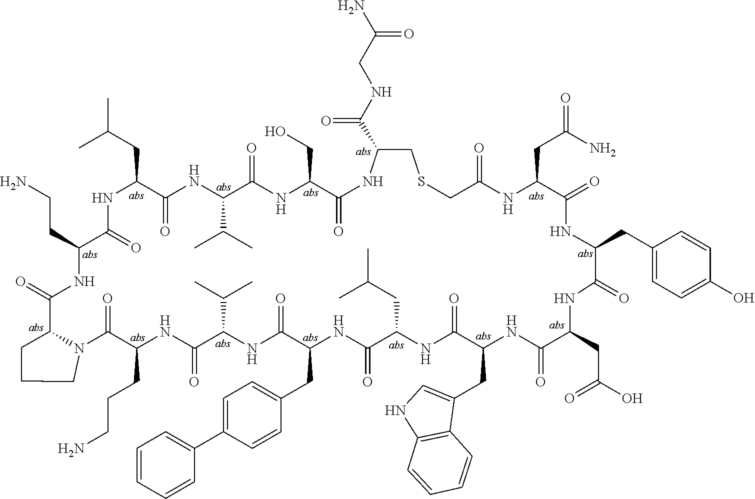 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CCN)NC(=O)[C@H]2CCCN2C(=O)[C@H](CCCN)NC(=O)[C@H](C(C)C)NC(=O)[C@H](Cc2ccc(-c3ccccc3)cc2)NC1=O]

Example 1403 was prepared on a 50 gmol scale. The yield of the product was 14.5 mg, and its estimated purity by LCMS analysis was 93%. Analysis condition B: Retention time=1.28 min; ESI-MS(+) m/z [M+2H]²⁺: 922.2.

### Preparation of Example 1404

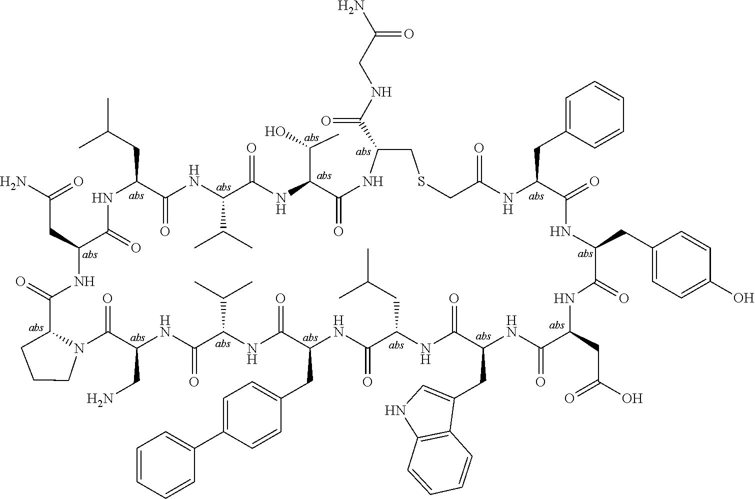 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H]([C@@H](C)O)NC(=O)[C@H](C(C)C)NC(=O)[C@H](CC(C)C)NC(=O)[C@H](CC(N)=O)NC(=O)[C@H]2CCCN2C(=O)[C@H](CN)NC(=O)[C@H](C(C)C)NC(=O)[C@H](Cc2ccc(-c3ccccc3)cc2)NC1=O]

Example 1404 was prepared on a 50 μmol scale. The yield of the product was 35.3 mg, and its estimated purity by LCMS analysis was 95.6%. Analysis condition A: Retention time=1.74 min; ESI-MS(+) m/z [M+2H]²⁺: 938.1.

### Preparation of Example 1405

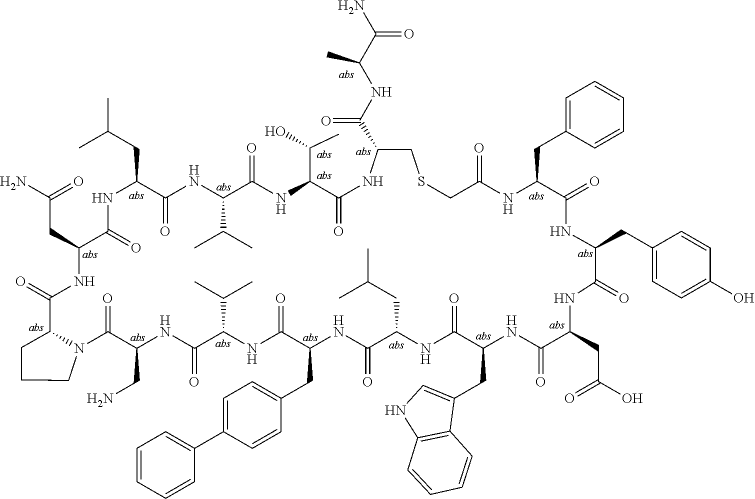 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CC(N)=O)NC(=O)[C@H]2CCCN2C(=O)[C@H](CN)NC(=O)[C@H](C(C)C)NC(=O)[C@H](Cc2ccc(-c3ccccc3)cc2)NC1=O]

Example 1405 was prepared on a 50 μmol scale. The yield of the product was 10.9 mg, and its estimated purity by LCMS analysis was 93.7%. Analysis condition A: Retention time=1.76 min; ESI-MS(+) m/z [M+2H]²⁺: 945.1.

### Preparation of Example 1406

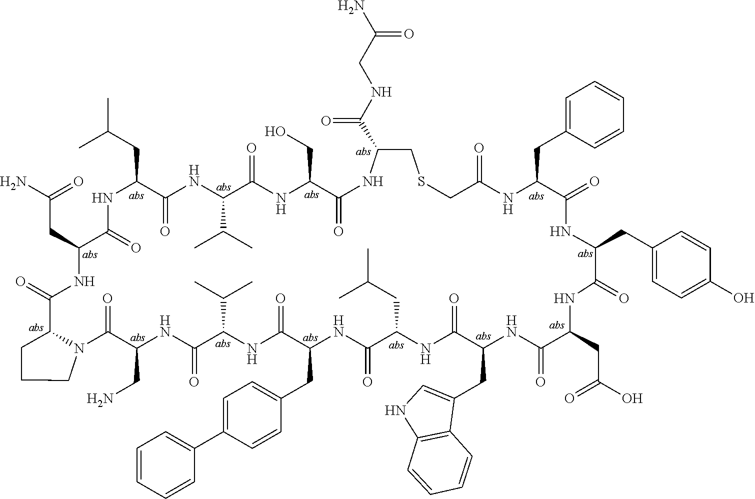 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CC(N)=O)NC(=O)[C@H]2CCCN2C(=O)[C@H](CN)NC(=O)[C@H](C(C)C)NC(=O)[C@H](Cc2ccc(-c3ccccc3)cc2)NC1=O]

Example 1406 was prepared on a 50 μmol scale. The yield of the product was 10.5 mg, and its estimated purity by LCMS analysis was 93.5%. Analysis condition A: Retention time=1.71 min; ESI-MS(+) m/z [M+2H]²⁺.

### Preparation of Example 1407

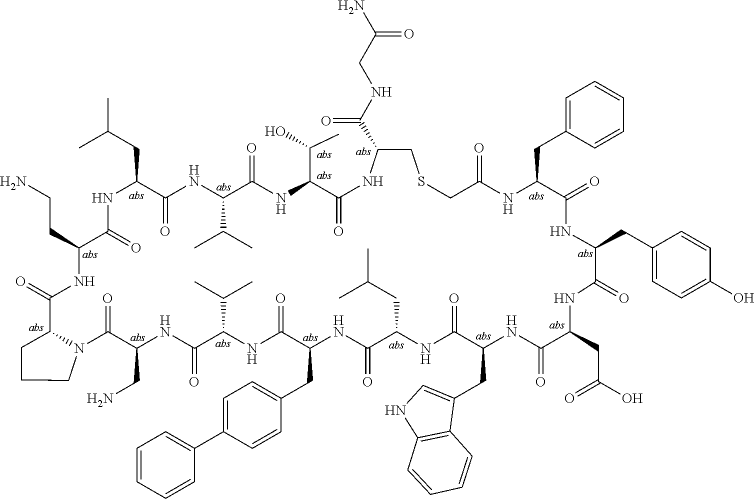 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H]([C@@H](C)O)NC(=O)[C@H](C(C)C)NC(=O)[C@H](CC(C)C)NC(=O)[C@H](CCN)NC(=O)[C@H]2CCCN2C(=O)[C@H](CN)NC(=O)[C@H](C(C)C)NC(=O)[C@H](Cc2ccc(-c3ccccc3)cc2)NC1=O]

Example 1407 was prepared on a 50 μmol scale. The yield of the product was 14.4 mg, and its estimated purity by LCMS analysis was 92.4%. Analysis condition B: Retention time=1.45 min; ESI-MS(+) m/z [M+2H]²⁺: 930.9.

### Preparation of Example 1408

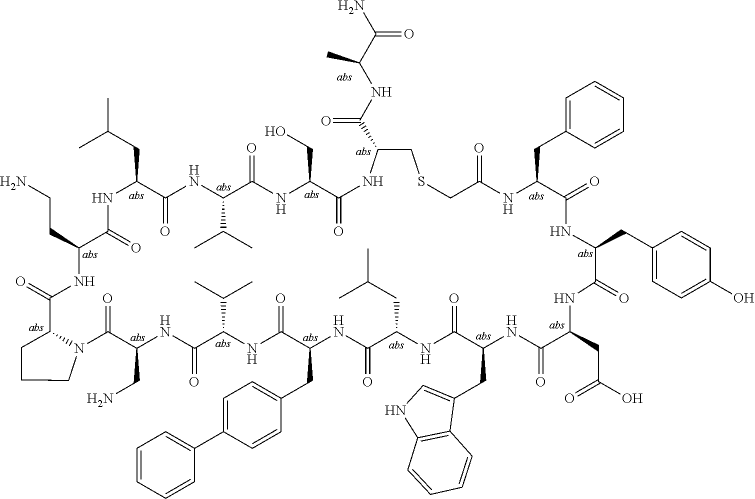 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CCN)NC(=O)[C@H]2CCCN2C(=O)[C@H](CN)NC(=O)[C@H](C(C)C)NC(=O)[C@H](Cc2ccc(-c3ccccc3)cc2)NC1=O]

Example 1408 was prepared on a 50 μmol scale. The yield of the product was 5.3 mg, and its estimated purity by LCMS analysis was 97.9%. Analysis condition A: Retention time=1.55 min; ESI-MS(+) m/z [M+H]⁺: 1861.5.

### Preparation of Example 1409

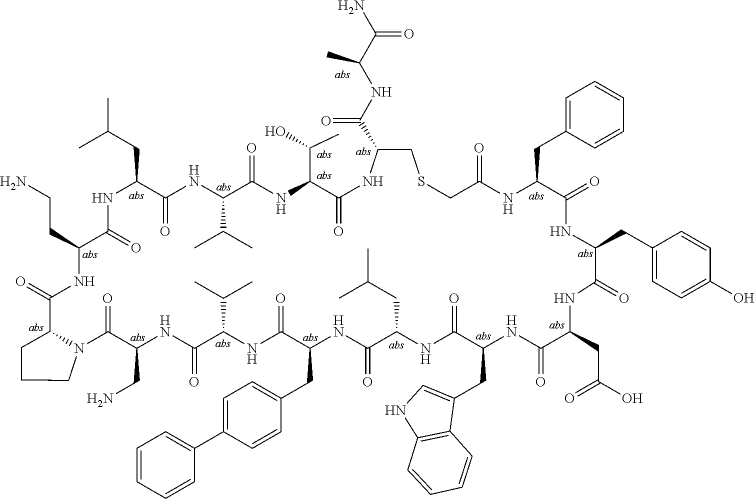 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCN)NC(=O)[C@H]2CCCN2C(=O)[C@H](CN)NC(=O)[C@H](C(C)C)NC(=O)[C@H](Cc2ccc(-c3ccccc3)cc2)NC1=O]

Example 1409 was prepared on a 50 μmol scale. The yield of the product was 2.6 mg, and its estimated purity by LCMS analysis was 95.9%. Analysis condition A: Retention time=1.59 min; ESI-MS(+) m/z [M+2H]²⁺: 938.

### Preparation of Example 1410

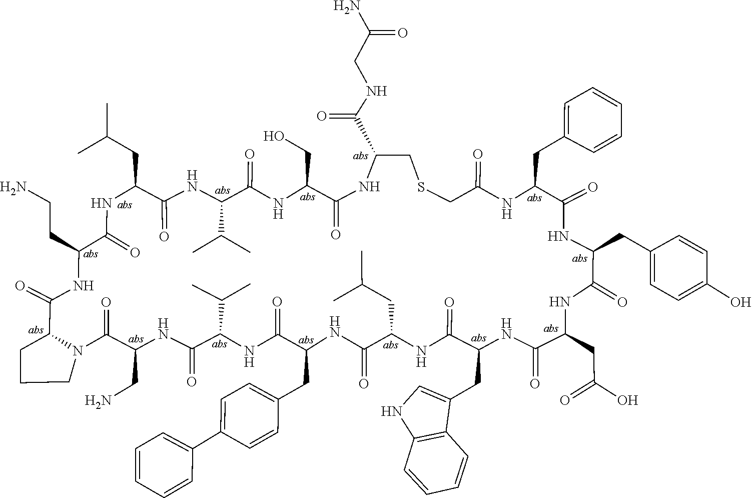 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CCN)NC(=O)[C@H]2CCCN2C(=O)[C@H](CN)NC(=O)[C@H](C(C)C)NC(=O)[C@H](Cc2ccc(-c3ccccc3)cc2)NC1=O]

Example 1410 was prepared on a 50 μmol scale. The yield of the product was 21.2 mg, and its estimated purity by LCMS analysis was 93.1%. Analysis condition B: Retention time=1.43 min; ESI-MS(+) m/z [M+2H]²⁺: 924.1.

### Preparation of Example 1411

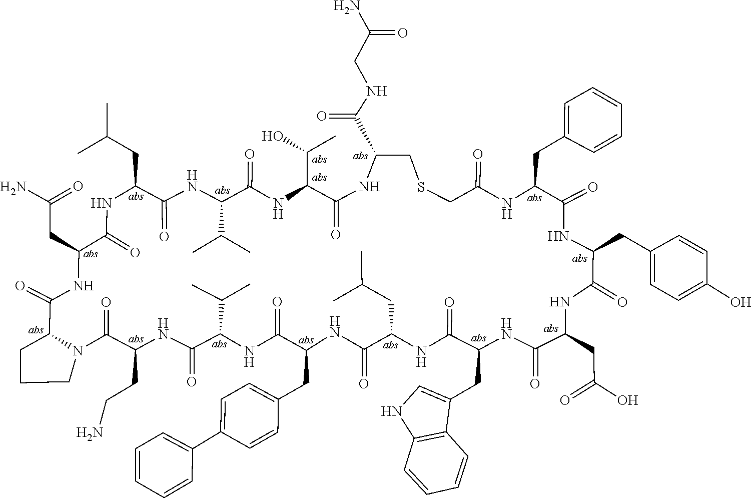 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H]([C@@H](C)O)NC(=O)[C@H](C(C)C)NC(=O)[C@H](CC(C)C)NC(=O)[C@H](CC(N)=O)NC(=O)[C@H]2CCCN2C(=O)[C@H](CCN)NC(=O)[C@H](C(C)C)NC(=O)[C@H](Cc2ccc(-c3ccccc3)cc2)NC1=O]

Example 1411 was prepared on a 50 μmol scale. The yield of the product was 13.4 mg, and its estimated purity by LCMS analysis was 97.8%. Analysis condition B: Retention time=1.57 min; ESI-MS(+) m/z [M+2H]²⁺: 945.5.

### Preparation of Example 1412

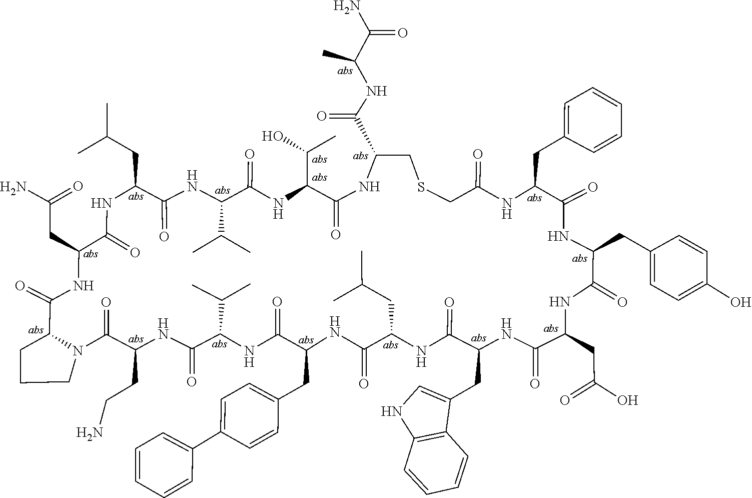 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CC(N)=O)NC(=O)[C@H]2CCCN2C(=O)[C@H](CCN)NC(=O)[C@H](C(C)C)NC(=O)[C@H](Cc2ccc(-c3ccccc3)cc2)NC1=O]

Example 1412 was prepared on a 50 μmol scale. The yield of the product was 12.7 mg, and its estimated purity by LCMS analysis was 94.9%. Analysis condition A: Retention time=1.66 min; ESI-MS(+) m/z [M+2H]²⁺: 952.1.

### Preparation of Example 1413

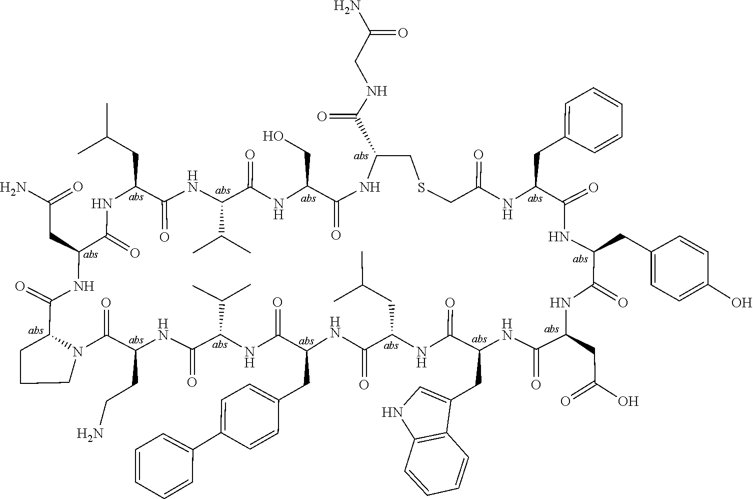 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CC(N)=O)NC(=O)[C@H]2CCCN2C(=O)[C@H](CCN)NC(=O)[C@H](C(C)C)NC(=O)[C@H](Cc2ccc(-c3ccccc3)cc2)NC1=O]

Example 1413 was prepared on a 50 μmol scale. The yield of the product was 16.6 mg, and its estimated purity by LCMS analysis was 98.7%. Analysis condition A: Retention time=1.62 min; ESI-MS(+) m/z [M+2H]²⁺: 938.1.

### Preparation of Example 1414

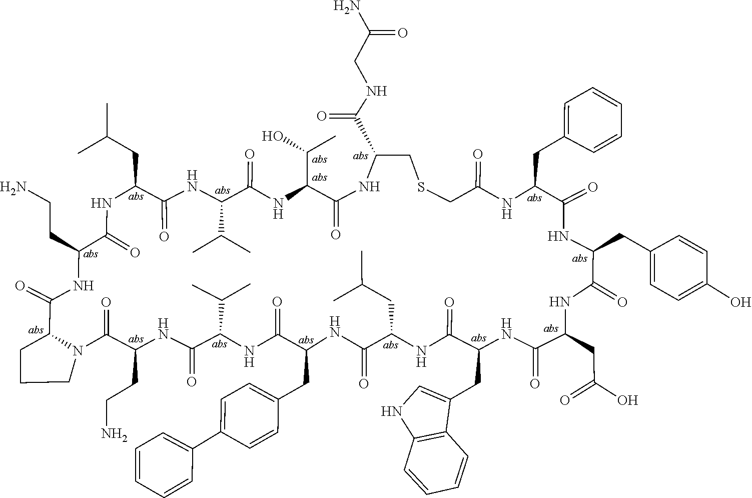 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H]([C@@H](C)O)NC(=O)[C@H](C(C)C)NC(=O)[C@H](CC(C)C)NC(=O)[C@H](CCN)NC(=O)[C@H]2CCCN2C(=O)[C@H](CCN)NC(=O)[C@H](C(C)C)NC(=O)[C@H](Cc2ccc(-c3ccccc3)cc2)NC1=O]

Example 1414 was prepared on a 50 μmol scale. The yield of the product was 6.9 mg, and its estimated purity by LCMS analysis was 97.3%. Analysis condition B: Retention time=1.45 min; ESI-MS(+)m/z [M+2H]²⁺: 938.2.

### Preparation of Example 141S

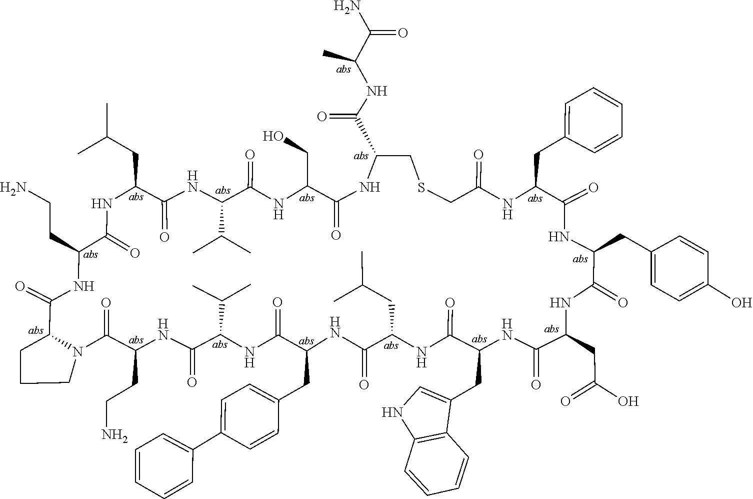

Example 1415 was prepared on a 50 μmol scale. The yield of the product was 6.1 mg, and its estimated purity by LCMS analysis was 94.4%. Analysis condition B: Retention time=1.46 min; ESI-MS(+) m/z [M+2H]²⁺: 938.

### Preparation of Example 1416

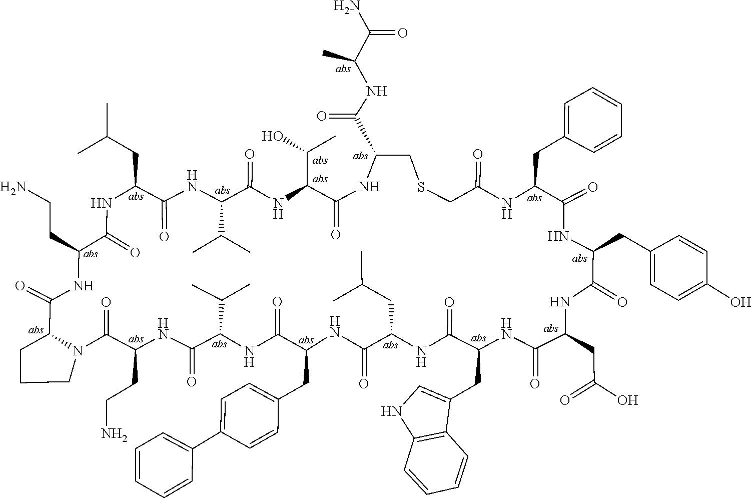 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCN)NC(=O)[C@H]2CCCN2C(=O)[C@H](CCN)NC(=O)[C@H](C(C)C)NC(=O)[C@H](Cc2ccc(-c3ccccc3)cc2)NC1=O]

Example 1416 was prepared on a 50 μmol scale. The yield of the product was 9.3 mg, and its estimated purity by LCMS analysis was 95.9%. Analysis condition B: Retention time=1.45 min; ESI-MS(+) m/z [M+2H]²⁺: 945.4.

### Preparation of Example 1417

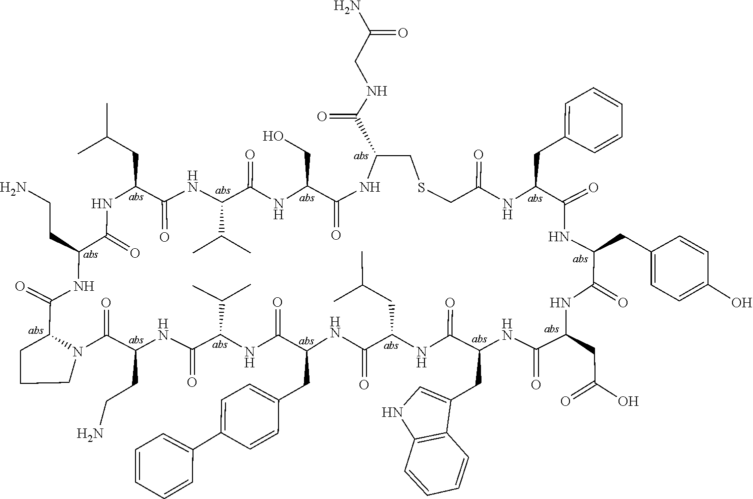 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CCN)NC(=O)[C@H]2CCCN2C(=O)[C@H](CCN)NC(=O)[C@H](C(C)C)NC(=O)[C@H](Cc2ccc(-c3ccccc3)cc2)NC1=O]

Example 1417 was prepared on a 50 μmol scale. The yield of the product was 17.4 mg, and its estimated purity by LCMS analysis was 93.9%. Analysis condition B: Retention time=1.41, 1.46 min; ESI-MS(+) m/z [M+H]⁺: 1862.

### Preparation of Example 1418

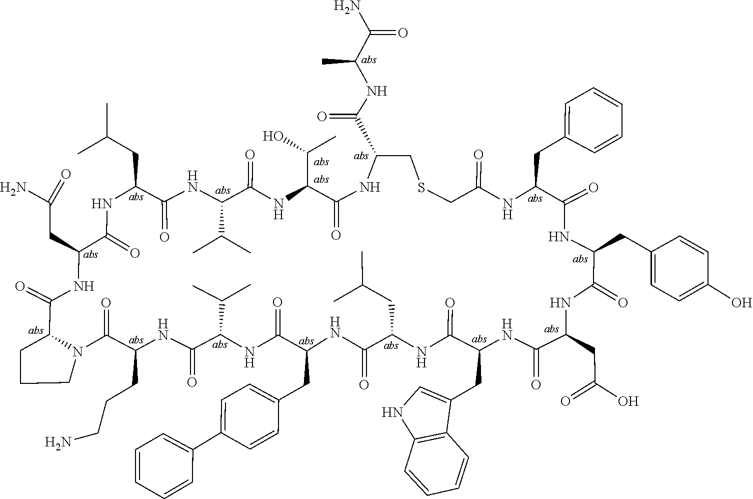 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CC(N)=O)NC(=O)[C@H]2CCCN2C(=O)[C@H](CCCN)NC(=O)[C@H](C(C)C)NC(=O)[C@H](Cc2ccc(-c3ccccc3)cc2)NC1=O]

Example 1418 was prepared on a 50 μmol scale. The yield of the product was 24.5 mg, and its estimated purity by LCMS analysis was 93.2%. Analysis condition B: Retention time=1.57 min; ESI-MS(+) m/z [M+2H]²⁺: 959.2.

### Preparation of Example 1419

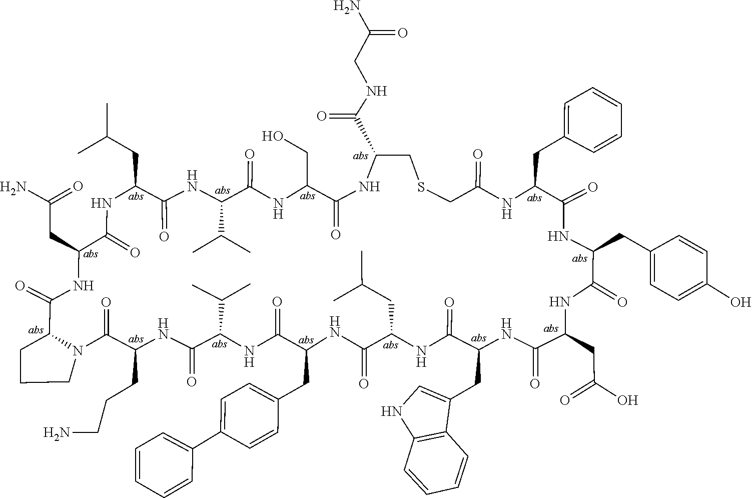 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)C(CO)NC(=O)[C@H](C(C)C)NC(=O)[C@H](CC(C)C)NC(=O)[C@H](CC(N)=O)NC(=O)[C@H]2CCCN2C(=O)[C@H](CCCN)NC(=O)[C@H](C(C)C)NC(=O)[C@H](Cc2ccc(-c3ccccc3)cc2)NC1=O]

Example 1419 was prepared on a 50 μmol scale. The yield of the product was 25.4 mg, and its estimated purity by LCMS analysis was 98.7%. Analysis condition B: Retention time=1.59 min; ESI-MS(+) m/z [M+2H]²⁺: 945.1.

### Preparation of Example 1420

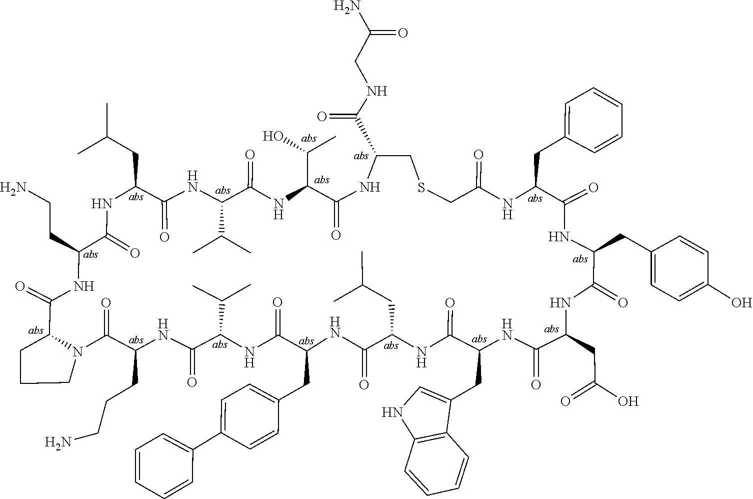 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H]([C@@H](C)O)NC(=O)[C@H](C(C)C)NC(=O)[C@H](CC(C)C)NC(=O)[C@H](CCN)NC(=O)[C@H]2CCCN2C(=O)[C@H](CCCN)NC(=O)[C@H](C(C)C)NC(=O)[C@H](Cc2ccc(-c3ccccc3)cc2)NC1=O]

Example 1420 was prepared on a 50 μmol scale. The yield of the product was 25.9 mg, and its estimated purity by LCMS analysis was 97.5%. Analysis condition A: Retention time=1.62 min; ESI-MS(+) m/z [M+2H]²⁺: 945.1.

### Preparation of Example 1421

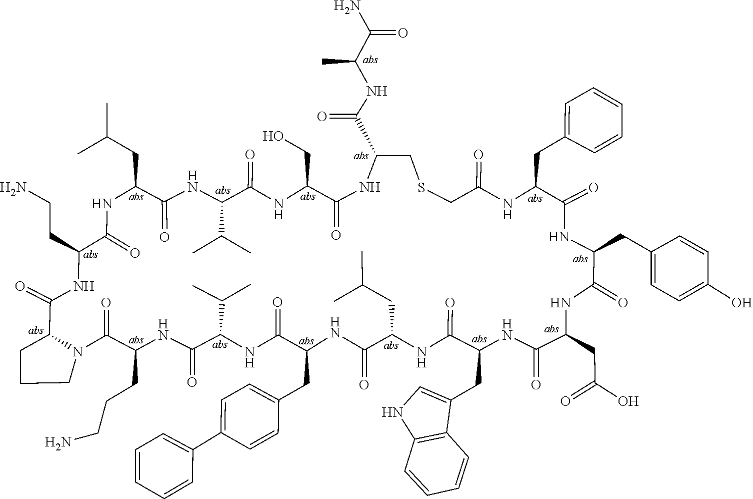 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CCN)NC(=O)[C@H]2CCCN2C(=O)[C@H](CCCN)NC(=O)[C@H](C(C)C)NC(=O)[C@H](Cc2ccc(-c3ccccc3)cc2)NC1=O]

Example 1421 was prepared on a 50 μmol scale. The yield of the product was 13.3 mg, and its estimated purity by LCMS analysis was 97%. Analysis condition A: Retention time=1.5 min; ESI-MS(+) m/z [M+H]⁺: 1889.8.

### Preparation of Example 1422

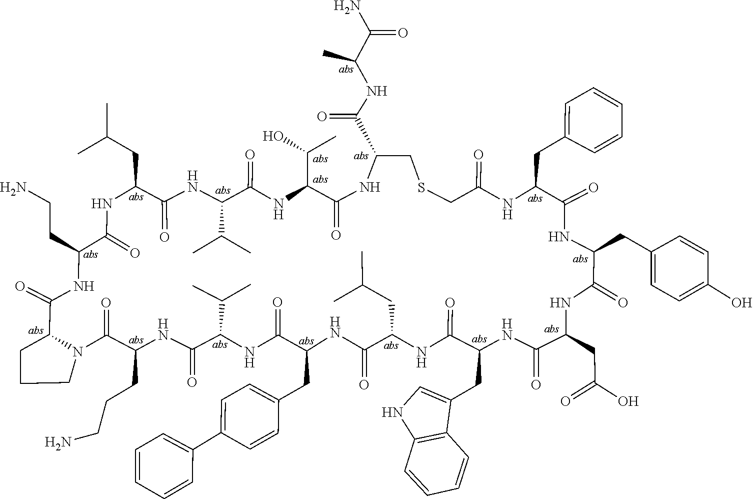 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCN)NC(=O)[C@H]2CCCN2C(=O)[C@H](CCCN)NC(=O)[C@H](C(C)C)NC(=O)[C@H](Cc2ccc(-c3ccccc3)cc2)NC1=O]

Example 1422 was prepared on a 50 μmol scale. The yield of the product was 30.1 mg, and its estimated purity by LCMS analysis was 98.3%. Analysis condition A: Retention time=1.62 min; ESI-MS(+) m/z [M+H]⁺: 1904.1.

### Preparation of Example 1423

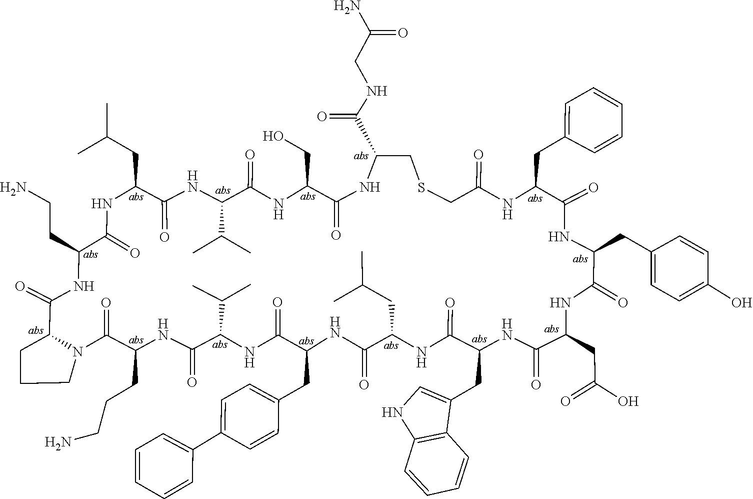 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CCN)NC(=O)[C@H]2CCCN2C(=O)[C@H](CCCN)NC(=O)[C@H](C(C)C)NC(=O)[C@H](Cc2ccc(-c3ccccc3)cc2)NC1=O]

Example 1423 was prepared on a 50 μmol scale. The yield of the product was 23.5 mg, and its estimated purity by LCMS analysis was 99.1%. Analysis condition A: Retention time=1.49 min; ESI-MS(+) m/z [M+H]⁺: 1875.9.

### Preparation of Example 1424

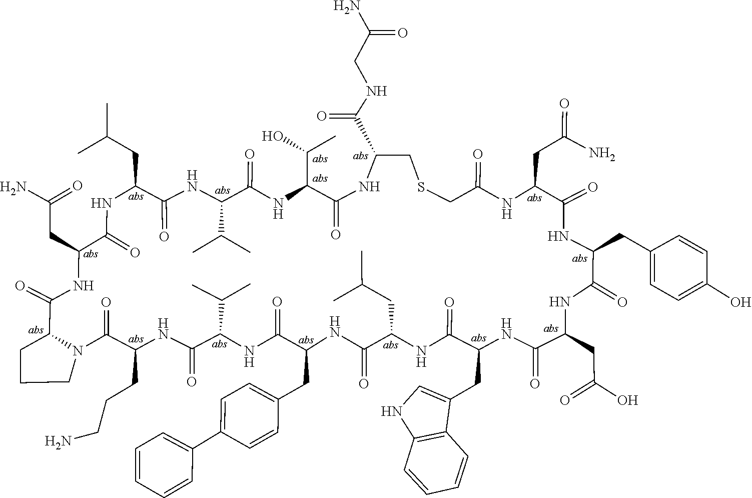 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)NCC(N)=O)NC(=O)[C@H]([C@@H](C)O)NC(=O)[C@H](C(C)C)NC(=O)[C@H](CC(C)C)NC(=O)[C@H](CC(N)=O)NC(=O)[C@H]2CCCN2C(=O)[C@H](CCCN)NC(=O)[C@H](C(C)C)NC(=O)[C@H](Cc2ccc(-c3ccccc3)cc2)NC1=O]

Example 1424 was prepared on a 50 μmol scale. The yield of the product was 10.6 mg, and its estimated purity by LCMS analysis was 94.4%. Analysis condition B: Retention time=1.44 min; ESI-MS(+) m/z [M+2H]²⁺: 936.1.

### Preparation of Example 1425

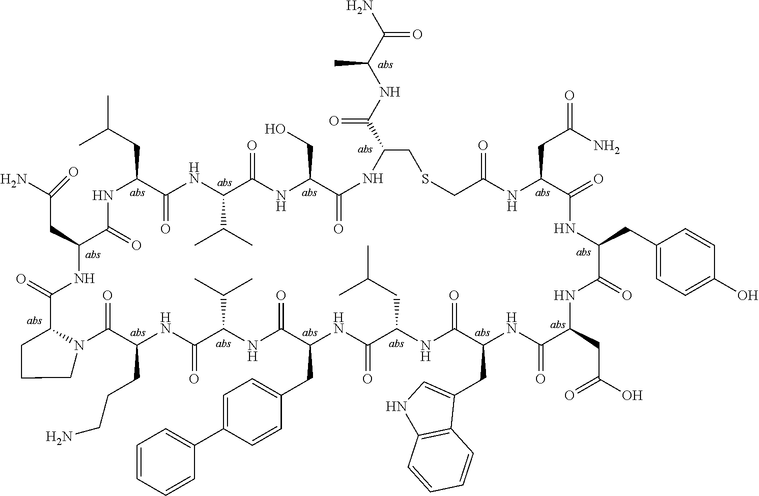 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)[C@H]2CCCN2C(=O)[C@H](CCCN)NC(=O)[C@H](C(C)C)NC(=O)[C@H](Cc2ccc(-c3ccccc3)cc2)NC1=O]

Example 1425 was prepared on a 50 μmol scale. The yield of the product was 20.4 mg, and its estimated purity by LCMS analysis was 95.3%. Analysis condition B: Retention time=1.39 min; ESI-MS(+) m/z [M+2H]²⁺: 935.9.

### Preparation of Example 1426

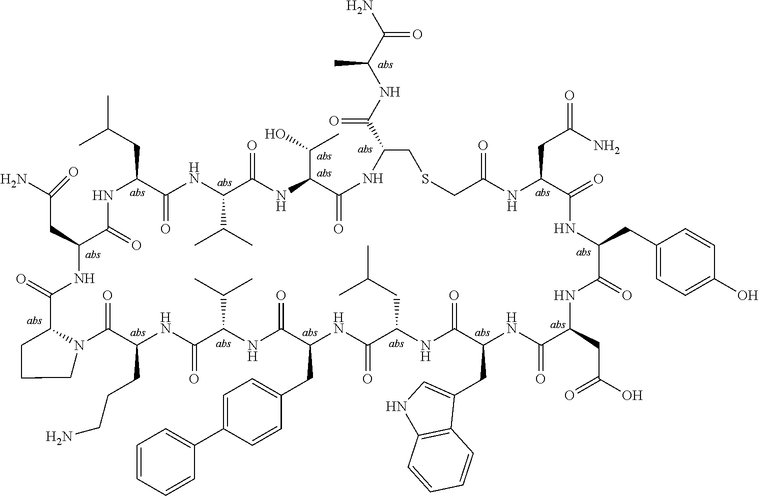 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CC(N)=O)NC(=O)[C@H]2CCCN2C(=O)[C@H](CCCN)NC(=O)[C@H](C(C)C)NC(=O)[C@H](Cc2ccc(-c3ccccc3)cc2)NC1=O]

Example 1426 was prepared on a 50 μmol scale. The yield of the product was 13.8 mg, and its estimated purity by LCMS analysis was 98.3%. Analysis condition B: Retention time=1.45 min; ESI-MS(+) m/z [M+2H]²⁺: 943.3.

### Preparation of Example 1427

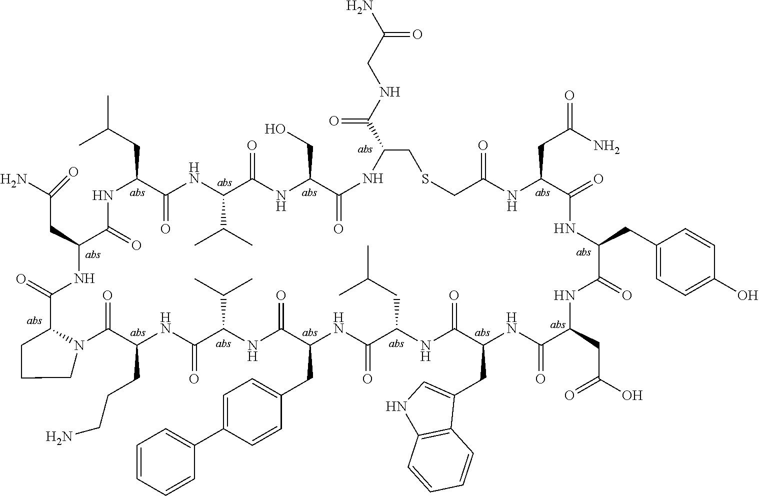 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CC(N)=O)NC(=O)[C@H]2CCCN2C(=O)[C@H](CCCN)NC(=O)[C@H](C(C)C)NC(=O)[C@H](Cc2ccc(-c3ccccc3)cc2)NC1=O]

Example 1428 was prepared on a 40 μmol scale. The yield of the product was 0.4 mg, and its estimated purity by LCMS analysis was 92.7%. Analysis condition A: Retention time=1.64 min; ESI-MS(+) m/z [M+2H]²⁺: 950.2.

### Preparation of Example 1428

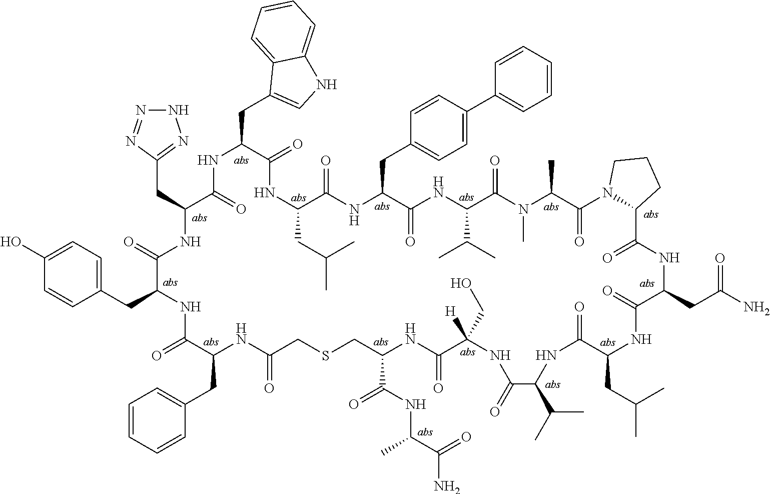 [SMILES: CC(C)C[C@@H]1NC(=O)[C@H](Cc2c[nH]c3ccccc23)NC(=O)[C@H](Cc2nn[nH]n2)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)[C@H]2CCCN2C(=O)[C@H](C)N(C)C(=O)[C@H](C(C)C)NC(=O)[C@H](Cc2ccc(-c3ccccc3)cc2)NC1=O]

Example 1428 was prepared on a 50 μmol scale. The yield of the product was 0.4 mg, and its estimated purity by LCMS analysis was 92.7%. Analysis condition A: Retention time=1.64 min; ESI-MS (+) m/z [M+2H]²⁺: 1010.2.

### Preparation of Example 1429

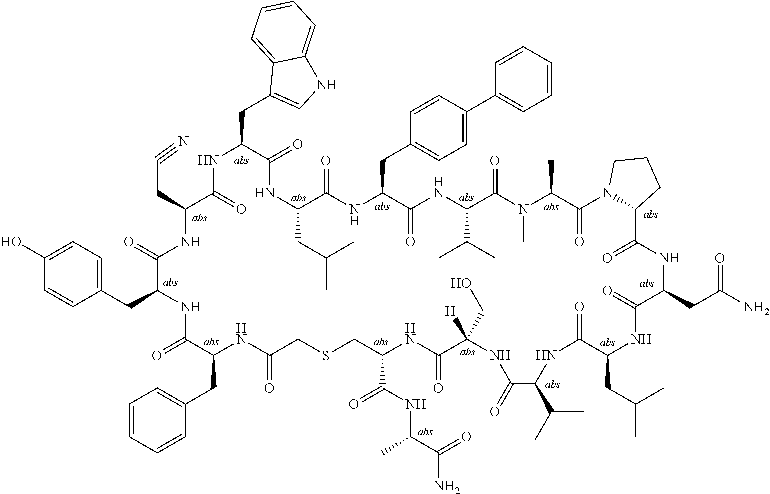 [SMILES: CC(C)C[C@@H]1NC(=O)[C@H](Cc2c[nH]c3ccccc23)NC(=O)[C@H](CC#N)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)[C@H]2CCCN2C(=O)[C@H](C)N(C)C(=O)[C@H](C(C)C)NC(=O)[C@H](Cc2ccc(-c3ccccc3)cc2)NC1=O]

Example 1429 was prepared on a 40 μmol scale. The yield of the product was 15.2 mg, and its estimated purity by LCMS analysis was 100%. Analysis condition A: Retention time=1.85 min; ESI-MS(+) m/z [M+H]⁺: 1855.

### Preparation of Example 1430

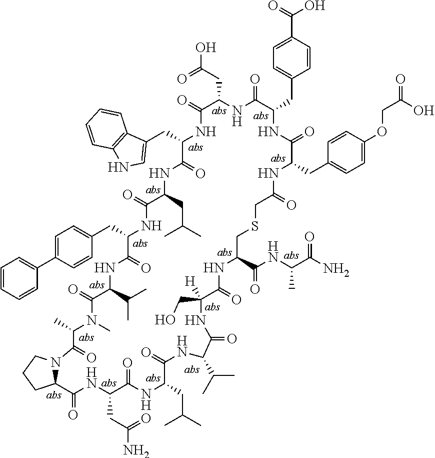 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)[C@H]2CCCN2C(=O)[C@H](C)N(C)C(=O)[C@H](C(C)C)NC(=O)[C@H](Cc2ccc(-c3ccccc3)cc2)NC1=O]

Example 1430 was prepared on a 50 μmol scale. The yield of the product was 38.4 mg, and its estimated purity by LCMS analysis was 100%. Analysis condition A: Retention time=1.38 min: ESI-MS(+) m/z [M+H]⁺: 1975.8.

### Preparation of Example 1431

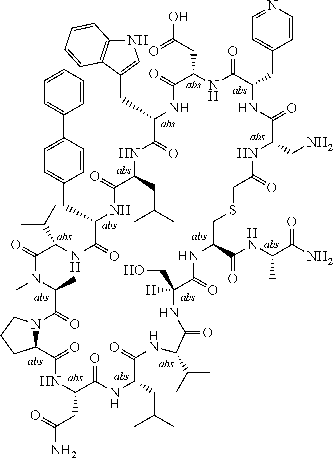 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](CN)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)[C@H]2CCCN2C(=O)[C@H](C)N(C)C(=O)[C@H](C(C)C)NC(=O)[C@H](Cc2ccc(-c3ccccc3)cc2)NC1=O]

Example 1431 was prepared on a 50 μmol scale. The yield of the product was 14.2 mg, and its estimated purity by LCMS analysis was 84.3%. Analysis condition A: Retention time=1.56 min; ESI-MS(+) m/z [M+H]⁺: 1816.1.

### Preparation of Example 1432

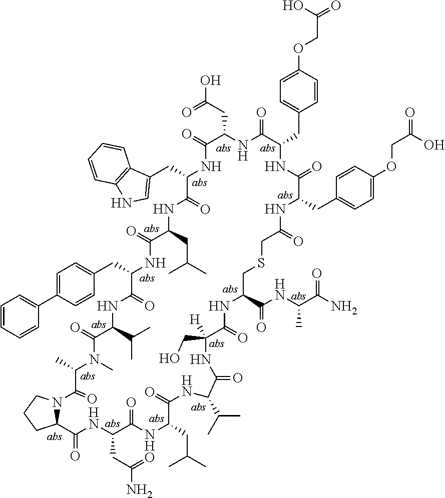 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)[C@H]2CCCN2C(=O)[C@H](C)N(C)C(=O)[C@H](C(C)C)NC(=O)[C@H](Cc2ccc(-c3ccccc3)cc2)NC1=O]

Example 1432 was prepared on a 50 μmol scale. The yield of the product was 18.8 mg, and its estimated purity by LCMS analysis was 100%. Analysis condition A: Retention time=1.21 min; ESI-MS(+) m/z [M+2H]²⁺: 1003.4.

### Preparation of Example 1433

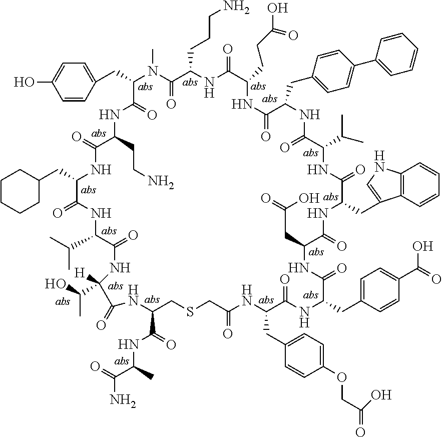 [SMILES: CC(C)[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@H](Cc2ccc(O)cc2)N(C)C(=O)[C@H](CCCN)NC(=O)[C@H](CCC(=O)O)NC(=O)[C@H](Cc2ccc(-c3ccccc3)cc2)NC1=O]

Example 1433 was prepared on a 50 μmol scale. The yield of the product was 20.2 mg, and its estimated purity by LCMS analysis was 90.6%. Analysis condition B: Retention time=1.55 min; ESI-MS(+) m/z [M+2H]²⁺: 1071.

### Preparation of Example 1434

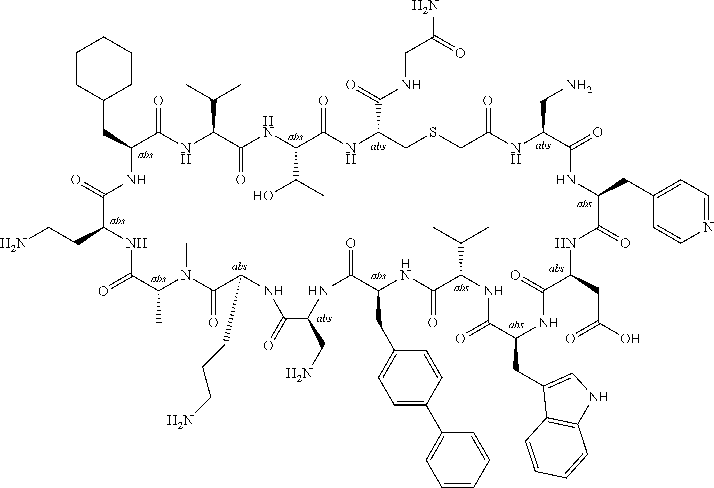 [SMILES: CC(C)[C@@H]1NC(=O)[C@H](Cc2c[nH]c3ccccc23)NC(=O)[C@H](CC(=O)O)NC(=O)[C@H](Cc2ccncc2)NC(=O)[C@H](CN)NC(=O)CSC[C@@H](C(=O)NCC(N)=O)NC(=O)[C@H](C(C)O)NC(=O)[C@H](C(C)C)NC(=O)[C@H](CC2CCCCC2)NC(=O)[C@H](CCN)NC(=O)[C@@H](C)N(C)C(=O)[C@H](CCCN)NC(=O)[C@H](CN)NC(=O)[C@H](Cc2ccc(-c3ccccc3)cc2)NC1=O]

Example 1434 was prepared on a 50 μmol scale. The yield of the product was 39 mg, and its estimated purity by LCMS analysis was 100%. Analysis condition A: Retention time=1.5 min; ESI-MS(+) m/z [M+H]⁺: 1815.3.

### Preparation of Example 1435

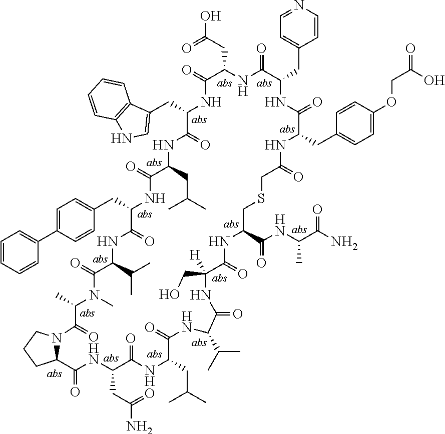 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)[C@H]2CCCN2C(=O)[C@H](C)N(C)C(=O)[C@H](C(C)C)NC(=O)[C@H](Cc2ccc(-c3ccccc3)cc2)NC1=O]

Example 1435 was prepared on a 50 μmol scale. The yield of the product was 22.5 mg, and its estimated purity by LCMS analysis was 87.4%. Analysis condition B: Retention time=1.53 min: ESI-MS(+) m/z [M+2H]²⁺: 967.1.

### Preparation of Example 1436

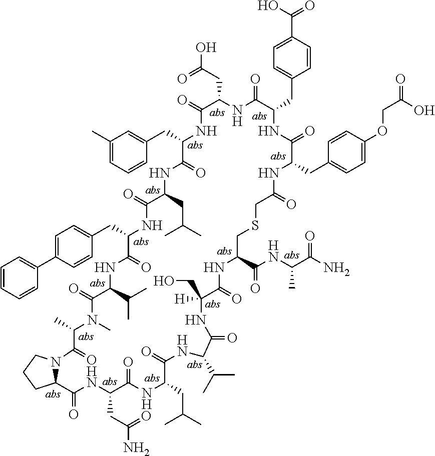 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)[C@H]3CCCN3C(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 1436 was prepared on a 50 μmol scale. The yield of the product was 7 mg, and its estimated purity by LCMS analysis was 100%. Analysis condition A: Retention time=1.3 min; ESI-MS(+) m/z [M+2H]²⁺: 976.1.

### Preparation of Example 1437

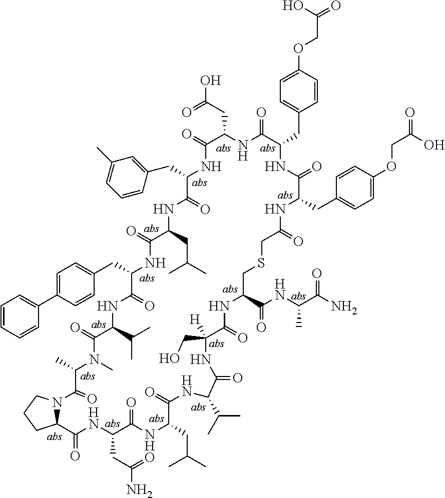 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)[C@H]3CCCN3C(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 1437 was prepared on a 50 μmol scale. The yield of the product was 13.1 mg, and its estimated purity by LCMS analysis was 98.9%. Analysis condition B: Retention time=1.63 min: ESI-MS(+) m/z [M+3H]³⁺: 661.1.

### Preparation of Example 1438

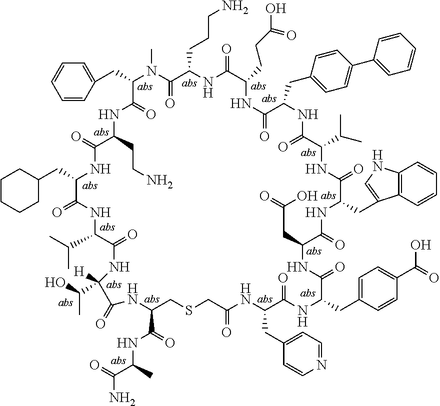 [SMILES: CC(C)[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@H](Cc2ccccc2)N(C)C(=O)[C@H](CCCN)NC(=O)[C@H](CCC(=O)O)NC(=O)[C@H](Cc2ccc(-c3ccccc3)cc2)NC1=O]

Example 1438 was prepared on a 50 μmol scale. The yield of the product was 28.5 mg, and its estimated purity by LCMS analysis was 94.4%. Analysis condition A: Retention time=1.48 min: ESI-MS(+) m/z [M+2H]²⁺: 1026.9.

### Preparation of Example 1439

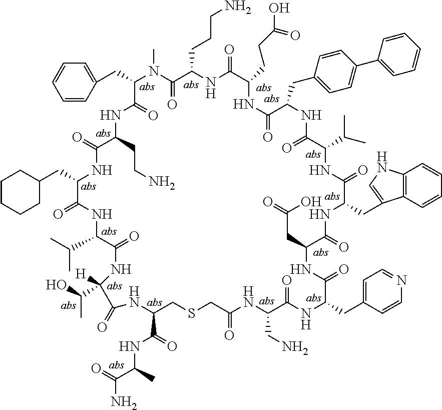 [SMILES: CC(C)[C@@H]1NC(=O)[C@H](Cc2c[nH]c3ccccc23)NC(=O)[C@H](CC(=O)O)NC(=O)[C@H](Cc2ccncc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@H](Cc2ccccc2)N(C)C(=O)[C@H](CCCN)NC(=O)[C@H](CCC(=O)O)NC(=O)[C@H](Cc2ccc(-c3ccccc3)cc2)NC1=O]

Example 1439 was prepared on a 50 μmol scale. The yield of the product was 12.2 mg, and its estimated purity by LCMS analysis was 94.8%. Analysis condition B: Retention time=1.45 min; ESI-MS(+) m/z [M+H]⁺: 1947.

### Preparation of Example 1440

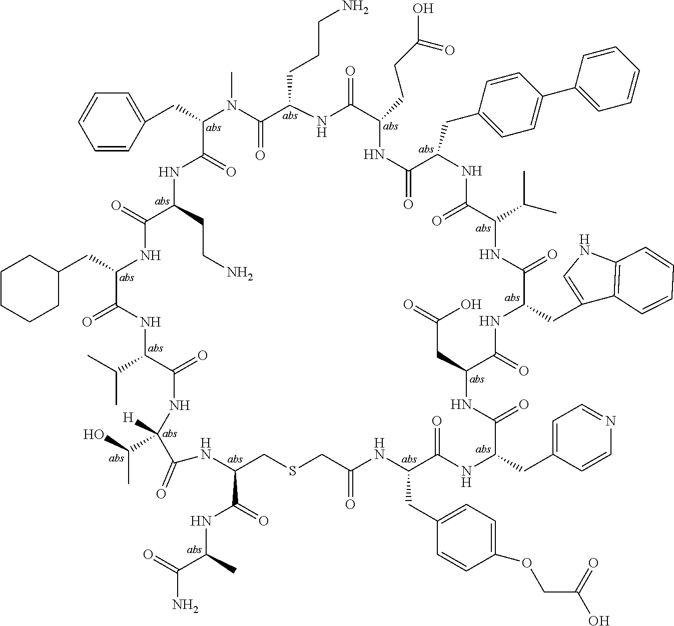

Example 1440 was prepared on a 50 μmol scale. The yield of the product was 12 mg, and its estimated purity by LCMS analysis was 100%. Analysis condition A: Retention time=1.46, 1.55 min; ESI-MS(+) m/z [M+2H]²⁺: 1041.94, 1041.96.

### Preparation of Example 1441

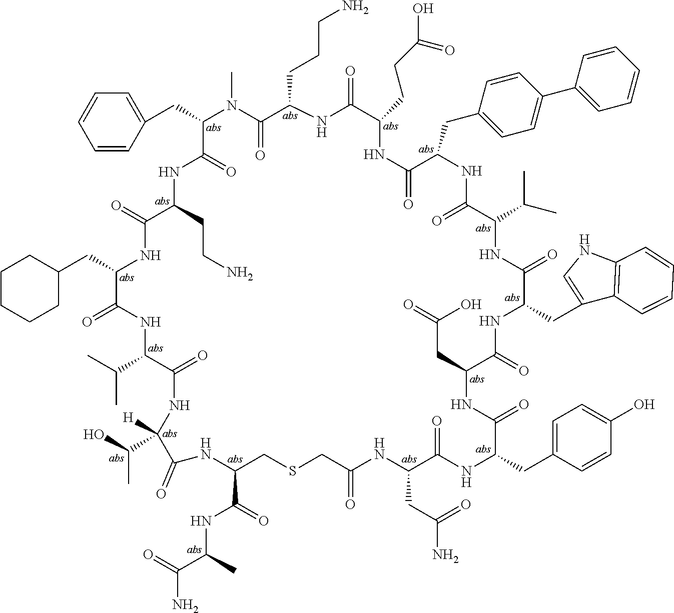

Example 1441 was prepared on a 50 μmol scale. The yield of the product was 18 mg, and its estimated purity by LCMS analysis was 94.7%. Analysis condition B: Retention time=1.6 min; ESI-MS(+) m/z [M+H]⁺: 1990.

### Preparation of Example 1442

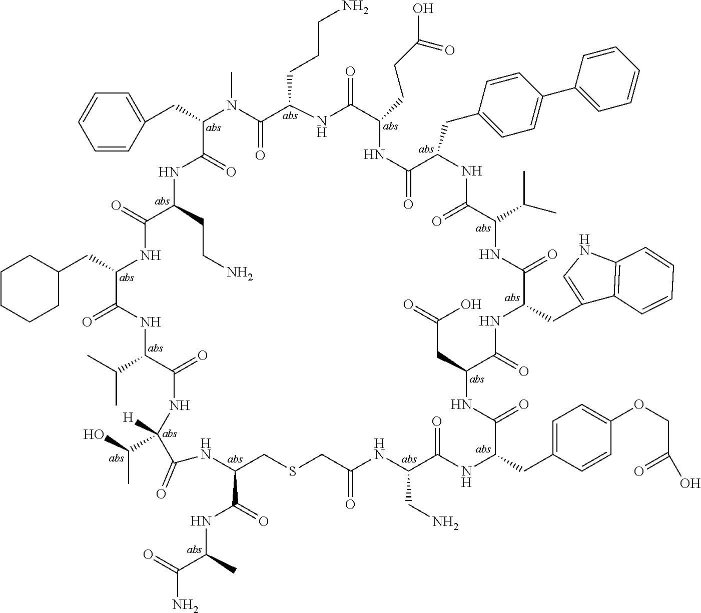

Example 1442 was prepared on a 50 μmol scale. The yield of the product was 31.5 mg, and its estimated purity by LCMS analysis was 90.9%. Analysis condition B: Retention time=1.48 min; ESI-MS(+) m/z [M+2H]²⁺: 1010.9.

### Preparation of Example 1443

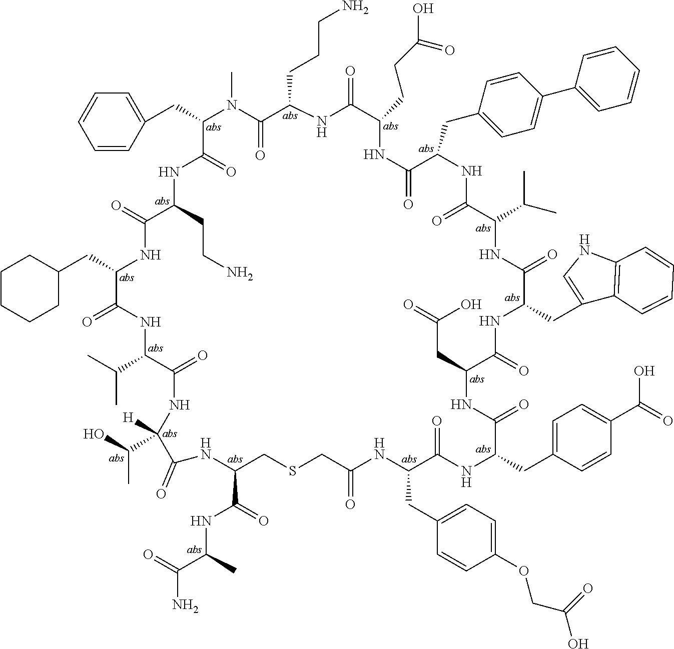

Example 1443 was prepared on a 50 μmol scale. The yield of the product was 14.3 mg, and its estimated purity by LCMS analysis was 95.4%. Analysis condition B: Retention time=1.56 min; ESI-MS(+) m/z [M+2H]²⁺: 1063.2.

### Preparation of Example 1444

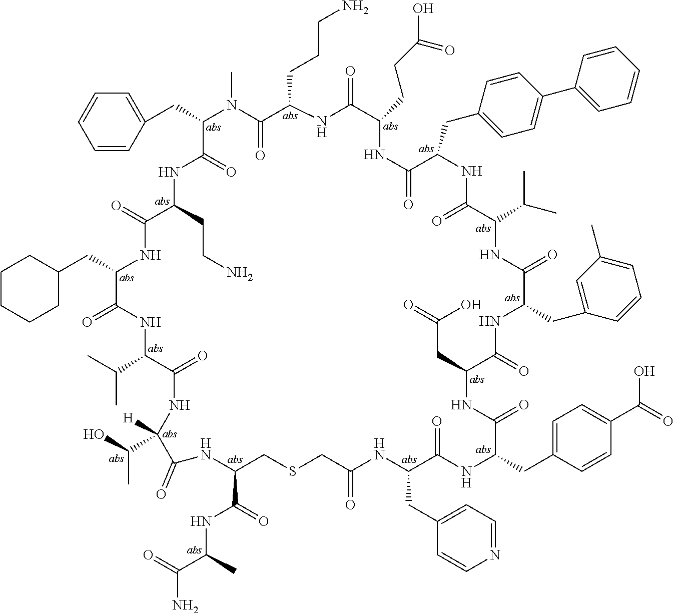

Example 1444 was prepared on a 50 μmol scale. The yield of the product was 17.7 mg, and its estimated purity by LCMS analysis was 94.9%. Analysis condition A: Retention time=1.52 min; ESI-MS(+) m/z [M+2H]²⁺: 1013.9.

### Preparation of Example 1445

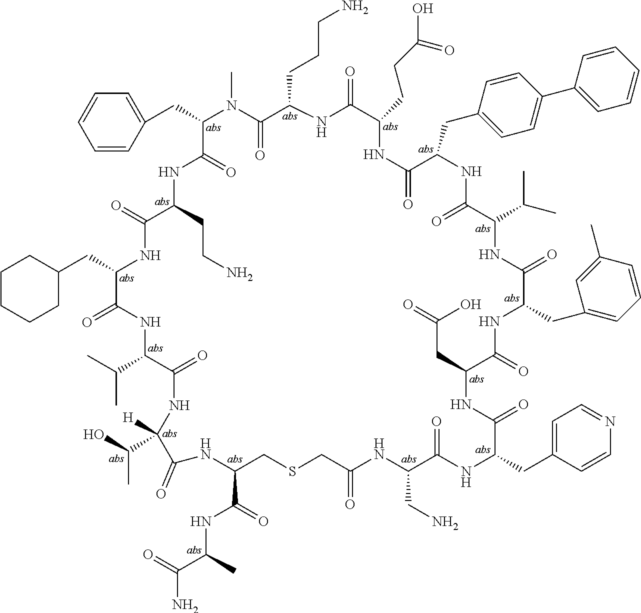

Example 1445 was prepared on a 50 μmol scale. The yield of the product was 26.3 mg, and its estimated purity by LCMS analysis was 99.4%. Analysis condition B: Retention time=1.5 min ESI-MS(+) m/z [M+H]⁺: 1921.7.

### Preparation of Example 1446

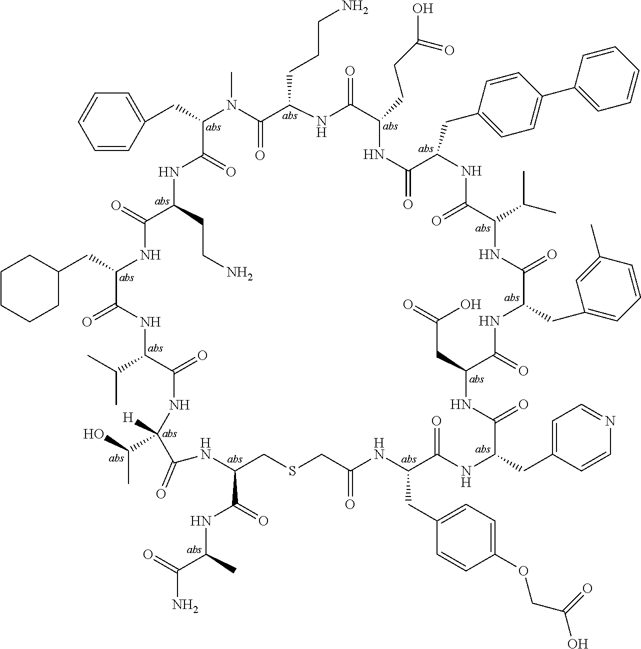

Example 1446 was prepared on a 50 μmol scale. The yield of the product was 14.7 mg, and its estimated purity by LCMS analysis was 92.7%. Analysis condition B: Retention time=1.5 min; ESI-MS(+) m/z [M+2H]²⁺: 1029.3.

### Preparation of Example 1447

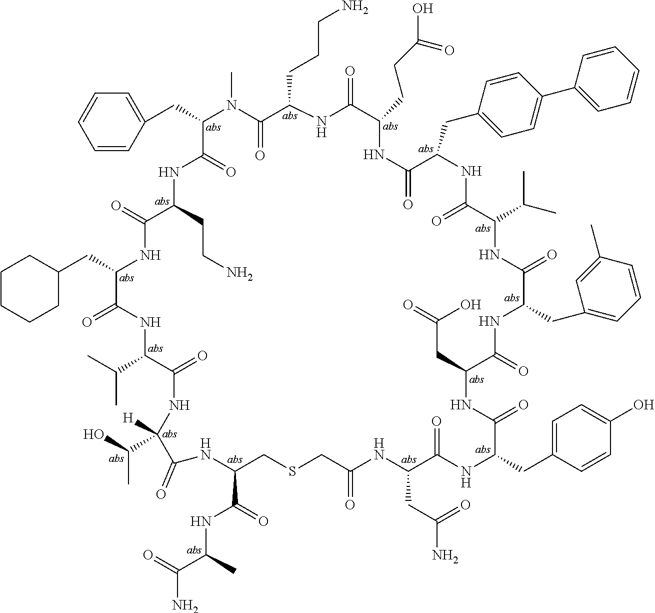

Example 1447 was prepared on a 50 μmol scale. The yield of the product was 20 mg, and its estimated purity by LCMS analysis was 96%. Analysis condition B: Retention time=1.58 min: ESI-MS(+) m/z [M+H]⁺: 1965.2.

### Preparation of Example 1448

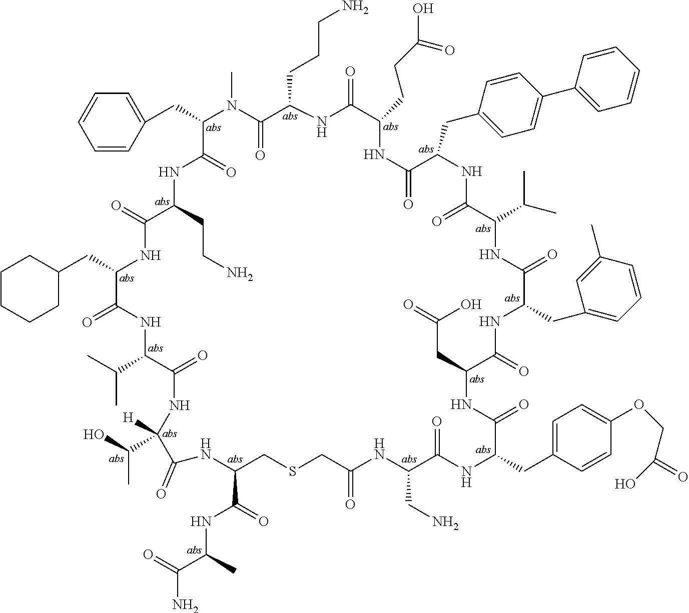

Example 1448 was prepared on a 50 μmol scale. The yield of the product was 15.1 mg, and its estimated purity by LCMS analysis was 90.2%. Analysis condition B: Retention time=1.5 min: ESI-MS(+) m/z [M+3H]³⁺: 666.2.

### Preparation of Example 1449

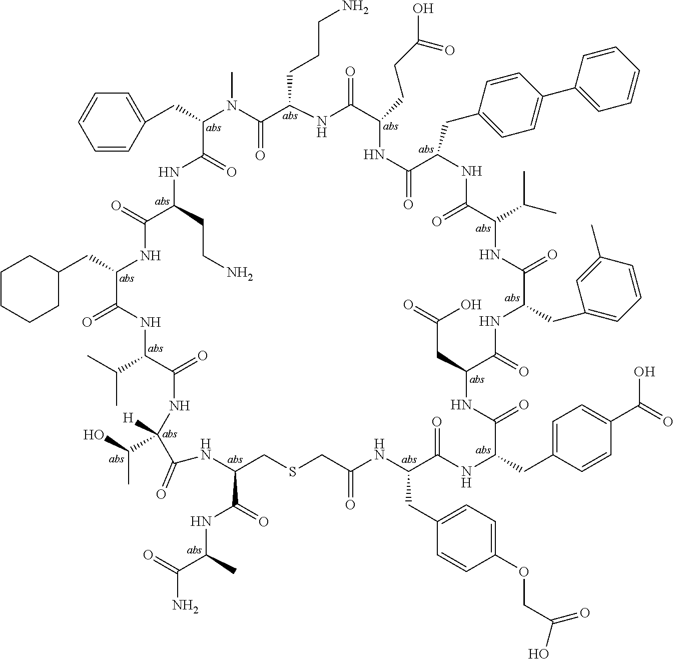

Example 1449 was prepared on a 50 μmol scale. The yield of the product was 18.4 mg, and its estimated purity by LCMS analysis was 1000%. Analysis condition A: Retention time=1.36 min ESI-MS(+) m/z [M+2H]⁺: 1051.1.

### Preparation of Example 1450

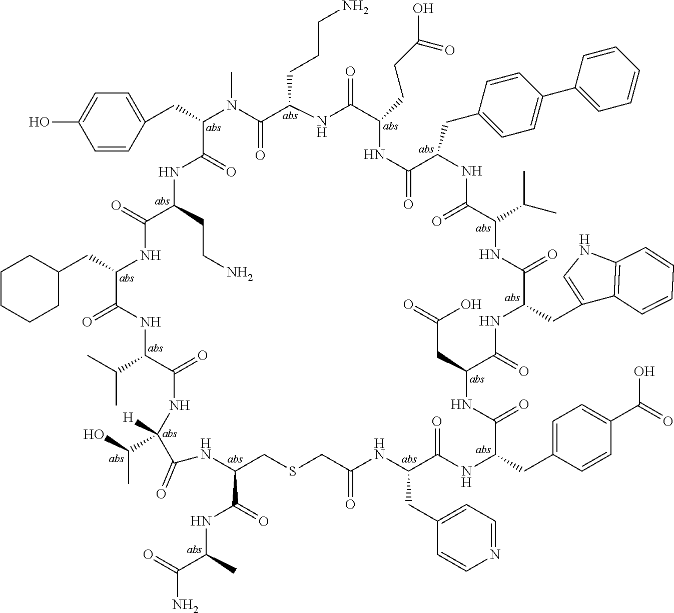

Example 1450 was prepared on a 50 μmol scale. The yield of the product was 16.1 mg, and its estimated purity by LCMS analysis was 94.7%. Analysis condition A: Retention time=1.43 min; ESI-MS(+) m/z [M+2H]^{Z+}: 1035.

### Preparation of Example 1451

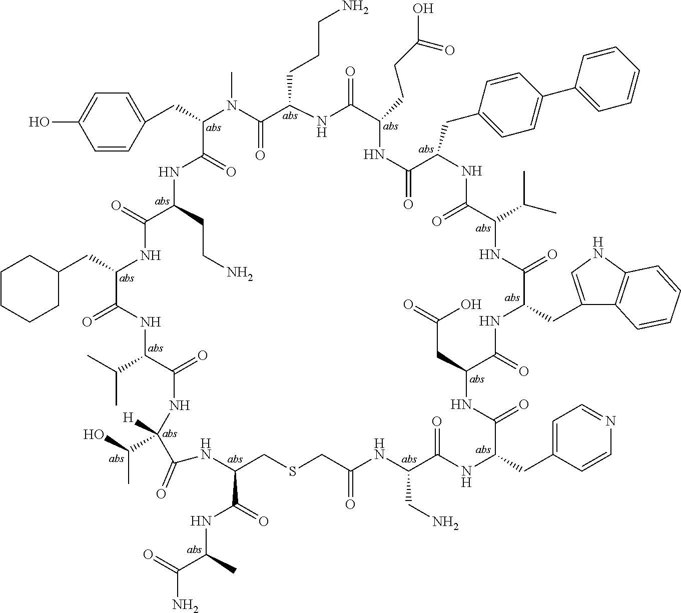

Example 1451 was prepared on a 50 μmol scale. The yield of the product was 18.2 mg, and its estimated purity by LCMS analysis was 93.3%. Analysis condition B: Retention time=1.3 min; ESI-MS(+) m/z [M+H]⁺: 1963.

### Preparation of Example 1452

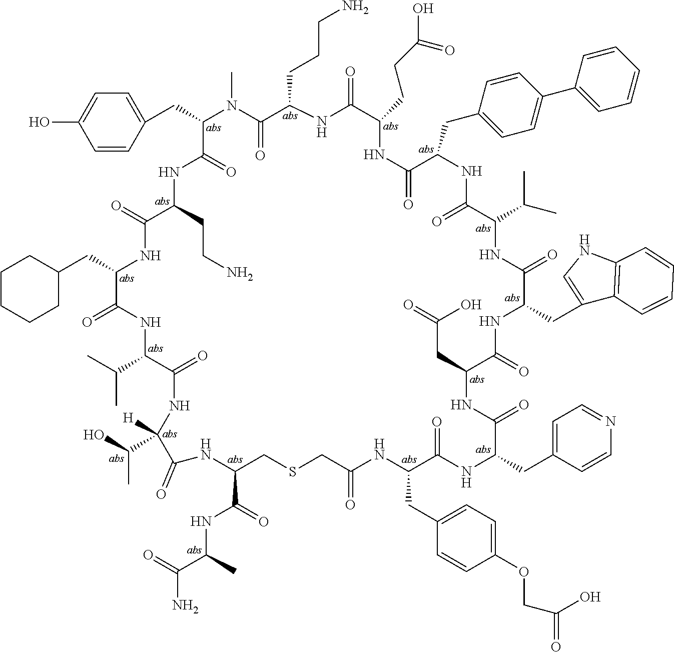

Example 1452 was prepared on a 50 μmol scale. The yield of the product was 17.2 mg, and its estimated purity by LCMS analysis was 95.3%. Analysis condition B: Retention time=1.38 min: ESI-MS(+) m/z [M+2H]²⁺: 1050.1.

### Preparation of Example 1453

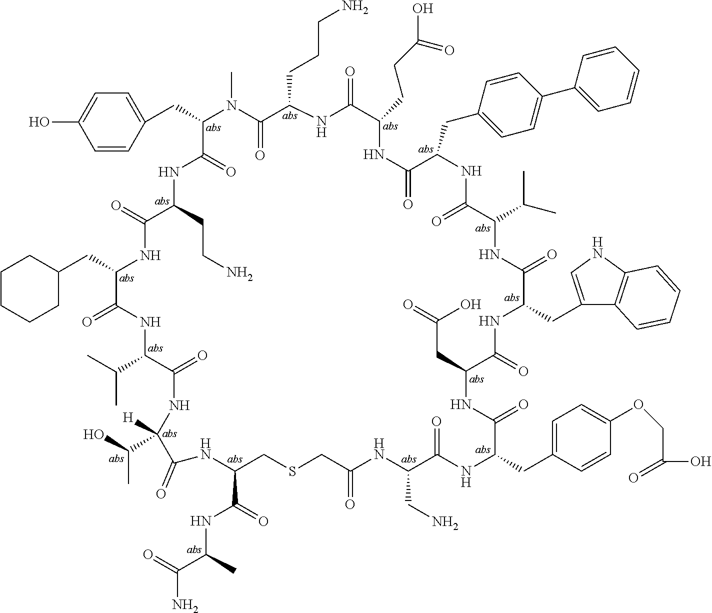

Example 1453 was prepared on a 50 μmol scale. The yield of the product was 11 mg, and its estimated purity by LCMS analysis was 83.3%. Analysis condition B: Retention time=1.39 min: ESI-MS(+) m/z [M+3H]⁺: 679.2.

### Preparation of Example 1501

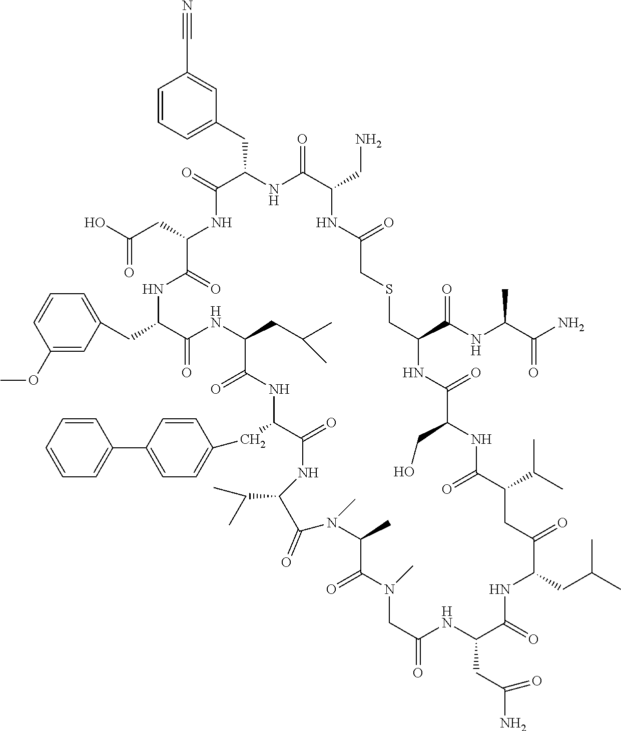 [SMILES: COc1cccc(C[C@@H]2NC(=O)[C@H](CC(=O)O)NC(=O)[C@H](Cc3cccc(C#N)c3)NC(=O)[C@H](CN)NC(=O)CSC[C@@H](C(=O)N[C@@H](C)C(N)=O)NC(=O)[C@H](CO)NC(=O)[C@H](C(C)C)CC(=O)[C@H](CC(C)C)NC(=O)[C@H](CC(N)=O)NC(=O)CN(C)C(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 1501 was prepared on a 40 μmol scale. The yield of the product was 5.4 mg, and its estimated purity by LCMS analysis was 91%. Analysis condition 2: Retention time=1.6 min: ESI-MS(+) m/z (M+3H)³⁺: 894.1.

### Biological Activity

The ability of the compounds of formula (I) to bind to PD-1 was investigated using a Jurkat-PD-1 Cell Binding High-Content Screening Assay.

### Jurkat-PD-1 Cell Binding High-Content Screening Assay (CBA)

Phycoerythrin (PE) was covalently linked to the Ig epitope tag of human PD-L1-Ig and fluorescently-labeled PD-L1-Ig was used for binding studies with a Jurkat cell line over-expressing human PD-1 (Jurkat-PD-1). Briefly, 8×10³ Jurkat-hPD-1 cells were seeded into 384 well plates in 20 μL of DMEM supplemented with 10% fetal calf serum. 100 nL of compound was added to cells followed by incubation at 37° C. for 2 hours. Then, 5 μL of PE-labeled PD-L1-Ig (20 nM final), diluted in DMEM supplemented with 10% fetal calf serum. After 1 hour incubation, cells were fixed with 4% paraformaldehyde in PBS containing 10 μg/mL Hoechst 33342 and then washed 3× in 100 μL PBS. Data was collected and processed using a Cell Insight NXT High Content Imager and associated software.

### Protein Sequence Information

```
hPDL1(18-239)-TVMV-mIgG1(221-447)-C225S
    1 AFTVTVPKDL YVVEYGSNMT IECKFPVEKQ
        LDLAALIVYW EMEDKNIIQF
  51 VHGEEDLKVQ HSSYRQRARL LKDQLSLGNA
        ALQITDVKLQ DAGVYRCMIS
101 YGGADYKRIT VKVNAPYNKI NQRILVVDPV
        TSEHELTCQA EGYPKAEVIW
151 TSSDHQVLSG KTTTTNSKRE EKLFNVTSTL
        RINTTTNEIF YCTFRRLDPE
201 ENHTAEL VIP ELPLAHPPNE RTGSPGGGGG
        RETVRFQGGT GDAVPRDSGC
251 KPCICTVPEV SSVFIFPPKP KDVLTITLTP
        KVTCVVVDIS KDDPEVQFSW
301 FVDDVEVHTA QTQPREEQFN STFRSVSELP
        IMHQDWLNGK EFKCRVNSAA
351 FPAPIEKTIS KTKGRPKAPQ VYTIPPPKEQ
        MAKDKVSLTC MITDFFPEDI
401 TVEWQWNGQP AENYKNTQPI MDTDGSYFVY
        SKLNVQKSNW EAGNTFTCSV
451 LHEGLHNHHT EKSLSHSPGK
```

Table 3 lists the IC₅₀ values for representative examples of this disclosure measured in the Jurkat hPD1-PDL1 assay.

TABLE 3

| Example | Jurkat HPD1-PDL1 |
|---|---|
| Number | IC50 (μM) |
| 1000 | 0.26 |
| 1001 | 0.92 |
| 1002 | 0.79 |
| 1003 | 0.76 |
| 1004 | 0.27 |
| 1005 | 0.03 |
| 1006 | 0.04 |
| 1007 | 0.15 |
| 1008 | 0.17 |
| 1009 | 0.26 |
| 1010 | 0.26 |
| 1011 | 0.31 |
| 1012 | 0.10 |
| 1013 | 0.11 |
| 1014 | 0.91 |
| 1015 | 0.03 |
| 1016 | 0.03 |
| 1017 | 0.04 |
| 1018 | 0.04 |
| 1019 | 0.04 |
| 1020 | 0.04 |
| 1021 | 0.04 |
| 1022 | 0.01 |
| 1023 | 0.04 |
| 1024 | 0.04 |
| 1025 | 0.04 |
| 1026 | 0.04 |
| 1027 | 0.05 |
| 1028 | 0.05 |
| 1029 | 0.05 |
| 1030 | 0.06 |
| 1031 | 0.06 |
| 1032 | 0.06 |
| 1033 | 0.06 |
| 1034 | 0.06 |
| 1035 | 0.06 |
| 1036 | 0.06 |
| 1037 | 0.07 |
| 1038 | 0.07 |
| 1039 | 0.07 |
| 1040 | 0.07 |
| 1041 | 0.07 |
| 1042 | 0.07 |
| 1043 | 0.08 |
| 1044 | 0.08 |
| 1045 | 0.08 |
| 1046 | 0.08 |
| 1047 | 0.09 |
| 1048 | 0.09 |
| 1049 | 0.09 |
| 1050 | 0.11 |
| 1051 | 0.11 |
| 1052 | 0.12 |
| 1053 | 0.12 |
| 1054 | 0.12 |
| 1055 | 0.12 |
| 1056 | 0.13 |
| 1057 | 0.13 |
| 1058 | 0.13 |
| 1059 | 0.14 |
| 1060 | 0.14 |
| 1061 | 0.14 |
| 1062 | 0.16 |
| 1063 | 0.16 |
| 1064 | 0.16 |
| 1065 | 0.12 |
| 1066 | 0.16 |
| 1067 | 0.16 |
| 1068 | 0.17 |
| 1069 | 0.16 |
| 1070 | 0.18 |
| 1071 | 0.19 |
| 1072 | 0.19 |
| 1073 | 0.19 |
| 1074 | 0.19 |
| 1075 | 0.20 |
| 1076 | 0.21 |
| 1077 | 0.21 |
| 1078 | 0.22 |
| 1079 | 0.22 |
| 1080 | 0.23 |
| 1081 | 0.23 |
| 1082 | 0.23 |
| 1083 | 0.24 |
| 1084 | 0.24 |
| 1085 | 0.24 |
| 1086 | 0.25 |
| 1087 | 0.25 |
| 1088 | 0.25 |
| 1089 | 0.25 |
| 1090 | 0.43 |
| 1091 | 0.26 |
| 1092 | 0.26 |
| 1093 | 0.27 |
| 1094 | 0.28 |
| 1095 | 0.28 |
| 1096 | 0.28 |
| 1097 | 0.28 |
| 1098 | 0.31 |
| 1099 | 0.31 |
| 1100 | 0.31 |
| 1101 | 0.30 |
| 1102 | 0.31 |
| 1103 | 0.31 |
| 1104 | 0.32 |
| 1105 | 0.33 |
| 1106 | 0.33 |
| 1107 | 0.34 |
| 1108 | 0.34 |
| 1109 | 0.35 |
| 1110 | 0.35 |
| 1111 | 0.35 |
| 1112 | 0.37 |
| 1113 | 0.37 |
| 1114 | 0.39 |
| 1115 | 0.40 |
| 1116 | 0.40 |
| 1117 | 0.41 |
| 1118 | 0.42 |
| 1119 | 0.42 |
| 1120 | 0.43 |
| 1121 | 0.43 |
| 1122 | 0.43 |
| 1123 | 0.44 |
| 1124 | 0.45 |
| 1125 | 0.47 |
| 1126 | 0.48 |
| 1127 | 0.48 |
| 1128 | 0.49 |
| 1129 | 0.50 |
| 1130 | 0.50 |
| 1131 | 0.53 |
| 1132 | 0.53 |
| 1133 | 0.54 |
| 1134 | 0.54 |
| 1135 | 0.55 |
| 1136 | 0.56 |
| 1137 | 0.56 |
| 1138 | 0.57 |
| 1139 | 0.59 |
| 1140 | 0.60 |
| 1141 | 0.61 |
| 1142 | 0.61 |
| 1143 | 0.62 |
| 1144 | 0.63 |
| 1145 | 0.64 |
| 1146 | 0.65 |
| 1147 | 0.66 |
| 1148 | 0.67 |
| 1149 | 0.69 |
| 1150 | 0.69 |
| 1151 | 0.70 |
| 1152 | 0.74 |
| 1153 | 0.76 |
| 1154 | 0.79 |
| 1155 | 0.81 |
| 1156 | 0.82 |
| 1157 | 0.83 |
| 1158 | 0.84 |
| 1159 | 0.86 |
| 1160 | 0.87 |
| 1161 | 0.87 |
| 1162 | 0.90 |
| 1163 | 0.90 |
| 1164 | 0.90 |
| 1165 | 0.90 |
| 1166 | 0.90 |
| 1167 | 0.91 |
| 1168 | 0.93 |
| 1169 | 0.93 |
| 1170 | 0.94 |
| 1171 | 0.94 |
| 1172 | 0.95 |
| 1173 | 0.95 |
| 1174 | 0.97 |
| 1175 | 0.50 |
| 1177 | 0.24 |
| 1178 | 0.07 |
| 1179 | 0.08 |
| 1180 | 0.02 |
| 1181 | 0.10 |
| 1182 | 0.24 |
| 1183 | 0.04 |
| 1184 | 0.02 |
| 1185 | 0.03 |
| 1186 | 0.38 |
| 1187 | 0.29 |
| 1188 | 0.05 |
| 1189 | 0.07 |
| 1190 | 0.55 |
| 1191 | 0.69 |
| 1192 | 0.92 |
| 1193 | 0.04 |
| 1194 | 0.04 |
| 1195 | 0.37 |
| 1196 | 0.95 |
| 1197 | 0.42 |
| 1198 | 0.76 |
| 1199 | 0.10 |
| 1200 | 0.03 |
| 1201 | 0.01 |
| 1202 | 0.08 |
| 1203 | 0.85 |
| 1204 | 0.40 |
| 1205 | 0.44 |
| 1206 | 0.45 |
| 1207 | 0.32 |
| 1208 | 0.82 |
| 1209 | 0.78 |
| 1210 | 0.60 |
| 1211 | 0.65 |
| 1212 | 0.61 |
| 1213 | 0.59 |
| 1214 | 0.06 |
| 1215 | 0.37 |
| 1216 | 0.09 |
| 1217 | 0.08 |
| 1218 | 0.23 |
| 1219 | 0.61 |
| 1220 | 0.72 |
| 1221 | 0.50 |
| 1222 | 0.06 |
| 1223 | 0.99 |
| 1224 | 0.16 |
| 1225 | 0.96 |
| 1226 | 0.89 |
| 1227 | 0.65 |
| 1228 | 0.72 |
| 1229 | 0.44 |
| 1230 | 0.06 |
| 1231 | 0.90 |
| 1232 | 0.33 |
| 1233 | 0.20 |
| 1234 | 0.29 |
| 1235 | 0.11 |
| 1236 | 0.44 |
| 1237 | 0.52 |
| 1238 | 0.17 |
| 1239 | 0.11 |
| 1240 | 0.17 |
| 1241 | 0.27 |
| 1242 | 0.24 |
| 1243 | 0.06 |
| 1244 | 0.19 |
| 1245 | 0.11 |
| 1246 | 0.25 |
| 1247 | 0.23 |
| 1248 | 0.36 |
| 1249 | 0.27 |
| 1250 | 0.32 |
| 1251 | 0.36 |
| 1252 | 0.19 |
| 1253 | 0.71 |
| 1254 | 0.85 |
| 1255 | 0.43 |
| 1256 | 0.34 |
| 1257 | 0.08 |
| 1258 | 0.24 |
| 1259 | 0.26 |
| 1260 | 0.47 |
| 1261 | 0.11 |
| 1262 | 0.11 |
| 1263 | 0.46 |
| 1264 | 0.16 |
| 1265 | 0.10 |
| 1266 | 0.34 |
| 1267 | 0.19 |
| 1268 | 0.43 |
| 1269 | 0.20 |
| 1270 | 0.21 |
| 1271 | 0.55 |
| 1272 | 0.12 |
| 1273 | 0.48 |
| 1274 | 0.08 |
| 1275 | 0.03 |
| 1276 | 0.84 |
| 1277 | 0.14 |
| 1278 | 0.31 |
| 1279 | 0.32 |
| 1280 | 0.50 |
| 1281 | 0.46 |
| 1282 | 0.29 |
| 1283 | 0.70 |
| 1284 | 0.82 |
| 1285 | 0.31 |
| 1286 | 0.75 |
| 1287 | 0.13 |
| 1288 | 0.08 |
| 1289 | 0.17 |
| 1290 | 0.14 |
| 1291 | 0.09 |
| 1292 | 0.09 |
| 1293 | 0.10 |
| 1294 | 0.14 |
| 1295 | 0.21 |
| 1296 | 0.14 |
| 1297 | 0.42 |
| 1298 | 0.06 |
| 1299 | 0.07 |
| 1300 | 0.12 |
| 1301 | 0.15 |
| 1302 | 0.35 |
| 1303 | 0.07 |
| 1304 | 0.06 |
| 1305 | 0.06 |
| 1306 | 0.46 |
| 1307 | 0.17 |
| 1308 | 0.41 |
| 1309 | 0.34 |
| 1310 | 0.34 |
| 1311 | 0.75 |
| 1312 | 0.76 |
| 1313 | 0.24 |
| 1314 | 0.09 |
| 1315 | 0.53 |
| 1316 | 0.08 |
| 1317 | 0.06 |
| 1318 | 0.38 |
| 1319 | 0.13 |
| 1320 | 0.15 |
| 1321 | 0.11 |
| 1322 | 0.12 |
| 1323 | 0.51 |
| 1324 | 0.27 |
| 1325 | 0.59 |
| 1326 | 0.15 |
| 1327 | 0.12 |
| 1328 | 0.48 |
| 1329 | 0.26 |
| 1330 | 0.39 |
| 1331 | 0.08 |
| 1332 | 0.79 |
| 1333 | 0.68 |
| 1334 | 0.43 |
| 1335 | 0.14 |
| 1336 | 0.41 |
| 1337 | 0.87 |
| 1338 | 0.11 |
| 1339 | 0.75 |
| 1340 | 0.33 |
| 1341 | 0.20 |
| 1342 | 0.07 |
| 1343 | 0.37 |
| 1344 | 0.22 |
| 1345 | 0.12 |
| 1346 | 0.31 |
| 1347 | 0.16 |
| 1348 | 0.22 |
| 1349 | 0.33 |
| 1350 | 0.21 |
| 1351 | 0.38 |
| 1352 | 0.42 |
| 1353 | 0.09 |
| 1354 | 0.21 |
| 1355 | 0.41 |
| 1356 | 0.27 |
| 1357 | 0.21 |
| 1358 | 0.40 |
| 1359 | 0.27 |
| 1360 | 0.57 |
| 1361 | 0.38 |
| 1362 | 0.19 |
| 1363 | 0.94 |
| 1364 | 0.59 |
| 1365 | 0.76 |
| 1366 | 0.57 |
| 1367 | 0.31 |
| 1368 | 0.26 |
| 1369 | 0.33 |
| 1370 | 0.76 |
| 1371 | 0.23 |
| 1372 | 0.69 |
| 1373 | 0.18 |
| 1374 | 0.01 |
| 1375 | 0.08 |
| 1376 | 0.07 |
| 1377 | 0.04 |
| 1378 | 0.11 |
| 1379 | 0.04 |
| 1380 | 0.07 |
| 1381 | 0.16 |
| 1382 | 0.13 |
| 1383 | 0.63 |
| 1384 | 0.22 |
| 1385 | 0.28 |
| 1386 | 0.14 |
| 1387 | 0.01 |
| 1388 | 0.02 |
| 1389 | 0.05 |
| 1390 | 0.02 |
| 1391 | 0.04 |
| 1392 | 0.05 |
| 1393 | 0.02 |
| 1394 | 0.09 |
| 1395 | 0.16 |
| 1396 | 0.07 |
| 1397 | 0.04 |
| 1398 | 0.05 |
| 1399 | 0.07 |
| 1400 | 0.17 |
| 1401 | 0.17 |
| 1402 | 0.26 |
| 1403 | 0.07 |
| 1404 | 0.39 |
| 1405 | 0.66 |
| 1406 | 0.35 |
| 1407 | 0.28 |
| 1408 | 0.09 |
| 1409 | 0.28 |
| 1410 | 0.14 |
| 1411 | 0.44 |
| 1412 | 0.48 |
| 1413 | 0.09 |
| 1414 | 0.32 |
| 1415 | 0.08 |
| 1416 | 0.24 |
| 1417 | 0.07 |
| 1418 | 0.21 |
| 1419 | 0.12 |
| 1420 | 0.13 |
| 1421 | 0.06 |
| 1422 | 0.14 |
| 1423 | 0.10 |
| 1424 | 0.66 |
| 1425 | 0.19 |
| 1426 | 0.22 |
| 1427 | 0.12 |
| 1428 | 0.37 |
| 1429 | 0.01 |
| 1430 | 0.08 |
| 1431 | 0.26 |
| 1432 | 0.08 |
| 1433 | 0.26 |
| 1434 | 0.10 |
| 1435 | 0.09 |
| 1436 | 0.04 |
| 1437 | 0.03 |
| 1438 | 0.07 |
| 1439 | 0.09 |
| 1440 | 0.07 |
| 1441 | 0.06 |
| 1442 | 0.07 |
| 1443 | 0.08 |
| 1444 | 0.05 |
| 1445 | 0.01 |
| 1446 | 0.02 |
| 1447 | 0.03 |
| 1448 | 0.03 |
| 1449 | 0.03 |
| 1450 | 0.21 |
| 1451 | 0.52 |
| 1452 | 0.14 |
| 1453 | 0.32 |
| 1501 | 0.01 |

The compounds of formula (I) possess activity as inhibitors of the PD-1/PD-L1 interaction, and therefore, can be used in the treatment of diseases or deficiencies associated with the PD-1/PD-L1 interaction. Via inhibition of the PD-1/PD-L1 interaction, the compounds of the present disclosure can be employed to treat infectious diseases such as HIV, septic shock, Hepatitis A, B, C, or D and cancer.

It is to be appreciated that the Detailed Description section, and not the Summary and Abstract sections, is intended to be used to interpret the claims. The Summary and Abstract sections can set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

What is claimed is:

1. A compound of formula (I) 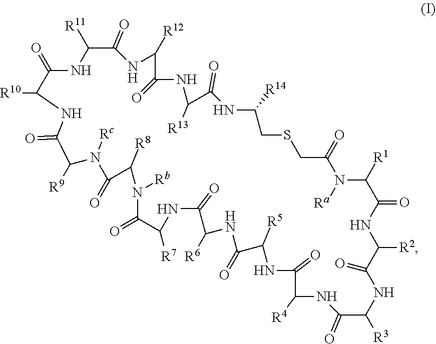
or a pharmaceutically acceptable salt thereof, wherein:
R¹ is selected from hydrogen, C₁-C₆alkyl, amidoC₁-C₆alkyl, aminoC₁-C₆alkyl, arylC₁-C₆alkyl, carboxyC₁-C₆alkyl, (C₃-C₈cycloalkyl)C₁-C₆alkyl, heteroarylC₁-C₆alkyl, hydroxyC₁-C₆alkyl, and NH₂C(X)NHC₁-C₆alkyl, wherein X is O or NH, and wherein the aryl part of the arylC₁-C₆alkyl and the heteroaryl part of the heteroarylC₁-C₆alkyl are optionally substituted with one, two, three, four, or five groups independently selected from C₁-C₆alkoxy, C₁-C₆alkyl, amido, amidoC₁-C₆alkyl, amino, aminoC₁-C₆alkyl, carboxy, carboxyC₁-C₆alkoxy, cyano, halo haloC₁-C₆alkyl, hydroxy, and nitro;
R² is selected from aminoC₁-C₆alkyl, arylC₁-C₆alkyl, and heteroarylC₁-C₆alkyl, wherein the aryl part of the arylC₁-C₆alkyl and the heteroaryl part of the heteroarylC₁-C₆alkyl are optionally substituted with one, two, three, four, or five groups independently selected from C₁-C₆alkoxy, C₁-C₆alkyl, amido, amidoC₁-C₆alkyl, amino, aminoC₁-C₆alkyl, carboxy, carboxyC₁-C₆alkoxy, cyano, halo, haloC₁-C₆alkyl, hydroxy, and nitro;
R³ is selected from carboxyC₁-C₃alkyl, cyanoC₁-C₃alkyl and tetrazolylC₁-C₃alkyl;
R⁴ is selected from arylC₁-C₆alkyl and heteroarylC₁-C₆alkyl, wherein the aryl part of the arylC₁-C₆alkyl and the heteroaryl part of the heteroarylC₁-C₆alkyl are optionally substituted with one, two, three, four, or five groups independently selected from C₁-C₆alkoxy, C₁-C₆alkyl,aminoC₁-C₆alkyl, cyano, halo, haloC₁-C₆alkyl, hydroxy, and nitro;
R⁵ is selected from C₁-C₆alkyl, aryl, arylC₁-C₆alkyl, (C₃-C₈cycloalkyl)C₁-C₆alkyl, and hydroxyC₁-C₆alkyl, wherein the aryl part of the arylC₁-C₆alkyl and the heteroaryl part of the heteroarylC₁-C₆alkyl are optionally substituted with one, two, three, four, or five groups independently selected from C₁-C₆alkoxy, C₁-C₆alkyl, amido, amidoC₁-C₆alkyl, amino, aminoC₁-C₆alkyl, carboxy, carboxyC₁-C₆alkoxy, cyano, halo haloC₁-C₆alkyl, hydroxy, and nitro;
R⁶ is selected from aryl-arylC₁-C₃alkyl, heteroaryl-arylC₁-C₃alkyl, aryl-heteroarylC₁-C₃alkyl, and heteroaryl-heteroarylC₁-C₃alkyl wherein the aryl part of the aryl-arylC₁-C₃alkyl and the aryl-heteroarylC₁-C₃alkyl and the heteroaryl part of the heteroaryl-heteroarylC₁-C₃alkyl and the heteroaryl-arylC₁-C₃alkyl is optionally substituted with one, two, three, four, or five groups independently selected from C₁-C₃alkoxy, C₁-C₃alkyl, amido, amidoC₁-C₃alkyl, amino, aminoC₁-C₃alkyl, carboxy, carboxyC₁-C₃alkoxy, cyano, halo, haloC₁-C₃alkyl, hydroxy, and nitro;
R⁷ is selected from C₁-C₆alkyl, aminoC₁-C₆alkyl, arylC₁-C₆alkyl, carboxyC₁-C₆alkyl, and NH₂C(X)NHC₁-C₆alkyl, wherein X is O or NH, wherein the aryl part of the arylC₁-C₆alkyl is optionally substituted with one, two, three, four, or five groups independently selected from C₁-C₆alkoxy, C₁-C₆alkyl, amido, amidoC₁-C₆alkyl, amino, aminoC₁-C₆alkyl, carboxy, carboxyC₁-C₆alkoxy, cyano, halo, haloC₁-C₆alkyl, hydroxy, hydroxyC₁-C₆alkyl, and nitro;
R⁸ is selected from C₁-C₆alkyl, aminoC₁-C₆alkyl, carboxyC₁-C₆alkyl, and NH₂C(X)NHC₁-C₆alkyl, wherein X is O or NH; or, R^{b} and R⁸, together with the atoms to which they are attached, form an azetidine, pyrrolidine, piperidine, or morpholine ring, wherein each ring is optionally substituted with an amino or a hydroxy group;
R⁹ is selected from hydrogen, C₁-C₆alkyl, amidoC₁-C₆alkyl, arylC₁-C₆alkyl, hydroxyC₁-C₆alkyl, C₁-C₆alkoxyC₁-C₆alkyl, and NH₂C(X)NHC₁-C₆alkyl, wherein X is O or NH, wherein the aryl part of the arylC₁-C₆alkyl is optionally substituted with one, two, three, four, or five groups independently selected from C₁-C₆alkoxy, C₁-C₆alkyl, amido, amidoC₁-C₆alkyl, amino, aminoC₁-C₆alkyl, carboxy, carboxyC₁-C₆alkoxy, cyano, halo, haloC₁-C₆alkyl, hydroxy, and nitro; or, R^{c} and R⁹, together with the atoms to which they are attached, form an azetidine, pyrrolidine, piperidine, or morpholine ring, wherein each ring is optionally substituted with an amino or a hydroxy group, and wherein each ring is optionally fused to an aryl or heteroaryl ring, wherein the aryl and heteroaryl rings are optionally substituted with one, two, three, or four groups independently selected from C₁-C₆alkoxy, C₁-C₆alkyl, amido, amidoC₁-C₆alkyl, amino, aminoC₁-C₆alkyl, carboxy, carboxyC₁-C₆alkoxy, cyano, halo haloC₁-C₆alkyl, hydroxy, and nitro;
R¹⁰ is selected from C₁-C₆alkyl, amidoC₁-C₆alkyl, aminoC₁-C₆alkyl, carboxyC₁-C₆alkyl, hydroxyC₁-C₆alkyl, and NH₂C(X)NHC₁-C₆alkyl, wherein X is O or NH;
R¹¹ is selected from C₁-C₈alkyl and (C₃-C₈cycloalkyl)C₁-C₆alkyl, wherein the C₁-C₈alkyl and the (C₃-C₈cycloalkyl)C₁-C₆alkyl are optionally substituted with one, two, or three groups independently selected from C₁-C₆alkoxy, cyano, halo, and haloC₁-C₃alkyl;
R¹² is selected from C₁-C₆alkyl, aminoC₁-C₆alkyl, carboxyC₁-C₆alkyl, hydroxyC₁-C₆alkyl, and NH₂C(X)NHC₁-C₆alkyl, wherein X is O or NH;
R¹³ is selected from amidoC₁-C₆alkyl, aminoC₁-C₆alkyl, carboxyC₁-C₆alkyl, heteroarylC₁-C₆alkyl, hydroxyC₁-C₆alkyl, C₁-C₆alkoxyC₁-C₆alkyl, and NH₂C(X)NHC₁-C₆alkyl, wherein X is O or NH, wherein the heteroaryl part of the heteroarylC₁-C₆alkyl is optionally substituted with one, two, three, four, or five groups independently selected from C₁-C₆alkoxy, C₁-C₆alkyl, amido, amidoC₁-C₆alkyl, amino, aminoC₁-C₆alkyl, carboxy, carboxyC₁-C₆alkoxy, cyano, halo, haloC₁-C₆alkyl, hydroxy, and nitro;
R¹⁴ is —C(O)NH₂ or —C(O)NHCR¹⁵R¹⁶C(O)NH₂, wherein:
R¹¹ is selected from hydrogen and C₁-C₆alkyl; and
R¹⁶ is selected from hydrogen, C₁-C₆alkyl, aminoC₁-C₆alkyl, carboxyC₁-C₆alkyl, (C₃-C₈cycloalkyl)C₁-C₆alkyl, hydroxyC₁-C₆alkyl, and NH₂C(X)NHC₁-C₆alkyl, wherein X is O or NH; or,
R¹⁵ and R¹⁶, together with the carbon atom to which they are attached, form a C₃-C₆cycloalkyl;
R^{a} is hydrogen or C₁-C₆alkyl;
R^{b} is hydrogen, C₁-C₆alkyl, or, R^{b} and R⁸, together with the atoms to which they are attached, form an azetidine, pyrrolidine, piperidine, or morpholine ring, wherein each ring is optionally substituted with an amino or a hydroxy group; and
R^{c} is C₁-C₆alkyl, or R^{c} and R⁹, together with the atoms to which they are attached, form an azetidine, pyrrolidine, piperidine, or morpholine ring, wherein each ring is optionally substituted with an amino or a hydroxy group, and wherein each ring is optionally fused with aryl or heteroaryl ring, wherein the aryl and heteroaryl are optionally substituted with one, two, three, or four groups independently selected from C₁-C₆alkoxy, C₁-C₆alkyl, amido, amidoC₁-C₆alkyl, amino, aminoC₁-C₆alkyl, carboxy, carboxyC₁-C₆alkoxy, cyano, halo haloC₁-C₆alkyl, hydroxy, and nitro.

2. A compound of claim 1, or a pharmaceutically acceptable salt thereof, wherein R² is selected from arylC₁alkyl and heteroarylC₁alkyl, wherein the aryl part of the arylC₁alkyl and the heteroaryl part of the heteroarylC₁alkyl are optionally substituted with one, two, or three groups independently selected from C₁alkoxy, C₁alkyl, amido, amidoC₁alkyl, amino, aminoC₁alkyl, carboxy, carboxyC₁alkoxy, cyano, halo, hydroxy, and nitro.

3. A compound of claim 1 or 2, or a pharmaceutically acceptable salt thereof, wherein R⁴ is each selected from arylC₁alkyl and heteroarylC₁alkyl, wherein the aryl part of the arylC₁alkyl and the heteroaryl part of the heteroarylC₁alkyl are optionally substituted with one, two, or three groups independently selected from C₁alkoxy, C₁alkyl, cyano, halo, haloC₁alkyl, and nitro.

4. A compound of any one of claims 1 to 3, or a pharmaceutically acceptable salt thereof, wherein R³ is carboxyC₁-C₆alkyl.

5. A compound of any one of claims 1 to 4, or a pharmaceutically acceptable salt thereof, wherein R⁵ is selected from C₁-C₆alkyl and arylC₁alkyl, wherein the aryl part of the arylC₁alkyl is optionally substituted with one or two groups independently selected from carboxy and carboxyC₁alkoxy.

6. A compound of any one of claims 1 to 5, or a pharmaceutically acceptable salt thereof, wherein R⁶ is unsubstituted aryl-arylC₁alkyl.

7. A compound of any one of claims 1 to 6, or a pharmaceutically acceptable salt thereof, wherein R^{c} is methyl, or, R^{c} and R⁹, together with the atoms to which they are attached, form an azetidine, morphiline, piperidine, or pyrrolidine ring wherein each ring is optionally substituted with a hydroxy group.

8. A compound of any one of claims 1 to 7, or a pharmaceutically acceptable salt thereof, wherein R¹¹ is selected from C₁-C₆alkyl and (C₃-C₈cycloalkyl)C₁alkyl.

9. A compound of any one of claims 1 to 8, or a pharmaceutically acceptable salt thereof, wherein R² is selected from C₁-C₆alkyl and hydroxyC₁-C₆alkyl.

10. A compound of any one of claims 1 to 9, or a pharmaceutically acceptable salt thereof, wherein R¹³ is selected from hydroxyC₁-C₆alkyl and aminoC₁-C₆alkyl.

11. A compound of claim 1, or a pharmaceutically acceptable salt thereof, wherein:
R¹ is selected from amidoC₁-C₆alkyl, aminoC₁-C₆alkyl, arylC₁-C₆alkyl, and heteroarylC₁-C₆alkyl, wherein the aryl part of the arylC₁-C₆alkyl and the heteroaryl part of the heteroarylC₁-C₆alkyl are optionally substituted with one, two, three, four, or five groups independently selected from C₁-C₆alkoxy, C₁-C₆alkyl, amido, amidoC₁-C₆alkyl, amino, aminoC₁-C₆alkyl, carboxy, carboxyC₁-C₆alkoxy, cyano, halo haloC₁-C₆alkyl, hydroxy, and nitro;
R² is selected from arylC₁-C₆alkyl and heteroarylC₁-C₆alkyl, wherein the aryl part of the arylC₁-C₆alkyl and the heteroaryl part of the heteroarylC₁-C₆alkyl are optionally substituted with one, two, three, four, or five groups independently selected from C₁-C₆alkoxy, C₁-C₆alkyl, amido, amidoC₁-C₆alkyl, amino, aminoC₁-C₆alkyl, carboxy, carboxyC₁-C₆alkoxy, cyano, halo haloC₁-C₆alkyl, hydroxy, and nitro;
R³ is carboxyC₁alkyl;
R⁴ is selected from arylC₁alkyl and heteroarylC₁alkyl, wherein the aryl part of the arylC₁-C₃alkyl and the heteroaryl part of the heteroarylC₁-C₃alkyl are optionally substituted with one, two, three, four, or five groups independently selected from C₁-C₆alkoxy, C₁-C₆alkyl, aminoC₁-C₆alkyl, cyano, halo haloC₁-C₆alkyl, hydroxy, and nitro;
R⁵ is selected from C₁-C₆alkyl, and arylC₁-C₆alkyl, wherein the aryl part of the arylC₁-C₆alkyl and the heteroaryl part of the heteroarylC₁-C₆alkyl are optionally substituted with one, two, three, four, or five groups independently selected from C₁-C₆alkoxy, C₁-C₆alkyl, amido, amidoC₁-C₆alkyl, amino, aminoC₁-C₆alkyl, carboxy, carboxyC₁-C₆alkoxy, cyano, halo haloC₁-C₆alkyl, hydroxy, and nitro;
R⁶ is aryl-arylC₁-C₃alkyl, wherein the aryl or the heteroaryl part is optionally substituted with one, two, three, four, or five groups independently selected from C₁-C₆alkoxy, C₁-C₆alkyl, amino, aminoC₁-C₆alkyl, cyano, halo haloC₁-C₆alkyl, hydroxy, and nitro;
R⁷ is selected from C₁-C₆alkyl, carboxyC₁-C₆alkyl, NH₂C(O)NHC₁-C₆alkyl;
R⁸ is arylC₁-C₆alkyl
R¹⁰ is selected from amidoC₁-C₆alkyl and aminoC₁-C₆alkyl;
R¹¹ is selected from C₁-C₆alkyl and (C₃-C₈cycloalkyl)C₁-C₆alkyl;
R¹² is selected from C₁-C₆alkyl and hydroxyC₁-C₆alkyl;
R¹³ is hydroxyC₁-C₆alkyl and aminoC₁-C₆alkyl;
R¹⁴ is —C(O)NHCR¹⁵R¹⁶C(O)NH₂, wherein:
R¹⁵ is hydrogen; and
R¹⁶ is selected from C₁-C₆alkyl and aminoC₁-C₆alkyl;
R^{a} is hydrogen;
R^{b} is hydrogen or methyl;
R^{c} is C₁-C₆alkyl, or R^{c} and R⁹, together with the atoms to which they are attached, form a pyrrolidine ring of formula: 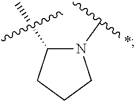
wherein “ [symbol] ” is the point of attachment to the —C(O)NH group, and “ [symbol] ” is the point of attachment to the —C(O)CH^{R} group.

12. A compound of claim 11, or a pharmaceutically acceptable salt thereof, wherein R¹ is selected from amidoC₁alkyl, aminoC₁-2alkyl, arylC₁alkyl, and heteroarylC₁alkyl, wherein the aryl part of the arylC₁alkyl is optionally substituted with a carboxyC₁alkoxy group.

13. A compound of claim 11 or claim 12, or a pharmaceutically acceptable salt thereof, wherein R² is selected from arylC₁alkyl and heteroarylC₁alkyl, wherein the aryl part of the arylC₁alkyl is optionally substituted with one group selected from carboxy, carboxyC₁alkoxy, and cyano.

14. A compound of any one of claims 11 to 13, or a pharmaceutically acceptable salt thereof, wherein the aryl is a phenyl or naphthyl group, and wherein the heteroaryl is a benzothienyl, imidazolyl, indolyl, pyrazolyl, pyridinyl, or thiazolyl group.

15. A compound of any one of claims 11 to 14, or a pharmaceutically acceptable salt thereof, wherein R³ is carboxyC₁alkyl.

16. A compound of any one of claims 11 to 15, or a pharmaceutically acceptable salt thereof, wherein R⁴ is selected from heteroarylC₁alkyl, wherein the heteroaryl is indolyl, and arylC₁alkyl, wherein the aryl part of the arylC₁alkyl is optionally substituted with one group selected from C₁alkoxy and C₁alkyl.

17. A compound of any one of claims 11 to 16, or a pharmaceutically acceptable salt thereof, wherein R⁵ is selected from C₃-C₄alkyl, and arylC₁alkyl, wherein the aryl part of the arylC₁alkyl is optionally substituted with one carboxyC₁alkoxy group.

18. A compound of any one of claims 11 to 17, or a pharmaceutically acceptable salt thereof, wherein R⁶ is unsubstituted aryl-arylC₁alkyl.

19. A compound of any one of claims 11 to 18, or a pharmaceutically acceptable salt thereof, wherein R⁷ is selected from C₃alkyl, carboxyC₂alkyl, and NH₂C(O)NHC₁alkyl.

20. A compound of any one of claims 11 to 19, or a pharmaceutically acceptable salt thereof, wherein R⁸ is selected from C₁alkyl and R^{b} is methyl, and Re is selected from aminoC₃alkyl and R^{b} is hydrogen.

21. A compound of any one of claims 11 to 20, or a pharmaceutically acceptable salt thereof, wherein R⁹ is arylC₁alkyl and R^{c} is methyl, or R^{c} and R⁹, together with the atoms to which they are attached, form a pyrrolidine ring of formula: 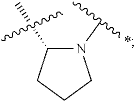
wherein “ [symbol] ” is the point of attachment to the —C(O)NH group, and “ [symbol] ” is the point of attachment to the —C(O)CHR⁸ group.

22. A compound of any one of claims 11 to 21, or a pharmaceutically acceptable salt thereof, wherein R¹⁰ is selected from amidoC₁alkyl and aminoC₂alkyl.

23. A compound of any one of claims 11 to 22, or a pharmaceutically acceptable salt thereof, wherein R¹¹ is selected from C₄alkyl and (C₆cycloalkyl)C₁alkyl.

24. A compound of any one of claims 11 to 23, or a pharmaceutically acceptable salt thereof, wherein R¹² is selected from C₃alkyl and hydroxyC₃alkyl.

25. A compound of any one of claims 11 to 24, or a pharmaceutically acceptable salt thereof, wherein R³ is hydroxyC₁-C₂alkyl.

26. A compound of any one of claims 11 to 25, or a pharmaceutically acceptable salt thereof, wherein R¹⁶ is selected from C₁alkyl and aminoC₂alkyl.

27. A pharmaceutical composition comprising a compound of any one of claims 1 to 26, or a pharmaceutically acceptable salt thereof.

28. A method of enhancing, stimulating, and/or increasing an immune response in a subject in need thereof, wherein the method comprises administering to the subject a therapeutically effective amount of a compound of any one of claims 1 to 26, or a pharmaceutically acceptable salt thereof.

29. A method of blocking the interaction of PD-1 with PD-L1 in a subject, wherein the method comprises administering to the subject a therapeutically effective amount of a compound of any one of claims 1 to 26 or a pharmaceutically acceptable salt thereof.


SEQUENCE LISTING (1 sequence)

SEQ ID NO: 1 - protein, 470 aa, synthetic construct
  REGION 1..470: note = hPDL1(18-239)-TVMV-mIgG1(221-447)-C225S
AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTGSPGGGGGRETVRFQGGTGDAVPRDSGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK